(19) United States
(12) Patent Application Publication
(10) Pub. No.: US 2025/0186437 A1
(43) Pub. Date: Jun. 12, 2025

(54) CARBOLINE COMPOUNDS AND USE THEREOF

(71) Applicant: PTC Therapeutics, Inc., South Plainfield, NJ (US)

(72) Inventors: Ramil Baiazitov, East Brunswick, NJ (US); Liangxian Cao, East Brunswick, NJ (US); Matteo Chierchia, Jersey City, NJ (US); Bradley B. Gilbert, Milford, NJ (US); Woohyung Jeon, Fort Lee, NJ (US); Malte Mikus, Biberach (DE); Young-Choon Moon, Warren, NJ (US); Zachary Powers, Bedminster, NJ (US); Hongyu Ren, Florence, NJ (US); Gang Wang, Edison, NJ (US); Jin Zhuo, Hillsborough, NJ (US)

(21) Appl. No.: 18/725,509

(22) PCT Filed: Dec. 30, 2022
(86) PCT No.: PCT/US2022/082622

§ 371 Date: Jun. 28, 2024

Related U.S. Application Data
(60) Provisional application No. 63/295,844, filed on Dec. 31, 2021.

(51) Int. Cl. A61K 31/506; A61K 31/5377; C07D 471/04

(52) U.S. Cl. CPC A61K 31/506; A61K 31/5377; C07D 471/04

(57) ABSTRACT

Provided herein are β-carboline compounds and their use to inhibit dihydroorotate dehydrogenase (DHODH):

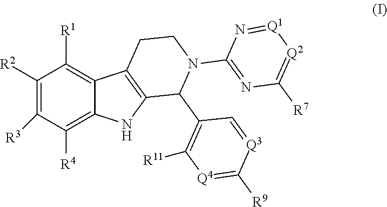

or a form thereof,
wherein R¹, R², R³, R⁴, Q¹, Q², Q³, Q⁴, R⁷, R⁹, and R¹¹ are as defined herein.

### CROSS-REFERENCE TO RELATED APPLICATIONS

This application is a national phase entry under 35 USC § 371 of International Application PCT/US2022/082622, filed Dec. 30, 2022, which claims the benefit of and priority to U.S. Provisional Application No. 63/295,844 filed Dec. 31, 2021, the entireties of which are herein incorporated by reference.

### FIELD

Provided herein are R-carboline compounds and their use to inhibit dihydroorotate dehydrogenase (DHODH).

### BACKGROUND

Dihydroorotate dehydrogenase (DHODH) is located on the inner membrane of mitochondria and acts in the de novo pyrimidine nucleotide synthesis pathway to catalyze dehydrogenation of dihydroorotate (DHO) to orotic acid (ORO), resulting in the generation of uridine monophosphate (UMP) (Munier-Lehmann et al., J Med Chem 2015; 58(2):860-877). UMP is subsequently converted to uridine (U) and cytosine (C) triphosphates to supply the cellular pool of pyrimidine nucleotides.

Pyrimidine nucleotides are supplied via both de novo biosynthesis and salvage pathways, as illustrated in FIG. 1. The salvage pathway recycles nucleotides from nucleosides and free bases generated by DNA and RNA breakdown. For highly proliferative activated T cells, malignant cancer cells, and rapid replicative RNA virus, de novo pyrimidine nucleotide synthesis is essential as salvage pathways have insufficient capacity to satisfy the increased demand for biosynthesis of nucleic acids and membrane phospholipids.

DHODH is a rate-limiting enzyme for the de novo synthesis of pyrimidine ribonucleotides. As such, inhibitors of DHODH have been used to treat autoimmune diseases and are in clinical trials for cancer and viral infections. In general, inhibitors of DHODH show beneficial immunosuppressive and antiproliferative activities, with pronounced effects on activated lymphocyte proliferation. Many existing DHODH inhibitors have been reported including for example, leflunomide, teriflunomide, brequinar, maritimus (FK 778), redoxal, BAY2402234, ASLAN003, and emvodostat (PTC299). Leflunomide is used in the treatment of rheumatoid arthritis, but it is known to have off-target effects and a very long half-life. Emvodostat was in clinical trials (Clinical Trials.gov Identifier: NCT03761069) for the treatment of acute myeloid leukemia, while BAY2402234 was tested for use in the treatment of acute myeloid leukemia and recurrent glioma. Emvodostat has also been shown to inhibit RNA virus infections and was in clinical trials (Clinical Trials.gov Identifier: NCT04439071) for the treatment of COVID-19. Studies also indicate that emvodostat can inhibit the cytokine storm associated with COVID-19 infections (see, Luban et al, Virus Research 292 (2021), 190246).

Emvodostat has the following structure:

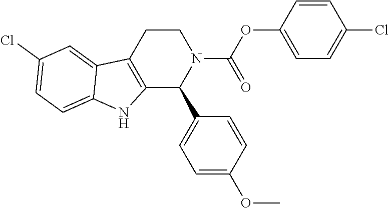 [SMILES: COc1ccc([C@H]2c3[nH]c4ccc(Cl)cc4c3CCN2C(=O)Oc2ccc(Cl)cc2)cc1]

The following published patents and patent applications describe compositions, methods of making and/or using emvodostat: WO2005089764, U.S. Pat. No. 7,601,840, WO2010138644, U.S. Ser. No. 11/458,126, WO2010138758, U.S. Pat. No. 9,351,964, WO2019028171, U.S. Ser. No. 11/458,126, WO2020028778, US20210205225, WO2021226478, and U.S. Ser. No. 10/947,231.

Although several DHODH inhibitors currently in development show promise, there is an ongoing need for new, potent, therapeutically beneficial compounds useful as DHODH inhibitors, and new compositions thereof.

### SUMMARY

Provided herein is a compound of Formula (I):

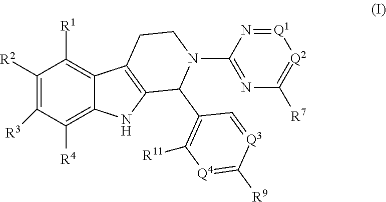

or a form thereof, wherein:
R¹ is hydrogen, deuterium, amino, nitro or fluoro;
R² is halo, C₁₋₄alkyl, C₂₋₄alkenyl, nitro, C₁₋₄alkoxy, (C₁₋₄alkyl)₂amino, pyrrolyl, pyrazolyl, 3-methyl-pyrazolyl, 4-methyl-pyrazolyl, 2H-1,2,3-triazol-2-yl, or 4-methyl-2H-1,2,3-triazolyl-2-yl;
R³ is hydrogen, deuterium, amino or fluoro;
R⁴ is hydrogen, amino or hydroxy;
Q¹ is CR³ or N;
Q² is CR⁶ or N;
wherein Q¹ and Q² are not simultaneously N;
R⁵ and R⁷ are independently selected from hydrogen, deuterium, halo, cyano, C₁₋₄alkyl, halo-C₁₋₄alkyl, hydroxy-C₁₋₄alkyl, C₁₋₄alkyl-thio, C₁₋₄alkoxy, amino, (C₁₋₄alkyl)amino, (C₁₋₄alkyl)₂amino, C₁₋₄alkoxycarbonyl, phenyl and heterocyclyl, wherein heterocyclyl is a saturated or partially unsaturated 3-7 membered monocyclic ring system radical having one, two, or three heteroatom ring members independently selected from N, O, or S; and, wherein phenyl and heterocyclyl are optionally substituted with one, two, or three R¹² substituents;
R⁶ is hydrogen, deuterium, halo, C₁₋₆alkyl, halo-C₁₋₄alkyl, phenyl or morpholinyl;
wherein, when Q² is N, then R⁵ and R⁶ are other than halo; and,
wherein R³, R⁶ and R⁷ are not all simultaneously hydrogen;
Q³ is CR⁸ or N;
R⁸ is hydrogen or deuterium;
R⁹ is hydrogen, deuterium, halo, cyano, hydroxy, C₁₋₄alkyl, halo-C₁₋₄alkyl, C₂₋₄alkenyl, C₁₋₄alkylcarbonyl, amino, (C₁₋₄alkyl)amino, (C₁₋₄alkyl)₂amino, (C₁₋₄alkyl)₂amino(C₁₋₄alkyl)amino, C₁₋₄alkylthio, C₁₋₄alkyl-sulfonyl, amino-sulfonyl, C₁₋₄alkoxy, C₁₋₄alkoxy-C₁₋₄alkoxy, (C₁₋₄alkoxy)carbonyl, (C₁₋₄alkoxy)carbonylC₁₋₄alkyl, hydroxyC₁₋₄alkyl, hydroxyC₂₋₄alkenyl, hydroxyC₁₋₄alkylamino, hydroxyC₁₋₄alkoxyC₁₋₄alkyl, carboxyl_C₁₋₄alkyl, carboxyl_C₁₋₄alkylamino, carboxyl_C₁₋₄alkoxy-C₁₋₄alkylamino, C₃₋₆cycloalkyl, heteroaryl or heterocyclyl, wherein heteroaryl is an aromatic 5-6 membered monocyclic ring having one, two, or three heteroatom ring members independently selected from N, O, or S; wherein heterocyclyl is a saturated or partially unsaturated 3-7 membered monocyclic, 6-10 membered bicyclic, 7-8 membered bicyclic, or 13-16 membered polycyclic ring system having one, two, or three heteroatom ring members independently selected from N, O, or S; and, wherein each heteroaryl or heterocyclyl is optionally substituted with one, two, or three R¹² substituents;
Q⁴ is CR¹⁰ or N;
R¹⁰ is hydrogen, deuterium, fluoro or hydroxy;
R¹¹ is hydrogen, deuterium, fluoro or hydroxy; and,
wherein R⁸, R⁹, R¹⁰ and R¹¹ are not simultaneously hydrogen; and,
R¹² is halo, hydroxy, C₁₋₆alkyl or C₃₋₆cycloalkyl; and,
wherein the form of the compound is selected from the group consisting of a salt, hydrate, solvate, racemate, enantiomer, diastereomer, stereoisomer, and tautomer thereof.

Another aspect, provided herein is a pharmaceutical composition comprising a compound of Formula (I) or a form thereof, and a pharmaceutically acceptable excipient.

Another aspect, provided herein is a method of treating a disease or disorder amenable to dihydroorotate dehydrogenase inhibition using a compound of Formula (I), or a form thereof, or pharmaceutical composition thereof.

### BRIEF DESCRIPTION OF THE DRAWINGS

FIG. 1 is a diagram illustrating the salvage and de novo pathways for pyrimidine synthesis. The terms “dihydroorotate dehydrogenase inhibition” or “DHODH inhibition” refer to inhibition of pyrimidine synthesis via the de novo pathway in the presence of a DHODH inhibitor such as those compounds disclosed herein.

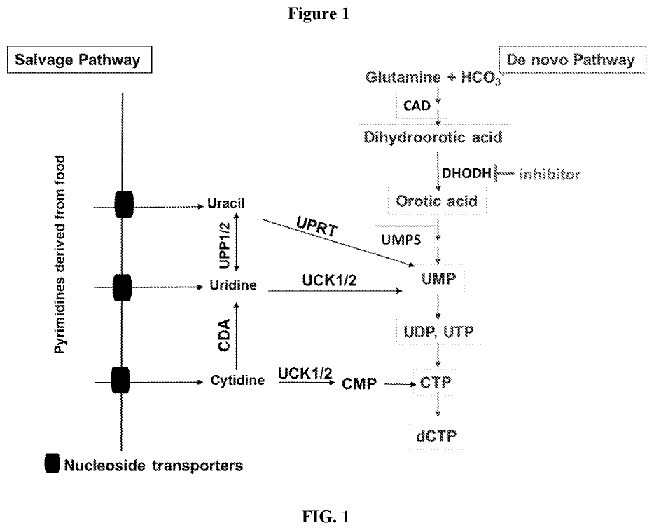

### DETAILED DESCRIPTION

In one aspect provided herein is a compound of Formula (I):

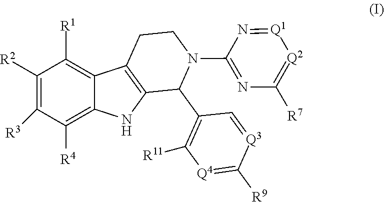

or a form thereof, wherein:
R¹ is hydrogen, deuterium, amino, nitro or fluoro;
R² is halo, C₁₋₄alkyl, C₂₋₄alkenyl, nitro, C₁₋₄alkoxy, (C₁₋₄alkyl)₂amino, pyrrolyl, pyrazolyl, 3-methyl-pyrazolyl, 4-methyl-pyrazolyl, 2H-1,2,3-triazol-2-yl, or 4-methyl-2H-1,2,3-triazolyl-2-yl;
R³ is hydrogen, deuterium, amino or fluoro;
R⁴ is hydrogen, amino or hydroxy;
Q¹ is CR³ or N;
Q² is CR⁶ or N;
wherein Q¹ and Q² are not simultaneously N;
R⁵ and R⁷ are independently selected from hydrogen, deuterium, halo, cyano, C₁₋₄alkyl, halo-C₁₋₄alkyl, hydroxy-C₁₋₄alkyl, C₁₋₄alkyl-thio, C₁₋₄alkoxy, amino, (C₁₋₄alkyl)amino, (C₁₋₄alkyl)₂amino, C₁₋₄alkoxycarbonyl, phenyl and heterocyclyl, wherein heterocyclyl is a saturated or partially unsaturated 3-7 membered monocyclic ring system radical having one, two, or three heteroatom ring members independently selected from N, O, or S; and, wherein phenyl and heterocyclyl are optionally substituted with one, two, or three R¹² substituents;
R⁶ is hydrogen, deuterium, halo, C₁₋₆alkyl, halo-C₁₋₄alkyl, phenyl or morpholinyl;
wherein, when Q² is N, then R⁵ and R⁶ are other than halo; and
wherein R³, R⁶ and R⁷ are not all simultaneously hydrogen;
Q³ is CR⁸ or N;
R⁸ is hydrogen or deuterium;
R⁹ is hydrogen, deuterium, halo, cyano, hydroxy, C₁₋₄alkyl, halo-C₁₋₄alkyl, C₂₋₄alkenyl, C₁₋₄alkylcarbonyl, amino, (C₁₋₄alkyl)amino, (C₁₋₄alkyl)₂amino, (C₁₋₄alkyl)₂amino(C₁₋₄alkyl)amino, C₁₋₄alkylthio, C₁₋₄alkyl-sulfonyl, amino-sulfonyl, C₁₋₄alkoxy, C₁₋₄alkoxyC₁₋₄alkoxy, (C₁₋₄alkoxy)carbonyl, (C₁₋₄alkoxy)carbonylC₁₋₄alkyl, hydroxyC₁₋₄alkyl, hydroxyC₂₋₄alkenyl, hydroxyC₁₋₄alkylamino, hydroxyC₁₋₄alkoxyC₁₋₄alkyl, carboxyl-C₁₋₄alkyl, carboxyl_C₁₋₄alkyl_amino, carboxyl_C₁₋₄alkoxy-C₁₋₄alkylamino, C₃₋₆cycloalkyl, heteroaryl or heterocyclyl, wherein heteroaryl is an aromatic 5-6 membered monocyclic ring having one, two, or three heteroatom ring members independently selected from N, O, or S, wherein heterocyclyl is a saturated or partially unsaturated 3-7 membered monocyclic, 6-10 membered bicyclic, 7-8 membered bicyclic, or 13-16 membered polycyclic ring system having one, two, or three heteroatom ring members independently selected from N, O, or S, and, wherein each heteroaryl or heterocyclyl is optionally substituted with one, two, or three R¹² substituents;
Q⁴ is CR¹⁰ or N;
R¹⁰ is hydrogen, deuterium, fluoro or hydroxy;
R¹¹ is hydrogen, deuterium, fluoro or hydroxy; and
wherein R⁸, R⁹, R¹⁰ and R¹¹ are not simultaneously hydrogen; and,
R¹² is halo, hydroxy, C₁₋₆alkyl or C₃₋₆cycloalkyl; and,
wherein the form of the compound is selected from the group consisting of a salt, hydrate, solvate, racemate, enantiomer, diastereomer, stereoisomer, and tautomer thereof.

Another aspect includes a compound of Formula (I) or form thereof wherein the form is a salt thereof.

Another aspect includes a compound of Formula (I), wherein at least two of R¹, R³ and R⁴ are hydrogen.

Another aspect includes a compound of Formula (I), wherein R¹, R³ and R⁴ are each hydrogen.

Another aspect includes a compound of Formula (I), wherein R¹ is hydrogen, amino, nitro or fluoro;

Another aspect includes a compound of Formula (I), wherein R² is halo, C₁₋₄alkyl, C₂₋₄alkenyl, nitro, C₁₋₄alkoxy or (C₁₋₄alkyl)₂amino.

Another aspect includes a compound of Formula (I), wherein R² is pyrrolyl, pyrazolyl, 3-methyl-pyrazolyl, 4-methyl-pyrazolyl, 2H-1,2,3-triazol-2-yl, or 4-methyl-2H-1,2,3-triazolyl-2-yl.

Another aspect includes a compound of Formula (I), wherein R² is chloro, fluoro, bromo, methyl, ethyl, ethenyl, nitro, methoxy, dimethyl-amino, 1H-pyrrol-1-yl, 1H-pyrazol-1-yl, 3-methyl-1H-pyrazol-1-yl, 4-methyl-1H-pyrazol-1-yl, 2H-1,2,3-triazol-2-yl, or 4-methyl-2H-1,2,3-triazol-2-yl.

Another aspect includes a compound of Formula (I), wherein R² is halo or 2H-1,2,3-triazol-2-yl.

Another aspect includes a compound of Formula (I), wherein R² is halo.

Another aspect includes a compound of Formula (I), wherein R² is halo, wherein halo is selected from chloro or bromo.

Another aspect includes a compound of Formula (I), wherein R² is chloro, fluoro, bromo, methyl, ethenyl, nitro, methoxy, dimethyl-amino, 1H-pyrrol-1-yl, 1H-pyrazol-1-yl, 2H-1,2,3-triazol-2-yl, or 4-methyl-2H-1,2,3-triazol-2-yl.

Another aspect includes a compound of Formula (I), wherein R⁵ and R⁷ are independently selected from hydrogen, deuterium, halo, cyano, C₁₋₄alkyl, halo-C₁₋₄alkyl, hydroxy-C₁₋₄alkyl, C₁₋₄alkyl-thio, C₁₋₄alkoxy, amino, (C₁₋₄alkyl)amino, (C₁₋₄alkyl)₂amino, C₁₋₄alkoxycarbonyl, phenyl or heterocyclyl, wherein heterocyclyl is selected from morpholinyl, piperazinyl and azetidinyl, and wherein heterocyclyl is optionally substituted with one, two, or three R¹² substituents.

Another aspect includes a compound of Formula (I), wherein R⁵ and R⁷ are independently selected from hydrogen, halo, cyano, C₁₋₄alkyl, halo-C₁₋₄alkyl, hydroxy-C₁₋₄alkyl, C₁₋₄alkyl-thio, C₁₋₄alkoxy, amino, (C₁₋₄alkyl)₂amino, C₁₋₄alkoxycarbonyl, phenyl, or heterocyclyl, wherein heterocyclyl is selected from morpholinyl, piperazinyl and azetidinyl, and wherein heterocyclyl is optionally substituted with one, two, or three R¹² substituents.

Another aspect includes a compound of Formula (I), wherein R⁵ and R⁷ are independently selected from hydrogen, fluoro, chloro, bromo, cyano, methyl, ethyl, isopropyl, fluoromethyl, difluoromethyl, trifluoromethyl, trichloromethyl, hydroxy-methyl, methyl-thio, methoxy, butoxy, amino, N,N-dimethyl-amino, N-methyl-N-butyl-amino, N,N-dibutyl-amino, methoxy-carbonyl, butoxy-carbonyl, phenyl, morpholinyl, 3,3-difluoro-azetidin-1-yl, 1-methylpiperazin-4-yl, or phenyl.

Another aspect includes a compound of Formula (I), wherein at least one of R⁵ and R⁷ is methyl or trifluoromethyl.

Another aspect includes a compound of Formula (I), wherein at least one of R⁵ and R⁷ is trifluoromethyl.

Another aspect includes a compound of Formula (I), wherein R⁶ is selected from hydrogen, halo, methyl, trifluoromethyl, or phenyl.

Another aspect includes a compound of Formula (I), where R⁶ is selected from hydrogen, chloro, fluoro, bromo, methyl, trifluoromethyl, or phenyl.

Another aspect includes a compound of Formula (I), wherein R⁸ is hydrogen.

Another aspect includes a compound of Formula (I), wherein R⁹ is hydrogen, deuterium, halo, cyano, C₁₋₄alkyl, halo-C₁₋₄alkyl, C₂₋₄alkenyl, C₁₋₄alkylcarbonyl, amino, (C₁₋₄alkyl)amino, (C₁₋₄alkyl)₂amino, (C₁₋₄alkyl)₂amino(C₁₋₄alkyl)amino, C₁₋₄alkylthio, C₁₋₄alkyl-sulfonyl, amino_sulfonyl, C₁₋₄alkoxy, C₁₋₄alkoxy-C₁₋₄alkoxy, (C₁₋₄alkoxy)carbonyl, hydroxyC₁₋₄alkyl, hydroxyC₂₋₄alkenyl, hydroxyC₁₋₄alkylamino, (C₁₋₄alkoxy)carbonylC₁₋₄alkyl, hydroxyC₁₋₄alkoxyC₁₋₄alkyl, carboxyl-C₁₋₄alkyl, carboxyl-C₁₋₄alkylamino, carboxyl-C₁₋₄alkoxy-C₁₋₄alkylamino, C₃₋₆cycloalkyl, heteroaryl or heterocyclyl, wherein heteroaryl is an unsaturated 5-6 membered monocyclic ring having one, two, or three heteroatom ring members independently selected from N, O, or S, wherein heterocyclyl is a saturated or partially unsaturated 7-8 membered bicyclic ring system having one, two, or three heteroatom ring members independently selected from N, O, or S, and, wherein each heterocyclyl or heteroaryl is optionally substituted with one, two, or three R² substituents.

Another aspect includes a compound of Formula (I), wherein R⁹ optionally substituted heteroaryl is

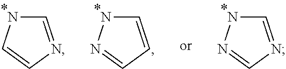

and, wherein optionally substituted heterocyclyl is selected from

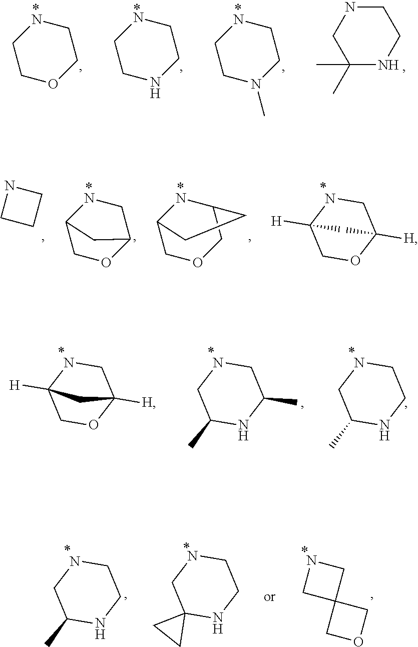

and, wherein the asterisk (*) designates the point of attachment.

Another aspect includes a compound of Formula (I), wherein R⁹ optionally substituted heteroaryl is 1H-imidazolyl, 1H-pyrazolyl or 1H-1,2,4-triazolyl; and, wherein optionally substituted heterocyclyl is selected from morpholinyl, piperazinyl, 4-methyl-piperazinyl, 3,3-dimethyl-piperazinyl, azetidinyl, 2-oxa-5-azabicyclo[2.2.1]heptanyl, 3-oxa-8-azabicyclo[3.2.1]octanyl, (1S,4S)-2-oxa-5-azabicyclo[2.2.1]heptanyl, (1R,4R)-2-oxa-5-azabicyclo[2.2.1]heptanyl, (3R,5S)-3,5-dimethylpiperazinyl, (3R)-3-methylpiperazinyl, (3S)-3-methylpiperazinyl, 4,7-diazaspiro[2.5]octanyl, or 2-oxa-6-azaspiro[3.3]heptanyl.

Another aspect includes a compound of Formula (I), wherein R⁹ optionally substituted heteroaryl is 1H-imidazol-1-yl, 1H-pyrazol-1-yl or 1H-1,2,4-triazol-1-yl; wherein optionally substituted heterocyclyl is selected from morpholin-4-yl, piperazin-1-yl, 4-methyl-piperazin-1-yl, 3,3-dimethyl-piperazin-1-yl, azetidin-1-yl, 2-oxa-5-azabicyclo[2.2.1]heptan-5-yl, 3-oxa-8-azabicyclo[3.2.1]octan-8-yl, (1S,4S)-2-oxa-5-azabicyclo[2.2.1]heptan-5-yl, (1R,4R)-2-oxa-5-azabicyclo[2.2.1]heptan-5-yl, (3R,5S)-3,5-dimethylpiperazin-1-yl, (3R)-3-methylpiperazin-1-yl, (3S)-3-methylpiperazin-1-yl, 4,7-diazaspiro[2.5]octan-7-yl, or 2-oxa-6-azaspiro[3.3]heptan-6-yl.

Another aspect includes a compound of Formula (I) wherein R⁹ is hydrogen, fluoro, chloro, bromo, cyano, methyl, isopropyl, trifluoromethyl, ethenyl, methylcarbonyl, amino, N-methylamino, N,N-dimethylamino, N—[(N,N-dimethylamino)ethyl]amino, methyl-thio, methyl-sulfonyl, amino-sulfonyl, methoxy, methoxyethoxy, methoxycarbonyl, hydroxy-methyl, hydroxyethyl, hydroxypropyl, hydroxybutyl, 1-hydroxy-but-3-en-4-yl, N-(hydroxyethyl)amino, methoxy-carbonyl-ethyl, hydroxy-ethoxy-methyl, carboxy-ethyl, carboxy-propyl, N-(carboxy-ethyl)amino, N-(carboxy-propyl)amino, N-(carboxy-methoxy-ethyl)amino, cyclopropyl,

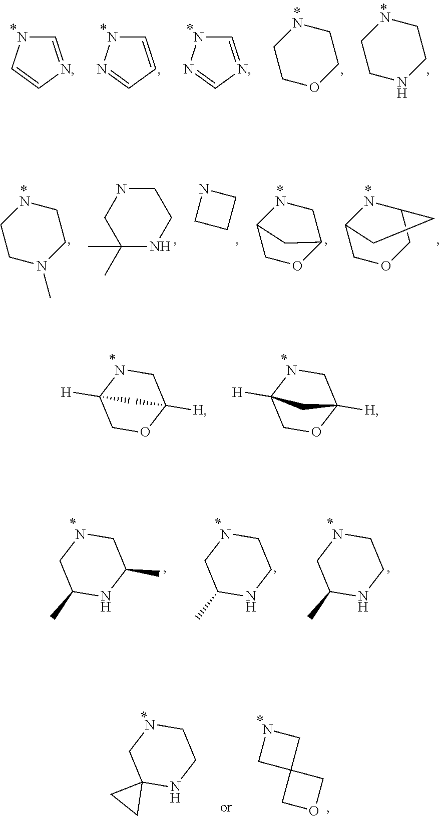

wherein the asterisk (*) designates the point of attachment.

Another aspect includes a compound of Formula (I) wherein R⁹ is hydrogen, fluoro, chloro, bromo, cyano, methyl, isopropyl, trifluoromethyl, ethenyl, methylcarbonyl, amino, 1N-methylamino, N,N-dimethylamino, N—[(N,N-dimethylamino)ethyl]amino, methyl-thio, methyl-sulfonyl, amino-sulfonyl, methoxy, methoxyethoxy, methoxycarbonyl, hydroxymethyl, hydroxyethyl, hydroxypropyl, hydroxybutyl, 1-hydroxy-but-3-en-4-yl, N-(hydroxyethyl)amino, methoxy-carbonyl-ethyl, hydroxy-ethoxy-methyl, carboxy-ethyl, carboxy-propyl, N-(carboxy-ethyl)amino, N-(carboxy-propyl)amino, N-(carboxy-methoxy-ethyl)amino, cyclopropyl, 1H-imidazolyl, 1H-pyrazolyl, 1H-1,2,4-triazolyl, morpholinyl, piperazinyl, 4-methyl-piperazinyl, 3,3-dimethyl-piperazinyl, azetidinyl, 2-oxa-5-azabicyclo[2.2.1]heptanyl, 3-oxa-8-azabicyclo[3.2.1]octanyl, (1S,4S)-2-oxa-5-azabicyclo[2.2.1]heptanyl, (1R,4R)-2-oxa-5-azabicyclo[2.2.1]heptanyl, (3R,5S)-3,5-dimethylpiperazinyl, (3R)-3-methylpiperazinyl, (3S)-3-methylpiperazinyl, 4,7-diazaspiro[2.5]octanyl, or 2-oxa-6-azaspiro[3.3]heptanyl.

Another aspect includes a compound of Formula (I) wherein R⁹ is hydrogen, fluoro, chloro, bromo, cyano, methyl, isopropyl, trifluoromethyl, ethenyl, methylcarbonyl, amino, N-methylamino, N,N-dimethylamino, N—[(N,N-dimethylamino)ethyl]amino, methyl-thio, methyl-sulfonyl, amino-sulfonyl, methoxy, methoxyethoxy, methoxycarbonyl, hydroxymethyl, hydroxyethyl, hydroxypropyl, hydroxybutyl, 1-hydroxy-but-3-en-4-yl, N-(hydroxyethyl)amino, methoxy-carbonyl-ethyl, hydroxy-ethoxy-methyl, carboxy-ethyl, carboxy-propyl, N-(carboxy-ethyl)amino, N-(carboxy-propyl)amino, N-(carboxy-methoxy-ethyl)amino, cyclopropyl, 1H-imidazol-1-yl, 1H-pyrazol-1-yl or 1H-1,2,4-triazol-1-yl, morpholin-4-yl, piperazin-1-yl, 4-methyl-piperazin-1-yl, 3,3-dimethyl-piperazin-1-yl, azetidin-1-yl, 2-oxa-5-azabicyclo[2.2.1]heptan-5-yl, 3-oxa-8-azabicyclo[3.2.1]octan-8-yl, (1S,4S)-2-oxa-5-azabicyclo[2.2.1]heptan-5-yl, (1R,4R)-2-oxa-5-azabicyclo[2.2.1]heptan-5-yl, (3R,5S)-3,5-dimethylpiperazin-1-yl, (3R)-3-methylpiperazin-1-yl, (3S)-3-methylpiperazin-1-yl, 4,7-diazaspiro[2.5]octan-7-yl, or 2-oxa-6-azaspiro[3.3]heptan-6-yl.

Another aspect includes a compound of Formula (I), wherein R⁹ is selected from hydrogen, halo, C₁₋₄alkyl, halo-C₁₋₄alkyl, hydroxy-C₁₋₄alkyl, C₁₋₄alkoxy, morpholinyl or 1-methyl-piperazin-4-yl.

Another aspect includes a compound of Formula (I), wherein R⁹ is selected from hydrogen, halo, methyl, methoxy, trifluoromethyl, or hydroxy-methyl.

Another aspect includes a compound of Formula (I), wherein R¹⁰ is hydrogen, fluoro or hydroxy.

Another aspect includes a compound of Formula (I), wherein R¹¹ is hydrogen, fluoro or hydroxy.

One aspect of the compound of Formula (I) includes a compound of Formula (I) or a form thereof selected from a compound of Formula (Ia1), a compound of Formula (Ia2), a compound of Formula (Ib), a compound of Formula (Ic), a compound of Formula (Id), and a compound of Formula (Ie):

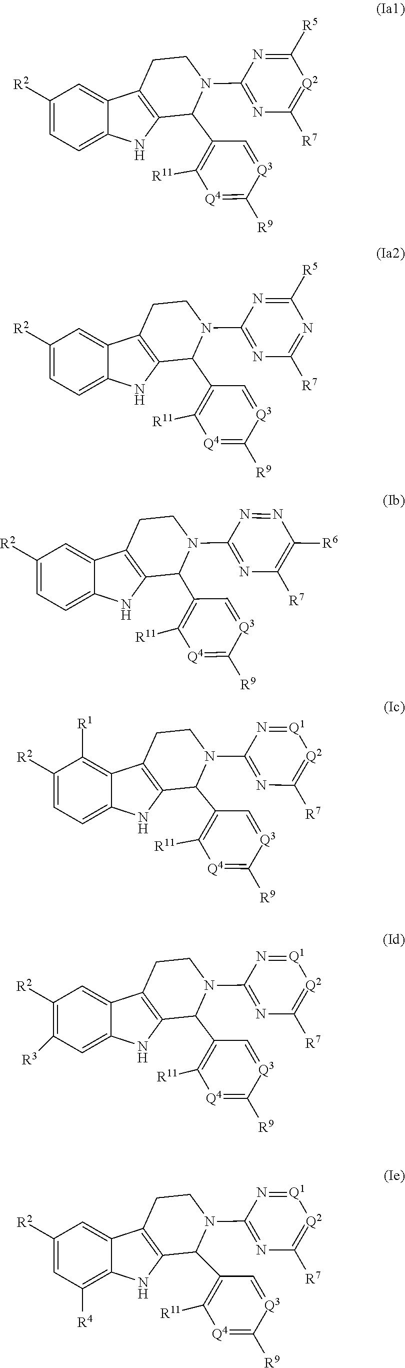

or a form thereof, wherein R¹, R², R³, R⁴, R⁵, R⁶, R⁷, R⁹, R¹¹, Q¹, Q², Q³ and Q⁴ are as defined in the first aspect of Formula (I), above.

Another aspect of the compound of Formula (I) includes a compound of Formula (I) or a form thereof selected from a compound of Formula (Ia1):

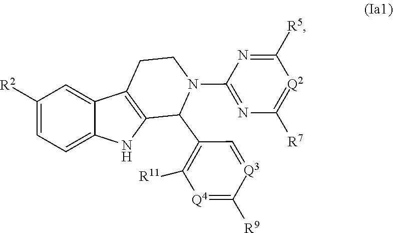

or a form thereof, wherein:
R² is halo, C₁₋₄alkyl, C₂₋₄alkenyl, nitro, C₁₋₄alkoxy, (C₁₋₄alkyl)₂amino, pyrrolyl, pyrazolyl, 3-methyl-pyrazolyl, 4-methyl-pyrazolyl, 2H-1,2,3-triazol-2-yl, or 4-methyl-2H-1,2,3-triazolyl-2-yl;
Q² is CR⁶ or N;
R⁵ and R⁷ are independently selected from hydrogen, deuterium, halo, cyano, C₁₋₄alkyl, halo-C₁₋₄alkyl, hydroxy-C₁₋₄alkyl, C₁₋₄alkyl-thio, C₁₋₄alkoxy, amino, (C₁₋₄alkyl)amino, (C₁₋₄alkyl)₂amino, C₁₋₄alkoxycarbonyl, phenyl and heterocyclyl, wherein heterocyclyl is a saturated or partially unsaturated 3-7 membered monocyclic ring system radical having one, two, or three heteroatom ring members independently selected from N, O, or S; and, wherein phenyl and heterocyclyl are optionally substituted with one, two, or three R¹² substituents;
R⁶ is hydrogen, deuterium, halo, C₁₋₆alkyl, halo-C₁₋₄alkyl, phenyl or morpholinyl;
wherein, when Q² is N, then R⁵ and R⁶ are other than halo; and,
wherein R⁵, R⁶ and R⁷ are not all simultaneously hydrogen;
Q³ is CR⁸ or N;
R⁸ is hydrogen or deuterium;
R⁹ is hydrogen, deuterium, halo, cyano, hydroxy, C₁₋₄alkyl, halo-C₁₋₄alkyl, C₂₋₄alkenyl, C₁₋₄alkylcarbonyl, amino, (C₁₋₄alkyl)amino, (C₁₋₄alkyl)₂amino, (C₁₋₄alkyl)₂amino(C₁₋₄alkyl)amino, C₁₋₄alkylthio, C₁₋₄alkyl-sulfonyl, amino-sulfonyl, C₁₋₄alkoxy, C₁₋₄alkoxy-C₁₋₄alkoxy, (C₁₋₄alkoxy)carbonyl, (C₁₋₄alkoxy)carbonylC₁₋₄alkyl, hydroxyC₁₋₄alkyl, hydroxyC₂₋₄alkenyl, hydroxyC₁₋₄alkylamino, hydroxyC₁₋₄alkoxyC₁₋₄alkyl, carboxyl_C₁₋₄alkyl, carboxyl_C₁₋₄alkylamino, carboxyl-C₁₋₄alkoxyC₁₋₄alkylamino, C₃₋₆cycloalkyl, heteroaryl or heterocyclyl, wherein heteroaryl is an aromatic 5-6 membered monocyclic ring having one, two, or three heteroatom ring members independently selected from N, O, or S; wherein heterocyclyl is a saturated or partially unsaturated 3-7 membered monocyclic, 6-10 membered bicyclic, 7-8 membered bicyclic, or 13-16 membered polycyclic ring system having one, two, or three heteroatom ring members independently selected from N, O, or S; and, wherein each heteroaryl or heterocyclyl is optionally substituted with one, two, or three R¹² substituents;
Q⁴ is CR¹⁰ or N;
R¹⁰ is hydrogen, deuterium, fluoro or hydroxy;
R¹¹ is hydrogen, deuterium, fluoro or hydroxy; and,
wherein R⁸, R⁹, R¹⁰ and R¹¹ are not simultaneously hydrogen; and,
R¹² is halo, hydroxy, C₁₋₆alkyl or C₃₋₆cycloalkyl; and,
wherein the form of the compound is selected from the group consisting of a salt, hydrate, solvate, racemate, enantiomer, diastereomer, stereoisomer, and tautomer thereof.

Another aspect of the compound of Formula (Ia1) includes a compound, wherein R² is selected from chloro, bromo, ethyl, methoxy, nitro, dimethyl-amino, 1H-pyrrol-1-yl, 1H-pyrazol-1-yl, 3-methyl-1H-pyrazol-1-yl, 4-methyl-1H-pyrazol-1-yl, 2H-1,2,3-triazol-2-yl, and 4-methyl-2H-1,2,3-triazol-2-yl.

Another aspect of the compound of Formula (Ia1) includes a compound, wherein R² is chloro, bromo, ethyl, methoxy, nitro, or dimethyl-amino.

Another aspect of the compound of Formula (Ia1) includes a compound, wherein R² is selected from chloro, bromo and 2H-1,2,3 triazol-2-yl.

Another aspect of the compound of Formula (Ia1) includes a compound, wherein R⁵ and R⁷ are independently selected from hydrogen, fluoro, chloro, bromo, cyano, methyl, ethyl, isopropyl, fluoromethyl, difluoromethyl, trifluoromethyl, trichloromethyl, hydroxy-methyl, methyl-thio, methoxy, butoxy, amino, N,N-dimethyl-amino, N-methyl-N-butyl-amino, N,N-dibutyl-amino, methoxy-carbonyl, butoxy-carbonyl, morpholinyl, 3,3-difluoro-azetindin-1-yl, 1-methylpiperazin-4-yl, and phenyl.

Another aspect of the compound of Formula (Ia1) includes a compound wherein R⁵ and R⁷ are independently selected from hydrogen, methyl and trifluoromethyl.

Another aspect of the compound of Formula (Ia1) includes a compound wherein R⁶ is hydrogen, fluoro, chloro, bromo, methyl or phenyl.

Another aspect of the compound of Formula (Ia1) includes a compound wherein R⁹ is hydrogen, fluoro, chloro, bromo, cyano, methyl, ethenyl, methyl-thio, methoxy, methoxyethoxy, hydroxy-methyl, methyl-carbonyl, N-(hydroxyethyl)amino, N[N,N-(dimethylamino)ethyl]amino, (carboxy-C₁₋₄alkyl)amino, (carboxy-C₁₋₄alkoxy-C₁₋₄alkyl)amino,

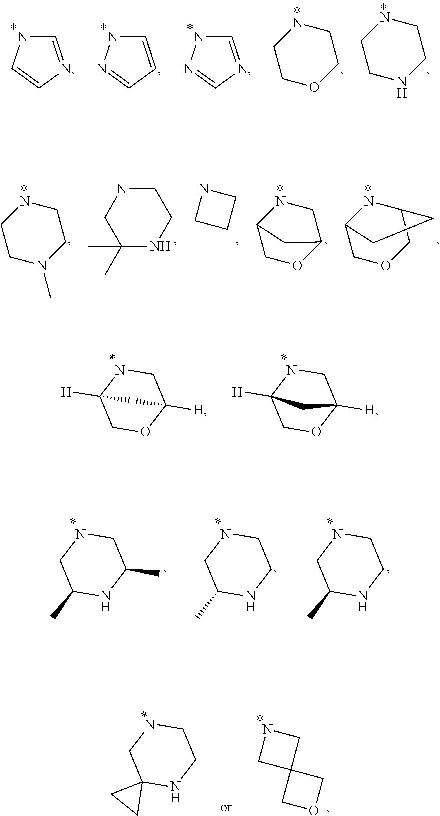

wherein the asterisk (*) designates the point of attachment.

Another aspect includes a compound of Formula (Ia1) wherein R⁹ is hydrogen, fluoro, chloro, bromo, cyano, methyl, isopropyl, trifluoromethyl, ethenyl, methylcarbonyl, amino, N-methylamino, N,N-dimethylamino, N—[(N,N-dimethylamino)ethyl]amino, methyl-thio, methyl-sulfonyl, amino-sulfonyl, methoxy, methoxy-ethoxy, methoxycarbonyl, hydroxymethyl, hydroxyethyl, hydroxypropyl, hydroxybutyl, 1-hydroxy-but-3-en-4-yl, N-(hydroxyethyl)amino, methoxy-carbonyl-ethyl, hydroxy-ethoxy-methyl, carboxy-ethyl, carboxy-propyl, N-(carboxy-ethyl)amino, N-(carboxy-propyl)amino, N-(carboxy-methoxy-ethyl)amino, cyclopropyl, 1H-imidazolyl, 1H-pyrazolyl, 1H-1,2,4-triazolyl, morpholinyl, piperazinyl, 4-methyl-piperazinyl, 3,3-dimethyl-piperazinyl, azetidinyl, 2-oxa-5-azabicyclo[2.2.1]heptanyl, 3-oxa-8-azabicyclo[3.2.1]octanyl, (1S,4S)-2-oxa-5-azabicyclo[2.2.1]heptanyl, (1R,4R)-2-oxa-5-azabicyclo[2.2.1]heptanyl, (3R,5S)-3,5-dimethylpiperazinyl, (3R)-3-methylpiperazinyl, (3S)-3-methylpiperazinyl, 4,7-diazaspiro[2.5]octanyl, or 2-oxa-6-azaspiro[3.3]heptanyl.

Another aspect of the compound of Formula (Ia1) includes a compound wherein R⁹ is selected from hydrogen, methyl, fluoro, chloro, bromo, methoxy, morpholinyl and 1-methyl-piperazin-4-yl, methyl-thio and hydroxy-methyl.

Another aspect of the compound of Formula (Ia1) includes a compound wherein R¹⁰ is hydrogen, fluoro, or hydroxy.

Another aspect of the compound of Formula (Ia1) includes a compound wherein R¹¹ is hydrogen, chloro, fluoro, or hydroxy.

Another aspect of the compound of Formula (Ia1) includes a compound wherein:
R² is chloro, bromo or 2H-1,2,3 triazol-2-yl;
Q² is CR⁶ or N;
R⁵ and R⁷ are independently selected from hydrogen, methyl and trifluoromethyl;
R⁶ is hydrogen, bromo or fluoro;
Q³ is CH or N;
R⁹ is selected from hydrogen, methyl, fluoro, chloro, methoxy, morpholinyl and 1-methyl-piperazin-4-yl, methyl-thio and hydroxy-methyl;
Q⁴ is CR¹⁰ or N;
R¹⁰ is hydrogen or fluoro; and,
R¹¹ is hydrogen or fluoro.

Another aspect of the compound of Formula (I) includes a compound of Formula (I) or a form thereof selected from a compound of Formula (Ia2):

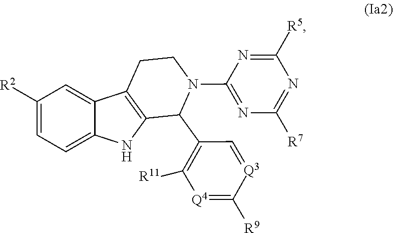

or a form thereof, wherein:
R² is halo, C₁₋₄alkyl, C₂₋₄alkenyl, nitro, C₁₋₄alkoxy, (C₁₋₄alkyl)₂amino, pyrrolyl, pyrazolyl, 3-methyl-pyrazolyl, 4-methyl-pyrazolyl, 2H-1,2,3-triazol-2-yl, or 4-methyl-2H-1,2,3-triazolyl-2-yl;
R⁵ and R⁷ are independently selected from hydrogen, deuterium, halo, cyano, C₁₋₄alkyl, halo-C₁₋₄alkyl, hydroxy-C₁₋₄alkyl, C₁₋₄alkyl-thio, C₁₋₄alkoxy, amino, (C₁₋₄alkyl)amino, (C₁₋₄alkyl)₂amino, C₁₋₄alkoxycarbonyl, phenyl or heterocyclyl, wherein heterocyclyl is a saturated or partially unsaturated 3-7 membered monocyclic ring system radical having one, two, or three heteroatom ring members independently selected from N, O, or S; and, wherein phenyl and heterocyclyl are optionally substituted with one, two, or three R¹² substituents;
Q³ is CR¹ or N;
R⁸ is hydrogen or deuterium;
R⁹ is hydrogen, deuterium, halo, cyano, hydroxy, C₁₋₄alkyl, halo-C₁₋₄alkyl, C₂₋₄alkenyl, C₁₋₄alkylcarbonyl, amino, (C₁₋₄alkyl)amino, (C₁₋₄alkyl)₂amino, (C₁₋₄alkyl)₂amino(C₁₋₄alkyl)amino, C₁₋₄alkylthio, C₁₋₄alkyl-sulfonyl, amino-sulfonyl, C₁₋₄alkoxy, C₁₋₄alkoxy-C₁₋₄alkoxy, (C₁₋₄alkoxy)carbonyl, (C₁₋₄alkoxy)carbonylC₁₋₄alkyl, hydroxyC₁₋₄alkyl, hydroxyC₂₋₄alkenyl, hydroxyC₁₋₄alkylamino, hydroxyC₁₋₄alkoxyC₁₋₄alkyl, carboxyl-C₁₋₄alkyl, carboxyl_C₁₋₄alkylamino, carboxyl_C₁₋₄alkoxy_C₁₋₄alkylamino, C₃₋₆cycloalkyl, heteroaryl or heterocyclyl, wherein heteroaryl is an aromatic 5-6 membered monocyclic ring having one, two, or three heteroatom ring members independently selected from N, O, or S; wherein heterocyclyl is a saturated or partially unsaturated 3-7 membered monocyclic, 6-10 membered bicyclic, 7-8 membered bicyclic, or 13-16 membered polycyclic ring system having one, two, or three heteroatom ring members independently selected from N, O, or S; and, wherein each heteroaryl or heterocyclyl is optionally substituted with one, two, or three R¹² substituents;
Q⁴ is CR¹⁰ or N;
R¹⁰ is hydrogen, deuterium, fluoro or hydroxy;
R¹¹ is hydrogen, deuterium, fluoro or hydroxy; and,
wherein R⁸, R⁹, R¹⁰ and R¹¹ are not simultaneously hydrogen; and,
R¹² is halo, hydroxy, C₁₋₆alkyl or C₃₋₆cycloalkyl; and,
wherein the form of the compound is selected from the group consisting of a salt, hydrate, solvate, racemate, enantiomer, diastereomer, stereoisomer, and tautomer thereof.

Another aspect of the compound of Formula (Ia2) includes a compound, wherein R² is selected from chloro, bromo, ethyl, methoxy, nitro, dimethyl-amino, 1H-pyrrol-1-yl, 1H-pyrazol-1-yl, 3-methyl-1H-pyrazol-1-yl, 4-methyl-1H-pyrazol-1-yl, 2H-1,2,3-triazol-2-yl, or 4-methyl-2H-1,2,3-triazol-2-yl.

Another aspect of the compound of Formula (Ia2) includes a compound, wherein R² is chloro, bromo, ethyl, methoxy, nitro or dimethyl-amino.

Another aspect of the compound of Formula (Ia2) includes a compound, wherein R² is selected from chloro, bromo or 2H-1,2,3 triazol-2-yl.

Another aspect of the compound of Formula (Ia2) includes a compound, wherein R⁵ and R⁷ are independently selected from hydrogen, fluoro, chloro, bromo, cyano, methyl, ethyl, isopropyl, fluoromethyl, difluoromethyl, trifluoromethyl, trichloromethyl, hydroxy-methyl, methyl-thio, methoxy, butoxy, amino, N,N-dimethyl-amino, N-methyl-N-butyl-amino, N,N-dibutyl-amino, methoxy-carbonyl, butoxy-carbonyl, morpholinyl, 3,3-difluoro-azetindin-1-yl, 1-methylpiperazin-4-yl, or phenyl.

Another aspect of the compound of Formula (Ia2) includes a compound wherein R⁵ and R⁷ are independently selected from hydrogen, methyl or trifluoromethyl.

Another aspect of the compound of Formula (Ia2) includes a compound wherein R⁶ is hydrogen, halo, methyl or phenyl.

Another aspect of the compound of Formula (Ia2) includes a compound wherein R⁹ is hydrogen, halo, cyano, methyl, ethenyl, methyl-thio, methoxy, methoxyethoxy, hydroxy-methyl, methyl-carbonyl, N-(hydroxyethyl)amino, N[N,N-(dimethylamino)ethyl]amino,

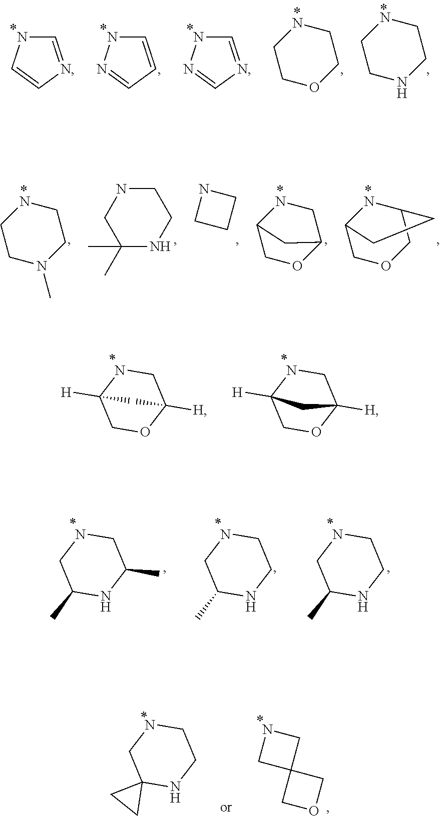

wherein the asterisk (*) designates the point of attachment.

Another aspect includes a compound of Formula (Ia2) wherein R⁹ is hydrogen, fluoro, chloro, bromo, cyano, methyl, isopropyl, trifluoromethyl, ethenyl, methylcarbonyl, amino, N-methylamino, N,N-dimethylamino, N—[(N,N-dimethylamino)ethyl]amino, methyl-thio, methyl-sulfonyl, amino-sulfonyl, methoxy, methoxy-ethoxy, methoxycarbonyl, hydroxymethyl, hydroxyethyl, hydroxypropyl, hydroxybutyl, 1-hydroxy-but-3-en-4-yl, N-(hydroxyethyl)amino, methoxy-carbonyl-ethyl, hydroxy-ethoxy-methyl, carboxy-ethyl, carboxy-propyl, N-(carboxy-ethyl)amino, N-(carboxy-propyl)amino, N-(carboxy-methoxy-ethyl)amino, cyclopropyl, 1H-imidazolyl, 1H-pyrazolyl, 1H-1,2,4-triazolyl, morpholinyl, piperazinyl, 4-methyl-piperazinyl, 3,3-dimethyl-piperazinyl, azetidinyl, 2-oxa-5-azabicyclo[2.2.1]heptanyl, 3-oxa-8-azabicyclo[3.2.1]octanyl, (1S,4S)-2-oxa-5-azabicyclo[2.2.1]heptanyl, (1R,4R)-2-oxa-5-azabicyclo[2.2.1]heptanyl, (3R,5S)-3,5-dimethylpiperazinyl, (3R)-3-methylpiperazinyl, (3S)-3-methylpiperazinyl, 4,7-diazaspiro[2.5]octanyl, or 2-oxa-6-azaspiro[3.3]heptanyl.

Another aspect of the compound of Formula (Ia2) includes a compound wherein R⁹ is selected from hydrogen, methyl, fluoro, chloro, bromo, methoxy, methyl-thio, hydroxy-methyl, morpholinyl or 1-methyl-piperazin-4-yl.

Another aspect of the compound of Formula (Ia2) includes a compound wherein R¹⁰ is hydrogen, fluoro, or hydroxy.

Another aspect of the compound of Formula (Ia2) includes a compound wherein R¹¹ is hydrogen, chloro, fluoro, or hydroxy.

Another aspect of the compound of Formula (Ia2) includes a compound wherein:
R² is chloro, bromo or 2H-1,2,3 triazol-2-yl;
Q² is CR⁶ or N;
R⁵ and R⁷ are independently selected from hydrogen, methyl or trifluoromethyl;
R⁶ is hydrogen, bromo or fluoro;
Q³ is CH or N;
R⁹ is selected from hydrogen, methyl, fluoro, chloro, bromo, methoxy, methyl-thio, hydroxy-methyl, morpholinyl or 1-methyl-piperazin-4-yl;
Q⁴ is CR¹⁰ or N;
R¹⁰ is hydrogen or fluoro; and,
R¹¹ is hydrogen or fluoro.

Another aspect of the compound of Formula (I) includes a compound of Formula (I) or a form thereof selected from Formula (Ib):

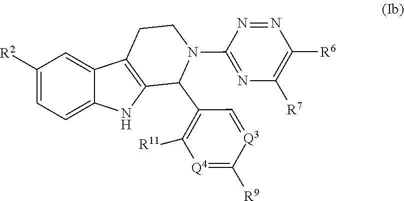

or a form thereof, wherein:
R² is halo, C₁₋₄alkyl, C₂₋₄alkenyl, nitro, C₁₋₄alkoxy, (C₁₋₄alkyl)₂amino, pyrrolyl, pyrazolyl, 3-methyl-pyrazolyl, 4-methyl-pyrazolyl, 2H-1,2,3-triazol-2-yl, or 4-methyl-2H-1,2,3-triazolyl-2-yl;
R⁶ is hydrogen, deuterium, halo, C₁₋₆alkyl, halo-C₁₋₄alkyl, phenyl or morpholinyl;
R⁷ is hydrogen, deuterium, halo, cyano, C₁₋₄alkyl, halo-C₁₋₄alkyl, hydroxy-C₁₋₄alkyl, C₁₋₄alkyl-thio, C₁₋₄alkoxy, amino, (C₁₋₄alkyl)amino, (C₁₋₄alkyl)₂amino, C₁₋₄alkoxycarbonyl, phenyl and heterocyclyl, wherein heterocyclyl is a saturated or partially unsaturated 3-7 membered monocyclic ring system radical having one, two, or three heteroatom ring members independently selected from N, O, or S; and, wherein phenyl and heterocyclyl are optionally substituted with one, two, or three R¹² substituents;
Q³ is CR⁸ or N;
R⁸ is hydrogen or deuterium;
R⁹ is hydrogen, deuterium, halo, cyano, hydroxy, C₁₋₄alkyl, halo-C₁₋₄alkyl, C₂₋₄alkenyl, C₁₋₄alkylcarbonyl, amino, (C₁₋₄alkyl)amino, (C₁₋₄alkyl)₂amino, (C₁₋₄alkyl)₂amino(C₁₋₄alkyl)amino, C₁₋₄alkylthio, C₁₋₄alkyl-sulfonyl, amino-sulfonyl, C₁₋₄alkoxy, C₁₋₄alkoxy-C₁₋₄alkoxy, (C₁₋₄alkoxy)carbonyl, (C₁₋₄alkoxy)carbonylC₁₋₄alkyl, hydroxyC₁₋₄alkyl, hydroxyC₂₋₄alkenyl, hydroxyC₁₋₄alkylamino, hydroxyC₁₋₄alkoxyC₁₋₄alkyl, carboxyl_C₁₋₄alkyl, carboxyl_C₁₋₄alkylamino, carboxyl_C₁₋₄alkoxy_C₁₋₄alkylamino, C₁₋₆cycloalkyl, heteroaryl or heterocyclyl, wherein heteroaryl is an aromatic 5-6 membered monocyclic ring having one, two, or three heteroatom ring members independently selected from N, O, or S; wherein heterocyclyl is a saturated or partially unsaturated 3-7 membered monocyclic, 6-10 membered bicyclic, 7-8 membered bicyclic, or 13-16 membered polycyclic ring system having one, two, or three heteroatom ring members independently selected from N, O, or S; and, wherein each heteroaryl or heterocyclyl is optionally substituted with one, two, or three R¹² substituents;
Q⁴ is CR¹⁰ or N;
R¹⁰ is hydrogen, deuterium, fluoro or hydroxy;
R¹¹ is hydrogen, deuterium, fluoro or hydroxy; and,
wherein R⁸, R⁹, R¹⁰ and R¹¹ are not simultaneously hydrogen; and,
R¹² is halo, hydroxy, C₁₋₆alkyl or C₃₋₆cycloalkyl, and,
wherein the form of the compound is selected from the group consisting of a salt, hydrate, solvate, racemate, enantiomer, diastereomer, stereoisomer, and tautomer thereof.

Another aspect of the compound of Formula (Ib) includes a compound wherein R² is chloro.

Another aspect of the compound of Formula (Ib) includes a compound wherein R⁶ is hydrogen or trifluoromethyl.

Another aspect of the compound of Formula (Ib) includes a compound wherein R⁷ is methyl or trifluoromethyl.

Another aspect of the compound of Formula (Ib) includes a compound wherein R⁹ is methyl or morpholinyl.

Another aspect of the compound of Formula (Ib) includes a compound wherein R¹⁰ is hydrogen or fluoro.

Another aspect of the compound of Formula (Ib) includes a compound wherein R¹¹ is hydrogen or fluoro.

Another aspect of the compound of Formula (Ib) includes a compound wherein.
R² is chloro;
R⁶ is hydrogen or trifluoromethyl;
R⁷ is methyl or trifluoromethyl;
Q³ is CH or N;
Q⁴ is CR¹⁰ or N;
R⁹ is methyl or morpholinyl;
R¹⁰ is hydrogen or fluoro; and
R¹¹ is hydrogen or fluoro.

Another aspect of the compound of Formula (I) includes a compound of Formula (I) or a form thereof selected from Formula (Ic):

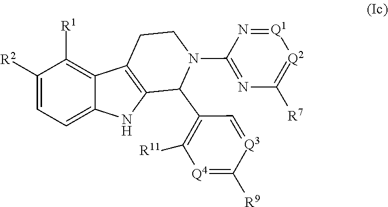

or a form thereof, wherein:
R¹ is hydrogen, deuterium, amino, nitro or fluoro;
R² is halo, C₁₋₄alkyl, C₂₋₄alkenyl, nitro, C₁₋₄alkoxy, (C₁₋₄alkyl)₂amino, pyrrolyl, pyrazolyl, 3-methyl-pyrazolyl, 4-methyl-pyrazolyl, 2H-1,2,3-triazol-2-yl, or 4-methyl-2H-1,2,3-triazolyl-2-yl;
Q¹ is CR⁵ or N;
Q² is CR⁶ or N;
wherein Q¹ and Q² are not simultaneously N;
R⁵ and R⁷ are independently selected from hydrogen, deuterium, halo, cyano, C₁₋₄alkyl, halo-C₁₋₄alkyl, hydroxy-C₁₋₄alkyl, C₁₋₄alkyl-thio, C₁₋₄alkoxy, amino, (C₁₋₄alkyl)amino, (C₁₋₄alkyl)₂amino, C₁₋₄alkoxycarbonyl, phenyl and heterocyclyl, wherein heterocyclyl is a saturated or partially unsaturated 3-7 membered monocyclic ring system radical having one, two, or three heteroatom ring members independently selected from N, O, or S; and, wherein phenyl and heterocyclyl are optionally substituted with one, two, or three R¹² substituents;
R⁶ is hydrogen, deuterium, halo, C₁₋₆alkyl, halo-C₁₋₄alkyl, phenyl or morpholinyl;
wherein, when Q² is N, then R⁵ and R⁶ are other than halo; and,
wherein R⁵, R⁶ and R⁷ are not all simultaneously hydrogen,
Q³ is CR⁸ or N;
R⁸ is hydrogen or deuterium;
R⁹ is hydrogen, deuterium, halo, cyano, hydroxy, C₁₋₄alkyl, halo-C₁₋₄alkyl, C₂₋₄alkenyl, C₁₋₄alkylcarbonyl, amino, (C₁₋₄alkyl)amino, (C₁₋₄alkyl)₂amino, (C₁₋₄alkyl)₂amino(C₁₋₄alkyl)amino, C₁₋₄alkylthio, C₁₋₄alkyl-sulfonyl, amino-sulfonyl, C₁₋₄alkoxy, C₁₋₄alkoxy-C₁₋₄alkoxy, (C₁₋₄alkoxy)carbonyl, (C₁₋₄alkoxy)carbonylC₁₋₄alkyl, hydroxyC₁₋₄alkyl, hydroxyC₂₋₄alkenyl, hydroxyC₁₋₄alkylamino, hydroxyC₁₋₄alkoxyC₁₋₄alkyl, carboxyl_C₁₋₄alkyl, carboxyl_C₁₋₄alkylamino, carboxyl_C₁₋₄alkoxy-C₁₋₄alkylamino, C₃₋₆cycloalkyl, heteroaryl or heterocyclyl, wherein heteroaryl is an aromatic 5-6 membered monocyclic ring having one, two, or three heteroatom ring members independently selected from N, O, or S; wherein heterocyclyl is a saturated or partially unsaturated 3-7 membered monocyclic, 6-10 membered bicyclic, 7-8 membered bicyclic, or 13-16 membered polycyclic ring system having one, two, or three heteroatom ring members independently selected from N, O, or S; and, wherein each heteroaryl or heterocyclyl is optionally substituted with one, two, or three R¹² substituents;
Q⁴ is CR¹⁰ or N;
R¹⁰ is hydrogen, deuterium, fluoro or hydroxy;
R¹¹ is hydrogen, deuterium, fluoro or hydroxy; and,
wherein R⁸, R⁹, R¹⁰ and R¹¹ are not simultaneously hydrogen; and,
R¹² is halo, hydroxy, C₁₋₆alkyl or C₃₋₆cycloalkyl; and,
wherein the form of the compound is selected from the group consisting of a salt, hydrate, solvate, racemate, enantiomer, diastereomer, stereoisomer, and tautomer thereof.

Another aspect of the compound of Formula (Ic) includes a compound wherein R¹ is fluoro.

Another aspect of the compound of Formula (Ic) includes a compound wherein R² is chloro, fluoro, or methyl.

Another aspect of the compound of Formula (Ic) includes a compound wherein Q¹ is CR⁵.

Another aspect of the compound of Formula (Ic) includes a compound wherein R⁵ and R⁷ are independently selected from hydrogen, chloro, methyl, trifluoromethyl, methoxy-carbonyl and cyano.

Another aspect of the compound of Formula (Ic) includes a compound wherein R⁶ is hydrogen, chloro, fluoro or methyl.

Another aspect of the compound of Formula (Ic) includes a compound wherein R⁹ is hydrogen, fluoro, choro, methyl, hydroxy-butyl, amino, N-methylamino, dimethylamino, cyano, trifluoromethyl, methyl-sulfonyl, amino-sulfonyl and 1-hydroxy-but-3-en-4-yl,

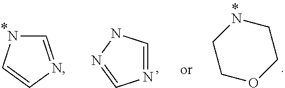

Another aspect of the compound of Formula (Ic) includes a compound wherein R⁹ is hydrogen, fluoro, choro, methyl, hydroxy-butyl, amino, N-methylamino, dimethylamino, cyano, trifluoromethyl, methyl-sulfonyl, amino-sulfonyl and 1-hydroxy-but-3-en-4-yl, 1H-imidazolyl, 1H-1,2,4-triazolyl or morpholinyl.

Another aspect of the compound of Formula (Ic) includes a compound wherein R¹⁰ is hydrogen or fluoro.

Another aspect of the compound of Formula (Ic) includes a compound wherein R¹¹ is hydrogen or fluoro.

Another aspect of the compound of Formula (I) includes a compound of Formula (I) or a form thereof selected from Formula (Id):

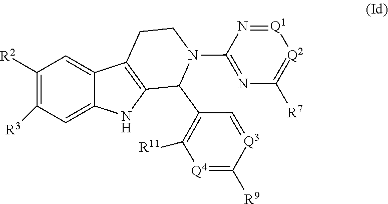

or a form thereof, wherein:
R² is halo, C₁₋₄alkyl, C₂₋₄alkenyl, nitro, C₁₋₄alkoxy, (C₁₋₄alkyl)₂amino, pyrrolyl, pyrazolyl, 3-methyl-pyrazolyl, 4-methyl-pyrazolyl, 2H-1,2,3-triazol-2-yl, or 4-methyl-2H-1,2,3-triazolyl-2-yl;
R³ is hydrogen, deuterium, amino or fluoro;
Q¹ is CR⁵ or N;
Q² is CR⁶ or N;
wherein Q¹ and Q² are not simultaneously N;
R⁵ and Rare independently selected from hydrogen, deuterium, halo, cyano, C₁₋₄alkyl, halo-C₁₋₄alkyl, hydroxy-C₁₋₄alkyl, C₁₋₄alkyl-thio, C₁₋₄alkoxy, amino, (C₁₋₄alkyl)amino, (C₁₋₄alkyl)₂amino, C₁₋₄alkoxycarbonyl, phenyl and heterocyclyl, wherein heterocyclyl is a saturated or partially unsaturated 3-7 membered monocyclic ring system radical having one, two, or three heteroatom ring members independently selected from N, O, or S; and, wherein phenyl and heterocyclyl are optionally substituted with one, two, or three R¹² substituents;
R⁶ is hydrogen, deuterium, halo, C₁₋₆alkyl, halo-C₁₋₄alkyl, phenyl or morpholinyl;
wherein, when Q² is N, then R⁵ and R⁶ are other than halo; and,
wherein R⁵, R⁶ and R⁷ are not all simultaneously hydrogen;
Q³ is CR⁸ or N;
R⁸ is hydrogen or deuterium;
R⁹ is hydrogen, deuterium, halo, cyano, hydroxy, C₁₋₄alkyl, halo-C₁₋₄alkyl, C₂₋₄alkenyl, C₁₋₄alkylcarbonyl, amino, (C₁₋₄alkyl)amino, (C₁₋₄alkyl)₂amino, (C₁₋₄alkyl)₂amino(C₁₋₄alkyl)amino, C₁₋₄alkylthio, C₁₋₄alkyl-sulfonyl, amino-sulfonyl, C₁₋₄alkoxy, C₁₋₄alkoxy-C₁₋₄alkoxy, (C₁₋₄alkoxy)carbonyl, (C₁₋₄alkoxy)carbonylC₁₋₄alkyl, hydroxyC₁₋₄alkyl, hydroxyC₂₋₄alkenyl, hydroxyC₁₋₄alkylamino, hydroxyC₁₋₄alkoxyC₁₋₄alkyl, carboxyl_C₁₋₄alkyl, carboxyl-C₁₋₄alkylamino, carboxyl_C₁₋₄alkoxy_C₁₋₄alkylamino, C₃₋₆cycloalkyl, heteroaryl or heterocyclyl, wherein heteroaryl is an aromatic 5-6 membered monocyclic ring having one, two, or three heteroatom ring members independently selected from N, O, or S; wherein heterocyclyl is a saturated or partially unsaturated 3-7 membered monocyclic, 6-10 membered bicyclic, 7-8 membered bicyclic, or 13-16 membered polycyclic ring system having one, two, or three heteroatom ring members independently selected from N, O, or S; and, wherein each heteroaryl or heterocyclyl is optionally substituted with one, two, or three R¹² substituents;
Q⁴ is CR¹⁰ or N;
R¹⁰ is hydrogen, deuterium, fluoro or hydroxy;
R¹¹ is hydrogen, deuterium, fluoro or hydroxy; and,
wherein R⁸, R⁹, R¹⁰ and R¹¹ are not simultaneously hydrogen; and,
R¹² is halo, hydroxy, C₁₋₆alkyl or C₃₋₆cycloalkyl; and,
wherein the form of the compound is selected from the group consisting of a salt, hydrate, solvate, racemate, enantiomer, diastereomer, stereoisomer, and tautomer thereof.

Another aspect of the compound of Formula (Id) includes a compound wherein R² is chloro.

Another aspect of the compound of Formula (Id) includes a compound wherein R³ is fluoro, hydroxy or amino.

Another aspect of the compound of Formula (Id) includes a compound wherein Q¹ is CR⁵.

Another aspect of the compound of Formula (Id) includes a compound wherein R⁵ and R⁷ are independently selected from hydrogen, chloro, bromo, methyl, ethyl, isopropyl, trifluoromethyl, cyano and N,N-dimethyl-amino.

Another aspect of the compound of Formula (Id) includes a compound wherein R⁶ is hydrogen, chloro, fluoro or trifluoromethyl.

Another aspect of the compound of Formula (Id) includes a compound wherein R⁸ is hydrogen or fluoro.

In another aspect of the compound of Formula (Id), R⁸ is not H when Q⁴ is N.

Another aspect of the compound of Formula (Id) includes a compound wherein R⁹ is hydrogen, halo, methyl, isopropyl, hydroxy-methyl, hydroxy-ethyl, hydroxy-methyl, hydroxy-propyl, ethenyl, methoxy, dimethylamino, cyano, trifluoromethyl, cyclopropyl, methyl-sulfonyl, amino-sulfonyl, methoxy-carbonyl-ethyl, hydroxy-ethoxy-methyl, 1-hydroxy-but-3-en-4-yl, or morpholinyl.

Another aspect of the compound of Formula (Id) includes a compound wherein R¹⁰ is hydrogen or fluoro.

Another aspect of the compound of Formula (Id) includes a compound wherein R¹¹ is hydrogen or fluoro.

Another aspect of the compound of Formula (I) includes a compound of Formula (I) or a form thereof selected from Formula (Ie):

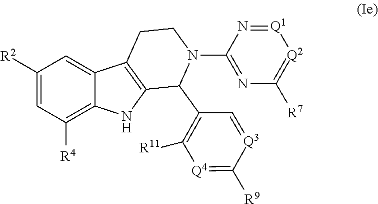

or a form thereof, wherein:
R² is halo, C₁₋₄alkyl, C₂₋₄alkenyl, nitro, C₁₋₄alkoxy, (C₁₋₄alkyl)₂amino, pyrrolyl, pyrazolyl, 3-methyl-pyrazolyl, 4-methyl-pyrazolyl, 2H-1,2,3-triazol-2-yl, or 4-methyl-2H-1,2,3-triazolyl-2-yl;
R³ is hydrogen, deuterium, amino or fluoro;
R⁴ is hydrogen, amino or hydroxy;
Q¹ is CR⁵ or N;
Q² is CR⁶ or N;
wherein Q¹ and Q² are not simultaneously N;
R⁵ and R⁷ are independently selected from hydrogen, deuterium, halo, cyano, C₁₋₄alkyl, halo-C₁₋₄alkyl, hydroxy-C₁₋₄alkyl, C₁₋₄alkyl-thio, C₁₋₄alkoxy, amino, (C₁₋₄alkyl)amino, (C₁₋₄alkyl)₂amino, C₁₋₄alkoxycarbonyl, phenyl and heterocyclyl, wherein heterocyclyl is a saturated or partially unsaturated 3-7 membered monocyclic ring system radical having one, two, or three heteroatom ring members independently selected from N, O, or S; and, wherein phenyl and heterocyclyl are optionally substituted with one, two, or three R¹² substituents;
R⁶ is hydrogen, deuterium, halo, C₁₋₆alkyl, halo-C₁₋₄alkyl, phenyl or morpholinyl;
wherein, when Q² is N, then R⁵ and R⁶ are other than halo; and,
wherein R⁵, R⁶ and R⁷ are not all simultaneously hydrogen,
Q³ is CR⁸ or N;
R⁸ is hydrogen or deuterium;
R⁹ is hydrogen, deuterium, halo, cyano, hydroxy, C₁₋₄alkyl, halo-C₁₋₄alkyl, C₂₋₄alkenyl, C₁₋₄alkylcarbonyl, amino, (C₁₋₄alkyl)amino, (C₁₋₄alkyl)₂amino, (C₁₋₄alkyl)₂amino(C₁₋₄alkyl)amino, C₁₋₄alkylthio, C₁₋₄alkyl-sulfonyl, amino-sulfonyl, C₁₋₄alkoxy, C₁₋₄alkoxy-C₁₋₄alkoxy, (C₁₋₄alkoxy)carbonyl, (C₁₋₄alkoxy)carbonylC₁₋₄alkyl, hydroxyC₁₋₄alkyl, hydroxyC₂₋₄alkenyl, hydroxyC₁₋₄alkylamino, hydroxyC₁₋₄alkoxyC₁₋₄alkyl, carboxyl-C₁₋₄alkyl, carboxyl_C₁₋₄alkylamino, carboxyl C₁₋₄alkoxy_C₁₋₄alkylamino, C₃₋₆cycloalkyl, heteroaryl or heterocyclyl, wherein heteroaryl is an aromatic 5-6 membered monocyclic ring having one, two, or three heteroatom ring members independently selected from N, O, or S; wherein heterocyclyl is a saturated or partially unsaturated 3-7 membered monocyclic, 6-10 membered bicyclic, 7-8 membered bicyclic, or 13-16 membered polycyclic ring system having one, two, or three heteroatom ring members independently selected from N, O, or S; and, wherein each heteroaryl or heterocyclyl is optionally substituted with one, two, or three R¹² substituents;
Q⁴ is CR¹⁰ or N;
R¹⁰ is hydrogen, deuterium, fluoro or hydroxy;
R¹¹ is hydrogen, deuterium, fluoro or hydroxy; and,
wherein R⁸, R⁹, R¹⁰ and R¹¹ are not simultaneously hydrogen; and,
R¹¹ is halo, hydroxy, C₁₋₆alkyl or C₃₋₆cycloalkyl; and,
wherein the form of the compound is selected from the group consisting of a salt, hydrate, solvate, racemate, enantiomer, diastereomer, stereoisomer, and tautomer thereof.

Another aspect of the compound of Formula (Ie) includes a compound wherein R² is chloro.

Another aspect of the compound of Formula (Ie) includes a compound wherein R⁴ is hydrogen, fluoro, hydroxy or amino.

Another aspect of the compound of Formula (Ie) includes a compound wherein Q¹ is CR⁵.

Another aspect of the compound of Formula (Ie) includes a compound wherein R⁵ and R⁷ are independently selected from hydrogen, methyl and trifluoromethyl.

Another aspect of the compound of Formula (Ie) includes a compound wherein R⁶ is hydrogen or fluoro.

Another aspect of the compound of Formula (Ie) includes a compound wherein R⁹ is methyl, cyano or morpholinyl.

Another aspect of the compound of Formula (Ie) includes a compound wherein R¹⁰ is hydrogen or fluoro.

Another aspect of the compound of Formula (Ie) includes a compound wherein R¹¹ is hydrogen or fluoro.

An aspect of the compound of Formula (I) or a form thereof includes a compound selected from the group consisting of:

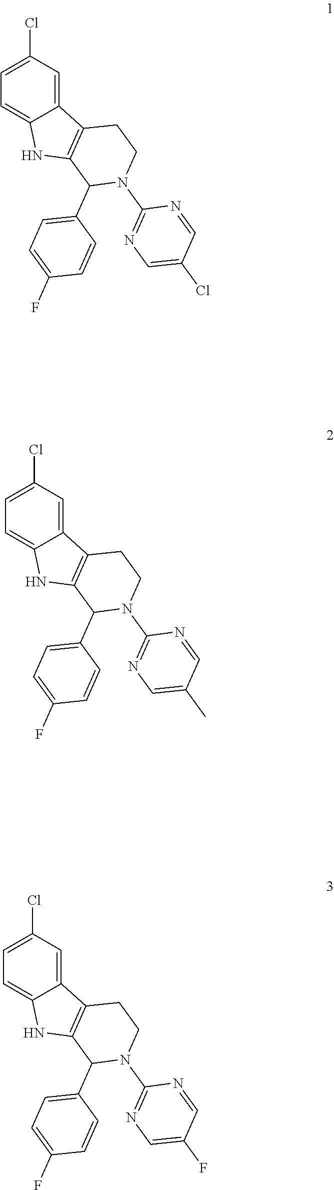 [SMILES: Fc1ccc(C2c3[nH]c4ccc(Cl)cc4c3CCN2c2ncc(Cl)cn2)cc1 ; Cc1cnc(N2CCc3c([nH]c4ccc(Cl)cc34)C2c2ccc(F)cc2)nc1 ; Fc1ccc(C2c3[nH]c4ccc(Cl)cc4c3CCN2c2ncc(F)cn2)cc1]

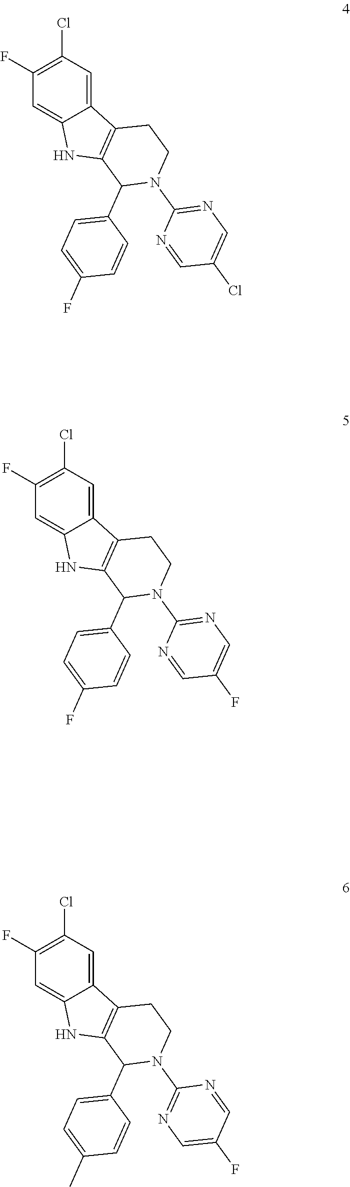 [SMILES: Fc1ccc(C2c3[nH]c4cc(F)c(Cl)cc4c3CCN2c2ncc(Cl)cn2)cc1 ; Fc1ccc(C2c3[nH]c4cc(F)c(Cl)cc4c3CCN2c2ncc(F)cn2)cc1 ; Cc1ccc(C2c3[nH]c4cc(F)c(Cl)cc4c3CCN2c2ncc(F)cn2)cc1]

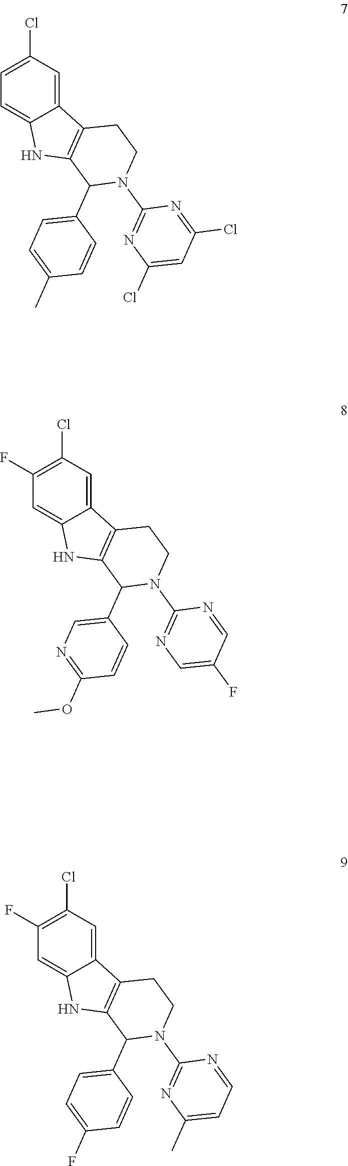 [SMILES: Cc1ccc(C2c3[nH]c4ccc(Cl)cc4c3CCN2c2nc(Cl)cc(Cl)n2)cc1 ; COc1ccc(C2c3[nH]c4cc(F)c(Cl)cc4c3CCN2c2ncc(F)cn2)cn1 ; Cc1ccnc(N2CCc3c([nH]c4cc(F)c(Cl)cc34)C2c2ccc(F)cc2)n1]

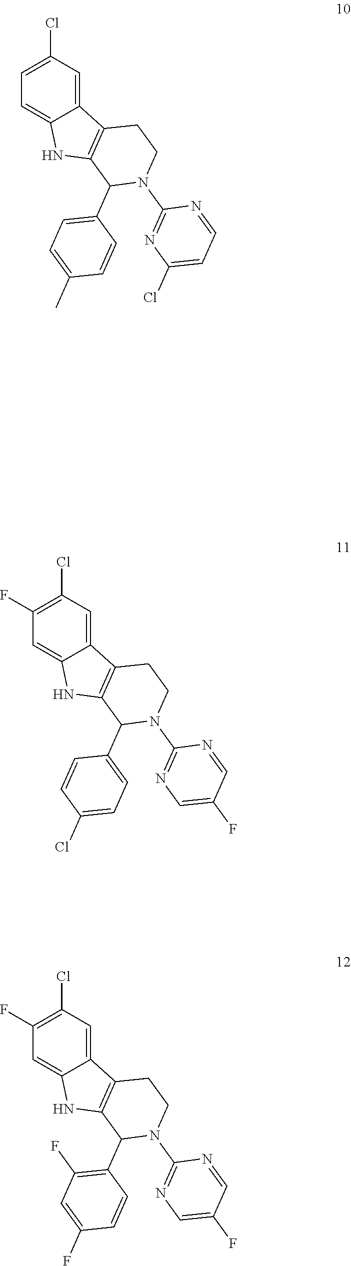 [SMILES: Cc1ccc(C2c3[nH]c4ccc(Cl)cc4c3CCN2c2nccc(Cl)n2)cc1 ; Fc1cnc(N2CCc3c([nH]c4cc(F)c(Cl)cc34)C2c2ccc(Cl)cc2)nc1 ; Fc1cnc(N2CCc3c([nH]c4cc(F)c(Cl)cc34)C2c2ccc(F)cc2F)nc1]

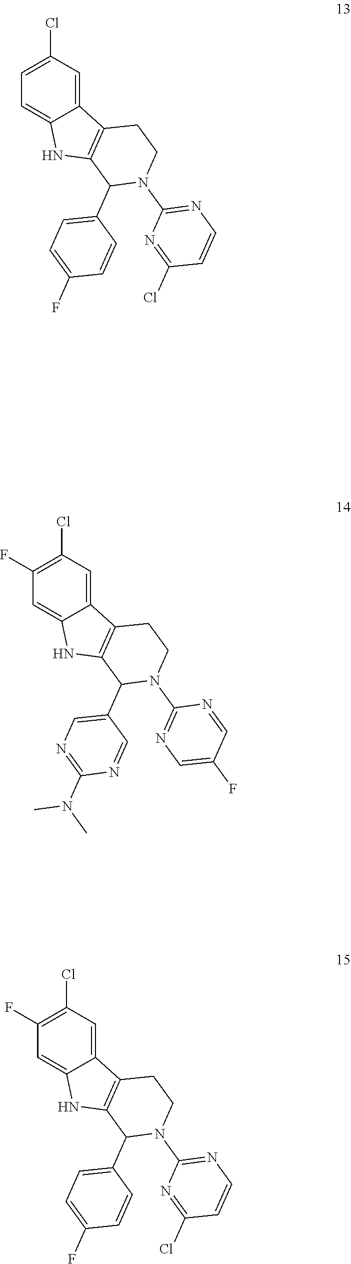

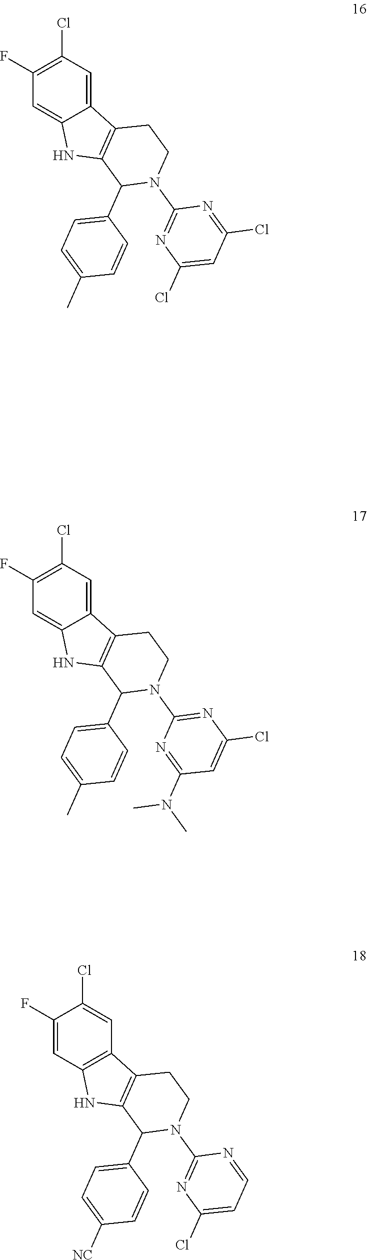 [SMILES: Cc1ccc(C2c3[nH]c4cc(F)c(Cl)cc4c3CCN2c2nc(Cl)cc(Cl)n2)cc1 ; Cc1ccc(C2c3[nH]c4cc(F)c(Cl)cc4c3CCN2c2nc(Cl)cc(N(C)C)n2)cc1 ; N#Cc1ccc(C2c3[nH]c4cc(F)c(Cl)cc4c3CCN2c2nccc(Cl)n2)cc1]

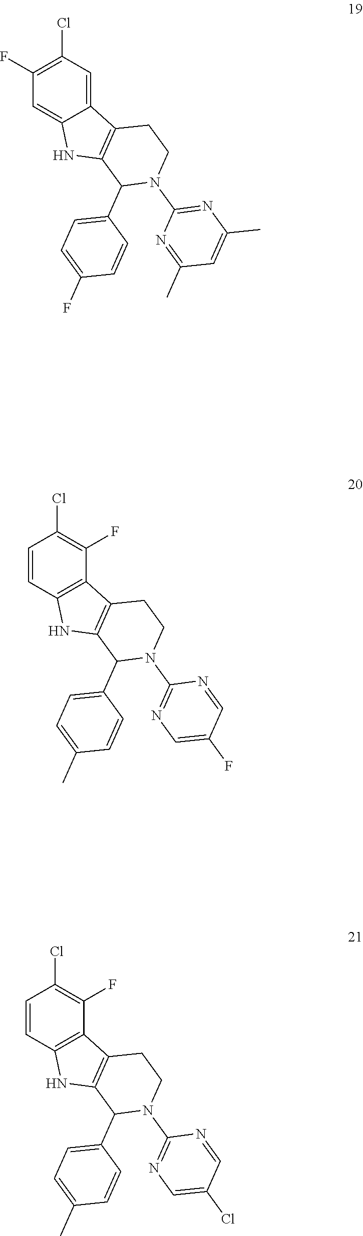 [SMILES: Cc1cc(C)nc(N2CCc3c([nH]c4cc(F)c(Cl)cc34)C2c2ccc(F)cc2)n1 ; Cc1ccc(C2c3[nH]c4ccc(Cl)c(F)c4c3CCN2c2ncc(F)cn2)cc1 ; Cc1ccc(C2c3[nH]c4ccc(Cl)c(F)c4c3CCN2c2ncc(Cl)cn2)cc1]

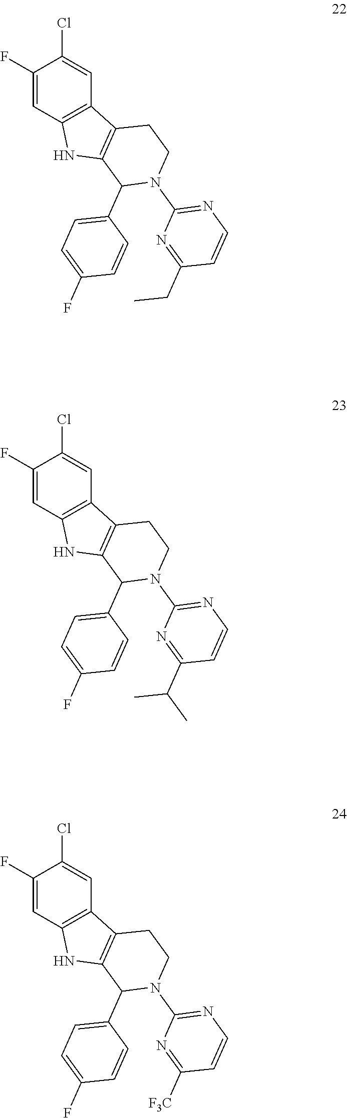 [SMILES: CCc1ccnc(N2CCc3c([nH]c4cc(F)c(Cl)cc34)C2c2ccc(F)cc2)n1 ; CC(C)c1ccnc(N2CCc3c([nH]c4cc(F)c(Cl)cc34)C2c2ccc(F)cc2)n1 ; Fc1ccc(C2c3[nH]c4cc(F)c(Cl)cc4c3CCN2c2nccc(C(F)(F)F)n2)cc1]

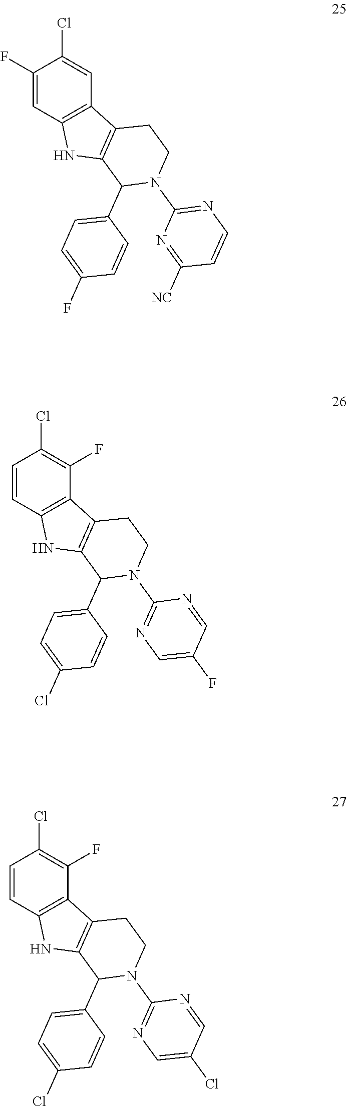 [SMILES: N#Cc1ccnc(N2CCc3c([nH]c4cc(F)c(Cl)cc34)C2c2ccc(F)cc2)n1 ; Fc1cnc(N2CCc3c([nH]c4ccc(Cl)c(F)c34)C2c2ccc(Cl)cc2)nc1 ; Fc1c(Cl)ccc2[nH]c3c(c12)CCN(c1ncc(Cl)cn1)C3c1ccc(Cl)cc1]

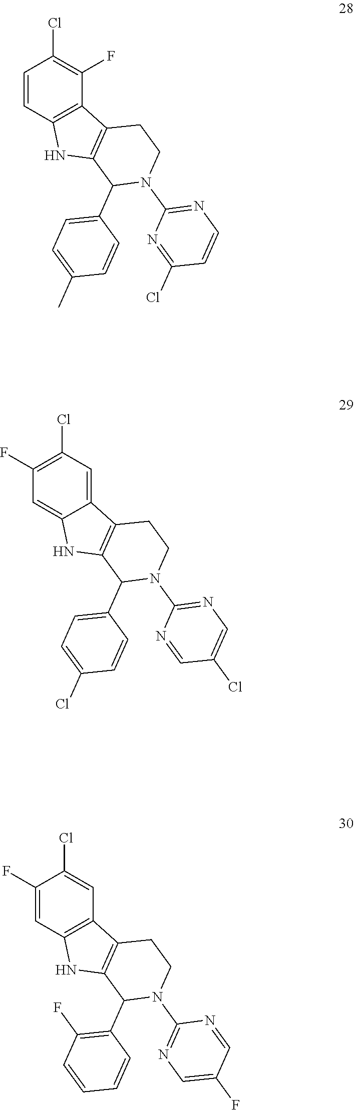

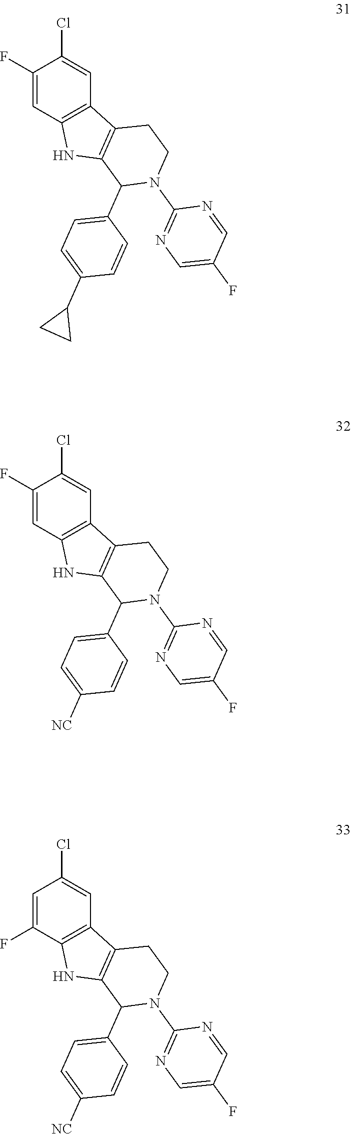 [SMILES: Fc1cnc(N2CCc3c([nH]c4cc(F)c(Cl)cc34)C2c2ccc(C3CC3)cc2)nc1 ; N#Cc1ccc(C2c3[nH]c4cc(F)c(Cl)cc4c3CCN2c2ncc(F)cn2)cc1 ; N#Cc1ccc(C2c3[nH]c4c(F)cc(Cl)cc4c3CCN2c2ncc(F)cn2)cc1]

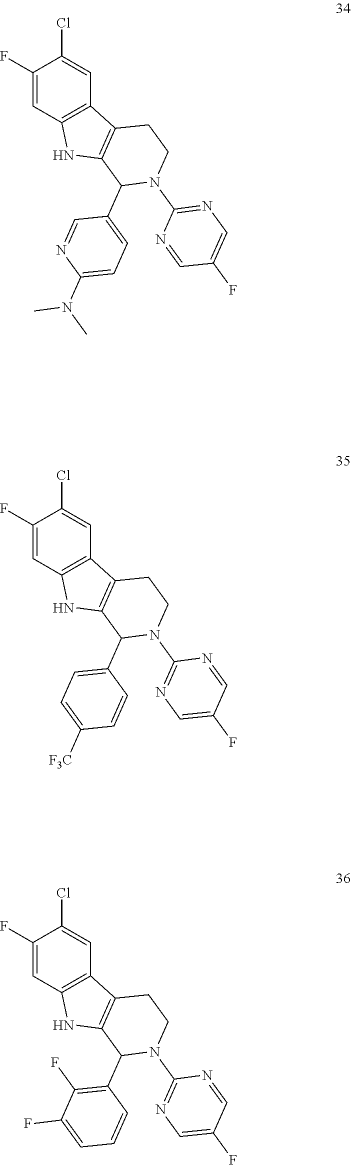 [SMILES: CN(C)c1ccc(C2c3[nH]c4cc(F)c(Cl)cc4c3CCN2c2ncc(F)cn2)cn1 ; Fc1cnc(N2CCc3c([nH]c4cc(F)c(Cl)cc34)C2c2ccc(C(F)(F)F)cc2)nc1 ; Fc1cnc(N2CCc3c([nH]c4cc(F)c(Cl)cc34)C2c2cccc(F)c2F)nc1]

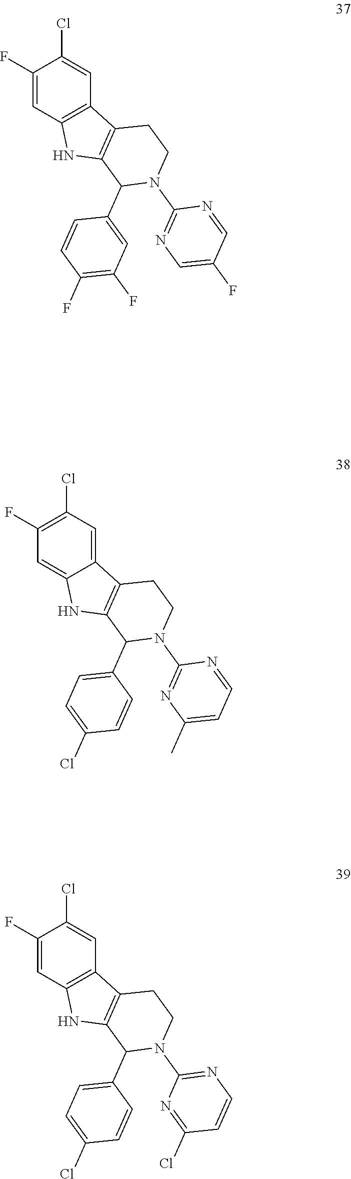 [SMILES: Fc1cnc(N2CCc3c([nH]c4cc(F)c(Cl)cc34)C2c2ccc(F)c(F)c2)nc1 ; Cc1ccnc(N2CCc3c([nH]c4cc(F)c(Cl)cc34)C2c2ccc(Cl)cc2)n1 ; Fc1cc2[nH]c3c(c2cc1Cl)CCN(c1nccc(Cl)n1)C3c1ccc(Cl)cc1]

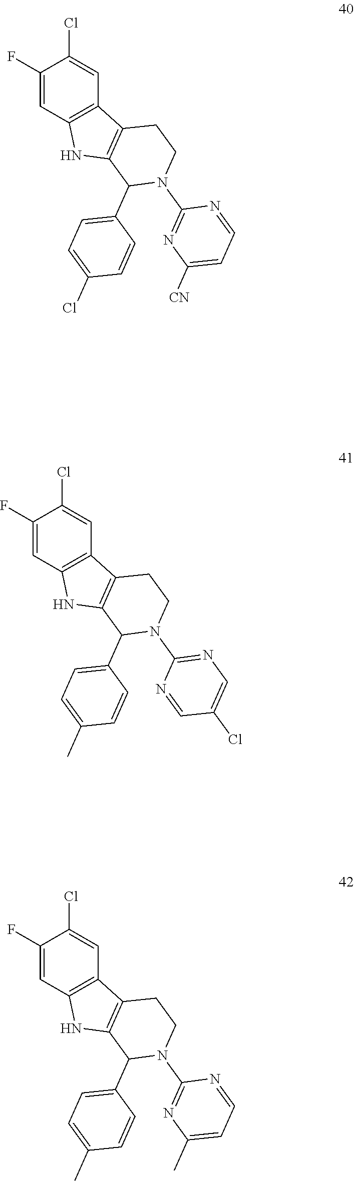 [SMILES: N#Cc1ccnc(N2CCc3c([nH]c4cc(F)c(Cl)cc34)C2c2ccc(Cl)cc2)n1 ; Cc1ccc(C2c3[nH]c4cc(F)c(Cl)cc4c3CCN2c2ncc(Cl)cn2)cc1 ; Cc1ccc(C2c3[nH]c4cc(F)c(Cl)cc4c3CCN2c2nccc(C)n2)cc1]

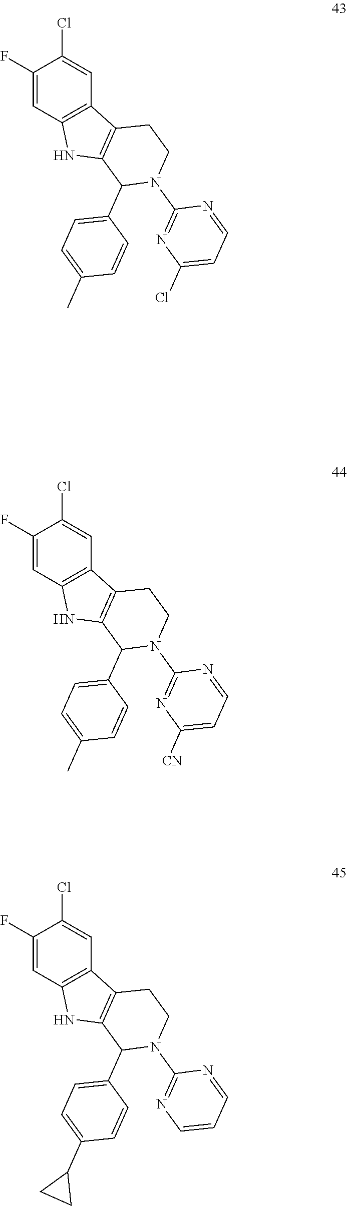 [SMILES: Cc1ccc(C2c3[nH]c4cc(F)c(Cl)cc4c3CCN2c2nccc(Cl)n2)cc1 ; Cc1ccc(C2c3[nH]c4cc(F)c(Cl)cc4c3CCN2c2nccc(C#N)n2)cc1 ; Fc1cc2[nH]c3c(c2cc1Cl)CCN(c1ncccn1)C3c1ccc(C2CC2)cc1]

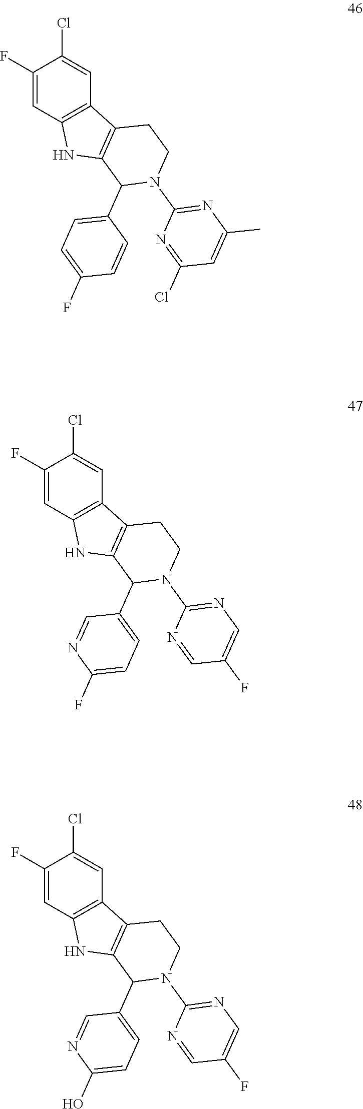 [SMILES: Cc1cc(Cl)nc(N2CCc3c([nH]c4cc(F)c(Cl)cc34)C2c2ccc(F)cc2)n1 ; Fc1cnc(N2CCc3c([nH]c4cc(F)c(Cl)cc34)C2c2ccc(F)nc2)nc1 ; Oc1ccc(C2c3[nH]c4cc(F)c(Cl)cc4c3CCN2c2ncc(F)cn2)cn1]

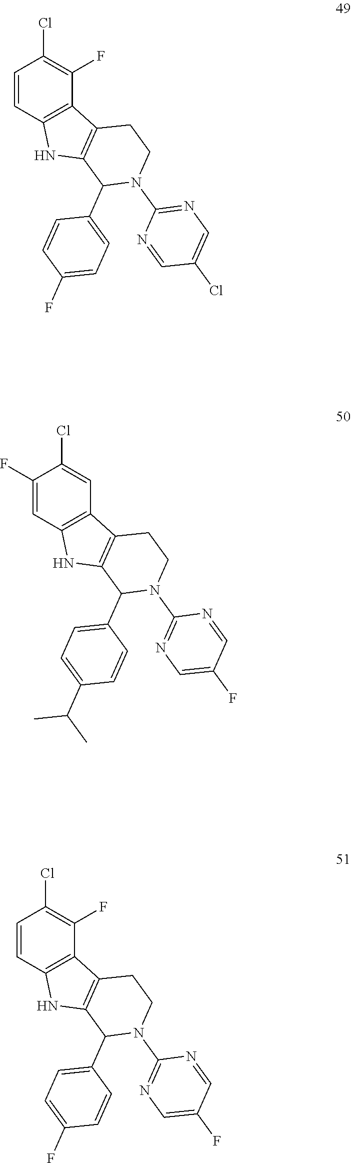 [SMILES: Fc1ccc(C2c3[nH]c4ccc(Cl)c(F)c4c3CCN2c2ncc(Cl)cn2)cc1 ; CC(C)c1ccc(C2c3[nH]c4cc(F)c(Cl)cc4c3CCN2c2ncc(F)cn2)cc1 ; Fc1ccc(C2c3[nH]c4ccc(Cl)c(F)c4c3CCN2c2ncc(F)cn2)cc1]

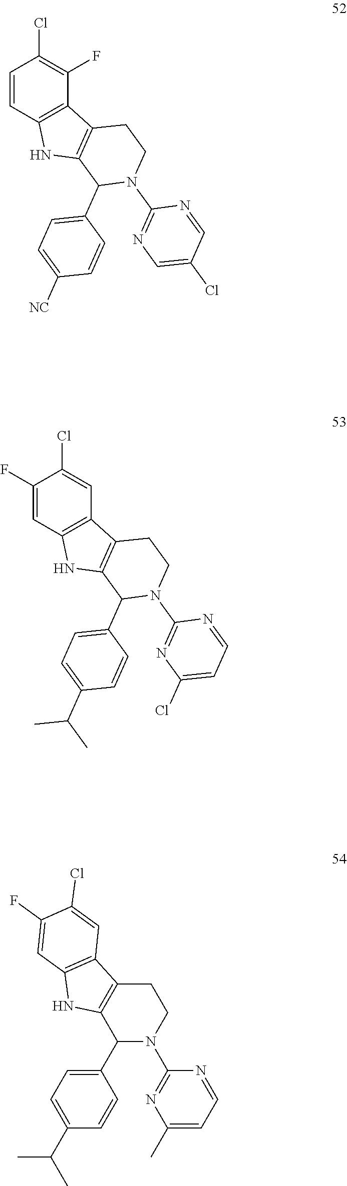 [SMILES: N#Cc1ccc(C2c3[nH]c4ccc(Cl)c(F)c4c3CCN2c2ncc(Cl)cn2)cc1 ; CC(C)c1ccc(C2c3[nH]c4cc(F)c(Cl)cc4c3CCN2c2nccc(Cl)n2)cc1 ; Cc1ccnc(N2CCc3c([nH]c4cc(F)c(Cl)cc34)C2c2ccc(C(C)C)cc2)n1]

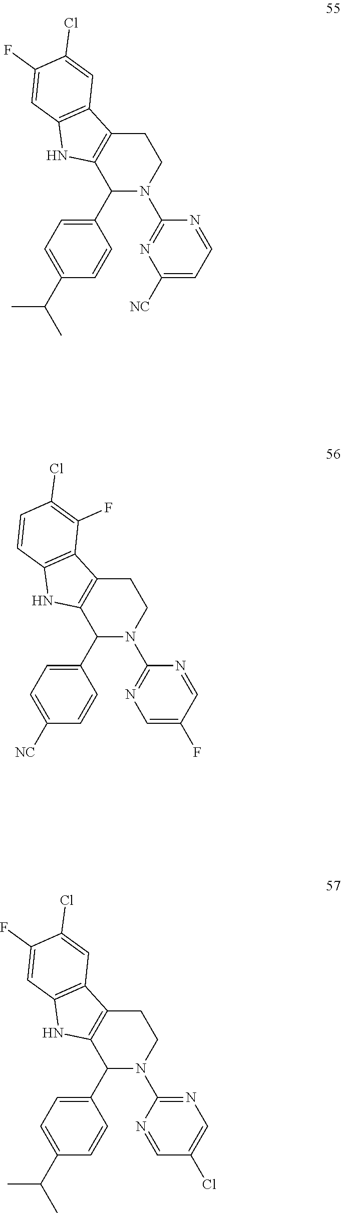 [SMILES: CC(C)c1ccc(C2c3[nH]c4cc(F)c(Cl)cc4c3CCN2c2nccc(C#N)n2)cc1 ; N#Cc1ccc(C2c3[nH]c4ccc(Cl)c(F)c4c3CCN2c2ncc(F)cn2)cc1 ; CC(C)c1ccc(C2c3[nH]c4cc(F)c(Cl)cc4c3CCN2c2ncc(Cl)cn2)cc1]

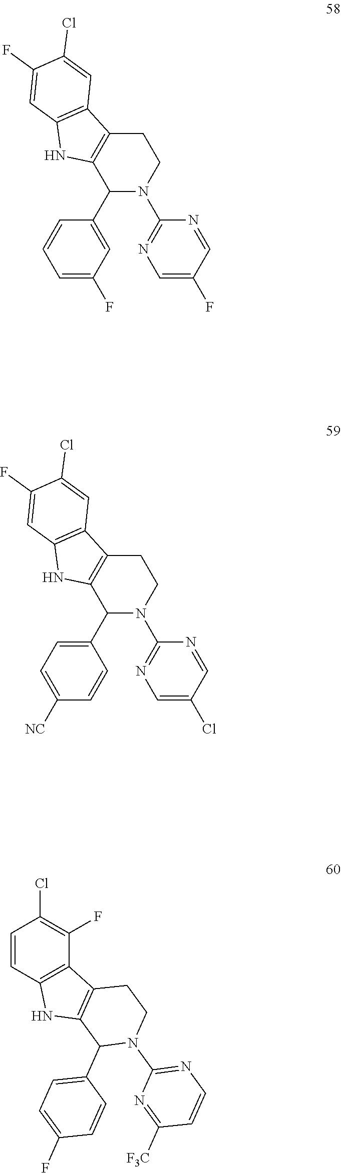

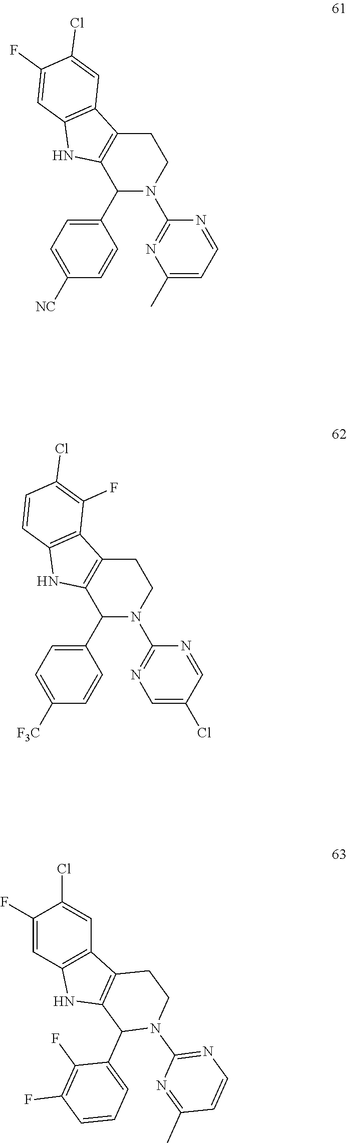 [SMILES: Cc1ccnc(N2CCc3c([nH]c4cc(F)c(Cl)cc34)C2c2ccc(C#N)cc2)n1 ; Fc1c(Cl)ccc2[nH]c3c(c12)CCN(c1ncc(Cl)cn1)C3c1ccc(C(F)(F)F)cc1 ; Cc1ccnc(N2CCc3c([nH]c4cc(F)c(Cl)cc34)C2c2cccc(F)c2F)n1]

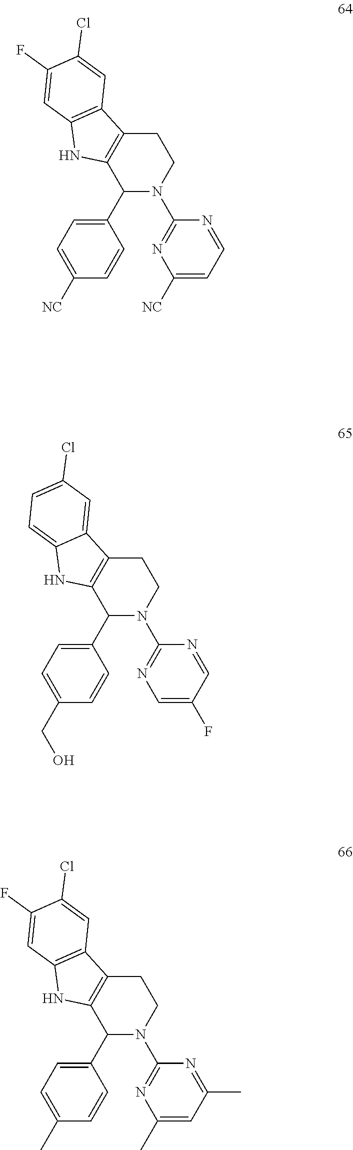 [SMILES: N#Cc1ccc(C2c3[nH]c4cc(F)c(Cl)cc4c3CCN2c2nccc(C#N)n2)cc1 ; OCc1ccc(C2c3[nH]c4ccc(Cl)cc4c3CCN2c2ncc(F)cn2)cc1 ; Cc1ccc(C2c3[nH]c4cc(F)c(Cl)cc4c3CCN2c2nc(C)cc(C)n2)cc1]

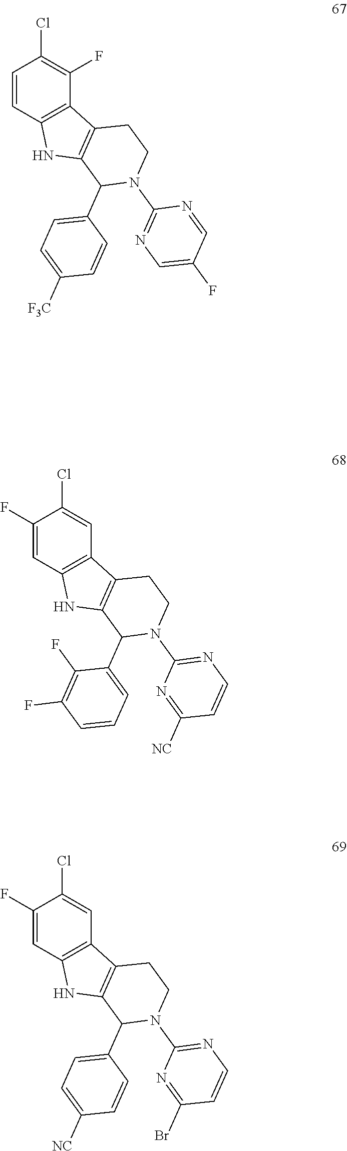 [SMILES: Fc1cnc(N2CCc3c([nH]c4ccc(Cl)c(F)c34)C2c2ccc(C(F)(F)F)cc2)nc1 ; N#Cc1ccnc(N2CCc3c([nH]c4cc(F)c(Cl)cc34)C2c2cccc(F)c2F)n1 ; N#Cc1ccc(C2c3[nH]c4cc(F)c(Cl)cc4c3CCN2c2nccc(Br)n2)cc1]

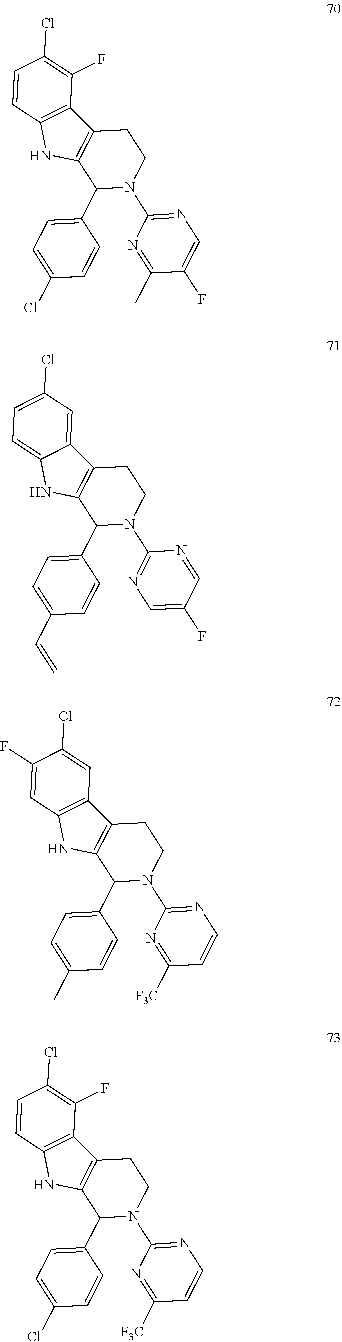

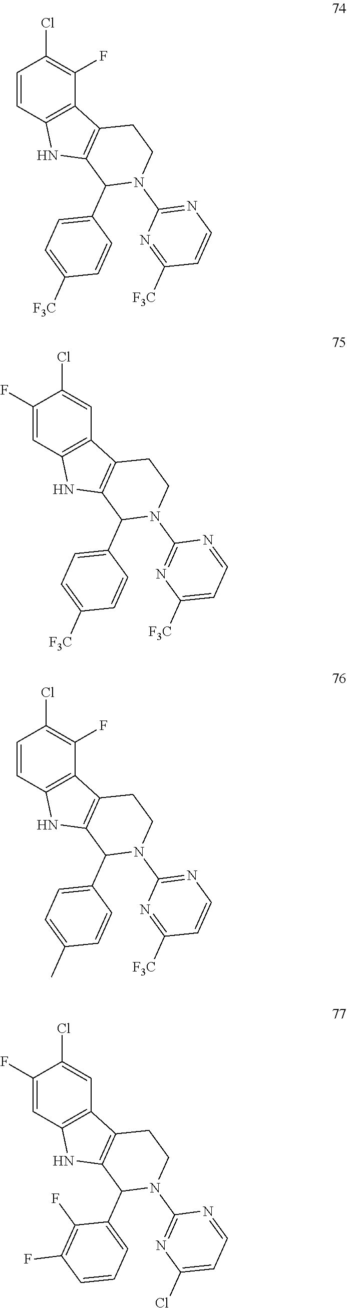 [SMILES: Fc1c(Cl)ccc2[nH]c3c(c12)CCN(c1nccc(C(F)(F)F)n1)C3c1ccc(C(F)(F)F)cc1 ; Fc1cc2[nH]c3c(c2cc1Cl)CCN(c1nccc(C(F)(F)F)n1)C3c1ccc(C(F)(F)F)cc1 ; Cc1ccc(C2c3[nH]c4ccc(Cl)c(F)c4c3CCN2c2nccc(C(F)(F)F)n2)cc1 ; Fc1cc2[nH]c3c(c2cc1Cl)CCN(c1nccc(Cl)n1)C3c1cccc(F)c1F]

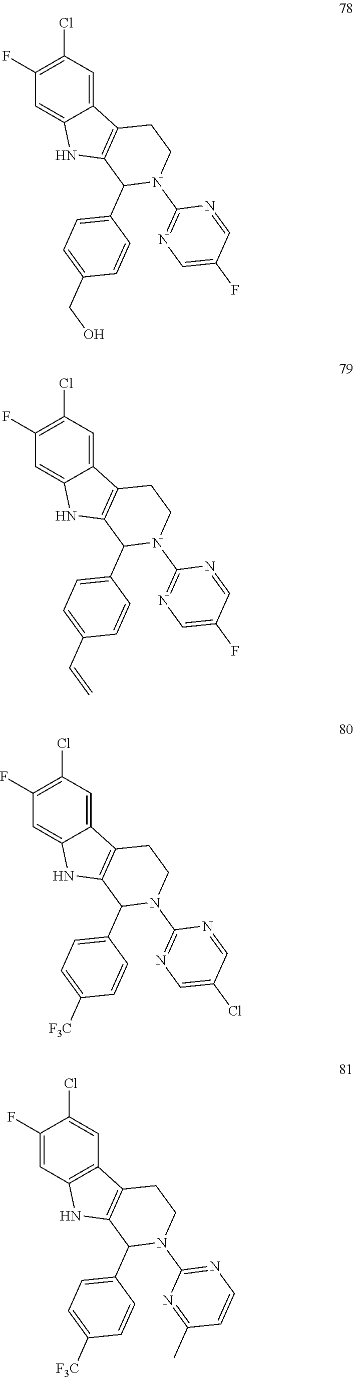 [SMILES: OCc1ccc(C2c3[nH]c4cc(F)c(Cl)cc4c3CCN2c2ncc(F)cn2)cc1 ; C=Cc1ccc(C2c3[nH]c4cc(F)c(Cl)cc4c3CCN2c2ncc(F)cn2)cc1 ; Fc1cc2[nH]c3c(c2cc1Cl)CCN(c1ncc(Cl)cn1)C3c1ccc(C(F)(F)F)cc1 ; Cc1ccnc(N2CCc3c([nH]c4cc(F)c(Cl)cc34)C2c2ccc(C(F)(F)F)cc2)n1]

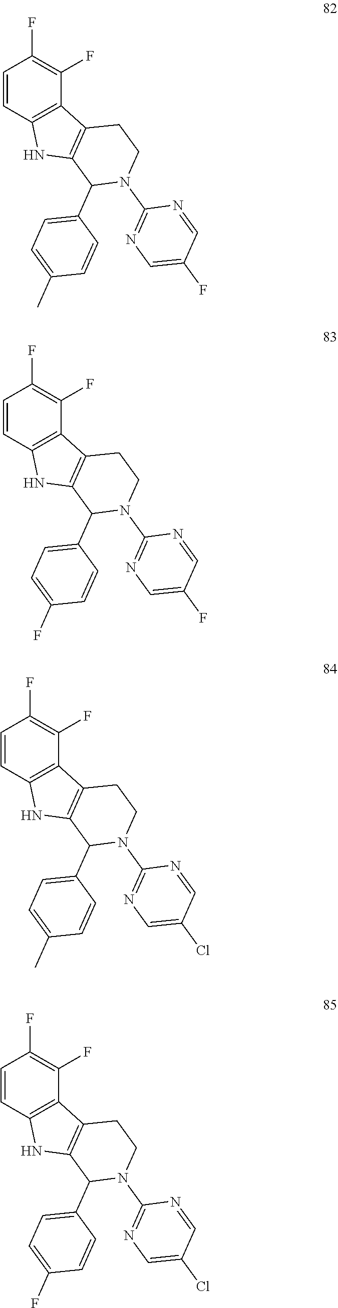

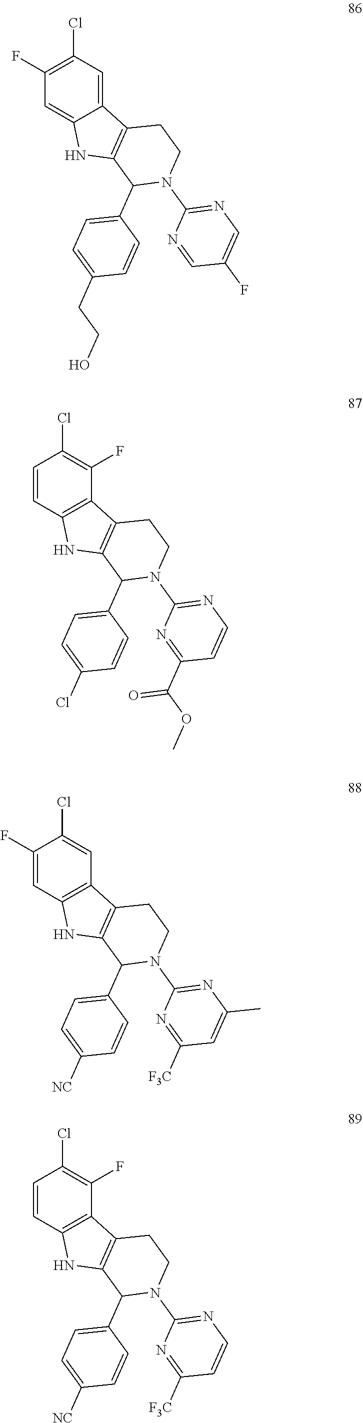

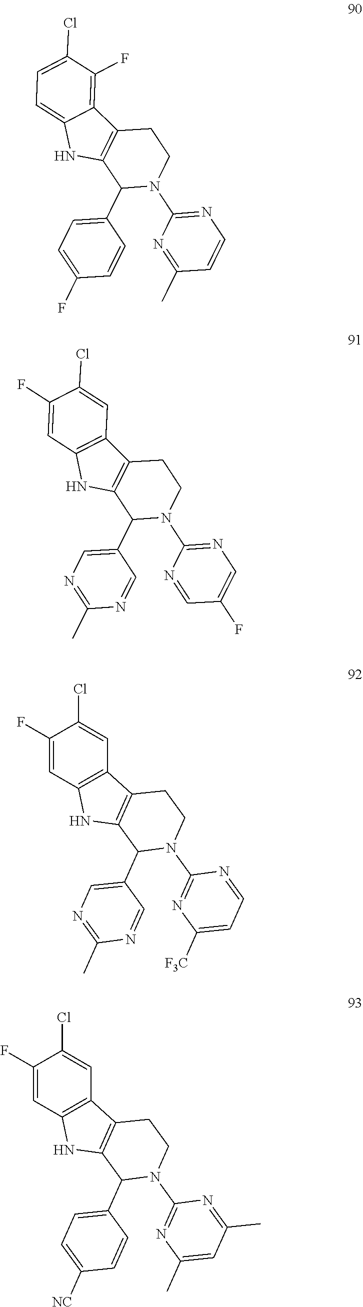 [SMILES: Cc1ccnc(N2CCc3c([nH]c4ccc(Cl)c(F)c34)C2c2ccc(F)cc2)n1 ; Cc1ncc(C2c3[nH]c4cc(F)c(Cl)cc4c3CCN2c2ncc(F)cn2)cn1 ; Cc1ncc(C2c3[nH]c4cc(F)c(Cl)cc4c3CCN2c2nccc(C(F)(F)F)n2)cn1 ; Cc1cc(C)nc(N2CCc3c([nH]c4cc(F)c(Cl)cc34)C2c2ccc(C#N)cc2)n1]

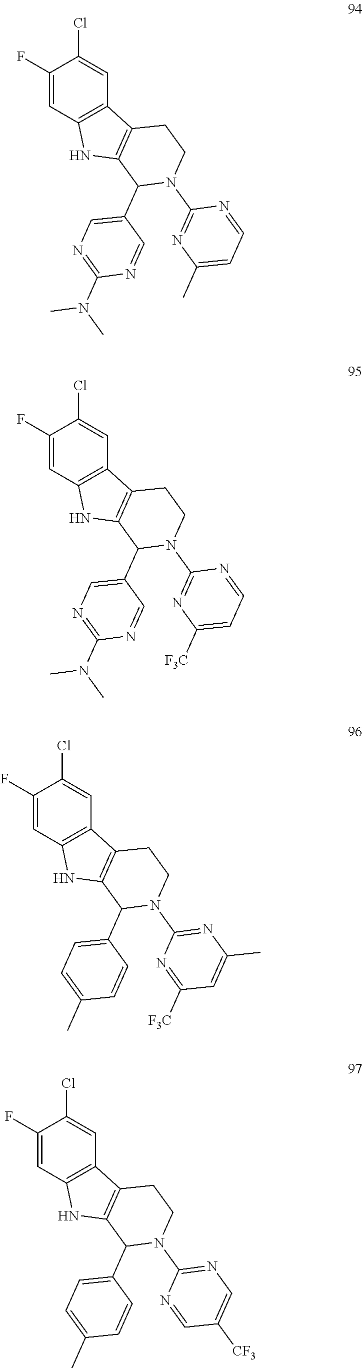 [SMILES: Cc1ccnc(N2CCc3c([nH]c4cc(F)c(Cl)cc34)C2c2cnc(N(C)C)nc2)n1 ; CN(C)c1ncc(C2c3[nH]c4cc(F)c(Cl)cc4c3CCN2c2nccc(C(F)(F)F)n2)cn1 ; Cc1ccc(C2c3[nH]c4cc(F)c(Cl)cc4c3CCN2c2nc(C)cc(C(F)(F)F)n2)cc1 ; Cc1ccc(C2c3[nH]c4cc(F)c(Cl)cc4c3CCN2c2ncc(C(F)(F)F)cn2)cc1]

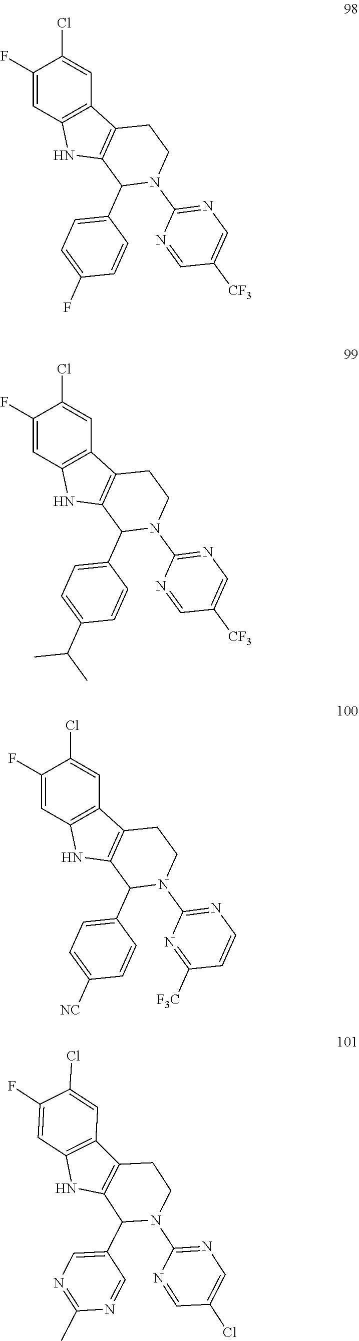

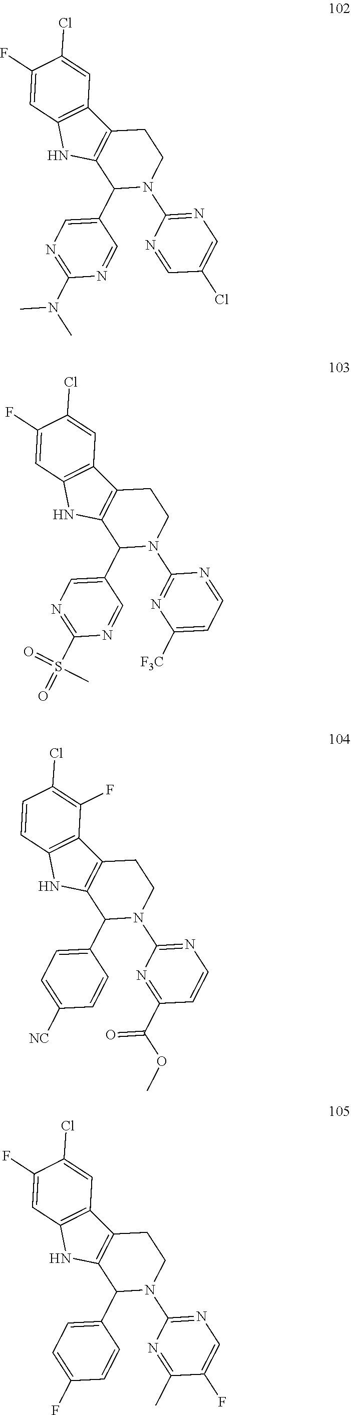

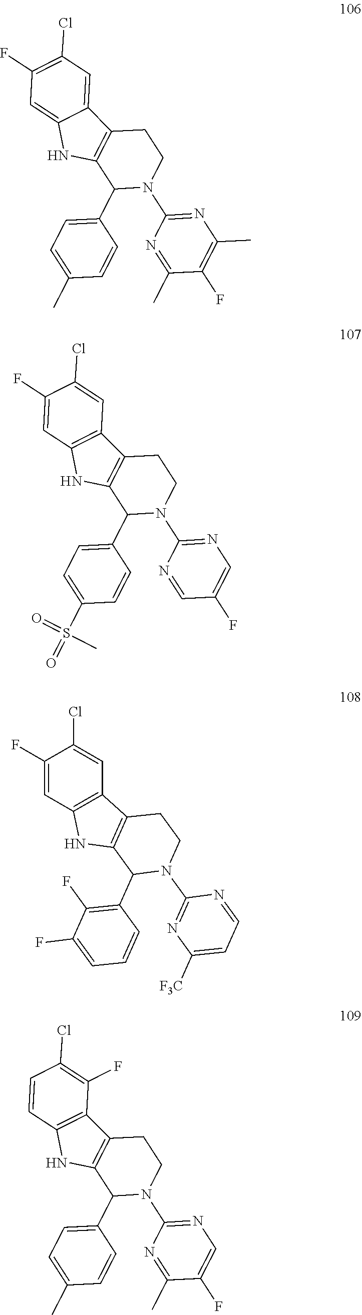 [SMILES: Cc1ccc(C2c3[nH]c4cc(F)c(Cl)cc4c3CCN2c2nc(C)c(F)c(C)n2)cc1 ; CS(=O)(=O)c1ccc(C2c3[nH]c4cc(F)c(Cl)cc4c3CCN2c2ncc(F)cn2)cc1 ; Fc1cc2[nH]c3c(c2cc1Cl)CCN(c1nccc(C(F)(F)F)n1)C3c1cccc(F)c1F ; Cc1ccc(C2c3[nH]c4ccc(Cl)c(F)c4c3CCN2c2ncc(F)c(C)n2)cc1]

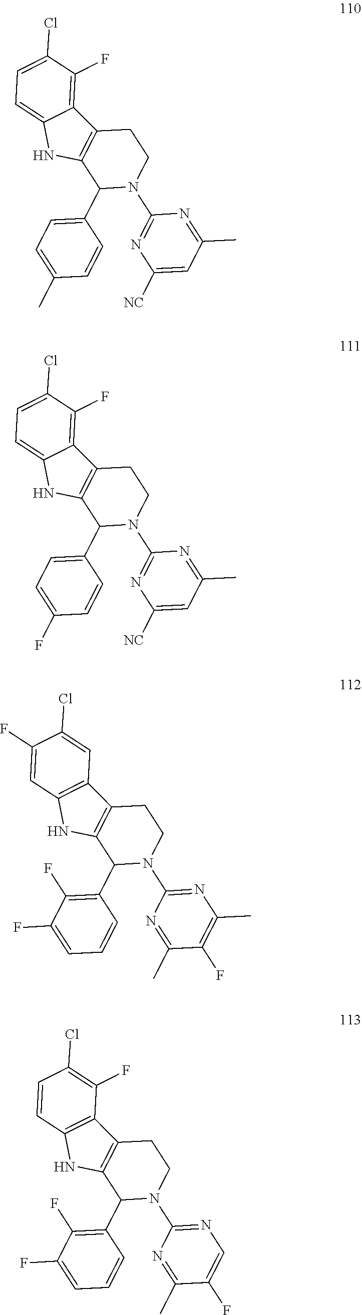 [SMILES: Cc1ccc(C2c3[nH]c4ccc(Cl)c(F)c4c3CCN2c2nc(C)cc(C#N)n2)cc1 ; Cc1cc(C#N)nc(N2CCc3c([nH]c4ccc(Cl)c(F)c34)C2c2ccc(F)cc2)n1 ; Cc1nc(N2CCc3c([nH]c4cc(F)c(Cl)cc34)C2c2cccc(F)c2F)nc(C)c1F ; Cc1nc(N2CCc3c([nH]c4ccc(Cl)c(F)c34)C2c2cccc(F)c2F)ncc1F]

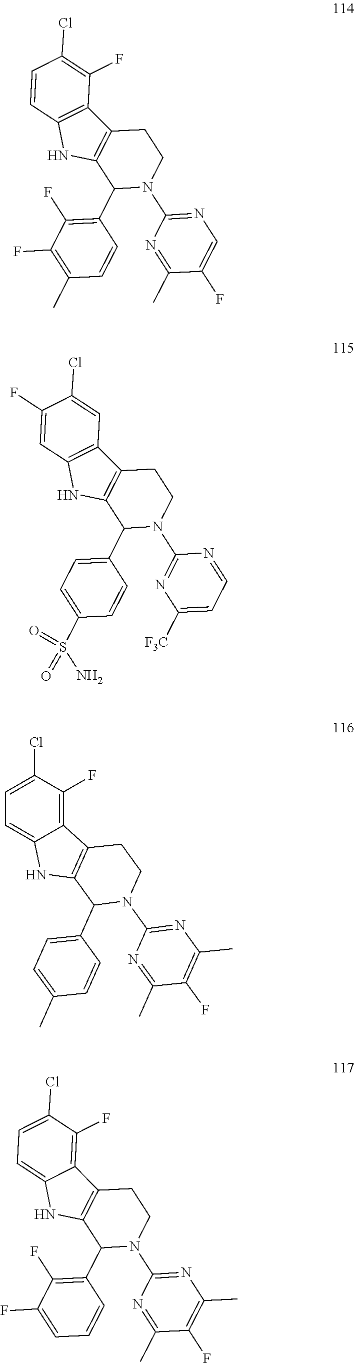 [SMILES: Cc1ccc(C2c3[nH]c4ccc(Cl)c(F)c4c3CCN2c2ncc(F)c(C)n2)c(F)c1F ; NS(=O)(=O)c1ccc(C2c3[nH]c4cc(F)c(Cl)cc4c3CCN2c2nccc(C(F)(F)F)n2)cc1 ; Cc1ccc(C2c3[nH]c4ccc(Cl)c(F)c4c3CCN2c2nc(C)c(F)c(C)n2)cc1 ; Cc1nc(N2CCc3c([nH]c4ccc(Cl)c(F)c34)C2c2cccc(F)c2F)nc(C)c1F]

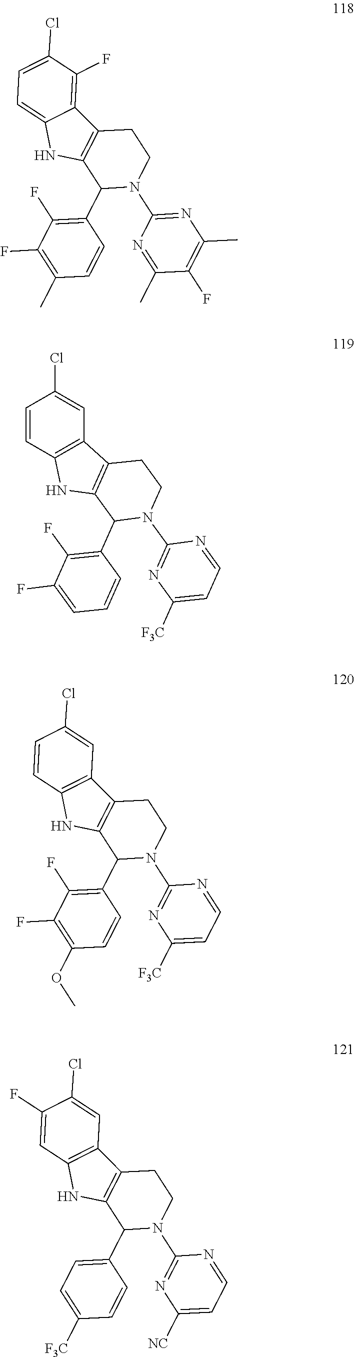 [SMILES: Cc1ccc(C2c3[nH]c4ccc(Cl)c(F)c4c3CCN2c2nc(C)c(F)c(C)n2)c(F)c1F ; Fc1cccc(C2c3[nH]c4ccc(Cl)cc4c3CCN2c2nccc(C(F)(F)F)n2)c1F ; COc1ccc(C2c3[nH]c4ccc(Cl)cc4c3CCN2c2nccc(C(F)(F)F)n2)c(F)c1F ; N#Cc1ccnc(N2CCc3c([nH]c4cc(F)c(Cl)cc34)C2c2ccc(C(F)(F)F)cc2)n1]

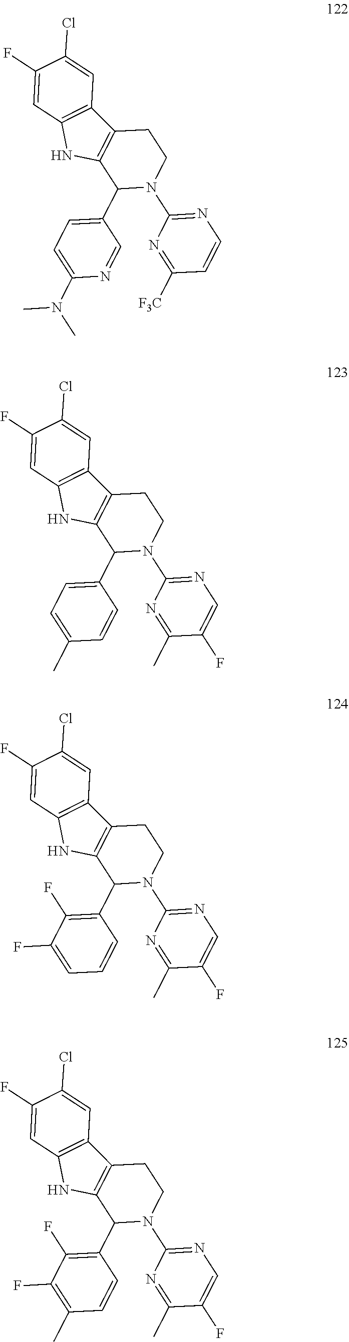 [SMILES: CN(C)c1ccc(C2c3[nH]c4cc(F)c(Cl)cc4c3CCN2c2nccc(C(F)(F)F)n2)cn1 ; Cc1ccc(C2c3[nH]c4cc(F)c(Cl)cc4c3CCN2c2ncc(F)c(C)n2)cc1 ; Cc1nc(N2CCc3c([nH]c4cc(F)c(Cl)cc34)C2c2cccc(F)c2F)ncc1F ; Cc1ccc(C2c3[nH]c4cc(F)c(Cl)cc4c3CCN2c2ncc(F)c(C)n2)c(F)c1F]

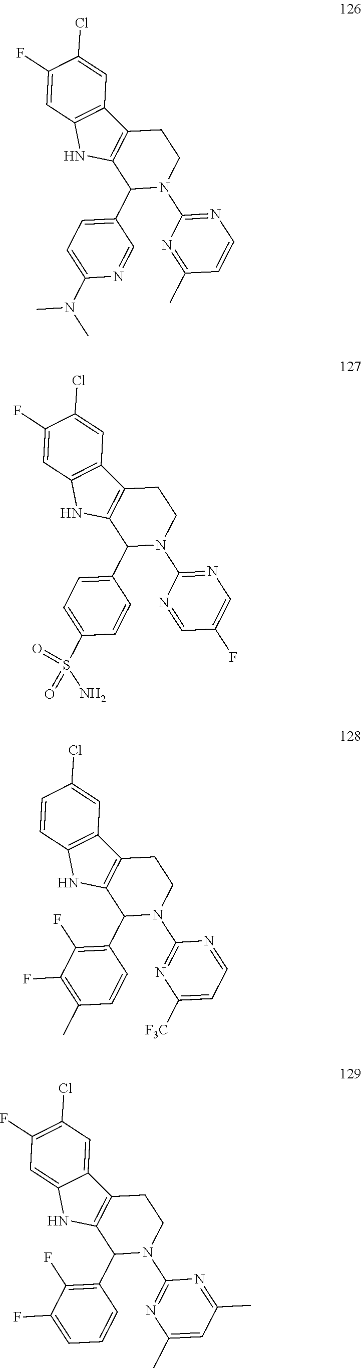

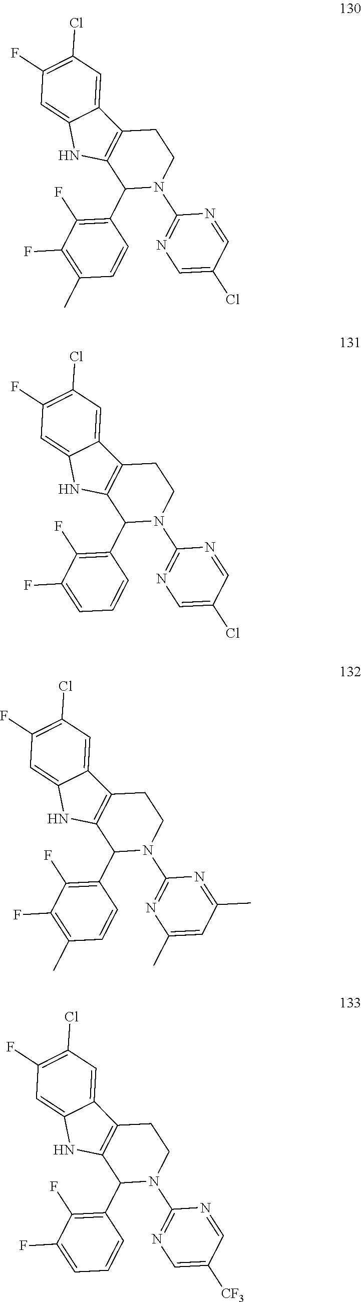 [SMILES: Cc1ccc(C2c3[nH]c4cc(F)c(Cl)cc4c3CCN2c2ncc(Cl)cn2)c(F)c1F ; Fc1cc2[nH]c3c(c2cc1Cl)CCN(c1ncc(Cl)cn1)C3c1cccc(F)c1F ; Cc1cc(C)nc(N2CCc3c([nH]c4cc(F)c(Cl)cc34)C2c2ccc(C)c(F)c2F)n1 ; Fc1cc2[nH]c3c(c2cc1Cl)CCN(c1ncc(C(F)(F)F)cn1)C3c1cccc(F)c1F]

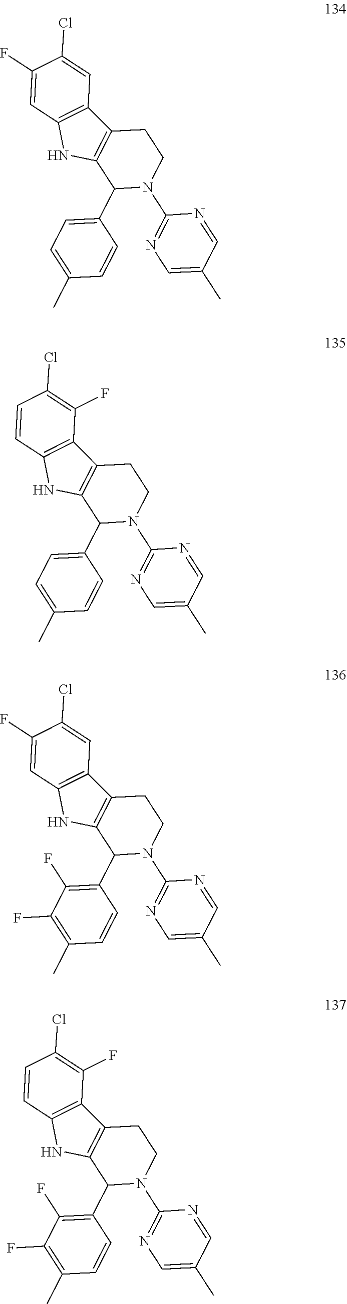

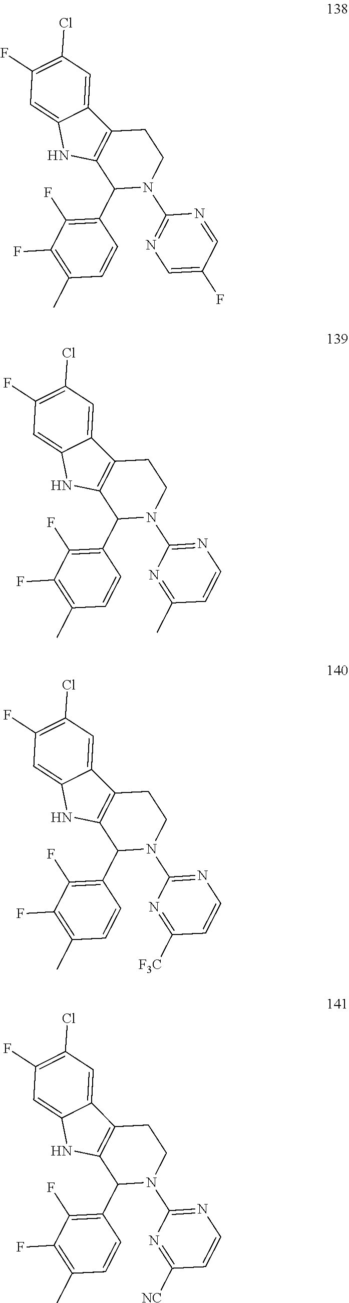 [SMILES: Cc1ccc(C2c3[nH]c4cc(F)c(Cl)cc4c3CCN2c2ncc(F)cn2)c(F)c1F ; Cc1ccnc(N2CCc3c([nH]c4cc(F)c(Cl)cc34)C2c2ccc(C)c(F)c2F)n1 ; Cc1ccc(C2c3[nH]c4cc(F)c(Cl)cc4c3CCN2c2nccc(C(F)(F)F)n2)c(F)c1F ; Cc1ccc(C2c3[nH]c4cc(F)c(Cl)cc4c3CCN2c2nccc(C#N)n2)c(F)c1F]

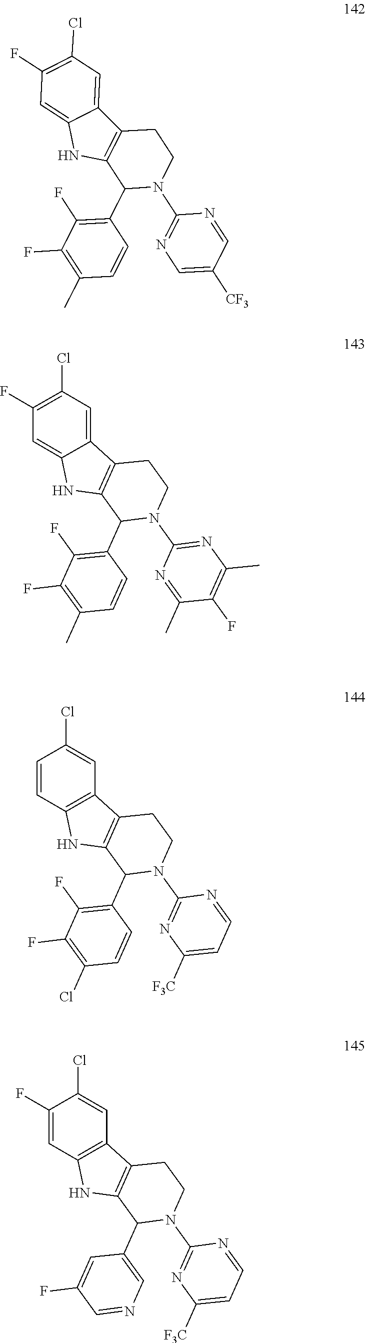 [SMILES: Cc1ccc(C2c3[nH]c4cc(F)c(Cl)cc4c3CCN2c2ncc(C(F)(F)F)cn2)c(F)c1F ; Cc1ccc(C2c3[nH]c4cc(F)c(Cl)cc4c3CCN2c2nc(C)c(F)c(C)n2)c(F)c1F ; Fc1c(Cl)ccc(C2c3[nH]c4ccc(Cl)cc4c3CCN2c2nccc(C(F)(F)F)n2)c1F ; Fc1cncc(C2c3[nH]c4cc(F)c(Cl)cc4c3CCN2c2nccc(C(F)(F)F)n2)c1]

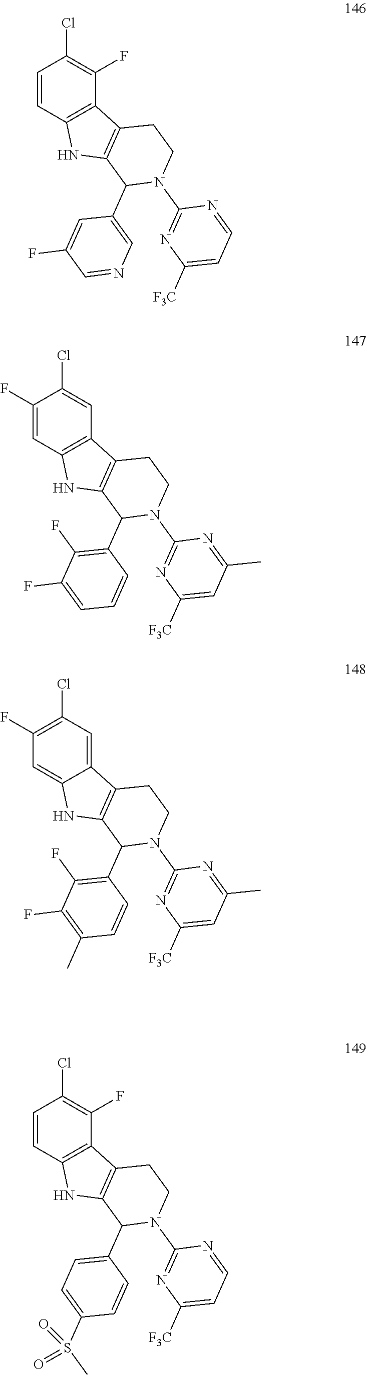 [SMILES: Fc1cncc(C2c3[nH]c4ccc(Cl)c(F)c4c3CCN2c2nccc(C(F)(F)F)n2)c1 ; Cc1cc(C(F)(F)F)nc(N2CCc3c([nH]c4cc(F)c(Cl)cc34)C2c2cccc(F)c2F)n1 ; Cc1cc(C(F)(F)F)nc(N2CCc3c([nH]c4cc(F)c(Cl)cc34)C2c2ccc(C)c(F)c2F)n1 ; CS(=O)(=O)c1ccc(C2c3[nH]c4ccc(Cl)c(F)c4c3CCN2c2nccc(C(F)(F)F)n2)cc1]

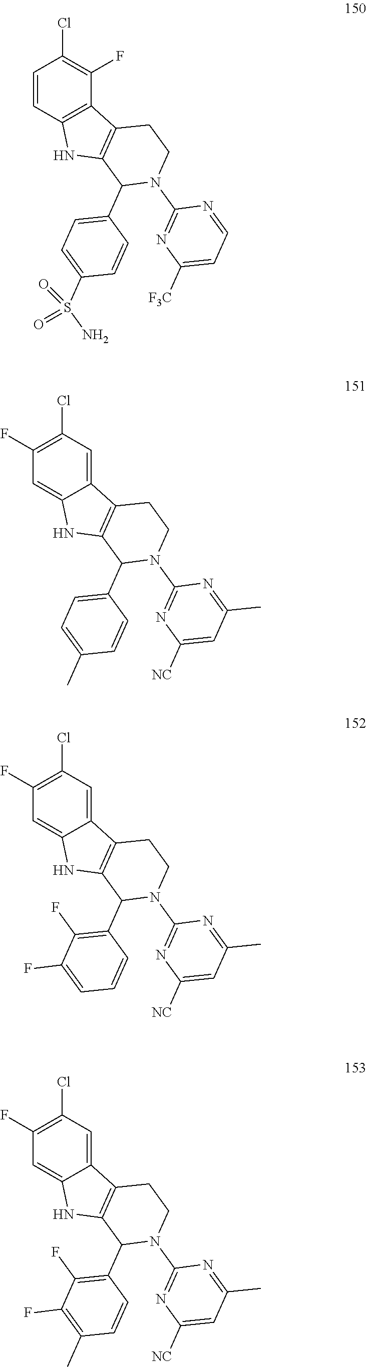 [SMILES: NS(=O)(=O)c1ccc(C2c3[nH]c4ccc(Cl)c(F)c4c3CCN2c2nccc(C(F)(F)F)n2)cc1 ; Cc1ccc(C2c3[nH]c4cc(F)c(Cl)cc4c3CCN2c2nc(C)cc(C#N)n2)cc1 ; Cc1cc(C#N)nc(N2CCc3c([nH]c4cc(F)c(Cl)cc34)C2c2cccc(F)c2F)n1 ; Cc1cc(C#N)nc(N2CCc3c([nH]c4cc(F)c(Cl)cc34)C2c2ccc(C)c(F)c2F)n1]

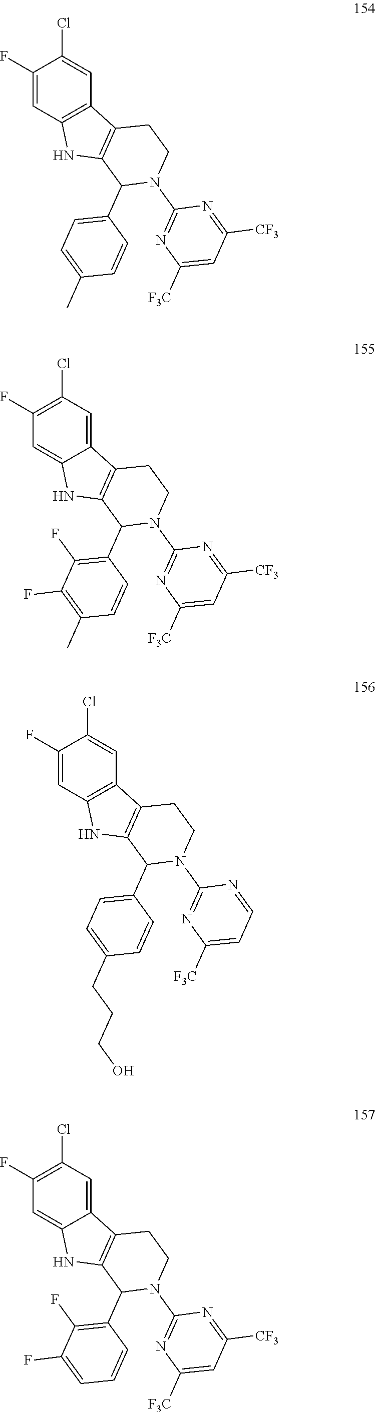 [SMILES: Cc1ccc(C2c3[nH]c4cc(F)c(Cl)cc4c3CCN2c2nc(C(F)(F)F)cc(C(F)(F)F)n2)cc1 ; Cc1ccc(C2c3[nH]c4cc(F)c(Cl)cc4c3CCN2c2nc(C(F)(F)F)cc(C(F)(F)F)n2)c(F)c1F ; OCCCc1ccc(C2c3[nH]c4cc(F)c(Cl)cc4c3CCN2c2nccc(C(F)(F)F)n2)cc1 ; Fc1cc2[nH]c3c(c2cc1Cl)CCN(c1nc(C(F)(F)F)cc(C(F)(F)F)n1)C3c1cccc(F)c1F]

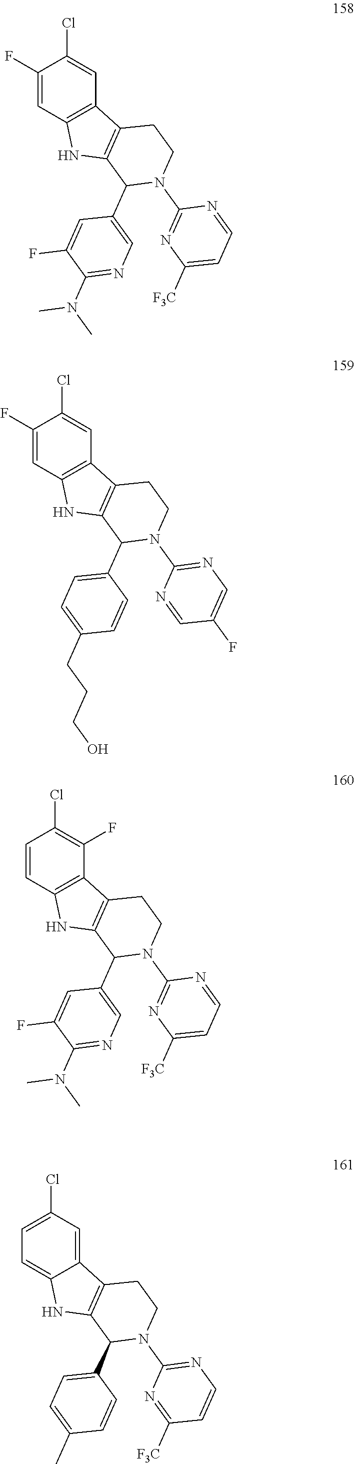 [SMILES: CN(C)c1ncc(C2c3[nH]c4cc(F)c(Cl)cc4c3CCN2c2nccc(C(F)(F)F)n2)cc1F ; OCCCc1ccc(C2c3[nH]c4cc(F)c(Cl)cc4c3CCN2c2ncc(F)cn2)cc1 ; CN(C)c1ncc(C2c3[nH]c4ccc(Cl)c(F)c4c3CCN2c2nccc(C(F)(F)F)n2)cc1F ; Cc1ccc([C@H]2c3[nH]c4ccc(Cl)cc4c3CCN2c2nccc(C(F)(F)F)n2)cc1]

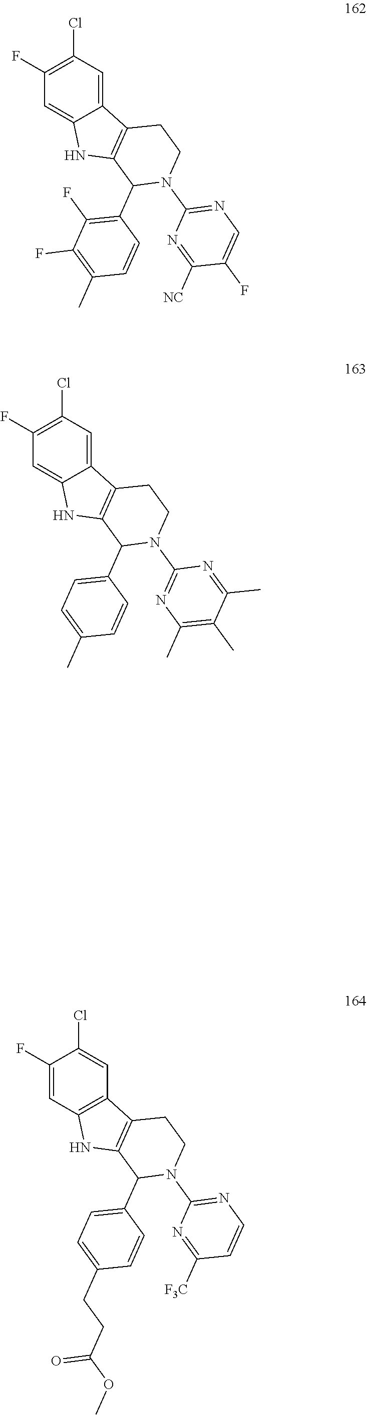 [SMILES: Cc1ccc(C2c3[nH]c4cc(F)c(Cl)cc4c3CCN2c2ncc(F)c(C#N)n2)c(F)c1F ; Cc1ccc(C2c3[nH]c4cc(F)c(Cl)cc4c3CCN2c2nc(C)c(C)c(C)n2)cc1 ; COC(=O)CCc1ccc(C2c3[nH]c4cc(F)c(Cl)cc4c3CCN2c2nccc(C(F)(F)F)n2)cc1]

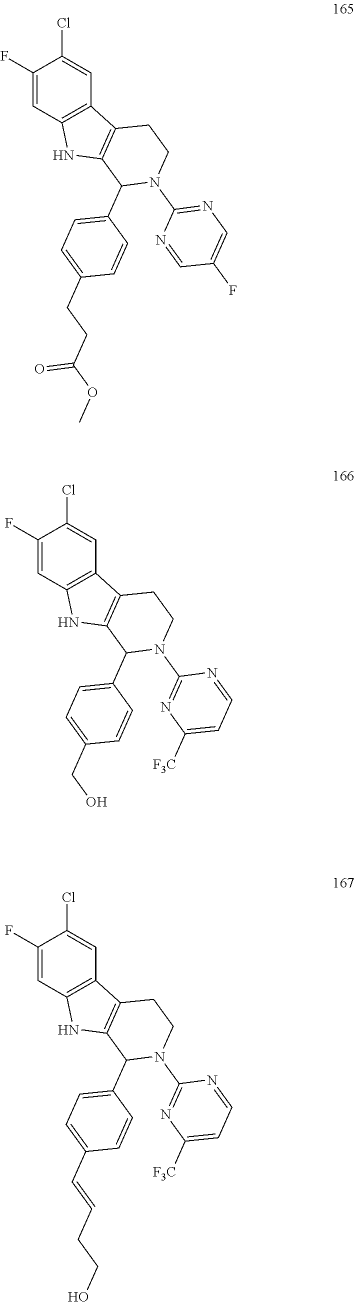 [SMILES: COC(=O)CCc1ccc(C2c3[nH]c4cc(F)c(Cl)cc4c3CCN2c2ncc(F)cn2)cc1 ; OCc1ccc(C2c3[nH]c4cc(F)c(Cl)cc4c3CCN2c2nccc(C(F)(F)F)n2)cc1 ; OCC/C=C/c1ccc(C2c3[nH]c4cc(F)c(Cl)cc4c3CCN2c2nccc(C(F)(F)F)n2)cc1]

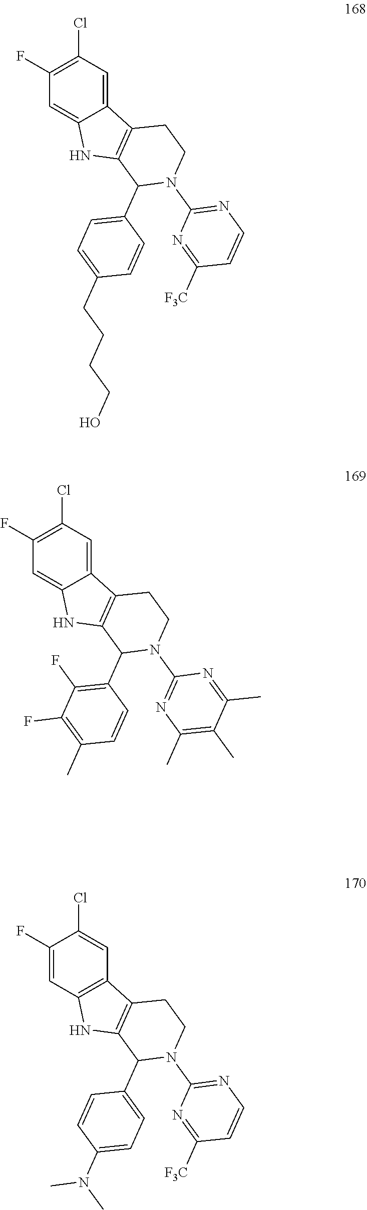 [SMILES: OCCCCc1ccc(C2c3[nH]c4cc(F)c(Cl)cc4c3CCN2c2nccc(C(F)(F)F)n2)cc1 ; Cc1ccc(C2c3[nH]c4cc(F)c(Cl)cc4c3CCN2c2nc(C)c(C)c(C)n2)c(F)c1F ; CN(C)c1ccc(C2c3[nH]c4cc(F)c(Cl)cc4c3CCN2c2nccc(C(F)(F)F)n2)cc1]

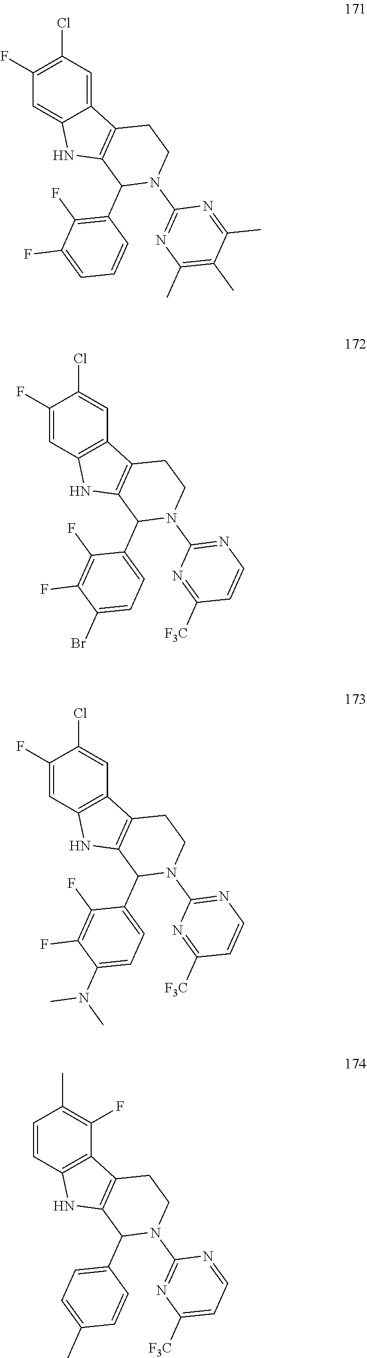 [SMILES: Cc1nc(N2CCc3c([nH]c4cc(F)c(Cl)cc34)C2c2cccc(F)c2F)nc(C)c1C ; Fc1cc2[nH]c3c(c2cc1Cl)CCN(c1nccc(C(F)(F)F)n1)C3c1ccc(Br)c(F)c1F ; CN(C)c1ccc(C2c3[nH]c4cc(F)c(Cl)cc4c3CCN2c2nccc(C(F)(F)F)n2)c(F)c1F ; Cc1ccc(C2c3[nH]c4ccc(C)c(F)c4c3CCN2c2nccc(C(F)(F)F)n2)cc1]

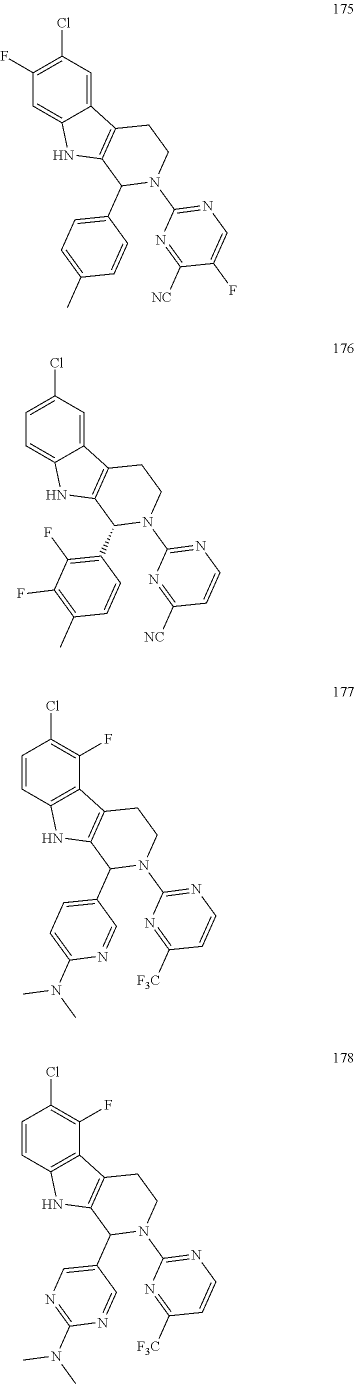 [SMILES: Cc1ccc(C2c3[nH]c4cc(F)c(Cl)cc4c3CCN2c2ncc(F)c(C#N)n2)cc1 ; Cc1ccc([C@@H]2c3[nH]c4ccc(Cl)cc4c3CCN2c2nccc(C#N)n2)c(F)c1F ; CN(C)c1ccc(C2c3[nH]c4ccc(Cl)c(F)c4c3CCN2c2nccc(C(F)(F)F)n2)cn1 ; CN(C)c1ncc(C2c3[nH]c4ccc(Cl)c(F)c4c3CCN2c2nccc(C(F)(F)F)n2)cn1]

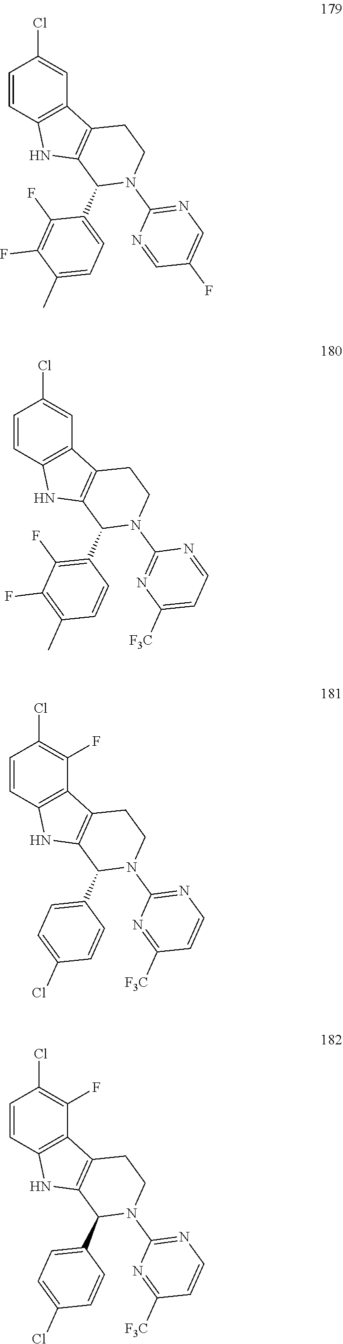 [SMILES: Cc1ccc([C@@H]2c3[nH]c4ccc(Cl)cc4c3CCN2c2ncc(F)cn2)c(F)c1F ; Cc1ccc([C@@H]2c3[nH]c4ccc(Cl)cc4c3CCN2c2nccc(C(F)(F)F)n2)c(F)c1F ; Fc1c(Cl)ccc2[nH]c3c(c12)CCN(c1nccc(C(F)(F)F)n1)[C@@H]3c1ccc(Cl)cc1 ; Fc1c(Cl)ccc2[nH]c3c(c12)CCN(c1nccc(C(F)(F)F)n1)[C@H]3c1ccc(Cl)cc1]

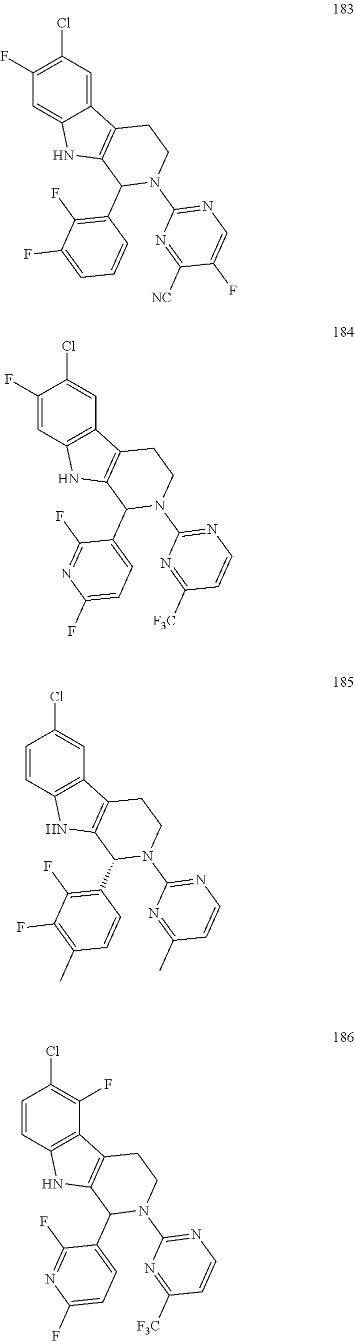 [SMILES: N#Cc1nc(N2CCc3c([nH]c4cc(F)c(Cl)cc34)C2c2cccc(F)c2F)ncc1F ; Fc1ccc(C2c3[nH]c4cc(F)c(Cl)cc4c3CCN2c2nccc(C(F)(F)F)n2)c(F)n1 ; Cc1ccnc(N2CCc3c([nH]c4ccc(Cl)cc34)[C@H]2c2ccc(C)c(F)c2F)n1 ; Fc1ccc(C2c3[nH]c4ccc(Cl)c(F)c4c3CCN2c2nccc(C(F)(F)F)n2)c(F)n1]

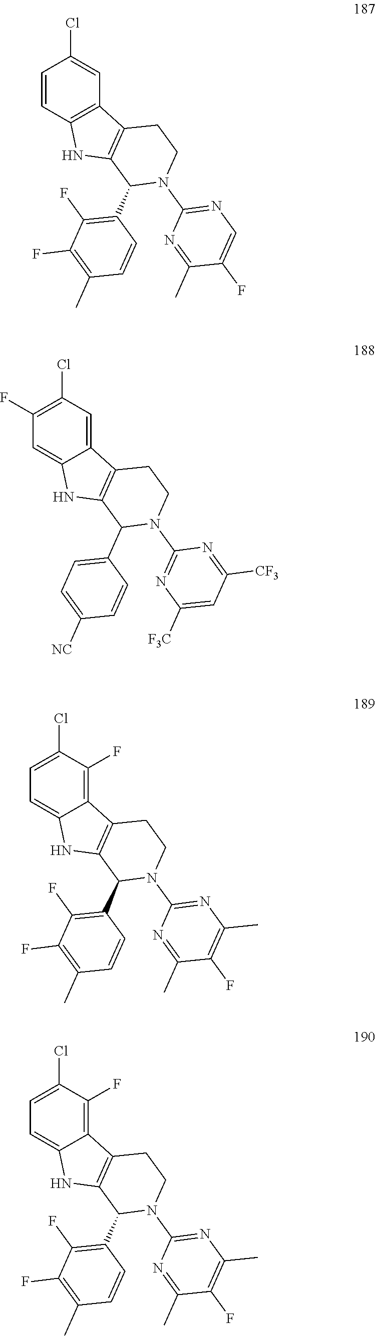 [SMILES: Cc1ccc([C@@H]2c3[nH]c4ccc(Cl)cc4c3CCN2c2ncc(F)c(C)n2)c(F)c1F ; N#Cc1ccc(C2c3[nH]c4cc(F)c(Cl)cc4c3CCN2c2nc(C(F)(F)F)cc(C(F)(F)F)n2)cc1 ; Cc1ccc([C@H]2c3[nH]c4ccc(Cl)c(F)c4c3CCN2c2nc(C)c(F)c(C)n2)c(F)c1F ; Cc1ccc([C@@H]2c3[nH]c4ccc(Cl)c(F)c4c3CCN2c2nc(C)c(F)c(C)n2)c(F)c1F]

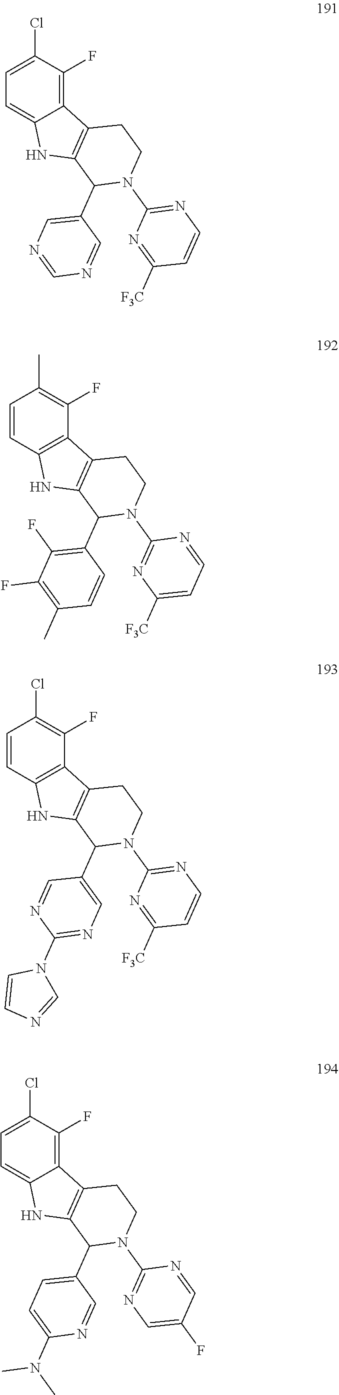

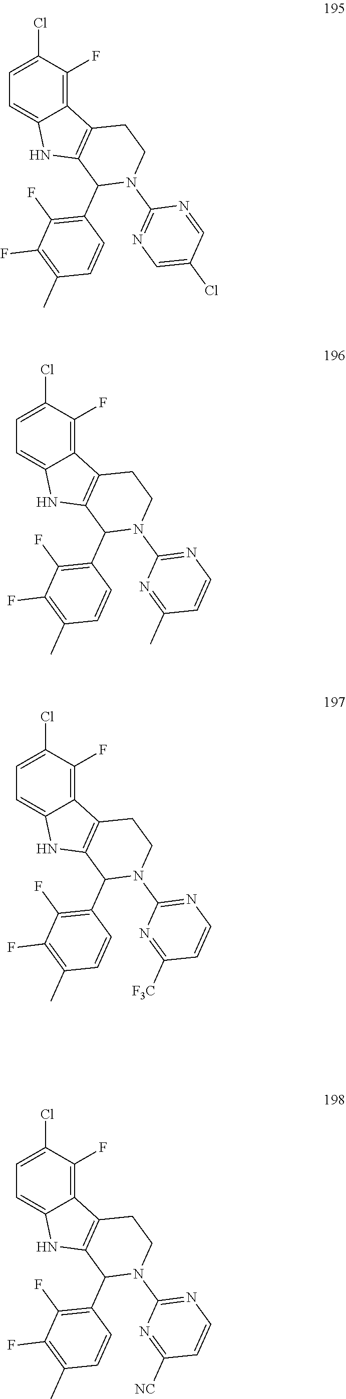 [SMILES: Cc1ccc(C2c3[nH]c4ccc(Cl)c(F)c4c3CCN2c2ncc(Cl)cn2)c(F)c1F ; Cc1ccnc(N2CCc3c([nH]c4ccc(Cl)c(F)c34)C2c2ccc(C)c(F)c2F)n1 ; Cc1ccc(C2c3[nH]c4ccc(Cl)c(F)c4c3CCN2c2nccc(C(F)(F)F)n2)c(F)c1F ; Cc1ccc(C2c3[nH]c4ccc(Cl)c(F)c4c3CCN2c2nccc(C#N)n2)c(F)c1F]

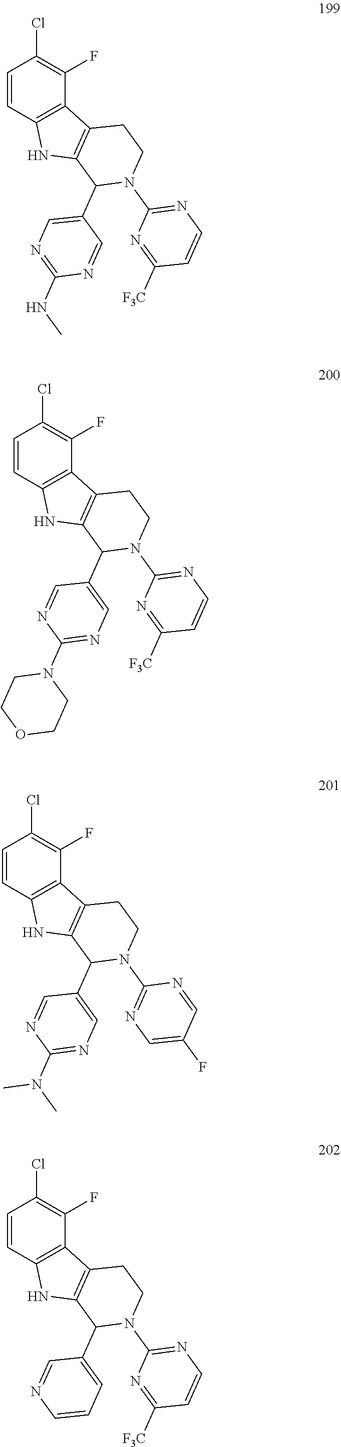 [SMILES: CNc1ncc(C2c3[nH]c4ccc(Cl)c(F)c4c3CCN2c2nccc(C(F)(F)F)n2)cn1 ; Fc1c(Cl)ccc2[nH]c3c(c12)CCN(c1nccc(C(F)(F)F)n1)C3c1cnc(N2CCOCC2)nc1 ; CN(C)c1ncc(C2c3[nH]c4ccc(Cl)c(F)c4c3CCN2c2ncc(F)cn2)cn1 ; Fc1c(Cl)ccc2[nH]c3c(c12)CCN(c1nccc(C(F)(F)F)n1)C3c1cccnc1]

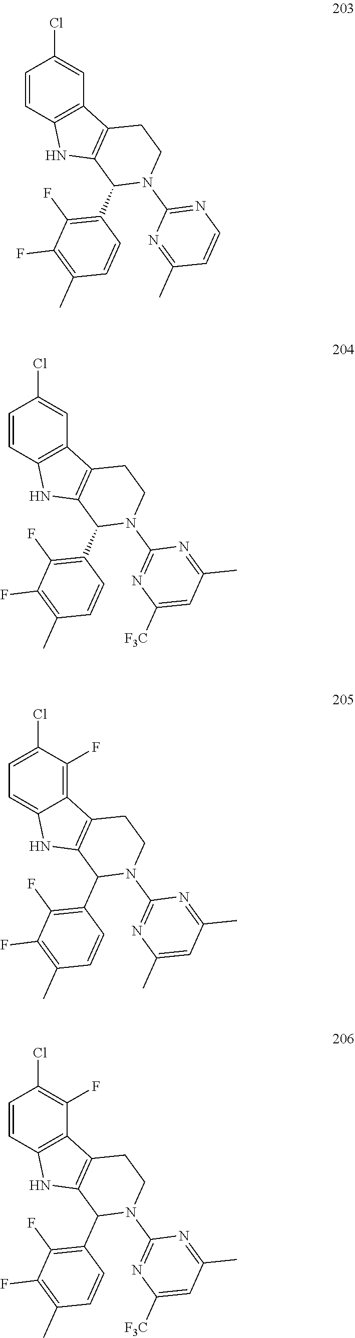 [SMILES: Cc1ccnc(N2CCc3c([nH]c4ccc(Cl)cc34)[C@H]2c2ccc(C)c(F)c2F)n1 ; Cc1cc(C(F)(F)F)nc(N2CCc3c([nH]c4ccc(Cl)cc34)[C@H]2c2ccc(C)c(F)c2F)n1 ; Cc1cc(C)nc(N2CCc3c([nH]c4ccc(Cl)c(F)c34)C2c2ccc(C)c(F)c2F)n1 ; Cc1cc(C(F)(F)F)nc(N2CCc3c([nH]c4ccc(Cl)c(F)c34)C2c2ccc(C)c(F)c2F)n1]

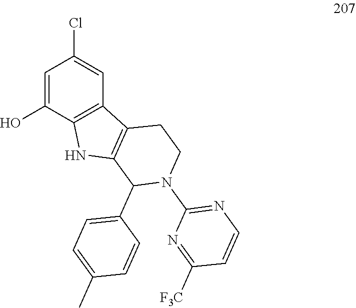 [SMILES: Cc1ccc(C2c3[nH]c4c(O)cc(Cl)cc4c3CCN2c2nccc(C(F)(F)F)n2)cc1]

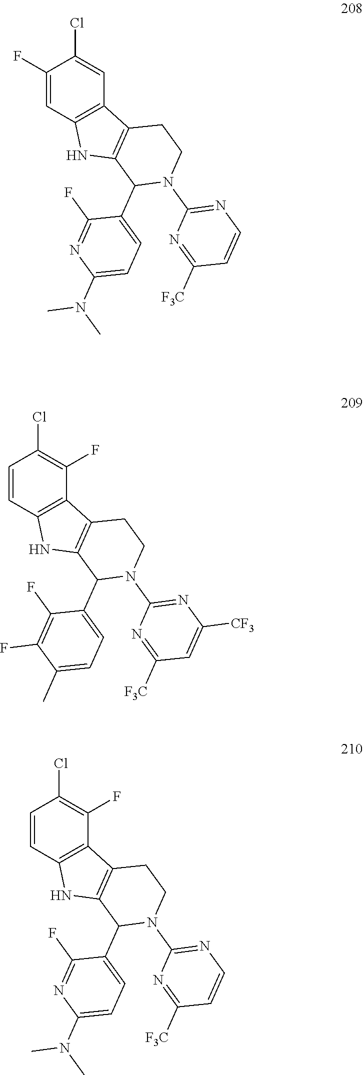 [SMILES: CN(C)c1ccc(C2c3[nH]c4cc(F)c(Cl)cc4c3CCN2c2nccc(C(F)(F)F)n2)c(F)n1 ; Cc1ccc(C2c3[nH]c4ccc(Cl)c(F)c4c3CCN2c2nc(C(F)(F)F)cc(C(F)(F)F)n2)c(F)c1F ; CN(C)c1ccc(C2c3[nH]c4ccc(Cl)c(F)c4c3CCN2c2nccc(C(F)(F)F)n2)c(F)n1]

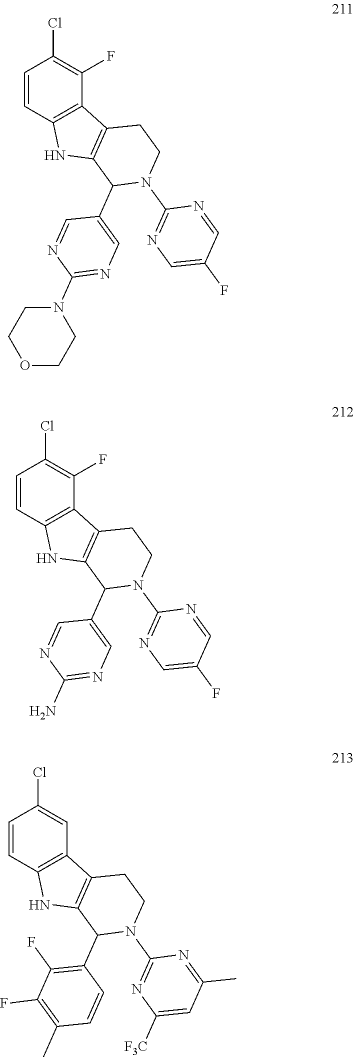 [SMILES: Fc1cnc(N2CCc3c([nH]c4ccc(Cl)c(F)c34)C2c2cnc(N3CCOCC3)nc2)nc1 ; Nc1ncc(C2c3[nH]c4ccc(Cl)c(F)c4c3CCN2c2ncc(F)cn2)cn1 ; Cc1cc(C(F)(F)F)nc(N2CCc3c([nH]c4ccc(Cl)cc34)C2c2ccc(C)c(F)c2F)n1]

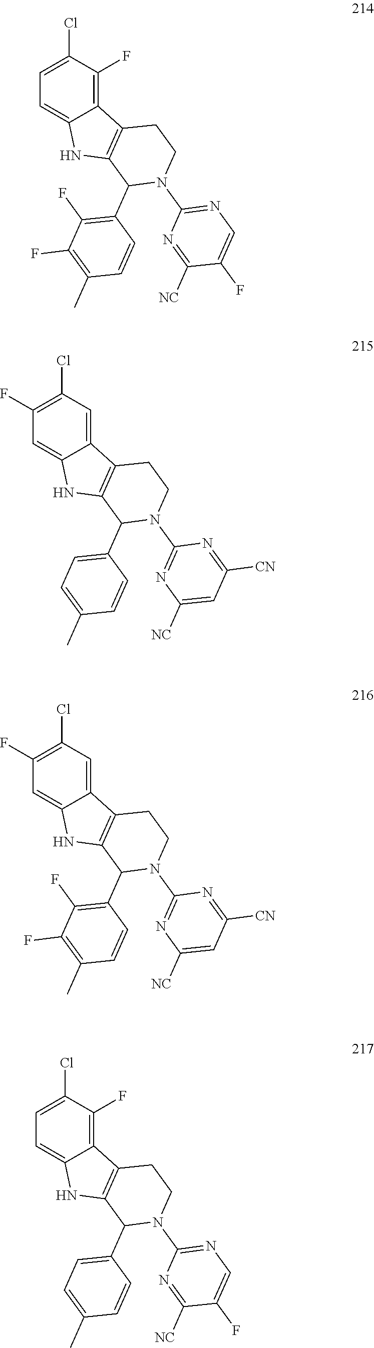 [SMILES: Cc1ccc(C2c3[nH]c4ccc(Cl)c(F)c4c3CCN2c2ncc(F)c(C#N)n2)c(F)c1F ; Cc1ccc(C2c3[nH]c4cc(F)c(Cl)cc4c3CCN2c2nc(C#N)cc(C#N)n2)cc1 ; Cc1ccc(C2c3[nH]c4cc(F)c(Cl)cc4c3CCN2c2nc(C#N)cc(C#N)n2)c(F)c1F ; Cc1ccc(C2c3[nH]c4ccc(Cl)c(F)c4c3CCN2c2ncc(F)c(C#N)n2)cc1]

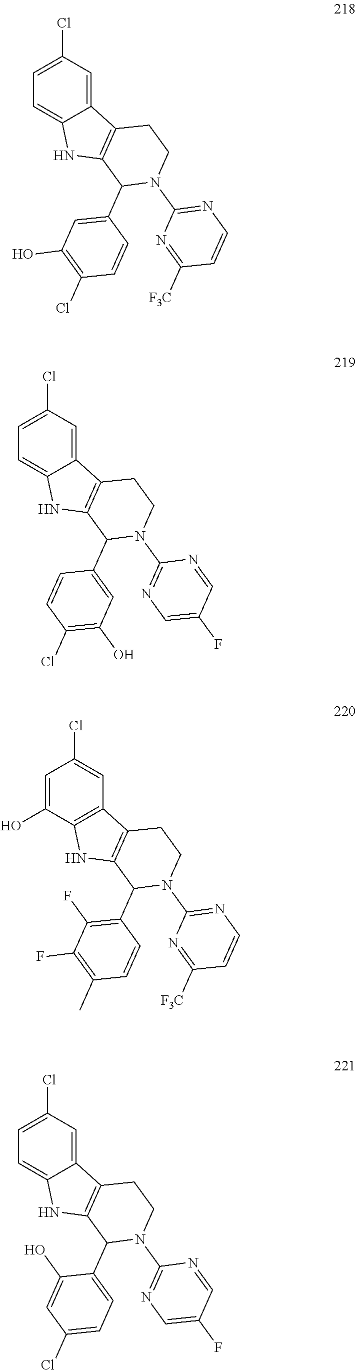 [SMILES: Oc1cc(C2c3[nH]c4ccc(Cl)cc4c3CCN2c2nccc(C(F)(F)F)n2)ccc1Cl ; Oc1cc(C2c3[nH]c4ccc(Cl)cc4c3CCN2c2ncc(F)cn2)ccc1Cl ; Cc1ccc(C2c3[nH]c4c(O)cc(Cl)cc4c3CCN2c2nccc(C(F)(F)F)n2)c(F)c1F ; Oc1cc(Cl)ccc1C1c2[nH]c3ccc(Cl)cc3c2CCN1c1ncc(F)cn1]

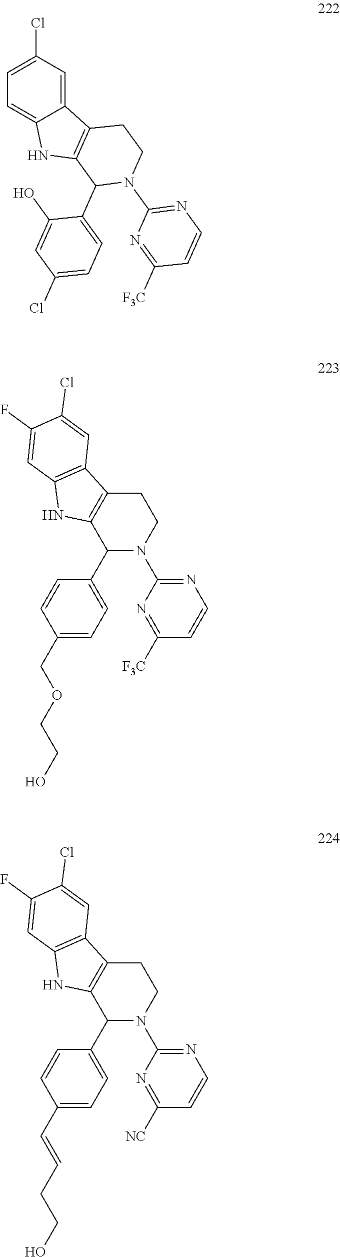 [SMILES: Oc1cc(Cl)ccc1C1c2[nH]c3ccc(Cl)cc3c2CCN1c1nccc(C(F)(F)F)n1 ; OCCOCc1ccc(C2c3[nH]c4cc(F)c(Cl)cc4c3CCN2c2nccc(C(F)(F)F)n2)cc1 ; N#Cc1ccnc(N2CCc3c([nH]c4cc(F)c(Cl)cc34)C2c2ccc(/C=C/CCO)cc2)n1]

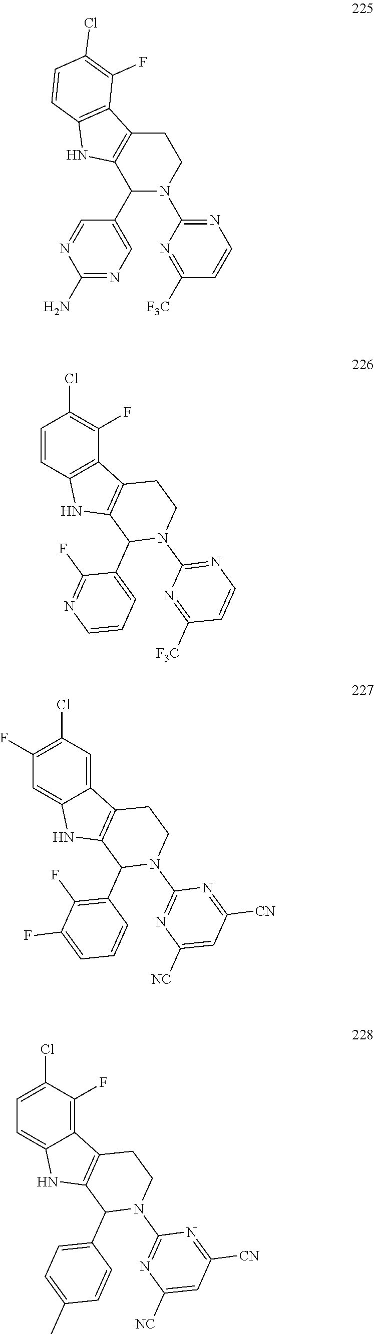 [SMILES: Nc1ncc(C2c3[nH]c4ccc(Cl)c(F)c4c3CCN2c2nccc(C(F)(F)F)n2)cn1 ; Fc1ncccc1C1c2[nH]c3ccc(Cl)c(F)c3c2CCN1c1nccc(C(F)(F)F)n1 ; N#Cc1cc(C#N)nc(N2CCc3c([nH]c4cc(F)c(Cl)cc34)C2c2cccc(F)c2F)n1 ; Cc1ccc(C2c3[nH]c4ccc(Cl)c(F)c4c3CCN2c2nc(C#N)cc(C#N)n2)cc1]

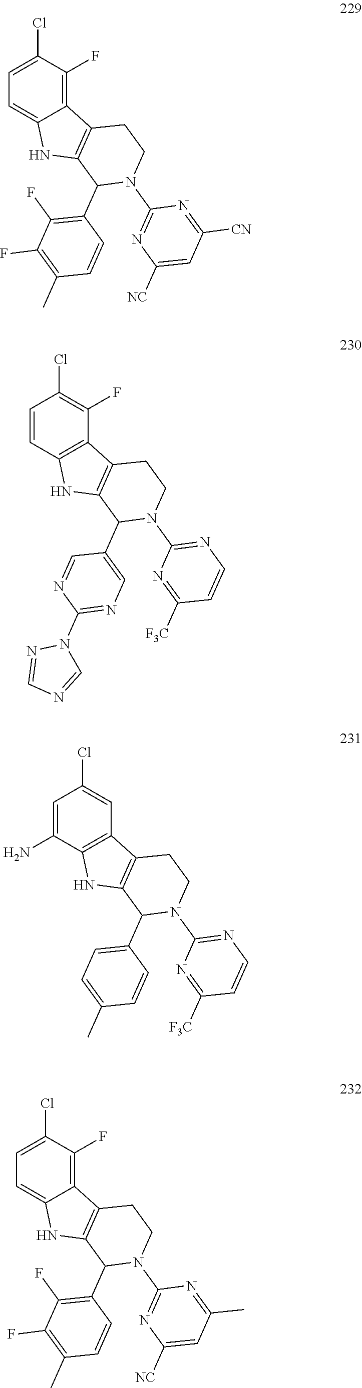 [SMILES: Cc1ccc(C2c3[nH]c4ccc(Cl)c(F)c4c3CCN2c2nc(C#N)cc(C#N)n2)c(F)c1F ; Fc1c(Cl)ccc2[nH]c3c(c12)CCN(c1nccc(C(F)(F)F)n1)C3c1cnc(-n2cncn2)nc1 ; Cc1ccc(C2c3[nH]c4c(N)cc(Cl)cc4c3CCN2c2nccc(C(F)(F)F)n2)cc1 ; Cc1cc(C#N)nc(N2CCc3c([nH]c4ccc(Cl)c(F)c34)C2c2ccc(C)c(F)c2F)n1]

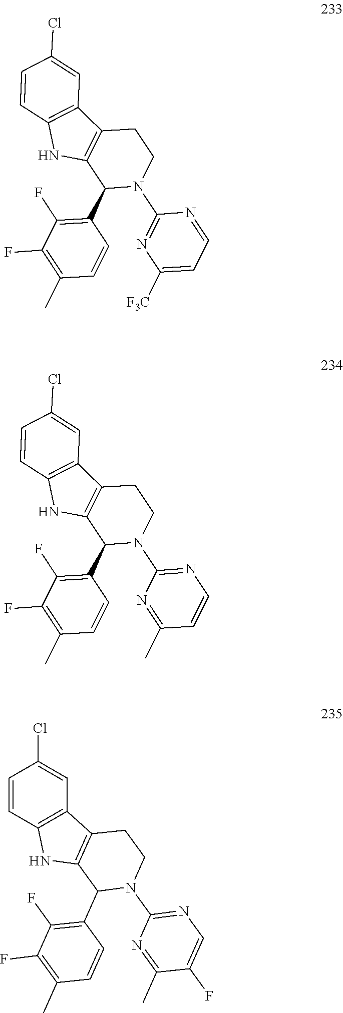 [SMILES: Cc1ccc([C@H]2c3[nH]c4ccc(Cl)cc4c3CCN2c2nccc(C(F)(F)F)n2)c(F)c1F ; Cc1ccnc(N2CCc3c([nH]c4ccc(Cl)cc34)[C@@H]2c2ccc(C)c(F)c2F)n1 ; Cc1ccc(C2c3[nH]c4ccc(Cl)cc4c3CCN2c2ncc(F)c(C)n2)c(F)c1F]

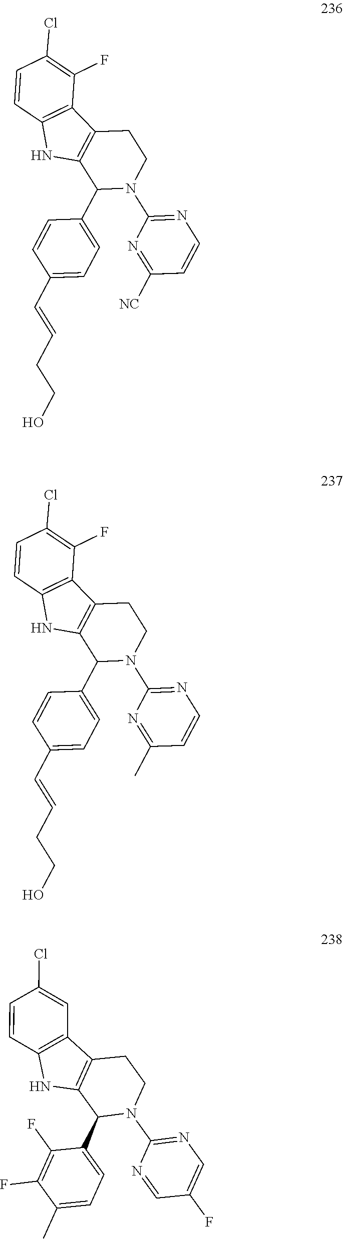 [SMILES: N#Cc1ccnc(N2CCc3c([nH]c4ccc(Cl)c(F)c34)C2c2ccc(/C=C/CCO)cc2)n1 ; Cc1ccnc(N2CCc3c([nH]c4ccc(Cl)c(F)c34)C2c2ccc(/C=C/CCO)cc2)n1 ; Cc1ccc([C@H]2c3[nH]c4ccc(Cl)cc4c3CCN2c2ncc(F)cn2)c(F)c1F]

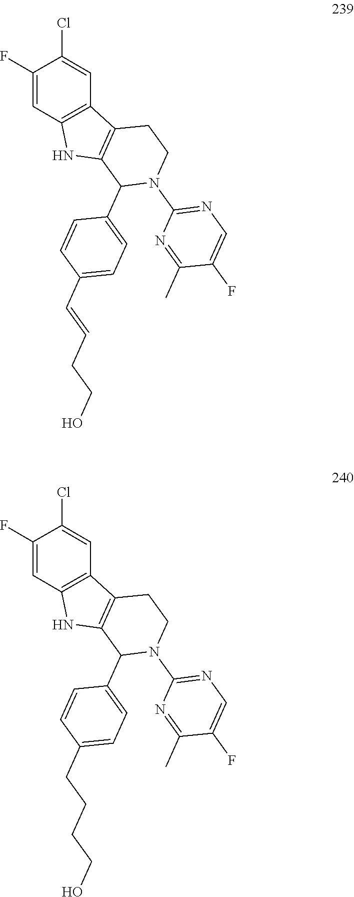 [SMILES: Cc1nc(N2CCc3c([nH]c4cc(F)c(Cl)cc34)C2c2ccc(/C=C/CCO)cc2)ncc1F ; Cc1nc(N2CCc3c([nH]c4cc(F)c(Cl)cc34)C2c2ccc(CCCCO)cc2)ncc1F]

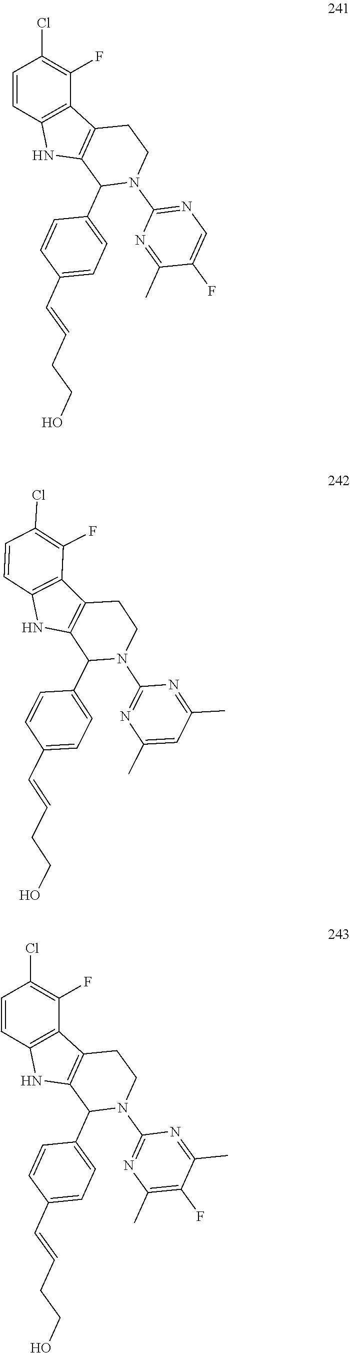 [SMILES: Cc1nc(N2CCc3c([nH]c4ccc(Cl)c(F)c34)C2c2ccc(/C=C/CCO)cc2)ncc1F ; Cc1cc(C)nc(N2CCc3c([nH]c4ccc(Cl)c(F)c34)C2c2ccc(/C=C/CCO)cc2)n1 ; Cc1nc(N2CCc3c([nH]c4ccc(Cl)c(F)c34)C2c2ccc(/C=C/CCO)cc2)nc(C)c1F]

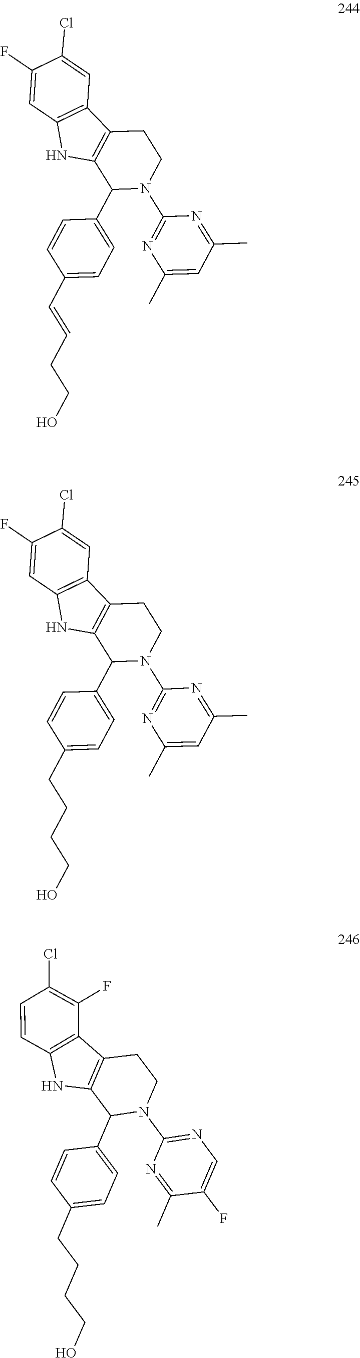 [SMILES: Cc1cc(C)nc(N2CCc3c([nH]c4cc(F)c(Cl)cc34)C2c2ccc(/C=C/CCO)cc2)n1 ; Cc1cc(C)nc(N2CCc3c([nH]c4cc(F)c(Cl)cc34)C2c2ccc(CCCCO)cc2)n1 ; Cc1nc(N2CCc3c([nH]c4ccc(Cl)c(F)c34)C2c2ccc(CCCCO)cc2)ncc1F]

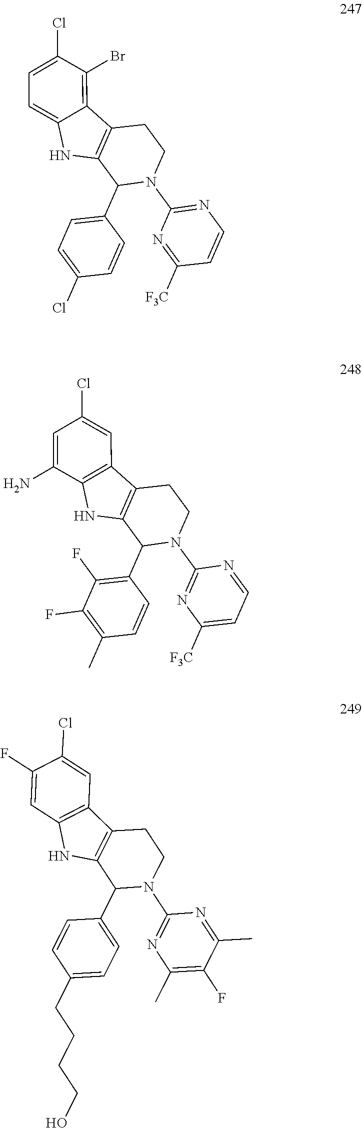 [SMILES: FC(F)(F)c1ccnc(N2CCc3c([nH]c4ccc(Cl)c(Br)c34)C2c2ccc(Cl)cc2)n1 ; Cc1ccc(C2c3[nH]c4c(N)cc(Cl)cc4c3CCN2c2nccc(C(F)(F)F)n2)c(F)c1F ; Cc1nc(N2CCc3c([nH]c4cc(F)c(Cl)cc34)C2c2ccc(CCCCO)cc2)nc(C)c1F]

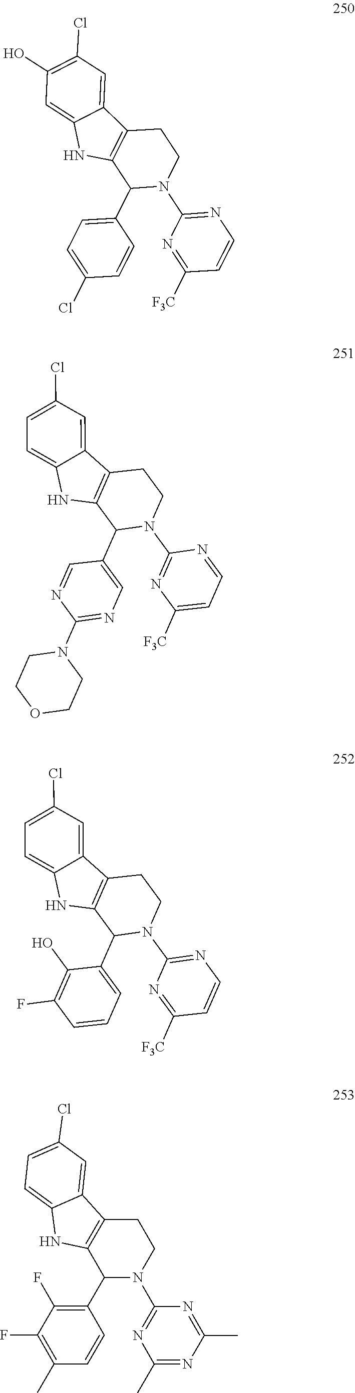 [SMILES: Oc1cc2[nH]c3c(c2cc1Cl)CCN(c1nccc(C(F)(F)F)n1)C3c1ccc(Cl)cc1 ; FC(F)(F)c1ccnc(N2CCc3c([nH]c4ccc(Cl)cc34)C2c2cnc(N3CCOCC3)nc2)n1 ; Oc1c(F)cccc1C1c2[nH]c3ccc(Cl)cc3c2CCN1c1nccc(C(F)(F)F)n1 ; Cc1nc(C)nc(N2CCc3c([nH]c4ccc(Cl)cc34)C2c2ccc(C)c(F)c2F)n1]

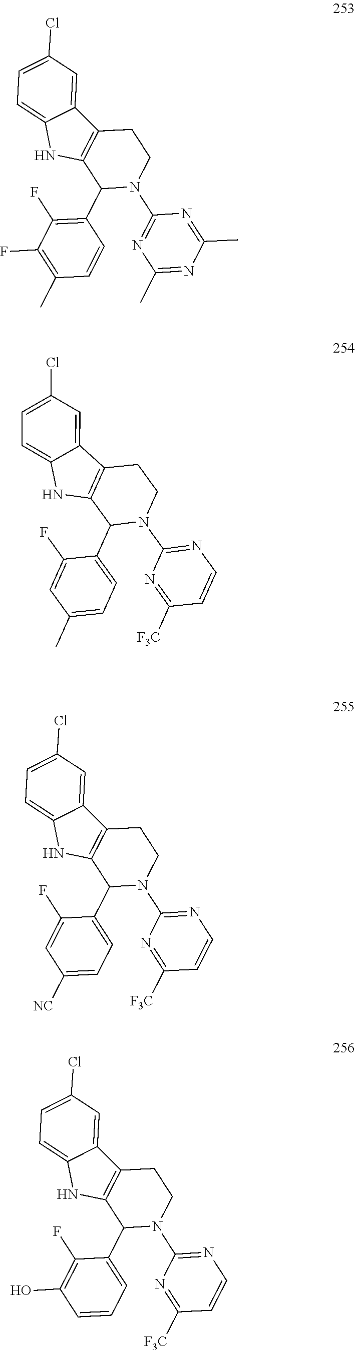 [SMILES: Cc1nc(C)nc(N2CCc3c([nH]c4ccc(Cl)cc34)C2c2ccc(C)c(F)c2F)n1 ; Cc1ccc(C2c3[nH]c4ccc(Cl)cc4c3CCN2c2nccc(C(F)(F)F)n2)c(F)c1 ; N#Cc1ccc(C2c3[nH]c4ccc(Cl)cc4c3CCN2c2nccc(C(F)(F)F)n2)c(F)c1 ; Oc1cccc(C2c3[nH]c4ccc(Cl)cc4c3CCN2c2nccc(C(F)(F)F)n2)c1F]

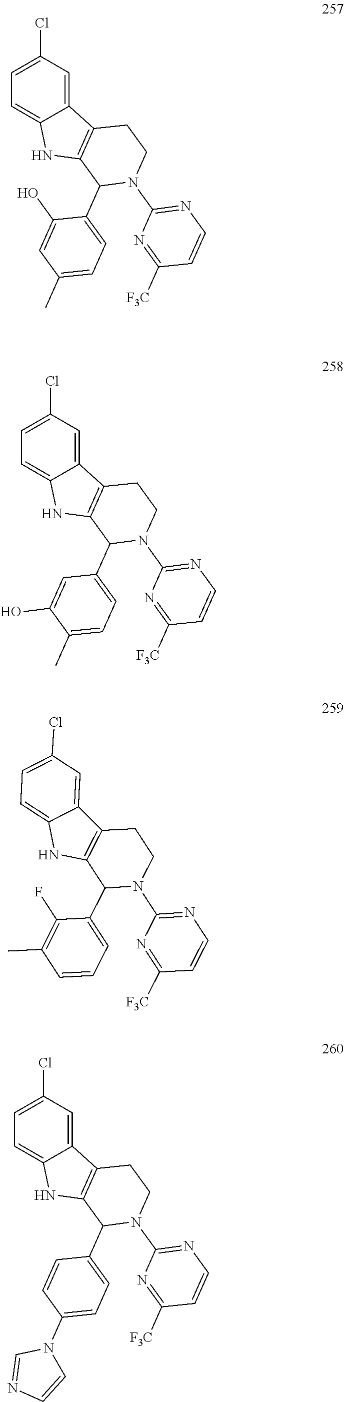 [SMILES: Cc1ccc(C2c3[nH]c4ccc(Cl)cc4c3CCN2c2nccc(C(F)(F)F)n2)c(O)c1 ; Cc1ccc(C2c3[nH]c4ccc(Cl)cc4c3CCN2c2nccc(C(F)(F)F)n2)cc1O ; Cc1cccc(C2c3[nH]c4ccc(Cl)cc4c3CCN2c2nccc(C(F)(F)F)n2)c1F ; FC(F)(F)c1ccnc(N2CCc3c([nH]c4ccc(Cl)cc34)C2c2ccc(-n3ccnc3)cc2)n1]

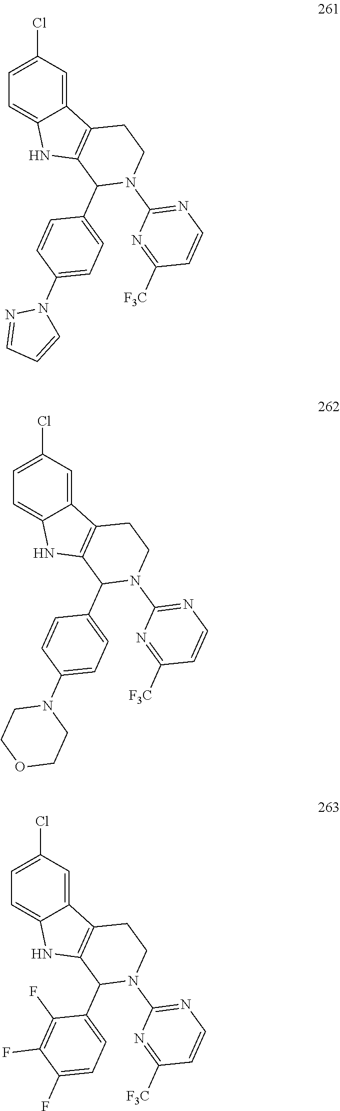 [SMILES: FC(F)(F)c1ccnc(N2CCc3c([nH]c4ccc(Cl)cc34)C2c2ccc(-n3cccn3)cc2)n1 ; FC(F)(F)c1ccnc(N2CCc3c([nH]c4ccc(Cl)cc34)C2c2ccc(N3CCOCC3)cc2)n1 ; Fc1ccc(C2c3[nH]c4ccc(Cl)cc4c3CCN2c2nccc(C(F)(F)F)n2)c(F)c1F]

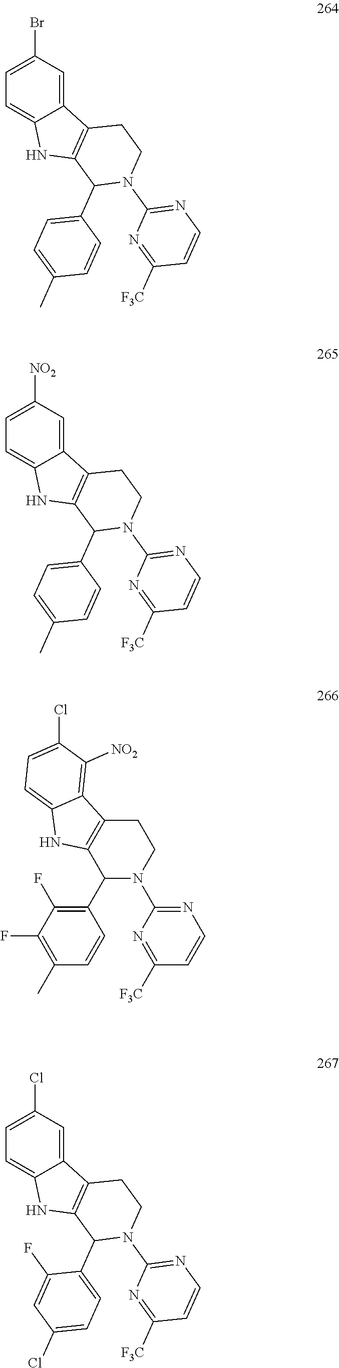 [SMILES: Cc1ccc(C2c3[nH]c4ccc(Br)cc4c3CCN2c2nccc(C(F)(F)F)n2)cc1 ; Cc1ccc(C2c3[nH]c4ccc([N+](=O)[O-])cc4c3CCN2c2nccc(C(F)(F)F)n2)cc1 ; Cc1ccc(C2c3[nH]c4ccc(Cl)c([N+](=O)[O-])c4c3CCN2c2nccc(C(F)(F)F)n2)c(F)c1F ; Fc1cc(Cl)ccc1C1c2[nH]c3ccc(Cl)cc3c2CCN1c1nccc(C(F)(F)F)n1]

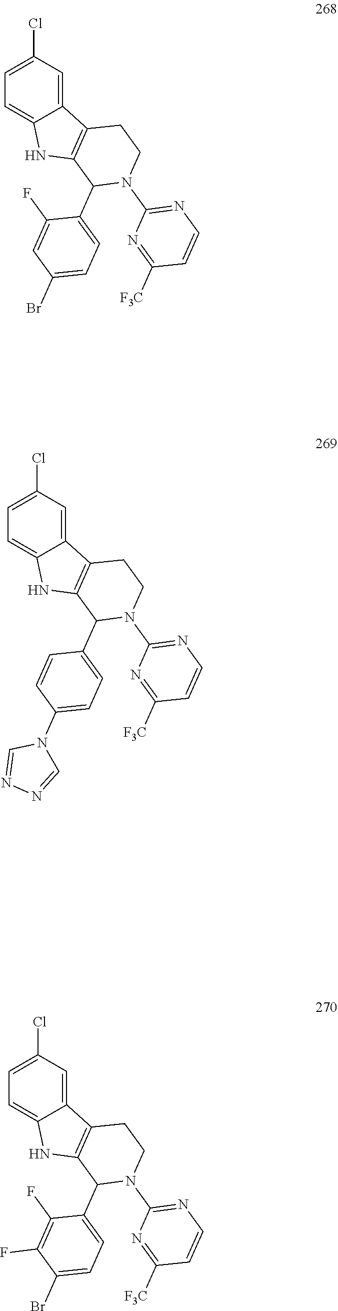 [SMILES: Fc1cc(Br)ccc1C1c2[nH]c3ccc(Cl)cc3c2CCN1c1nccc(C(F)(F)F)n1 ; FC(F)(F)c1ccnc(N2CCc3c([nH]c4ccc(Cl)cc34)C2c2ccc(-n3cnnc3)cc2)n1 ; Fc1c(Br)ccc(C2c3[nH]c4ccc(Cl)cc4c3CCN2c2nccc(C(F)(F)F)n2)c1F]

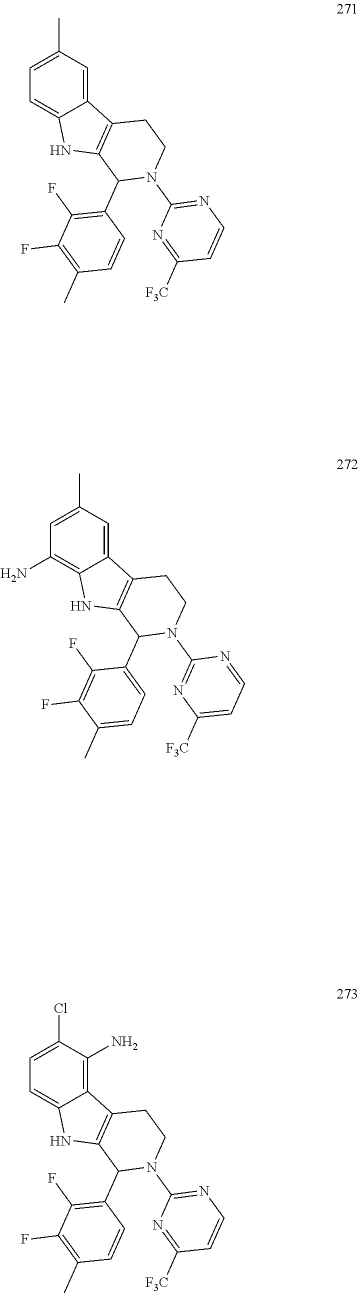 [SMILES: Cc1ccc2[nH]c3c(c2c1)CCN(c1nccc(C(F)(F)F)n1)C3c1ccc(C)c(F)c1F ; Cc1cc(N)c2[nH]c3c(c2c1)CCN(c1nccc(C(F)(F)F)n1)C3c1ccc(C)c(F)c1F ; Cc1ccc(C2c3[nH]c4ccc(Cl)c(N)c4c3CCN2c2nccc(C(F)(F)F)n2)c(F)c1F]

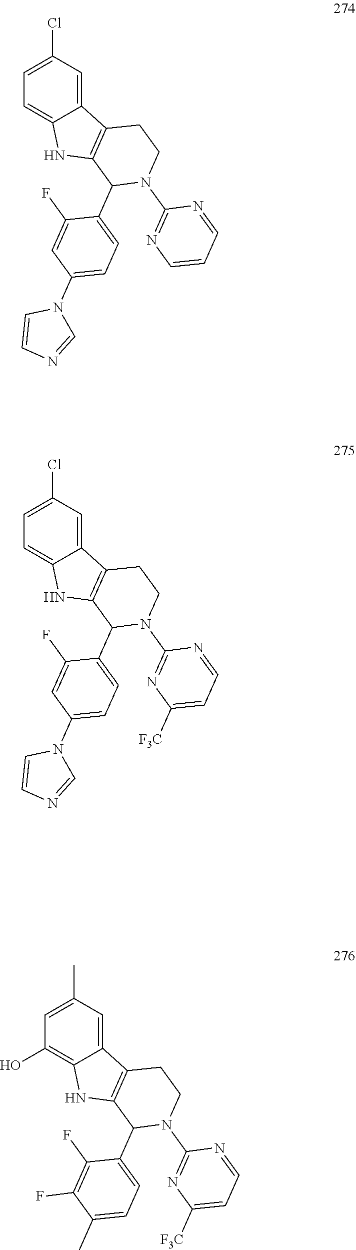 [SMILES: Fc1cc(-n2ccnc2)ccc1C1c2[nH]c3ccc(Cl)cc3c2CCN1c1ncccn1 ; Fc1cc(-n2ccnc2)ccc1C1c2[nH]c3ccc(Cl)cc3c2CCN1c1nccc(C(F)(F)F)n1 ; Cc1cc(O)c2[nH]c3c(c2c1)CCN(c1nccc(C(F)(F)F)n1)C3c1ccc(C)c(F)c1F]

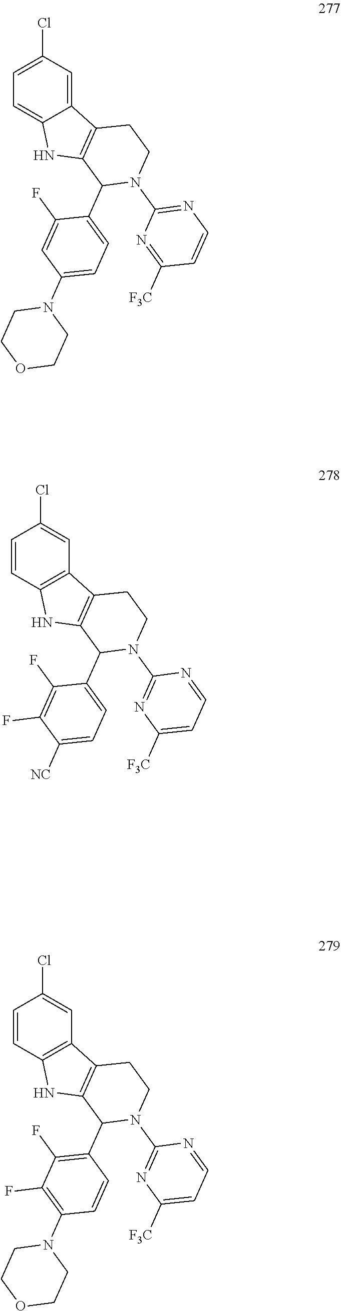 [SMILES: Fc1cc(N2CCOCC2)ccc1C1c2[nH]c3ccc(Cl)cc3c2CCN1c1nccc(C(F)(F)F)n1 ; N#Cc1ccc(C2c3[nH]c4ccc(Cl)cc4c3CCN2c2nccc(C(F)(F)F)n2)c(F)c1F ; Fc1c(C2c3[nH]c4ccc(Cl)cc4c3CCN2c2nccc(C(F)(F)F)n2)ccc(N2CCOCC2)c1F]

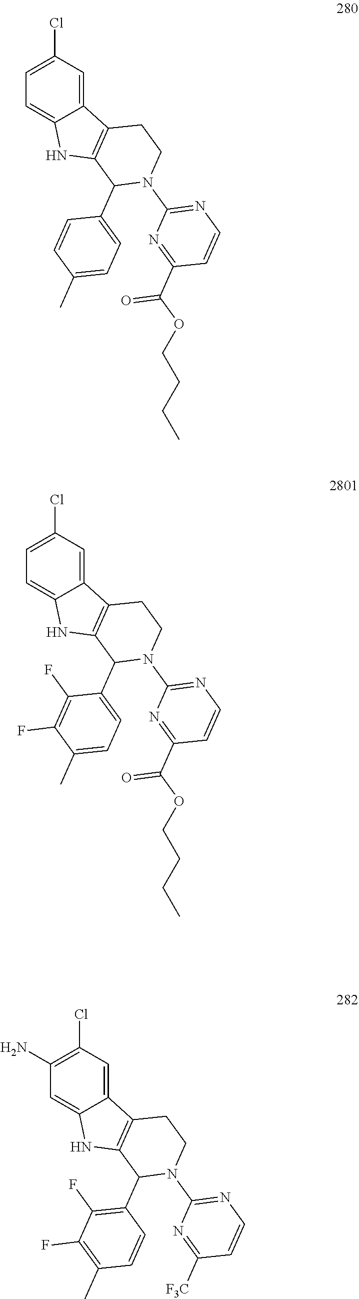 [SMILES: CCCCOC(=O)c1ccnc(N2CCc3c([nH]c4ccc(Cl)cc34)C2c2ccc(C)cc2)n1 ; CCCCOC(=O)c1ccnc(N2CCc3c([nH]c4ccc(Cl)cc34)C2c2ccc(C)c(F)c2F)n1 ; Cc1ccc(C2c3[nH]c4cc(N)c(Cl)cc4c3CCN2c2nccc(C(F)(F)F)n2)c(F)c1F]

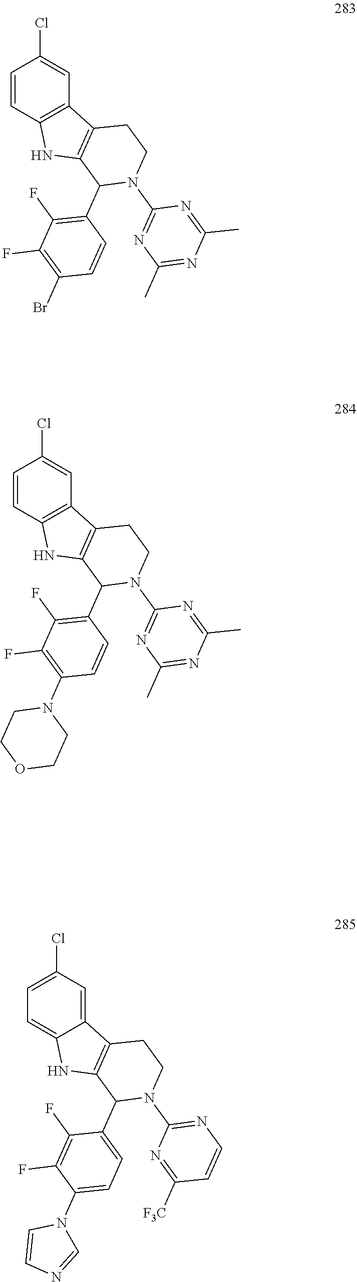 [SMILES: Cc1nc(C)nc(N2CCc3c([nH]c4ccc(Cl)cc34)C2c2ccc(Br)c(F)c2F)n1 ; Cc1nc(C)nc(N2CCc3c([nH]c4ccc(Cl)cc34)C2c2ccc(N3CCOCC3)c(F)c2F)n1 ; Fc1c(C2c3[nH]c4ccc(Cl)cc4c3CCN2c2nccc(C(F)(F)F)n2)ccc(-n2ccnc2)c1F]

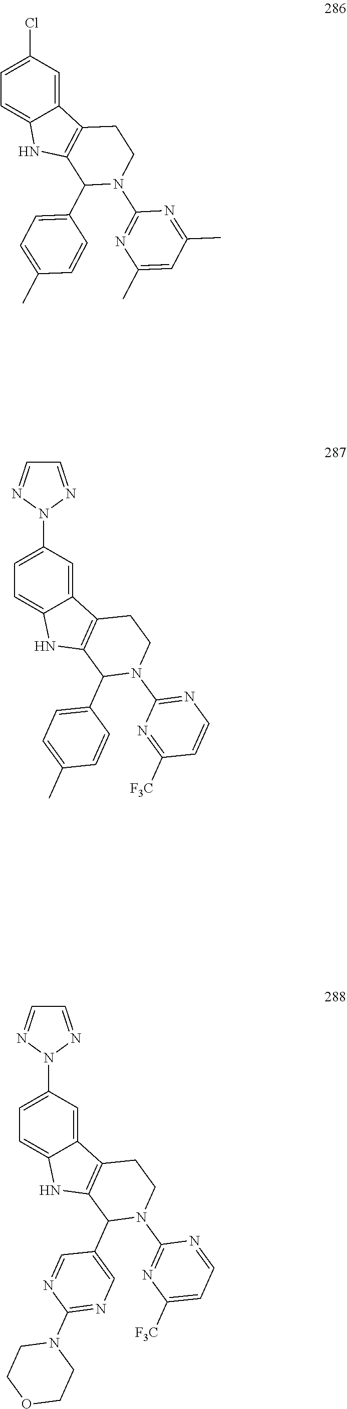 [SMILES: Cc1ccc(C2c3[nH]c4ccc(Cl)cc4c3CCN2c2nc(C)cc(C)n2)cc1 ; Cc1ccc(C2c3[nH]c4ccc(-n5nccn5)cc4c3CCN2c2nccc(C(F)(F)F)n2)cc1 ; FC(F)(F)c1ccnc(N2CCc3c([nH]c4ccc(-n5nccn5)cc34)C2c2cnc(N3CCOCC3)nc2)n1]

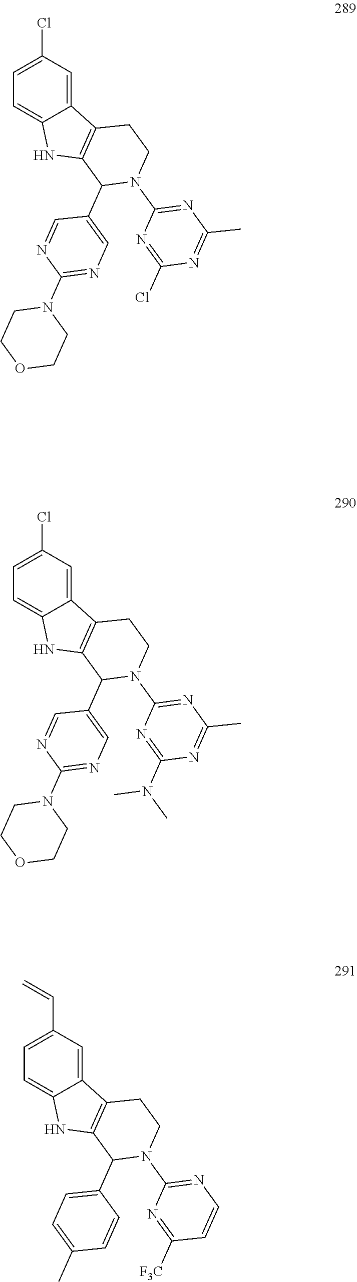 [SMILES: Cc1nc(Cl)nc(N2CCc3c([nH]c4ccc(Cl)cc34)C2c2cnc(N3CCOCC3)nc2)n1 ; Cc1nc(N(C)C)nc(N2CCc3c([nH]c4ccc(Cl)cc34)C2c2cnc(N3CCOCC3)nc2)n1 ; C=Cc1ccc2[nH]c3c(c2c1)CCN(c1nccc(C(F)(F)F)n1)C3c1ccc(C)cc1]

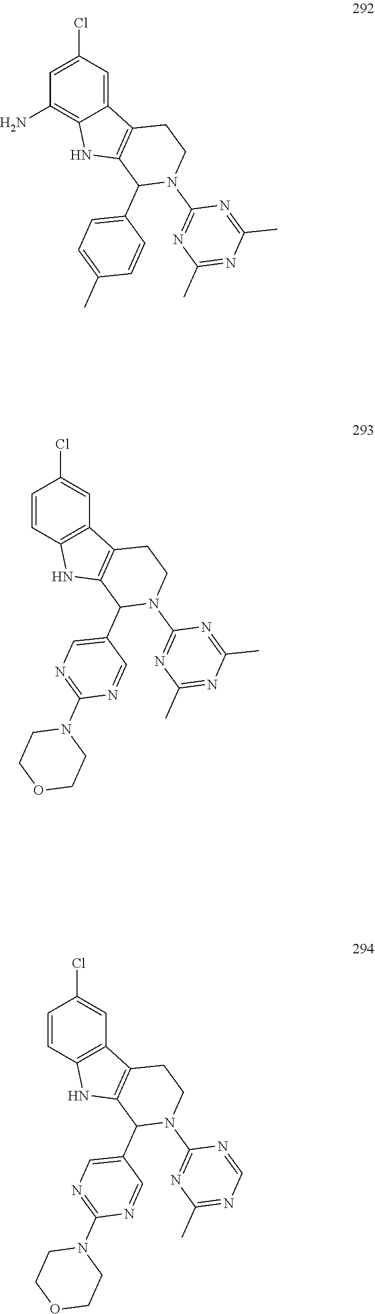 [SMILES: Cc1ccc(C2c3[nH]c4c(N)cc(Cl)cc4c3CCN2c2nc(C)nc(C)n2)cc1 ; Cc1nc(C)nc(N2CCc3c([nH]c4ccc(Cl)cc34)C2c2cnc(N3CCOCC3)nc2)n1 ; Cc1ncnc(N2CCc3c([nH]c4ccc(Cl)cc34)C2c2cnc(N3CCOCC3)nc2)n1]

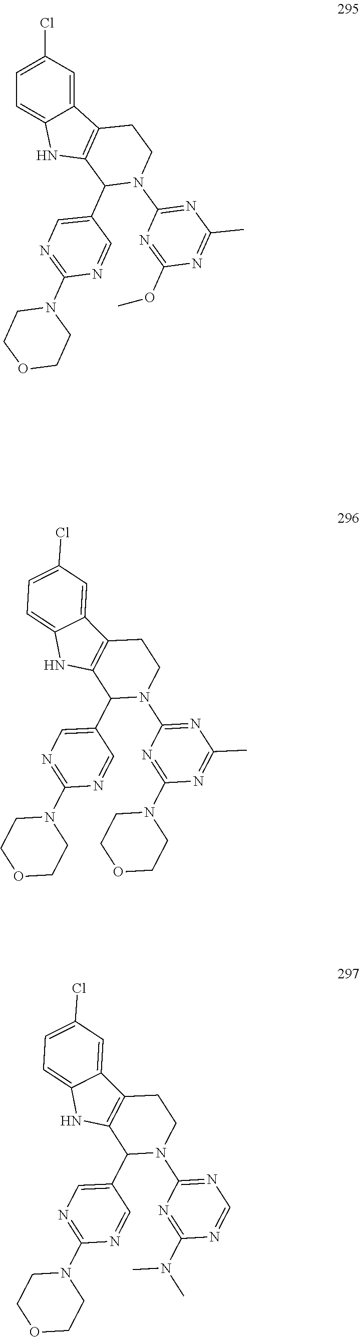 [SMILES: COc1nc(C)nc(N2CCc3c([nH]c4ccc(Cl)cc34)C2c2cnc(N3CCOCC3)nc2)n1 ; Cc1nc(N2CCOCC2)nc(N2CCc3c([nH]c4ccc(Cl)cc34)C2c2cnc(N3CCOCC3)nc2)n1 ; CN(C)c1ncnc(N2CCc3c([nH]c4ccc(Cl)cc34)C2c2cnc(N3CCOCC3)nc2)n1]

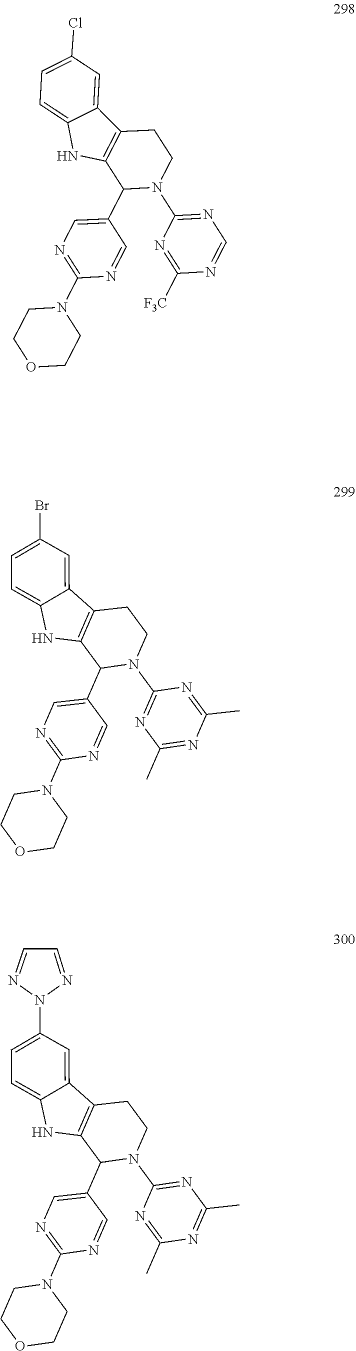 [SMILES: FC(F)(F)c1ncnc(N2CCc3c([nH]c4ccc(Cl)cc34)C2c2cnc(N3CCOCC3)nc2)n1 ; Cc1nc(C)nc(N2CCc3c([nH]c4ccc(Br)cc34)C2c2cnc(N3CCOCC3)nc2)n1 ; Cc1nc(C)nc(N2CCc3c([nH]c4ccc(-n5nccn5)cc34)C2c2cnc(N3CCOCC3)nc2)n1]

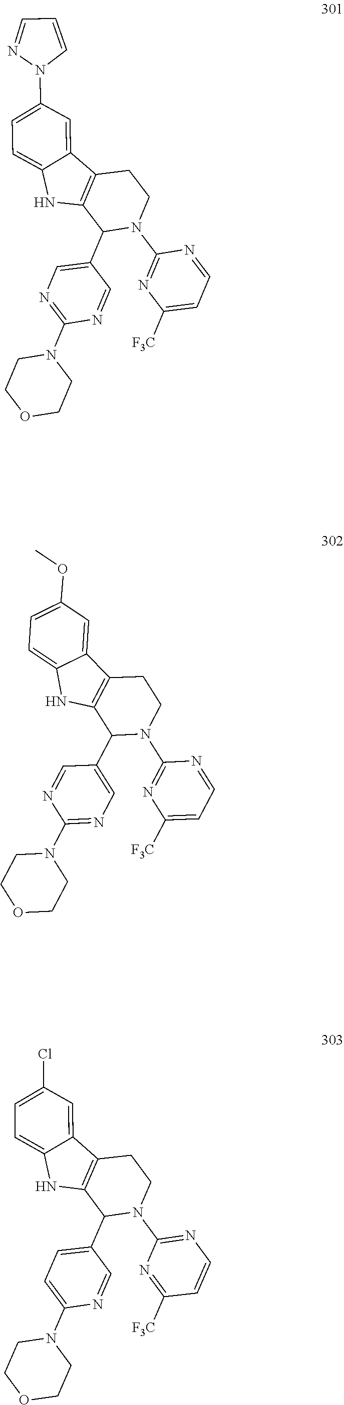 [SMILES: FC(F)(F)c1ccnc(N2CCc3c([nH]c4ccc(-n5cccn5)cc34)C2c2cnc(N3CCOCC3)nc2)n1 ; COc1ccc2[nH]c3c(c2c1)CCN(c1nccc(C(F)(F)F)n1)C3c1cnc(N2CCOCC2)nc1 ; FC(F)(F)c1ccnc(N2CCc3c([nH]c4ccc(Cl)cc34)C2c2ccc(N3CCOCC3)nc2)n1]

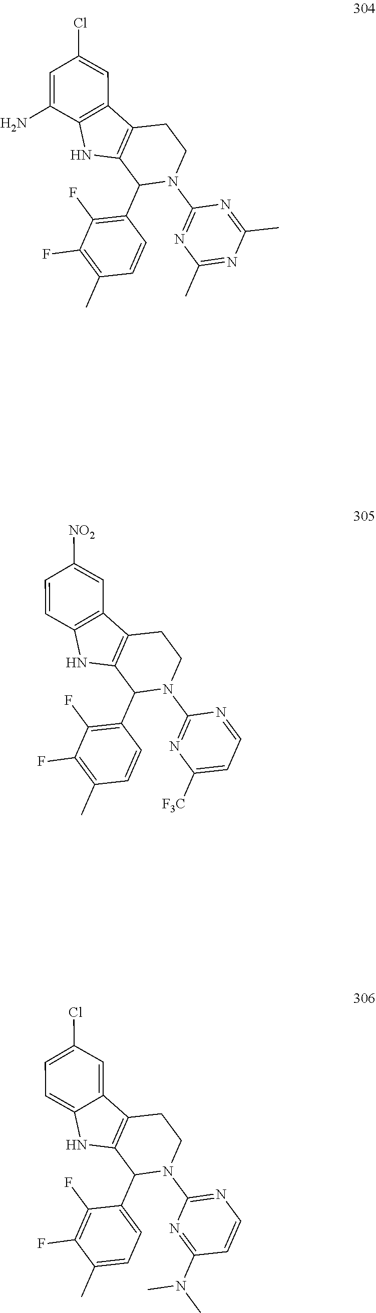 [SMILES: Cc1nc(C)nc(N2CCc3c([nH]c4c(N)cc(Cl)cc34)C2c2ccc(C)c(F)c2F)n1 ; Cc1ccc(C2c3[nH]c4ccc([N+](=O)[O-])cc4c3CCN2c2nccc(C(F)(F)F)n2)c(F)c1F ; Cc1ccc(C2c3[nH]c4ccc(Cl)cc4c3CCN2c2nccc(N(C)C)n2)c(F)c1F]

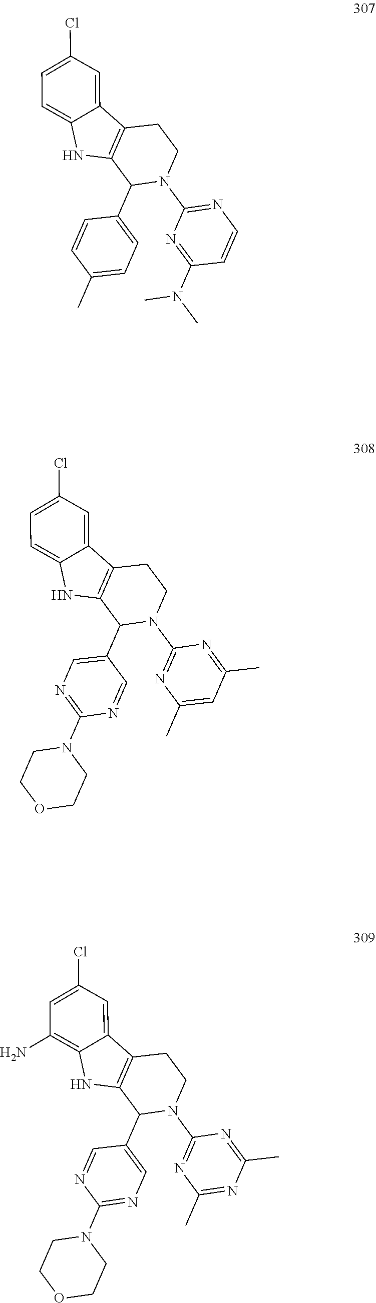 [SMILES: Cc1ccc(C2c3[nH]c4ccc(Cl)cc4c3CCN2c2nccc(N(C)C)n2)cc1 ; Cc1cc(C)nc(N2CCc3c([nH]c4ccc(Cl)cc34)C2c2cnc(N3CCOCC3)nc2)n1 ; Cc1nc(C)nc(N2CCc3c([nH]c4c(N)cc(Cl)cc34)C2c2cnc(N3CCOCC3)nc2)n1]

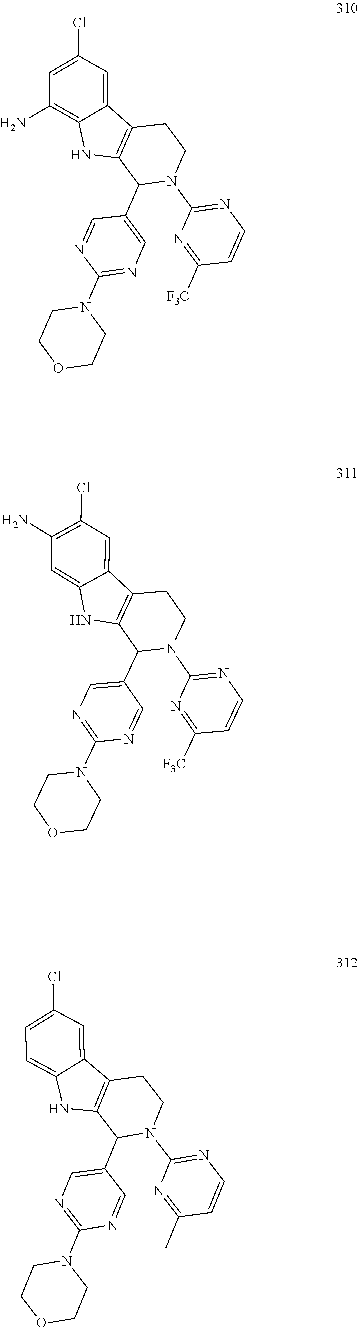 [SMILES: Nc1cc(Cl)cc2c3c([nH]c12)C(c1cnc(N2CCOCC2)nc1)N(c1nccc(C(F)(F)F)n1)CC3 ; Nc1cc2[nH]c3c(c2cc1Cl)CCN(c1nccc(C(F)(F)F)n1)C3c1cnc(N2CCOCC2)nc1 ; Cc1ccnc(N2CCc3c([nH]c4ccc(Cl)cc34)C2c2cnc(N3CCOCC3)nc2)n1]

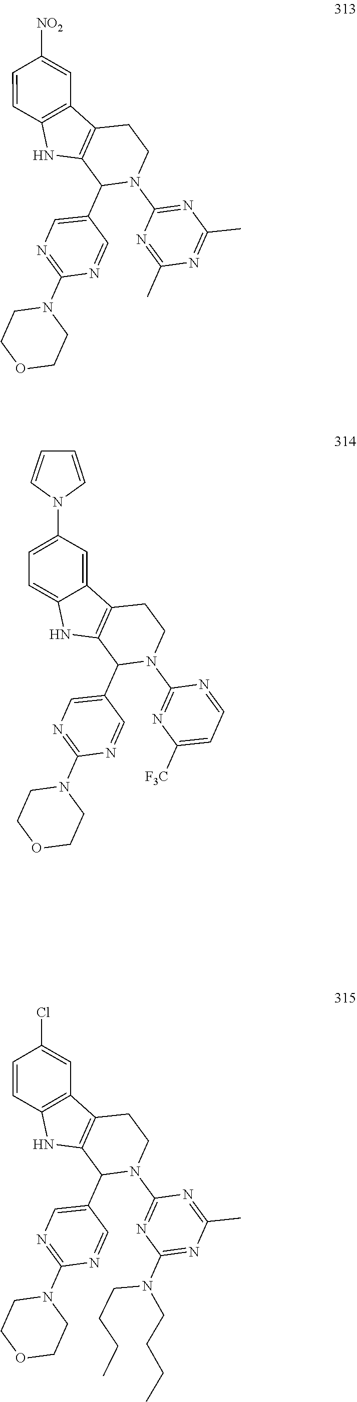 [SMILES: Cc1nc(C)nc(N2CCc3c([nH]c4ccc([N+](=O)[O-])cc34)C2c2cnc(N3CCOCC3)nc2)n1 ; FC(F)(F)c1ccnc(N2CCc3c([nH]c4ccc(-n5cccc5)cc34)C2c2cnc(N3CCOCC3)nc2)n1 ; CCCCN(CCCC)c1nc(C)nc(N2CCc3c([nH]c4ccc(Cl)cc34)C2c2cnc(N3CCOCC3)nc2)n1]

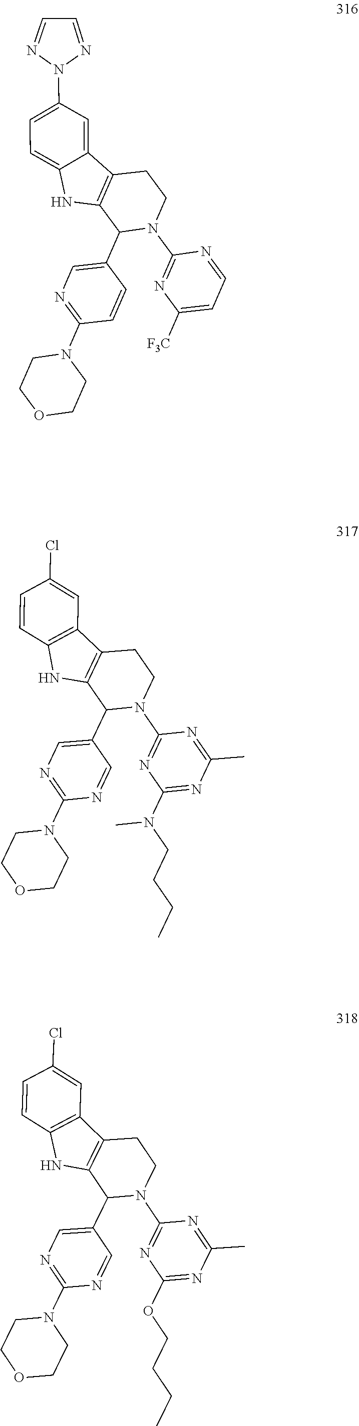 [SMILES: FC(F)(F)c1ccnc(N2CCc3c([nH]c4ccc(-n5nccn5)cc34)C2c2ccc(N3CCOCC3)nc2)n1 ; CCCCN(C)c1nc(C)nc(N2CCc3c([nH]c4ccc(Cl)cc34)C2c2cnc(N3CCOCC3)nc2)n1 ; CCCCOc1nc(C)nc(N2CCc3c([nH]c4ccc(Cl)cc34)C2c2cnc(N3CCOCC3)nc2)n1]

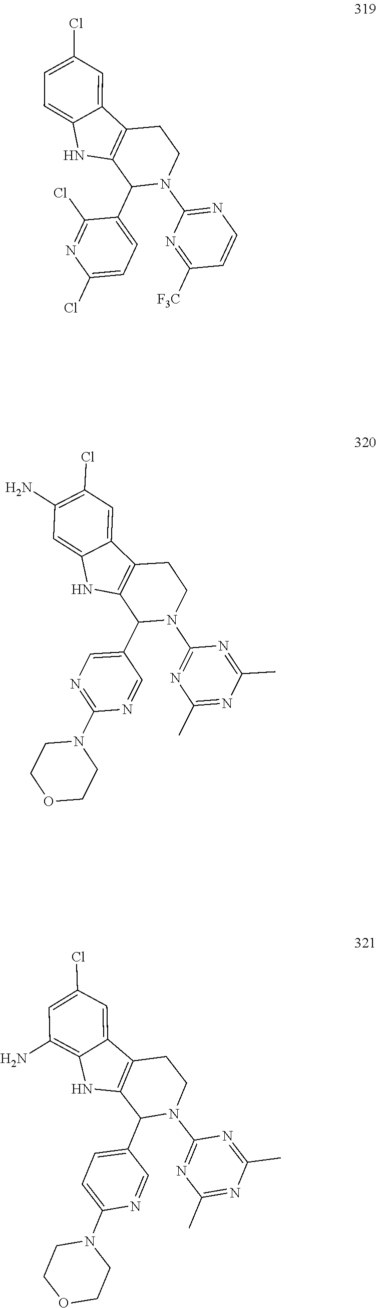 [SMILES: FC(F)(F)c1ccnc(N2CCc3c([nH]c4ccc(Cl)cc34)C2c2ccc(Cl)nc2Cl)n1 ; Cc1nc(C)nc(N2CCc3c([nH]c4cc(N)c(Cl)cc34)C2c2cnc(N3CCOCC3)nc2)n1 ; Cc1nc(C)nc(N2CCc3c([nH]c4c(N)cc(Cl)cc34)C2c2ccc(N3CCOCC3)nc2)n1]

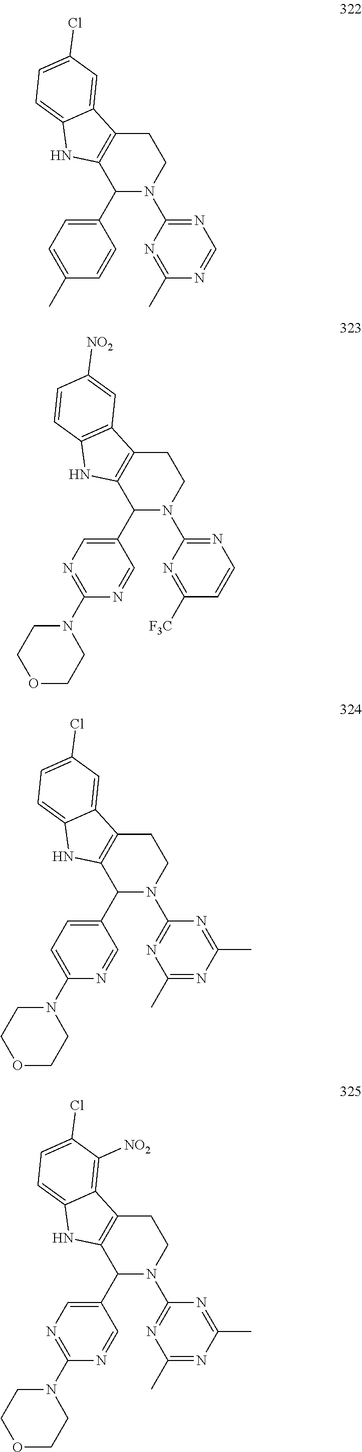

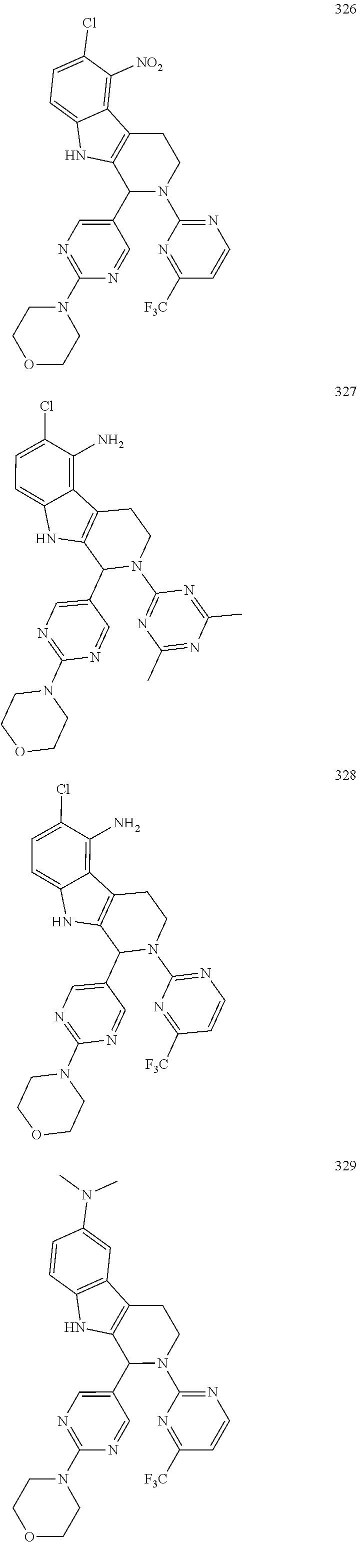

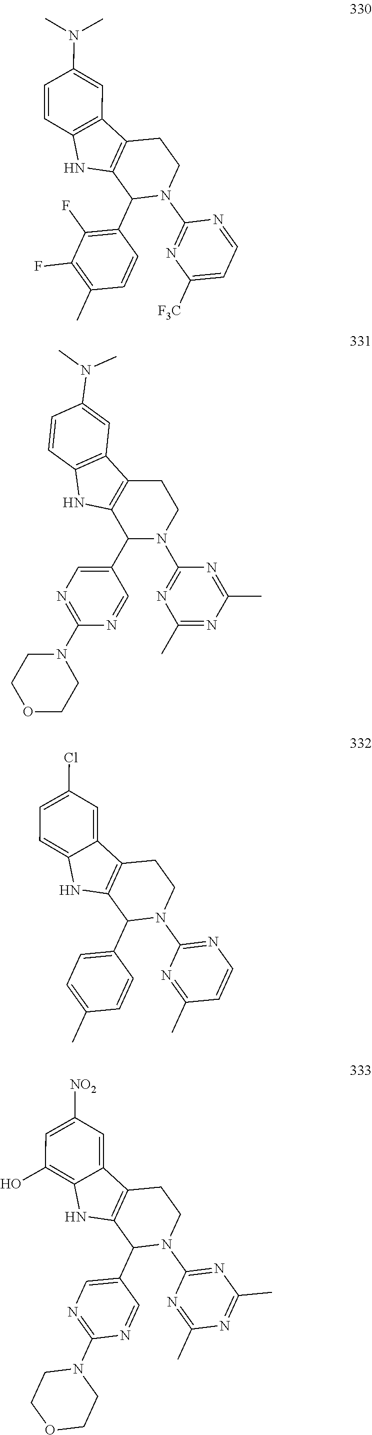

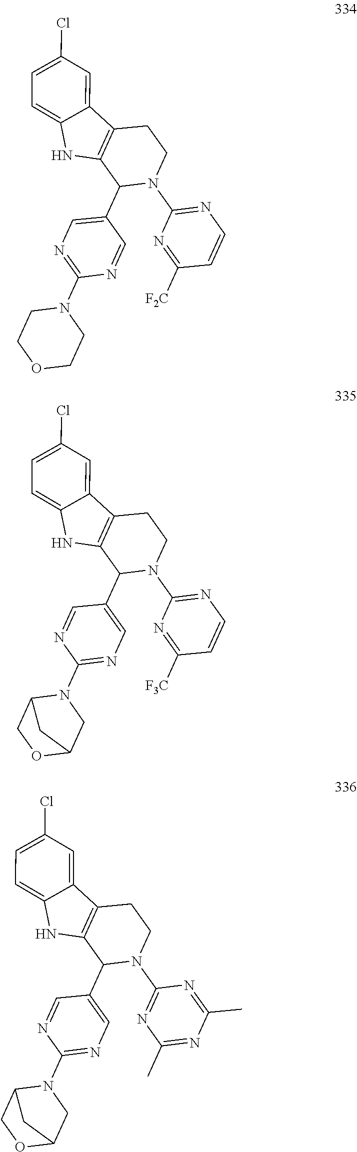

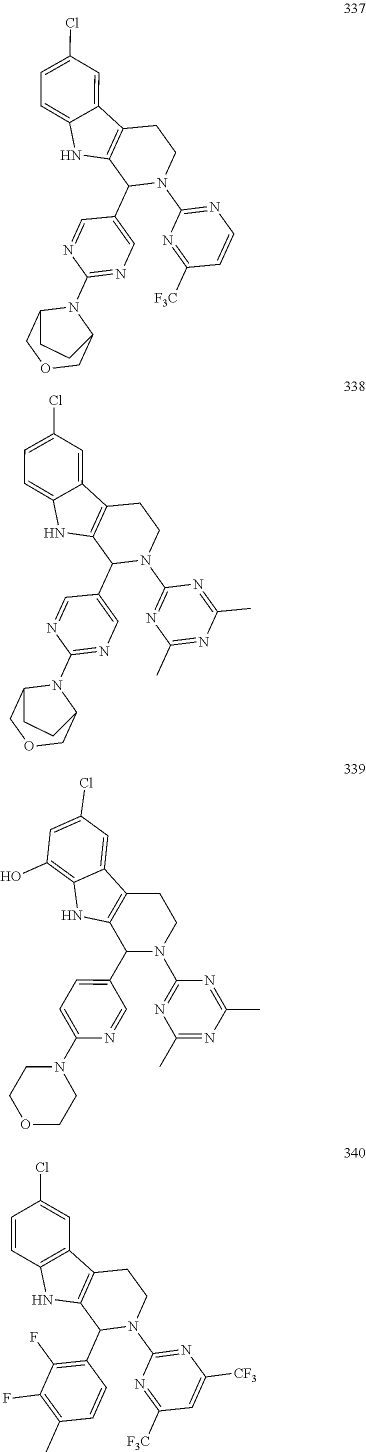

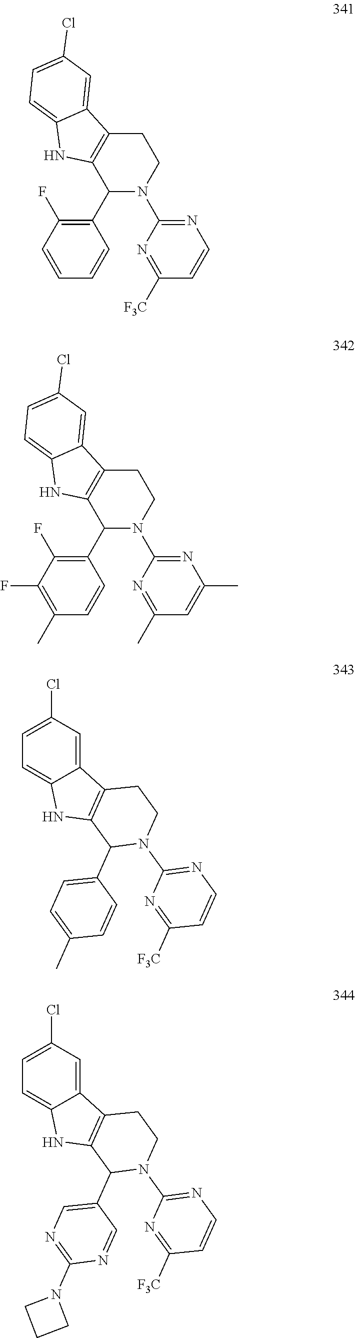

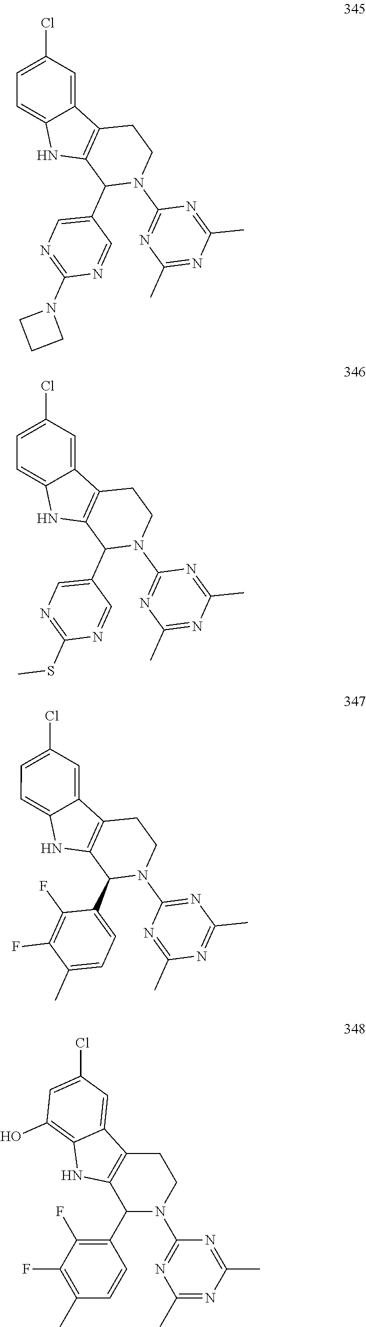

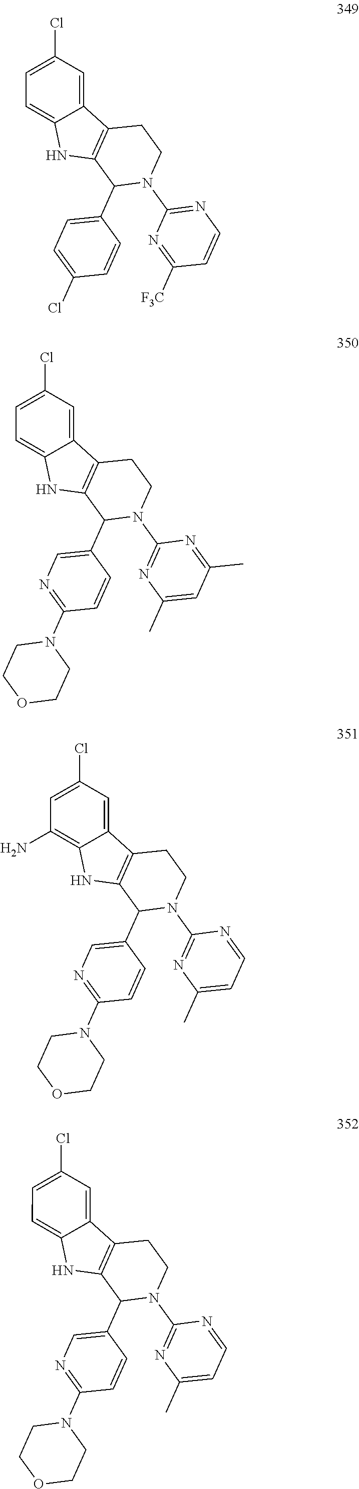

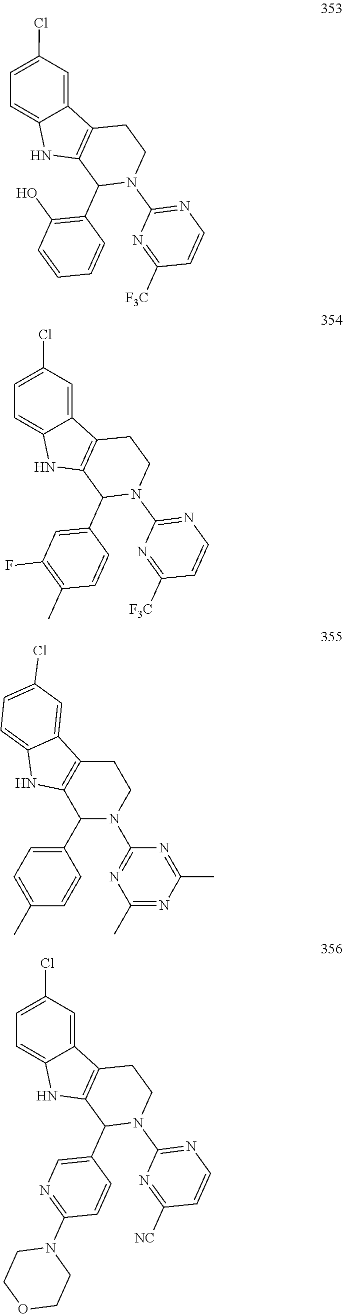

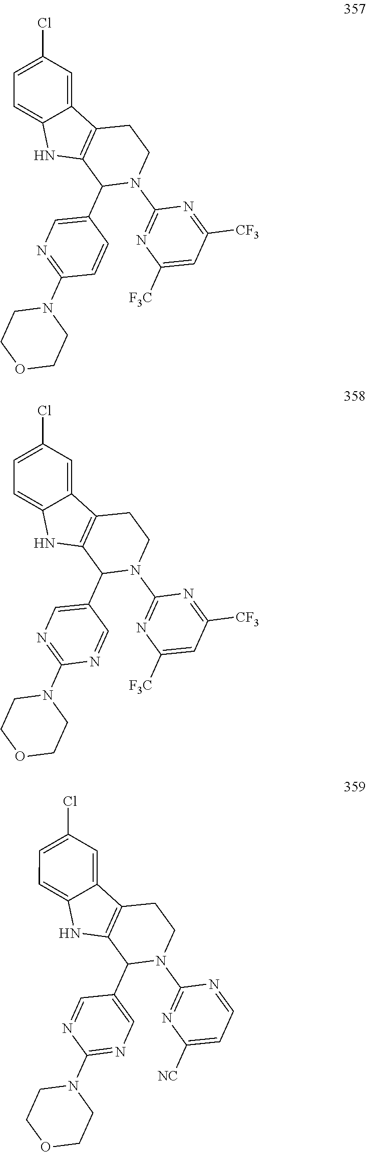

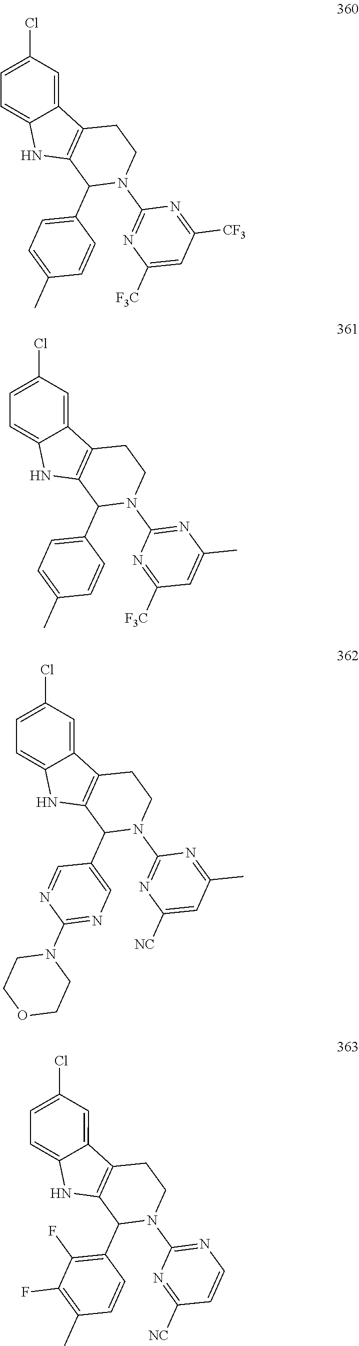

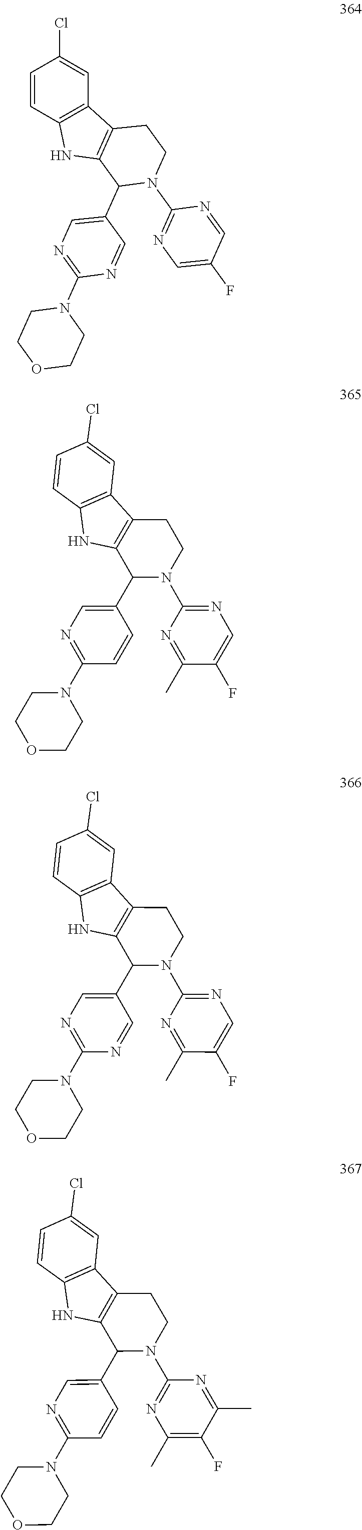

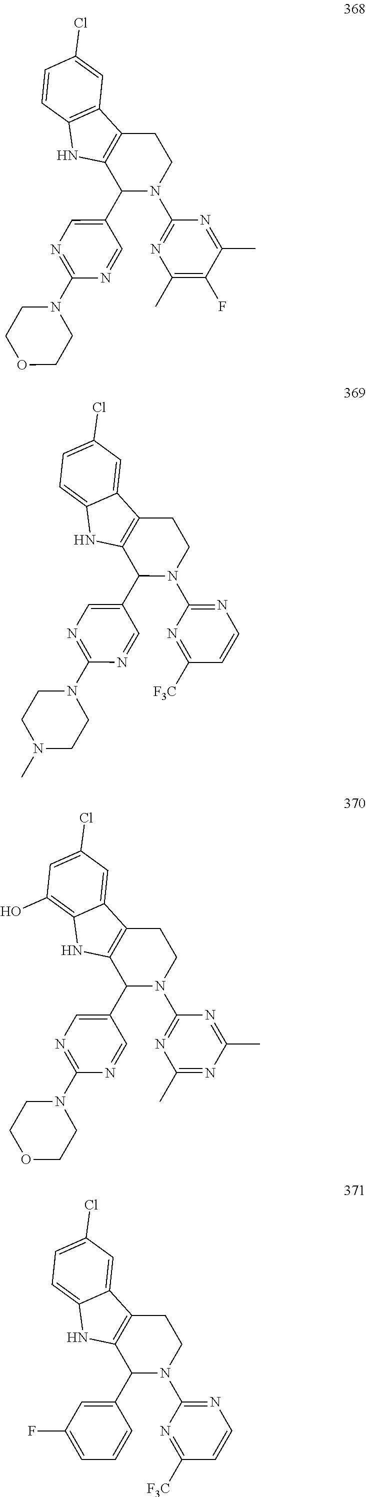

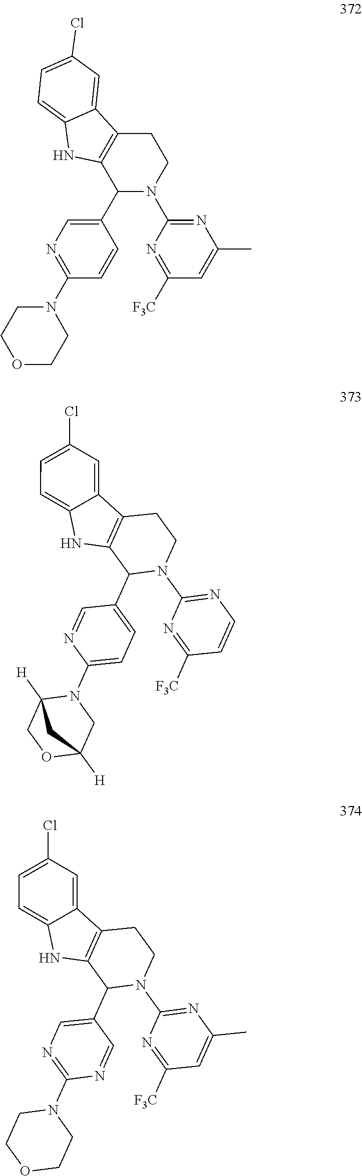 [SMILES: Cc1cc(C(F)(F)F)nc(N2CCc3c([nH]c4ccc(Cl)cc34)C2c2ccc(N3CCOCC3)nc2)n1 ; FC(F)(F)c1ccnc(N2CCc3c([nH]c4ccc(Cl)cc34)C2c2ccc(N3C[C@H]4C[C@@H]3CO4)nc2)n1 ; Cc1cc(C(F)(F)F)nc(N2CCc3c([nH]c4ccc(Cl)cc34)C2c2cnc(N3CCOCC3)nc2)n1]

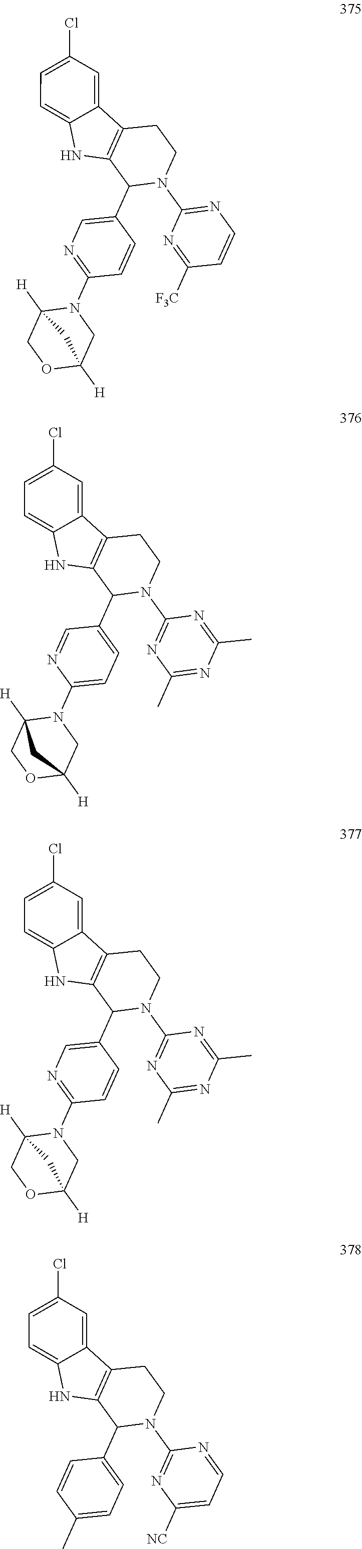 [SMILES: FC(F)(F)c1ccnc(N2CCc3c([nH]c4ccc(Cl)cc34)C2c2ccc(N3C[C@@H]4C[C@H]3CO4)nc2)n1 ; Cc1nc(C)nc(N2CCc3c([nH]c4ccc(Cl)cc34)C2c2ccc(N3C[C@H]4C[C@@H]3CO4)nc2)n1 ; Cc1nc(C)nc(N2CCc3c([nH]c4ccc(Cl)cc34)C2c2ccc(N3C[C@@H]4C[C@H]3CO4)nc2)n1 ; Cc1ccc(C2c3[nH]c4ccc(Cl)cc4c3CCN2c2nccc(C#N)n2)cc1]

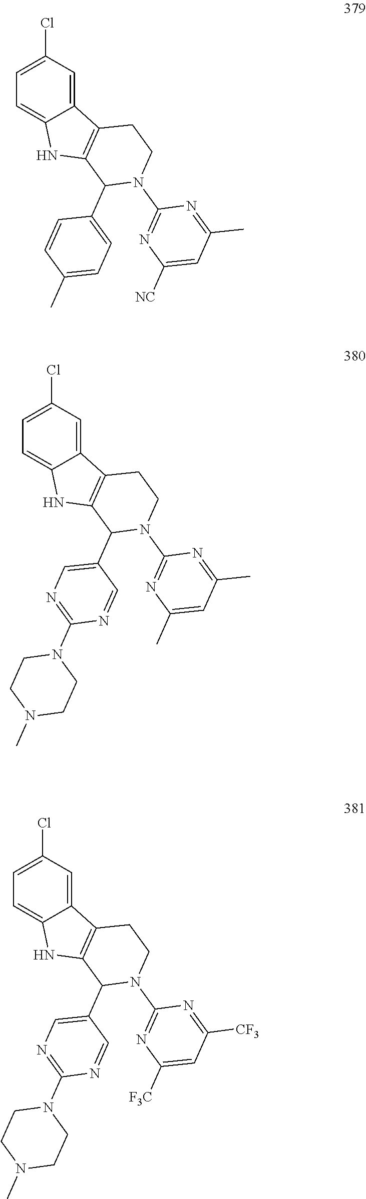 [SMILES: Cc1ccc(C2c3[nH]c4ccc(Cl)cc4c3CCN2c2nc(C)cc(C#N)n2)cc1 ; Cc1cc(C)nc(N2CCc3c([nH]c4ccc(Cl)cc34)C2c2cnc(N3CCN(C)CC3)nc2)n1 ; CN1CCN(c2ncc(C3c4[nH]c5ccc(Cl)cc5c4CCN3c3nc(C(F)(F)F)cc(C(F)(F)F)n3)cn2)CC1]

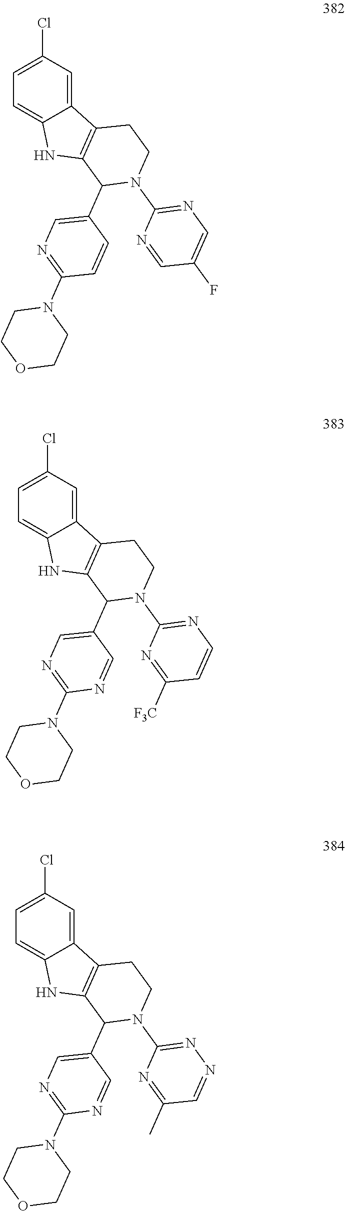 [SMILES: Fc1cnc(N2CCc3c([nH]c4ccc(Cl)cc34)C2c2ccc(N3CCOCC3)nc2)nc1 ; FC(F)(F)c1ccnc(N2CCc3c([nH]c4ccc(Cl)cc34)C2c2cnc(N3CCOCC3)nc2)n1 ; Cc1cnnc(N2CCc3c([nH]c4ccc(Cl)cc34)C2c2cnc(N3CCOCC3)nc2)n1]

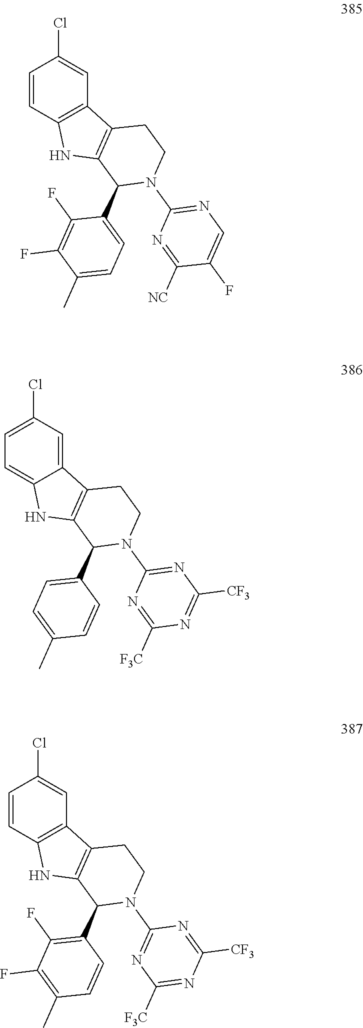 [SMILES: Cc1ccc([C@H]2c3[nH]c4ccc(Cl)cc4c3CCN2c2ncc(F)c(C#N)n2)c(F)c1F ; Cc1ccc([C@H]2c3[nH]c4ccc(Cl)cc4c3CCN2c2nc(C(F)(F)F)nc(C(F)(F)F)n2)cc1 ; Cc1ccc([C@H]2c3[nH]c4ccc(Cl)cc4c3CCN2c2nc(C(F)(F)F)nc(C(F)(F)F)n2)c(F)c1F]

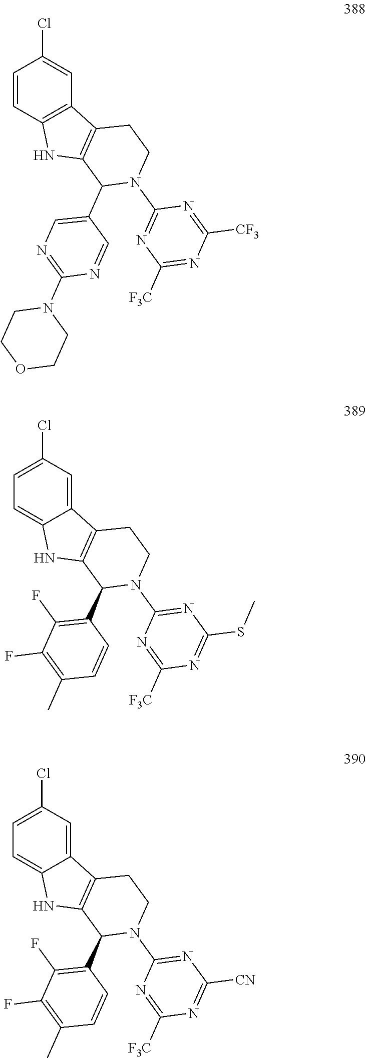 [SMILES: FC(F)(F)c1nc(N2CCc3c([nH]c4ccc(Cl)cc34)C2c2cnc(N3CCOCC3)nc2)nc(C(F)(F)F)n1 ; CSc1nc(N2CCc3c([nH]c4ccc(Cl)cc34)[C@@H]2c2ccc(C)c(F)c2F)nc(C(F)(F)F)n1 ; Cc1ccc([C@H]2c3[nH]c4ccc(Cl)cc4c3CCN2c2nc(C#N)nc(C(F)(F)F)n2)c(F)c1F]

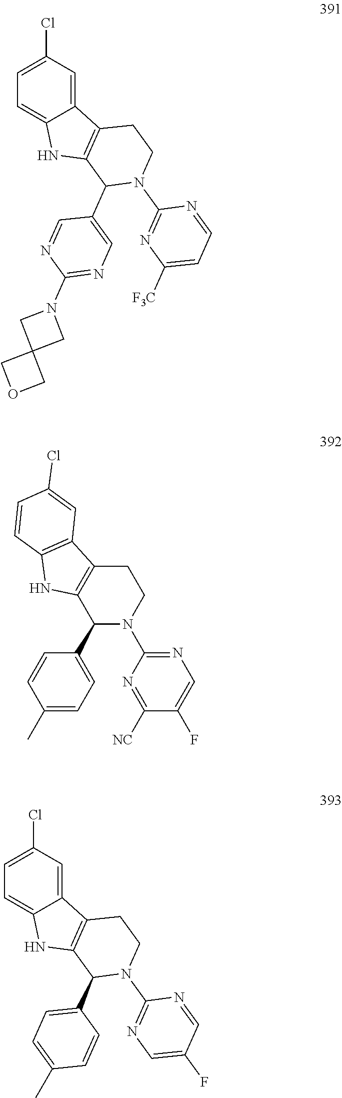 [SMILES: FC(F)(F)c1ccnc(N2CCc3c([nH]c4ccc(Cl)cc34)C2c2cnc(N3CC4(COC4)C3)nc2)n1 ; Cc1ccc([C@H]2c3[nH]c4ccc(Cl)cc4c3CCN2c2ncc(F)c(C#N)n2)cc1 ; Cc1ccc([C@H]2c3[nH]c4ccc(Cl)cc4c3CCN2c2ncc(F)cn2)cc1]

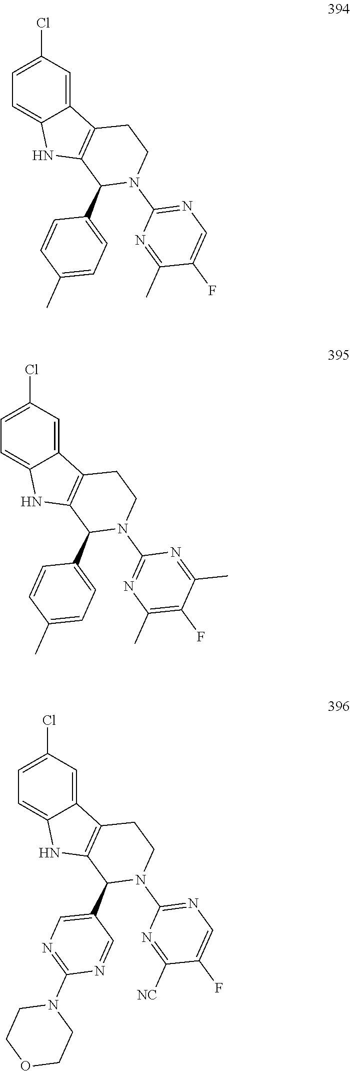 [SMILES: Cc1ccc([C@H]2c3[nH]c4ccc(Cl)cc4c3CCN2c2ncc(F)c(C)n2)cc1 ; Cc1ccc([C@H]2c3[nH]c4ccc(Cl)cc4c3CCN2c2nc(C)c(F)c(C)n2)cc1 ; N#Cc1nc(N2CCc3c([nH]c4ccc(Cl)cc34)[C@@H]2c2cnc(N3CCOCC3)nc2)ncc1F]

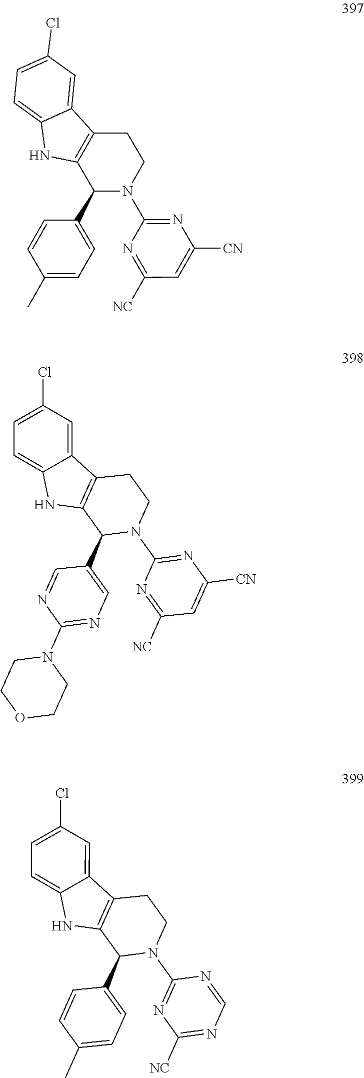 [SMILES: Cc1ccc([C@H]2c3[nH]c4ccc(Cl)cc4c3CCN2c2nc(C#N)cc(C#N)n2)cc1 ; N#Cc1cc(C#N)nc(N2CCc3c([nH]c4ccc(Cl)cc34)[C@@H]2c2cnc(N3CCOCC3)nc2)n1 ; Cc1ccc([C@H]2c3[nH]c4ccc(Cl)cc4c3CCN2c2ncnc(C#N)n2)cc1]

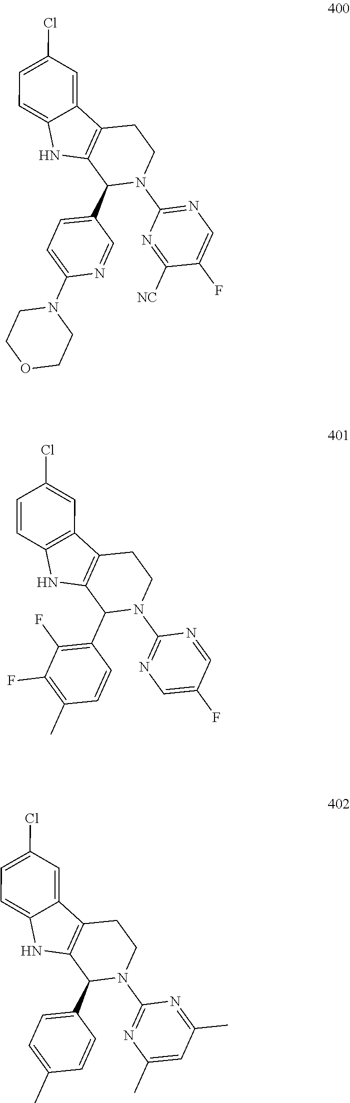 [SMILES: N#Cc1nc(N2CCc3c([nH]c4ccc(Cl)cc34)[C@@H]2c2ccc(N3CCOCC3)nc2)ncc1F ; Cc1ccc(C2c3[nH]c4ccc(Cl)cc4c3CCN2c2ncc(F)cn2)c(F)c1F ; Cc1ccc([C@H]2c3[nH]c4ccc(Cl)cc4c3CCN2c2nc(C)cc(C)n2)cc1]

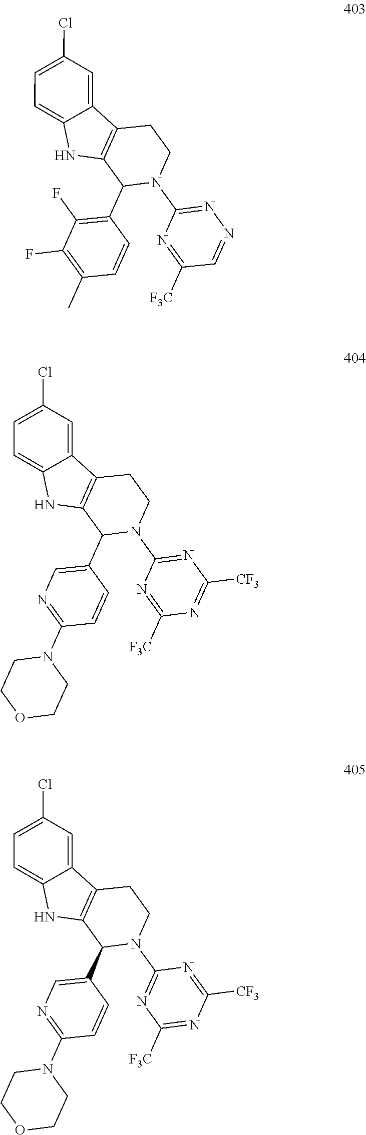 [SMILES: Cc1ccc(C2c3[nH]c4ccc(Cl)cc4c3CCN2c2nncc(C(F)(F)F)n2)c(F)c1F ; FC(F)(F)c1nc(N2CCc3c([nH]c4ccc(Cl)cc34)C2c2ccc(N3CCOCC3)nc2)nc(C(F)(F)F)n1 ; FC(F)(F)c1nc(N2CCc3c([nH]c4ccc(Cl)cc34)[C@@H]2c2ccc(N3CCOCC3)nc2)nc(C(F)(F)F)n1]

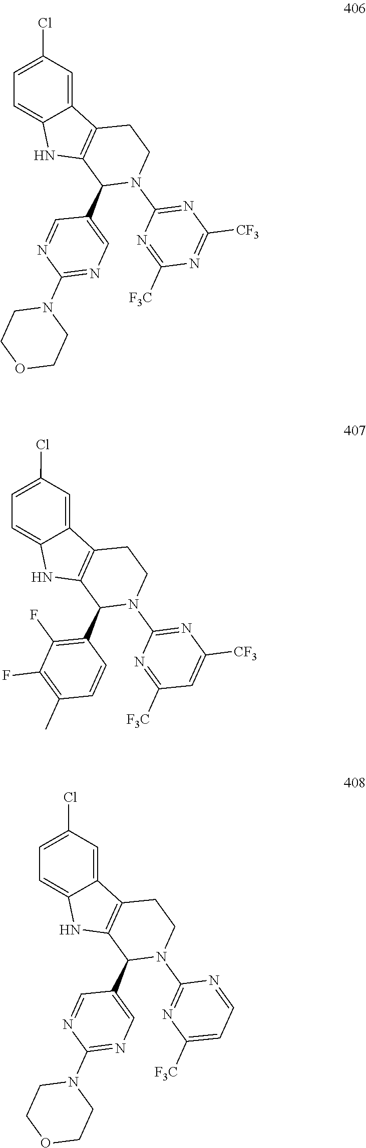 [SMILES: FC(F)(F)c1nc(N2CCc3c([nH]c4ccc(Cl)cc34)[C@@H]2c2cnc(N3CCOCC3)nc2)nc(C(F)(F)F)n1 ; Cc1ccc([C@H]2c3[nH]c4ccc(Cl)cc4c3CCN2c2nc(C(F)(F)F)cc(C(F)(F)F)n2)c(F)c1F ; FC(F)(F)c1ccnc(N2CCc3c([nH]c4ccc(Cl)cc34)[C@@H]2c2cnc(N3CCOCC3)nc2)n1]

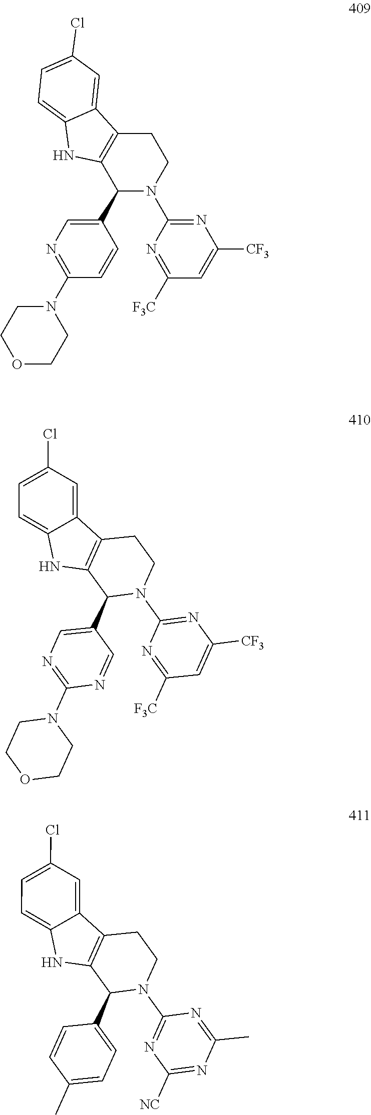 [SMILES: FC(F)(F)c1cc(C(F)(F)F)nc(N2CCc3c([nH]c4ccc(Cl)cc34)[C@@H]2c2ccc(N3CCOCC3)nc2)n1 ; FC(F)(F)c1cc(C(F)(F)F)nc(N2CCc3c([nH]c4ccc(Cl)cc34)[C@@H]2c2cnc(N3CCOCC3)nc2)n1 ; Cc1ccc([C@H]2c3[nH]c4ccc(Cl)cc4c3CCN2c2nc(C)nc(C#N)n2)cc1]

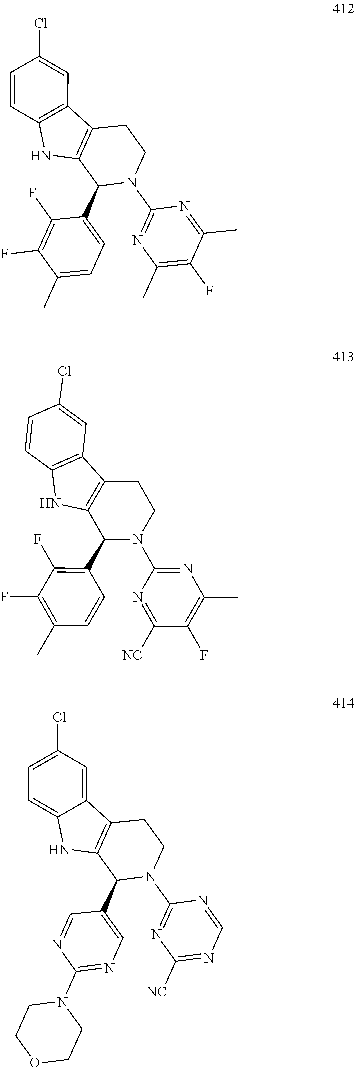 [SMILES: Cc1ccc([C@H]2c3[nH]c4ccc(Cl)cc4c3CCN2c2nc(C)c(F)c(C)n2)c(F)c1F ; Cc1ccc([C@H]2c3[nH]c4ccc(Cl)cc4c3CCN2c2nc(C)c(F)c(C#N)n2)c(F)c1F ; N#Cc1ncnc(N2CCc3c([nH]c4ccc(Cl)cc34)[C@@H]2c2cnc(N3CCOCC3)nc2)n1]

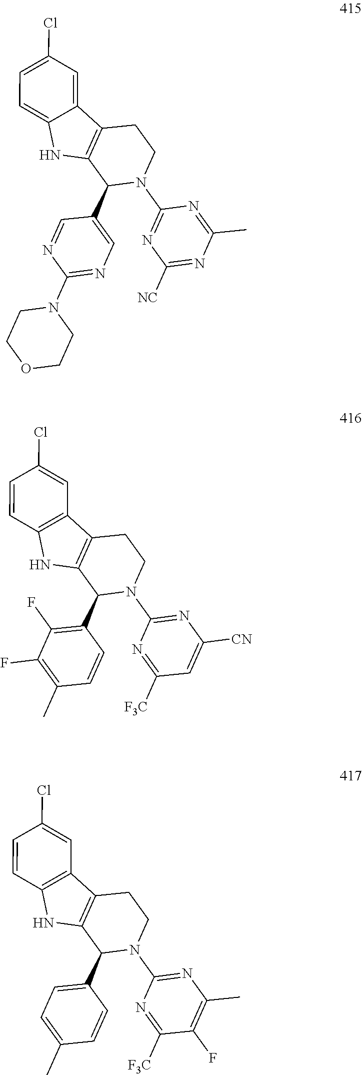 [SMILES: Cc1nc(C#N)nc(N2CCc3c([nH]c4ccc(Cl)cc34)[C@@H]2c2cnc(N3CCOCC3)nc2)n1 ; Cc1ccc([C@H]2c3[nH]c4ccc(Cl)cc4c3CCN2c2nc(C#N)cc(C(F)(F)F)n2)c(F)c1F ; Cc1ccc([C@H]2c3[nH]c4ccc(Cl)cc4c3CCN2c2nc(C)c(F)c(C(F)(F)F)n2)cc1]

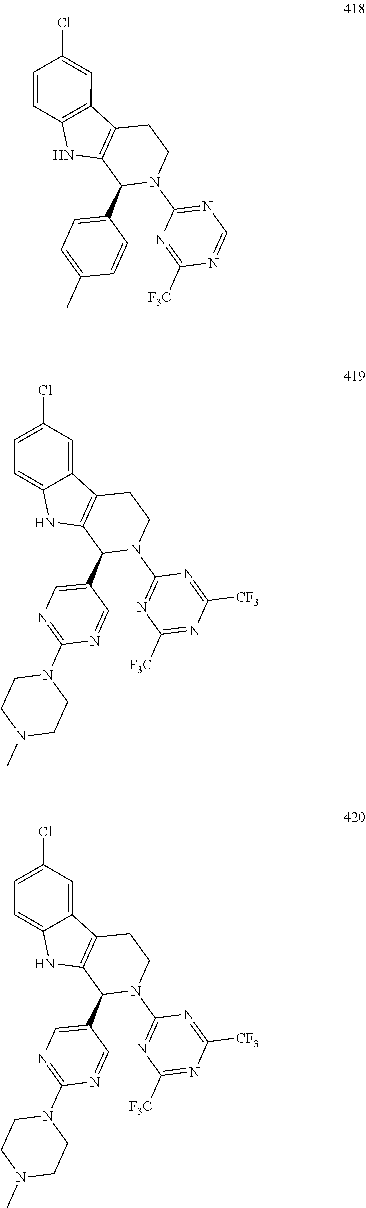 [SMILES: Cc1ccc([C@H]2c3[nH]c4ccc(Cl)cc4c3CCN2c2ncnc(C(F)(F)F)n2)cc1 ; CN1CCN(c2ncc([C@H]3c4[nH]c5ccc(Cl)cc5c4CCN3c3nc(C(F)(F)F)nc(C(F)(F)F)n3)cn2)CC1 ; CN1CCN(c2ncc([C@H]3c4[nH]c5ccc(Cl)cc5c4CCN3c3nc(C(F)(F)F)nc(C(F)(F)F)n3)cn2)CC1]

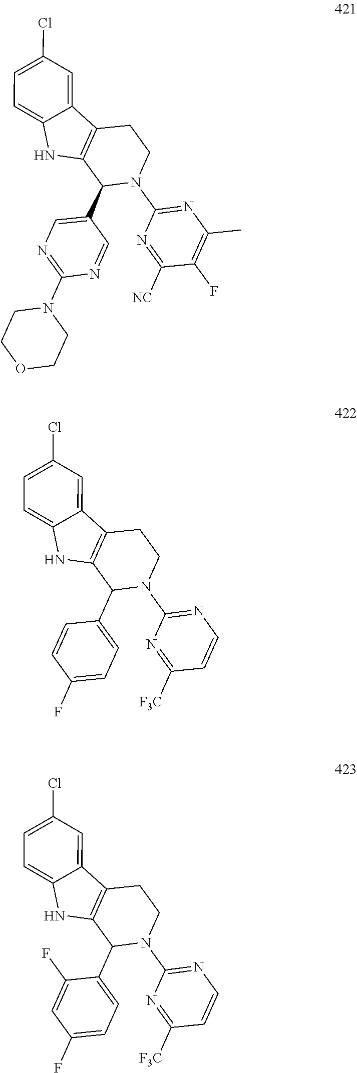 [SMILES: Cc1nc(N2CCc3c([nH]c4ccc(Cl)cc34)[C@@H]2c2cnc(N3CCOCC3)nc2)nc(C#N)c1F ; Fc1ccc(C2c3[nH]c4ccc(Cl)cc4c3CCN2c2nccc(C(F)(F)F)n2)cc1 ; Fc1ccc(C2c3[nH]c4ccc(Cl)cc4c3CCN2c2nccc(C(F)(F)F)n2)c(F)c1]

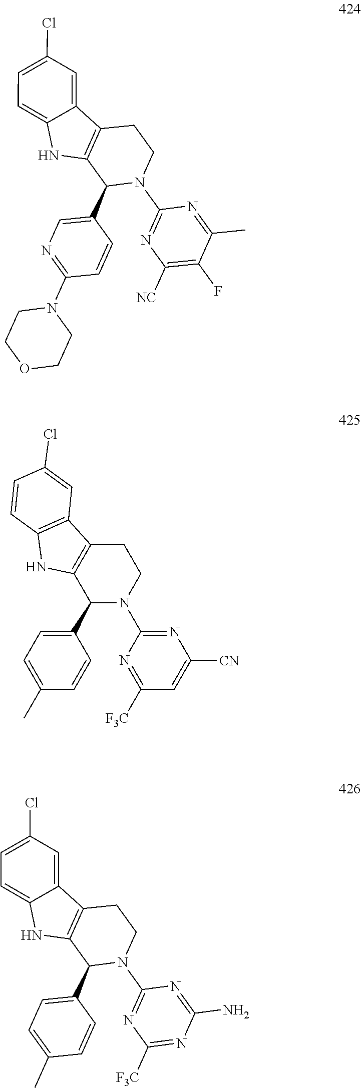 [SMILES: Cc1nc(N2CCc3c([nH]c4ccc(Cl)cc34)[C@@H]2c2ccc(N3CCOCC3)nc2)nc(C#N)c1F ; Cc1ccc([C@H]2c3[nH]c4ccc(Cl)cc4c3CCN2c2nc(C#N)cc(C(F)(F)F)n2)cc1 ; Cc1ccc([C@H]2c3[nH]c4ccc(Cl)cc4c3CCN2c2nc(N)nc(C(F)(F)F)n2)cc1]

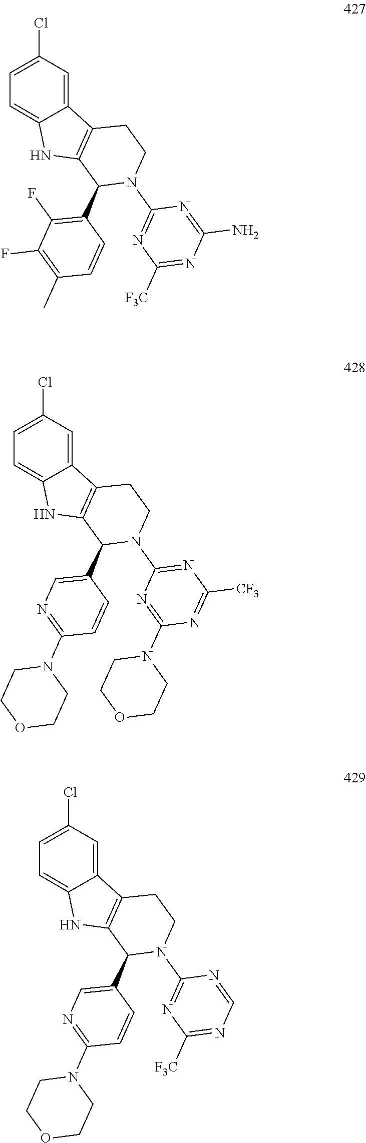 [SMILES: Cc1ccc([C@H]2c3[nH]c4ccc(Cl)cc4c3CCN2c2nc(N)nc(C(F)(F)F)n2)c(F)c1F ; FC(F)(F)c1nc(N2CCOCC2)nc(N2CCc3c([nH]c4ccc(Cl)cc34)[C@@H]2c2ccc(N3CCOCC3)nc2)n1 ; FC(F)(F)c1ncnc(N2CCc3c([nH]c4ccc(Cl)cc34)[C@@H]2c2ccc(N3CCOCC3)nc2)n1]

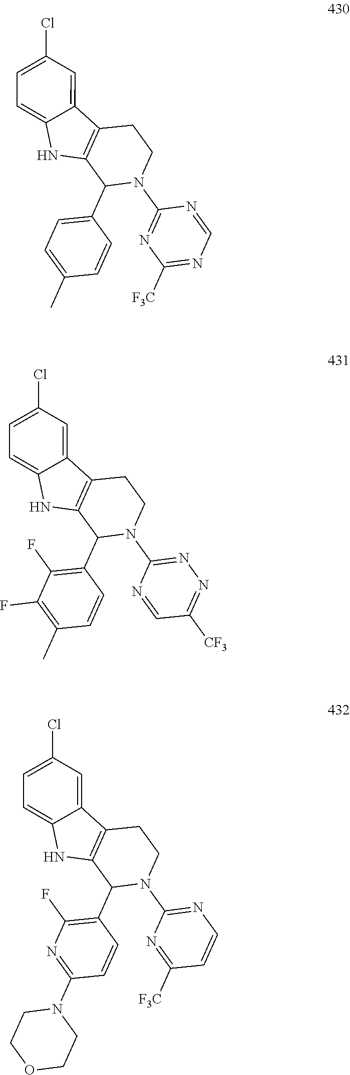 [SMILES: Cc1ccc(C2c3[nH]c4ccc(Cl)cc4c3CCN2c2ncnc(C(F)(F)F)n2)cc1 ; Cc1ccc(C2c3[nH]c4ccc(Cl)cc4c3CCN2c2ncc(C(F)(F)F)nn2)c(F)c1F ; Fc1nc(N2CCOCC2)ccc1C1c2[nH]c3ccc(Cl)cc3c2CCN1c1nccc(C(F)(F)F)n1]

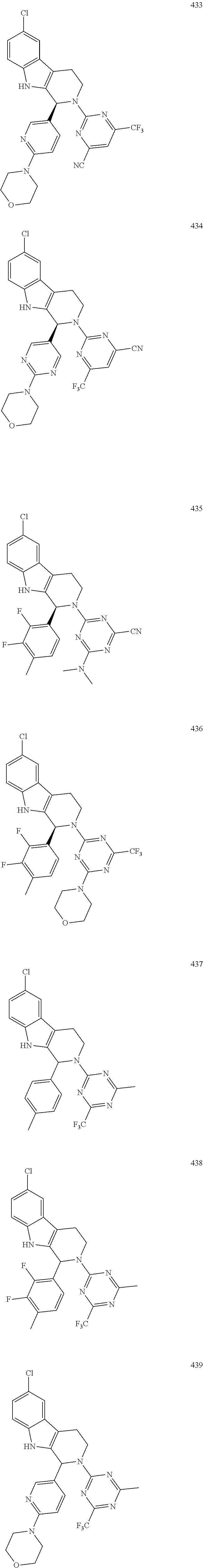

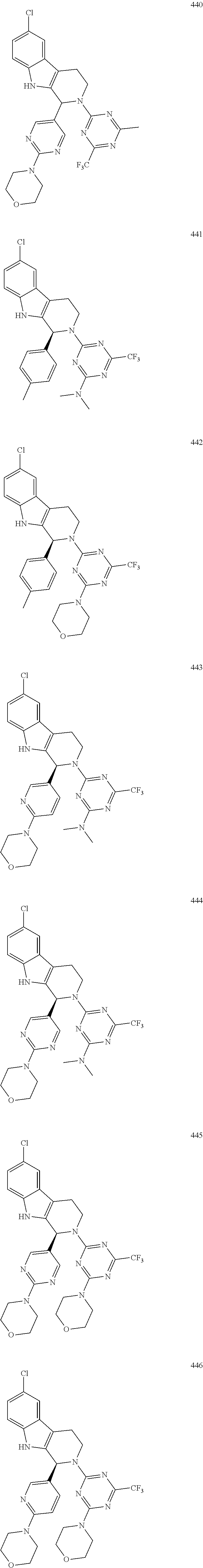

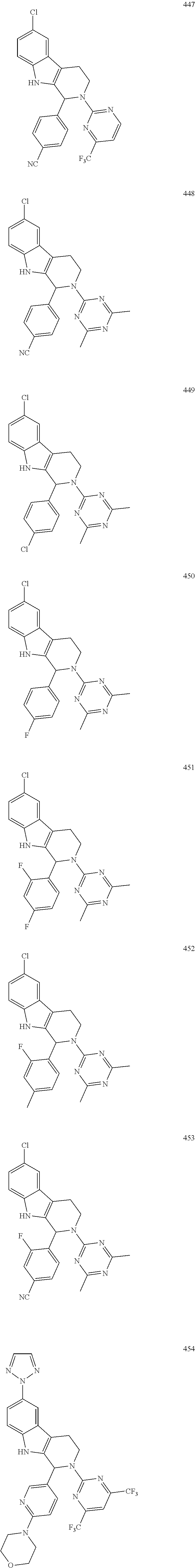

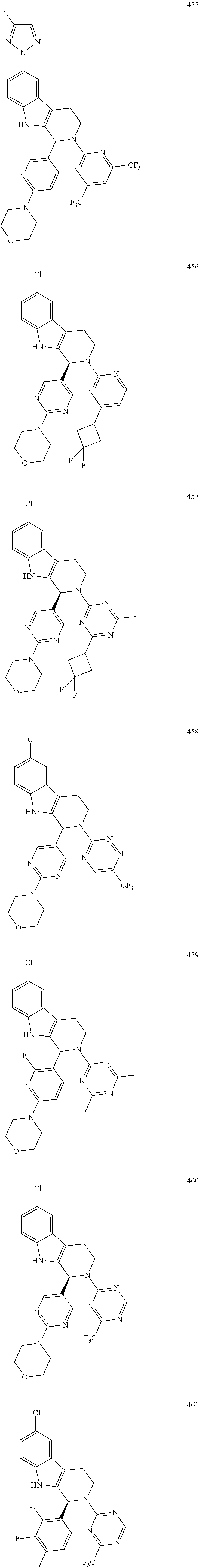

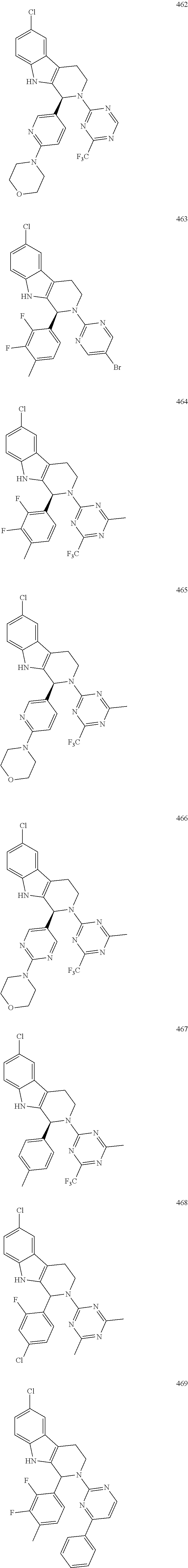

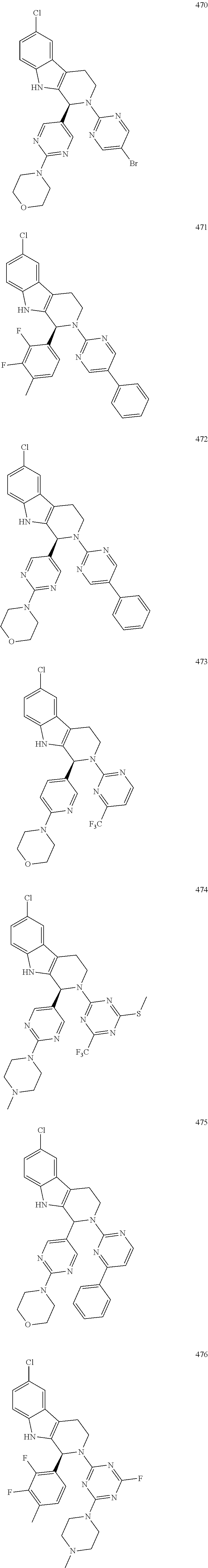

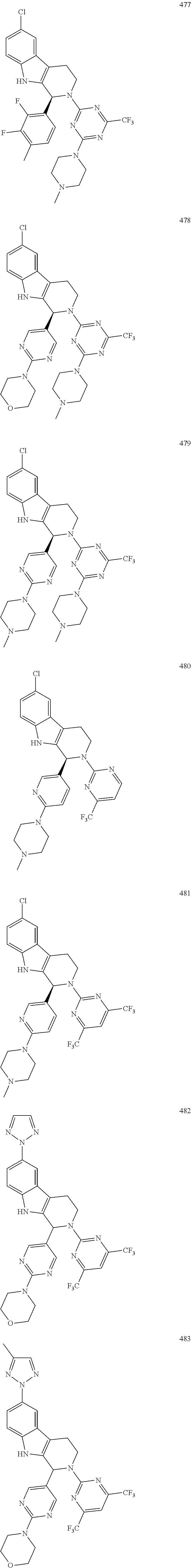

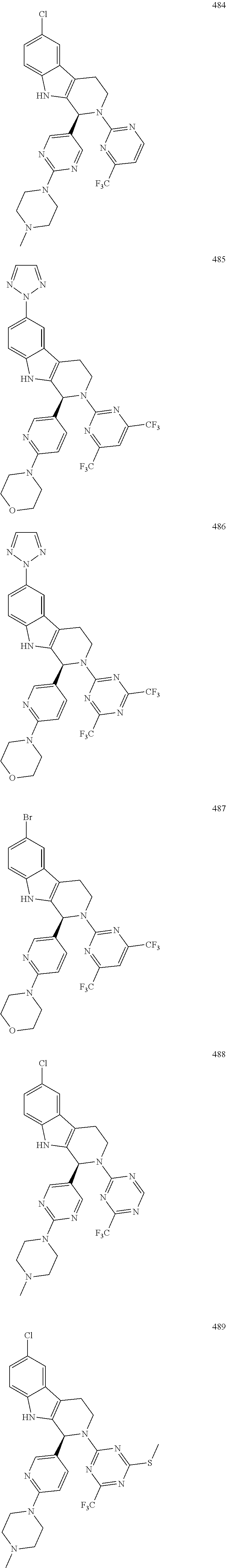

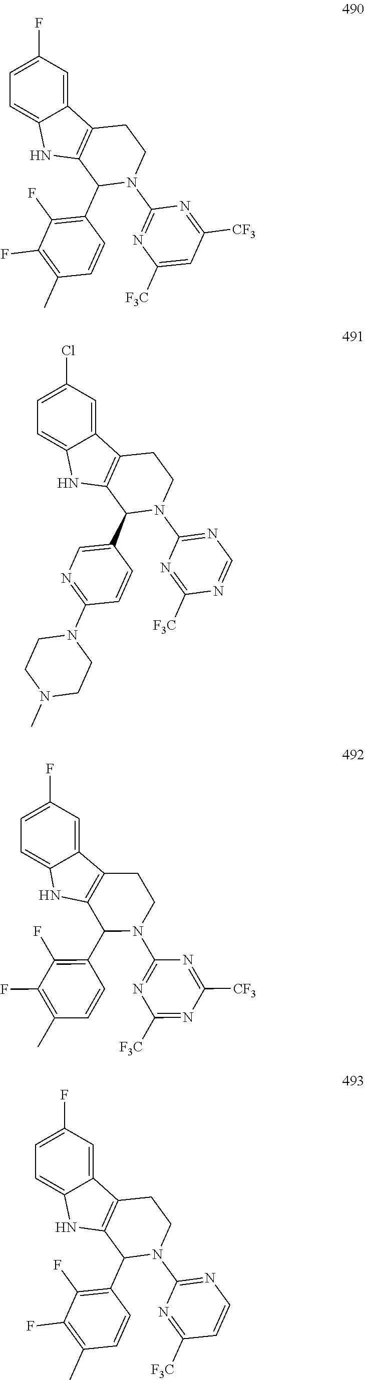

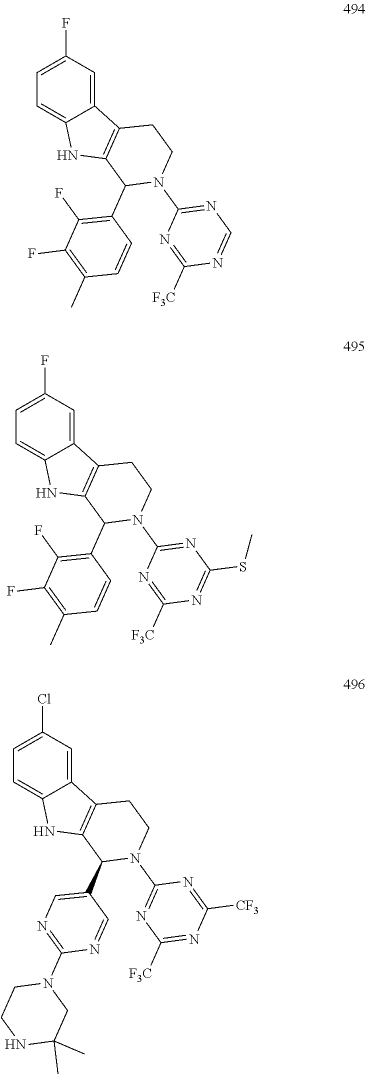 [SMILES: Cc1ccc(C2c3[nH]c4ccc(F)cc4c3CCN2c2ncnc(C(F)(F)F)n2)c(F)c1F ; CSc1nc(N2CCc3c([nH]c4ccc(F)cc34)C2c2ccc(C)c(F)c2F)nc(C(F)(F)F)n1 ; CC1(C)CN(c2ncc([C@H]3c4[nH]c5ccc(Cl)cc5c4CCN3c3nc(C(F)(F)F)nc(C(F)(F)F)n3)cn2)CCN1]

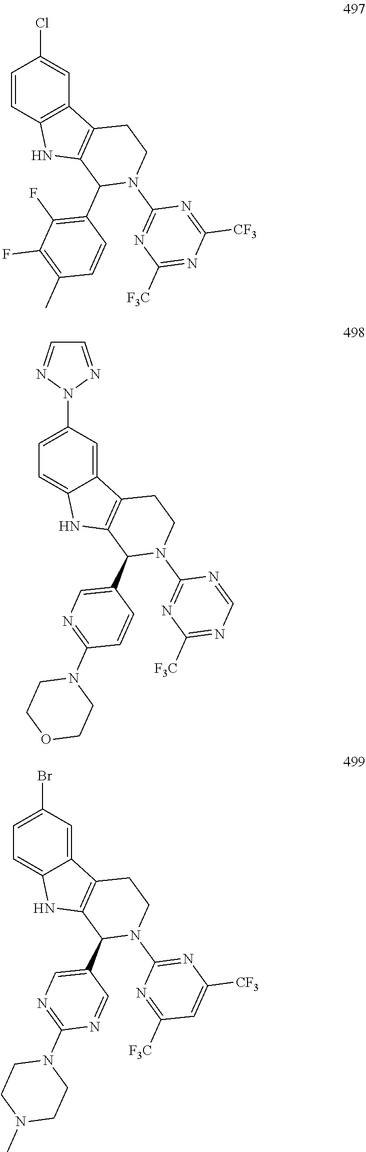

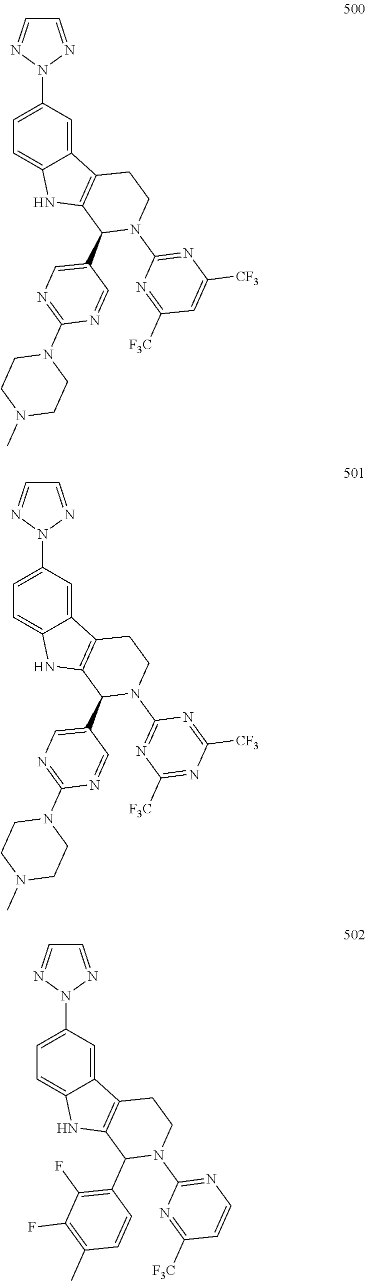

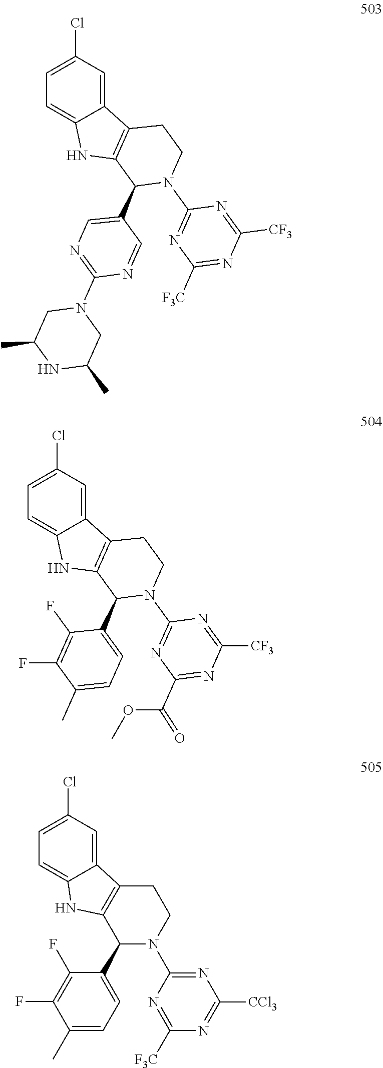

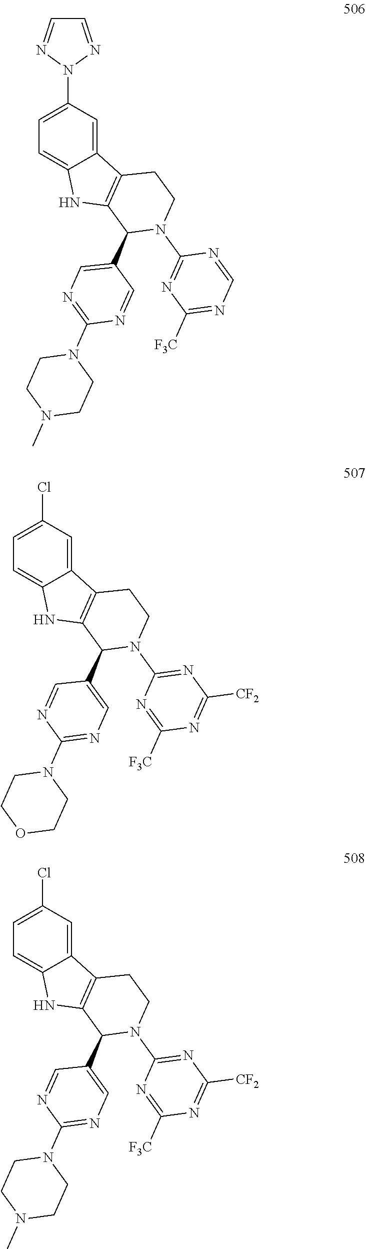

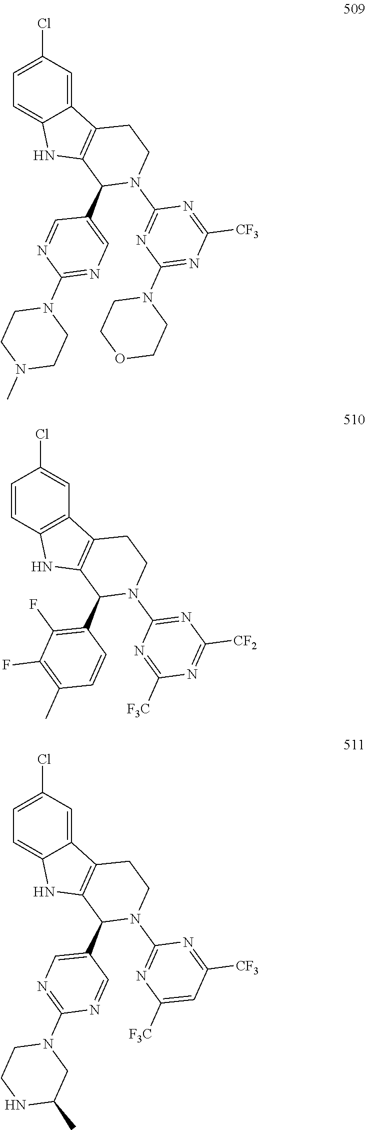

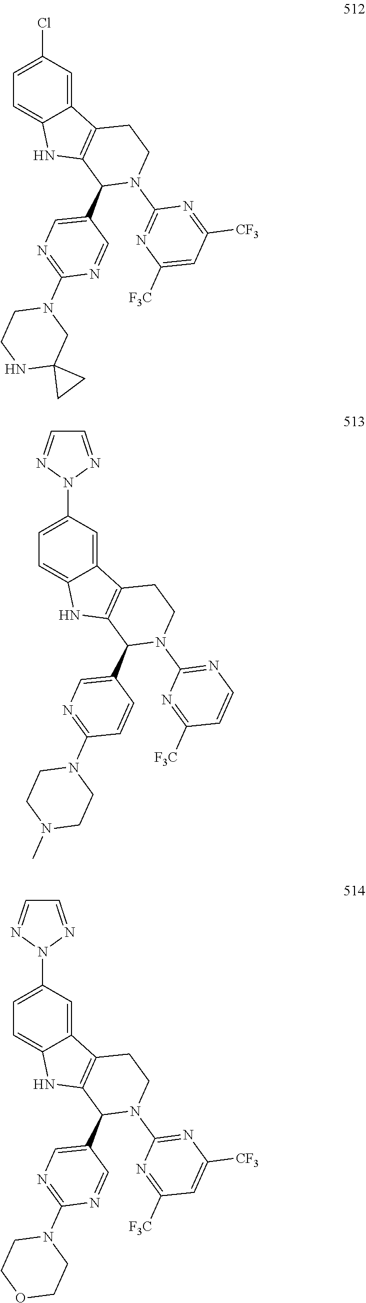

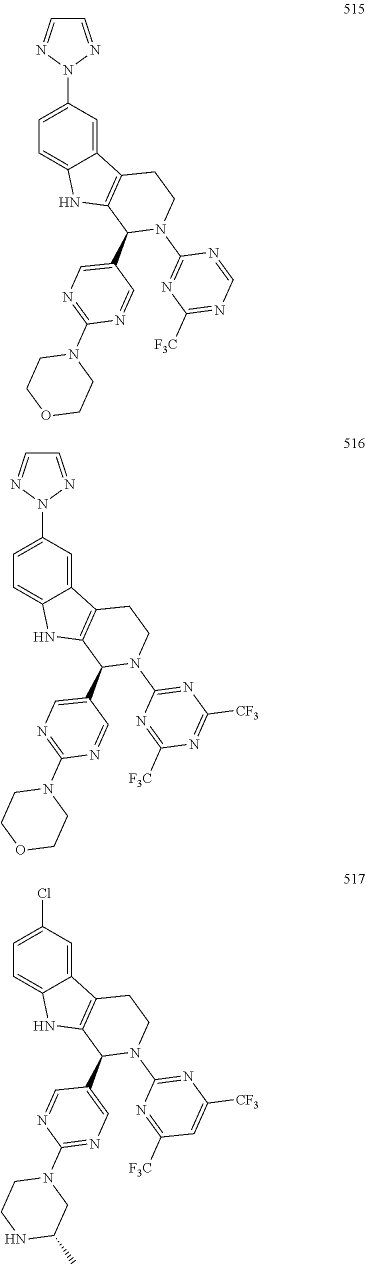

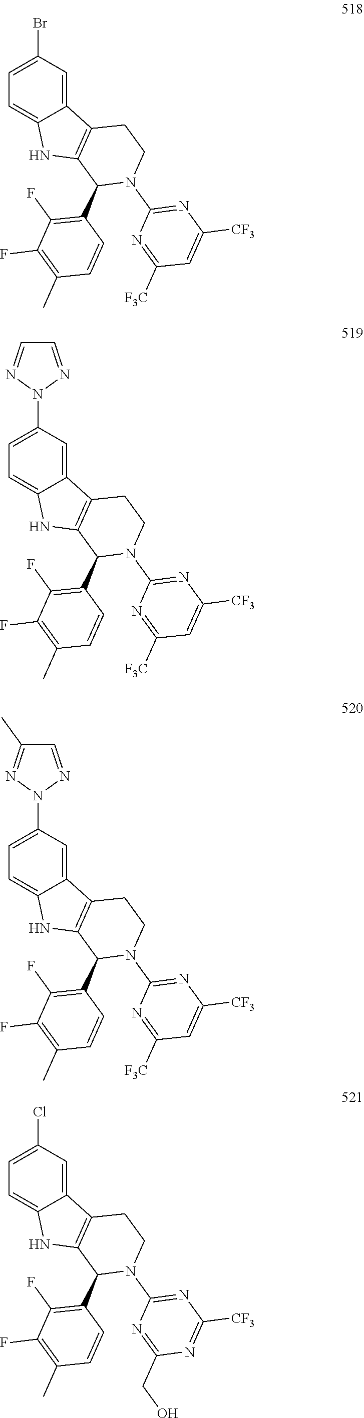

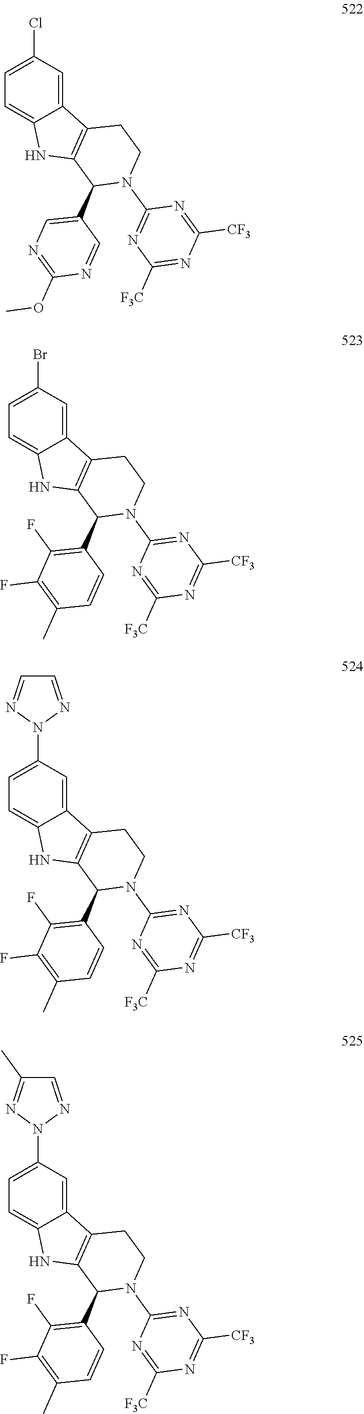

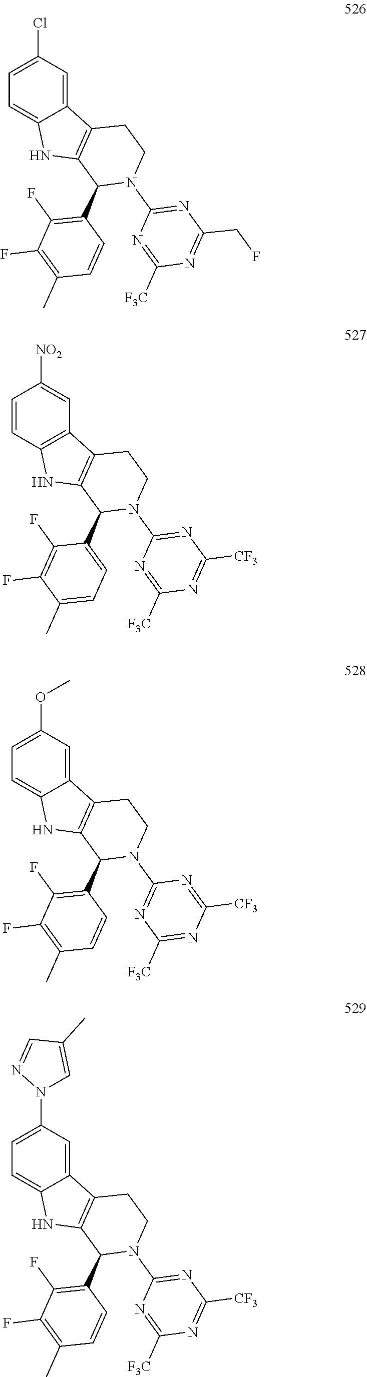

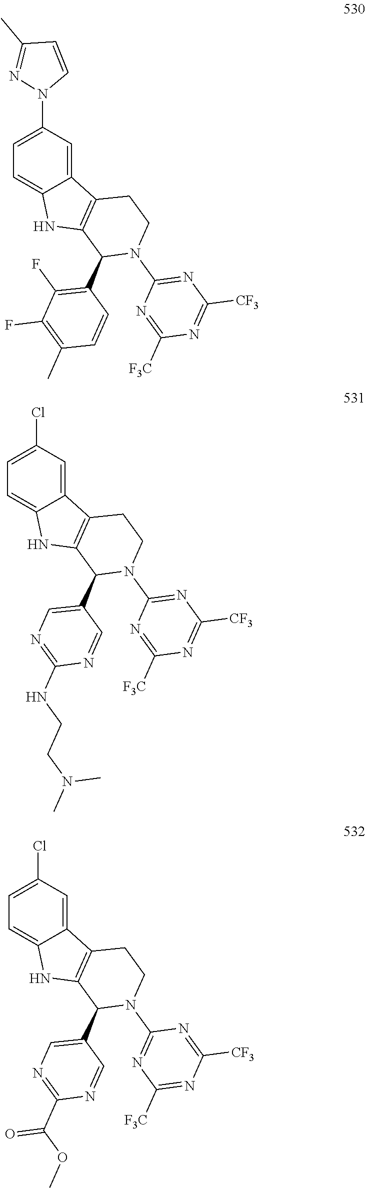

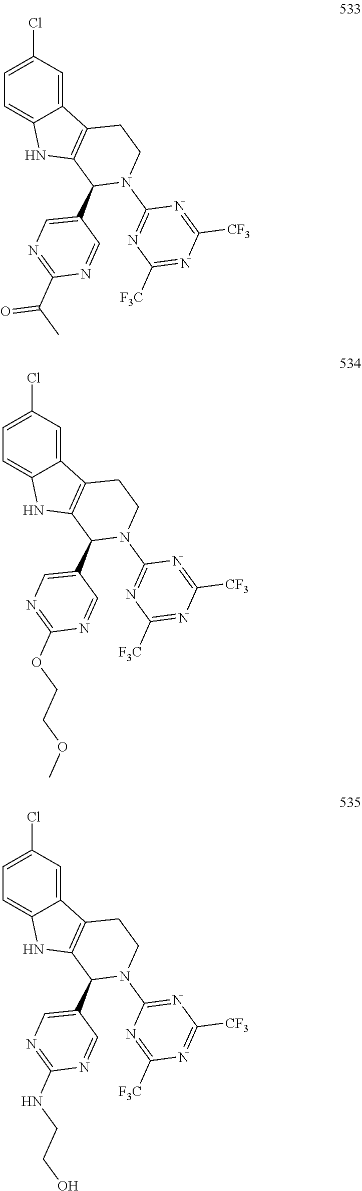

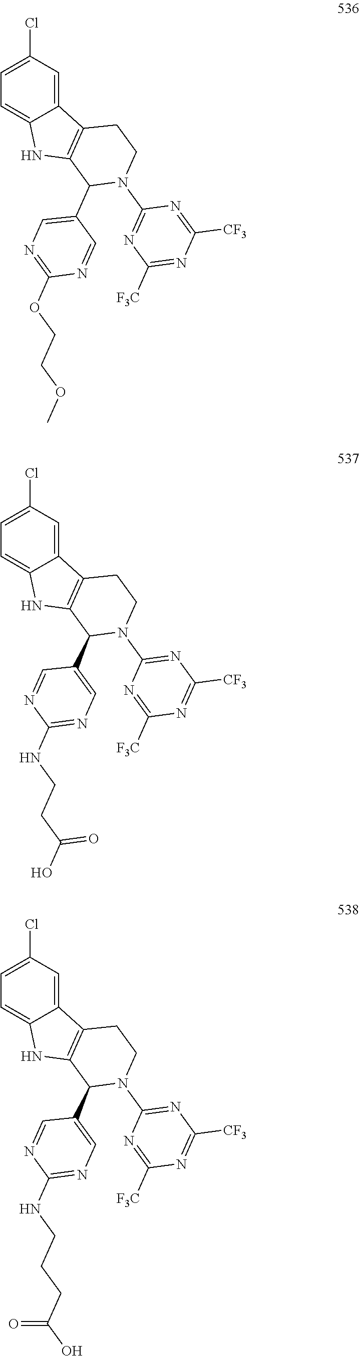 [SMILES: COCCOc1ncc(C2c3[nH]c4ccc(Cl)cc4c3CCN2c2nc(C(F)(F)F)nc(C(F)(F)F)n2)cn1 ; O=C(O)CCNc1ncc([C@H]2c3[nH]c4ccc(Cl)cc4c3CCN2c2nc(C(F)(F)F)nc(C(F)(F)F)n2)cn1 ; O=C(O)CCCNc1ncc([C@H]2c3[nH]c4ccc(Cl)cc4c3CCN2c2nc(C(F)(F)F)nc(C(F)(F)F)n2)cn1]

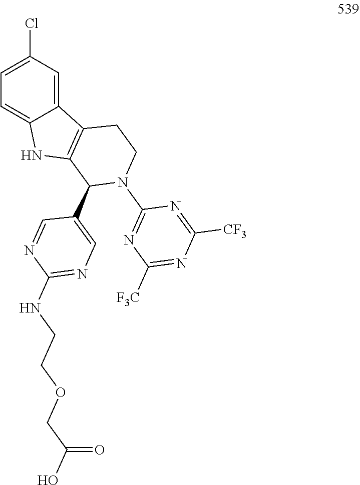 [SMILES: O=C(O)COCCNc1ncc([C@H]2c3[nH]c4ccc(Cl)cc4c3CCN2c2nc(C(F)(F)F)nc(C(F)(F)F)n2)cn1]

wherein the form of the compound is selected from the group consisting of a salt, hydrate, solvate, racemate, enantiomer, diastereomer, stereoisomer, and tautomer form thereof.

An aspect of the compound of Formula (I) or a form thereof includes a compound (Cpd) selected from the group consisting of (wherein ¹Cpd #indicates the compound was isolated as a salt form):

| Cpd | Name |
|---|---|
| 1 | 6-chloro-2-(5-chloropyrimidin-2-yl)-1-(4-fluorophenyl)-2,3,4,9-tetrahydro-1H- |
| | pyrido[3,4-b]indole; |
| 2 | 6-chloro-1-(4-fluorophenyl)-2-(5-methylpyrimidin-2-yl)-2,3,4,9-tetrahydro-1H- |
| | pyrido[3,4-b]indole; |
| 3 | 6-chloro-1-(4-fluorophenyl)-2-(5-fluoropyrimidin-2-yl)-2,3,4,9-tetrahydro-1H- |
| | pyrido[3,4-b]indole; |
| 4 | 6-chloro-2-(5-chloropyrimidin-2-yl)-7-fluoro-1-(4-fluorophenyl)-2,3,4,9-tetrahydro- |
| | 1H-pyrido[3,4-b]indole; |
| 5 | 6-chloro-7-fluoro-1-(4-fluorophenyl)-2-(5-fluoropyrimidin-2-yl)-2,3,4,9-tetrahydro- |
| | 1H-pyrido[3,4-b]indole; |
| 6 | 6-chloro-7-fluoro-2-(5-fluoropyrimidin-2-yl)-1-(4-methylphenyl)-2,3,4,9-tetrahydro- |
| | 1H-pyrido[3,4-b]indole; |
| 7 | 6-chloro-2-(4,6-dichloropyrimidin-2-yl)-1-(4-methylphenyl)-2,3,4,9-tetrahydro-1H- |
| | pyrido[3,4-b]indole; |
| 8 | 6-chloro-7-fluoro-2-(5-fluoropyrimidin-2-yl)-1-(6-methoxypyridin-3-yl)-2,3,4,9- |
| | tetrahydro-1H-pyrido[3,4-b]indole; |
| 9 | 6-chloro-7-fluoro-1-(4-fluorophenyl)-2-(4-methylpyrimidin-2-yl)-2,3,4,9-tetrahydro- |
| | 1H-pyrido[3,4-b]indole; |
| 10 | 6-chloro-2-(4-chloropyrimidin-2-yl)-1-(4-methylphenyl)-2,3,4,9-tetrahydro-1H- |
| | pyrido[3,4-b]indole; |
| 11 | 6-chloro-1-(4-chlorophenyl)-7-fluoro-2-(5-fluoropyrimidin-2-yl)-2,3,4,9-tetrahydro- |
| | 1H-pyrido[3,4-b]indole; |
| 12 | 6-chloro-1-(2,4-difluorophenyl)-7-fluoro-2-(5-fluoropyrimidin-2-yl)-2,3,4,9- |
| | tetrahydro-1H-pyrido[3,4-b]indole |
| 13 | 6-chloro-2-(4-chloropyrimidin-2-yl)-1-(4-fluorophenyl)-2,3,4,9-tetrahydro-1H- |
| | pyrido[3,4-b]indole; |
| 14 | 5-[6-chloro-7-fluoro-2-(5-fluoropyrimidin-2-yl)-2,3,4,9-tetrahydro-1H-pyrido[3,4- |
| | b]indol-1-yl]-N,N-dimethylpyrimidin-2-amine; |
| 15 | 6-chloro-2-(4-chloropyrimidin-2-yl)-7-fluoro-1-(4-fluorophenyl)-2,3,4,9-tetrahydro- |
| | 1H-pyrido[3,4-b]indole; |
| 16 | 6-chloro-2-(4,6-dichloropyrimidin-2-yl)-7-fluoro-1-(4-methylphenyl)-2,3,4,9- |
| | tetrahydro-1H-pyrido[3,4-b]indole; |
| 17 | 6-chloro-2-[6-chloro-7-fluoro-1-(4-methylphenyl)-1,3,4,9-tetrahydro-2H-pyrido[3,4- |
| | b]indol-2-yl]-N,N-dimethylpyrimidin-4-amine; |
| 18 | 4-[6-chloro-2-(4-chloropyrimidin-2-yl)-7-fluoro-2,3,4,9-tetrahydro-1H-pyrido[3,4- |
| | b]indol-1-yl]benzonitrile; |
| 19 | 6-chloro-2-(4,6-dimethylpyrimidin-2-yl)-7-fluoro-1-(4-fluorophenyl)-2,3,4,9- |
| | tetrahydro-1H-pyrido[3,4-b]indole; |
| 20 | 6-chloro-5-fluoro-2-(5-fluoropyrimidin-2-yl)-1-(4-methylphenyl)-2,3,4,9-tetrahydro- |
| | 1H-pyrido[3,4-b]indole; |
| 21 | 6-chloro-2-(5-chloropyrimidin-2-yl)-5-fluoro-1-(4-methylphenyl)-2,3,4,9-tetrahydro- |
| | 1H-pyrido[3,4-b]indole; |
| 22 | 6-chloro-2-(4-ethylpyrimidin-2-yl)-7-fluoro-1-(4-fluorophenyl)-2,3,4,9-tetrahydro- |
| | 1H-pyrido[3,4-b]indole; |
| 23 | 6-chloro-7-fluoro-1-(4-fluorophenyl)-2-[4-(propan-2-yl)pyrimidin-2-yl]-2,3,4,9- |
| | tetrahydro-1H-pyrido[3,4-b]indole; |
| 24 | 6-chloro-7-fluoro-1-(4-fluorophenyl)-2-[4-(trifluoromethyl)pyrimidin-2-yl]-2,3,4,9- |
| | tetrahydro-1H-pyrido[3,4-b]indole; |
| 25 | 2-[6-chloro-7-fluoro-1-(4-fluorophenyl)-1,3,4,9-tetrahydro-2H-pyrido[3,4-b]indol-2- |
| | yl]pyrimidine-4-carbonitrile; |
| 26 | 6-chloro-1-(4-chlorophenyl)-5-fluoro-2-(5-fluoropyrimidin-2-yl)-2,3,4,9-tetrahydro- |
| | 1H-pyrido[3,4-b]indole; |
| 27 | 6-chloro-1-(4-chlorophenyl)-2-(5-chloropyrimidin-2-yl)-5-fluoro-2,3,4,9-tetrahydro- |
| | 1H-pyrido[3,4-b]indole; |
| 28 | 6-chloro-2-(4-chloropyrimidin-2-yl)-5-fluoro-1-(4-methylphenyl)-2,3,4,9-tetrahydro- |
| | 1H-pyrido[3,4-b]indole; |
| 29 | 6-chloro-1-(4-chlorophenyl)-2-(5-chloropyrimidin-2-yl)-7-fluoro-2,3,4,9-tetrahydro- |
| | 1H-pyrido[3,4-b]indole; |
| 30 | 6-chloro-7-fluoro-1-(2-fluorophenyl)-2-(5-fluoropyrimidin-2-yl)-2,3,4,9-tetrahydro- |
| | 1H-pyrido[3,4-b]indole; |
| 31 | 6-chloro-1-(4-cyclopropylphenyl)-7-fluoro-2-(5-fluoropyrimidin-2-yl)-2,3,4,9- |
| | tetrahydro-1H-pyrido[3,4-b]indole; |
| 32 | 4-[6-chloro-7-fluoro-2-(5-fluoropyrimidin-2-yl)-2,3,4,9-tetrahydro-1H-pyrido[3,4- |
| | b]indol-1-yl]benzonitrile; |
| 33 | 4-[6-chloro-8-fluoro-2-(5-fluoropyrimidin-2-yl)-2,3,4,9-tetrahydro-1H-pyrido[3,4- |
| | b]indol-1-yl]benzonitrile; |
| 34 | 5-[6-chloro-7-fluoro-2-(5-fluoropyrimidin-2-yl)-2,3,4,9-tetrahydro-1H-pyrido[3,4- |
| | b]indol-1-yl]-N,N-dimethylpyridin-2-amine; |
| 35 | 6-chloro-7-fluoro-2-(5-fluoropyrimidin-2-yl)-1-[4-(trifluoromethyl)phenyl]-2,3,4,9- |
| | tetrahydro-1H-pyrido[3,4-b]indole; |
| 36 | 6-chloro-1-(2,3-difluorophenyl)-7-fluoro-2-(5-fluoropyrimidin-2-yl)-2,3,4,9- |
| | tetrahydro-1H-pyrido[3,4-b]indole; |
| 37 | 6-chloro-1-(3,4-difluorophenyl)-7-fluoro-2-(5-fluoropyrimidin-2-yl)-2,3,4,9- |
| | tetrahydro-1H-pyrido[3,4-b]indole; |
| 38 | 6-chloro-1-(4-chlorophenyl)-7-fluoro-2-(4-methylpyrimidin-2-yl)-2,3,4,9-tetrahydro- |
| | 1H-pyrido[3,4-b]indole; |
| 39 | 6-chloro-1-(4-chlorophenyl)-2-(4-chloropyrimidin-2-yl)-7-fluoro-2,3,4,9-tetrahydro- |
| | 1H-pyrido[3,4-b]indole; |
| 40 | 2-[6-chloro-1-(4-chlorophenyl)-7-fluoro-1,3,4,9-tetrahydro-2H-pyrido[3,4-b]indol-2- |
| | yl]pyrimidine-4-carbonitrile; |
| 41 | 6-chloro-2-(5-chloropyrimidin-2-yl)-7-fluoro-1-(4-methylphenyl)-2,3,4,9-tetrahydro- |
| | 1H-pyrido[3,4-b]indole; |
| 42 | 6-chloro-7-fluoro-1-(4-methylphenyl)-2-(4-methylpyrimidin-2-yl)-2,3,4,9-tetrahydro- |
| | 1H-pyrido[3,4-b]indole; |
| 43 | 6-chloro-2-(4-chloropyrimidin-2-yl)-7-fluoro-1-(4-methylphenyl)-2,3,4,9-tetrahydro- |
| | 1H-pyrido[3,4-b]indole; |
| 44 | 2-[6-chloro-7-fluoro-1-(4-methylphenyl)-1,3,4,9-tetrahydro-2H-pyrido[3,4-b]indol-2- |
| | yl]pyrimidine-4-carbonitrile; |
| 45 | 6-chloro-1-(4-cyclopropylphenyl)-7-fluoro-2-(pyrimidin-2-yl)-2,3,4,9-tetrahydro-1H- |
| | pyrido[3,4-b]indole; |
| 46 | 6-chloro-2-(4-chloro-6-methylpyrimidin-2-yl)-7-fluoro-1-(4-fluorophenyl)-2,3,4,9- |
| | tetrahydro-1H-pyrido[3,4-b]indole; |
| 47 | 6-chloro-7-fluoro-1-(6-fluoropyridin-3-yl)-2-(5-fluoropyrimidin-2-yl)-2,3,4,9- |
| | tetrahydro-1H-pyrido[3,4-b]indole; |
| 48 | 5-[6-chloro-7-fluoro-2-(5-fluoropyrimidin-2-yl)-2,3,4,9-tetrahydro-1H-pyrido[3,4- |
| | b]indol-1-yl]pyridin-2-ol; |
| 49 | 6-chloro-2-(5-chloropyrimidin-2-yl)-5-fluoro-1-(4-fluorophenyl)-2,3,4,9-tetrahydro- |
| | 1H-pyrido[3,4-b]indole; |
| 50 | 6-chloro-7-fluoro-2-(5-fluoropyrimidin-2-yl)-1-[4-(propan-2-yl)phenyl]-2,3,4,9- |
| | tetrahydro-1H-pyrido[3,4-b]indole; |
| 51 | 6-chloro-5-fluoro-1-(4-fluorophenyl)-2-(5-fluoropyrimidin-2-yl)-2,3,4,9-tetrahydro- |
| | 1H-pyrido[3,4-b]indole; |
| 52 | 4-[6-chloro-2-(5-chloropyrimidin-2-yl)-5-fluoro-2,3,4,9-tetrahydro-1H-pyrido[3,4- |
| | b]indol-1-yl]benzonitrile; |
| 53 | 6-chloro-2-(4-chloropyrimidin-2-yl)-7-fluoro-1-[4-(propan-2-yl)phenyl]-2,3,4,9- |
| | tetrahydro-1H-pyrido[3,4-b]indole; |
| 54 | 6-chloro-7-fluoro-2-(4-methylpyrimidin-2-yl)-1-[4-(propan-2-yl)phenyl]-2,3,4,9- |
| | tetrahydro-1H-pyrido[3,4-b]indole; |
| 55 | 2-{6-chloro-7-fluoro-1-[4-(propan-2-yl)phenyl]-1,3,4,9-tetrahydro-2H-pyrido[3,4- |
| | b]indol-2-yl}pyrimidine-4-carbonitrile; |
| 56 | 4-[6-chloro-5-fluoro-2-(5-fluoropyrimidin-2-yl)-2,3,4,9-tetrahydro-1H-pyrido[3,4- |
| | b]indol-1-yl]benzonitrile; |
| 57 | 6-chloro-2-(5-chloropyrimidin-2-yl)-7-fluoro-1-[4-(propan-2-yl)phenyl]-2,3,4,9- |
| | tetrahydro-1H-pyrido[3,4-b]indole; |
| 58 | 6-chloro-7-fluoro-1-(3-fluorophenyl)-2-(5-fluoropyrimidin-2-yl)-2,3,4,9-tetrahydro- |
| | 1H-pyrido[3,4-b]indole; |
| 59 | 4-[6-chloro-2-(5-chloropyrimidin-2-yl)-7-fluoro-2,3,4,9-tetrahydro-1H-pyrido[3,4- |
| | b]indol-1-yl]benzonitrile; |
| 60 | 6-chloro-5-fluoro-1-(4-fluorophenyl)-2-[4-(trifluoromethyl)pyrimidin-2-yl]-2,3,4,9- |
| | tetrahydro-1H-pyrido[3,4-b]indole; |
| 61 | 4-[6-chloro-7-fluoro-2-(4-methylpyrimidin-2-yl)-2,3,4,9-tetrahydro-1H-pyrido[3,4- |
| | b]indol-1-yl]benzonitrile; |
| 62 | 6-chloro-2-(5-chloropyrimidin-2-yl)-5-fluoro-1-[4-(trifluoromethyl)phenyl]-2,3,4,9- |
| | tetrahydro-1H-pyrido[3,4-b]indole; |
| 63 | 6-chloro-1-(2,3-difluorophenyl)-7-fluoro-2-(4-methylpyrimidin-2-yl)-2,3,4,9- |
| | tetrahydro-1H-pyrido[3,4-b]indole; |
| 64 | 2-[6-chloro-1-(4-cyanophenyl)-7-fluoro-1,3,4,9-tetrahydro-2H-pyrido[3,4-b]indol-2- |
| | yl]pyrimidine-4-carbonitrile; |
| 65 | {4-[6-chloro-2-(5-fluoropyrimidin-2-yl)-2,3,4,9-tetrahydro-1H-pyrido[3,4-b]indol-1- |
| | yl]phenyl}methanol; |
| 66 | 6-chloro-2-(4,6-dimethylpyrimidin-2-yl)-7-fluoro-1-(4-methylphenyl)-2,3,4,9- |
| | tetrahydro-1H-pyrido[3,4-b]indole; |
| 67 | 6-chloro-5-fluoro-2-(5-fluoropyrimidin-2-yl)-1-[4-(trifluoromethyl)phenyl]-2,3,4,9- |
| | tetrahydro-1H-pyrido[3,4-b]indole; |
| 68 | 2-[6-chloro-1-(2,3-difluorophenyl)-7-fluoro-1,3,4,9-tetrahydro-2H-pyrido[3,4-b]indol- |
| | 2-yl]pyrimidine-4-carbonitrile; |
| 69 | 4-[2-(4-bromopyrimidin-2-yl)-6-chloro-7-fluoro-2,3,4,9-tetrahydro-1H-pyrido[3,4- |
| | b]indol-1-yl]benzonitrile; |
| 70 | 6-chloro-1-(4-chlorophenyl)-5-fluoro-2-(5-fluoro-4-methylpyrimidin-2-yl)-2,3,4,9- |
| | tetrahydro-1H-pyrido[3,4-b]indole; |
| 71 | 6-chloro-1-(4-ethenylphenyl)-2-(5-fluoropyrimidin-2-yl)-2,3,4,9-tetrahydro-1H- |
| | pyrido[3,4-b]indole; |
| 72 | 6-chloro-7-fluoro-1-(4-methylphenyl)-2-[4-(trifluoromethyl)pyrimidin-2-yl]-2,3,4,9- |
| | tetrahydro-1H-pyrido[3,4-b]indole; |
| 73 | 6-chloro-1-(4-chlorophenyl)-5-fluoro-2-[4-(trifluoromethyl)pyrimidin-2-yl]-2,3,4,9- |
| | tetrahydro-1H-pyrido[3,4-b]indole; |
| 74 | 6-chloro-5-fluoro-1-[4-(trifluoromethyl)phenyl]-2-[4-(trifluoromethyl)pyrimidin-2- |
| | yl]-2,3,4,9-tetrahydro-1H-pyrido[3,4-b]indole; |
| 75 | 6-chloro-7-fluoro-1-[4-(trifluoromethyl)phenyl]-2-[4-(trifluoromethyl)pyrimidin-2- |
| | yl]-2,3,4,9-tetrahydro-1H-pyrido[3,4-b]indole; |
| 76 | 6-chloro-5-fluoro-1-(4-methylphenyl)-2-[4-(trifluoromethyl)pyrimidin-2-yl]-2,3,4,9- |
| | tetrahydro-1H-pyrido[3,4-b]indole; |
| 77 | 6-chloro-2-(4-chloropyrimidin-2-yl)-1-(2,3-difluorophenyl)-7-fluoro-2,3,4,9- |
| | tetrahydro-1H-pyrido[3,4-b]indole; |
| 78 | {4-[6-chloro-7-fluoro-2-(5-fluoropyrimidin-2-yl)-2,3,4,9-tetrahydro-1H-pyrido[3,4- |
| | b]indol-1-yl]phenyl}methanol; |
| 79 | 6-chloro-1-(4-ethenylphenyl)-7-fluoro-2-(5-fluoropyrimidin-2-yl)-2,3,4,9-tetrahydro- |
| | 1H-pyrido[3,4-b]indole; |
| 80 | 6-chloro-2-(5-chloropyrimidin-2-yl)-7-fluoro-1-[4-(trifluoromethyl)phenyl]-2,3,4,9- |
| | tetrahydro-1H-pyrido[3,4-b]indole; |
| 81 | 6-chloro-7-fluoro-2-(4-methylpyrimidin-2-yl)-1-[4-(trifluoromethyl)phenyl]-2,3,4,9- |
| | tetrahydro-1H-pyrido[3,4-b]indole; |
| 82 | 5,6-difluoro-2-(5-fluoropyrimidin-2-yl)-1-(4-methylphenyl)-2,3,4,9-tetrahydro-1H- |
| | pyrido[3,4-b]indole; |
| 83 | 5,6-difluoro-1-(4-fluorophenyl)-2-(5-fluoropyrimidin-2-yl)-2,3,4,9-tetrahydro-1H- |
| | pyrido[3,4-b]indole; |
| 84 | 2-(5-chloropyrimidin-2-yl)-5,6-difluoro-1-(4-methylphenyl)-2,3,4,9-tetrahydro-1H- |
| | pyrido[3,4-b]indole; |
| 85 | 2-(5-chloropyrimidin-2-yl)-5,6-difluoro-1-(4-fluorophenyl)-2,3,4,9-tetrahydro-1H- |
| | pyrido[3,4-b]indole; |
| 86 | 2-{4-[6-chloro-7-fluoro-2-(5-fluoropyrimidin-2-yl)-2,3,4,9-tetrahydro-1H-pyrido[3,4- |
| | b]indol-1-yl]phenyl}ethan-1-ol; |
| 87 | methyl 2-[6-chloro-1-(4-chlorophenyl)-5-fluoro-1,3,4,9-tetrahydro-2H-pyrido[3,4- |
| | b]indol-2-yl]pyrimidine-4-carboxylate; |
| 88 | 4-{6-chloro-7-fluoro-2-[4-methyl-6-(trifluoromethyl)pyrimidin-2-yl]-2,3,4,9- |
| | tetrahydro-1H-pyrido[3,4-b]indol-1-yl}benzonitrile; |
| 89 | 4-{6-chloro-5-fluoro-2-[4-(trifluoromethyl)pyrimidin-2-yl]-2,3,4,9-tetrahydro-1H- |
| | pyrido[3,4-b]indol-1-yl}benzonitrile; |
| 90 | 6-chloro-5-fluoro-1-(4-fluorophenyl)-2-(4-methylpyrimidin-2-yl)-2,3,4,9-tetrahydro- |
| | 1H-pyrido[3,4-b]indole; |
| 91 | 6-chloro-7-fluoro-2-(5-fluoropyrimidin-2-yl)-1-(2-methylpyrimidin-5-yl)-2,3,4,9- |
| | tetrahydro-1H-pyrido[3,4-b]indole; |
| 92 | 6-chloro-7-fluoro-1-(2-methylpyrimidin-5-yl)-2-[4-(trifluoromethyl)pyrimidin-2-yl]- |
| | 2,3,4,9-tetrahydro-1H-pyrido[3,4-b]indole; |
| 93 | 4-[6-chloro-2-(4,6-dimethylpyrimidin-2-yl)-7-fluoro-2,3,4,9-tetrahydro-1H- |
| | pyrido[3,4-b]indol-1-yl]benzonitrile; |
| 94 | 5-[6-chloro-7-fluoro-2-(4-methylpyrimidin-2-yl)-2,3,4,9-tetrahydro-1H-pyrido[3,4- |
| | b]indol-1-yl]-N,N-dimethylpyrimidin-2-amine; |
| 95 | 5-{6-chloro-7-fluoro-2-[4-(trifluoromethyl)pyrimidin-2-yl]-2,3,4,9-tetrahydro-1H- |
| | pyrido[3,4-b]indol-1-yl}-N,N-dimethylpyrimidin-2-amine; |
| 96 | 6-chloro-7-fluoro-1-(4-methylphenyl)-2-[4-methyl-6-(trifluoromethyl)pyrimidin-2- |
| | yl]-2,3,4,9-tetrahydro-1H-pyrido[3,4-b]indole; |
| 97 | 6-chloro-7-fluoro-1-(4-methylphenyl)-2-[5-(trifluoromethyl)pyrimidin-2-yl]-2,3,4,9- |
| | tetrahydro-1H-pyrido[3,4-b]indole; |
| 98 | 6-chloro-7-fluoro-1-(4-fluorophenyl)-2-[5-(trifluoromethyl)pyrimidin-2-yl]-2,3,4,9- |
| | tetrahydro-1H-pyrido[3,4-b]indole; |
| 99 | 6-chloro-7-fluoro-1-[4-(propan-2-yl)phenyl]-2-[5-(trifluoromethyl)pyrimidin-2-yl]- |
| | 2,3,4,9-tetrahydro-1H-pyrido[3,4-b]indole; |
| 100 | 4-{6-chloro-7-fluoro-2-[4-(trifluoromethyl)pyrimidin-2-yl]-2,3,4,9-tetrahydro-1H- |
| | pyrido[3,4-b]indol-1-yl}benzonitrile; |
| 101 | 6-chloro-2-(5-chloropyrimidin-2-yl)-7-fluoro-1-(2-methylpyrimidin-5-yl)-2,3,4,9- |
| | tetrahydro-1H-pyrido[3,4-b]indole; |
| 102 | 5-[6-chloro-2-(5-chloropyrimidin-2-yl)-7-fluoro-2,3,4,9-tetrahydro-1H-pyrido[3,4- |
| | b]indol-1-yl]-N,N-dimethylpyrimidin-2-amine; |
| 103 | 6-chloro-7-fluoro-1-[4-(methanesulfonyl)phenyl]-2-[4-(trifluoromethyl)pyrimidin-2- |
| | yl]-2,3,4,9-tetrahydro-1H-pyrido[3,4-b]indole; |
| 104 | methyl 2-[6-chloro-1-(4-cyanophenyl)-5-fluoro-1,3,4,9-tetrahydro-2H-pyrido[3,4- |
| | b]indol-2-yl]pyrimidine-4-carboxylate; |
| 105 | 6-chloro-7-fluoro-2-(5-fluoro-4-methylpyrimidin-2-yl)-1-(4-fluorophenyl)-2,3,4,9- |
| | tetrahydro-1H-pyrido[3,4-b]indole; |
| 106 | 6-chloro-7-fluoro-2-(5-fluoro-4,6-dimethylpyrimidin-2-yl)-1-(4-methylphenyl)- |
| | 2,3,4,9-tetrahydro-1H-pyrido[3,4-b]indole; |
| 107 | 6-chloro-7-fluoro-2-(5-fluoropyrimidin-2-yl)-1-[4-(methanesulfonyl)phenyl]-2,3,4,9- |
| | tetrahydro-1H-pyrido[3,4-b]indole; |
| 108 | 6-chloro-1-(2,3-difluorophenyl)-7-fluoro-2-[4-(trifluoromethyl)pyrimidin-2-yl]- |
| | 2,3,4,9-tetrahydro-1H-pyrido[3,4-b]indole; |
| 109 | 6-chloro-5-fluoro-2-(5-fluoro-4-methylpyrimidin-2-yl)-1-(4-methylphenyl)-2,3,4,9- |
| | tetrahydro-1H-pyrido[3,4-b]indole; |
| 110 | 2-[6-chloro-5-fluoro-1-(4-methylphenyl)-1,3,4,9-tetrahydro-2H-pyrido[3,4-b]indol-2- |
| | yl]-6-methylpyrimidine-4-carbonitrile; |
| 111 | 2-[6-chloro-5-fluoro-1-(4-fluorophenyl)-1,3,4,9-tetrahydro-2H-pyrido[3,4-b]indol-2- |
| | yl]-6-methylpyrimidine-4-carbonitrile; |
| 112 | 6-chloro-1-(2,3-difluorophenyl)-7-fluoro-2-(5-fluoro-4,6-dimethylpyrimidin-2-yl)- |
| | 2,3,4,9-tetrahydro-1H-pyrido[3,4-b]indole; |
| 113 | 6-chloro-1-(2,3-difluorophenyl)-5-fluoro-2-(5-fluoro-4-methylpyrimidin-2-yl)-2,3,4,9- |
| | tetrahydro-1H-pyrido[3,4-b]indole; |
| 114 | 6-chloro-1-(2,3-difluoro-4-methylphenyl)-5-fluoro-2-(5-fluoro-4-methylpyrimidin-2- |
| | yl)-2,3,4,9-tetrahydro-1H-pyrido[3,4-b]indole |
| 115 | 4-{6-chloro-7-fluoro-2-[4-(trifluoromethyl)pyrimidin-2-yl]-2,3,4,9-tetrahydro-1H- |
| | pyrido[3,4-b]indol-1-yl}benzene-1-sulfonamide; |
| 116 | 6-chloro-5-fluoro-2-(5-fluoro-4,6-dimethylpyrimidin-2-yl)-1-(4-methylphenyl)- |
| | 2,3,4,9-tetrahydro-1H-pyrido[3,4-b]indole; |
| 117 | 6-chloro-1-(2,3-difluorophenyl)-5-fluoro-2-(5-fluoro-4,6-dimethylpyrimidin-2-yl)- |
| | 2,3,4,9-tetrahydro-1H-pyrido[3,4-b]indole; |
| 118 | 6-chloro-1-(2,3-difluoro-4-methylphenyl)-5-fluoro-2-(5-fluoro-4,6- |
| | dimethylpyrimidin-2-yl)-2,3,4,9-tetrahydro-1H-pyrido[3,4-b]indole; |
| 119 | 6-chloro-1-(2,3-difluorophenyl)-2-[4-(trifluoromethyl)pyrimidin-2-yl]-2,3,4,9- |
| | tetrahydro-1H-pyrido[3,4-b]indole; |
| 120 | 6-chloro-1-(2,3-difluoro-4-methoxyphenyl)-2-[4-(trifluoromethyl)pyrimidin-2-yl]- |
| | 2,3,4,9-tetrahydro-1H-pyrido[3,4-b]indole; |
| 121 | 2-{6-chloro-7-fluoro-1-[4-(trifluoromethyl)phenyl]-1,3,4,9-tetrahydro-2H-pyrido[3,4- |
| | b]indol-2-yl}pyrimidine-4-carbonitrile; |
| 122 | 5-{6-chloro-7-fluoro-2-[4-(trifluoromethyl)pyrimidin-2-yl]-2,3,4,9-tetrahydro-1H- |
| | pyrido[3,4-b]indol-1-yl}-N,N-dimethylpyridin-2-amine; |
| 123 | 6-chloro-7-fluoro-2-(5-fluoro-4-methylpyrimidin-2-yl)-1-(4-methylphenyl)-2,3,4,9- |
| | tetrahydro-1H-pyrido[3,4-b]indole; |
| 124 | 6-chloro-1-(2,3-difluorophenyl)-7-fluoro-2-(5-fluoro-4-methylpyrimidin-2-yl)-2,3,4,9- |
| | tetrahydro-1H-pyrido[3,4-b]indole; |
| 125 | 6-chloro-1-(2,3-difluoro-4-methylphenyl)-7-fluoro-2-(5-fluoro-4-methylpyrimidin-2- |
| | yl)-2,3,4,9-tetrahydro-1H-pyrido[3,4-b]indole; |
| 126 | 5-[6-chloro-7-fluoro-2-(4-methylpyrimidin-2-yl)-2,3,4,9-tetrahydro-1H-pyrido[3,4- |
| | b]indol-1-yl]-N,N-dimethylpyridin-2-amine; |
| 127 | 4-[6-chloro-7-fluoro-2-(5-fluoropyrimidin-2-yl)-2,3,4,9-tetrahydro-1H-pyrido[3,4- |
| | b]indol-1-yl]benzene-1-sulfonamide; |
| 128 | 6-chloro-1-(2,3-difluoro-4-methylphenyl)-2-[4-(trifluoromethyl)pyrimidin-2-yl]- |
| | 2,3,4,9-tetrahydro-1H-pyrido[3,4-b]indole; |
| 129 | 6-chloro-1-(2,3-difluorophenyl)-2-(4,6-dimethylpyrimidin-2-yl)-7-fluoro-2,3,4,9- |
| | tetrahydro-1H-pyrido[3,4-b]indole; |
| 130 | 6-chloro-2-(5-chloropyrimidin-2-yl)-1-(2,3-difluoro-4-methylphenyl)-7-fluoro- |
| | 2,3,4,9-tetrahydro-1H-pyrido[3,4-b]indole; |
| 131 | 6-chloro-2-(5-chloropyrimidin-2-yl)-1-(2,3-difluorophenyl)-7-fluoro-2,3,4,9- |
| | tetrahydro-1H-pyrido[3,4-b]indole; |
| 132 | 6-chloro-1-(2,3-difluoro-4-methylphenyl)-2-(4,6-dimethylpyrimidin-2-yl)-7-fluoro- |
| | 2,3,4,9-tetrahydro-1H-pyrido[3,4-b]indole; |
| 133 | 6-chloro-1-(2,3-difluorophenyl)-7-fluoro-2-[5-(trifluoromethyl)pyrimidin-2-yl]- |
| | 2,3,4,9-tetrahydro-1H-pyrido[3,4-b]indole; |
| 134 | 6-chloro-7-fluoro-1-(4-methylphenyl)-2-(5-methylpyrimidin-2-yl)-2,3,4,9-tetrahydro- |
| | 1H-pyrido[3,4-b]indole; |
| 135 | 6-chloro-5-fluoro-1-(4-methylphenyl)-2-(5-methylpyrimidin-2-yl)-2,3,4,9-tetrahydro- |
| | 1H-pyrido[3,4-b]indole; |
| 136 | 6-chloro-1-(2,3-difluoro-4-methylphenyl)-7-fluoro-2-(5-methylpyrimidin-2-yl)- |
| | 2,3,4,9-tetrahydro-1H-pyrido[3,4-b]indole; |
| 137 | 6-chloro-1-(2,3-difluoro-4-methylphenyl)-5-fluoro-2-(5-methylpyrimidin-2-yl)- |
| | 2,3,4,9-tetrahydro-1H-pyrido[3,4-b]indole; |
| 138 | 6-chloro-1-(2,3-difluoro-4-methylphenyl)-7-fluoro-2-(5-fluoropyrimidin-2-yl)-2,3,4,9- |
| | tetrahydro-1H-pyrido[3,4-b]indole; |
| 139 | 6-chloro-1-(2,3-difluoro-4-methylphenyl)-7-fluoro-2-(4-methylpyrimidin-2-yl)- |
| | 2,3,4,9-tetrahydro-1H-pyrido[3,4-b]indole; |
| 140 | 6-chloro-1-(2,3-difluoro-4-methylphenyl)-7-fluoro-2-[4-(trifluoromethyl)pyrimidin-2- |
| | yl]-2,3,4,9-tetrahydro-1H-pyrido[3,4-b]indole; |
| 141 | 2-[6-chloro-1-(2,3-difluoro-4-methylphenyl)-7-fluoro-1,3,4,9-tetrahydro-2H- |
| | pyrido[3,4-b]indol-2-yl]pyrimidine-4-carbonitrile; |
| 142 | 6-chloro-1-(2,3-difluoro-4-methylphenyl)-7-fluoro-2-[5-(trifluoromethyl)pyrimidin-2- |
| | yl]-2,3,4,9-tetrahydro-1H-pyrido[3,4-b]indole; |
| 143 | 6-chloro-1-(2,3-difluoro-4-methylphenyl)-7-fluoro-2-(5-fluoro-4,6- |
| | dimethylpyrimidin-2-yl)-2,3,4,9-tetrahydro-1H-pyrido[3,4-b]indole; |
| 144 | 6-chloro-1-(4-chloro-2,3-difluorophenyl)-2-[4-(trifluoromethyl)pyrimidin-2-yl]- |
| | 2,3,4,9-tetrahydro-1H-pyrido[3,4-b]indole; |
| 145 | 6-chloro-7-fluoro-1-(5-fluoropyridin-3-yl)-2-[4-(trifluoromethyl)pyrimidin-2-yl]- |
| | 2,3,4,9-tetrahydro-1H-pyrido[3,4-b]indole; |
| 146 | 6-chloro-5-fluoro-1-(5-fluoropyridin-3-yl)-2-[4-(trifluoromethyl)pyrimidin-2-yl]- |
| | 2,3,4,9-tetrahydro-1H-pyrido[3,4-b]indole; |
| 147 | 6-chloro-1-(2,3-difluorophenyl)-7-fluoro-2-[4-methyl-6-(trifluoromethyl)pyrimidin-2- |
| | yl]-2,3,4,9-tetrahydro-1H-pyrido[3,4-b]indole; |
| 148 | 6-chloro-1-(2,3-difluoro-4-methylphenyl)-7-fluoro-2-[4-methyl-6- |
| | (trifluoromethyl)pyrimidin-2-yl]-2,3,4,9-tetrahydro-1H-pyrido[3,4-b]indole; |
| 149 | 6-chloro-5-fluoro-1-[4-(methanesulfonyl)phenyl]-2-[4-(trifluoromethyl)pyrimidin-2- |
| | yl]-2,3,4,9-tetrahydro-1H-pyrido[3,4-b]indole; |
| 150 | 4-{6-chloro-5-fluoro-2-[4-(trifluoromethyl)pyrimidin-2-yl]-2,3,4,9-tetrahydro-1H- |
| | pyrido[3,4-b]indol-1-yl}benzene-1-sulfonamide; |
| 151 | 2-[6-chloro-7-fluoro-1-(4-methylphenyl)-1,3,4,9-tetrahydro-2H-pyrido[3,4-b]indol-2- |
| | yl]-6-methylpyrimidine-4-carbonitrile; |
| 152 | 2-[6-chloro-1-(2,3-difluorophenyl)-7-fluoro-1,3,4,9-tetrahydro-2H-pyrido[3,4-b]indol- |
| | 2-yl]-6-methylpyrimidine-4-carbonitrile; |
| 153 | 2-[6-chloro-1-(2,3-difluoro-4-methylphenyl)-7-fluoro-1,3,4,9-tetrahydro-2H- |
| | pyrido[3,4-b]indol-2-yl]-6-methylpyrimidine-4-carbonitrile; |
| 154 | 2-[4,6-bis(trifluoromethyl)pyrimidin-2-yl]-6-chloro-7-fluoro-1-(4-methylphenyl)- |
| | 2,3,4,9-tetrahydro-1H-pyrido[3,4-b]indole; |
| 155 | 2-[4,6-bis(trifluoromethyl)pyrimidin-2-yl]-6-chloro-1-(2,3-difluoro-4-methylphenyl)- |
| | 7-fluoro-2,3,4,9-tetrahydro-1H-pyrido[3,4-b]indole; |
| 156 | 3-(4-{6-chloro-7-fluoro-2-[4-(trifluoromethyl)pyrimidin-2-yl]-2,3,4,9-tetrahydro-1H- |
| | pyrido[3,4-b]indol-1-yl}phenyl)propan-1-ol; |
| 157 | 2-[4,6-bis(trifluoromethyl)pyrimidin-2-yl]-6-chloro-1-(2,3-difluorophenyl)-7-fluoro- |
| | 2,3,4,9-tetrahydro-1H-pyrido[3,4-b]indole; |
| 158 | 5-{6-chloro-7-fluoro-2-[4-(trifluoromethyl)pyrimidin-2-yl]-2,3,4,9-tetrahydro-1H- |
| | pyrido[3,4-b]indol-1-yl}-3-fluoro-N,N-dimethylpyridin-2-amine; |
| 159 | 3-{4-[6-chloro-7-fluoro-2-(5-fluoropyrimidin-2-yl)-2,3,4,9-tetrahydro-1H-pyrido[3,4- |
| | b]indol-1-yl]phenyl}propan-1-ol; |
| 160 | 5-{6-chloro-5-fluoro-2-[4-(trifluoromethyl)pyrimidin-2-yl]-2,3,4,9-tetrahydro-1H- |
| | pyrido[3,4-b]indol-1-yl}-3-fluoro-N,N-dimethylpyridin-2-amine; |
| 161 | (1S)-6-chloro-1-(4-methylphenyl)-2-[4-(trifluoromethyl)pyrimidin-2-yl]-2,3,4,9- |
| | tetrahydro-1H-pyrido[3,4-b]indole; |
| 162 | 2-[6-chloro-1-(2,3-difluoro-4-methylphenyl)-7-fluoro-1,3,4,9-tetrahydro-2H- |
| | pyrido[3,4-b]indol-2-yl]-5-fluoropyrimidine-4-carbonitrile; |
| 163 | 6-chloro-7-fluoro-1-(4-methylphenyl)-2-(4,5,6-trimethylpyrimidin-2-yl)-2,3,4,9- |
| | tetrahydro-1H-pyrido[3,4-b]indole; |
| 164 | methyl 3-(4-{6-chloro-7-fluoro-2-[4-(trifluoromethyl)pyrimidin-2-yl]-2,3,4,9- |
| | tetrahydro-1H-pyrido[3,4-b]indol-1-yl}phenyl)propanoate; |
| 165 | methyl 3-{4-[6-chloro-7-fluoro-2-(5-fluoropyrimidin-2-yl)-2,3,4,9-tetrahydro-1H- |
| | pyrido[3,4-b]indol-1-yl]phenyl}propanoate; |
| 166 | (4-{6-chloro-7-fluoro-2-[4-(trifluoromethyl)pyrimidin-2-yl]-2,3,4,9-tetrahydro-1H- |
| | pyrido[3,4-b]indol-1-yl}phenyl)methanol; |
| 167 | (3E)-4-(4-{6-chloro-7-fluoro-2-[4-(trifluoromethyl)pyrimidin-2-yl]-2,3,4,9- |
| | tetrahydro-1H-pyrido[3,4-b]indol-1-yl}phenyl)but-3-en-1-ol; |
| 168 | 4-(4-{6-chloro-7-fluoro-2-[4-(trifluoromethyl)pyrimidin-2-yl]-2,3,4,9-tetrahydro-1H- |
| | pyrido[3,4-b]indol-1-yl}phenyl)butan-1-ol; |
| 169 | 6-chloro-1-(2,3-difluoro-4-methylphenyl)-7-fluoro-2-(4,5,6-trimethylpyrimidin-2-yl)- |
| | 2,3,4,9-tetrahydro-1H-pyrido[3,4-b]indole; |
| 170 | 4-{6-chloro-7-fluoro-2-[4-(trifluoromethyl)pyrimidin-2-yl]-2,3,4,9-tetrahydro-1H- |
| | pyrido[3,4-b]indol-1-yl}-N,N-dimethylaniline; |
| 171 | 6-chloro-1-(2,3-difluorophenyl)-7-fluoro-2-(4,5,6-trimethylpyrimidin-2-yl)-2,3,4,9- |
| | tetrahydro-1H-pyrido[3,4-b]indole; |
| 172 | 1-(4-bromo-2,3-difluorophenyl)-6-chloro-7-fluoro-2-[4-(trifluoromethyl)pyrimidin-2- |
| | yl]-2,3,4,9-tetrahydro-1H-pyrido[3,4-b]indole; |
| 173 | 4-{6-chloro-7-fluoro-2-[4-(trifluoromethyl)pyrimidin-2-yl]-2,3,4,9-tetrahydro-1H- |
| | pyrido[3,4-b]indol-1-yl}-2,3-difluoro-N,N-dimethylaniline; |
| 174 | 5-fluoro-6-methyl-1-(4-methylphenyl)-2-[4-(trifluoromethyl)pyrimidin-2-yl]-2,3,4,9- |
| | tetrahydro-1H-pyrido[3,4-b]indole; |
| 175 | 2-[6-chloro-7-fluoro-1-(4-methylphenyl)-1,3,4,9-tetrahydro-2H-pyrido[3,4-b]indol-2- |
| | yl]-5-fluoropyrimidine-4-carbonitrile; |
| 176 | 2-[(1R)-6-chloro-1-(2,3-difluoro-4-methylphenyl)-1,3,4,9-tetrahydro-2H-pyrido[3,4- |
| | b]indol-2-yl]pyrimidine-4-carbonitrile; |
| 177 | 5-{6-chloro-5-fluoro-2-[4-(trifluoromethyl)pyrimidin-2-yl]-2,3,4,9-tetrahydro-1H- |
| | pyrido[3,4-b]indol-1-yl}-N,N-dimethylpyridin-2-amine; |
| 178 | 5-{6-chloro-5-fluoro-2-[4-(trifluoromethyl)pyrimidin-2-yl]-2,3,4,9-tetrahydro-1H- |
| | pyrido[3,4-b]indol-1-yl}-N,N-dimethylpyrimidin-2-amine; |
| 179 | (1R)-6-chloro-1-(2,3-difluoro-4-methylphenyl)-2-(5-fluoropyrimidin-2-yl)-2,3,4,9- |
| | tetrahydro-1H-pyrido[3,4-b]indole; |
| 180 | (1R)-6-chloro-1-(2,3-difluoro-4-methylphenyl)-2-[4-(trifluoromethyl)pyrimidin-2-yl]- |
| | 2,3,4,9-tetrahydro-1H-pyrido[3,4-b]indole; |
| 181 | (1R)-6-chloro-1-(4-chlorophenyl)-5-fluoro-2-[4-(trifluoromethyl)pyrimidin-2-yl]- |
| | 2,3,4,9-tetrahydro-1H-pyrido[3,4-b]indole; |
| 182 | (1S)-6-chloro-1-(4-chlorophenyl)-5-fluoro-2-[4-(trifluoromethyl)pyrimidin-2-yl]- |
| | 2,3,4,9-tetrahydro-1H-pyrido[3,4-b]indole; |
| 183 | 2-[6-chloro-1-(2,3-difluorophenyl)-7-fluoro-1,3,4,9-tetrahydro-2H-pyrido[3,4-b]indol- |
| | 2-yl]-5-fluoropyrimidine-4-carbonitrile; |
| 184 | 6-chloro-1-(2,6-difluoropyridin-3-yl)-7-fluoro-2-[4-(trifluoromethyl)pyrimidin-2-yl]- |
| | 2,3,4,9-tetrahydro-1H-pyrido[3,4-b]indole; |
| 185 | (1R)-6-chloro-1-(2,3-difluoro-4-methylphenyl)-2-(4-methylpyrimidin-2-yl)-2,3,4,9- |
| | tetrahydro-1H-pyrido[3,4-b]indole; |
| 186 | 6-chloro-1-(2,6-difluoropyridin-3-yl)-5-fluoro-2-[4-(trifluoromethyl)pyrimidin-2-yl]- |
| | 2,3,4,9-tetrahydro-1H-pyrido[3,4-b]indole; |
| 187 | (1R)-6-chloro-1-(2,3-difluoro-4-methylphenyl)-2-(5-fluoro-4-methylpyrimidin-2-yl)- |
| | 2,3,4,9-tetrahydro-1H-pyrido[3,4-b]indole; |
| 188 | 4-{2-[4,6-bis(trifluoromethyl)pyrimidin-2-yl]-6-chloro-7-fluoro-2,3,4,9-tetrahydro- |
| | 1H-pyrido[3,4-b]indol-1-yl}benzonitrile; |
| 189 | (1S)-6-chloro-1-(2,3-difluoro-4-methylphenyl)-5-fluoro-2-(5-fluoro-4,6- |
| | dimethylpyrimidin-2-yl)-2,3,4,9-tetrahydro-1H-pyrido[3,4-b]indole; |
| 190 | (1R)-6-chloro-1-(2,3-difluoro-4-methylphenyl)-5-fluoro-2-(5-fluoro-4,6- |
| | dimethylpyrimidin-2-yl)-2,3,4,9-tetrahydro-1H-pyrido[3,4-b]indole; |
| 191 | 6-chloro-5-fluoro-1-(pyrimidin-5-yl)-2-[4-(trifluoromethyl)pyrimidin-2-yl]-2,3,4,9- |
| | tetrahydro-1H-pyrido[3,4-b]indole; |
| 192 | 1-(2,3-difluoro-4-methylphenyl)-5-fluoro-6-methyl-2-[4-(trifluoromethyl)pyrimidin-2- |
| | yl]-2,3,4,9-tetrahydro-1H-pyrido[3,4-b]indole; |
| 193 | 6-chloro-5-fluoro-1-[2-(1H-imidazol-1-yl)pyrimidin-5-yl]-2-[4- |
| | (trifluoromethyl)pyrimidin-2-yl]-2,3,4,9-tetrahydro-1H-pyrido[3,4-b]indole; |
| 194 | 5-[6-chloro-5-fluoro-2-(5-fluoropyrimidin-2-yl)-2,3,4,9-tetrahydro-1H-pyrido[3,4- |
| | b]indol-1-yl]-N,N-dimethylpyridin-2-amine; |
| 195 | 6-chloro-2-(5-chloropyrimidin-2-yl)-1-(2,3-difluoro-4-methylphenyl)-5-fluoro- |
| | 2,3,4,9-tetrahydro-1H-pyrido[3,4-b]indole; |
| 196 | 6-chloro-1-(2,3-difluoro-4-methylphenyl)-5-fluoro-2-(4-methylpyrimidin-2-yl)- |
| | 2,3,4,9-tetrahydro-1H-pyrido[3,4-b]indole; |
| 197 | 6-chloro-1-(2,3-difluoro-4-methylphenyl)-5-fluoro-2-[4-(trifluoromethyl)pyrimidin-2- |
| | yl]-2,3,4,9-tetrahydro-1H-pyrido[3,4-b]indole; |
| 198 | 2-[6-chloro-1-(2,3-difluoro-4-methylphenyl)-5-fluoro-1,3,4,9-tetrahydro-2H- |
| | pyrido[3,4-b]indol-2-yl]pyrimidine-4-carbonitrile; |
| 199 | 5-{6-chloro-5-fluoro-2-[4-(trifluoromethyl)pyrimidin-2-yl]-2,3,4,9-tetrahydro-1H- |
| | pyrido[3,4-b]indol-1-yl}-N-methylpyrimidin-2-amine; |
| 200 | 6-chloro-5-fluoro-1-[2-(morpholin-4-yl)pyrimidin-5-yl]-2-[4- |
| | (trifluoromethyl)pyrimidin-2-yl]-2,3,4,9-tetrahydro-1H-pyrido[3,4-b]indole; |
| 201 | 5-[6-chloro-5-fluoro-2-(5-fluoropyrimidin-2-yl)-2,3,4,9-tetrahydro-1H-pyrido[3,4- |
| | b]indol-1-yl]-N,N-dimethylpyrimidin-2-amine; |
| 202 | 6-chloro-5-fluoro-1-(pyridin-3-yl)-2-[4-(trifluoromethyl)pyrimidin-2-yl]-2,3,4,9- |
| | tetrahydro-1H-pyrido[3,4-b]indole; |
| 203 | 6-chloro-1-(2,3-difluoro-4-methylphenyl)-2-(4-methylpyrimidin-2-yl)-2,3,4,9- |
| | tetrahydro-1H-pyrido[3,4-b]indole; |
| 204 | (1R)-6-chloro-1-(2,3-difluoro-4-methylphenyl)-2-[4-methyl-6- |
| | (trifluoromethyl)pyrimidin-2-yl]-2,3,4,9-tetrahydro-1H-pyrido[3,4-b]indole; |
| 205 | 6-chloro-1-(2,3-difluoro-4-methylphenyl)-2-(4,6-dimethylpyrimidin-2-yl)-5-fluoro- |
| | 2,3,4,9-tetrahydro-1H-pyrido[3,4-b]indole; |
| 206 | 6-chloro-1-(2,3-difluoro-4-methylphenyl)-5-fluoro-2-[4-methyl-6- |
| | (trifluoromethyl)pyrimidin-2-yl]-2,3,4,9-tetrahydro-1H-pyrido[3,4-b]indole; |
| 207 | 6-chloro-1-(4-methylphenyl)-2-[4-(trifluoromethyl)pyrimidin-2-yl]-2,3,4,9-tetrahydro- |
| | 1H-pyrido[3,4-b]indol-8-ol; |
| 208 | 5-{6-chloro-7-fluoro-2-[4-(trifluoromethyl)pyrimidin-2-yl]-2,3,4,9-tetrahydro-1H- |
| | pyrido[3,4-b]indol-1-yl}-6-fluoro-N,N-dimethylpyridin-2-amine; |
| 209 | 2-[4,6-bis(trifluoromethyl)pyrimidin-2-yl]-6-chloro-1-(2,3-difluoro-4-methylphenyl)- |
| | 5-fluoro-2,3,4,9-tetrahydro-1H-pyrido[3,4-b]indole; |
| 210 | 5-{6-chloro-5-fluoro-2-[4-(trifluoromethyl)pyrimidin-2-yl]-2,3,4,9-tetrahydro-1H- |
| | pyrido[3,4-b]indol-1-yl}-6-fluoro-N,N-dimethylpyridin-2-amine; |
| 211 | 6-chloro-5-fluoro-2-(5-fluoropyrimidin-2-yl)-1-[2-(morpholin-4-yl)pyrimidin-5-yl]- |
| | 2,3,4,9-tetrahydro-1H-pyrido[3,4-b]indole; |
| 212 | 5-[6-chloro-5-fluoro-2-(5-fluoropyrimidin-2-yl)-2,3,4,9-tetrahydro-1H-pyrido[3,4- |
| | b]indol-1-yl]pyrimidin-2-amine; |
| 213 | 6-chloro-1-(2,3-difluoro-4-methylphenyl)-2-[4-methyl-6-(trifluoromethyl)pyrimidin- |
| | 2-yl]-2,3,4,9-tetrahydro-1H-pyrido[3,4-b]indole; |
| 214 | 2-[6-chloro-1-(2,3-difluoro-4-methylphenyl)-5-fluoro-1,3,4,9-tetrahydro-2H- |
| | pyrido[3,4-b]indol-2-yl]-5-fluoropyrimidine-4-carbonitrile; |
| 215 | 2-[6-chloro-7-fluoro-1-(4-methylphenyl)-1,3,4,9-tetrahydro-2H-pyrido[3,4-b]indol-2- |
| | yl]pyrimidine-4,6-dicarbonitrile; |
| 216 | 2-[6-chloro-1-(2,3-difluoro-4-methylphenyl)-7-fluoro-1,3,4,9-tetrahydro-2H- |
| | pyrido[3,4-b]indol-2-yl]pyrimidine-4,6-dicarbonitrile; |
| 217 | 2-[6-chloro-5-fluoro-1-(4-methylphenyl)-1,3,4,9-tetrahydro-2H-pyrido[3,4-b]indol-2- |
| | yl]-5-fluoropyrimidine-4-carbonitrile; |
| 218 | 2-chloro-5-{6-chloro-2-[4-(trifluoromethyl)pyrimidin-2-yl]-2,3,4,9-tetrahydro-1H- |
| | pyrido[3,4-b]indol-1-yl}phenol; |
| 219 | 2-chloro-5-[6-chloro-2-(5-fluoropyrimidin-2-yl)-2,3,4,9-tetrahydro-1H-pyrido[3,4- |
| | b]indol-1-yl]phenol; |
| 220 | 6-chloro-1-(2,3-difluoro-4-methylphenyl)-2-[4-(trifluoromethyl)pyrimidin-2-yl]- |
| | 2,3,4,9-tetrahydro-1H-pyrido[3,4-b]indol-8-ol; |
| 221 | 5-chloro-2-[6-chloro-2-(5-fluoropyrimidin-2-yl)-2,3,4,9-tetrahydro-1H-pyrido[3,4- |
| | b]indol-1-yl]phenol; |
| 222 | 5-chloro-2-{6-chloro-2-[4-(trifluoromethyl)pyrimidin-2-yl]-2,3,4,9-tetrahydro-1H- |
| | pyrido[3,4-b]indol-1-yl}phenol; |
| 223 | 2-[(4-(6-chloro-7-fluoro-2-[4-(trifluoromethyl)pyrimidin-2-yl]-2,3,4,9-tetrahydro-1H- |
| | pyrido[3,4-b]indol-1-yl}phenyl)methoxy]ethan-1-ol; |
| 224 | 2-(6-chloro-7-fluoro-1-{4-[(1E)-4-hydroxybut-1-en-1-yl]phenyl}-1,3,4,9-tetrahydro- |
| | 2H-pyrido[3,4-b]indol-2-yl)pyrimidine-4-carbonitrile; |
| 225 | 5-{6-chloro-5-fluoro-2-[4-(trifluoromethyl)pyrimidin-2-yl]-2,3,4,9-tetrahydro-1H- |
| | pyrido[3,4-b]indol-1-yl}pyrimidin-2-amine; |
| 226 | 6-chloro-5-fluoro-1-(2-fluoropyridin-3-yl)-2-[4-(trifluoromethyl)pyrimidin-2-yl]- |
| | 2,3,4,9-tetrahydro-1H-pyrido[3,4-b]indole; |
| 227 | 2-[6-chloro-1-(2,3-difluorophenyl)-7-fluoro-1,3,4,9-tetrahydro-2H-pyrido[3,4-b]indol- |
| | 2-yl]pyrimidine-4,6-dicarbonitrile; |
| 228 | 2-[6-chloro-5-fluoro-1-(4-methylphenyl)-1,3,4,9-tetrahydro-2H-pyrido[3,4-b]indol-2- |
| | yl]pyrimidine-4,6-dicarbonitrile; |
| 229 | 2-[6-chloro-1-(2,3-difluoro-4-methylphenyl)-5-fluoro-1,3,4,9-tetrahydro-2H- |
| | pyrido[3,4-b]indol-2-yl]pyrimidine-4,6-dicarbonitrile; |
| 230 | 6-chloro-5-fluoro-1-[2-(1H-1,2,4-triazol-1-yl)pyrimidin-5-yl]-2-[4- |
| | (trifluoromethyl)pyrimidin-2-yl]-2,3,4,9-tetrahydro-1H-pyrido[3,4-b]indole; |
| 231 | 6-chloro-1-(4-methylphenyl)-2-[4-(trifluoromethyl)pyrimidin-2-yl]-2,3,4,9-tetrahydro- |
| | 1H-pyrido[3,4-b]indol-8-amine; |
| 232 | 2-[6-chloro-1-(2,3-difluoro-4-methylphenyl)-5-fluoro-1,3,4,9-tetrahydro-2H- |
| | pyrido[3,4-b]indol-2-yl]-6-methylpyrimidine-4-carbonitrile; |
| 233 | (1S)-6-chloro-1-(2,3-difluoro-4-methylphenyl)-2-[4-(trifluoromethyl)pyrimidin-2-yl]- |
| | 2,3,4,9-tetrahydro-1H-pyrido[3,4-b]indole; |
| 234 | (1S)-6-chloro-1-(2,3-difluoro-4-methylphenyl)-2-(4-methylpyrimidin-2-yl)-2,3,4,9- |
| | tetrahydro-1H-pyrido[3,4-b]indole; |
| 235 | 6-chloro-1-(2,3-difluoro-4-methylphenyl)-2-(5-fluoro-4-methylpyrimidin-2-yl)- |
| | 2,3,4,9-tetrahydro-1H-pyrido[3,4-b]indole; |
| 236 | 2-(6-chloro-5-fluoro-1-{4-[(1E)-4-hydroxybut-1-en-1-yl]phenyl}-1,3,4,9-tetrahydro- |
| | 2H-pyrido[3,4-b]indol-2-yl)pyrimidine-4-carbonitrile; |
| 237 | (3E)-4-{4-[6-chloro-5-fluoro-2-(4-methylpyrimidin-2-yl)-2,3,4,9-tetrahydro-1H- |
| | pyrido[3,4-b]indol-1-yl]phenyl}but-3-en-1-ol; |
| 238 | (1S)-6-chloro-1-(2,3-difluoro-4-methylphenyl)-2-(5-fluoropyrimidin-2-yl)-2,3,4,9- |
| | tetrahydro-1H-pyrido[3,4-b]indole; |
| 239 | (3E)-4-{4-[6-chloro-7-fluoro-2-(5-fluoro-4-methylpyrimidin-2-yl)-2,3,4,9-tetrahydro- |
| | 1H-pyrido[3,4-b]indol-1-yl]phenyl}but-3-en-1-ol; |
| 240 | 4-(4-[6-chloro-7-fluoro-2-(5-fluoro-4-methylpyrimidin-2-yl)-2,3,4,9-tetrahydro-1H- |
| | pyrido[3,4-b]indol-1-yl]phenyl}butan-1-ol; |
| 241 | (3E)-4-{4-[6-chloro-5-fluoro-2-(5-fluoro-4-methylpyrimidin-2-yl)-2,3,4,9-tetrahydro- |
| | 1H-pyrido[3,4-b]indol-1-yl]phenyl}but-3-en-1-ol; |
| 242 | (3E)-4-{4-[6-chloro-2-(4,6-dimethylpyrimidin-2-yl)-5-fluoro-2,3,4,9-tetrahydro-1H- |
| | pyrido[3,4-b]indol-1-yl]phenyl}but-3-en-1-ol; |
| 243 | (3E)-4-{4-[6-chloro-5-fluoro-2-(5-fluoro-4,6-dimethylpyrimidin-2-yl)-2,3,4,9- |
| | tetrahydro-1H-pyrido[3,4-b]indol-1-yl]phenyl}but-3-en-1-ol; |
| 244 | (3E)-4-{4-[6-chloro-2-(4,6-dimethylpyrimidin-2-yl)-7-fluoro-2,3,4,9-tetrahydro-1H- |
| | pyrido[3,4-b]indol-1-yl]phenyl}but-3-en-1-ol; |
| 245 | 4-{4-[6-chloro-2-(4,6-dimethylpyrimidin-2-yl)-7-fluoro-2,3,4,9-tetrahydro-1H- |
| | pyrido[3,4-b]indol-1-yl]phenyl}butan-1-ol; |
| 246 | 4-{4-[6-chloro-5-fluoro-2-(5-fluoro-4-methylpyrimidin-2-yl)-2,3,4,9-tetrahydro-1H- |
| | pyrido[3,4-b]indol-1-yl]phenyl}butan-1-ol; |
| 247 | 5-bromo-6-chloro-1-(4-chlorophenyl)-2-[4-(trifluoromethyl)pyrimidin-2-yl]-2,3,4,9- |
| | tetrahydro-1H-pyrido[3,4-b]indole; |
| 248 | 6-chloro-1-(2,3-difluoro-4-methylphenyl)-2-[4-(trifluoromethyl)pyrimidin-2-yl]- |
| | 2,3,4,9-tetrahydro-1H-pyrido[3,4-b]indol-8-amine; |
| 249 | 4-{4-[6-chloro-7-fluoro-2-(5-fluoro-4,6-dimethylpyrimidin-2-yl)-2,3,4,9-tetrahydro- |
| | 1H-pyrido[3,4-b]indol-1-yl]phenyl}butan-1-ol; |
| 250 | 6-chloro-1-(4-chlorophenyl)-2-[4-(trifluoromethyl)pyrimidin-2-yl]-2,3,4,9-tetrahydro- |
| | 1H-pyrido[3,4-b]indol-7-ol; |
| 251 | 6-chloro-1-[2-(morpholin-4-yl)pyrimidin-5-yl]-2-[4-(trifluoromethyl)pyrimidin-2-yl]- |
| | 2,3,4,9-tetrahydro-1H-pyrido[3,4-b]indole; |
| 252 | 2-{6-chloro-2-[4-(trifluoromethyl)pyrimidin-2-yl]-2,3,4,9-tetrahydro-1H-pyrido[3,4- |
| | b]indol-1-yl}-6-fluorophenol; |
| 253 | 6-chloro-1-(2,3-difluoro-4-methylphenyl)-2-(4,6-dimethyl-1,3,5-triazin-2-yl)-2,3,4,9- |
| | tetrahydro-1H-pyrido[3,4-b]indole; |
| 254 | 6-chloro-1-(2-fluoro-4-methylphenyl)-2-[4-(trifluoromethyl)pyrimidin-2-yl]-2,3,4,9- |
| | tetrahydro-1H-pyrido[3,4-b]indole; |
| 255 | 4-{6-chloro-2-[4-(trifluoromethyl)pyrimidin-2-yl]-2,3,4,9-tetrahydro-1H-pyrido[3,4- |
| | b]indol-1-yl}-3-fluorobenzonitrile; |
| 256 | 3-{6-chloro-2-[4-(trifluoromethyl)pyrimidin-2-yl]-2,3,4,9-tetrahydro-1H-pyrido[3,4- |
| | b]indol-1-yl}-2-fluorophenol; |
| 257 | 2-16-chloro-2-[4-(trifluoromethyl)pyrimidin-2-yl]-2,3,4,9-tetrahydro-1H-pyrido[3,4- |
| | b]indol-1-yl}-5-methylphenol; |
| 258 | 5-{6-chloro-2-[4-(trifluoromethyl)pyrimidin-2-yl]-2,3,4,9-tetrahydro-1H-pyrido[3,4- |
| | b]indol-1-yl}-2-methylphenol; |
| 259 | 6-chloro-1-(2-fluoro-3-methylphenyl)-2-[4-(trifluoromethyl)pyrimidin-2-yl]-2,3,4,9- |
| | tetrahydro-1H-pyrido[3,4-b]indole; |
| 260 | 6-chloro-1-[4-(1H-imidazol-1-yl)phenyl]-2-[4-(trifluoromethyl)pyrimidin-2-yl]- |
| | 2,3,4,9-tetrahydro-1H-pyrido[3,4-b]indole; |
| 261 | 6-chloro-1-[4-(1H-pyrazol-1-yl)phenyl]-2-[4-(trifluoromethyl)pyrimidin-2-yl]-2,3,4,9- |
| | tetrahydro-1H-pyrido[3,4-b]indole; |
| 262 | 6-chloro-1-[4-(morpholin-4-yl)phenyl]-2-[4-(trifluoromethyl)pyrimidin-2-yl]-2,3,4,9- |
| | tetrahydro-1H-pyrido[3,4-b]indole; |
| 263 | 6-chloro-2-[4-(trifluoromethyl)pyrimidin-2-yl]-1-(2,3,4-trifluorophenyl)-2,3,4,9- |
| | tetrahydro-1H-pyrido[3,4-b]indole; |
| 264 | 6-bromo-1-(4-methylphenyl)-2-[4-(trifluoromethyl)pyrimidin-2-yl]-2,3,4,9- |
| | tetrahydro-1H-pyrido[3,4-b]indole; |
| 265 | 1-(4-methylphenyl)-6-nitro-2-[4-(trifluoromethyl)pyrimidin-2-yl]-2,3,4,9-tetrahydro- |
| | 1H-pyrido[3,4-b]indole; |
| 266 | 6-chloro-1-(2,3-difluoro-4-methylphenyl)-5-nitro-2-[4-(trifluoromethyl)pyrimidin-2- |
| | yl]-2,3,4,9-tetrahydro-1H-pyrido[3,4-b]indole; |
| 267 | 6-chloro-1-(4-chloro-2-fluorophenyl)-2-[4-(trifluoromethyl)pyrimidin-2-yl]-2,3,4,9- |
| | tetrahydro-1H-pyrido[3,4-b]indole; |
| 268 | 1-(4-bromo-2-fluorophenyl)-6-chloro-2-[4-(trifluoromethyl)pyrimidin-2-yl]-2,3,4,9- |
| | tetrahydro-1H-pyrido[3,4-b]indole; |
| 269 | 6-chloro-1-[4-(4H-1,2,4-triazol-4-yl)phenyl]-2-[4-(trifluoromethyl)pyrimidin-2-yl]- |
| | 2,3,4,9-tetrahydro-1H-pyrido[3,4-b]indole; |
| 270 | 1-(4-bromo-2,3-difluorophenyl)-6-chloro-2-[4-(trifluoromethyl)pyrimidin-2-yl]- |
| | 2,3,4,9-tetrahydro-1H-pyrido[3,4-b]indole; |
| 271 | 1-(2,3-difluoro-4-methylphenyl)-6-methyl-2-[4-(trifluoromethyl)pyrimidin-2-yl]- |
| | 2,3,4,9-tetrahydro-1H-pyrido[3,4-b]indole; |
| 272 | 1-(2,3-difluoro-4-methylphenyl)-6-methyl-2-[4-(trifluoromethyl)pyrimidin-2-yl]- |
| | 2,3,4,9-tetrahydro-1H-pyrido[3,4-b]indol-8-amine; |
| 273 | 6-chloro-1-(2,3-difluoro-4-methylphenyl)-2-[4-(trifluoromethyl)pyrimidin-2-yl]- |
| | 2,3,4,9-tetrahydro-1H-pyrido[3,4-b]indol-5-amine; |
| 274 | 6-chloro-1-[2-fluoro-4-(1H-imidazol-1-yl)phenyl]-2-(pyrimidin-2-yl)-2,3,4,9- |
| | tetrahydro-1H-pyrido[3,4-b]indole; |
| 275 | 6-chloro-1-[2-fluoro-4-(1H-imidazol-1-yl)phenyl]-2-[4-(trifluoromethyl)pyrimidin-2- |
| | yl]-2,3,4,9-tetrahydro-1H-pyrido[3,4-b]indole; |
| 276 | 1-(2,3-difluoro-4-methylphenyl)-6-methyl-2-[4-(trifluoromethyl)pyrimidin-2-yl]- |
| | 2,3,4,9-tetrahydro-1H-pyrido[3,4-b]indol-8-ol; |
| 277 | 6-chloro-1-[2-fluoro-4-(morpholin-4-yl)phenyl]-2-[4-(trifluoromethyl)pyrimidin-2- |
| | yl]-2,3,4,9-tetrahydro-1H-pyrido[3,4-b]indole; |
| 278 | 4-{6-chloro-2-[4-(trifluoromethyl)pyrimidin-2-yl]-2,3,4,9-tetrahydro-1H-pyrido[3,4- |
| | b]indol-1-yl}-2,3-difluorobenzonitrile; |
| 279 | 6-chloro-1-[2,3-difluoro-4-(morpholin-4-yl)phenyl]-2-[4-(trifluoromethyl)pyrimidin- |
| | 2-yl]-2,3,4,9-tetrahydro-1H-pyrido[3,4-b]indole; |
| 280 | butyl 2-[6-chloro-1-(4-methylphenyl)-1,3,4,9-tetrahydro-2H-pyrido[3,4-b]indol-2- |
| | yl]pyrimidine-4-carboxylate; |
| 281 | butyl 2-[6-chloro-1-(2,3-difluoro-4-methylphenyl)-1,3,4,9-tetrahydro-2H-pyrido[3,4- |
| | b]indol-2-yl]pyrimidine-4-carboxylate; |
| 282 | 6-chloro-1-(2,3-difluoro-4-methylphenyl)-2-[4-(trifluoromethyl)pyrimidin-2-yl]- |
| | 2,3,4,9-tetrahydro-1H-pyrido[3,4-b]indol-7-amine; |
| 283 | 1-(4-bromo-2,3-difluorophenyl)-6-chloro-2-(4,6-dimethyl-1,3,5-triazin-2-yl)-2,3,4,9- |
| | tetrahydro-1H-pyrido[3,4-b]indole; |
| 284 | 6-chloro-1-[2,3-difluoro-4-(morpholin-4-yl)phenyl]-2-(4,6-dimethyl-1,3,5-triazin-2- |
| | yl)-2,3,4,9-tetrahydro-1H-pyrido[3,4-b]indole; |
| 285 | 6-chloro-1-[2,3-difluoro-4-(1H-imidazol-1-yl)phenyl]-2-[4- |
| | (trifluoromethyl)pyrimidin-2-yl]-2,3,4,9-tetrahydro-1H-pyrido[3,4-b]indole; |
| 286 | 6-chloro-2-(4,6-dimethylpyrimidin-2-yl)-1-(4-methylphenyl)-2,3,4,9-tetrahydro-1H- |
| | pyrido[3,4-b]indole; |
| 287 | 1-(4-methylphenyl)-6-(2H-1,2,3-triazol-2-yl)-2-[4-(trifluoromethyl)pyrimidin-2-yl]- |
| | 2,3,4,9-tetrahydro-1H-pyrido[3,4-b]indole; |
| 288 | 1-[2-(morpholin-4-yl)pyrimidin-5-yl]-6-(2H-1,2,3-triazol-2-yl)-2-[4- |
| | (trifluoromethyl)pyrimidin-2-yl]-2,3,4,9-tetrahydro-1H-pyrido[3,4-b]indole; |
| 289 | 6-chloro-2-(4-chloro-6-methyl-1,3,5-triazin-2-yl)-1-[2-(morpholin-4-yl)pyrimidin-5- |
| | yl]-2,3,4,9-tetrahydro-1H-pyrido[3,4-b]indole; |
| 290 | 4-{6-chloro-1-[2-(morpholin-4-yl)pyrimidin-5-yl]-1,3,4,9-tetrahydro-2H-pyrido[3,4- |
| | b]indol-2-yl}-N,N,6-trimethyl-1,3,5-triazin-2-amine; |
| 291 | 6-ethenyl-1-(4-methylphenyl)-2-[4-(trifluoromethyl)pyrimidin-2-yl]-2,3,4,9- |
| | tetrahydro-1H-pyrido[3,4-b]indole; |
| 292 | 6-chloro-2-(4,6-dimethyl-1,3,5-triazin-2-yl)-1-(4-methylphenyl)-2,3,4,9-tetrahydro- |
| | 1H-pyrido[3,4-b]indol-8-amine; |
| 293 | 6-chloro-2-(4,6-dimethyl-1,3,5-triazin-2-yl)-1-[2-(morpholin-4-yl)pyrimidin-5-yl]- |
| | 2,3,4,9-tetrahydro-1H-pyrido[3,4-b]indole; |
| 294 | 6-chloro-2-(4-methyl-1,3,5-triazin-2-yl)-1-[2-(morpholin-4-yl)pyrimidin-5-yl]-2,3,4,9- |
| | tetrahydro-1H-pyrido[3,4-b]indole; |
| 295 | 6-chloro-2-(4-methoxy-6-methyl-1,3,5-triazin-2-yl)-1-[2-(morpholin-4-yl)pyrimidin- |
| | 5-yl]-2,3,4,9-tetrahydro-1H-pyrido[3,4-b]indole; |
| 296 | 6-chloro-2-[4-methyl-6-(morpholin-4-yl)-1,3,5-triazin-2-yl]-1-[2-(morpholin-4- |
| | yl)pyrimidin-5-yl]-2,3,4,9-tetrahydro-1H-pyrido[3,4-b]indole; |
| 297 | 4-{6-chloro-1-[2-(morpholin-4-yl)pyrimidin-5-yl]-1,3,4,9-tetrahydro-2H-pyrido[3,4- |
| | b]indol-2-yl}-N,N-dimethyl-1,3,5-triazin-2-amine; |
| 298 | 6-bromo-1-[2-(morpholin-4-yl)pyrimidin-5-yl]-2-[4-(trifluoromethyl)pyrimidin-2-yl]- |
| | 2,3,4,9-tetrahydro-1H-pyrido[3,4-b]indole; |
| 299 | 6-bromo-2-(4,6-dimethyl-1,3,5-triazin-2-yl)-1-[2-(morpholin-4-yl)pyrimidin-5-yl]- |
| | 2,3,4,9-tetrahydro-1H-pyrido[3,4-b]indole; |
| 300 | 2-(4,6-dimethyl-1,3,5-triazin-2-yl)-1-[2-(morpholin-4-yl)pyrimidin-5-yl]-6-(2H-1,2,3- |
| | triazol-2-yl)-2,3,4,9-tetrahydro-1H-pyrido[3,4-b]indole; |
| 301 | 1-[2-(morpholin-4-yl)pyrimidin-5-yl]-6-(1H-pyrazol-1-yl)-2-[4- |
| | (trifluoromethyl)pyrimidin-2-yl]-2,3,4,9-tetrahydro-1H-pyrido[3,4-b]indole; |
| 302 | 6-methoxy-1-[2-(morpholin-4-yl)pyrimidin-5-yl]-2-[4-(trifluoromethyl)pyrimidin-2- |
| | yl]-2,3,4,9-tetrahydro-1H-pyrido[3,4-b]indole; |
| 303 | 6-chloro-1-[6-(morpholin-4-yl)pyridin-3-yl]-2-[4-(trifluoromethyl)pyrimidin-2-yl]- |
| | 2,3,4,9-tetrahydro-1H-pyrido[3,4-b]indole; |
| 304 | 6-chloro-1-(2,3-difluoro-4-methylphenyl)-2-(4,6-dimethyl-1,3,5-triazin-2-yl)-2,3,4,9- |
| | tetrahydro-1H-pyrido[3,4-b]indol-8-amine; |
| 305 | 1-(2,3-difluoro-4-methylphenyl)-6-nitro-2-[4-(trifluoromethyl)pyrimidin-2-yl]- |
| | 2,3,4,9-tetrahydro-1H-pyrido[3,4-b]indole; |
| 306 | 2-[6-chloro-1-(2,3-difluoro-4-methylphenyl)-1,3,4,9-tetrahydro-2H-pyrido[3,4- |
| | b]indol-2-yl]-N,N-dimethylpyrimidin-4-amine; |
| 307 | 2-[6-chloro-1-(4-methylphenyl)-1,3,4,9-tetrahydro-2H-pyrido[3,4-b]indol-2-yl]-N,N- |
| | dimethylpyrimidin-4-amine; |
| 308 | 6-chloro-2-(4,6-dimethylpyrimidin-2-yl)-1-[2-(morpholin-4-yl)pyrimidin-5-yl]- |
| | 2,3,4,9-tetrahydro-1H-pyrido[3,4-b]indole; |
| 309 | 6-chloro-2-(4,6-dimethyl-1,3,5-triazin-2-yl)-1-[2-(morpholin-4-yl)pyrimidin-5-yl]- |
| | 2,3,4,9-tetrahydro-1H-pyrido[3,4-b]indol-8-amine; |
| 310 | 6-chloro-1-[2-(morpholin-4-yl)pyrimidin-5-yl]-2-[4-(trifluoromethyl)pyrimidin-2-yl]- |
| | 2,3,4,9-tetrahydro-1H-pyrido[3,4-b]indol-8-amine; |
| 311 | 6-chloro-1-[2-(morpholin-4-yl)pyrimidin-5-yl]-2-[4-(trifluoromethyl)pyrimidin-2-yl]- |
| | 2,3,4,9-tetrahydro-1H-pyrido[3,4-b]indol-7-amine; |
| 312 | 6-chloro-2-(4-methylpyrimidin-2-yl)-1-[2-(morpholin-4-yl)pyrimidin-5-yl]-2,3,4,9- |
| | tetrahydro-1H-pyrido[3,4-b]indole; |
| 313 | 2-(4,6-dimethyl-1,3,5-triazin-2-yl)-1-[2-(morpholin-4-yl)pyrimidin-5-yl]-6-nitro- |
| | 2,3,4,9-tetrahydro-1H-pyrido[3,4-b]indole; |
| 314 | 1-[2-(morpholin-4-yl)pyrimidin-5-yl]-6-(1H-pyrrol-1-yl)-2-[4- |
| | (trifluoromethyl)pyrimidin-2-yl]-2,3,4,9-tetrahydro-1H-pyrido[3,4-b]indole; |
| 315 | N,N-dibutyl-4-{6-chloro-1-[2-(morpholin-4-yl)pyrimidin-5-yl]-1,3,4,9-tetrahydro-2H- |
| | pyrido[3,4-b]indol-2-yl}-6-methyl-1,3,5-triazin-2-amine; |
| 316 | 1-[6-(morpholin-4-yl)pyridin-3-yl]-6-(2H-1,2,3-triazol-2-yl)-2-[4- |
| | (trifluoromethyl)pyrimidin-2-yl]-2,3,4,9-tetrahydro-1H-pyrido[3,4-b]indole; |
| 317 | N-butyl-4-{6-chloro-1-[2-(morpholin-4-yl)pyrimidin-5-yl]-1,3,4,9-tetrahydro-2H- |
| | pyrido[3,4-b]indol-2-yl}-N,6-dimethyl-1,3,5-triazin-2-amine; |
| 318 | 2-(4-butoxy-6-methyl-1,3,5-triazin-2-yl)-6-chloro-1-[2-(morpholin-4-yl)pyrimidin-5- |
| | yl]-2,3,4,9-tetrahydro-1H-pyrido[3,4-b]indole; |
| 319 | 6-chloro-1-(2,6-dichloropyridin-3-yl)-2-[4-(trifluoromethyl)pyrimidin-2-yl]-2,3,4,9- |
| | tetrahydro-1H-pyrido[3,4-b]indole; |
| 320 | 6-chloro-2-(4,6-dimethyl-1,3,5-triazin-2-yl)-1-[2-(morpholin-4-yl)pyrimidin-5-yl]- |
| | 2,3,4,9-tetrahydro-1H-pyrido[3,4-b]indol-7-amine; |
| 321 | 6-chloro-2-(4,6-dimethyl-1,3,5-triazin-2-yl)-1-[6-(morpholin-4-yl)pyridin-3-yl]- |
| | 2,3,4,9-tetrahydro-1H-pyrido[3,4-b]indol-8-amine; |
| 322 | 6-chloro-1-(4-methylphenyl)-2-(4-methyl-1,3,5-triazin-2-yl)-2,3,4,9-tetrahydro-1H- |
| | pyrido[3,4-b]indole; |
| 323 | 1-[2-(morpholin-4-yl)pyrimidin-5-yl]-6-nitro-2-[4-(trifluoromethyl)pyrimidin-2-yl]- |
| | 2,3,4,9-tetrahydro-1H-pyrido[3,4-b]indole; |
| 324 | 6-chloro-2-(4,6-dimethyl-1,3,5-triazin-2-yl)-1-[6-(morpholin-4-yl)pyridin-3-yl]- |
| | 2,3,4,9-tetrahydro-1H-pyrido[3,4-b]indole; |
| 325 | 6-chloro-2-(4,6-dimethyl-1,3,5-triazin-2-yl)-1-[2-(morpholin-4-yl)pyrimidin-5-yl]-5- |
| | nitro-2,3,4,9-tetrahydro-1H-pyrido[3,4-b]indole; |
| 326 | 6-chloro-1-[2-(morpholin-4-yl)pyrimidin-5-yl]-5-nitro-2-[4- |
| | (trifluoromethyl)pyrimidin-2-yl]-2,3,4,9-tetrahydro-1H-pyrido[3,4-b]indole; |
| 327 | 6-chloro-2-(4,6-dimethyl-1,3,5-triazin-2-yl)-1-[2-(morpholin-4-yl)pyrimidin-5-yl]- |
| | 2,3,4,9-tetrahydro-1H-pyrido[3,4-b]indol-5-amine; |
| 328 | 6-chloro-1-[2-(morpholin-4-yl)pyrimidin-5-yl]-2-[4-(trifluoromethyl)pyrimidin-2-yl]- |
| | 2,3,4,9-tetrahydro-1H-pyrido[3,4-b]indol-5-amine; |
| 329 | N,N-dimethyl-1-[2-(morpholin-4-yl)pyrimidin-5-yl]-2-[4-(trifluoromethyl)pyrimidin- |
| | 2-yl]-2,3,4,9-tetrahydro-1H-pyrido[3,4-b]indol-6-amine; |
| 330 | 1-(2,3-difluoro-4-methylphenyl)-N,N-dimethyl-2-[4-(trifluoromethyl)pyrimidin-2-yl]- |
| | 2,3,4,9-tetrahydro-1H-pyrido[3,4-b]indol-6-amine; |
| 331 | 2-(4,6-dimethyl-1,3,5-triazin-2-yl)-N,N-dimethyl-1-[2-(morpholin-4-yl)pyrimidin-5- |
| | yl]-2,3,4,9-tetrahydro-1H-pyrido[3,4-b]indol-6-amine; |
| 332 | 6-chloro-1-(4-methylphenyl)-2-(4-methylpyrimidin-2-yl)-2,3,4,9-tetrahydro-1H- |
| | pyrido[3,4-b]indole; |
| 333 | 2-(4,6-dimethyl-1,3,5-triazin-2-yl)-1-[2-(morpholin-4-yl)pyrimidin-5-yl]-6-nitro- |
| | 2,3,4,9-tetrahydro-1H-pyrido[3,4-b]indol-8-ol; |
| 334 | 6-chloro-2-[4-(difluoromethyl)pyrimidin-2-yl]-1-[2-(morpholin-4-yl)pyrimidin-5-yl]- |
| | 2,3,4,9-tetrahydro-1H-pyrido[3,4-b]indole; |
| 335 | 6-chloro-1-[2-(2-oxa-5-azabicyclo[2.2.1]heptan-5-yl)pyrimidin-5-yl]-2-[4- |
| | (trifluoromethyl)pyrimidin-2-yl]-2,3,4,9-tetrahydro-1H-pyrido[3,4-b]indole; |
| 336 | 6-chloro-2-(4,6-dimethyl-1,3,5-triazin-2-yl)-1-[2-(2-oxa-5-azabicyclo[2.2.1]heptan-5- |
| | yl)pyrimidin-5-yl]-2,3,4,9-tetrahydro-1H-pyrido[3,4-b]indole; |
| 337 | 6-chloro-1-[2-(3-oxa-8-azabicyclo[3.2.1]octan-8-yl)pyrimidin-5-yl]-2-[4- |
| | (trifluoromethyl)pyrimidin-2-yl]-2,3,4,9-tetrahydro-1H-pyrido[3,4-b]indole; |
| 338 | 6-chloro-2-(4,6-dimethyl-1,3,5-triazin-2-yl)-1-[2-(3-oxa-8-azabicyclo[3.2.1]octan-8- |
| | yl)pyrimidin-5-yl]-2,3,4,9-tetrahydro-1H-pyrido[3,4-b]indole; |
| 339 | 6-chloro-2-(4,6-dimethyl-1,3,5-triazin-2-yl)-1-[6-(morpholin-4-yl)pyridin-3-yl]- |
| | 2,3,4,9-tetrahydro-1H-pyrido[3,4-b]indol-8-ol; |
| 340 | 2-[4,6-bis(trifluoromethyl)pyrimidin-2-yl]-6-chloro-1-(2,3-difluoro-4-methylphenyl)- |
| | 2,3,4,9-tetrahydro-1H-pyrido[3,4-b]indole; |
| 341 | 6-chloro-1-(2-fluorophenyl)-2-[4-(trifluoromethyl)pyrimidin-2-yl]-2,3,4,9-tetrahydro- |
| | 1H-pyrido[3,4-b]indole; |
| 342 | 6-chloro-1-(2,3-difluoro-4-methylphenyl)-2-(4,6-dimethylpyrimidin-2-yl)-2,3,4,9- |
| | tetrahydro-1H-pyrido[3,4-b]indole; |
| 343 | 6-chloro-1-(4-methylphenyl)-2-[4-(trifluoromethyl)pyrimidin-2-yl]-2,3,4,9-tetrahydro- |
| | 1H-pyrido[3,4-b]indole; |
| 344 | 1-[2-(azetidin-1-yl)pyrimidin-5-yl]-6-chloro-2-[4-(trifluoromethyl)pyrimidin-2-yl]- |
| | 2,3,4,9-tetrahydro-1H-pyrido[3,4-b]indole; |
| 345 | 1-[2-(azetidin-1-yl)pyrimidin-5-yl]-6-chloro-2-(4,6-dimethyl-1,3,5-triazin-2-yl)- |
| | 2,3,4,9-tetrahydro-1H-pyrido[3,4-b]indole; |
| 346 | 6-chloro-2-(4,6-dimethyl-1,3,5-triazin-2-yl)-1-[2-(methylsulfanyl)pyrimidin-5-yl]- |
| | 2,3,4,9-tetrahydro-1H-pyrido[3,4-b]indole; |
| 347 | (1S)-6-chloro-1-(2,3-difluoro-4-methylphenyl)-2-(4,6-dimethyl-1,3,5-triazin-2-yl)- |
| | 2,3,4,9-tetrahydro-1H-pyrido[3,4-b]indole; |
| 348 | 6-chloro-1-(2,3-difluoro-4-methylphenyl)-2-(4,6-dimethyl-1,3,5-triazin-2-yl)-2,3,4,9- |
| | tetrahydro-1H-pyrido[3,4-b]indol-8-ol; |
| 349 | 6-chloro-1-(4-chlorophenyl)-2-[4-(trifluoromethyl)pyrimidin-2-yl]-2,3,4,9-tetrahydro- |
| | 1H-pyrido[3,4-b]indole; |
| 350 | 6-chloro-2-(4,6-dimethylpyrimidin-2-yl)-1-[6-(morpholin-4-yl)pyridin-3-yl]-2,3,4,9- |
| | tetrahydro-1H-pyrido[3,4-b]indole; |
| 351 | 6-chloro-2-(4-methylpyrimidin-2-yl)-1-[6-(morpholin-4-yl)pyridin-3-yl]-2,3,4,9- |
| | tetrahydro-1H-pyrido[3,4-b]indol-8-amine; |
| 352 | 6-chloro-2-(4-methylpyrimidin-2-yl)-1-[6-(morpholin-4-yl)pyridin-3-yl]-2,3,4,9- |
| | tetrahydro-1H-pyrido[3,4-b]indole; |
| 353 | 2-{6-chloro-2-[4-(trifluoromethyl)pyrimidin-2-yl]-2,3,4,9-tetrahydro-1H-pyrido[3,4- |
| | b]indol-1-yl}phenol; |
| 354 | 6-chloro-1-(3-fluoro-4-methylphenyl)-2-[4-(trifluoromethyl)pyrimidin-2-yl]-2,3,4,9- |
| | tetrahydro-1H-pyrido[3,4-b]indole; |
| 355 | 6-chloro-2-(4,6-dimethyl-1,3,5-triazin-2-yl)-1-(4-methylphenyl)-2,3,4,9-tetrahydro- |
| | 1H-pyrido[3,4-b]indole; |
| 356 | 2-{6-chloro-1-[6-(morpholin-4-yl)pyridin-3-yl]-1,3,4,9-tetrahydro-2H-pyrido[3,4- |
| | b]indol-2-yl}pyrimidine-4-carbonitrile; |
| ¹357 | 2-[4,6-bis(trifluoromethyl)pyrimidin-2-yl]-6-chloro-1-[6-(morpholin-4-yl)pyridin-3- |
| | yl]-2,3,4,9-tetrahydro-1H-pyrido[3,4-b]indole; |
| ¹358 | 2-[4,6-bis(trifluoromethyl)pyrimidin-2-yl]-6-chloro-1-[2-(morpholin-4-yl)pyrimidin- |
| | 5-yl]-2,3,4,9-tetrahydro-1H-pyrido[3,4-b]indole; |
| 359 | 2-{6-chloro-1-[2-(morpholin-4-yl)pyrimidin-5-yl]-1,3,4,9-tetrahydro-2H-pyrido[3,4- |
| | b]indol-2-yl}pyrimidine-4-carbonitrile; |
| 360 | 2-[4,6-bis(trifluoromethyl)pyrimidin-2-yl]-6-chloro-1-(4-methylphenyl)-2,3,4,9- |
| | tetrahydro-1H-pyrido[3,4-b]indole; |
| 361 | 6-chloro-1-(4-methylphenyl)-2-[4-methyl-6-(trifluoromethyl)pyrimidin-2-yl]-2,3,4,9- |
| | tetrahydro-1H-pyrido[3,4-b]indole; |
| 362 | 2-{6-chloro-1-[2-(morpholin-4-yl)pyrimidin-5-yl]-1,3,4,9-tetrahydro-2H-pyrido[3,4- |
| | b]indol-2-yl}-6-methylpyrimidine-4-carbonitrile; |
| 363 | 2-[6-chloro-1-(2,3-difluoro-4-methylphenyl)-1,3,4,9-tetrahydro-2H-pyrido[3,4- |
| | b]indol-2-yl]pyrimidine-4-carbonitrile; |
| 364 | 6-chloro-2-(5-fluoropyrimidin-2-yl)-1-[2-(morpholin-4-yl)pyrimidin-5-yl]-2,3,4,9- |
| | tetrahydro-1H-pyrido[3,4-b]indole; |
| 365 | 6-chloro-2-(5-fluoro-4-methylpyrimidin-2-yl)-1-[6-(morpholin-4-yl)pyridin-3-yl]- |
| | 2,3,4,9-tetrahydro-1H-pyrido[3,4-b]indole; |
| 366 | 6-chloro-2-(5-fluoro-4-methylpyrimidin-2-yl)-1-[2-(morpholin-4-yl)pyrimidin-5-yl]- |
| | 2,3,4,9-tetrahydro-1H-pyrido[3,4-b]indole; |
| 367 | 6-chloro-2-(5-fluoro-4,6-dimethylpyrimidin-2-yl)-1-[6-(morpholin-4-yl)pyridin-3-yl]- |
| | 2,3,4,9-tetrahydro-1H-pyrido[3,4-b]indole; |
| 368 | 6-chloro-2-(5-fluoro-4,6-dimethylpyrimidin-2-yl)-1-[2-(morpholin-4-yl)pyrimidin-5- |
| | yl]-2,3,4,9-tetrahydro-1H-pyrido[3,4-b]indole; |
| 369 | 6-chloro-1-[2-(4-methylpiperazin-1-yl)pyrimidin-5-yl]-2-[4- |
| | (trifluoromethyl)pyrimidin-2-yl]-2,3,4,9-tetrahydro-1H-pyrido[3,4-b]indole; |
| 370 | 6-chloro-2-(4,6-dimethyl-1,3,5-triazin-2-yl)-1-[2-(morpholin-4-yl)pyrimidin-5-yl]- |
| | 2,3,4,9-tetrahydro-1H-pyrido[3,4-b]indol-8-ol; |
| 371 | 6-chloro-1-(3-fluorophenyl)-2-[4-(trifluoromethyl)pyrimidin-2-yl]-2,3,4,9-tetrahydro- |
| | 1H-pyrido[3,4-b]indole; |
| 372 | 6-chloro-2-[4-methyl-6-(trifluoromethyl)pyrimidin-2-yl]-1-[6-(morpholin-4- |
| | yl)pyridin-3-yl]-2,3,4,9-tetrahydro-1H-pyrido[3,4-b]indole; |
| 373 | 6-chloro-1-{6-[(1R,4R)-2-oxa-5-azabicyclo[2.2.1]heptan-5-yl]pyridin-3-yl}-2-[4- |
| | (trifluoromethyl)pyrimidin-2-yl]-2,3,4,9-tetrahydro-1H-pyrido[3,4-b]indole; |
| 374 | 6-chloro-2-[4-methyl-6-(trifluoromethyl)pyrimidin-2-yl]-1-[2-(morpholin-4- |
| | yl)pyrimidin-5-yl]-2,3,4,9-tetrahydro-1H-pyrido[3,4-b]indole; |
| 375 | 6-chloro-1-{6-[(1S,4S)-2-oxa-5-azabicyclo[2.2.1]heptan-5-yl]pyridin-3-yl}-2-[4- |
| | (trifluoromethyl)pyrimidin-2-yl]-2,3,4,9-tetrahydro-1H-pyrido[3,4-b]indole; |
| 376 | 6-chloro-2-(4,6-dimethyl-1,3,5-triazin-2-yl)-1-{6-[(1R,4R)-2-oxa-5- |
| | azabicyclo[2.2.1]heptan-5-yl]pyridin-3-yl}-2,3,4,9-tetrahydro-1H-pyrido[3,4-b]indole; |
| 377 | 6-chloro-2-(4,6-dimethyl-1,3,5-triazin-2-yl)-1-{6-[(1S,4S)-2-oxa-5- |
| | azabicyclo[2.2.1]heptan-5-yl]pyridin-3-yl}-2,3,4,9-tetrahydro-1H-pyrido[3,4-b]indole; |
| 378 | 2-[6-chloro-1-(4-methylphenyl)-1,3,4,9-tetrahydro-2H-pyrido[3,4-b]indol-2- |
| | yl]pyrimidine-4-carbonitrile; |
| 379 | 2-[6-chloro-1-(4-methylphenyl)-1,3,4,9-tetrahydro-2H-pyrido[3,4-b]indol-2-yl]-6- |
| | methylpyrimidine-4-carbonitrile; |
| 380 | 6-chloro-2-(4,6-dimethyl-1,3,5-triazin-2-yl)-1-[2-(4-methylpiperazin-1-yl)pyrimidin- |
| | 5-yl]-2,3,4,9-tetrahydro-1H-pyrido[3,4-b]indole; |
| 381 | 2-[4,6-bis(trifluoromethyl)pyrimidin-2-yl]-6-chloro-1-[2-(4-methylpiperazin-1- |
| | yl)pyrimidin-5-yl]-2,3,4,9-tetrahydro-1H-pyrido[3,4-b]indole; |
| 382 | 6-chloro-2-(5-fluoropyrimidin-2-yl)-1-[6-(morpholin-4-yl)pyridin-3-yl]-2,3,4,9- |
| | tetrahydro-1H-pyrido[3,4-b]indole; |
| 383 | 6-chloro-1-[2-(morpholin-4-yl)pyrimidin-5-yl]-2-[5-(trifluoromethyl)-1,2,4-triazin-3- |
| | yl]-2,3,4,9-tetrahydro-1H-pyrido[3,4-b]indole; |
| 384 | 6-chloro-2-(5-methyl-1,2,4-triazin-3-yl)-1-[2-(morpholin-4-yl)pyrimidin-5-yl]-2,3,4,9- |
| | tetrahydro-1H-pyrido[3,4-b]indole; |
| 385 | 2-[(1S)-6-chloro-1-(2,3-difluoro-4-methylphenyl)-1,3,4,9-tetrahydro-2H-pyrido[3,4- |
| | b]indol-2-yl]-5-fluoropyrimidine-4-carbonitrile; |
| 386 | (1S)-2-[4,6-bis(trifluoromethyl)-1,3,5-triazin-2-yl]-6-chloro-1-(4-methylphenyl)- |
| | 2,3,4,9-tetrahydro-1H-pyrido[3,4-b]indole; |
| 387 | (1S)-2-[4,6-bis(trifluoromethyl)-1,3,5-triazin-2-yl]-6-chloro-1-(2,3-difluoro-4- |
| | methylphenyl)-2,3,4,9-tetrahydro-1H-pyrido[3,4-b]indole; |
| 388 | 2-[4,6-bis(trifluoromethyl)-1,3,5-triazin-2-yl]-6-chloro-1-[2-(morpholin-4- |
| | yl)pyrimidin-5-yl]-2,3,4,9-tetrahydro-1H-pyrido[3,4-b]indole; |
| 389 | (1S)-6-chloro-1-(2,3-difluoro-4-methylphenyl)-2-[4-(methylsulfanyl)-6- |
| | (trifluoromethyl)-1,3,5-triazin-2-yl]-2,3,4,9-tetrahydro-1H-pyrido[3,4-b]indole; |
| 390 | 4-[(1S)-6-chloro-1-(2,3-difluoro-4-methylphenyl)-1,3,4,9-tetrahydro-2H-pyrido[3,4- |
| | b]indol-2-yl]-6-(trifluoromethyl)-1,3,5-triazine-2-carbonitrile; |
| 391 | 6-chloro-1-[2-(2-oxa-6-azaspiro[3.3]heptan-6-yl)pyrimidin-5-yl]-2-[4- |
| | (trifluoromethyl)pyrimidin-2-yl]-2,3,4,9-tetrahydro-1H-pyrido[3,4-b]indole; |
| 392 | 2-[(1S)-6-chloro-1-(4-methylphenyl)-1,3,4,9-tetrahydro-2H-pyrido[3,4-b]indol-2-yl]- |
| | 5-fluoropyrimidine-4-carbonitrile; |
| 393 | (1S)-6-chloro-2-(5-fluoropyrimidin-2-yl)-1-(4-methylphenyl)-2,3,4,9-tetrahydro-1H- |
| | pyrido[3,4-b]indole; |
| 394 | (1S)-6-chloro-2-(5-fluoro-4-methylpyrimidin-2-yl)-1-(4-methylphenyl)-2,3,4,9- |
| | tetrahydro-1H-pyrido[3,4-b]indole; |
| 395 | (1S)-6-chloro-2-(5-fluoro-4,6-dimethylpyrimidin-2-yl)-1-(4-methylphenyl)-2,3,4,9- |
| | tetrahydro-1H-pyrido[3,4-b]indole; |
| 396 | 2-{(1S)-6-chloro-1-[2-(morpholin-4-yl)pyrimidin-5-yl]-1,3,4,9-tetrahydro-2H- |
| | pyrido[3,4-b]indol-2-yl}-5-fluoropyrimidine-4-carbonitrile; |
| 397 | 2-[(1S)-6-chloro-1-(4-methylphenyl)-1,3,4,9-tetrahydro-2H-pyrido[3,4-b]indol-2- |
| | yl]pyrimidine-4,6-dicarbonitrile; |
| 398 | 2-{(1S)-6-chloro-1-[2-(morpholin-4-yl)pyrimidin-5-yl]-1,3,4,9-tetrahydro-2H- |
| | pyrido[3,4-b]indol-2-yl}pyrimidine-4,6-dicarbonitrile; |
| 399 | 4-[(1S)-6-chloro-1-(4-methylphenyl)-1,3,4,9-tetrahydro-2H-pyrido[3,4-b]indol-2-yl]- |
| | 1,3,5-triazine-2-carbonitrile; |
| 400 | 2-{(1S)-6-chloro-1-[6-(morpholin-4-yl)pyridin-3-yl]-1,3,4,9-tetrahydro-2H- |
| | pyrido[3,4-b]indol-2-yl}-5-fluoropyrimidine-4-carbonitrile; |
| 401 | 6-chloro-1-(2,3-difluoro-4-methylphenyl)-2-(5-fluoropyrimidin-2-yl)-2,3,4,9- |
| | tetrahydro-1H-pyrido[3,4-b]indole; |
| 402 | (1S)-6-chloro-2-(4,6-dimethyl-1,3,5-triazin-2-yl)-1-(4-methylphenyl)-2,3,4,9- |
| | tetrahydro-1H-pyrido[3,4-b]indole; |
| 403 | 6-chloro-1-(2,3-difluoro-4-methylphenyl)-2-[5-(trifluoromethyl)-1,2,4-triazin-3-yl]- |
| | 2,3,4,9-tetrahydro-1H-pyrido[3,4-b]indole; |
| 404 | 2-[4,6-bis(trifluoromethyl)-1,3,5-triazin-2-yl]-6-chloro-1-[6-(morpholin-4-yl)pyridin- |
| | 3-yl]-2,3,4,9-tetrahydro-1H-pyrido[3,4-b]indole; |
| 405 | (1S)-2-[4,6-bis(trifluoromethyl)-1,3,5-triazin-2-yl]-6-chloro-1-[6-(morpholin-4- |
| | yl)pyridin-3-yl]-2,3,4,9-tetrahydro-1H-pyrido[3,4-b]indole; |
| 406 | (1S)-2-[4,6-bis(trifluoromethyl)-1,3,5-triazin-2-yl]-6-chloro-1-[2-(morpholin-4- |
| | yl)pyrimidin-5-yl]-2,3,4,9-tetrahydro-1H-pyrido[3,4-b]indole; |
| 407 | (1S)-2-[4,6-bis(trifluoromethyl)pyrimidin-2-yl]-6-chloro-1-(2,3-difluoro-4- |
| | methylphenyl)-2,3,4,9-tetrahydro-1H-pyrido[3,4-b]indole; |
| 408 | (1S)-6-chloro-1-[2-(morpholin-4-yl)pyrimidin-5-yl]-2-[4-(trifluoromethyl)pyrimidin- |
| | 2-yl]-2,3,4,9-tetrahydro-1H-pyrido[3,4-b]indole; |
| 409 | (1S)-2-[4,6-bis(trifluoromethyl)pyrimidin-2-yl]-6-chloro-1-[6-(morpholin-4- |
| | yl)pyridin-3-yl]-2,3,4,9-tetrahydro-1H-pyrido[3,4-b]indole; |
| 410 | (1S)-2-[4,6-bis(trifluoromethyl)pyrimidin-2-yl]-6-chloro-1-[2-(morpholin-4- |
| | yl)pyrimidin-5-yl]-2,3,4,9-tetrahydro-1H-pyrido[3,4-b]indole; |
| 411 | 4-[(1S)-6-chloro-1-(4-methylphenyl)-1,3,4,9-tetrahydro-2H-pyrido[3,4-b]indol-2-yl]- |
| | 6-methyl-1,3,5-triazine-2-carbonitrile; |
| 412 | (1S)-6-chloro-1-(2,3-difluoro-4-methylphenyl)-2-(5-fluoro-4,6-dimethylpyrimidin-2- |
| | yl)-2,3,4,9-tetrahydro-1H-pyrido[3,4-b]indole; |
| 413 | 2-[(1S)-6-chloro-1-(2,3-difluoro-4-methylphenyl)-1,3,4,9-tetrahydro-2H-pyrido[3,4- |
| | b]indol-2-yl]-5-fluoro-6-methylpyrimidine-4-carbonitrile; |
| 414 | 4-{(1S)-6-chloro-1-[2-(morpholin-4-yl)pyrimidin-5-yl]-1,3,4,9-tetrahydro-2H- |
| | pyrido[3,4-b]indol-2-yl}-1,3,5-triazine-2-carbonitrile; |
| 415 | 4-{(1S)-6-chloro-1-[2-(morpholin-4-yl)pyrimidin-5-yl]-1,3,4,9-tetrahydro-2H- |
| | pyrido[3,4-b]indol-2-yl}-6-methyl-1,3,5-triazine-2-carbonitrile; |
| 416 | 2-[(1S)-6-chloro-1-(2,3-difluoro-4-methylphenyl)-1,3,4,9-tetrahydro-2H-pyrido[3,4- |
| | b]indol-2-yl]-6-(trifluoromethyl)pyrimidine-4-carbonitrile; |
| 417 | 2-[(1S)-6-chloro-1-(4-methylphenyl)-1,3,4,9-tetrahydro-2H-pyrido[3,4-b]indol-2-yl]- |
| | 5-fluoro-6-methylpyrimidine-4-carbonitrile; |
| 418 | (1S)-6-chloro-1-(4-methylphenyl)-2-[4-(trifluoromethyl)-1,3,5-triazin-2-yl]-2,3,4,9- |
| | tetrahydro-1H-pyrido[3,4-b]indole; |
| 419 | (1S)-2-[4,6-bis(trifluoromethyl)pyrimidin-2-yl]-6-chloro-1-[2-(4-methylpiperazin-1- |
| | yl)pyrimidin-5-yl]-2,3,4,9-tetrahydro-1H-pyrido[3,4-b]indole; |
| 420 | (1S)-2-[4,6-bis(trifluoromethyl)-1,3,5-triazin-2-yl]-6-chloro-1-[2-(4-methylpiperazin- |
| | 1-yl)pyrimidin-5-yl]-2,3,4,9-tetrahydro-1H-pyrido[3,4-b]indole; |
| 421 | 2-{(1S)-6-chloro-1-[2-(morpholin-4-yl)pyrimidin-5-yl]-1,3,4,9-tetrahydro-2H- |
| | pyrido[3,4-b]indol-2-yl}-S-fluoro-6-methylpyrimidine-4-carbonitrile; |
| 422 | 6-chloro-1-(4-fluorophenyl)-2-[4-(trifluoromethyl)pyrimidin-2-yl]-2,3,4,9-tetrahydro- |
| | 1H-pyrido[3,4-b]indole; |
| 423 | 6-chloro-1-(2,4-difluorophenyl)-2-[4-(trifluoromethyl)pyrimidin-2-yl]-2,3,4,9- |
| | tetrahydro-1H-pyrido[3,4-b]indole; |
| 424 | 2-{(1S)-6-chloro-1-[6-(morpholin-4-yl)pyridin-3-yl]-1,3,4,9-tetrahydro-2H- |
| | pyrido[3,4-b]indol-2-yl}-5-fluoro-6-methylpyrimidine-4-carbonitrile; |
| 425 | 2-[(1S)-6-chloro-1-(4-methylphenyl)-1,3,4,9-tetrahydro-2H-pyrido[3,4-b]indol-2-yl]- |
| | 6-(trifluoromethyl)pyrimidine-4-carbonitrile; |
| 426 | 4-[(1S)-6-chloro-1-(4-methylphenyl)-1,3,4,9-tetrahydro-2H-pyrido[3,4-b]indol-2-yl]- |
| | 6-(trifluoromethyl)-1,3,5-triazin-2-amine; |
| 427 | 4-[(1S)-6-chloro-1-(2,3-difluoro-4-methylphenyl)-1,3,4,9-tetrahydro-2H-pyrido[3,4- |
| | b]indol-2-yl]-6-(trifluoromethyl)-1,3,5-triazin-2-amine; |
| 428 | 6-chloro-1-[6-(morpholin-4-yl)pyridin-3-yl]-2-[4-(morpholin-4-yl)-6- |
| | (trifluoromethyl)-1,3,5-triazin-2-yl]-2,3,4,9-tetrahydro-1H-pyrido[3,4-b]indole; |
| 429 | 6-chloro-1-[6-(morpholin-4-yl)pyridin-3-yl]-2-[5-(trifluoromethyl)-1,2,4-triazin-3-yl]- |
| | 2,3,4,9-tetrahydro-1H-pyrido[3,4-b]indole; |
| 430 | 6-chloro-1-(4-methylphenyl)-2-[5-(trifluoromethyl)-1,2,4-triazin-3-yl]-2,3,4,9- |
| | tetrahydro-1H-pyrido[3,4-b]indole; |
| 431 | 6-chloro-1-(2,3-difluoro-4-methylphenyl)-2-[6-(trifluoromethyl)-1,2,4-triazin-3-yl]- |
| | 2,3,4,9-tetrahydro-1H-pyrido[3,4-b]indole; |
| 432 | 6-chloro-1-[2-fluoro-6-(morpholin-4-yl)pyridin-3-yl]-2-[4-(trifluoromethyl)pyrimidin- |
| | 2-yl]-2,3,4,9-tetrahydro-1H-pyrido[3,4-b]indole; |
| 433 | 2-{(1S)-6-chloro-1-[6-(morpholin-4-yl)pyridin-3-yl]-1,3,4,9-tetrahydro-2H- |
| | pyrido[3,4-b]indol-2-yl}-6-(trifluoromethyl)pyrimidine-4-carbonitrile; |
| 434 | 2-{(1S)-6-chloro-1-[2-(morpholin-4-yl)pyrimidin-5-yl]-1,3,4,9-tetrahydro-2H- |
| | pyrido[3,4-b]indol-2-yl}-6-(trifluoromethyl)pyrimidine-4-carbonitrile; |
| 435 | 4-[(1S)-6-chloro-1-(2,3-difluoro-4-methylphenyl)-1,3,4,9-tetrahydro-2H-pyrido[3,4- |
| | b]indol-2-yl]-N,N-dimethyl-6-(trifluoromethyl)-1,3,5-triazin-2-amine; |
| 436 | (1S)-6-chloro-1-(2,3-difluoro-4-methylphenyl)-2-[4-(morpholin-4-yl)-6- |
| | (trifluoromethyl)-1,3,5-triazin-2-yl]-2,3,4,9-tetrahydro-1H-pyrido[3,4-b]indole; |
| 437 | 6-chloro-1-(4-methylphenyl)-2-[4-methyl-6-(trifluoromethyl)-1,3,5-triazin-2-yl]- |
| | 2,3,4,9-tetrahydro-1H-pyrido[3,4-b]indole; |
| 438 | 6-chloro-1-(2,3-difluoro-4-methylphenyl)-2-[4-methyl-6-(trifluoromethyl)-1,3,5- |
| | triazin-2-yl]-2,3,4,9-tetrahydro-1H-pyrido[3,4-b]indole; |
| 439 | 6-chloro-2-[4-methyl-6-(trifluoromethyl)-1,3,5-triazin-2-yl]-1-[6-(morpholin-4- |
| | yl)pyridin-3-yl]-2,3,4,9-tetrahydro-1H-pyrido[3,4-b]indole; |
| 440 | 6-chloro-2-[4-methyl-6-(trifluoromethyl)-1,3,5-triazin-2-yl]-1-[2-(morpholin-4- |
| | yl)pyrimidin-5-yl]-2,3,4,9-tetrahydro-1H-pyrido[3,4-b]indole; |
| 441 | 4-[(1S)-6-chloro-1-(4-methylphenyl)-1,3,4,9-tetrahydro-2H-pyrido[3,4-b]indol-2-yl]- |
| | N,N-dimethyl-6-(trifluoromethyl)-1,3,5-triazin-2-amine; |
| 442 | (1S)-6-chloro-1-(4-methylphenyl)-2-[4-(morpholin-4-yl)-6-(trifluoromethyl)-1,3,5- |
| | triazin-2-yl]-2,3,4,9-tetrahydro-1H-pyrido[3,4-b]indole; |
| 443 | 4-{(1S)-6-chloro-1-[6-(morpholin-4-yl)pyridin-3-yl]-1,3,4,9-tetrahydro-2H- |
| | pyrido[3,4-b]indol-2-yl}-N,N-dimethyl-6-(trifluoromethyl)-1,3,5-triazin-2-amine; |
| 444 | 4-{(1S)-6-chloro-1-[2-(morpholin-4-yl)pyrimidin-5-yl]-1,3,4,9-tetrahydro-2H- |
| | pyrido[3,4-b]indol-2-yl}-N,N-dimethyl-6-(trifluoromethyl)-1,3,5-triazin-2-amine; |
| 445 | (1S)-6-chloro-1-[2-(morpholin-4-yl)pyrimidin-5-yl]-2-[4-(morpholin-4-yl)-6- |
| | (trifluoromethyl)-1,3,5-triazin-2-yl]-2,3,4,9-tetrahydro-1H-pyrido[3,4-b]indole; |
| 446 | (1S)-6-chloro-1-[6-(morpholin-4-yl)pyridin-3-yl]-2-[4-(morpholin-4-yl)-6- |
| | (trifluoromethyl)-1,3,5-triazin-2-yl]-2,3,4,9-tetrahydro-1H-pyrido[3,4-b]indole; |
| 447 | 4-{6-chloro-2-[4-(trifluoromethyl)pyrimidin-2-yl]-2,3,4,9-tetrahydro-1H-pyrido[3,4- |
| | b]indol-1-yl}benzonitrile; |
| 448 | 4-[6-chloro-2-(4,6-dimethyl-1,3,5-triazin-2-yl)-2,3,4,9-tetrahydro-1H-pyrido[3,4- |
| | b]indol-1-yl]benzonitrile; |
| 449 | 6-chloro-1-(4-chlorophenyl)-2-(4,6-dimethyl-1,3,5-triazin-2-yl)-2,3,4,9-tetrahydro- |
| | 1H-pyrido[3,4-b]indole; |
| 450 | 6-chloro-2-(4,6-dimethyl-1,3,5-triazin-2-yl)-1-(4-fluorophenyl)-2,3,4,9-tetrahydro- |
| | 1H-pyrido[3,4-b]indole; |
| 451 | 6-chloro-1-(2,4-difluorophenyl)-2-(4,6-dimethyl-1,3,5-triazin-2-yl)-2,3,4,9-tetrahydro- |
| | 1H-pyrido[3,4-b]indole; |
| 452 | 6-chloro-2-(4,6-dimethyl-1,3,5-triazin-2-yl)-1-(2-fluoro-4-methylphenyl)-2,3,4,9- |
| | tetrahydro-1H-pyrido[3,4-b]indole; |
| 453 | 4-[6-chloro-2-(4,6-dimethyl-1,3,5-triazin-2-yl)-2,3,4,9-tetrahydro-1H-pyrido[3,4- |
| | b]indol-1-yl]-3-fluorobenzonitrile; |
| 454 | 2-[4,6-bis(trifluoromethyl)pyrimidin-2-yl]-1-[6-(morpholin-4-yl)pyridin-3-yl]-6-(2H- |
| | 1,2,3-triazol-2-yl)-2,3,4,9-tetrahydro-1H-pyrido[3,4-b]indole; |
| 455 | 2-[4,6-bis(trifluoromethyl)pyrimidin-2-yl]-6-(4-methyl-2H-1,2,3-triazol-2-yl)-1-[6- |
| | (morpholin-4-yl)pyridin-3-yl]-2,3,4,9-tetrahydro-1H-pyrido[3,4-b]indole; |
| 456 | (1S)-6-chloro-2-[4-(3,3-difluoroazetidin-1-yl)pyrimidin-2-yl]-1-[2-(morpholin-4- |
| | yl)pyrimidin-5-yl]-2,3,4,9-tetrahydro-1H-pyrido[3,4-b]indole; |
| 457 | (1S)-6-chloro-2-[4-(3,3-difluoroazetidin-1-yl)-6-methyl-1,3,5-triazin-2-yl]-1-[2- |
| | (morpholin-4-yl)pyrimidin-5-yl]-2,3,4,9-tetrahydro-1H-pyrido[3,4-b]indole; |
| 458 | 6-chloro-1-[2-(morpholin-4-yl)pyrimidin-5-yl]-2-[6-(trifluoromethyl)-1,2,4-triazin-3- |
| | yl]-2,3,4,9-tetrahydro-1H-pyrido[3,4-b]indole; |
| 459 | 6-chloro-2-(4,6-dimethyl-1,3,5-triazin-2-yl)-1-[2-fluoro-6-(morpholin-4-yl)pyridin-3- |
| | yl]-2,3,4,9-tetrahydro-1H-pyrido[3,4-b]indole; |
| 460 | (1S)-6-chloro-1-[2-(morpholin-4-yl)pyrimidin-5-yl]-2-[4-(trifluoromethyl)-1,3,5- |
| | triazin-2-yl]-2,3,4,9-tetrahydro-1H-pyrido[3,4-b]indole; |
| 461 | (1S)-6-chloro-1-(2,3-difluoro-4-methylphenyl)-2-[4-(trifluoromethyl)-1,3,5-triazin-2- |
| | yl]-2,3,4,9-tetrahydro-1H-pyrido[3,4-b]indole; |
| 462 | (1S)-6-chloro-1-[6-(morpholin-4-yl)pyridin-3-yl]-2-[4-(trifluoromethyl)-1,3,5-triazin- |
| | 2-yl]-2,3,4,9-tetrahydro-1H-pyrido[3,4-b]indole; |
| 463 | (1S)-2-(5-bromopyrimidin-2-yl)-6-chloro-1-(2,3-difluoro-4-methylphenyl)-2,3,4,9- |
| | tetrahydro-1H-pyrido[3,4-b]indole; |
| 464 | (1S)-6-chloro-1-(2,3-difluoro-4-methylphenyl)-2-[4-methyl-6-(trifluoromethyl)-1,3,5- |
| | triazin-2-yl]-2,3,4,9-tetrahydro-1H-pyrido[3,4-b]indole; |
| 465 | (1S)-6-chloro-2-[4-methyl-6-(trifluoromethyl)-1,3,5-triazin-2-yl]-1-[6-(morpholin-4- |
| | yl)pyridin-3-yl]-2,3,4,9-tetrahydro-1H-pyrido[3,4-b]indole; |
| 466 | (1S)-6-chloro-2-[4-methyl-6-(trifluoromethyl)-1,3,5-triazin-2-yl]-1-[2-(morpholin-4- |
| | yl)pyrimidin-5-yl]-2,3,4,9-tetrahydro-1H-pyrido[3,4-b]indole; |
| 467 | (1S)-6-chloro-1-(4-methylphenyl)-2-[4-methyl-6-(trifluoromethyl)-1,3,5-triazin-2-yl]- |
| | 2,3,4,9-tetrahydro-1H-pyrido[3,4-b]indole; |
| 468 | 6-chloro-1-(4-chloro-2-fluorophenyl)-2-(4,6-dimethyl-1,3,5-triazin-2-yl)-2,3,4,9- |
| | tetrahydro-1H-pyrido[3,4-b]indole; |
| 469 | 6-chloro-1-(2,3-difluoro-4-methylphenyl)-2-(4-phenylpyrimidin-2-yl)-2,3,4,9- |
| | tetrahydro-1H-pyrido[3,4-b]indole; |
| 470 | (1S)-2-(5-bromopyrimidin-2-yl)-6-chloro-1-[2-(morpholin-4-yl)pyrimidin-5-yl]- |
| | 2,3,4,9-tetrahydro-1H-pyrido[3,4-b]indole; |
| 471 | (1S)-6-chloro-1-(2,3-difluoro-4-methylphenyl)-2-(5-phenylpyrimidin-2-yl)-2,3,4,9- |
| | tetrahydro-1H-pyrido[3,4-b]indole; |
| 472 | (1S)-6-chloro-1-[2-(morpholin-4-yl)pyrimidin-5-yl]-2-(5-phenylpyrimidin-2-yl)- |
| | 2,3,4,9-tetrahydro-1H-pyrido[3,4-b]indole; |
| 473 | (1S)-6-chloro-1-[6-(morpholin-4-yl)pyridin-3-yl]-2-[4-(trifluoromethyl)pyrimidin-2- |
| | yl]-2,3,4,9-tetrahydro-1H-pyrido[3,4-b]indole; |
| 474 | (1S)-6-chloro-1-[2-(4-methylpiperazin-1-yl)pyrimidin-5-yl]-2-[4-(methylsulfanyl)-6- |
| | (trifluoromethyl)-1,3,5-triazin-2-yl]-2,3,4,9-tetrahydro-1H-pyrido[3,4-b]indole; |
| 475 | 6-chloro-1-[2-(morpholin-4-yl)pyrimidin-5-yl]-2-(4-phenylpyrimidin-2-yl)-2,3,4,9- |
| | tetrahydro-1H-pyrido[3,4-b]indole; |
| 476 | (1S)-6-chloro-1-(2,3-difluoro-4-methylphenyl)-2-[4-fluoro-6-(4-methylpiperazin-1- |
| | yl)-1,3,5-triazin-2-yl]-2,3,4,9-tetrahydro-1H-pyrido[3,4-b]indole; |
| 477 | (1S)-6-chloro-1-(2,3-difluoro-4-methylphenyl)-2-[4-(4-methylpiperazin-1-yl)-6- |
| | (trifluoromethyl)-1,3,5-triazin-2-yl]-2,3,4,9-tetrahydro-1H-pyrido[3,4-b]indole; |
| 478 | (1S)-6-chloro-2-[4-(4-methylpiperazin-1-yl)-6-(trifluoromethyl)-1,3,5-triazin-2-yl]-1- |
| | [2-(morpholin-4-yl)pyrimidin-5-yl]-2,3,4,9-tetrahydro-1H-pyrido[3,4-b]indole; |
| 479 | (1S)-6-chloro-1-[2-(4-methylpiperazin-1-yl)pyrimidin-5-yl]-2-[4-(4-methylpiperazin- |
| | 1-yl)-6-(trifluoromethyl)-1,3,5-triazin-2-yl]-2,3,4,9-tetrahydro-1H-pyrido[3,4- |
| | b]indole; |
| 480 | (1S)-6-chloro-1-[6-(4-methylpiperazin-1-yl)pyridin-3-yl]-2-[4- |
| | (trifluoromethyl)pyrimidin-2-yl]-2,3,4,9-tetrahydro-1H-pyrido[3,4-b]indole; |
| 481 | (1S)-2-[4,6-bis(trifluoromethyl)pyrimidin-2-yl]-6-chloro-1-[6-(4-methylpiperazin-1- |
| | yl)pyridin-3-yl]-2,3,4,9-tetrahydro-1H-pyrido[3,4-b]indole; |
| 482 | 2-[4,6-bis(trifluoromethyl)pyrimidin-2-yl]-1-[2-(morpholin-4-yl)pyrimidin-5-yl]-6- |
| | (2H-1,2,3-triazol-2-yl)-2,3,4,9-tetrahydro-1H-pyrido[3,4-b]indole; |
| 483 | 2-[4,6-bis(trifluoromethyl)pyrimidin-2-yl]-6-(4-methyl-2H-1,2,3-triazol-2-yl)-1-[2- |
| | (morpholin-4-yl)pyrimidin-5-yl]-2,3,4,9-tetrahydro-1H-pyrido[3,4-b]indole; |
| 484 | (1S)-6-chloro-1-[2-(4-methylpiperazin-1-yl)pyrimidin-5-yl]-2-[4- |
| | (trifluoromethyl)pyrimidin-2-yl]-2,3,4,9-tetrahydro-1H-pyrido[3,4-b]indole; |
| 485 | (1S)-2-[4,6-bis(trifluoromethyl)pyrimidin-2-yl]-1-[6-(morpholin-4-yl)pyridin-3-yl]-6- |
| | (2H-1,2,3-triazol-2-yl)-2,3,4,9-tetrahydro-1H-pyrido[3,4-b]indole; |
| 486 | (1S)-2-[4,6-bis(trifluoromethyl)-1,3,5-triazin-2-yl]-1-[6-(morpholin-4-yl)pyridin-3- |
| | yl]-6-(2H-1,2,3-triazol-2-yl)-2,3,4,9-tetrahydro-1H-pyrido[3,4-b]indole; |
| 487 | (1S)-2-[4,6-bis(trifluoromethyl)pyrimidin-2-yl]-6-bromo-1-[6-(morpholin-4- |
| | yl)pyridin-3-yl]-2,3,4,9-tetrahydro-1H-pyrido[3,4-b]indole; |
| 488 | (1S)-6-chloro-1-[2-(4-methylpiperazin-1-yl)pyrimidin-5-yl]-2-[4-(trifluoromethyl)- |
| | 1,3,5-triazin-2-yl]-2,3,4,9-tetrahydro-1H-pyrido[3,4-b]indole; |
| 489 | (1S)-6-chloro-1-[6-(4-methylpiperazin-1-yl)pyridin-3-yl]-2-[4-(methylsulfanyl)-6- |
| | (trifluoromethyl)-1,3,5-triazin-2-yl]-2,3,4,9-tetrahydro-1H-pyrido[3,4-b]indole; |
| 490 | 2-[4,6-bis(trifluoromethyl)pyrimidin-2-yl]-1-(2,3-difluoro-4-methylphenyl)-6-fluoro- |
| | 2,3,4,9-tetrahydro-1H-pyrido[3,4-b]indole; |
| ¹491 | (1S)-6-chloro-1-[6-(4-methylpiperazin-1-yl)pyridin-3-yl]-2-[4-(trifluoromethyl)-1,3,5- |
| | triazin-2-yl]-2,3,4,9-tetrahydro-1H-pyrido[3,4-b]indole; |
| 492 | 2-[4,6-bis(trifluoromethyl)-1,3,5-triazin-2-yl]-1-(2,3-difluoro-4-methylphenyl)-6- |
| | fluoro-2,3,4,9-tetrahydro-1H-pyrido[3,4-b]indole; |
| 493 | 1-(2,3-difluoro-4-methylphenyl)-6-fluoro-2-[4-(trifluoromethyl)pyrimidin-2-yl]- |
| | 2,3,4,9-tetrahydro-1H-pyrido[3,4-b]indole; |
| 494 | 1-(2,3-difluoro-4-methylphenyl)-6-fluoro-2-[4-(trifluoromethyl)-1,3,5-triazin-2-yl]- |
| | 2,3,4,9-tetrahydro-1H-pyrido[3,4-b]indole; |
| 495 | 1-(2,3-difluoro-4-methylphenyl)-6-fluoro-2-[4-(methylsulfanyl)-6-(trifluoromethyl)- |
| | 1,3,5-triazin-2-yl]-2,3,4,9-tetrahydro-1H-pyrido[3,4-b]indole; |
| 496 | (1S)-2-[4,6-bis(trifluoromethyl)pyrimidin-2-yl]-6-chloro-1-[2-(3,3-dimethylpiperazin- |
| | 1-yl)pyrimidin-5-yl]-2,3,4,9-tetrahydro-1H-pyrido[3,4-b]indole; |
| 497 | 2-[4,6-bis(trifluoromethyl)-1,3,5-triazin-2-yl]-6-chloro-1-(2,3-difluoro-4- |
| | methylphenyl)-2,3,4,9-tetrahydro-1H-pyrido[3,4-b]indole; |
| 498 | (1S)-1-[6-(morpholin-4-yl)pyridin-3-yl]-6-(2H-1,2,3-triazol-2-yl)-2-[4- |
| | (trifluoromethyl)-1,3,5-triazin-2-yl]-2,3,4,9-tetrahydro-1H-pyrido[3,4-b]indole; |
| 499 | (1S)-2-[4,6-bis(trifluoromethyl)pyrimidin-2-yl]-6-bromo-1-[2-(4-methylpiperazin-1- |
| | yl)pyrimidin-5-yl]-2,3,4,9-tetrahydro-1H-pyrido[3,4-b]indole; |
| 500 | (1S)-2-[4,6-bis(trifluoromethyl)pyrimidin-2-yl]-1-[2-(4-methylpiperazin-1- |
| | yl)pyrimidin-5-yl]-6-(2H-1,2,3-triazol-2-yl)-2,3,4,9-tetrahydro-1H-pyrido[3,4- |
| | b]indole; |
| 501 | (1S)-2-[4,6-bis(trifluoromethyl)-1,3,5-triazin-2-yl]-1-[2-(4-methylpiperazin-1- |
| | yl)pyrimidin-5-yl]-6-(2H-1,2,3-triazol-2-yl)-2,3,4,9-tetrahydro-1H-pyrido[3,4- |
| | b]indole; |
| 502 | 1-(2,3-difluoro-4-methylphenyl)-6-(2H-1,2,3-triazol-2-yl)-2-[4- |
| | (trifluoromethyl)pyrimidin-2-yl]-2,3,4,9-tetrahydro-1H-pyrido[3,4-b]indole; |
| 503 | (1S)-2-[4,6-bis(trifluoromethyl)pyrimidin-2-yl]-6-chloro-1-{2-[(3R,5S)-3,5- |
| | dimethylpiperazin-1-yl]pyrimidin-5-yl}-2,3,4,9-tetrahydro-1H-pyrido[3,4-b]indole; |
| 504 | methyl 4-[(1S)-6-chloro-1-(2,3-difluoro-4-methylphenyl)-1,3,4,9-tetrahydro-2H- |
| | pyrido[3,4-b]indol-2-yl]-6-(trifluoromethyl)-1,3,5-triazine-2-carboxylate; |
| 505 | (1S)-6-chloro-1-(2,3-difluoro-4-methylphenyl)-2-[4-(trichloromethyl)-6- |
| | (trifluoromethyl)-1,3,5-triazin-2-yl]-2,3,4,9-tetrahydro-1H-pyrido[3,4-b]indole; |
| 506 | (1S)-1-[2-(4-methylpiperazin-1-yl)pyrimidin-5-yl]-6-(2H-1,2,3-triazol-2-yl)-2-[4- |
| | (trifluoromethyl)-1,3,5-triazin-2-yl]-2,3,4,9-tetrahydro-1H-pyrido[3,4-b]indole; |
| 507 | (1S)-6-chloro-2-[4-(difluoromethyl)-6-(trifluoromethyl)-1,3,5-triazin-2-yl]-1-[2- |
| | (morpholin-4-yl)pyrimidin-5-yl]-2,3,4,9-tetrahydro-1H-pyrido[3,4-b]indole; |
| 508 | (1S)-6-chloro-2-[4-(difluoromethyl)-6-(trifluoromethyl)-1,3,5-triazin-2-yl]-1-[2-(4- |
| | methylpiperazin-1-yl)pyrimidin-5-yl]-2,3,4,9-tetrahydro-1H-pyrido[3,4-b]indole; |
| 509 | (1S)-6-chloro-1-[2-(4-methylpiperazin-1-yl)pyrimidin-5-yl]-2-[4-(morpholin-4-yl)-6- |
| | (trifluoromethyl)-1,3,5-triazin-2-yl]-2,3,4,9-tetrahydro-1H-pyrido[3,4-b]indole; |
| 510 | (1S)-6-chloro-1-(2,3-difluoro-4-methylphenyl)-2-[4-(difluoromethyl)-6- |
| | (trifluoromethyl)-1,3,5-triazin-2-yl]-2,3,4,9-tetrahydro-1H-pyrido[3,4-b]indole; |
| 511 | (1S)-2-[4,6-bis(trifluoromethyl)pyrimidin-2-yl]-6-chloro-1-{2-[(3R)-3- |
| | methylpiperazin-1-yl]pyrimidin-5-yl}-2,3,4,9-tetrahydro-1H-pyrido[3,4-b]indole; |
| 512 | (1S)-2-[4,6-bis(trifluoromethyl)pyrimidin-2-yl]-6-chloro-1-[2-(4,7- |
| | diazaspiro[2.5]octan-7-yl)pyrimidin-5-yl]-2,3,4,9-tetrahydro-1H-pyrido[3,4-b]indole; |
| 513 | 1-[6-(4-methylpiperazin-1-yl)pyridin-3-yl]-6-(2H-1,2,3-triazol-2-yl)-2-[4- |
| | (trifluoromethyl)pyrimidin-2-yl]-2,3,4,9-tetrahydro-1H-pyrido[3,4-b]indole; |
| 514 | (1S)-2-[4,6-bis(trifluoromethyl)pyrimidin-2-yl]-1-[2-(morpholin-4-yl)pyrimidin-5-yl]- |
| | 6-(2H-1,2,3-triazol-2-yl)-2,3,4,9-tetrahydro-1H-pyrido[3,4-b]indole; |
| 515 | (1S)-1-[2-(morpholin-4-yl)pyrimidin-5-yl]-6-(2H-1,2,3-triazol-2-yl)-2-[4- |
| | (trifluoromethyl)-1,3,5-triazin-2-yl]-2,3,4,9-tetrahydro-1H-pyrido[3,4-b]indole; |
| 516 | (1S)-2-[4,6-bis(trifluoromethyl)-1,3,5-triazin-2-yl]-1-[2-(morpholin-4-yl)pyrimidin-5- |
| | yl]-6-(2H-1,2,3-triazol-2-yl)-2,3,4,9-tetrahydro-1H-pyrido[3,4-b]indole; |
| 517 | (1S)-2-[4,6-bis(trifluoromethyl)pyrimidin-2-yl]-6-chloro-1-{2-[(3S)-3- |
| | methylpiperazin-1-yl]pyrimidin-5-yl}-2,3,4,9-tetrahydro-1H-pyrido[3,4-b]indole; |
| 518 | (1S)-2-[4,6-bis(trifluoromethyl)pyrimidin-2-yl]-6-bromo-1-(2,3-difluoro-4- |
| | methylphenyl)-2,3,4,9-tetrahydro-1H-pyrido[3,4-b]indole; |
| 519 | (1S)-2-[4,6-bis(trifluoromethyl)pyrimidin-2-yl]-1-(2,3-difluoro-4-methylphenyl)-6- |
| | (2H-1,2,3-triazol-2-yl)-2,3,4,9-tetrahydro-1H-pyrido[3,4-b]indole; |
| 520 | (1S)-2-[4,6-bis(trifluoromethyl)pyrimidin-2-yl]-1-(2,3-difluoro-4-methylphenyl)-6-(4- |
| | methyl-2H-1,2,3-triazol-2-yl)-2,3,4,9-tetrahydro-1H-pyrido[3,4-b]indole; |
| 521 | {4-[(1S)-6-chloro-1-(2,3-difluoro-4-methylphenyl)-1,3,4,9-tetrahydro-2H-pyrido[3,4- |
| | b]indol-2-yl]-6-(trifluoromethyl)-1,3,5-triazin-2-yl}methanol; |
| 522 | (1S)-2-[4,6-bis(trifluoromethyl)-1,3,5-triazin-2-yl]-6-chloro-1-(2-methoxypyrimidin- |
| | 5-yl)-2,3,4,9-tetrahydro-1H-pyrido[3,4-b]indole; |
| 523 | (1S)-2-[4,6-bis(trifluoromethyl)-1,3,5-triazin-2-yl]-6-bromo-1-(2,3-difluoro-4- |
| | methylphenyl)-2,3,4,9-tetrahydro-1H-pyrido[3,4-b]indole; |
| 524 | (1S)-2-[4,6-bis(trifluoromethyl)-1,3,5-triazin-2-yl]-1-(2,3-difluoro-4-methylphenyl)-6- |
| | (2H-1,2,3-triazol-2-yl)-2,3,4,9-tetrahydro-1H-pyrido[3,4-b]indole; |
| 525 | (1S)-2-[4,6-bis(trifluoromethyl)-1,3,5-triazin-2-yl]-1-(2,3-difluoro-4-methylphenyl)-6- |
| | (4-methyl-2H-1,2,3-triazol-2-yl)-2,3,4,9-tetrahydro-1H-pyrido[3,4-b]indole; |
| 526 | (1S)-6-chloro-1-(2,3-difluoro-4-methylphenyl)-2-[4-(fluoromethyl)-6- |
| | (trifluoromethyl)-1,3,5-triazin-2-yl]-2,3,4,9-tetrahydro-1H-pyrido[3,4-b]indole; |
| 527 | (1S)-2-[4,6-bis(trifluoromethyl)-1,3,5-triazin-2-yl]-1-(2,3-difluoro-4-methylphenyl)-6- |
| | nitro-2,3,4,9-tetrahydro-1H-pyrido[3,4-b]indole; |
| 528 | (1S)-2-[4,6-bis(trifluoromethyl)pyrimidin-2-yl]-1-(2,3-difluoro-4-methylphenyl)-6- |
| | methoxy-2,3,4,9-tetrahydro-1H-pyrido[3,4-b]indole; |
| 529 | (1S)-2-[4,6-bis(trifluoromethyl)-1,3,5-triazin-2-yl]-1-(2,3-difluoro-4-methylphenyl)-6- |
| | (4-methyl-1H-pyrazol-1-yl)-2,3,4,9-tetrahydro-1H-pyrido[3,4-b]indole; |
| 530 | (1S)-2-[4,6-bis(trifluoromethyl)-1,3,5-triazin-2-yl]-1-(2,3-difluoro-4-methylphenyl)-6- |
| | (3-methyl-1H-pyrazol-1-yl)-2,3,4,9-tetrahydro-1H-pyrido[3,4-b]indole; |
| 531 | N²-(5-{(1S)-2-[4,6-bis(trifluoromethyl)-1,3,5-triazin-2-yl]-6-chloro-2,3,4,9- |
| | tetrahydro-1H-pyrido[3,4-b]indol-1-yl}pyrimidin-2-yl)-N¹,N¹-dimethylethane-1,2- |
| | diamine; |
| 532 | methyl 5-{(1S)-2-[4,6-bis(trifluoromethyl)-1,3,5-triazin-2-yl]-6-chloro-2,3,4,9- |
| | tetrahydro-1H-pyrido[3,4-b]indol-1-yl}pyrimidine-2-carboxylate; |
| 533 | 1-(5-{(1S)-2-[4,6-bis(trifluoromethyl)-1,3,5-triazin-2-yl]-6-chloro-2,3,4,9-tetrahydro- |
| | 1H-pyrido[3,4-b]indol-1-yl}pyrimidin-2-yl)ethan-1-one; |
| 534 | (1S)-2-[4,6-bis(trifluoromethyl)-1,3,5-triazin-2-yl]-6-chloro-1-[2-(2- |
| | methoxyethoxy)pyrimidin-5-yl]-2,3,4,9-tetrahydro-1H-pyrido[3,4-b]indole; |
| 535 | 2-[(5-{(1S)-2-[4,6-bis(trifluoromethyl)-1,3,5-triazin-2-yl]-6-chloro-2,3,4,9-tetrahydro- |
| | 1H-pyrido[3,4-b]indol-1-yl}pyrimidin-2-yl)amino]ethan-1-ol; |
| 536 | 2-[4,6-bis(trifluoromethyl)-1,3,5-triazin-2-yl]-6-chloro-1-[2-(2- |
| | methoxyethoxy)pyrimidin-5-yl]-2,3,4,9-tetrahydro-1H-pyrido[3,4-b]indole; |
| 537 | N-(5-{(1S)-2-[4,6-bis(trifluoromethyl)-1,3,5-triazin-2-yl]-6-chloro-2,3,4,9-tetrahydro- |
| | 1H-pyrido[3,4-b]indol-1-yl}pyrimidin-2-yl)-B-alanine; |
| 538 | 4-[(5-{(1S)-2-[4,6-bis(trifluoromethyl)-1,3,5-triazin-2-yl]-6-chloro-2,3,4,9-tetrahydro- |
| | 1H-pyrido[3,4-b]indol-1-yl}pyrimidin-2-yl)amino]butanoic acid; and |
| 539 | (2-[(5-{(1S)-2-[4,6-bis(trifluoromethyl)-1,3,5-triazin-2-yl]-6-chloro-2,3,4,9- |
| | tetrahydro-1H-pyrido[3,4-b]indol-1-yl}pyrimidin-2-yl)amino]ethoxy}acetic acid; |

wherein the form of the compound is selected from the group consisting of a salt, hydrate, solvate, racemate, enantiomer, diastereomer, stereoisomer, and tautomer form thereof.

Another aspect of the compound of Formula (I) or a form thereof includes a compound (Cpd) salt selected from the group consisting of:

| Cpd | Name |
|---|---|
| 357 | 2-[4,6-bis(trifluoromethyl)pyrimidin-2-yl]-6-chloro-1-[6-(morpholin-4-yl)pyridin-3-yl]- |
| | 2,3,4,9-tetrahydro-1H-pyrido[3,4-b]indole formate (1:1); |
| 358 | 2-[4,6-bis(trifluoromethyl)pyrimidin-2-yl]-6-chloro-1-[2-(morpholin-4-yl)pyrimidin-5- |
| | yl]-2,3,4,9-tetrahydro-1H-pyrido[3,4-b]indole formate (1:1); and, |
| 491 | (1S)-6-chloro-1-[6-(4-methylpiperazin-1-yl)pyridin-3-yl]-2-[4-(trifluoromethyl)-1,3,5- |
| | triazin-2-yl]-2,3,4,9-tetrahydro-1H-py.rido[3,4-b]indole formate (1:1); |

wherein the form of the compound is selected from the group consisting of a hydrate, solvate, racemate, enantiomer, diastereomer, stereoisomer, and tautomer form thereof.

One aspect provided herein is a pharmaceutical composition comprising a compound of Formula (I) or a form thereof, and a pharmaceutically acceptable excipient.

One aspect provided herein is a use of a compound of Formula (I), or a form thereof, or pharmaceutical composition thereof, to treat or ameliorate a disease or disorder by inhibiting dihydroorotate dehydrogenase.

Another aspect provided herein is a use of a compound of Formula (I), or a form thereof, to treat or ameliorate a disease or disorder by inhibiting dihydroorotate dehydrogenase.

Another aspect provided herein is a use of a pharmaceutical composition comprising a compound of Formula (I), or a form thereof, and a pharmaceutically acceptable excipient to treat or ameliorate a disease or disorder by inhibiting dihydroorotate dehydrogenase.

One aspect provided herein is a method of use of a compound of Formula (I), or a form thereof, or pharmaceutical composition thereof to treat or ameliorate a disease or disorder in a subject in need thereof comprising, administering to the subject an effective amount of the compound of Formula (I), or a form thereof, or pharmaceutical composition thereof to inhibit dihydroorotate dehydrogenase.

Another aspect provided herein is a method of use of a compound of Formula (I), or a form thereof, to treat or ameliorate a disease or disorder in a subject in need thereof comprising, administering to the subject an effective amount of a compound of Formula (I), or a form thereof, to inhibit dihydroorotate dehydrogenase.

Another aspect provided herein is a method of use of a pharmaceutical composition comprising a compound of Formula (I), or a form thereof, and a pharmaceutically acceptable excipient to treat or ameliorate a disease or disorder in a subject in need thereof comprising, administering to the subject an effective amount of the pharmaceutical composition to inhibit dihydroorotate dehydrogenase.

An aspect of the compound of Formula (I) or a form thereof includes a compound (Cpd) having dihydroorotate dehydrogenase inhibition activity ≤10.0 nM selected from the group consisting of (wherein ¹Cpd #indicates the compound was isolated as a salt form):

| Cpd | Name |
|---|---|
| 7 | 6-chloro-2-(4,6-dichloropyrimidin-2-yl)-1-(4-methylphenyl)-2,3,4,9-tetrahydro-1H- |
| | pyrido[3,4-b]indole; |
| 10 | 6-chloro-2-(4-chloropyrimidin-2-yl)-1-(4-methylphenyl)-2,3,4,9-tetrahydro-1H- |
| | pyrido[3,4-b]indole; |
| 16 | 6-chloro-2-(4,6-dichloropyrimidin-2-yl)-7-fluoro-1-(4-methylphenyl)-2,3,4,9-tetrahydro- |
| | 1H-pyrido[3,4-b]indole; |
| 21 | 6-chloro-2-(5-chloropyrimidin-2-yl)-5-fluoro-1-(4-methylphenyl)-2,3,4,9-tetrahydro-1H- |
| | pyrido[3,4-b]indole; |
| 28 | 6-chloro-2-(4-chloropyrimidin-2-yl)-5-fluoro-1-(4-methylphenyl)-2,3,4,9-tetrahydro-1H- |
| | pyrido[3,4-b]indole; |
| 43 | 6-chloro-2-(4-chloropyrimidin-2-yl)-7-fluoro-1-(4-methylphenyl)-2,3,4,9-tetrahydro-1H- |
| | pyrido[3,4-b]indole; |
| 46 | 6-chloro-2-(4-chloro-6-methylpyrimidin-2-yl)-7-fluoro-1-(4-fluorophenyl)-2,3,4,9- |
| | tetrahydro-1H-pyrido[3,4-b]indole; |
| 53 | 6-chloro-2-(4-chloropyrimidin-2-yl)-7-fluoro-1-[4-(propan-2-yl)phenyl]-2,3,4,9- |
| | tetrahydro-1H-pyrido[3,4-b]indole; |
| 60 | 6-chloro-5-fluoro-1-(4-fluorophenyl)-2-[4-(trifluoromethyl)pyrimidin-2-yl]-2,3,4,9- |
| | tetrahydro-1H-pyrido[3,4-b]indole; |
| 66 | 6-chloro-2-(4,6-dimethylpyrimidin-2-yl)-7-fluoro-1-(4-methylphenyl)-2,3,4,9-tetrahydro- |
| | 1H-pyrido[3,4-b]indole; |
| 68 | 2-[6-chloro-1-(2,3-difluorophenyl)-7-fluoro-1,3,4,9-tetrahydro-2H-pyrido[3,4-b]indol-2- |
| | yl]pyrimidine-4-carbonitrile; |
| 70 | 6-chloro-1-(4-chlorophenyl)-5-fluoro-2-(5-fluoro-4-methylpyrimidin-2-yl)-2,3,4,9- |
| | tetrahydro-1H-pyrido[3,4-b]indole; |
| 72 | 6-chloro-7-fluoro-1-(4-methylphenyl)-2-[4-(trifluoromethyl)pyrimidin-2-yl]-2,3,4,9- |
| | tetrahydro-1H-pyrido[3,4-b]indole; |
| 73 | 6-chloro-1-(4-chlorophenyl)-5-fluoro-2-[4-(trifluoromethyl)pyrimidin-2-yl]-2,3,4,9- |
| | tetrahydro-1H-pyrido[3,4-b]indole; |
| 74 | 6-chloro-5-fluoro-1-[4-(trifluoromethyl)phenyl]-2-[4-(trifluoromethyl)pyrimidin-2-yl]- |
| | 2,3,4,9-tetrahydro-1H-pyrido[3,4-b]indole; |
| 76 | 6-chloro-5-fluoro-1-(4-methylphenyl)-2-[4-(trifluoromethyl)pyrimidin-2-yl]-2,3,4,9- |
| | tetrahydro-1H-pyrido[3,4-b]indole; |
| 77 | 6-chloro-2-(4-chloropyrimidin-2-yl)-1-(2,3-difluorophenyl)-7-fluoro-2,3,4,9-tetrahydro- |
| | 1H-pyrido[3,4-b]indole; |
| 87 | methyl 2-[6-chloro-1-(4-chlorophenyl)-5-fluoro-1,3,4,9-tetrahydro-2H-pyrido[3,4- |
| | b]indol-2-yl]pyrimidine-4-carboxylate; |
| 88 | 4-{6-chloro-7-fluoro-2-[4-methyl-6-(trifluoromethyl)pyrimidin-2-yl]-2,3,4,9-tetrahydro- |
| | 1H-pyrido[3,4-b]indol-1-yl}benzonitrile; |
| 89 | 4-{6-chloro-5-fluoro-2-[4-(trifluoromethyl)pyrimidin-2-yl]-2,3,4,9-tetrahydro-1H- |
| | pyrido[3,4-b]indol-1-yl}benzonitrile; |
| 93 | 4-[6-chloro-2-(4,6-dimethylpyrimidin-2-yl)-7-fluoro-2,3,4,9-tetrahydro-1H-pyrido[3,4- |
| | blindol-1-yl]benzonitrile; |
| 95 | 5-{6-chloro-7-fluoro-2-[4-(trifluoromethyl)pyrimidin-2-yl]-2,3,4,9-tetrahydro-1H- |
| | pyrido[3,4-b]indol-1-yl}-N,N-dimethylpyrimidin-2-amine; |
| 96 | 6-chloro-7-fluoro-1-(4-methylphenyl)-2-[4-methyl-6-(trifluoromethyl)pyrimidin-2-yl]- |
| | 2,3,4,9-tetrahydro-1H-pyrido[3,4-b]indole; |
| 100 | 4-{6-chloro-7-fluoro-2-[4-(trifluoromethyl)pyrimidin-2-yl]-2,3,4,9-tetrahydro-1H- |
| | pyrido[3,4-b]indol-1-yl}benzonitrile; |
| 106 | 6-chloro-7-fluoro-2-(5-fluoro-4,6-dimethylpyrimidin-2-yl)-1-(4-methylphenyl)-2,3,4,9- |
| | tetrahydro-1H-pyrido[3,4-b]indole; |
| 108 | 6-chloro-1-(2,3-difluorophenyl)-7-fluoro-2-[4-(trifluoromethyl)pyrimidin-2-yl]-2,3,4,9- |
| | tetrahydro-1H-pyrido[3,4-b]indole; |
| 109 | 6-chloro-5-fluoro-2-(5-fluoro-4-methylpyrimidin-2-yl)-1-(4-methylphenyl)-2,3,4,9- |
| | tetrahydro-1H-pyrido[3,4-b]indole; |
| 110 | 2-[6-chloro-5-fluoro-1-(4-methylphenyl)-1,3,4,9-tetrahydro-2H-pyrido[3,4-b]indol-2-yl]- |
| | 6-methylpyrimidine-4-carbonitrile; |
| 111 | 2-[6-chloro-5-fluoro-1-(4-fluorophenyl)-1,3,4,9-tetrahydro-2H-pyrido[3,4-b]indol-2-yl]- |
| | 6-methylpyrimidine-4-carbonitrile; |
| 112 | 6-chloro-1-(2,3-difluorophenyl)-7-fluoro-2-(5-fluoro-4,6-dimethylpyrimidin-2-yl)- |
| | 2,3,4,9-tetrahydro-1H-pyrido[3,4-b]indole; |
| 113 | 6-chloro-1-(2,3-difluorophenyl)-5-fluoro-2-(5-fluoro-4-methylpyrimidin-2-yl)-2,3,4,9- |
| | tetrahydro-1H-pyrido[3,4-b]indole; |
| 114 | 6-chloro-1-(2,3-difluoro-4-methylphenyl)-5-fluoro-2-(5-fluoro-4-methylpyrimidin-2-yl)- |
| | 2,3,4,9-tetrahydro-1H-pyrido[3,4-b]indole; |
| 115 | 4-{6-chloro-7-fluoro-2-[4-(trifluoromethyl)pyrimidin-2-yl]-2,3,4,9-tetrahydro-1H- |
| | pyrido[3,4-b]indol-1-yl}benzene-1-sulfonamide; |
| 116 | 6-chloro-5-fluoro-2-(5-fluoro-4,6-dimethylpyrimidin-2-yl)-1-(4-methylphenyl)-2,3,4,9- |
| | tetrahydro-1H-pyrido[3,4-b]indole; |
| 117 | 6-chloro-1-(2,3-difluorophenyl)-5-fluoro-2-(5-fluoro-4,6-dimethylpyrimidin-2-yl)- |
| | 2,3,4,9-tetrahydro-1H-pyrido[3,4-b]indole; |
| 118 | 6-chloro-1-(2,3-difluoro-4-methylphenyl)-5-fluoro-2-(5-fluoro-4,6-dimethylpyrimidin-2- |
| | yl)-2,3,4,9-tetrahydro-1H-pyrido[3,4-b]indole; |
| 119 | 6-chloro-1-(2,3-difluorophenyl)-2-[4-(trifluoromethyl)pyrimidin-2-yl]-2,3,4,9-tetrahydro- |
| | 1H-pyrido[3,4-b]indole; |
| 120 | 6-chloro-1-(2,3-difluoro-4-methoxyphenyl)-2-[4-(trifluoromethyl)pyrimidin-2-yl]- |
| | 2,3,4,9-tetrahydro-1H-pyrido[3,4-b]indole; |
| 122 | 5-{6-chloro-7-fluoro-2-[4-(trifluoromethyl)pyrimidin-2-yl]-2,3,4,9-tetrahydro-1H- |
| | pyrido[3,4-b]indol-1-yl}-N,N-dimethylpyridin-2-amine; |
| 124 | 6-chloro-1-(2,3-difluorophenyl)-7-fluoro-2-(5-fluoro-4-methylpyrimidin-2-yl)-2,3,4,9- |
| | tetrahydro-1H-pyrido[3,4-b]indole; |
| 125 | 6-chloro-1-(2,3-difluoro-4-methylphenyl)-7-fluoro-2-(5-fluoro-4-methylpyrimidin-2-yl)- |
| | 2,3,4,9-tetrahydro-1H-pyrido[3,4-b]indole; |
| 128 | 6-chloro-1-(2,3-difluoro-4-methylphenyl)-2-[4-(trifluoromethyl)pyrimidin-2-yl]-2,3,4,9- |
| | tetrahydro-1H-pyrido[3,4-b]indole; |
| 129 | 6-chloro-1-(2,3-difluorophenyl)-2-(4,6-dimethylpyrimidin-2-yl)-7-fluoro-2,3,4,9- |
| | tetrahydro-1H-pyrido[3,4-b]indole; |
| 130 | 6-chloro-2-(5-chloropyrimidin-2-yl)-1-(2,3-difluoro-4-methylphenyl)-7-fluoro-2,3,4,9- |
| | tetrahydro-1H-pyrido[3,4-b]indole; |
| 131 | 6-chloro-2-(5-chloropyrimidin-2-yl)-1-(2,3-difluorophenyl)-7-fluoro-2,3,4,9-tetrahydro- |
| | 1H-pyrido[3,4-b]indole; |
| 132 | 6-chloro-1-(2,3-difluoro-4-methylphenyl)-2-(4,6-dimethylpyrimidin-2-yl)-7-fluoro- |
| | 2,3,4,9-tetrahydro-1H-pyrido[3,4-b]indole; |
| 136 | 6-chloro-1-(2,3-difluoro-4-methylphenyl)-7-fluoro-2-(5-methylpyrimidin-2-yl)-2,3,4,9- |
| | tetrahydro-1H-pyrido[3,4-b]indole; |
| 137 | 6-chloro-1-(2,3-difluoro-4-methylphenyl)-5-fluoro-2-(5-methylpyrimidin-2-yl)-2,3,4,9- |
| | tetrahydro-1H-pyrido[3,4-b]indole; |
| 138 | 6-chloro-1-(2,3-difluoro-4-methylphenyl)-7-fluoro-2-(5-fluoropyrimidin-2-yl)-2,3,4,9- |
| | tetrahydro-1H-pyrido[3,4-b]indole; |
| 139 | 6-chloro-1-(2,3-difluoro-4-methylphenyl)-7-fluoro-2-(4-methylpyrimidin-2-yl)-2,3,4,9- |
| | tetrahydro-1H-pyrido[3,4-b]indole; |
| 140 | 6-chloro-1-(2,3-difluoro-4-methylphenyl)-7-fluoro-2-[4-(trifluoromethyl)pyrimidin-2-yl]- |
| | 2,3,4,9-tetrahydro-1H-pyrido[3,4-b]indole; |
| 141 | 2-[6-chloro-1-(2,3-difluoro-4-methylphenyl)-7-fluoro-1,3,4,9-tetrahydro-2H-pyrido[3,4- |
| | b]indol-2-yl]pyrimidine-4-carbonitrile; |
| 142 | 6-chloro-1-(2,3-difluoro-4-methylphenyl)-7-fluoro-2-[5-(trifluoromethyl)pyrimidin-2-yl]- |
| | 2,3,4,9-tetrahydro-1H-pyrido[3,4-b]indole; |
| 143 | 6-chloro-1-(2,3-difluoro-4-methylphenyl)-7-fluoro-2-(5-fluoro-4,6-dimethylpyrimidin-2- |
| | yl)-2,3,4,9-tetrahydro-1H-pyrido[3,4-b]indole; |
| 144 | 6-chloro-1-(4-chloro-2,3-difluorophenyl)-2-[4-(trifluoromethyl)pyrimidin-2-yl]-2,3,4,9- |
| | tetrahydro-1H-pyrido[3,4-b]indole; |
| 147 | 6-chloro-1-(2,3-difluorophenyl)-7-fluoro-2-[4-methyl-6-(trifluoromethyl)pyrimidin-2-yl]- |
| | 2,3,4,9-tetrahydro-1H-pyrido[3,4-b]indole; |
| 148 | 6-chloro-1-(2,3-difluoro-4-methylphenyl)-7-fluoro-2-[4-methyl-6- |
| | (trifluoromethyl)pyrimidin-2-yl]-2,3,4,9-tetrahydro-1H-pyrido[3,4-b]indole; |
| 149 | 6-chloro-5-fluoro-1-[4-(methanesulfonyl)phenyl]-2-[4-(trifluoromethyl)pyrimidin-2-yl]- |
| | 2,3,4,9-tetrahydro-1H-pyrido[3,4-b]indole; |
| 150 | 4-{6-chloro-5-fluoro-2-[4-(trifluoromethyl)pyrimidin-2-yl]-2,3,4,9-tetrahydro-1H- |
| | pyrido[3,4-b]indol-1-yl}benzene-1-sulfonamide; |
| 151 | 2-[6-chloro-7-fluoro-1-(4-methylphenyl)-1,3,4,9-tetrahydro-2H-pyrido[3,4-b]indol-2-yl]- |
| | 6-methylpyrimidine-4-carbonitrile; |
| 152 | 2-[6-chloro-1-(2,3-difluorophenyl)-7-fluoro-1,3,4,9-tetrahydro-2H-pyrido[3,4-b]indol-2- |
| | yl]-6-methylpyrimidine-4-carbonitrile; |
| 153 | 2-[6-chloro-1-(2,3-difluoro-4-methylphenyl)-7-fluoro-1,3,4,9-tetrahydro-2H-pyrido[3,4- |
| | blindol-2-yl]-6-methylpyrimidine-4-carbonitrile; |
| 154 | 2-[4,6-bis(trifluoromethyl)pyrimidin-2-yl]-6-chloro-7-fluoro-1-(4-methylphenyl)-2,3,4,9- |
| | tetrahydro-1H-pyrido[3,4-b]indole; |
| 155 | 2-[4,6-bis(trifluoromethyl)pyrimidin-2-yl]-6-chloro-1-(2,3-difluoro-4-methylphenyl)-7- |
| | fluoro-2,3,4,9-tetrahydro-1H-pyrido[3,4-b]indole; |
| 156 | 3-(4-{6-chloro-7-fluoro-2-[4-(trifluoromethyl)pyrimidin-2-yl]-2,3,4,9-tetrahydro-1H- |
| | pyrido[3,4-b]indol-1-yl}phenyl)propan-1-ol; |
| 157 | 2-[4,6-bis(trifluoromethyl)pyrimidin-2-yl]-6-chloro-1-(2,3-difluorophenyl)-7-fluoro- |
| | 2,3,4,9-tetrahydro-1H-pyrido[3,4-b]indole; |
| 158 | 5-{6-chloro-7-fluoro-2-[4-(trifluoromethyl)pyrimidin-2-yl]-2,3,4,9-tetrahydro-1H- |
| | pyrido[3,4-b]indol-1-yl}-3-fluoro-N,N-dimethylpyridin-2-amine; |
| 160 | 5-{6-chloro-5-fluoro-2-[4-(trifluoromethyl)pyrimidin-2-yl]-2,3,4,9-tetrahydro-1H- |
| | pyrido[3,4-b]indol-1-yl}-3-fluoro-N,N-dimethylpyridin-2-amine; |
| 161 | (1S)-6-chloro-1-(4-methylphenyl)-2-[4-(trifluoromethyl)pyrimidin-2-yl]-2,3,4,9- |
| | tetrahydro-1H-pyrido[3,4-b]indole; |
| 162 | 2-[6-chloro-1-(2,3-difluoro-4-methylphenyl)-7-fluoro-1,3,4,9-tetrahydro-2H-pyrido[3,4- |
| | b]indol-2-yl]-5-fluoropyrimidine-4-carbonitrile; |
| 164 | methyl 3-(4-{6-chloro-7-fluoro-2-[4-(trifluoromethyl)pyrimidin-2-yl]-2,3,4,9-tetrahydro- |
| | 1H-pyrido[3,4-b]indol-1-yl}phenyl)propanoate; |
| 166 | (4-{6-chloro-7-fluoro-2-[4-(trifluoromethyl)pyrimidin-2-yl]-2,3,4,9-tetrahydro-1H- |
| | pyrido[3,4-b]indol-1-yl}phenyl)methanol; |
| 167 | (3E)-4-(4-{6-chloro-7-fluoro-2-[4-(trifluoromethyl)pyrimidin-2-yl]-2,3,4,9-tetrahydro- |
| | 1H-pyrido[3,4-b]indol-1-yl}phenyl)but-3-en-1-ol; |
| 168 | 4-(4-{6-chloro-7-fluoro-2-[4-(trifluoromethyl)pyrimidin-2-yl]-2,3,4,9-tetrahydro-1H- |
| | pyrido[3,4-b]indol-1-yl}phenyl)butan-1-ol; |
| 169 | 6-chloro-1-(2,3-difluoro-4-methylphenyl)-7-fluoro-2-(4,5,6-trimethylpyrimidin-2-yl)- |
| | 2,3,4,9-tetrahydro-1H-pyrido[3,4-b]indole; |
| 171 | 6-chloro-1-(2,3-difluorophenyl)-7-fluoro-2-(4,5,6-trimethylpyrimidin-2-yl)-2,3,4,9- |
| | tetrahydro-1H-pyrido[3,4-b]indole; |
| 172 | 1-(4-bromo-2,3-difluorophenyl)-6-chloro-7-fluoro-2-[4-(trifluoromethyl)pyrimidin-2-yl]- |
| | 2,3,4,9-tetrahydro-1H-pyrido[3,4-b]indole; |
| 173 | 4-{6-chloro-7-fluoro-2-[4-(trifluoromethyl)pyrimidin-2-yl]-2,3,4,9-tetrahydro-1H- |
| | pyrido[3,4-b]indol-1-yl}-2,3-difluoro-N,N-dimethylaniline; |
| 174 | 5-fluoro-6-methyl-1-(4-methylphenyl)-2-[4-(trifluoromethyl)pyrimidin-2-yl]-2,3,4,9- |
| | tetrahydro-1H-pyrido[3,4-b]indole; |
| 175 | 2-[6-chloro-7-fluoro-1-(4-methylphenyl)-1,3,4,9-tetrahydro-2H-pyrido[3,4-b]indol-2-yl]- |
| | 5-fluoropyrimidine-4-carbonitrile; |
| 177 | 5-{6-chloro-5-fluoro-2-[4-(trifluoromethyl)pyrimidin-2-yl]-2,3,4,9-tetrahydro-1H- |
| | pyrido[3,4-b]indol-1-yl}-N,N-dimethylpyridin-2-amine; |
| 178 | 5-{6-chloro-5-fluoro-2-[4-(trifluoromethyl)pyrimidin-2-yl]-2,3,4,9-tetrahydro-1H- |
| | pyrido[3,4-b]indol-1-yl}-N,N-dimethylpyrimidin-2-amine; |
| 182 | (1S)-6-chloro-1-(4-chlorophenyl)-5-fluoro-2-[4-(trifluoromethyl)pyrimidin-2-yl]-2,3,4,9- |
| | tetrahydro-1H-pyrido[3,4-b]indole; |
| 183 | 2-[6-chloro-1-(2,3-difluorophenyl)-7-fluoro-1,3,4,9-tetrahydro-2H-pyrido[3,4-b]indol-2- |
| | yl]-5-fluoropyrimidine-4-carbonitrile; |
| 184 | 6-chloro-1-(2,6-difluoropyridin-3-yl)-7-fluoro-2-[4-(trifluoromethyl)pyrimidin-2-yl]- |
| | 2,3,4,9-tetrahydro-1H-pyrido[3,4-b]indole; |
| 186 | 6-chloro-1-(2,6-difluoropyridin-3-yl)-5-fluoro-2-[4-(trifluoromethyl)pyrimidin-2-yl]- |
| | 2,3,4,9-tetrahydro-1H-pyrido[3,4-b]indole; |
| 188 | 4-{2-[4,6-bis(trifluoromethyl)pyrimidin-2-yl]-6-chloro-7-fluoro-2,3,4,9-tetrahydro-1H- |
| | pyrido[3,4-b]indol-1-yl}benzonitrile; |
| 189 | (1S)-6-chloro-1-(2,3-difluoro-4-methylphenyl)-5-fluoro-2-(5-fluoro-4,6- |
| | dimethylpyrimidin-2-yl)-2,3,4,9-tetrahydro-1H-pyrido[3,4-b]indole; |
| 192 | 1-(2,3-difluoro-4-methylphenyl)-5-fluoro-6-methyl-2-[4-(trifluoromethyl)pyrimidin-2- |
| | yl]-2,3,4,9-tetrahydro-1H-pyrido[3,4-b]indole; |
| 193 | 6-chloro-5-fluoro-1-[2-(1H-imidazol-1-yl)pyrimidin-5-yl]-2-[4- |
| | (trifluoromethyl)pyrimidin-2-yl]-2,3,4,9-tetrahydro-1H-pyrido[3,4-b]indole; |
| 195 | 6-chloro-2-(5-chloropyrimidin-2-yl)-1-(2,3-difluoro-4-methylphenyl)-5-fluoro-2,3,4,9- |
| | tetrahydro-1H-pyrido[3,4-b]indole; |
| 196 | 6-chloro-1-(2,3-difluoro-4-methylphenyl)-5-fluoro-2-(4-methylpyrimidin-2-yl)-2,3,4,9- |
| | tetrahydro-1H-pyrido[3,4-b]indole; |
| 197 | 6-chloro-1-(2,3-difluoro-4-methylphenyl)-5-fluoro-2-[4-(trifluoromethyl)pyrimidin-2-yl]- |
| | 2,3,4,9-tetrahydro-1H-pyrido[3,4-b]indole; |
| 198 | 2-[6-chloro-1-(2,3-difluoro-4-methylphenyl)-5-fluoro-1,3,4,9-tetrahydro-2H-pyrido[3,4- |
| | b]indol-2-yl]pyrimidine-4-carbonitrile; |
| 199 | 5-{6-chloro-5-fluoro-2-[4-(trifluoromethyl)pyrimidin-2-yl]-2,3,4,9-tetrahydro-1H- |
| | pyrido[3,4-b]indol-1-yl}-N-methylpyrimidin-2-amine; |
| 200 | 6-chloro-5-fluoro-1-[2-(morpholin-4-yl)pyrimidin-5-yl]-2-[4-(trifluoromethyl)pyrimidin- |
| | 2-yl]-2,3,4,9-tetrahydro-1H-pyrido[3,4-b]indole; |
| 201 | 5-[6-chloro-5-fluoro-2-(5-fluoropyrimidin-2-yl)-2,3,4,9-tetrahydro-1H-pyrido[3,4- |
| | b]indol-1-yl]-N,N-dimethylpyrimidin-2-amine; |
| 203 | 6-chloro-1-(2,3-difluoro-4-methylphenyl)-2-(4-methylpyrimidin-2-yl)-2,3,4,9-tetrahydro- |
| | 1H-pyrido[3,4-b]indole; |
| 205 | 6-chloro-1-(2,3-difluoro-4-methylphenyl)-2-(4,6-dimethylpyrimidin-2-yl)-5-fluoro- |
| | 2,3,4,9-tetrahydro-1H-pyrido[3,4-b]indole; |
| 206 | 6-chloro-1-(2,3-difluoro-4-methylphenyl)-5-fluoro-2-[4-methyl-6- |
| | (trifluoromethyl)pyrimidin-2-yl]-2,3,4,9-tetrahydro-1H-pyrido[3,4-b]indole; |
| 207 | 6-chloro-1-(4-methylphenyl)-2-[4-(trifluoromethyl)pyrimidin-2-yl]-2,3,4,9-tetrahydro- |
| | 1H-pyrido[3,4-b]indol-8-ol; |
| 208 | 5-{6-chloro-7-fluoro-2-[4-(trifluoromethyl)pyrimidin-2-yl]-2,3,4,9-tetrahydro-1H- |
| | pyrido[3,4-b]indol-1-yl}-6-fluoro-N,N-dimethylpyridin-2-amine; |
| 209 | 2-[4,6-bis(trifluoromethyl)pyrimidin-2-yl]-6-chloro-1-(2,3-difluoro-4-methylphenyl)-5- |
| | fluoro-2,3,4,9-tetrahydro-1H-pyrido[3,4-b]indole; |
| 210 | 5-{6-chloro-5-fluoro-2-[4-(trifluoromethyl)pyrimidin-2-yl]-2,3,4,9-tetrahydro-1H- |
| | pyrido[3,4-b]indol-1-yl}-6-fluoro-N,N-dimethylpyridin-2-amine; |
| 211 | 6-chloro-5-fluoro-2-(5-fluoropyrimidin-2-yl)-1-[2-(morpholin-4-yl)pyrimidin-5-yl]- |
| | 2,3,4,9-tetrahydro-1H-pyrido[3,4-b]indole; |
| 214 | 2-[6-chloro-1-(2,3-difluoro-4-methylphenyl)-5-fluoro-1,3,4,9-tetrahydro-2H-pyrido[3,4- |
| | b]indol-2-yl]-5-fluoropyrimidine-4-carbonitrile; |
| 215 | 2-[6-chloro-7-fluoro-1-(4-methylphenyl)-1,3,4,9-tetrahydro-2H-pyrido[3,4-b]indol-2- |
| | yl]pyrimidine-4,6-dicarbonitrile; |
| 216 | 2-[6-chloro-1-(2,3-difluoro-4-methylphenyl)-7-fluoro-1,3,4,9-tetrahydro-2H-pyrido[3,4- |
| | b]indol-2-yl]pyrimidine-4,6-dicarbonitrile; |
| 217 | 2-[6-chloro-5-fluoro-1-(4-methylphenyl)-1,3,4,9-tetrahydro-2H-pyrido[3,4-b]indol-2-yl]- |
| | 5-fluoropyrimidine-4-carbonitrile; |
| 218 | 2-chloro-5-{6-chloro-2-[4-(trifluoromethyl)pyrimidin-2-yl]-2,3,4,9-tetrahydro-1H- |
| | pyrido[3,4-b]indol-1-yl}phenol; |
| 220 | 6-chloro-1-(2,3-difluoro-4-methylphenyl)-2-[4-(trifluoromethyl)pyrimidin-2-yl]-2,3,4,9- |
| | tetrahydro-1H-pyrido[3,4-b]indol-8-ol; |
| 221 | 5-chloro-2-[6-chloro-2-(5-fluoropyrimidin-2-yl)-2,3,4,9-tetrahydro-1H-pyrido[3,4- |
| | b]indol-1-yl]phenol; |
| 222 | 5-chloro-2-{6-chloro-2-[4-(trifluoromethyl)pyrimidin-2-yl]-2,3,4,9-tetrahydro-1H- |
| | pyrido[3,4-b]indol-1-yl}phenol; |
| 223 | 2-[(4-{6-chloro-7-fluoro-2-[4-(trifluoromethyl)pyrimidin-2-yl]-2,3,4,9-tetrahydro-1H- |
| | pyrido[3,4-b]indol-1-yl}phenyl)methoxy]ethan-1-ol; |
| 224 | 2-(6-chloro-7-fluoro-1-{4-[(1E)-4-hydroxybut-1-en-1-yl]phenyl}-1,3,4,9-tetrahydro-2H- |
| | pyrido[3,4-b]indol-2-yl)pyrimidine-4-carbonitrile; |
| 228 | 2-[6-chloro-5-fluoro-1-(4-methylphenyl)-1,3,4,9-tetrahydro-2H-pyrido[3,4-b]indol-2- |
| | yl]pyrimidine-4,6-dicarbonitrile; |
| 229 | 2-[6-chloro-1-(2,3-difluoro-4-methylphenyl)-5-fluoro-1,3,4,9-tetrahydro-2H-pyrido[3,4- |
| | b]indol-2-yl]pyrimidine-4,6-dicarbonitrile; |
| 230 | 6-chloro-5-fluoro-1-[2-(1H-1,2,4-triazol-1-yl)pyrimidin-5-yl]-2-[4- |
| | (trifluoromethyl)pyrimidin-2-yl]-2,3,4,9-tetrahydro-1H-pyrido[3,4-b]indole; |
| 231 | 6-chloro-1-(4-methylphenyl)-2-[4-(trifluoromethyl)pyrimidin-2-yl]-2,3,4,9-tetrahydro- |
| | 1H-pyrido[3,4-b]indol-8-amine; |
| 232 | 2-[6-chloro-1-(2,3-difluoro-4-methylphenyl)-5-fluoro-1,3,4,9-tetrahydro-2H-pyrido[3,4- |
| | b]indol-2-yl]-6-methylpyrimidine-4-carbonitrile; |
| 233 | (1S)-6-chloro-1-(2,3-difluoro-4-methylphenyl)-2-[4-(trifluoromethyl)pyrimidin-2-yl]- |
| | 2,3,4,9-tetrahydro-1H-pyrido[3,4-b]indole; |
| 234 | (1S)-6-chloro-1-(2,3-difluoro-4-methylphenyl)-2-(4-methylpyrimidin-2-yl)-2,3,4,9- |
| | tetrahydro-1H-pyrido[3,4-b]indole; |
| 235 | 6-chloro-1-(2,3-difluoro-4-methylphenyl)-2-(5-fluoro-4-methylpyrimidin-2-yl)-2,3,4,9- |
| | tetrahydro-1H-pyrido[3,4-b]indole; |
| 236 | 2-(6-chloro-5-fluoro-1-{4-[(1E)-4-hydroxybut-1-en-1-yl]phenyl}-1,3,4,9-tetrahydro-2H- |
| | pyrido[3,4-b]indol-2-yl)pyrimidine-4-carbonitrile; |
| 237 | (3E)-4-{4-[6-chloro-5-fluoro-2-(4-methylpyrimidin-2-yl)-2,3,4,9-tetrahydro-1H- |
| | pyrido[3,4-b]indol-1-yl]phenyl}but-3-en-1-ol; |
| 238 | (1S)-6-chloro-1-(2,3-difluoro-4-methylphenyl)-2-(5-fluoropyrimidin-2-yl)-2,3,4,9- |
| | tetrahydro-1H-pyrido[3,4-b]indole; |
| 239 | (3E)-4-{4-[6-chloro-7-fluoro-2-(5-fluoro-4-methylpyrimidin-2-yl)-2,3,4,9-tetrahydro-1H- |
| | pyrido[3,4-b]indol-1-yl]phenyl}but-3-en-1-ol; |
| 240 | 4-{4-[6-chloro-7-fluoro-2-(5-fluoro-4-methylpyrimidin-2-yl)-2,3,4,9-tetrahydro-1H- |
| | pyrido[3,4-b]indol-1-yl]phenyl}butan-1-ol; |
| 241 | (3E)-4-{4-[6-chloro-5-fluoro-2-(5-fluoro-4-methylpyrimidin-2-yl)-2,3,4,9-tetrahydro-1H- |
| | pyrido[3,4-b]indol-1-yl]phenyl}but-3-en-1-ol; |
| 242 | (3E)-4-{4-[6-chloro-2-(4,6-dimethylpyrimidin-2-yl)-5-fluoro-2,3,4,9-tetrahydro-1H- |
| | pyrido[3,4-b]indol-1-yl]phenyl}but-3-en-1-ol; |
| 243 | (3E)-4-{4-[6-chloro-5-fluoro-2-(5-fluoro-4,6-dimethylpyrimidin-2-yl)-2,3,4,9-tetrahydro- |
| | 1H-pyrido[3,4-b]indol-1-yl]phenyl}but-3-en-1-ol; |
| 244 | (3E)-4-{4-[6-chloro-2-(4,6-dimethylpyrimidin-2-yl)-7-fluoro-2,3,4,9-tetrahydro-1H- |
| | pyrido[3,4-b]indol-1-yl]phenyl}but-3-en-1-ol; |
| 245 | 4-{4-[6-chloro-2-(4,6-dimethylpyrimidin-2-yl)-7-fluoro-2,3,4,9-tetrahydro-1H- |
| | pyrido[3,4-b]indol-1-yl]phenyl}butan-1-ol; |
| 246 | 4-{4-[6-chloro-5-fluoro-2-(5-fluoro-4-methylpyrimidin-2-yl)-2,3,4,9-tetrahydro-1H- |
| | pyrido[3,4-b]indol-1-yl]phenyl}butan-1-ol; |
| 247 | 5-bromo-6-chloro-1-(4-chlorophenyl)-2-[4-(trifluoromethyl)pyrimidin-2-yl]-2,3,4,9- |
| | tetrahydro-1H-pyrido[3,4-b]indole; |
| 248 | 6-chloro-1-(2,3-difluoro-4-methylphenyl)-2-[4-(trifluoromethyl)pyrimidin-2-yl]-2,3,4,9- |
| | tetrahydro-1H-pyrido[3,4-b]indol-8-amine; |
| 249 | 4-{4-[6-chloro-7-fluoro-2-(5-fluoro-4,6-dimethylpyrimidin-2-yl)-2,3,4,9-tetrahydro-1H- |
| | pyrido[3,4-b]indol-1-yl]phenyl}butan-1-ol; |
| 251 | 6-chloro-1-[2-(morpholin-4-yl)pyrimidin-5-yl]-2-[4-(trifluoromethyl)pyrimidin-2-yl]- |
| | 2,3,4,9-tetrahydro-1H-pyrido[3,4-b]indole; |
| 252 | 2-{6-chloro-2-[4-(trifluoromethyl)pyrimidin-2-yl]-2,3,4,9-tetrahydro-1H-pyrido[3,4- |
| | b]indol-1-yl}-6-fluorophenol; |
| 253 | 6-chloro-1-(2,3-difluoro-4-methylphenyl)-2-(4,6-dimethyl-1,3,5-triazin-2-yl)-2,3,4,9- |
| | tetrahydro-1H-pyrido[3,4-b]indole; |
| 254 | 6-chloro-1-(2-fluoro-4-methylphenyl)-2-[4-(trifluoromethyl)pyrimidin-2-yl]-2,3,4,9- |
| | tetrahydro-1H-pyrido[3,4-b]indole; |
| 255 | 4-{6-chloro-2-[4-(trifluoromethyl)pyrimidin-2-yl]-2,3,4,9-tetrahydro-1H-pyrido[3,4- |
| | b]indol-1-yl}-3-fluorobenzonitrile; |
| 256 | 3-{6-chloro-2-[4-(trifluoromethyl)pyrimidin-2-yl]-2,3,4,9-tetrahydro-1H-pyrido[3,4- |
| | b]indol-1-yl}-2-fluorophenol; |
| 257 | 2-{6-chloro-2-[4-(trifluoromethyl)pyrimidin-2-yl]-2,3,4,9-tetrahydro-1H-pyrido[3,4- |
| | b]indol-1-yl}-5-methylphenol; |
| 259 | 6-chloro-1-(2-fluoro-3-methylphenyl)-2-[4-(trifluoromethyl)pyrimidin-2-yl]-2,3,4,9- |
| | tetrahydro-1H-pyrido[3,4-b]indole; |
| 260 | 6-chloro-1-[4-(1H-imidazol-1-yl)phenyl]-2-[4-(trifluoromethyl)pyrimidin-2-yl]-2,3,4,9- |
| | tetrahydro-1H-pyrido[3,4-b]indole; |
| 261 | 6-chloro-1-[4-(1H-pyrazol-1-yl)phenyl]-2-[4-(trifluoromethyl)pyrimidin-2-yl]-2,3,4,9- |
| | tetrahydro-1H-pyrido[3,4-b]indole; |
| 262 | 6-chloro-1-[4-(morpholin-4-yl)phenyl]-2-[4-(trifluoromethyl)pyrimidin-2-yl]-2,3,4,9- |
| | tetrahydro-1H-pyrido[3,4-b]indole; |
| 263 | 6-chloro-2-[4-(trifluoromethyl)pyrimidin-2-yl]-1-(2,3,4-trifluorophenyl)-2,3,4,9- |
| | tetrahydro-1H-pyrido[3,4-b]indole; |
| 265 | 1-(4-methylphenyl)-6-nitro-2-[4-(trifluoromethyl)pyrimidin-2-yl]-2,3,4,9-tetrahydro-1H- |
| | pyrido[3,4-b]indole; |
| 266 | 6-chloro-1-(2,3-difluoro-4-methylphenyl)-5-nitro-2-[4-(trifluoromethyl)pyrimidin-2-yl]- |
| | 2,3,4,9-tetrahydro-1H-pyrido[3,4-b]indole; |
| 267 | 6-chloro-1-(4-chloro-2-fluorophenyl)-2-[4-(trifluoromethyl)pyrimidin-2-yl]-2,3,4,9- |
| | tetrahydro-1H-pyrido[3,4-b]indole; |
| 268 | 1-(4-bromo-2-fluorophenyl)-6-chloro-2-[4-(trifluoromethyl)pyrimidin-2-yl]-2,3,4,9- |
| | tetrahydro-1H-pyrido[3,4-b]indole; |
| 270 | 1-(4-bromo-2,3-difluorophenyl)-6-chloro-2-[4-(trifluoromethyl)pyrimidin-2-yl]-2,3,4,9- |
| | tetrahydro-1H-pyrido[3,4-b]indole; |
| 271 | 1-(2,3-difluoro-4-methylphenyl)-6-methyl-2-[4-(trifluoromethyl)pyrimidin-2-yl]-2,3,4,9- |
| | tetrahydro-1H-pyrido[3,4-b]indole; |
| 272 | 1-(2,3-difluoro-4-methylphenyl)-6-methyl-2-[4-(trifluoromethyl)pyrimidin-2-yl]-2,3,4,9- |
| | tetrahydro-1H-pyrido[3,4-b]indol-8-amine; |
| 273 | 6-chloro-1-(2,3-difluoro-4-methylphenyl)-2-[4-(trifluoromethyl)pyrimidin-2-yl]-2,3,4,9- |
| | tetrahydro-1H-pyrido[3,4-b]indol-5-amine; |
| 275 | 6-chloro-1-[2-fluoro-4-(1H-imidazol-1-yl)phenyl]-2-[4-(trifluoromethyl)pyrimidin-2-yl]- |
| | 2,3,4,9-tetrahydro-1H-pyrido[3,4-b]indole; |
| 276 | 1-(2,3-difluoro-4-methylphenyl)-6-methyl-2-[4-(trifluoromethyl)pyrimidin-2-yl]-2,3,4,9- |
| | tetrahydro-1H-pyrido[3,4-b]indol-8-ol; |
| 277 | 6-chloro-1-[2-fluoro-4-(morpholin-4-yl)phenyl]-2-[4-(trifluoromethyl)pyrimidin-2-yl]- |
| | 2,3,4,9-tetrahydro-1H-pyrido[3,4-b]indole; |
| 278 | 4-{6-chloro-2-[4-(trifluoromethyl)pyrimidin-2-yl]-2,3,4,9-tetrahydro-1H-pyrido[3,4- |
| | b]indol-1-yl}-2,3-difluorobenzonitrile; |
| 279 | 6-chloro-1-[2,3-difluoro-4-(morpholin-4-yl)phenyl]-2-[4-(trifluoromethyl)pyrimidin-2- |
| | yl]-2,3,4,9-tetrahydro-1H-pyrido[3,4-b]indole; |
| 280 | butyl 2-[6-chloro-1-(4-methylphenyl)-1,3,4,9-tetrahydro-2H-pyrido[3,4-b]indol-2- |
| | yl]pyrimidine-4-carboxylate; |
| 281 | butyl 2-[6-chloro-1-(2,3-difluoro-4-methylphenyl)-1,3,4,9-tetrahydro-2H-pyrido[3,4- |
| | b]indol-2-yl]pyrimidine-4-carboxylate; |
| 283 | 1-(4-bromo-2,3-difluorophenyl)-6-chloro-2-(4,6-dimethyl-1,3,5-triazin-2-yl)-2,3,4,9- |
| | tetrahydro-1H-pyrido[3,4-b]indole; |
| 285 | 6-chloro-1-[2,3-difluoro-4-(1H-imidazol-1-yl)phenyl]-2-[4-(trifluoromethyl)pyrimidin-2- |
| | yl]-2,3,4,9-tetrahydro-1H-pyrido[3,4-b]indole; |
| 286 | 6-chloro-2-(4,6-dimethylpyrimidin-2-yl)-1-(4-methylphenyl)-2,3,4,9-tetrahydro-1H- |
| | pyrido[3,4-b]indole; |
| 287 | 1-(4-methylphenyl)-6-(2H-1,2,3-triazol-2-yl)-2-[4-(trifluoromethyl)pyrimidin-2-yl]- |
| | 2,3,4,9-tetrahydro-1H-pyrido[3,4-b]indole; |
| 288 | 1-[2-(morpholin-4-yl)pyrimidin-5-yl]-6-(2H-1,2,3-triazol-2-yl)-2-[4- |
| | (trifluoromethyl)pyrimidin-2-yl]-2,3,4,9-tetrahydro-1H-pyrido[3,4-b]indole; |
| 291 | 6-ethenyl-1-(4-methylphenyl)-2-[4-(trifluoromethyl)pyrimidin-2-yl]-2,3,4,9-tetrahydro- |
| | 1H-pyrido[3,4-b]indole; |
| 298 | 6-bromo-1-[2-(morpholin-4-yl)pyrimidin-5-yl]-2-[4-(trifluoromethyl)pyrimidin-2-yl]- |
| | 2,3,4,9-tetrahydro-1H-pyrido[3,4-b]indole; |
| 299 | 6-bromo-2-(4,6-dimethyl-1,3,5-triazin-2-yl)-1-[2-(morpholin-4-yl)pyrimidin-5-yl]- |
| | 2,3,4,9-tetrahydro-1H-pyrido[3,4-b]indole; |
| 302 | 6-methoxy-1-[2-(morpholin-4-yl)pyrimidin-5-yl]-2-[4-(trifluoromethyl)pyrimidin-2-yl]- |
| | 2,3,4,9-tetrahydro-1H-pyrido[3,4-b]indole; |
| 303 | 6-chloro-1-[6-(morpholin-4-yl)pyridin-3-yl]-2-[4-(trifluoromethyl)pyrimidin-2-yl]- |
| | 2,3,4,9-tetrahydro-1H-pyrido[3,4-b]indole; |
| 305 | 1-(2,3-difluoro-4-methylphenyl)-6-nitro-2-[4-(trifluoromethyl)pyrimidin-2-yl]-2,3,4,9- |
| | tetrahydro-1H-pyrido[3,4-b]indole; |
| 308 | 6-chloro-2-(4,6-dimethylpyrimidin-2-yl)-1-[2-(morpholin-4-yl)pyrimidin-5-yl]-2,3,4,9- |
| | tetrahydro-1H-pyrido[3,4-b]indole; |
| 309 | 6-chloro-2-(4,6-dimethyl-1,3,5-triazin-2-yl)-1-[2-(morpholin-4-yl)pyrimidin-5-yl]- |
| | 2,3,4,9-tetrahydro-1H-pyrido[3,4-b]indol-8-amino; |
| 310 | 6-chloro-1-[2-(morpholin-4-yl)pyrimidin-5-yl]-2-[4-(trifluoromethyl)pyrimidin-2-yl]- |
| | 2,3,4,9-tetrahydro-1H-pyrido[3,4-b]indol-8-amine; |
| 311 | 6-chloro-1-[2-(morpholin-4-yl)pyrimidin-5-yl]-2-[4-(trifluoromethyl)pyrimidin-2-yl]- |
| | 2,3,4,9-tetrahydro-1H-pyrido[3,4-b]indol-7-amine; |
| 312 | 6-chloro-2-(4-methylpyrimidin-2-yl)-1-[2-(morpholin-4-yl)pyrimidin-5-yl]-2,3,4,9- |
| | tetrahydro-1H-pyrido[3,4-b]indole; |
| 316 | 1-[6-(morpholin-4-yl)pyridin-3-yl]-6-(2H-1,2,3-triazol-2-yl)-2-[4- |
| | (trifluoromethyl)pyrimidin-2-yl]-2,3,4,9-tetrahydro-1H-pyrido[3,4-b]indole; |
| 317 | N-butyl-4-{6-chloro-1-[2-(morpholin-4-yl)pyrimidin-5-yl]-1,3,4,9-tetrahydro-2H- |
| | pyrido[3,4-b]indol-2-yl}-N,6-dimethyl-1,3,5-triazin-2-amine; |
| 318 | 2-(4-butoxy-6-methyl-1,3,5-triazin-2-yl)-6-chloro-1-[2-(morpholin-4-yl)pyrimidin-5-yl]- |
| | 2,3,4,9-tetrahydro-1H-pyrido[3,4-b]indole; |
| 321 | 6-chloro-2-(4,6-dimethyl-1,3,5-triazin-2-yl)-1-[6-(morpholin-4-yl)pyridin-3-yl]-2,3,4,9- |
| | tetrahydro-1H-pyrido[3,4-b]indol-8-amine; |
| 323 | 1-[2-(morpholin-4-yl)pyrimidin-5-yl]-6-nitro-2-[4-(trifluoromethyl)pyrimidin-2-yl]- |
| | 2,3,4,9-tetrahydro-1H-pyrido[3,4-b]indole; |
| 328 | 6-chloro-1-[2-(morpholin-4-yl)pyrimidin-5-yl]-2-[4-(trifluoromethyl)pyrimidin-2-yl]- |
| | 2,3,4,9-tetrahydro-1H-pyrido[3,4-b]indol-5-amine; |
| 335 | 6-chloro-1-[2-(2-oxa-5-azabicyclo[2.2.1]heptan-5-yl)pyrimidin-5-yl]-2-[4- |
| | (trifluoromethyl)pyrimidin-2-yl]-2,3,4,9-tetrahydro-1H-pyrido[3,4-b]indole; |
| 337 | 6-chloro-1-[2-(3-oxa-8-azabicyclo[3.2.1]octan-8-yl)pyrimidin-5-yl]-2-[4- |
| | (trifluoromethyl)pyrimidin-2-yl]-2,3,4,9-tetrahydro-1H-pyrido[3,4-b]indole; |
| 340 | 2-[4,6-bis(trifluoromethyl)pyrimidin-2-yl]-6-chloro-1-(2,3-difluoro-4-methylphenyl)- |
| | 2,3,4,9-tetrahydro-1H-pyrido[3,4-b]indole; |
| 341 | 6-chloro-1-(2-fluorophenyl)-2-[4-(trifluoromethyl)pyrimidin-2-yl]-2,3,4,9-tetrahydro-1H- |
| | pyrido[3,4-b]indole; |
| 342 | 6-chloro-1-(2,3-difluoro-4-methylphenyl)-2-(4,6-dimethylpyrimidin-2-yl)-2,3,4,9- |
| | tetrahydro-1H-pyrido[3,4-b]indole; |
| 343 | 6-chloro-1-(4-methylphenyl)-2-[4-(trifluoromethyl)pyrimidin-2-yl]-2,3,4,9-tetrahydro- |
| | 1H-pyrido[3,4-b]indole; |
| 346 | 6-chloro-2-(4,6-dimethyl-1,3,5-triazin-2-yl)-1-[2-(methylsulfanyl)pyrimidin-5-yl]- |
| | 2,3,4,9-tetrahydro-1H-pyrido[3,4-b]indole; |
| 347 | (1S)-6-chloro-1-(2,3-difluoro-4-methylphenyl)-2-(4,6-dimethyl-1,3,5-triazin-2-yl)- |
| | 2,3,4,9-tetrahydro-1H-pyrido[3,4-b]indole; |
| 348 | 6-chloro-1-(2,3-difluoro-4-methylphenyl)-2-(4,6-dimethyl-1,3,5-triazin-2-yl)-2,3,4,9- |
| | tetrahydro-1H-pyrido[3,4-b]indol-8-ol; |
| 349 | 6-chloro-1-(4-chlorophenyl)-2-[4-(trifluoromethyl)pyrimidin-2-yl]-2,3,4,9-tetrahydro-1H- |
| | pyrido[3,4-b]indole; |
| 350 | 6-chloro-2-(4,6-dimethylpyrimidin-2-yl)-1-[6-(morpholin-4-yl)pyridin-3-yl]-2,3,4,9- |
| | tetrahydro-1H-pyrido[3,4-b]indole; |
| 351 | 6-chloro-2-(4-methylpyrimidin-2-yl)-1-[6-(morpholin-4-yl)pyridin-3-yl]-2,3,4,9- |
| | tetrahydro-1H-pyrido[3,4-b]indol-8-amine; |
| 352 | 6-chloro-2-(4-methylpyrimidin-2-yl)-1-[6-(morpholin-4-yl)pyridin-3-yl]-2,3,4,9- |
| | tetrahydro-1H-pyrido[3,4-b]indole; |
| 353 | 2-{6-chloro-2-[4-(trifluoromethyl)pyrimidin-2-yl]-2,3,4,9-tetrahydro-1H-pyrido[3,4- |
| | b]indol-1-yl}phenol; |
| 354 | 6-chloro-1-(3-fluoro-4-methylphenyl)-2-[4-(trifluoromethyl)pyrimidin-2-yl]-2,3,4,9- |
| | tetrahydro-1H-pyrido[3,4-b]indole; |
| 356 | 2-{6-chloro-1-[6-(morpholin-4-yl)pyridin-3-yl]-1,3,4,9-tetrahydro-2H-pyrido[3,4- |
| | b]indol-2-yl}pyrimidine-4-carbonitrile; |
| ¹357 | 2-[4,6-bis(trifluoromethyl)pyrimidin-2-yl]-6-chloro-1-[6-(morpholin-4-yl)pyridin-3-yl]- |
| | 2,3,4,9-tetrahydro-1H-pyrido[3,4-b]indole; |
| ¹358 | 2-[4,6-bis(trifluoromethyl)pyrimidin-2-yl]-6-chloro-1-[2-(morpholin-4-yl)pyrimidin-5- |
| | yl]-2,3,4,9-tetrahydro-1H-pyrido[3,4-b]indole; |
| 359 | 2-{6-chloro-1-[2-(morpholin-4-yl)pyrimidin-5-yl]-1,3,4,9-tetrahydro-2H-pyrido[3,4- |
| | b]indol-2-yl}pyrimidine-4-carbonitrile; |
| 360 | 2-[4,6-bis(trifluoromethyl)pyrimidin-2-yl]-6-chloro-1-(4-methylphenyl)-2,3,4,9- |
| | tetrahydro-1H-pyrido[3,4-b]indole; |
| 361 | 6-chloro-1-(4-methylphenyl)-2-[4-methyl-6-(trifluoromethyl)pyrimidin-2-yl]-2,3,4,9- |
| | tetrahydro-1H-pyrido[3,4-b]indole; |
| 362 | 2-{6-chloro-1-[2-(morpholin-4-yl)pyrimidin-5-yl]-1,3,4,9-tetrahydro-2H-pyrido[3,4- |
| | b]indol-2-yl}-6-methylpyrimidine-4-carbonitrile; |
| 363 | 2-[6-chloro-1-(2,3-difluoro-4-methylphenyl)-1,3,4,9-tetrahydro-2H-pyrido[3,4-b]indol-2- |
| | yl]pyrimidine-4-carbonitrile; |
| 365 | 6-chloro-2-(5-fluoro-4-methylpyrimidin-2-yl)-1-[6-(morpholin-4-yl)pyridin-3-yl]-2,3,4,9- |
| | tetrahydro-1H-pyrido[3,4-b]indole; |
| 367 | 6-chloro-2-(5-fluoro-4,6-dimethylpyrimidin-2-yl)-1-[6-(morpholin-4-yl)pyridin-3-yl]- |
| | 2,3,4,9-tetrahydro-1H-pyrido[3,4-b]indole; |
| 368 | 6-chloro-2-(5-fluoro-4,6-dimethylpyrimidin-2-yl)-1-[2-(morpholin-4-yl)pyrimidin-5-yl]- |
| | 2,3,4,9-tetrahydro-1H-pyrido[3,4-b]indole; |
| 369 | 6-chloro-1-[2-(4-methylpiperazin-1-yl)pyrimidin-5-yl]-2-[4-(trifluoromethyl)pyrimidin-2- |
| | yl]-2,3,4,9-tetrahydro-1H-pyrido[3,4-b]indole; |
| 370 | 6-chloro-2-(4,6-dimethyl-1,3,5-triazin-2-yl)-1-[2-(morpholin-4-yl)pyrimidin-5-yl]- |
| | 2,3,4,9-tetrahydro-1H-pyrido[3,4-b]indol-8-ol; |
| 371 | 6-chloro-1-(3-fluorophenyl)-2-[4-(trifluoromethyl)pyrimidin-2-yl]-2,3,4,9-tetrahydro-1H- |
| | pyrido[3,4-b]indole; |
| 372 | 6-chloro-2-[4-methyl-6-(trifluoromethyl)pyrimidin-2-yl]-1-[6-(morpholin-4-yl)pyridin-3- |
| | yl]-2,3,4,9-tetrahydro-1H-pyrido[3,4-b]indole; |
| 373 | 6-chloro-1-{6-[(1R,4R)-2-oxa-5-azabicyclo[2.2.1]heptan-5-yl]pyridin-3-yl}-2-[4- |
| | (trifluoromethyl)pyrimidin-2-yl]-2,3,4,9-tetrahydro-1H-pyrido[3,4-b]indole; |
| 374 | 6-chloro-2-[4-methyl-6-(trifluoromethyl)pyrimidin-2-yl]-1-[2-(morpholin-4-yl)pyrimidin- |
| | 5-yl]-2,3,4,9-tetrahydro-1H-pyrido[3,4-b]indole; |
| 375 | 6-chloro-1-{6-[(1S,4S)-2-oxa-5-azabicyclo[2.2.1]heptan-5-yl]pyridin-3-yl}-2-[4- |
| | (trifluoromethyl)pyrimidin-2-yl]-2,3,4,9-tetrahydro-1H-pyrido[3,4-b]indole; |
| 378 | 2-[6-chloro-1-(4-methylphenyl)-1,3,4,9-tetrahydro-2H-pyrido[3,4-b]indol-2- |
| | yl]pyrimidine-4-carbonitrile; |
| 379 | 2-[6-chloro-1-(4-methylphenyl)-1,3,4,9-tetrahydro-2H-pyrido[3,4-b]indol-2-yl]-6- |
| | methylpyrimidine-4-carbonitrile; |
| 381 | 2-[4,6-bis(trifluoromethyl)pyrimidin-2-yl]-6-chloro-1-[2-(4-methylpiperazin-1- |
| | yl)pyrimidin-5-yl]-2,3,4,9-tetrahydro-1H-pyrido[3,4-b]indole; |
| 383 | 6-chloro-1-[2-(morpholin-4-yl)pyrimidin-5-yl]-2-[5-(trifluoromethyl)-1,2,4-triazin-3-yl]- |
| | 2,3,4,9-tetrahydro-1H-pyrido[3,4-b]indole; |
| 385 | 2-[(1S)-6-chloro-1-(2,3-difluoro-4-methylphenyl)-1,3,4,9-tetrahydro-2H-pyrido[3,4- |
| | b]indol-2-yl]-5-fluoropyrimidine-4-carbonitrile; |
| 386 | (1S)-2-[4,6-bis(trifluoromethyl)-1,3,5-triazin-2-yl]-6-chloro-1-(4-methylphenyl)-2,3,4,9- |
| | tetrahydro-1H-pyrido[3,4-b]indole; |
| 387 | (1S)-2-[4,6-bis(trifluoromethyl)-1,3,5-triazin-2-yl]-6-chloro-1-(2,3-difluoro-4- |
| | methylphenyl)-2,3,4,9-tetrahydro-1H-pyrido[3,4-b]indole; |
| 388 | 2-[4,6-bis(trifluoromethyl)-1,3,5-triazin-2-yl]-6-chloro-1-[2-(morpholin-4-yl)pyrimidin- |
| | 5-yl]-2,3,4,9-tetrahydro-1H-pyrido[3,4-b]indole; |
| 389 | (1S)-6-chloro-1-(2,3-difluoro-4-methylphenyl)-2-[4-(methylsulfanyl)-6-(trifluoromethyl)- |
| | 1,3,5-triazin-2-yl]-2,3,4,9-tetrahydro-1H-pyrido[3,4-b]indole; |
| 390 | 4-[(1S)-6-chloro-1-(2,3-difluoro-4-methylphenyl)-1,3,4,9-tetrahydro-2H-pyrido[3,4- |
| | b]indol-2-yl]-6-(trifluoromethyl)-1,3,5-triazine-2-carbonitrile; |
| 391 | 6-chloro-1-[2-(2-oxa-6-azaspiro[3.3]heptan-6-yl)pyrimidin-5-yl]-2-[4- |
| | (trifluoromethyl)pyrimidin-2-yl]-2,3,4,9-tetrahydro-1H-pyrido[3,4-b]indole; |
| 392 | 2-[(1S)-6-chloro-1-(4-methylphenyl)-1,3,4,9-tetrahydro-2H-pyrido[3,4-b]indol-2-yl]-5- |
| | fluoropyrimidine-4-carbonitrile; |
| 394 | (1S)-6-chloro-2-(5-fluoro-4-methylpyrimidin-2-yl)-1-(4-methylphenyl)-2,3,4,9- |
| | tetrahydro-1H-pyrido[3,4-b]indole; |
| 395 | (1S)-6-chloro-2-(5-fluoro-4,6-dimethylpyrimidin-2-yl)-1-(4-methylphenyl)-2,3,4,9- |
| | tetrahydro-1H-pyrido[3,4-b]indole; |
| 396 | 2-{(1S)-6-chloro-1-[2-(morpholin-4-yl)pyrimidin-5-yl]-1,3,4,9-tetrahydro-2H-pyrido[3,4- |
| | b]indol-2-yl}-5-fluoropyrimidine-4-carbonitrile; |
| 397 | 2-[(1S)-6-chloro-1-(4-methylphenyl)-1,3,4,9-tetrahydro-2H-pyrido[3,4-b]indol-2- |
| | yl]pyrimidine-4,6-dicarbonitrile; |
| 400 | 2-{(1S)-6-chloro-1-[6-(morpholin-4-yl)pyridin-3-yl]-1,3,4,9-tetrahydro-2H-pyrido[3,4- |
| | b]indol-2-yl}-5-fluoropyrimidine-4-carbonitrile; |
| 401 | 6-chloro-1-(2,3-difluoro-4-methylphenyl)-2-(5-fluoropyrimidin-2-yl)-2,3,4,9-tetrahydro- |
| | 1H-pyrido[3,4-b]indole; |
| 402 | (1S)-6-chloro-2-(4,6-dimethyl-1,3,5-triazin-2-yl)-1-(4-methylphenyl)-2,3,4,9-tetrahydro- |
| | 1H-pyrido[3,4-b]indole; |
| 403 | 6-chloro-1-(2,3-difluoro-4-methylphenyl)-2-[5-(trifluoromethyl)-1,2,4-triazin-3-yl]- |
| | 2,3,4,9-tetrahydro-1H-pyrido[3,4-b]indole; |
| 404 | 2-[4,6-bis(trifluoromethyl)-1,3,5-triazin-2-yl]-6-chloro-1-[6-(morpholin-4-yl)pyridin-3- |
| | yl]-2,3,4,9-tetrahydro-1H-pyrido[3,4-b]indole; |
| 405 | (1S)-2-[4,6-bis(trifluoromethyl)-1,3,5-triazin-2-yl]-6-chloro-1-[6-(morpholin-4- |
| | yl)pyridin-3-yl]-2,3,4,9-tetrahydro-1H-pyrido[3,4-b]indole; |
| 406 | (1S)-2-[4,6-bis(trifluoromethyl)-1,3,5-triazin-2-yl]-6-chloro-1-[2-(morpholin-4- |
| | yl)pyrimidin-5-yl]-2,3,4,9-tetrahydro-1H-pyrido[3,4-b]indole; |
| 407 | (1S)-2-[4,6-bis(trifluoromethyl)pyrimidin-2-yl]-6-chloro-1-(2,3-difluoro-4- |
| | methylphenyl)-2,3,4,9-tetrahydro-1H-pyrido[3,4-b]indole; |
| 408 | (1S)-6-chloro-1-[2-(morpholin-4-yl)pyrimidin-5-yl]-2-[4-(trifluoromethyl)pyrimidin-2- |
| | yl]-2,3,4,9-tetrahydro-1H-pyrido[3,4-b]indole; |
| 409 | (1S)-2-[4,6-bis(trifluoromethyl)pyrimidin-2-yl]-6-chloro-1-[6-(morpholin-4-yl)pyridin-3- |
| | yl]-2,3,4,9-tetrahydro-1H-pyrido[3,4-b]indole; |
| 410 | (1S)-2-[4,6-bis(trifluoromethyl)pyrimidin-2-yl]-6-chloro-1-[2-(morpholin-4-yl)pyrimidin- |
| | 5-yl]-2,3,4,9-tetrahydro-1H-pyrido[3,4-b]indole; |
| 411 | 4-[(1S)-6-chloro-1-(4-methylphenyl)-1,3,4,9-tetrahydro-2H-pyrido[3,4-b]indol-2-yl]-6- |
| | methyl-1,3,5-triazine-2-carbonitrile; |
| 412 | (1S)-6-chloro-1-(2,3-difluoro-4-methylphenyl)-2-(5-fluoro-4,6-dimethylpyrimidin-2-yl)- |
| | 2,3,4,9-tetrahydro-1H-pyrido[3,4-b]indole; |
| 413 | 2-[(1S)-6-chloro-1-(2,3-difluoro-4-methylphenyl)-1,3,4,9-tetrahydro-2H-pyrido[3,4- |
| | b]indol-2-yl]-5-fluoro-6-methylpyrimidine-4-carbonitrile; |
| 416 | 2-[(1S)-6-chloro-1-(2,3-difluoro-4-methylphenyl)-1,3,4,9-tetrahydro-2H-pyrido[3,4- |
| | b]indol-2-yl]-6-(trifluoromethyl)pyrimidine-4-carbonitrile; |
| 417 | 2-[(1S)-6-chloro-1-(4-methylphenyl)-1,3,4,9-tetrahydro-2H-pyrido[3,4-b]indol-2-yl]-5- |
| | fluoro-6-methylpyrimidine-4-carbonitrile; |
| 418 | (1S)-6-chloro-1-(4-methylphenyl)-2-[4-(trifluoromethyl)-1,3,5-triazin-2-yl]-2,3,4,9- |
| | tetrahydro-1H-pyrido[3,4-b]indole; |
| 419 | (1S)-2-[4,6-bis(trifluoromethyl)pyrimidin-2-yl]-6-chloro-1-[2-(4-methylpiperazin-1- |
| | yl)pyrimidin-5-yl]-2,3,4,9-tetrahydro-1H-pyrido[3,4-b]indole; |
| 420 | (1S)-2-[4,6-bis(trifluoromethyl)-1,3,5-triazin-2-yl]-6-chloro-1-[2-(4-methylpiperazin-1- |
| | yl)pyrimidin-5-yl]-2,3,4,9-tetrahydro-1H-pyrido[3,4-b]indole; |
| 421 | 2-{(1S)-6-chloro-1-[2-(morpholin-4-yl)pyrimidin-5-yl]-1,3,4,9-tetrahydro-2H-pyrido[3,4- |
| | b]indol-2-yl}-5-fluoro-6-methylpyrimidine-4-carbonitrile; |
| 422 | 6-chloro-1-(4-fluorophenyl)-2-[4-(trifluoromethyl)pyrimidin-2-yl]-2,3,4,9-tetrahydro-1H- |
| | pyrido[3,4-b]indole; |
| 423 | 6-chloro-1-(2,4-difluorophenyl)-2-[4-(trifluoromethyl)pyrimidin-2-yl]-2,3,4,9-tetrahydro- |
| | 1H-pyrido[3,4-b]indole; |
| 424 | 2-{(1S)-6-chloro-1-[6-(morpholin-4-yl)pyridin-3-yl]-1,3,4,9-tetrahydro-2H-pyrido[3,4- |
| | b]indol-2-yl}-5-fluoro-6-methylpyrimidine-4-carbonitrile; |
| 425 | 2-[(1S)-6-chloro-1-(4-methylphenyl)-1,3,4,9-tetrahydro-2H-pyrido[3,4-b]indol-2-yl]-6- |
| | (trifluoromethyl)pyrimidine-4-carbonitrile; |
| 428 | 6-chloro-1-[6-(morpholin-4-yl)pyridin-3-yl]-2-[4-(morpholin-4-yl)-6-(trifluoromethyl)- |
| | 1,3,5-triazin-2-yl]-2,3,4,9-tetrahydro-1H-pyrido[3,4-b]indole; |
| 429 | 6-chloro-1-[6-(morpholin-4-yl)pyridin-3-yl]-2-[5-(trifluoromethyl)-1,2,4-triazin-3-yl]- |
| | 2,3,4,9-tetrahydro-1H-pyrido[3,4-b]indole; |
| 430 | 6-chloro-1-(4-methylphenyl)-2-[5-(trifluoromethyl)-1,2,4-triazin-3-yl]-2,3,4,9-tetrahydro- |
| | 1H-pyrido[3,4-b]indole; |
| 431 | 6-chloro-1-(2,3-difluoro-4-methylphenyl)-2-[6-(trifluoromethyl)-1,2,4-triazin-3-yl]- |
| | 2,3,4,9-tetrahydro-1H-pyrido[3,4-b]indole; |
| 432 | 6-chloro-1-[2-fluoro-6-(morpholin-4-yl)pyridin-3-yl]-2-[4-(trifluoromethyl)pyrimidin-2- |
| | yl]-2,3,4,9-tetrahydro-1H-pyrido[3,4-b]indole; |
| 433 | 2-{(1S)-6-chloro-1-[6-(morpholin-4-yl)pyridin-3-yl]-1,3,4,9-tetrahydro-2H-pyrido[3,4- |
| | b]indol-2-yl}-6-(trifluoromethyl)pyrimidine-4-carbonitrile; |
| 434 | 2-{(1S)-6-chloro-1-[2-(morpholin-4-yl)pyrimidin-5-yl]-1,3,4,9-tetrahydro-2H-pyrido[3,4- |
| | b]indol-2-yl}-6-(trifluoromethyl)pyrimidine-4-carbonitrile; |
| 435 | 4-[(1S)-6-chloro-1-(2,3-difluoro-4-methylphenyl)-1,3,4,9-tetrahydro-2H-pyrido[3,4- |
| | b]indol-2-yl]-N,N-dimethyl-6-(trifluoromethyl)-1,3,5-triazin-2-amine; |
| 436 | (1S)-6-chloro-1-(2,3-difluoro-4-methylphenyl)-2-[4-(morpholin-4-yl)-6-(trifluoromethyl)- |
| | 1,3,5-triazin-2-yl]-2,3,4,9-tetrahydro-1H-pyrido[3,4-b]indole; |
| 437 | 6-chloro-1-(4-methylphenyl)-2-[4-methyl-6-(trifluoromethyl)-1,3,5-triazin-2-yl]-2,3,4,9- |
| | tetrahydro-1H-pyrido[3,4-b]indole; |
| 438 | 6-chloro-1-(2,3-difluoro-4-methylphenyl)-2-[4-methyl-6-(trifluoromethyl)-1,3,5-triazin- |
| | 2-yl]-2,3,4,9-tetrahydro-1H-pyrido[3,4-b]indole; |
| 439 | 6-chloro-2-[4-methyl-6-(trifluoromethyl)-1,3,5-triazin-2-yl]-1-[6-(morpholin-4- |
| | yl)pyridin-3-yl]-2,3,4,9-tetrahydro-1H-pyrido[3,4-b]indole; |
| 440 | 6-chloro-2-[4-methyl-6-(trifluoromethyl)-1,3,5-triazin-2-yl]-1-[2-(morpholin-4- |
| | yl)pyrimidin-5-yl]-2,3,4,9-tetrahydro-1H-pyrido[3,4-b]indole; |
| 441 | 4-[(1S)-6-chloro-1-(4-methylphenyl)-1,3,4,9-tetrahydro-2H-pyrido[3,4-b]indol-2-yl]- |
| | N,N-dimethyl-6-(trifluoromethyl)-1,3,5-triazin-2-amine; |
| 442 | (1S)-6-chloro-1-(4-methylphenyl)-2-[4-(morpholin-4-yl)-6-(trifluoromethyl)-1,3,5- |
| | triazin-2-yl]-2,3,4,9-tetrahydro-1H-pyrido[3,4-b]indole; |
| 443 | 4-{(1S)-6-chloro-1-[6-(morpholin-4-yl)pyridin-3-yl]-1,3,4,9-tetrahydro-2H-pyrido[3,4- |
| | b]indol-2-yl}-N,N-dimethyl-6-(trifluoromethyl)-1,3,5-triazin-2-amine; |
| 444 | 4-{(1S)-6-chloro-1-[2-(morpholin-4-yl)pyrimidin-5-yl]-1,3,4,9-tetrahydro-2H-pyrido[3,4- |
| | b]indol-2-yl}-N,N-dimethyl-6-(trifluoromethyl)-1,3,5-triazin-2-amine; |
| 445 | (1S)-6-chloro-1-[2-(morpholin-4-yl)pyrimidin-5-yl]-2-[4-(morpholin-4-yl)-6- |
| | (trifluoromethyl)-1,3,5-triazin-2-yl]-2,3,4,9-tetrahydro-1H-pyrido[3,4-b]indole; |
| 446 | (1S)-6-chloro-1-[6-(morpholin-4-yl)pyridin-3-yl]-2-[4-(morpholin-4-yl)-6- |
| | (trifluoromethyl)-1,3,5-triazin-2-yl]-2,3,4,9-tetrahydro-1H-pyrido[3,4-b]indole; |
| 447 | 4-{6-chloro-2-[4-(trifluoromethyl)pyrimidin-2-yl]-2,3,4,9-tetrahydro-1H-pyrido[3,4- |
| | b]indol-1-yl}benzonitrile; |
| 451 | 6-chloro-1-(2,4-difluorophenyl)-2-(4,6-dimethyl-1,3,5-triazin-2-yl)-2,3,4,9-tetrahydro- |
| | 1H-pyrido[3,4-b]indole; |
| 452 | 6-chloro-2-(4,6-dimethyl-1,3,5-triazin-2-yl)-1-(2-fluoro-4-methylphenyl)-2,3,4,9- |
| | tetrahydro-1H-pyrido[3,4-b]indole; |
| 453 | 4-[6-chloro-2-(4,6-dimethyl-1,3,5-triazin-2-yl)-2,3,4,9-tetrahydro-1H-pyrido[3,4-b]indol- |
| | 1-yl]-3-fluorobenzonitrile; |
| 454 | 2-[4,6-bis(trifluoromethyl)pyrimidin-2-yl]-1-[6-(morpholin-4-yl)pyridin-3-yl]-6-(2H- |
| | 1,2,3-triazol-2-yl)-2,3,4,9-tetrahydro-1H-pyrido[3,4-b]indole; |
| 455 | 2-[4,6-bis(trifluoromethyl)pyrimidin-2-yl]-6-(4-methyl-2H-1,2,3-triazol-2-yl)-1-[6- |
| | (morpholin-4-yl)pyridin-3-yl]-2,3,4,9-tetrahydro-1H-pyrido[3,4-b]indole; |
| 457 | (1S)-6-chloro-2-[4-(3,3-difluoroazetidin-1-yl)-6-methyl-1,3,5-triazin-2-yl]-1-[2- |
| | (morpholin-4-yl)pyrimidin-5-yl]-2,3,4,9-tetrahydro-1H-pyrido[3,4-b]indole; |
| 458 | 6-chloro-1-[2-(morpholin-4-yl)pyrimidin-5-yl]-2-[6-(trifluoromethyl)-1,2,4-triazin-3-y]]- |
| | 2,3,4,9-tetrahydro-1H-pyrido[3,4-b]indole; |
| 459 | 6-chloro-2-(4,6-dimethyl-1,3,5-triazin-2-yl)-1-[2-fluoro-6-(morpholin-4-yl)pyridin-3-yl]- |
| | 2,3,4,9-tetrahydro-1H-pyrido[3,4-b]indole; |
| 460 | (1S)-6-chloro-1-[2-(morpholin-4-yl)pyrimidin-5-yl]-2-[4-(trifluoromethyl)-1,3,5-triazin- |
| | 2-yl]-2,3,4,9-tetrahydro-1H-pyrido[3,4-b]indole; |
| 461 | (1S)-6-chloro-1-(2,3-difluoro-4-methylphenyl)-2-[4-(trifluoromethyl)-1,3,5-triazin-2-yl]- |
| | 2,3,4,9-tetrahydro-1H-pyrido[3,4-b]indole; |
| 462 | (1S)-6-chloro-1-[6-(morpholin-4-yl)pyridin-3-yl]-2-[4-(trifluoromethyl)-1,3,5-triazin-2- |
| | yl]-2,3,4,9-tetrahydro-1H-pyrido[3,4-b]indole; |
| 463 | (1S)-2-(5-bromopyrimidin-2-yl)-6-chloro-1-(2,3-difluoro-4-methylphenyl)-2,3,4,9- |
| | tetrahydro-1H-pyrido[3,4-b]indole; |
| 464 | (1S)-6-chloro-1-(2,3-difluoro-4-methylphenyl)-2-[4-methyl-6-(trifluoromethyl)-1,3,5- |
| | triazin-2-yl]-2,3,4,9-tetrahydro-1H-pyrido[3,4-b]indole; |
| 465 | (1S)-6-chloro-2-[4-methyl-6-(trifluoromethyl)-1,3,5-triazin-2-yl]-1-[6-(morpholin-4- |
| | yl)pyridin-3-yl]-2,3,4,9-tetrahydro-1H-pyrido[3,4-b]indole; |
| 466 | (1S)-6-chloro-2-[4-methyl-6-(trifluoromethyl)-1,3,5-triazin-2-yl]-1-[2-(morpholin-4- |
| | yl)pyrimidin-5-yl]-2,3,4,9-tetrahydro-1H-pyrido[3,4-b]indole; |
| 467 | (1S)-6-chloro-1-(4-methylphenyl)-2-[4-methyl-6-(trifluoromethyl)-1,3,5-triazin-2-yl]- |
| | 2,3,4,9-tetrahydro-1H-pyrido[3,4-b]indole; |
| 468 | 6-chloro-1-(4-chloro-2-fluorophenyl)-2-(4,6-dimethyl-1,3,5-triazin-2-yl)-2,3,4,9- |
| | tetrahydro-1H-pyrido[3,4-b]indole; |
| 469 | 6-chloro-1-(2,3-difluoro-4-methylphenyl)-2-(4-phenylpyrimidin-2-yl)-2,3,4,9-tetrahydro- |
| | 1H-pyrido[3,4-b]indole; |
| 470 | (1S)-2-(5-bromopyrimidin-2-yl)-6-chloro-1-[2-(morpholin-4-yl)pyrimidin-5-yl]-2,3,4,9- |
| | tetrahydro-1H-pyrido[3,4-b]indole; |
| 471 | (1S)-6-chloro-1-(2,3-difluoro-4-methylphenyl)-2-(5-phenylpyrimidin-2-yl)-2,3,4,9- |
| | tetrahydro-1H-pyrido[3,4-b]indole; |
| 472 | (1S)-6-chloro-1-[2-(morpholin-4-yl)pyrimidin-5-yl]-2-(5-phenylpyrimidin-2-yl)-2,3,4,9- |
| | tetrahydro-1H-pyrido[3,4-b]indole; |
| 473 | (1S)-6-chloro-1-[6-(morpholin-4-yl)pyridin-3-yl]-2-[4-(trifluoromethyl)pyrimidin-2-yl]- |
| | 2,3,4,9-tetrahydro-1H-pyrido[3,4-b]indole; |
| 474 | (1S)-6-chloro-1-[2-(4-methylpiperazin-1-yl)pyrimidin-5-yl]-2-[4-(methylsulfanyl)-6- |
| | (trifluoromethyl)-1,3,5-triazin-2-yl]-2,3,4,9-tetrahydro-1H-pyrido[3,4-b]indole; |
| 476 | (1S)-6-chloro-1-(2,3-difluoro-4-methylphenyl)-2-[4-fluoro-6-(4-methylpiperazin-1-yl)- |
| | 1,3,5-triazin-2-yl]-2,3,4,9-tetrahydro-1H-pyrido[3,4-b]indole; |
| 477 | (1S)-6-chloro-1-(2,3-difluoro-4-methylphenyl)-2-[4-(4-methylpiperazin-1-yl)-6- |
| | (trifluoromethyl)-1,3,5-triazin-2-yl]-2,3,4,9-tetrahydro-1H-pyrido[3,4-b]indole; |
| 478 | (1S)-6-chloro-2-[4-(4-methylpiperazin-1-yl)-6-(trifluoromethyl)-1,3,5-triazin-2-yl]-1-[2- |
| | (morpholin-4-yl)pyrimidin-5-yl]-2,3,4,9-tetrahydro-1H-pyrido[3,4-b]indole; |
| 480 | (1S)-6-chloro-1-[6-(4-methylpiperazin-1-yl)pyridin-3-yl]-2-[4- |
| | (trifluoromethyl)pyrimidin-2-yl]-2,3,4,9-tetrahydro-1H-pyrido[3,4-b]indole; |
| 481 | (1S)-2-[4,6-bis(trifluoromethyl)pyrimidin-2-yl]-6-chloro-1-[6-(4-methylpiperazin-1- |
| | yl)pyridin-3-yl]-2,3,4,9-tetrahydro-1H-pyrido[3,4-b]indole; |
| 482 | 2-[4,6-bis(trifluoromethyl)pyrimidin-2-yl]-1-[2-(morpholin-4-yl)pyrimidin-5-yl]-6-(2H- |
| | 1,2,3-triazol-2-yl)-2,3,4,9-tetrahydro-1H-pyrido[3,4-b]indole; |
| 483 | 2-[4,6-bis(trifluoromethyl)pyrimidin-2-yl]-6-(4-methyl-2H-1,2,3-triazol-2-yl)-1-[2- |
| | (morpholin-4-yl)pyrimidin-5-yl]-2,3,4,9-tetrahydro-1H-pyrido[3,4-b]indole; |
| 484 | (1S)-6-chloro-1-[2-(4-methylpiperazin-1-yl)pyrimidin-5-yl]-2-[4- |
| | (trifluoromethyl)pyrimidin-2-yl]-2,3,4,9-tetrahydro-1H-pyrido[3,4-b]indole; |
| 485 | (1S)-2-[4,6-bis(trifluoromethyl)pyrimidin-2-yl]-1-[6-(morpholin-4-yl)pyridin-3-yl]-6- |
| | (2H-1,2,3-triazol-2-yl)-2,3,4,9-tetrahydro-1H-pyrido[3,4-b]indole; |
| 486 | (1S)-2-[4,6-bis(trifluoromethyl)-1,3,5-triazin-2-yl]-1-[6-(morpholin-4-yl)pyridin-3-yl]-6- |
| | (2H-1,2,3-triazol-2-yl)-2,3,4,9-tetrahydro-1H-pyrido[3,4-b]indole; |
| 487 | (1S)-2-[4,6-bis(trifluoromethyl)pyrimidin-2-yl]-6-bromo-1-[6-(morpholin-4-yl)pyridin-3- |
| | yl]-2,3,4,9-tetrahydro-1H-pyrido[3,4-b]indole; |
| 488 | (1S)-6-chloro-1-[2-(4-methylpiperazin-1-yl)pyrimidin-5-yl]-2-[4-(trifluoromethyl)-1,3,5- |
| | triazin-2-yl]-2,3,4,9-tetrahydro-1H-pyrido[3,4-b]indole; |
| 489 | (1S)-6-chloro-1-[6-(4-methylpiperazin-1-yl)pyridin-3-yl]-2-[4-(methylsulfanyl)-6- |
| | (trifluoromethyl)-1,3,5-triazin-2-yl]-2,3,4,9-tetrahydro-1H-pyrido[3,4-b]indole; |
| 490 | 2-[4,6-bis(trifluoromethyl)pyrimidin-2-yl]-1-(2,3-difluoro-4-methylphenyl)-6-fluoro- |
| | 2,3,4,9-tetrahydro-1H-pyrido[3,4-b]indole; |
| ¹491 | (1S)-6-chloro-1-[6-(4-methylpiperazin-1-yl)pyridin-3-yl]-2-[4-(trifluoromethyl)-1,3,5- |
| | triazin-2-yl]-2,3,4,9-tetrahydro-1H-pyrido[3,4-b]indole; |
| 492 | 2-[4,6-bis(trifluoromethyl)-1,3,5-triazin-2-yl]-1-(2,3-difluoro-4-methylphenyl)-6-fluoro- |
| | 2,3,4,9-tetrahydro-1H-pyrido[3,4-b]indole; |
| 495 | 1-(2,3-difluoro-4-methylphenyl)-6-fluoro-2-[4-(methylsulfany])-6-(trifluoromethyl)- |
| | 1,3,5-triazin-2-yl]-2,3,4,9-tetrahydro-1H-pyrido[3,4-b]indole; |
| 496 | (1S)-2-[4,6-bis(trifluoromethyl)pyrimidin-2-yl]-6-chloro-1-[2-(3,3-dimethylpiperazin-1- |
| | yl)pyrimidin-5-yl]-2,3,4,9-tetrahydro-1H-pyrido[3,4-b]indole; |
| 497 | 2-[4,6-bis(trifluoromethyl)-1,3,5-triazin-2-yl]-6-chloro-1-(2,3-difluoro-4-methylphenyl)- |
| | 2,3,4,9-tetrahydro-1H-pyrido[3,4-b]indole; |
| 498 | (1S)-1-[6-(morpholin-4-yl)pyridin-3-yl]-6-(2H-1,2,3-triazol-2-yl)-2-[4-(trifluoromethyl)- |
| | 1,3,5-triazin-2-yl]-2,3,4,9-tetrahydro-1H-pyrido[3,4-b]indole; |
| 499 | (1S)-2-[4,6-bis(trifluoromethyl)pyrimidin-2-yl]-6-bromo-1-[2-(4-methylpiperazin-1- |
| | yl)pyrimidin-5-yl]-2,3,4,9-tetrahydro-1H-pyrido[3,4-b]indole; |
| 500 | (1S)-2-[4,6-bis(trifluoromethyl)pyrimidin-2-yl]-1-[2-(4-methylpiperazin-1-yl)pyrimidin- |
| | 5-yl]-6-(2H-1,2,3-triazol-2-yl)-2,3,4,9-tetrahydro-1H-pyrido[3,4-b]indole; |
| 501 | (1S)-2-[4,6-bis(trifluoromethyl)-1,3,5-triazin-2-yl]-1-[2-(4-methylpiperazin-1- |
| | yl)pyrimidin-5-yl]-6-(2H-1,2,3-triazol-2-yl)-2,3,4,9-tetrahydro-1H-pyrido[3,4-b]indole; |
| 502 | 1-(2,3-difluoro-4-methylphenyl)-6-(2H-1,2,3-triazol-2-yl)-2-[4- |
| | (trifluoromethyl)pyrimidin-2-yl]-2,3,4,9-tetrahydro-1H-pyrido[3,4-b]indole; |
| 503 | (1S)-2-[4,6-bis(trifluoromethyl)pyrimidin-2-yl]-6-chloro-1-{2-[(3R,5S)-3,5- |
| | dimethylpiperazin-1-yl]pyrimidin-5-yl}-2,3,4,9-tetrahydro-1H-pyrido[3,4-b]indole; |
| 505 | (1S)-6-chloro-1-(2,3-difluoro-4-methylphenyl)-2-[4-(trichloromethyl)-6- |
| | (trifluoromethyl)-1,3,5-triazin-2-yl]-2,3,4,9-tetrahydro-1H-pyrido[3,4-b]indole; |
| 506 | (1S)-1-[2-(4-methylpiperazin-1-yl)pyrimidin-5-yl]-6-(2H-1,2,3-triazol-2-yl)-2-[4- |
| | (trifluoromethyl)-1,3,5-triazin-2-yl]-2,3,4,9-tetrahydro-1H-pyrido[3,4-b]indole; |
| 507 | (1S)-6-chloro-2-[4-(difluoromethyl)-6-(trifluoromethyl)-1,3,5-triazin-2-yl]-1-[2- |
| | (morpholin-4-yl)pyrimidin-5-yl]-2,3,4,9-tetrahydro-1H-pyrido[3,4-b]indole; |
| 508 | (1S)-6-chloro-2-[4-(difluoromethyl)-6-(trifluoromethyl)-1,3,5-triazin-2-yl]-1-[2-(4- |
| | methylpiperazin-1-yl)pyrimidin-5-yl]-2,3,4,9-tetrahydro-1H-pyrido[3,4-b]indole; |
| 509 | (1S)-6-chloro-1-[2-(4-methylpiperazin-1-yl)pyrimidin-5-yl]-2-[4-(morpholin-4-yl)-6- |
| | (trifluoromethyl)-1,3,5-triazin-2-yl]-2,3,4,9-tetrahydro-1H-pyrido[3,4-b]indole; |
| 510 | (1S)-6-chloro-1-(2,3-difluoro-4-methylphenyl)-2-[4-(difluoromethyl)-6-(trifluoromethyl)- |
| | 1,3,5-triazin-2-yl]-2,3,4,9-tetrahydro-1H-pyrido[3,4-b]indole; |
| 511 | (1S)-2-[4,6-bis(trifluoromethyl)pyrimidin-2-yl]-6-chloro-1-{2-[(3R)-3-methylpiperazin-1- |
| | yl]pyrimidin-5-yl}-2,3,4,9-tetrahydro-1H-pyrido[3,4-b]indole; |
| 512 | (1S)-2-[4,6-bis(trifluoromethyl)pyrimidin-2-yl]-6-chloro-1-[2-(4,7-diazaspiro[2.5]octan- |
| | 7-yl)pyrimidin-5-yl]-2,3,4,9-tetrahydro-1H-pyrido[3,4-b]indole; |
| 514 | (1S)-2-[4,6-bis(trifluoromethyl)pyrimidin-2-yl]-1-[2-(morpholin-4-yl)pyrimidin-5-yl]-6- |
| | (2H-1,2,3-triazol-2-yl)-2,3,4,9-tetrahydro-1H-pyrido[3,4-b]indole; |
| 515 | (1S)-1-[2-(morpholin-4-yl)pyrimidin-5-yl]-6-(2H-1,2,3-triazol-2-yl)-2-[4- |
| | (trifluoromethyl)-1,3,5-triazin-2-yl]-2,3,4,9-tetrahydro-1H-pyrido[3,4-b]indole; |
| 516 | (1S)-2-[4,6-bis(trifluoromethyl)-1,3,5-triazin-2-yl]-1-[2-(morpholin-4-yl)pyrimidin-5-yl]- |
| | 6-(2H-1,2,3-triazol-2-yl)-2,3,4,9-tetrahydro-1H-pyrido[3,4-b]indole; |
| 517 | (1S)-2-[4,6-bis(trifluoromethyl)pyrimidin-2-yl]-6-chloro-1-{2-[(3S)-3-methylpiperazin-1- |
| | yl]pyrimidin-5-yl}-2,3,4,9-tetrahydro-1H-pyrido[3,4-b]indole; |
| 518 | (1S)-2-[4,6-bis(trifluoromethyl)pyrimidin-2-yl]-6-bromo-1-(2,3-difluoro-4- |
| | methylphenyl)-2,3,4,9-tetrahydro-1H-pyrido[3,4-b]indole; |
| 519 | (1S)-2-[4,6-bis(trifluoromethyl)pyrimidin-2-yl]-1-(2,3-difluoro-4-methylphenyl)-6-(2H- |
| | 1,2,3-triazol-2-yl)-2,3,4,9-tetrahydro-1H-pyrido[3,4-b]indole; |
| 520 | (1S)-2-[4,6-bis(trifluoromethyl)pyrimidin-2-yl]-1-(2,3-difluoro-4-methylphenyl)-6-(4- |
| | methyl-2H-1,2,3-triazol-2-yl)-2,3,4,9-tetrahydro-1H-pyrido[3,4-b]indole; |
| 521 | {4-[(1S)-6-chloro-1-(2,3-difluoro-4-methylphenyl)-1,3,4,9-tetrahydro-2H-pyrido[3,4- |
| | b]indol-2-yl]-6-(trifluoromethyl)-1,3,5-triazin-2-yl}methanol; |
| 522 | (1S)-2-[4,6-bis(trifluoromethyl)-1,3,5-triazin-2-yl]-6-chloro-1-(2-methoxypyrimidin-5- |
| | yl)-2,3,4,9-tetrahydro-1H-pyrido[3,4-b]indole; |
| 523 | (1S)-2-[4,6-bis(trifluoromethyl)-1,3,5-triazin-2-yl]-6-bromo-1-(2,3-difluoro-4- |
| | methylphenyl)-2,3,4,9-tetrahydro-1H-pyrido[3,4-b]indole; |
| 524 | (1S)-2-[4,6-bis(trifluoromethyl)-1,3,5-triazin-2-yl]-1-(2,3-difluoro-4-methylphenyl)-6- |
| | (2H-1,2,3-triazol-2-yl)-2,3,4,9-tetrahydro-1H-pyrido[3,4-b]indole; |
| 525 | (1S)-2-[4,6-bis(trifluoromethyl)-1,3,5-triazin-2-yl]-1-(2,3-difluoro-4-methylphenyl)-6-(4- |
| | methyl-2H-1,2,3-triazol-2-yl)-2,3,4,9-tetrahydro-1H-pyrido[3,4-b]indole; |
| 526 | (1S)-6-chloro-1-(2,3-difluoro-4-methylphenyl)-2-[4-(fluoromethyl)-6-(trifluoromethyl)- |
| | 1,3,5-triazin-2-yl]-2,3,4,9-tetrahydro-1H-pyrido[3,4-b]indole; |
| 527 | (1S)-2-[4,6-bis(trifluoromethyl)-1,3,5-triazin-2-yl]-1-(2,3-difluoro-4-methylphenyl)-6- |
| | nitro-2,3,4,9-tetrahydro-1H-pyrido[3,4-b]indole; |
| 528 | (1S)-2-[4,6-bis(trifluoromethyl)pyrimidin-2-yl]-1-(2,3-difluoro-4-methylphenyl)-6- |
| | methoxy-2,3,4,9-tetrahydro-1H-pyrido[3,4-b]indole; |
| 529 | (1S)-2-[4,6-bis(trifluoromethyl)-1,3,5-triazin-2-yl]-1-(2,3-difluoro-4-methylphenyl)-6-(4- |
| | methyl-1H-pyrazol-1-yl)-2,3,4,9-tetrahydro-1H-pyrido[3,4-b]indole; |
| 531 | N²-(5-{(1S)-2-[4,6-bis(trifluoromethyl)-1,3,5-triazin-2-yl]-6-chloro-2,3,4,9-tetrahydro- |
| | 1H-pyrido[3,4-b]indol-1-yl}pyrimidin-2-yl)-N¹,N¹-dimethylethane-1,2-diamine; |
| 532 | methyl 5-{(1S)-2-[4,6-bis(trifluoromethyl)-1,3,5-triazin-2-yl]-6-chloro-2,3,4,9-tetrahydro- |
| | 1H-pyrido[3,4-b]indol-1-yl}pyrimidine-2-carboxylate; |
| 533 | 1-(5-{(1S)-2-[4,6-bis(trifluoromethyl)-1,3,5-triazin-2-yl]-6-chloro-2,3,4,9-tetrahydro-1H- |
| | pyrido[3,4-b]indol-1-yl}pyrimidin-2-yl)ethan-1-one; |
| 534 | (1S)-2-[4,6-bis(trifluoromethyl)-1,3,5-triazin-2-yl]-6-chloro-1-[2-(2- |
| | methoxyethoxy)pyrimidin-5-yl]-2,3,4,9-tetrahydro-1H-pyrido[3,4-b]indole; |
| 535 | 2-[(5-{(1S)-2-[4,6-bis(trifluoromethyl)-1,3,5-triazin-2-yl]-6-chloro-2,3,4,9-tetrahydro- |
| | 1H-pyrido[3,4-b]indol-1-yl}pyrimidin-2-yl)amino]ethan-1-ol; |
| 536 | 2-[4,6-bis(trifluoromethyl)-1,3,5-triazin-2-yl]-6-chloro-1-[2-(2- |
| | methoxyethoxy)pyrimidin-5-yl]-2,3,4,9-tetrahydro-1H-pyrido[3,4-b]indole; |
| 537 | N-(5-{(1S)-2-[4,6-bis(trifluoromethyl)-1,3,5-triazin-2-yl]-6-chloro-2,3,4,9-tetrahydro-1H- |
| | pyrido[3,4-b]indol-1-yl}pyrimidin-2-yl)-β-alanine; and, |
| 538 | 4-[(5-{(1S)-2-[4,6-bis(trifluoromethyl)-1,3,5-triazin-2-yl]-6-chloro-2,3,4,9-tetrahydro- |
| | 1H-pyrido[3,4-b]indol-1-yl}pyrimidin-2-yl)amino]butanoic acid; |

wherein the form of the compound is selected from the group consisting of a salt, hydrate, solvate, racemate, enantiomer, diastereomer, stereoisomer, and tautomer form thereof.

Another aspect of the compound of Formula (I) or a form thereof includes a compound (Cpd) salt having dihydroorotate dehydrogenase inhibition activity ≤10.0 nM selected from the group consisting of:

| Cpd | Name |
|---|---|
| 357 | 2-[4,6-bis(trifluoromethyl)pyrimidin-2-yl]-6-chloro-1-[6-(morpholin-4-yl)pyridin-3-yl]- |
| | 2,3,4,9-tetrahydro-1H-pyrido[3,4-b]indole formate (1:1); |
| 358 | 2-[4,6-bis(trifluoromethyl)pyrimidin-2-yl]-6-chloro-1-[2-(morpholin-4-yl)pyrimidin-5- |
| | yl]-2,3,4,9-tetrahydro-1H-pyrido[3,4-b]indole formate (1:1); and, |
| 491 | (1S)-6-chloro-1-[6-(4-methylpiperazin-1-yl)pyridin-3-yl]-2-[4-(trifluoromethyl)-1,3,5- |
| | triazin-2-yl]-2,3,4,9-tetrahydro-1H-py.rido[3,4-b]indole formate (1:1); |

wherein the form of the compound is selected from the group consisting of a hydrate, solvate, racemate, enantiomer, diastereomer, stereoisomer, and tautomer form thereof.

An aspect of the compound of Formula (I) or a form thereof includes a compound (Cpd) having dihydroorotate dehydrogenase inhibition activity ≤1.0 nM selected from the group consisting of (wherein ¹Cpd #indicates the compound was isolated as a salt form):

| Cpd | Name |
|---|---|
| 7 | 6-chloro-2-(4,6-dichloropyrimidin-2-yl)-1-(4-methylphenyl)-2,3,4,9-tetrahydro-1H- |
| | pyrido[3,4-b]indole; |
| 118 | 6-chloro-1-(2,3-difluoro-4-methylphenyl)-5-fluoro-2-(5-fluoro-4,6-dimethylpyrimidin-2- |
| | yl)-2,3,4,9-tetrahydro-1H-pyrido[3,4-b]indole; |
| 128 | 6-chloro-1-(2,3-difluoro-4-methylphenyl)-2-[4-(trifluoromethyl)pyrimidin-2-yl]-2,3,4,9- |
| | tetrahydro-1H-pyrido[3,4-b]indole; |
| 140 | 6-chloro-1-(2,3-difluoro-4-methylphenyl)-7-fluoro-2-[4-(trifluoromethyl)pyrimidin-2-yl]- |
| | 2,3,4,9-tetrahydro-1H-pyrido[3,4-b]indole; |
| 144 | 6-chloro-1-(4-chloro-2,3-difluorophenyl)-2-[4-(trifluoromethyl)pyrimidin-2-yl]-2,3,4,9- |
| | tetrahydro-1H-pyrido[3,4-b]indole; |
| 148 | 6-chloro-1-(2,3-difluoro-4-methylphenyl)-7-fluoro-2-[4-methyl-6- |
| | (trifluoromethyl)pyrimidin-2-yl]-2,3,4,9-tetrahydro-1H-pyrido[3,4-b]indole; |
| 154 | 2-[4,6-bis(trifluoromethyl)pyrimidin-2-yl]-6-chloro-7-fluoro-1-(4-methylphenyl)-2,3,4,9- |
| | tetrahydro-1H-pyrido[3,4-b]indole; |
| 155 | 2-[4,6-bis(trifluoromethyl)pyrimidin-2-yl]-6-chloro-1-(2,3-difluoro-4-methylphenyl)-7- |
| | fluoro-2,3,4,9-tetrahydro-1H-pyrido[3,4-b]indole; |
| 157 | 2-[4,6-bis(trifluoromethyl)pyrimidin-2-yl]-6-chloro-1-(2,3-difluorophenyl)-7-fluoro- |
| | 2,3,4,9-tetrahydro-1H-pyrido[3,4-b]indole; |
| 188 | 4-{2-[4,6-bis(trifluoromethyl)pyrimidin-2-yl]-6-chloro-7-fluoro-2,3,4,9-tetrahydro-1H- |
| | pyrido[3,4-b]indol-1-yl}benzonitrile; |
| 189 | (1S)-6-chloro-1-(2,3-difluoro-4-methylphenyl)-5-fluoro-2-(5-fluoro-4,6- |
| | dimethylpyrimidin-2-yl)-2,3,4,9-tetrahydro-1H-pyrido[3,4-b]indole; |
| 195 | 6-chloro-2-(5-chloropyrimidin-2-yl)-1-(2,3-difluoro-4-methylphenyl)-5-fluoro-2,3,4,9- |
| | tetrahydro-1H-pyrido[3,4-b]indole; |
| 205 | 6-chloro-1-(2,3-difluoro-4-methylphenyl)-2-(4,6-dimethylpyrimidin-2-yl)-5-fluoro- |
| | 2,3,4,9-tetrahydro-1H-pyrido[3,4-b]indole; |
| 206 | 6-chloro-1-(2,3-difluoro-4-methylphenyl)-5-fluoro-2-[4-methyl-6- |
| | (trifluoromethyl)pyrimidin-2-yl]-2,3,4,9-tetrahydro-1H-pyrido[3,4-b]indole; |
| 209 | 2-[4,6-bis(trifluoromethyl)pyrimidin-2-yl]-6-chloro-1-(2,3-difluoro-4-methylphenyl)-5- |
| | fluoro-2,3,4,9-tetrahydro-1H-pyrido[3,4-b]indole; |
| 214 | 2-[6-chloro-1-(2,3-difluoro-4-methylphenyl)-5-fluoro-1,3,4,9-tetrahydro-2H-pyrido[3,4- |
| | b]indol-2-yl]-5-fluoropyrimidine-4-carbonitrile; |
| 232 | 2-[6-chloro-1-(2,3-difluoro-4-methylphenyl)-5-fluoro-1,3,4,9-tetrahydro-2H-pyrido[3,4- |
| | b]indol-2-yl]-6-methylpyrimidine-4-carbonitrile; |
| 233 | (1S)-6-chloro-1-(2,3-difluoro-4-methylphenyl)-2-[4-(trifluoromethyl)pyrimidin-2-yl]- |
| | 2,3,4,9-tetrahydro-1H-pyrido[3,4-b]indole; |
| 234 | (1S)-6-chloro-1-(2,3-difluoro-4-methylphenyl)-2-(4-methylpyrimidin-2-yl)-2,3,4,9- |
| | tetrahydro-1H-pyrido[3,4-b]indole; |
| 238 | (1S)-6-chloro-1-(2,3-difluoro-4-methylphenyl)-2-(5-fluoropyrimidin-2-yl)-2,3,4,9- |
| | tetrahydro-1H-pyrido[3,4-b]indole; |
| 248 | 6-chloro-1-(2,3-difluoro-4-methylphenyl)-2-[4-(trifluoromethyl)pyrimidin-2-yl]-2,3,4,9- |
| | tetrahydro-1H-pyrido[3,4-b]indol-8-amine; |
| 259 | 6-chloro-1-(2-fluoro-3-methylphenyl)-2-[4-(trifluoromethyl)pyrimidin-2-yl]-2,3,4,9- |
| | tetrahydro-1H-pyrido[3,4-b]indole; |
| 268 | 1-(4-bromo-2-fluorophenyl)-6-chloro-2-[4-(trifluoromethyl)pyrimidin-2-yl]-2,3,4,9- |
| | tetrahydro-1H-pyrido[3,4-b]indole; |
| 270 | 1-(4-bromo-2,3-difluorophenyl)-6-chloro-2-[4-(trifluoromethyl)pyrimidin-2-yl]-2,3,4,9- |
| | tetrahydro-1H-pyrido[3,4-b]indole; |
| 276 | 1-(2,3-difluoro-4-methylphenyl)-6-methyl-2-[4-(trifluoromethyl)pyrimidin-2-yl]-2,3,4,9- |
| | tetrahydro-1H-pyrido[3,4-b]indol-8-ol; |
| 283 | 1-(4-bromo-2,3-difluorophenyl)-6-chloro-2-(4,6-dimethyl-1,3,5-triazin-2-yl)-2,3,4,9- |
| | tetrahydro-1H-pyrido[3,4-b]indole; |
| 305 | 1-(2,3-difluoro-4-methylphenyl)-6-nitro-2-[4-(trifluoromethyl)pyrimidin-2-yl]-2,3,4,9- |
| | tetrahydro-1H-pyrido[3,4-b]indole; |
| 340 | 2-[4,6-bis(trifluoromethyl)pyrimidin-2-yl]-6-chloro-1-(2,3-difluoro-4-methylphenyl)- |
| | 2,3,4,9-tetrahydro-1H-pyrido[3,4-b]indole; |
| 342 | 6-chloro-1-(2,3-difluoro-4-methylphenyl)-2-(4,6-dimethylpyrimidin-2-yl)-2,3,4,9- |
| | tetrahydro-1H-pyrido[3,4-b]indole; |
| ¹357 | 2-[4,6-bis(trifluoromethyl)pyrimidin-2-yl]-6-chloro-1-[6-(morpholin-4-yl)pyridin-3-yl]- |
| | 2,3,4,9-tetrahydro-1H-pyrido[3,4-b]indole; |
| ¹358 | 2-[4,6-bis(trifluoromethyl)pyrimidin-2-yl]-6-chloro-1-[2-(morpholin-4-yl)pyrimidin-5- |
| | yl]-2,3,4,9-tetrahydro-1H-pyrido[3,4-b]indole; |
| 360 | 2-[4,6-bis(trifluoromethyl)pyrimidin-2-yl]-6-chloro-1-(4-methylphenyl)-2,3,4,9- |
| | tetrahydro-1H-pyrido[3,4-b]indole; |
| 381 | 2-[4,6-bis(trifluoromethyl)pyrimidin-2-yl]-6-chloro-1-[2-(4-methylpiperazin-1- |
| | yl)pyrimidin-5-yl]-2,3,4,9-tetrahydro-1H-pyrido[3,4-b]indole; |
| 386 | (1S)-2-[4,6-bis(trifluoromethyl)-1,3,5-triazin-2-yl]-6-chloro-1-(4-methylphenyl)-2,3,4,9- |
| | tetrahydro-1H-pyrido[3,4-b]indole; |
| 387 | (1S)-2-[4,6-bis(trifluoromethyl)-1,3,5-triazin-2-yl]-6-chloro-1-(2,3-difluoro-4- |
| | methylphenyl)-2,3,4,9-tetrahydro-1H-pyrido[3,4-b]indole; |
| 388 | 2-[4,6-bis(trifluoromethyl)-1,3,5-triazin-2-yl]-6-chloro-1-[2-(morpholin-4-yl)pyrimidin-5- |
| | yl]-2,3,4,9-tetrahydro-1H-pyrido[3,4-b]indole; |
| 389 | (1S)-6-chloro-1-(2,3-difluoro-4-methylphenyl)-2-[4-(methylsulfanyl)-6-(trifluoromethyl)- |
| | 1,3,5-triazin-2-yl]-2,3,4,9-tetrahydro-1H-pyrido[3,4-b]indole; |
| 403 | 6-chloro-1-(2,3-difluoro-4-methylphenyl)-2-[5-(trifluoromethyl)-1,2,4-triazin-3-yl]- |
| | 2,3,4,9-tetrahydro-1H-pyrido[3,4-b]indole; |
| 404 | 2-[4,6-bis(trifluoromethyl)-1,3,5-triazin-2-yl]-6-chloro-1-[6-(morpholin-4-y])pyridin-3- |
| | yl]-2,3,4,9-tetrahydro-1H-pyrido[3,4-b]indole; |
| 405 | (1S)-2-[4,6-bis(trifluoromethyl)-1,3,5-triazin-2-yl]-6-chloro-1-[6-(morpholin-4- |
| | yl)pyridin-3-yl]-2,3,4,9-tetrahydro-1H-pyrido[3,4-b]indole; |
| 406 | (1S)-2-[4,6-bis(trifluoromethyl)-1,3,5-triazin-2-yl]-6-chloro-1-[2-(morpholin-4- |
| | yl)pyrimidin-5-yl]-2,3,4,9-tetrahydro-1H-pyrido[3,4-b]indole; |
| 407 | (1S)-2-[4,6-bis(trifluoromethyl)pyrimidin-2-yl]-6-chloro-1-(2,3-difluoro-4- |
| | methylphenyl)-2,3,4,9-tetrahydro-1H-pyrido[3,4-b]indole; |
| 409 | (1S)-2-[4,6-bis(trifluoromethyl)pyrimidin-2-yl]-6-chloro-1-[6-(morpholin-4-yl)pyridin-3- |
| | yl]-2,3,4,9-tetrahydro-1H-pyrido[3,4-b]indole; |
| 410 | (1S)-2-[4,6-bis(trifluoromethyl)pyrimidin-2-yl]-6-chloro-1-[2-(morpholin-4-yl)pyrimidin- |
| | 5-yl]-2,3,4,9-tetrahydro-1H-pyrido[3,4-b]indole; |
| 412 | (1S)-6-chloro-1-(2,3-difluoro-4-methylphenyl)-2-(5-fluoro-4,6-dimethylpyrimidin-2-yl)- |
| | 2,3,4,9-tetrahydro-1H-pyrido[3,4-b]indole; |
| 413 | 2-[(1S)-6-chloro-1-(2,3-difluoro-4-methylphenyl)-1,3,4,9-tetrahydro-2H-pyrido[3,4- |
| | b]indol-2-yl]-5-fluoro-6-methylpyrimidine-4-carbonitrile; |
| 419 | (1S)-2-[4,6-bis(trifluoromethyl)pyrimidin-2-yl]-6-chloro-1-[2-(4-methylpiperazin-1- |
| | yl)pyrimidin-5-yl]-2,3,4,9-tetrahydro-1H-pyrido[3,4-b]indole; |
| 420 | (1S)-2-[4,6-bis(trifluoromethyl)-1,3,5-triazin-2-yl]-6-chloro-1-[2-(4-methylpiperazin-1- |
| | yl)pyrimidin-5-yl]-2,3,4,9-tetrahydro-1H-pyrido[3,4-b]indole; |
| 436 | (1S)-6-chloro-1-(2,3-difluoro-4-methylphenyl)-2-[4-(morpholin-4-yl)-6-(trifluoromethyl)- |
| | 1,3,5-triazin-2-yl]-2,3,4,9-tetrahydro-1H-pyrido[3,4-b]indole; |
| 438 | 6-chloro-1-(2,3-difluoro-4-methylphenyl)-2-[4-methyl-6-(trifluoromethyl)-1,3,5-triazin-2- |
| | yl]-2,3,4,9-tetrahydro-1H-pyrido[3,4-b]indole; |
| 454 | 2-[4,6-bis(trifluoromethyl)pyrimidin-2-yl]-1-[6-(morpholin-4-yl)pyridin-3-yl]-6-(2H- |
| | 1,2,3-triazol-2-yl)-2,3,4,9-tetrahydro-1H-pyrido[3,4-b]indole; |
| 461 | (1S)-6-chloro-1-(2,3-difluoro-4-methylphenyl)-2-[4-(trifluoromethyl)-1,3,5-triazin-2-yl]- |
| | 2,3,4,9-tetrahydro-1H-pyrido[3,4-b]indole; |
| 464 | (1S)-6-chloro-1-(2,3-difluoro-4-methylphenyl)-2-[4-methyl-6-(trifluoromethyl)-1,3,5- |
| | triazin-2-yl]-2,3,4,9-tetrahydro-1H-pyrido[3,4-b]indole; |
| 465 | (1S)-6-chloro-2-[4-methyl-6-(trifluoromethyl)-1,3,5-triazin-2-yl]-1-[6-(morpholin-4- |
| | yl)pyridin-3-yl]-2,3,4,9-tetrahydro-1H-pyrido[3,4-b]indole; |
| 466 | (1S)-6-chloro-2-[4-methyl-6-(trifluoromethyl)-1,3,5-triazin-2-yl]-1-[2-(morpholin-4- |
| | yl)pyrimidin-5-yl]-2,3,4,9-tetrahydro-1H-pyrido[3,4-b]indole; |
| 467 | (1S)-6-chloro-1-(4-methylphenyl)-2-[4-methyl-6-(trifluoromethyl)-1,3,5-triazin-2-yl]- |
| | 2,3,4,9-tetrahydro-1H-pyrido[3,4-b]indole; |
| 473 | (1S)-6-chloro-1-[6-(morpholin-4-yl)pyridin-3-yl]-2-[4-(trifluoromethyl)pyrimidin-2-yl]- |
| | 2,3,4,9-tetrahydro-1H-pyrido[3,4-b]indole; |
| 481 | (1S)-2-[4,6-bis(trifluoromethyl)pyrimidin-2-yl]-6-chloro-1-[6-(4-methylpiperazin-1- |
| | yl)pyridin-3-yl]-2,3,4,9-tetrahydro-1H-pyrido[3,4-b]indole; |
| 482 | 2-[4,6-bis(trifluoromethyl)pyrimidin-2-yl]-1-[2-(morpholin-4-yl)pyrimidin-5-yl]-6-(2H- |
| | 1,2,3-triazol-2-yl)-2,3,4,9-tetrahydro-1H-pyrido[3,4-b]indole; |
| 485 | (1S)-2-[4,6-bis(trifluoromethyl)pyrimidin-2-yl]-1-[6-(morpholin-4-yl)pyridin-3-yl]-6- |
| | (2H-1,2,3-triazol-2-yl)-2,3,4,9-tetrahydro-1H-pyrido[3,4-b]indole; |
| 486 | (1S)-2-[4,6-bis(trifluoromethyl)-1,3,5-triazin-2-yl]-1-[6-(morpholin-4-yl)pyridin-3-yl]-6- |
| | (2H-1,2,3-triazol-2-yl)-2,3,4,9-tetrahydro-1H-pyrido[3,4-b]indole; |
| 487 | (1S)-2-[4,6-bis(trifluoromethyl)pyrimidin-2-yl]-6-bromo-1-[6-(morpholin-4-yl)pyridin-3- |
| | yl]-2,3,4,9-tetrahydro-1H-pyrido[3,4-b]indole; |
| 497 | 2-[4,6-bis(trifluoromethyl)-1,3,5-triazin-2-yl]-6-chloro-1-(2,3-difluoro-4-methylphenyl)- |
| | 2,3,4,9-tetrahydro-1H-pyrido[3,4-b]indole; |
| 498 | (1S)-1-[6-(morpholin-4-yl)pyridin-3-yl]-6-(2H-1,2,3-triazol-2-yl)-2-[4-(trifluoromethyl)- |
| | 1,3,5-triazin-2-yl]-2,3,4,9-tetrahydro-1H-pyrido[3,4-b]indole; |
| 499 | (1S)-2-[4,6-bis(trifluoromethyl)pyrimidin-2-yl]-6-bromo-1-[2-(4-methylpiperazin-1- |
| | yl)pyrimidin-5-yl]-2,3,4,9-tetrahydro-1H-pyrido[3,4-b]indole; |
| 500 | (1S)-2-[4,6-bis(trifluoromethyl)pyrimidin-2-yl]-1-[2-(4-methylpiperazin-1-yl)pyrimidin- |
| | 5-yl]-6-(2H-1,2,3-triazol-2-yl)-2,3,4,9-tetrahydro-1H-pyrido[3,4-b]indole; |
| 507 | (1S)-6-chloro-2-[4-(difluoromethyl)-6-(trifluoromethyl)-1,3,5-triazin-2-yl]-1-[2- |
| | (morpholin-4-yl)pyrimidin-5-yl]-2,3,4,9-tetrahydro-1H-pyrido[3,4-b]indole; |
| 508 | (1S)-6-chloro-2-[4-(difluoromethyl)-6-(trifluoromethyl)-1,3,5-triazin-2-yl]-1-[2-(4- |
| | methylpiperazin-1-yl)pyrimidin-5-yl]-2,3,4,9-tetrahydro-1H-pyrido[3,4-b]indole; |
| 510 | (1S)-6-chloro-1-(2,3-difluoro-4-methylphenyl)-2-[4-(difluoromethyl)-6-(trifluoromethyl)- |
| | 1,3,5-triazin-2-yl]-2,3,4,9-tetrahydro-1H-pyrido[3,4-b]indole; |
| 514 | (1S)-2-[4,6-bis(trifluoromethyl)pyrimidin-2-yl]-1-[2-(morpholin-4-yl)pyrimidin-5-yl]-6- |
| | (2H-1,2,3-triazol-2-yl)-2,3,4,9-tetrahydro-1H-pyrido[3,4-b]indole; |
| 518 | (1S)-2-[4,6-bis(trifluoromethyl)pyrimidin-2-yl]-6-bromo-1-(2,3-difluoro-4- |
| | methylphenyl)-2,3,4,9-tetrahydro-1H-pyrido[3,4-b]indole; |
| 519 | (1S)-2-[4,6-bis(trifluoromethyl)pyrimidin-2-yl]-1-(2,3-difluoro-4-methylphenyl)-6-(2H- |
| | 1,2,3-triazol-2-yl)-2,3,4,9-tetrahydro-1H-pyrido[3,4-b]indole; |
| 522 | (1S)-2-[4,6-bis(trifluoromethyl)-1,3,5-triazin-2-yl]-6-chloro-1-(2-methoxypyrimidin-5- |
| | yl)-2,3,4,9-tetrahydro-1H-pyrido[3,4-b]indole; |
| 523 | (1S)-2-[4,6-bis(trifluoromethyl)-1,3,5-triazin-2-yl]-6-bromo-1-(2,3-difluoro-4- |
| | methylphenyl)-2,3,4,9-tetrahydro-1H-pyrido[3,4-b]indole; |
| 524 | (1S)-2-[4,6-bis(trifluoromethyl)-1,3,5-triazin-2-yl]-1-(2,3-difluoro-4-methylphenyl)-6- |
| | (2H-1,2,3-triazol-2-yl)-2,3,4,9-tetrahydro-1H-pyrido[3,4-b]indole; |
| 526 | (1S)-6-chloro-1-(2,3-difluoro-4-methylphenyl)-2-[4-(fluoromethyl)-6-(trifluoromethyl)- |
| | 1,3,5-triazin-2-yl]-2,3,4,9-tetrahydro-1H-pyrido[3,4-b]indole; |
| 527 | (1S)-2-[4,6-bis(trifluoromethyl)-1,3,5-triazin-2-yl]-1-(2,3-difluoro-4-methylphenyl)-6- |
| | nitro-2,3,4,9-tetrahydro-1H-pyrido[3,4-b]indole; |
| 534 | (1S)-2-[4,6-bis(trifluoromethyl)-1,3,5-triazin-2-yl]-6-chloro-1-[2-(2- |
| | methoxyethoxy)pyrimidin-5-yl]-2,3,4,9-tetrahydro-1H-pyrido[3,4-b]indole; |
| 535 | 2-[(5-{(1S)-2-[4,6-bis(trifluoromethyl)-1,3,5-triazin-2-yl]-6-chloro-2,3,4,9-tetrahydro-1H- |
| | pyrido[3,4-b]indol-1-yl}pyrimidin-2-yl)amino]ethan-1-ol; and, |
| 538 | 4-[(5-{(1S)-2-[4,6-bis(trifluoromethyl)-1,3,5-triazin-2-yl]-6-chloro-2,3,4,9-tetrahydro-1H- |
| | pyrido[3,4-b]indol-1-yl}pyrimidin-2-yl)amino]butanoic acid; |

wherein the form of the compound is selected from the group consisting of a salt, hydrate, solvate, racemate, enantiomer, diastereomer, stereoisomer, and tautomer form thereof.

Another aspect of the compound of Formula (I) or a form thereof includes a compound (Cpd) salt having dihydroorotate dehydrogenase inhibition activity ≤10.0 nM selected from the group consisting of:

| Cpd | Name |
|---|---|
| 357 | 2-[4,6-bis(trifluoromethyl)pyrimidin-2-yl]-6-chloro-1-[6-(morpholin-4-yl)pyridin-3-yl]- |
| | 2,3,4,9-tetrahydro-1H-pyrido[3,4-b]indole formate (1:1); and, |
| 358 | 2-[4,6-bis(trifluoromethyl)pyrimidin-2-yl]-6-chloro-1-[2-(morpholin-4-yl)pyrimidin-5- |
| | yl]-2,3,4,9-tetrahydro-1H-pyrido[3,4-b]indole formate (1:1); |

wherein the form of the compound is selected from the group consisting of a hydrate, solvate, racemate, enantiomer, diastereomer, stereoisomer, and tautomer form thereof.

One aspect of the compound of Formula (I) or a form thereof includes a compound (Cpd) selected from the group consisting of (wherein ¹Cpd #indicates the compound was isolated as a salt form):

| Cpd | Name |
|---|---|
| 1 | 6-chloro-2-(5-chloropyrimidin-2-yl)-1-(4-fluorophenyl)-2,3,4,9-tetrahydro-1H- |
| | pyrido[3,4-b]indole; |
| 2 | 6-chloro-1-(4-fluorophenyl)-2-(5-methylpyrimidin-2-yl)-2,3,4,9-tetrahydro-1H- |
| | pyrido[3,4-b]indole; |
| 3 | 6-chloro-1-(4-fluorophenyl)-2-(5-fluoropyrimidin-2-yl)-2,3,4,9-tetrahydro-1H- |
| | pyrido[3,4-b]indole; |
| 4 | 6-chloro-2-(5-chloropyrimidin-2-yl)-7-fluoro-1-(4-fluorophenyl)-2,3,4,9-tetrahydro- |
| | 1H-pyrido[3,4-b]indole; |
| 5 | 6-chloro-7-fluoro-1-(4-fluorophenyl)-2-(5-fluoropyrimidin-2-yl)-2,3,4,9-tetrahydro- |
| | 1H-pyrido[3,4-b]indole; |
| 6 | 6-chloro-7-fluoro-2-(5-fluoropyrimidin-2-yl)-1-(4-methylphenyl)-2,3,4,9-tetrahydro- |
| | 1H-pyrido[3,4-b]indole; |
| 7 | 6-chloro-2-(4,6-dichloropyrimidin-2-yl)-1-(4-methylphenyl)-2,3,4,9-tetrahydro-1H- |
| | pyrido[3,4-b]indole; |
| 8 | 6-chloro-7-fluoro-2-(5-fluoropyrimidin-2-yl)-1-(6-methoxypyridin-3-yl)-2,3,4,9- |
| | tetrahydro-1H-pyrido[3,4-b]indole; |
| 9 | 6-chloro-7-fluoro-1-(4-fluorophenyl)-2-(4-methylpyrimidin-2-yl)-2,3,4,9-tetrahydro- |
| | 1H-pyrido[3,4-b]indole; |
| 10 | 6-chloro-2-(4-chloropyrimidin-2-yl)-1-(4-methylphenyl)-2,3,4,9-tetrahydro-1H- |
| | pyrido[3,4-b]indole; |
| 11 | 6-chloro-1-(4-chlorophenyl)-7-fluoro-2-(5-fluoropyrimidin-2-yl)-2,3,4,9-tetrahydro- |
| | 1H-pyrido[3,4-b]indole; |
| 12 | 6-chloro-1-(2,4-difluorophenyl)-7-fluoro-2-(5-fluoropyrimidin-2-yl)-2,3,4,9- |
| | tetrahydro-1H-pyrido[3,4-b]indole |
| 13 | 6-chloro-2-(4-chloropyrimidin-2-yl)-1-(4-fluorophenyl)-2,3,4,9-tetrahydro-1H- |
| | pyrido[3,4-b]indole; |
| 14 | 5-[6-chloro-7-fluoro-2-(5-fluoropyrimidin-2-yl)-2,3,4,9-tetrahydro-1H-pyrido[3,4- |
| | b]indol-1-yl]-N,N-dimethylpyrimidin-2-amine; |
| 15 | 6-chloro-2-(4-chloropyrimidin-2-yl)-7-fluoro-1-(4-fluorophenyl)-2,3,4,9-tetrahydro- |
| | 1H-pyrido[3,4-b]indole; |
| 16 | 6-chloro-2-(4,6-dichloropyrimidin-2-yl)-7-fluoro-1-(4-methylphenyl)-2,3,4,9- |
| | tetrahydro-1H-pyrido[3,4-b]indole; |
| 17 | 6-chloro-2-[6-chloro-7-fluoro-1-(4-methylphenyl)-1,3,4,9-tetrahydro-2H-pyrido[3,4- |
| | b]indol-2-yl]-N,N-dimethylpyrimidin-4-amine; |
| 18 | 4-[6-chloro-2-(4-chloropyrimidin-2-yl)-7-fluoro-2,3,4,9-tetrahydro-1H-pyrido[3,4- |
| | b]indol-1-yl]benzonitrile; |
| 19 | 6-chloro-2-(4,6-dimethylpyrimidin-2-yl)-7-fluoro-1-(4-fluorophenyl)-2,3,4,9- |
| | tetrahydro-1H-pyrido[3,4-b]indole; |
| 20 | 6-chloro-5-fluoro-2-(5-fluoropyrimidin-2-yl)-1-(4-methylphenyl)-2,3,4,9-tetrahydro- |
| | 1H-pyrido[3,4-b]indole; |
| 21 | 6-chloro-2-(5-chloropyrimidin-2-yl)-5-fluoro-1-(4-methylphenyl)-2,3,4,9-tetrahydro- |
| | 1H-pyrido[3,4-b]indole; |
| 22 | 6-chloro-2-(4-ethylpyrimidin-2-yl)-7-fluoro-1-(4-fluorophenyl)-2,3,4,9-tetrahydro- |
| | 1H-pyrido[3,4-b]indole; |
| 23 | 6-chloro-7-fluoro-1-(4-fluorophenyl)-2-[4-(propan-2-yl)pyrimidin-2-yl]-2,3,4,9- |
| | tetrahydro-1H-pyrido[3,4-b]indole; |
| 24 | 6-chloro-7-fluoro-1-(4-fluorophenyl)-2-[4-(trifluoromethyl)pyrimidin-2-yl]-2,3,4,9- |
| | tetrahydro-1H-pyrido[3,4-b]indole; |
| 25 | 2-[6-chloro-7-fluoro-1-(4-fluorophenyl)-1,3,4,9-tetrahydro-2H-pyrido[3,4-b]indol-2- |
| | yl]pyrimidine-4-carbonitrile; |
| 26 | 6-chloro-1-(4-chlorophenyl)-5-fluoro-2-(5-fluoropyrimidin-2-yl)-2,3,4,9-tetrahydro- |
| | 1H-pyrido[3,4-b]indole; |
| 27 | 6-chloro-1-(4-chlorophenyl)-2-(5-chloropyrimidin-2-yl)-5-fluoro-2,3,4,9-tetrahydro- |
| | 1H-pyrido[3,4-b]indole; |
| 28 | 6-chloro-2-(4-chloropyrimidin-2-yl)-5-fluoro-1-(4-methylphenyl)-2,3,4,9-tetrahydro- |
| | 1H-pyrido[3,4-b]indole; |
| 29 | 6-chloro-1-(4-chlorophenyl)-2-(5-chloropyrimidin-2-yl)-7-fluoro-2,3,4,9-tetrahydro- |
| | 1H-pyrido[3,4-b]indole; |
| 30 | 6-chloro-7-fluoro-1-(2-fluorophenyl)-2-(5-fluoropyrimidin-2-yl)-2,3,4,9-tetrahydro- |
| | 1H-pyrido[3,4-b]indole; |
| 31 | 6-chloro-1-(4-cyclopropylphenyl)-7-fluoro-2-(5-fluoropyrimidin-2-yl)-2,3,4,9- |
| | tetrahydro-1H-pyrido[3,4-b]indole; |
| 32 | 4-[6-chloro-7-fluoro-2-(5-fluoropyrimidin-2-yl)-2,3,4,9-tetrahydro-1H-pyrido[3,4- |
| | b]indol-1-yl]benzonitrile; |
| 33 | 4-[6-chloro-8-fluoro-2-(5-fluoropyrimidin-2-yl)-2,3,4,9-tetrahydro-1H-pyrido[3,4- |
| | b]indol-1-yl]benzonitrile; |
| 34 | 5-[6-chloro-7-fluoro-2-(5-fluoropyrimidin-2-yl)-2,3,4,9-tetrahydro-1H-pyrido[3,4- |
| | b]indol-1-yl]-N,N-dimethylpyridin-2-amine; |
| 35 | 6-chloro-7-fluoro-2-(5-fluoropyrimidin-2-yl)-1-[4-(trifluoromethyl)phenyl]-2,3,4,9- |
| | tetrahydro-1H-pyrido[3,4-b]indole; |
| 36 | 6-chloro-1-(2,3-difluorophenyl)-7-fluoro-2-(5-fluoropyrimidin-2-yl)-2,3,4,9- |
| | tetrahydro-1H-pyrido[3,4-b]indole; |
| 37 | 6-chloro-1-(3,4-difluorophenyl)-7-fluoro-2-(5-fluoropyrimidin-2-yl)-2,3,4,9- |
| | tetrahydro-1H-pyrido[3,4-b]indole; |
| 38 | 6-chloro-1-(4-chlorophenyl)-7-fluoro-2-(4-methylpyrimidin-2-yl)-2,3,4,9-tetrahydro- |
| | 1H-pyrido[3,4-b]indole; |
| 39 | 6-chloro-1-(4-chlorophenyl)-2-(4-chloropyrimidin-2-yl)-7-fluoro-2,3,4,9-tetrahydro- |
| | 1H-pyrido[3,4-b]indole; |
| 40 | 2-[6-chloro-1-(4-chlorophenyl)-7-fluoro-1,3,4,9-tetrahydro-2H-pyrido[3,4-b]indol-2- |
| | yl]pyrimidine-4-carbonitrile; |
| 41 | 6-chloro-2-(5-chloropyrimidin-2-yl)-7-fluoro-1-(4-methylphenyl)-2,3,4,9-tetrahydro- |
| | 1H-pyrido[3,4-b]indole; |
| 42 | 6-chloro-7-fluoro-1-(4-methylphenyl)-2-(4-methylpyrimidin-2-yl)-2,3,4,9-tetrahydro- |
| | 1H-pyrido[3,4-b]indole; |
| 43 | 6-chloro-2-(4-chloropyrimidin-2-yl)-7-fluoro-1-(4-methylphenyl)-2,3,4,9-tetrahydro- |
| | 1H-pyrido[3,4-b]indole; |
| 44 | 2-[6-chloro-7-fluoro-1-(4-methylphenyl)-1,3,4,9-tetrahydro-2H-pyrido[3,4-b]indol-2- |
| | yl]pyrimidine-4-carbonitrile; |
| 45 | 6-chloro-1-(4-cyclopropylphenyl)-7-fluoro-2-(pyrimidin-2-yl)-2,3,4,9-tetrahydro-1H- |
| | pyrido[3,4-b]indole; |
| 46 | 6-chloro-2-(4-chloro-6-methylpyrimidin-2-yl)-7-fluoro-1-(4-fluorophenyl)-2,3,4,9- |
| | tetrahydro-1H-pyrido[3,4-b]indole; |
| 47 | 6-chloro-7-fluoro-1-(6-fluoropyridin-3-yl)-2-(5-fluoropyrimidin-2-yl)-2,3,4,9- |
| | tetrahydro-1H-pyrido[3,4-b]indole; |
| 48 | 5-[6-chloro-7-fluoro-2-(5-fluoropyrimidin-2-yl)-2,3,4,9-tetrahydro-1H-pyrido[3,4- |
| | b]indol-1-yl]pyridin-2-ol; |
| 49 | 6-chloro-2-(5-chloropyrimidin-2-yl)-5-fluoro-1-(4-fluorophenyl)-2,3,4,9-tetrahydro- |
| | 1H-pyrido[3,4-b]indole; |
| 50 | 6-chloro-7-fluoro-2-(5-fluoropyrimidin-2-yl)-1-[4-(propan-2-yl)phenyl]-2,3,4,9- |
| | tetrahydro-1H-pyrido[3,4-b]indole; |
| 51 | 6-chloro-5-fluoro-1-(4-fluorophenyl)-2-(5-fluoropyrimidin-2-yl)-2,3,4,9-tetrahydro- |
| | 1H-pyrido[3,4-b]indole; |
| 52 | 4-[6-chloro-2-(5-chloropyrimidin-2-yl)-5-fluoro-2,3,4,9-tetrahydro-1H-pyrido[3,4- |
| | b]indol-1-yl]benzonitrile; |
| 53 | 6-chloro-2-(4-chloropyrimidin-2-yl)-7-fluoro-1-[4-(propan-2-yl)phenyl]-2,3,4,9- |
| | tetrahydro-1H-pyrido[3,4-b]indole; |
| 54 | 6-chloro-7-fluoro-2-(4-methylpyrimidin-2-yl)-1-[4-(propan-2-yl)phenyl]-2,3,4,9- |
| | tetrahydro-1H-pyrido[3,4-b]indole; |
| 55 | 2-{6-chloro-7-fluoro-1-[4-(propan-2-yl)phenyl]-1,3,4,9-tetrahydro-2H-pyrido[3,4- |
| | b]indol-2-yl}pyrimidine-4-carbonitrile; |
| 56 | 4-[6-chloro-5-fluoro-2-(5-fluoropyrimidin-2-yl)-2,3,4,9-tetrahydro-1H-pyrido[3,4- |
| | b]indol-1-yl]benzonitrile; |
| 57 | 6-chloro-2-(5-chloropyrimidin-2-yl)-7-fluoro-1-[4-(propan-2-yl)phenyl]-2,3,4,9- |
| | tetrahydro-1H-pyrido[3,4-b]indole; |
| 58 | 6-chloro-7-fluoro-1-(3-fluorophenyl)-2-(5-fluoropyrimidin-2-yl)-2,3,4,9-tetrahydro- |
| | 1H-pyrido[3,4-b]indole; |
| 59 | 4-[6-chloro-2-(5-chloropyrimidin-2-yl)-7-fluoro-2,3,4,9-tetrahydro-1H-pyrido[3,4- |
| | b]indol-1-yl]benzonitrile; |
| 60 | 6-chloro-5-fluoro-1-(4-fluorophenyl)-2-[4-(trifluoromethyl)pyrimidin-2-yl]-2,3,4,9- |
| | tetrahydro-1H-pyrido[3,4-b]indole; |
| 61 | 4-[6-chloro-7-fluoro-2-(4-methylpyrimidin-2-yl)-2,3,4,9-tetrahydro-1H-pyrido[3,4- |
| | b]indol-1-yl]benzonitrile; |
| 62 | 6-chloro-2-(5-chloropyrimidin-2-yl)-5-fluoro-1-[4-(trifluoromethyl)phenyl]-2,3,4,9- |
| | tetrahydro-1H-pyrido[3,4-b]indole; |
| 63 | 6-chloro-1-(2,3-difluorophenyl)-7-fluoro-2-(4-methylpyrimidin-2-yl)-2,3,4,9- |
| | tetrahydro-1H-pyrido[3,4-b]indole; |
| 64 | 2-[6-chloro-1-(4-cyanophenyl)-7-fluoro-1,3,4,9-tetrahydro-2H-pyrido[3,4-b]indol-2- |
| | yl]pyrimidine-4-carbonitrile; |
| 65 | {4-[6-chloro-2-(5-fluoropyrimidin-2-yl)-2,3,4,9-tetrahydro-1H-pyrido[3,4-b]indol-1- |
| | yl]phenyl}methanol; |
| 66 | 6-chloro-2-(4,6-dimethylpyrimidin-2-yl)-7-fluoro-1-(4-methylphenyl)-2,3,4,9- |
| | tetrahydro-1H-pyrido[3,4-b]indole; |
| 67 | 6-chloro-5-fluoro-2-(5-fluoropyrimidin-2-yl)-1-[4-(trifluoromethyl)phenyl]-2,3,4,9- |
| | tetrahydro-1H-pyrido[3,4-b]indole; |
| 68 | 2-[6-chloro-1-(2,3-difluorophenyl)-7-fluoro-1,3,4,9-tetrahydro-2H-pyrido[3,4-b]indol- |
| | 2-yl]pyrimidine-4-carbonitrile; |
| 69 | 4-[2-(4-bromopyrimidin-2-yl)-6-chloro-7-fluoro-2,3,4,9-tetrahydro-1H-pyrido[3,4- |
| | b]indol-1-yl]benzonitrile; |
| 70 | 6-chloro-1-(4-chlorophenyl)-5-fluoro-2-(5-fluoro-4-methylpyrimidin-2-yl)-2,3,4,9- |
| | tetrahydro-1H-pyrido[3,4-b]indole; |
| 71 | 6-chloro-1-(4-ethenylphenyl)-2-(5-fluoropyrimidin-2-yl)-2,3,4,9-tetrahydro-1H- |
| | pyrido[3,4-b]indole; |
| 72 | 6-chloro-7-fluoro-1-(4-methylphenyl)-2-[4-(trifluoromethyl)pyrimidin-2-yl]-2,3,4,9- |
| | tetrahydro-1H-pyrido[3,4-b]indole; |
| 73 | 6-chloro-1-(4-chlorophenyl)-5-fluoro-2-[4-(trifluoromethyl)pyrimidin-2-yl]-2,3,4,9- |
| | tetrahydro-1H-pyrido[3,4-b]indole; |
| 74 | 6-chloro-5-fluoro-1-[4-(trifluoromethyl)phenyl]-2-[4-(trifluoromethyl)pyrimidin-2- |
| | yl]-2,3,4,9-tetrahydro-1H-pyrido[3,4-b]indole; |
| 75 | 6-chloro-7-fluoro-1-[4-(trifluoromethyl)phenyl]-2-[4-(trifluoromethyl)pyrimidin-2- |
| | yl]-2,3,4,9-tetrahydro-1H-pyrido[3,4-b]indole; |
| 76 | 6-chloro-5-fluoro-1-(4-methylphenyl)-2-[4-(trifluoromethyl)pyrimidin-2-yl]-2,3,4,9- |
| | tetrahydro-1H-pyrido[3,4-b]indole; |
| 77 | 6-chloro-2-(4-chloropyrimidin-2-yl)-1-(2,3-difluorophenyl)-7-fluoro-2,3,4,9- |
| | tetrahydro-1H-pyrido[3,4-b]indole; |
| 78 | {4-[6-chloro-7-fluoro-2-(5-fluoropyrimidin-2-yl)-2,3,4,9-tetrahydro-1H-pyrido[3,4- |
| | b]indol-1-yl]phenyl}methanol; |
| 79 | 6-chloro-1-(4-ethenylphenyl)-7-fluoro-2-(5-fluoropyrimidin-2-yl)-2,3,4,9-tetrahydro- |
| | 1H-pyrido[3,4-b]indole; |
| 80 | 6-chloro-2-(5-chloropyrimidin-2-yl)-7-fluoro-1-[4-(trifluoromethyl)phenyl]-2,3,4,9- |
| | tetrahydro-1H-pyrido[3,4-b]indole; |
| 81 | 6-chloro-7-fluoro-2-(4-methylpyrimidin-2-yl)-1-[4-(trifluoromethyl)phenyl]-2,3,4,9- |
| | tetrahydro-1H-pyrido[3,4-b]indole; |
| 82 | 5,6-difluoro-2-(5-fluoropyrimidin-2-yl)-1-(4-methylphenyl)-2,3,4,9-tetrahydro-1H- |
| | pyrido[3,4-b]indole; |
| 83 | 5,6-difluoro-1-(4-fluorophenyl)-2-(5-fluoropyrimidin-2-yl)-2,3,4,9-tetrahydro-1H- |
| | pyrido[3,4-b]indole; |
| 84 | 2-(5-chloropyrimidin-2-yl)-5,6-difluoro-1-(4-methylphenyl)-2,3,4,9-tetrahydro-1H- |
| | pyrido[3,4-b]indole; |
| 85 | 2-(5-chloropyrimidin-2-yl)-5,6-difluoro-1-(4-fluorophenyl)-2,3,4,9-tetrahydro-1H- |
| | pyrido[3,4-b]indole; |
| 86 | 2-{4-[6-chloro-7-fluoro-2-(5-fluoropyrimidin-2-yl)-2,3,4,9-tetrahydro-1H-pyrido[3,4- |
| | b]indol-1-yl]phenyl}ethan-1-ol; |
| 87 | methyl 2-[6-chloro-1-(4-chlorophenyl)-5-fluoro-1,3,4,9-tetrahydro-2H-pyrido[3,4- |
| | b]indol-2-yl]pyrimidine-4-carboxylate; |
| 88 | 4-{6-chloro-7-fluoro-2-[4-methyl-6-(trifluoromethyl)pyrimidin-2-yl]-2,3,4,9- |
| | tetrahydro-1H-pyrido[3,4-b]indol-1-yl}benzonitrile; |
| 89 | 4-{6-chloro-5-fluoro-2-[4-(trifluoromethyl)pyrimidin-2-yl]-2,3,4,9-tetrahydro-1H- |
| | pyrido[3,4-b]indol-1-yl}benzonitrile; |
| 90 | 6-chloro-5-fluoro-1-(4-fluorophenyl)-2-(4-methylpyrimidin-2-yl)-2,3,4,9-tetrahydro- |
| | 1H-pyrido[3,4-b]indole; |
| 91 | 6-chloro-7-fluoro-2-(5-fluoropyrimidin-2-yl)-1-(2-methylpyrimidin-5-yl)-2,3,4,9- |
| | tetrahydro-1H-pyrido[3,4-b]indole; |
| 92 | 6-chloro-7-fluoro-1-(2-methylpyrimidin-5-yl)-2-[4-(trifluoromethyl)pyrimidin-2-yl]- |
| | 2,3,4,9-tetrahydro-1H-pyrido[3,4-b]indole; |
| 93 | 4-[6-chloro-2-(4,6-dimethylpyrimidin-2-yl)-7-fluoro-2,3,4,9-tetrahydro-1H- |
| | pyrido[3,4-b]indol-1-yl]benzonitrile; |
| 94 | 5-[6-chloro-7-fluoro-2-(4-methylpyrimidin-2-yl)-2,3,4,9-tetrahydro-1H-pyrido[3,4- |
| | b]indol-1-yl]-N,N-dimethylpyrimidin-2-amine; |
| 95 | 5-{6-chloro-7-fluoro-2-[4-(trifluoromethyl)pyrimidin-2-yl]-2,3,4,9-tetrahydro-1H- |
| | pyrido[3,4-b]indol-1-yl}-N,N-dimethylpyrimidin-2-amine; |
| 96 | 6-chloro-7-fluoro-1-(4-methylphenyl)-2-[4-methyl-6-(trifluoromethyl)pyrimidin-2- |
| | yl]-2,3,4,9-tetrahydro-1H-pyrido[3,4-b]indole; |
| 97 | 6-chloro-7-fluoro-1-(4-methylphenyl)-2-[5-(trifluoromethyl)pyrimidin-2-yl]-2,3,4,9- |
| | tetrahydro-1H-pyrido[3,4-b]indole; |
| 98 | 6-chloro-7-fluoro-1-(4-fluorophenyl)-2-[S-(trifluoromethyl)pyrimidin-2-yl]-2,3,4,9- |
| | tetrahydro-1H-pyrido[3,4-b]indole; |
| 99 | 6-chloro-7-fluoro-1-[4-(propan-2-yl)phenyl]-2-[5-(trifluoromethyl)pyrimidin-2-yl]- |
| | 2,3,4,9-tetrahydro-1H-pyrido[3,4-b]indole; |
| 100 | 4-{6-chloro-7-fluoro-2-[4-(trifluoromethyl)pyrimidin-2-yl]-2,3,4,9-tetrahydro-1H- |
| | pyrido[3,4-b]indol-1-yl}benzonitrile, |
| 101 | 6-chloro-2-(5-chloropyrimidin-2-yl)-7-fluoro-1-(2-methylpyrimidin-5-yl)-2,3,4,9- |
| | tetrahydro-1H-pyrido[3,4-b]indole; |
| 102 | 5-[6-chloro-2-(5-chloropyrimidin-2-yl)-7-fluoro-2,3,4,9-tetrahydro-1H-pyrido[3,4- |
| | b]indol-1-yl]-N,N-dimethylpyrimidin-2-amine; |
| 103 | 6-chloro-7-fluoro-1-[4-(methanesulfonyl)phenyl]-2-[4-(trifluoromethyl)pyrimidin-2- |
| | yl]-2,3,4,9-tetrahydro-1H-pyrido[3,4-b]indole; |
| 104 | methyl 2-[6-chloro-1-(4-cyanophenyl)-5-fluoro-1,3,4,9-tetrahydro-2H-pyrido[3,4- |
| | b]indol-2-yl]pyrimidine-4-carboxylate; |
| 105 | 6-chloro-7-fluoro-2-(5-fluoro-4-methylpyrimidin-2-yl)-1-(4-fluorophenyl)-2,3,4,9- |
| | tetrahydro-1H-pyrido[3,4-b]indole; |
| 106 | 6-chloro-7-fluoro-2-(5-fluoro-4,6-dimethylpyrimidin-2-yl)-1-(4-methylphenyl)- |
| | 2,3,4,9-tetrahydro-1H-pyrido[3,4-b]indole; |
| 107 | 6-chloro-7-fluoro-2-(5-fluoropyrimidin-2-yl)-1-[4-(methanesulfonyl)phenyl]-2,3,4,9- |
| | tetrahydro-1H-pyrido[3,4-b]indole; |
| 108 | 6-chloro-1-(2,3-difluorophenyl)-7-fluoro-2-[4-(trifluoromethyl)pyrimidin-2-yl]- |
| | 2,3,4,9-tetrahydro-1H-pyrido[3,4-b]indole; |
| 109 | 6-chloro-5-fluoro-2-(5-fluoro-4-methylpyrimidin-2-yl)-1-(4-methylphenyl)-2,3,4,9- |
| | tetrahydro-1H-pyrido[3,4-b]indole; |
| 110 | 2-[6-chloro-5-fluoro-1-(4-methylphenyl)-1,3,4,9-tetrahydro-2H-pyrido[3,4-b]indol-2- |
| | yl]-6-methylpyrimidine-4-carbonitrile; |
| 111 | 2-[6-chloro-5-fluoro-1-(4-fluorophenyl)-1,3,4,9-tetrahydro-2H-pyrido[3,4-b]indol-2- |
| | yl]-6-methylpyrimidine-4-carbonitrile; |
| 112 | 6-chloro-1-(2,3-difluorophenyl)-7-fluoro-2-(5-fluoro-4,6-dimethylpyrimidin-2-yl)- |
| | 2,3,4,9-tetrahydro-1H-pyrido[3,4-b]indole; |
| 113 | 6-chloro-1-(2,3-difluorophenyl)-5-fluoro-2-(5-fluoro-4-methylpyrimidin-2-yl)-2,3,4,9- |
| | tetrahydro-1H-pyrido[3,4-b]indole; |
| 114 | 6-chloro-1-(2,3-difluoro-4-methylphenyl)-5-fluoro-2-(5-fluoro-4-methylpyrimidin-2- |
| | yl)-2,3,4,9-tetrahydro-1H-pyrido[3,4-b]indole |
| 115 | 4-{6-chloro-7-fluoro-2-[4-(trifluoromethyl)pyrimidin-2-yl]-2,3,4,9-tetrahydro-1H- |
| | pyrido[3,4-b]indol-1-yl}benzene-1-sulfonamide; |
| 116 | 6-chloro-5-fluoro-2-(5-fluoro-4,6-dimethylpyrimidin-2-yl)-1-(4-methylphenyl)- |
| | 2,3,4,9-tetrahydro-1H-pyrido[3,4-b]indole; |
| 117 | 6-chloro-1-(2,3-difluorophenyl)-5-fluoro-2-(5-fluoro-4,6-dimethylpyrimidin-2-yl)- |
| | 2,3,4,9-tetrahydro-1H-pyrido[3,4-b]indole; |
| 118 | 6-chloro-1-(2,3-difluoro-4-methylphenyl)-5-fluoro-2-(5-fluoro-4,6- |
| | dimethylpyrimidin-2-yl)-2,3,4,9-tetrahydro-1H-pyrido[3,4-b]indole; |
| 119 | 6-chloro-1-(2,3-difluorophenyl)-2-[4-(trifluoromethyl)pyrimidin-2-yl]-2,3,4,9- |
| | tetrahydro-1H-pyrido[3,4-b]indole; |
| 120 | 6-chloro-1-(2,3-difluoro-4-methoxyphenyl)-2-[4-(trifluoromethyl)pyrimidin-2-yl]- |
| | 2,3,4,9-tetrahydro-1H-pyrido[3,4-b]indole; |
| 121 | 2-{6-chloro-7-fluoro-1-[4-(trifluoromethyl)phenyl]-1,3,4,9-tetrahydro-2H-pyrido[3,4- |
| | b]indol-2-yl}pyrimidine-4-carbonitrile; |
| 122 | 5-(6-chloro-7-fluoro-2-[4-(trifluoromethyl)pyrimidin-2-yl]-2,3,4,9-tetrahydro-1H- |
| | pyrido[3,4-b]indol-1-yl}-N,N-dimethylpyridin-2-amine; |
| 123 | 6-chloro-7-fluoro-2-(5-fluoro-4-methylpyrimidin-2-yl)-1-(4-methylphenyl)-2,3,4,9- |
| | tetrahydro-1H-pyrido[3,4-b]indole; |
| 124 | 6-chloro-1-(2,3-difluorophenyl)-7-fluoro-2-(5-fluoro-4-methylpyrimidin-2-yl)-2,3,4,9- |
| | tetrahydro-1H-pyrido[3,4-b]indole; |
| 125 | 6-chloro-1-(2,3-difluoro-4-methylphenyl)-7-fluoro-2-(5-fluoro-4-methylpyrimidin-2- |
| | yl)-2,3,4,9-tetrahydro-1H-pyrido[3,4-b]indole; |
| 126 | 5-[6-chloro-7-fluoro-2-(4-methylpyrimidin-2-yl)-2,3,4,9-tetrahydro-1H-pyrido[3,4- |
| | b]indol-1-yl]-N,N-dimethylpyridin-2-amine; |
| 127 | 4-[6-chloro-7-fluoro-2-(5-fluoropyrimidin-2-yl)-2,3,4,9-tetrahydro-1H-pyrido[3,4- |
| | b]indol-1-yl]benzene-1-sulfonamide; |
| 128 | 6-chloro-1-(2,3-difluoro-4-methylphenyl)-2-[4-(trifluoromethyl)pyrimidin-2-yl]- |
| | 2,3,4,9-tetrahydro-1H-pyrido[3,4-b]indole; |
| 129 | 6-chloro-1-(2,3-difluorophenyl)-2-(4,6-dimethylpyrimidin-2-yl)-7-fluoro-2,3,4,9- |
| | tetrahydro-1H-pyrido[3,4-b]indole; |
| 130 | 6-chloro-2-(5-chloropyrimidin-2-yl)-1-(2,3-difluoro-4-methylphenyl)-7-fluoro- |
| | 2,3,4,9-tetrahydro-1H-pyrido[3,4-b]indole; |
| 131 | 6-chloro-2-(5-chloropyrimidin-2-yl)-1-(2,3-difluorophenyl)-7-fluoro-2,3,4,9- |
| | tetrahydro-1H-pyrido[3,4-b]indole; |
| 132 | 6-chloro-1-(2,3-difluoro-4-methylphenyl)-2-(4,6-dimethylpyrimidin-2-yl)-7-fluoro- |
| | 2,3,4,9-tetrahydro-1H-pyrido[3,4-b]indole; |
| 133 | 6-chloro-1-(2,3-difluorophenyl)-7-fluoro-2-[5-(trifluoromethyl)pyrimidin-2-yl]- |
| | 2,3,4,9-tetrahydro-1H-pyrido[3,4-b]indole; |
| 134 | 6-chloro-7-fluoro-1-(4-methylphenyl)-2-(5-methylpyrimidin-2-yl)-2,3,4,9-tetrahydro- |
| | 1H-pyrido[3,4-b]indole; |
| 135 | 6-chloro-5-fluoro-1-(4-methylphenyl)-2-(5-methylpyrimidin-2-yl)-2,3,4,9-tetrahydro- |
| | 1H-pyrido[3,4-b]indole; |
| 136 | 6-chloro-1-(2,3-difluoro-4-methylphenyl)-7-fluoro-2-(5-methylpyrimidin-2-yl)- |
| | 2,3,4,9-tetrahydro-1H-pyrido[3,4-b]indole; |
| 137 | 6-chloro-1-(2,3-difluoro-4-methylphenyl)-5-fluoro-2-(5-methylpyrimidin-2-yl)- |
| | 2,3,4,9-tetrahydro-1H-pyrido[3,4-b]indole; |
| 138 | 6-chloro-1-(2,3-difluoro-4-methylphenyl)-7-fluoro-2-(5-fluoropyrimidin-2-yl)-2,3,4,9- |
| | tetrahydro-1H-pyrido[3,4-b]indole; |
| 139 | 6-chloro-1-(2,3-difluoro-4-methylphenyl)-7-fluoro-2-(4-methylpyrimidin-2-yl)- |
| | 2,3,4,9-tetrahydro-1H-pyrido[3,4-b]indole; |
| 140 | 6-chloro-1-(2,3-difluoro-4-methylphenyl)-7-fluoro-2-[4-(trifluoromethyl)pyrimidin-2- |
| | yl]-2,3,4,9-tetrahydro-1H-pyrido[3,4-b]indole; |
| 141 | 2-[6-chloro-1-(2,3-difluoro-4-methylphenyl)-7-fluoro-1,3,4,9-tetrahydro-2H- |
| | pyrido[3,4-b]indol-2-yl]pyrimidine-4-carbonitrile; |
| 142 | 6-chloro-1-(2,3-difluoro-4-methylphenyl)-7-fluoro-2-[5-(trifluoromethyl)pyrimidin-2- |
| | yl]-2,3,4,9-tetrahydro-1H-pyrido[3,4-b]indole; |
| 143 | 6-chloro-1-(2,3-difluoro-4-methylphenyl)-7-fluoro-2-(5-fluoro-4,6- |
| | dimethylpyrimidin-2-yl)-2,3,4,9-tetrahydro-1H-pyrido[3,4-b]indole; |
| 144 | 6-chloro-1-(4-chloro-2,3-difluorophenyl)-2-[4-(trifluoromethyl)pyrimidin-2-yl]- |
| | 2,3,4,9-tetrahydro-1H-pyrido[3,4-b]indole; |
| 145 | 6-chloro-7-fluoro-1-(5-fluoropyridin-3-yl)-2-[4-(trifluoromethyl)pyrimidin-2-yl]- |
| | 2,3,4,9-tetrahydro-1H-pyrido[3,4-b]indole; |
| 146 | 6-chloro-5-fluoro-1-(5-fluoropyridin-3-yl)-2-[4-(trifluoromethyl)pyrimidin-2-yl]- |
| | 2,3,4,9-tetrahydro-1H-pyrido[3,4-b]indole; |
| 147 | 6-chloro-1-(2,3-difluorophenyl)-7-fluoro-2-[4-methyl-6-(trifluoromethyl)pyrimidin-2- |
| | yl]-2,3,4,9-tetrahydro-1H-pyrido[3,4-b]indole; |
| 148 | 6-chloro-1-(2,3-difluoro-4-methylphenyl)-7-fluoro-2-[4-methyl-6- |
| | (trifluoromethyl)pyrimidin-2-yl]-2,3,4,9-tetrahydro-1H-pyrido[3,4-b]indole; |
| 149 | 6-chloro-5-fluoro-1-[4-(methanesulfonyl)phenyl]-2-[4-(trifluoromethyl)pyrimidin-2- |
| | yl]-2,3,4,9-tetrahydro-1H-pyrido[3,4-b]indole; |
| 150 | 4-{6-chloro-5-fluoro-2-[4-(trifluoromethyl)pyrimidin-2-yl]-2,3,4,9-tetrahydro-1H- |
| | pyrido[3,4-b]indol-1-yl}benzene-1-sulfonamide; |
| 151 | 2-[6-chloro-7-fluoro-1-(4-methylphenyl)-1,3,4,9-tetrahydro-2H-pyrido[3,4-b]indol-2- |
| | yl]-6-methylpyrimidine-4-carbonitrile; |
| 152 | 2-[6-chloro-1-(2,3-difluorophenyl)-7-fluoro-1,3,4,9-tetrahydro-2H-pyrido[3,4-b]indol- |
| | 2-yl]-6-methylpyrimidine-4-carbonitrile; |
| 153 | 2-[6-chloro-1-(2,3-difluoro-4-methylphenyl)-7-fluoro-1,3,4,9-tetrahydro-2H- |
| | pyrido[3,4-b]indol-2-yl]-6-methylpyrimidine-4-carbonitrile; |
| 154 | 2-[4,6-bis(trifluoromethyl)pyrimidin-2-yl]-6-chloro-7-fluoro-1-(4-methylphenyl)- |
| | 2,3,4,9-tetrahydro-1H-pyrido[3,4-b]indole; |
| 155 | 2-[4,6-bis(trifluoromethyl)pyrimidin-2-yl]-6-chloro-1-(2,3-difluoro-4-methylphenyl)- |
| | 7-fluoro-2,3,4,9-tetrahydro-1H-pyrido[3,4-b]indole; |
| 156 | 3-(4-{6-chloro-7-fluoro-2-[4-(trifluoromethyl)pyrimidin-2-yl]-2,3,4,9-tetrahydro-1H- |
| | pyrido[3,4-b]indol-1-yl}phenyl)propan-1-ol; |
| 157 | 2-[4,6-bis(trifluoromethyl)pyrimidin-2-yl]-6-chloro-1-(2,3-difluorophenyl)-7-fluoro- |
| | 2,3,4,9-tetrahydro-1H-pyrido[3,4-b]indole; |
| 158 | 5-{6-chloro-7-fluoro-2-[4-(trifluoromethyl)pyrimidin-2-yl]-2,3,4,9-tetrahydro-1H- |
| | pyrido[3,4-b]indol-1-yl}-3-fluoro-N,N-dimethylpyridin-2-amine; |
| 159 | 3-{4-[6-chloro-7-fluoro-2-(5-fluoropyrimidin-2-yl)-2,3,4,9-tetrahydro-1H-pyrido[3,4- |
| | b]indol-1-yl]phenyl}propan-1-ol; |
| 160 | 5-{6-chloro-5-fluoro-2-[4-(trifluoromethyl)pyrimidin-2-yl]-2,3,4,9-tetrahydro-1H- |
| | pyrido[3,4-b]indol-1-yl}-3-fluoro-N,N-dimethylpyridin-2-amine; |
| 161 | (1S)-6-chloro-1-(4-methylphenyl)-2-[4-(trifluoromethyl)pyrimidin-2-yl]-2,3,4,9- |
| | tetrahydro-1H-pyrido[3,4-b]indole; |
| 162 | 2-[6-chloro-1-(2,3-difluoro-4-methylphenyl)-7-fluoro-1,3,4,9-tetrahydro-2H- |
| | pyrido[3,4-b]indol-2-yl]-5-fluoropyrimidine-4-carbonitrile; |
| 163 | 6-chloro-7-fluoro-1-(4-methylphenyl)-2-(4,5,6-trimethylpyrimidin-2-yl)-2,3,4,9- |
| | tetrahydro-1H-pyrido[3,4-b]indole; |
| 164 | methyl 3-(4-{6-chloro-7-fluoro-2-[4-(trifluoromethyl)pyrimidin-2-yl]-2,3,4,9- |
| | tetrahydro-1H-pyrido[3,4-b]indol-1-yl}phenyl)propanoate; |
| 165 | methyl 3-{4-[6-chloro-7-fluoro-2-(5-fluoropyrimidin-2-yl)-2,3,4,9-tetrahydro-1H- |
| | pyrido[3,4-b]indol-1-yl]phenyl}propanoate; |
| 166 | (4-{6-chloro-7-fluoro-2-[4-(trifluoromethyl)pyrimidin-2-yl]-2,3,4,9-tetrahydro-1H- |
| | pyrido[3,4-b]indol-1-yl}phenyl)methanol; |
| 167 | (3E)-4-(4-{6-chloro-7-fluoro-2-[4-(trifluoromethyl)pyrimidin-2-yl]-2,3,4,9- |
| | tetrahydro-1H-pyrido[3,4-b]indol-1-yl}phenyl)but-3-en-1-ol; |
| 168 | 4-(4-{6-chloro-7-fluoro-2-[4-(trifluoromethyl)pyrimidin-2-yl]-2,3,4,9-tetrahydro-1H- |
| | pyrido[3,4-b]indol-1-yl}phenyl)butan-1-ol; |
| 169 | 6-chloro-1-(2,3-difluoro-4-methylphenyl)-7-fluoro-2-(4,5,6-trimethylpyrimidin-2-yl)- |
| | 2,3,4,9-tetrahydro-1H-pyrido[3,4-b]indole; |
| 170 | 4-{6-chloro-7-fluoro-2-[4-(trifluoromethyl)pyrimidin-2-yl]-2,3,4,9-tetrahydro-1H- |
| | pyrido[3,4-b]indol-1-yl}-N,N-dimethylaniline; |
| 171 | 6-chloro-1-(2,3-difluorophenyl)-7-fluoro-2-(4,5,6-trimethylpyrimidin-2-yl)-2,3,4,9- |
| | tetrahydro-1H-pyrido[3,4-b]indole; |
| 172 | 1-(4-bromo-2,3-difluorophenyl)-6-chloro-7-fluoro-2-[4-(trifluoromethyl)pyrimidin-2- |
| | yl]-2,3,4,9-tetrahydro-1H-pyrido[3,4-b]indole; |
| 173 | 4-{6-chloro-7-fluoro-2-[4-(trifluoromethyl)pyrimidin-2-yl]-2,3,4,9-tetrahydro-1H- |
| | pyrido[3,4-b]indol-1-yl}-2,3-difluoro-N,N-dimethylaniline; |
| 174 | 5-fluoro-6-methyl-1-(4-methylphenyl)-2-[4-(trifluoromethyl)pyrimidin-2-yl]-2,3,4,9- |
| | tetrahydro-1H-pyrido[3,4-b]indole; |
| 175 | 2-[6-chloro-7-fluoro-1-(4-methylphenyl)-1,3,4,9-tetrahydro-2H-pyrido[3,4-b]indol-2- |
| | yl]-5-fluoropyrimidine-4-carbonitrile; |
| 176 | 2-[(1R)-6-chloro-1-(2,3-difluoro-4-methylphenyl)-1,3,4,9-tetrahydro-2H-pyrido[3,4- |
| | b]indol-2-yl]pyrimidine-4-carbonitrile; |
| 177 | 5-{6-chloro-5-fluoro-2-[4-(trifluoromethyl)pyrimidin-2-yl]-2,3,4,9-tetrahydro-1H- |
| | pyrido[3,4-b]indol-1-yl}-N,N-dimethylpyridin-2-amine; |
| 178 | 5-{6-chloro-5-fluoro-2-[4-(trifluoromethyl)pyrimidin-2-yl]-2,3,4,9-tetrahydro-1H- |
| | pyrido[3,4-b]indol-1-yl}-N,N-dimethylpyrimidin-2-amine; |
| 179 | (1R)-6-chloro-1-(2,3-difluoro-4-methylphenyl)-2-(5-fluoropyrimidin-2-yl)-2,3,4,9- |
| | tetrahydro-1H-pyrido[3,4-b]indole; |
| 180 | (1R)-6-chloro-1-(2,3-difluoro-4-methylphenyl)-2-[4-(trifluoromethyl)pyrimidin-2-yl]- |
| | 2,3,4,9-tetrahydro-1H-pyrido[3,4-b]indole; |
| 181 | (1R)-6-chloro-1-(4-chlorophenyl)-5-fluoro-2-[4-(trifluoromethyl)pyrimidin-2-yl]- |
| | 2,3,4,9-tetrahydro-1H-pyrido[3,4-b]indole; |
| 182 | (1S)-6-chloro-1-(4-chlorophenyl)-5-fluoro-2-[4-(trifluoromethyl)pyrimidin-2-yl]- |
| | 2,3,4,9-tetrahydro-1H-pyrido[3,4-b]indole; |
| 183 | 2-[6-chloro-1-(2,3-difluorophenyl)-7-fluoro-1,3,4,9-tetrahydro-2H-pyrido[3,4-b]indol- |
| | 2-yl]-5-fluoropyrimidine-4-carbonitrile; |
| 184 | 6-chloro-1-(2,6-difluoropyridin-3-yl)-7-fluoro-2-[4-(trifluoromethyl)pyrimidin-2-yl]- |
| | 2,3,4,9-tetrahydro-1H-pyrido[3,4-b]indole; |
| 185 | (1R)-6-chloro-1-(2,3-difluoro-4-methylphenyl)-2-(4-methylpyrimidin-2-yl)-2,3,4,9- |
| | tetrahydro-1H-pyrido[3,4-b]indole; |
| 186 | 6-chloro-1-(2,6-difluoropyridin-3-yl)-5-fluoro-2-[4-(trifluoromethyl)pyrimidin-2-yl]- |
| | 2,3,4,9-tetrahydro-1H-pyrido[3,4-b]indole; |
| 187 | (1R)-6-chloro-1-(2,3-difluoro-4-methylphenyl)-2-(5-fluoro-4-methylpyrimidin-2-yl)- |
| | 2,3,4,9-tetrahydro-1H-pyrido[3,4-b]indole; |
| 188 | 4-(2-[4,6-bis(trifluoromethyl)pyrimidin-2-yl]-6-chloro-7-fluoro-2,3,4,9-tetrahydro- |
| | 1H-pyrido[3,4-b]indol-1-yl}benzonitrile; |
| 189 | (1S)-6-chloro-1-(2,3-difluoro-4-methylphenyl)-5-fluoro-2-(5-fluoro-4,6- |
| | dimethylpyrimidin-2-yl)-2,3,4,9-tetrahydro-1H-pyrido[3,4-b]indole; |
| 190 | (1R)-6-chloro-1-(2,3-difluoro-4-methylphenyl)-5-fluoro-2-(5-fluoro-4,6- |
| | dimethylpyrimidin-2-yl)-2,3,4,9-tetrahydro-1H-pyrido[3,4-b]indole; |
| 191 | 6-chloro-5-fluoro-1-(pyrimidin-5-yl)-2-[4-(trifluoromethyl)pyrimidin-2-yl]-2,3,4,9- |
| | tetrahydro-1H-pyrido[3,4-b]indole; |
| 192 | 1-(2,3-difluoro-4-methylphenyl)-5-fluoro-6-methyl-2-[4-(trifluoromethyl)pyrimidin-2- |
| | yl]-2,3,4,9-tetrahydro-1H-pyrido[3,4-b]indole; |
| 193 | 6-chloro-5-fluoro-1-[2-(1H-imidazol-1-yl)pyrimidin-5-yl]-2-[4- |
| | (trifluoromethyl)pyrimidin-2-yl]-2,3,4,9-tetrahydro-1H-pyrido[3,4-b]indole; |
| 194 | 5-[6-chloro-5-fluoro-2-(5-fluoropyrimidin-2-yl)-2,3,4,9-tetrahydro-1H-pyrido[3,4- |
| | b]indol-1-yl]-N,N-dimethylpyridin-2-amine; |
| 195 | 6-chloro-2-(5-chloropyrimidin-2-yl)-1-(2,3-difluoro-4-methylphenyl)-5-fluoro- |
| | 2,3,4,9-tetrahydro-1H-pyrido[3,4-b]indole; |
| 196 | 6-chloro-1-(2,3-difluoro-4-methylphenyl)-5-fluoro-2-(4-methylpyrimidin-2-yl)- |
| | 2,3,4,9-tetrahydro-1H-pyrido[3,4-b]indole; |
| 197 | 6-chloro-1-(2,3-difluoro-4-methylphenyl)-5-fluoro-2-[4-(trifluoromethyl)pyrimidin-2- |
| | yl]-2,3,4,9-tetrahydro-1H-pyrido[3,4-b]indole; |
| 198 | 2-[6-chloro-1-(2,3-difluoro-4-methylphenyl)-5-fluoro-1,3,4,9-tetrahydro-2H- |
| | pyrido[3,4-b]indol-2-yl]pyrimidine-4-carbonitrile; |
| 199 | 5-{6-chloro-5-fluoro-2-[4-(trifluoromethyl)pyrimidin-2-yl]-2,3,4,9-tetrahydro-1H- |
| | pyrido[3,4-b]indol-1-yl}-N-methylpyrimidin-2-amine; |
| 200 | 6-chloro-5-fluoro-1-[2-(morpholin-4-yl)pyrimidin-5-yl]-2-[4- |
| | (trifluoromethyl)pyrimidin-2-yl]-2,3,4,9-tetrahydro-1H-pyrido[3,4-b]indole; |
| 201 | 5-[6-chloro-5-fluoro-2-(5-fluoropyrimidin-2-yl)-2,3,4,9-tetrahydro-1H-pyrido[3,4- |
| | b]indol-1-yl]-N,N-dimethylpyrimidin-2-amine; |
| 202 | 6-chloro-5-fluoro-1-(pyridin-3-yl)-2-[4-(trifluoromethyl)pyrimidin-2-yl]-2,3,4,9- |
| | tetrahydro-1H-pyrido[3,4-b]indole; |
| 203 | 6-chloro-1-(2,3-difluoro-4-methylphenyl)-2-(4-methylpyrimidin-2-yl)-2,3,4,9- |
| | tetrahydro-1H-pyrido[3,4-b]indole; |
| 204 | (1R)-6-chloro-1-(2,3-difluoro-4-methylphenyl)-2-[4-methyl-6- |
| | (trifluoromethyl)pyrimidin-2-yl]-2,3,4,9-tetrahydro-1H-pyrido[3,4-b]indole; |
| 205 | 6-chloro-1-(2,3-difluoro-4-methylphenyl)-2-(4,6-dimethylpyrimidin-2-yl)-5-fluoro- |
| | 2,3,4,9-tetrahydro-1H-pyrido[3,4-b]indole; |
| 206 | 6-chloro-1-(2,3-difluoro-4-methylphenyl)-5-fluoro-2-[4-methyl-6- |
| | (trifluoromethyl)pyrimidin-2-yl]-2,3,4,9-tetrahydro-1H-pyrido[3,4-b]indole; |
| 207 | 6-chloro-1-(4-methylphenyl)-2-[4-(trifluoromethyl)pyrimidin-2-yl]-2,3,4,9-tetrahydro- |
| | 1H-pyrido[3,4-b]indol-8-ol; |
| 208 | 5-{6-chloro-7-fluoro-2-[4-(trifluoromethyl)pyrimidin-2-yl]-2,3,4,9-tetrahydro-1H- |
| | pyrido[3,4-b]indol-1-yl}-6-fluoro-N,N-dimethylpyridin-2-amine; |
| 209 | 2-[4,6-bis(trifluoromethyl)pyrimidin-2-yl]-6-chloro-1-(2,3-difluoro-4-methylphenyl)- |
| | 5-fluoro-2,3,4,9-tetrahydro-1H-pyrido[3,4-b]indole; |
| 210 | 5-{6-chloro-5-fluoro-2-[4-(trifluoromethyl)pyrimidin-2-yl]-2,3,4,9-tetrahydro-1H- |
| | pyrido[3,4-b]indol-1-yl}-6-fluoro-N,N-dimethylpyridin-2-amine; |
| 211 | 6-chloro-5-fluoro-2-(5-fluoropyrimidin-2-yl)-1-[2-(morpholin-4-yl)pyrimidin-5-yl]- |
| | 2,3,4,9-tetrahydro-1H-pyrido[3,4-b]indole; |
| 212 | 5-[6-chloro-5-fluoro-2-(5-fluoropyrimidin-2-yl)-2,3,4,9-tetrahydro-1H-pyrido[3,4- |
| | b]indol-1-yl]pyrimidin-2-amine; |
| 213 | 6-chloro-1-(2,3-difluoro-4-methylphenyl)-2-[4-methyl-6-(trifluoromethyl)pyrimidin- |
| | 2-yl]-2,3,4,9-tetrahydro-1H-pyrido[3,4-b]indole; |
| 214 | 2-[6-chloro-1-(2,3-difluoro-4-methylphenyl)-5-fluoro-1,3,4,9-tetrahydro-2H- |
| | pyrido[3,4-b]indol-2-yl]-5-fluoropyrimidine-4-carbonitrile; |
| 215 | 2-[6-chloro-7-fluoro-1-(4-methylphenyl)-1,3,4,9-tetrahydro-2H-pyrido[3,4-b]indol-2- |
| | yl]pyrimidine-4,6-dicarbonitrile; |
| 216 | 2-[6-chloro-1-(2,3-difluoro-4-methylphenyl)-7-fluoro-1,3,4,9-tetrahydro-2H- |
| | pyrido[3,4-b]indol-2-yl]pyrimidine-4,6-dicarbonitrile; |
| 217 | 2-[6-chloro-5-fluoro-1-(4-methylphenyl)-1,3,4,9-tetrahydro-2H-pyrido[3,4-b]indol-2- |
| | yl]-5-fluoropyrimidine-4-carbonitrile; |
| 218 | 2-chloro-5-{6-chloro-2-[4-(trifluoromethyl)pyrimidin-2-yl]-2,3,4,9-tetrahydro-1H- |
| | pyrido[3,4-b]indol-1-yl}phenol; |
| 219 | 2-chloro-5-[6-chloro-2-(5-fluoropyrimidin-2-yl)-2,3,4,9-tetrahydro-1H-pyrido[3,4- |
| | b]indol-1-yl]phenol; |
| 220 | 6-chloro-1-(2,3-difluoro-4-methylphenyl)-2-[4-(trifluoromethyl)pyrimidin-2-yl]- |
| | 2,3,4,9-tetrahydro-1H-pyrido[3,4-b]indol-8-ol; |
| 221 | 5-chloro-2-[6-chloro-2-(5-fluoropyrimidin-2-yl)-2,3,4,9-tetrahydro-1H-pyrido[3,4- |
| | b]indol-1-yl]phenol; |
| 222 | 5-chloro-2-{6-chloro-2-[4-(trifluoromethyl)pyrimidin-2-yl]-2,3,4,9-tetrahydro-1H- |
| | pyrido[3,4-b]indol-1-yl}phenol; |
| 223 | 2-[(4-{6-chloro-7-fluoro-2-[4-(trifluoromethyl)pyrimidin-2-yl]-2,3,4,9-tetrahydro-1H- |
| | pyrido[3,4-b]indol-1-yl}phenyl)methoxy]ethan-1-ol; |
| 224 | 2-(6-chloro-7-fluoro-1-{4-[(1E)-4-hydroxybut-1-en-1-yl]phenyl}-1,3,4,9-tetrahydro- |
| | 2H-pyrido[3,4-b]indol-2-yl)pyrimidine-4-carbonitrile; |
| 225 | 5-{6-chloro-5-fluoro-2-[4-(trifluoromethyl)pyrimidin-2-yl]-2,3,4,9-tetrahydro-1H- |
| | pyrido[3,4-b]indol-1-yl}pyrimidin-2-amine; |
| 226 | 6-chloro-5-fluoro-1-(2-fluoropyridin-3-yl)-2-[4-(trifluoromethyl)pyrimidin-2-yl]- |
| | 2,3,4,9-tetrahydro-1H-pyrido[3,4-b]indole; |
| 227 | 2-[6-chloro-1-(2,3-difluorophenyl)-7-fluoro-1,3,4,9-tetrahydro-2H-pyrido[3,4-b]indol- |
| | 2-yl]pyrimidine-4,6-dicarbonitrile; |
| 228 | 2-[6-chloro-5-fluoro-1-(4-methylphenyl)-1,3,4,9-tetrahydro-2H-pyrido[3,4-b]indol-2- |
| | yl]pyrimidine-4,6-dicarbonitrile; |
| 229 | 2-[6-chloro-1-(2,3-difluoro-4-methylphenyl)-5-fluoro-1,3,4,9-tetrahydro-2H- |
| | pyrido[3,4-b]indol-2-yl]pyrimidine-4,6-dicarbonitrile; |
| 230 | 6-chloro-5-fluoro-1-[2-(1H-1,2,4-triazol-1-yl)pyrimidin-5-yl]-2-[4- |
| | (trifluoromethyl)pyrimidin-2-yl]-2,3,4,9-tetrahydro-1H-pyrido[3,4-b]indole; |
| 231 | 6-chloro-1-(4-methylphenyl)-2-[4-(trifluoromethyl)pyrimidin-2-yl]-2,3,4,9-tetrahydro- |
| | 1H-pyrido[3,4-b]indol-8-amine; |
| 232 | 2-[6-chloro-1-(2,3-difluoro-4-methylphenyl)-5-fluoro-1,3,4,9-tetrahydro-2H- |
| | pyrido[3,4-b]indol-2-yl]-6-methylpyrimidine-4-carbonitrile; |
| 233 | (1S)-6-chloro-1-(2,3-difluoro-4-methylphenyl)-2-[4-(trifluoromethyl)pyrimidin-2-yl]- |
| | 2,3,4,9-tetrahydro-1H-pyrido[3,4-b]indole; |
| 234 | (1S)-6-chloro-1-(2,3-difluoro-4-methylphenyl)-2-(4-methylpyrimidin-2-yl)-2,3,4,9- |
| | tetrahydro-1H-pyrido[3,4-b]indole; |
| 235 | 6-chloro-1-(2,3-difluoro-4-methylphenyl)-2-(5-fluoro-4-methylpyrimidin-2-yl)- |
| | 2,3,4,9-tetrahydro-1H-pyrido[3,4-b]indole; |
| 236 | 2-(6-chloro-5-fluoro-1-{4-[(1E)-4-hydroxybut-1-en-1-yl]phenyl}-1,3,4,9-tetrahydro- |
| | 2H-pyrido[3,4-b]indol-2-yl)pyrimidine-4-carbonitrile; |
| 237 | (3E)-4-{4-[6-chloro-5-fluoro-2-(4-methylpyrimidin-2-yl)-2,3,4,9-tetrahydro-1H- |
| | pyrido[3,4-b]indol-1-yl]phenyl}but-3-en-1-ol; |
| 238 | (1S)-6-chloro-1-(2,3-difluoro-4-methylphenyl)-2-(5-fluoropyrimidin-2-yl)-2,3,4,9- |
| | tetrahydro-1H-pyrido[3,4-b]indole; |
| 239 | (3E)-4-{4-[6-chloro-7-fluoro-2-(5-fluoro-4-methylpyrimidin-2-yl)-2,3,4,9-tetrahydro- |
| | 1H-pyrido[3,4-b]indol-1-yl]phenyl}but-3-en-1-ol; |
| 240 | 4-{4-[6-chloro-7-fluoro-2-(5-fluoro-4-methylpyrimidin-2-yl)-2,3,4,9-tetrahydro-1H- |
| | pyrido[3,4-b]indol-1-yl]phenyl}butan-1-ol; |
| 241 | (3E)-4-{4-[6-chloro-5-fluoro-2-(5-fluoro-4-methylpyrimidin-2-yl)-2,3,4,9-tetrahydro- |
| | 1H-pyrido[3,4-b]indol-1-yl]phenyl}but-3-en-1-ol; |
| 242 | (3E)-4-{4-[6-chloro-2-(4,6-dimethylpyrimidin-2-yl)-5-fluoro-2,3,4,9-tetrahydro-1H- |
| | pyrido[3,4-b]indol-1-yl]phenyl}but-3-en-1-ol; |
| 243 | (3E)-4-{4-[6-chloro-5-fluoro-2-(5-fluoro-4,6-dimethylpyrimidin-2-yl)-2,3,4,9- |
| | tetrahydro-1H-pyrido[3,4-b]indol-1-yl]phenyl}but-3-en-1-ol; |
| 244 | (3E)-4-{4-[6-chloro-2-(4,6-dimethylpyrimidin-2-yl)-7-fluoro-2,3,4,9-tetrahydro-1H- |
| | pyrido[3,4-b]indol-1-yl]phenyl}but-3-en-1-ol; |
| 245 | 4-{4-[6-chloro-2-(4,6-dimethylpyrimidin-2-yl)-7-fluoro-2,3,4,9-tetrahydro-1H- |
| | pyrido[3,4-b]indol-1-yl]phenyl}butan-1-ol; |
| 246 | 4-{4-[6-chloro-5-fluoro-2-(5-fluoro-4-methylpyrimidin-2-yl)-2,3,4,9-tetrahydro-1H- |
| | pyrido[3,4-b]indol-1-yl]phenyl}butan-1-ol; |
| 247 | 5-bromo-6-chloro-1-(4-chlorophenyl)-2-[4-(trifluoromethyl)pyrimidin-2-yl]-2,3,4,9- |
| | tetrahydro-1H-pyrido[3,4-b]indole; |
| 248 | 6-chloro-1-(2,3-difluoro-4-methylphenyl)-2-[4-(trifluoromethyl)pyrimidin-2-yl]- |
| | 2,3,4,9-tetrahydro-1H-pyrido[3,4-b]indol-8-amine; |
| 249 | 4-{4-[6-chloro-7-fluoro-2-(5-fluoro-4,6-dimethylpyrimidin-2-yl)-2,3,4,9-tetrahydro- |
| | 1H-pyrido[3,4-b]indol-1-yl]phenyl}butan-1-ol; |
| 250 | 6-chloro-1-(4-chlorophenyl)-2-[4-(trifluoromethyl)pyrimidin-2-yl]-2,3,4,9-tetrahydro- |
| | 1H-pyrido[3,4-b]indol-7-ol; |
| 251 | 6-chloro-1-[2-(morpholin-4-yl)pyrimidin-5-yl]-2-[4-(trifluoromethyl)pyrimidin-2-yl]- |
| | 2,3,4,9-tetrahydro-1H-pyrido[3,4-b]indole; |
| 252 | 2-{6-chloro-2-[4-(trifluoromethyl)pyrimidin-2-yl]-2,3,4,9-tetrahydro-1H-pyrido[3,4- |
| | bindol-1-yl}-6-fluorophenol; |
| 254 | 6-chloro-1-(2-fluoro-4-methylphenyl)-2-[4-(trifluoromethyl)pyrimidin-2-yl]-2,3,4,9- |
| | tetrahydro-1H-pyrido[3,4-b]indole; |
| 255 | 4-{6-chloro-2-[4-(trifluoromethyl)pyrimidin-2-yl]-2,3,4,9-tetrahydro-1H-pyrido[3,4- |
| | b]indol-1-yl}-3-fluorobenzonitrile; |
| 256 | 3-{6-chloro-2-[4-(trifluoromethyl)pyrimidin-2-yl]-2,3,4,9-tetrahydro-1H-pyrido[3,4- |
| | b]indol-1-yl}-2-fluorophenol; |
| 257 | 2-{6-chloro-2-[4-(trifluoromethyl)pyrimidin-2-yl]-2,3,4,9-tetrahydro-1H-pyrido[3,4- |
| | b]indol-1-yl}-5-methylphenol; |
| 258 | 5-{6-chloro-2-[4-(trifluoromethyl)pyrimidin-2-yl]-2,3,4,9-tetrahydro-1H-pyrido[3,4- |
| | b]indol-1-yl}-2-methylphenol; |
| 259 | 6-chloro-1-(2-fluoro-3-methylphenyl)-2-[4-(trifluoromethyl)pyrimidin-2-yl]-2,3,4,9- |
| | tetrahydro-1H-pyrido[3,4-b]indole; |
| 260 | 6-chloro-1-[4-(1H-imidazol-1-yl)phenyl]-2-[4-(trifluoromethyl)pyrimidin-2-yl]- |
| | 2,3,4,9-tetrahydro-1H-pyrido[3,4-b]indole; |
| 261 | 6-chloro-1-[4-(1H-pyrazol-1-yl)phenyl]-2-[4-(trifluoromethyl)pyrimidin-2-yl]-2,3,4,9- |
| | tetrahydro-1H-pyrido[3,4-b]indole; |
| 262 | 6-chloro-1-[4-(morpholin-4-yl)phenyl]-2-[4-(trifluoromethyl)pyrimidin-2-yl]-2,3,4,9- |
| | tetrahydro-1H-pyrido[3,4-b]indole; |
| 263 | 6-chloro-2-[4-(trifluoromethyl)pyrimidin-2-yl]-1-(2,3,4-trifluorophenyl)-2,3,4,9- |
| | tetrahydro-1H-pyrido[3,4-b]indole; |
| 264 | 6-bromo-1-(4-methylphenyl)-2-[4-(trifluoromethyl)pyrimidin-2-yl]-2,3,4,9- |
| | tetrahydro-1H-pyrido[3,4-b]indole; |
| 265 | 1-(4-methylphenyl)-6-nitro-2-[4-(trifluoromethyl)pyrimidin-2-yl]-2,3,4,9-tetrahydro- |
| | 1H-pyrido[3,4-b]indole; |
| 266 | 6-chloro-1-(2,3-difluoro-4-methylphenyl)-5-nitro-2-[4-(trifluoromethyl)pyrimidin-2- |
| | yl]-2,3,4,9-tetrahydro-1H-pyrido[3,4-b]indole; |
| 267 | 6-chloro-1-(4-chloro-2-fluorophenyl)-2-[4-(trifluoromethyl)pyrimidin-2-yl]-2,3,4,9- |
| | tetrahydro-1H-pyrido[3,4-b]indole; |
| 268 | 1-(4-bromo-2-fluorophenyl)-6-chloro-2-[4-(trifluoromethyl)pyrimidin-2-yl]-2,3,4,9- |
| | tetrahydro-1H-pyrido[3,4-b]indole; |
| 269 | 6-chloro-1-[4-(4H-1,2,4-triazol-4-yl)phenyl]-2-[4-(trifluoromethyl)pyrimidin-2-yl]- |
| | 2,3,4,9-tetrahydro-1H-pyrido[3,4-b]indole; |
| 270 | 1-(4-bromo-2,3-difluorophenyl)-6-chloro-2-[4-(trifluoromethyl)pyrimidin-2-yl]- |
| | 2,3,4,9-tetrahydro-1H-pyrido[3,4-b]indole; |
| 271 | 1-(2,3-difluoro-4-methylphenyl)-6-methyl-2-[4-(trifluoromethyl)pyrimidin-2-yl]- |
| | 2,3,4,9-tetrahydro-1H-pyrido[3,4-b]indole; |
| 272 | 1-(2,3-difluoro-4-methylpheny])-6-methyl-2-[4-(trifluoromethyl)pyrimidin-2-yl]- |
| | 2,3,4,9-tetrahydro-1H-pyrido[3,4-b]indol-8-amine; |
| 273 | 6-chloro-1-(2,3-difluoro-4-methylphenyl)-2-[4-(trifluoromethyl)pyrimidin-2-yl]- |
| | 2,3,4,9-tetrahydro-1H-pyrido[3,4-b]indol-5-amine; |
| 274 | 6-chloro-1-[2-fluoro-4-(1H-imidazol-1-yl)phenyl]-2-(pyrimidin-2-yl)-2,3,4,9- |
| | tetrahydro-1H-pyrido[3,4-b]indole; |
| 275 | 6-chloro-1-[2-fluoro-4-(1H-imidazol-1-yl)phenyl]-2-[4-(trifluoromethyl)pyrimidin-2- |
| | yl]-2,3,4,9-tetrahydro-1H-pyrido[3,4-b]indole; |
| 276 | 1-(2,3-difluoro-4-methylphenyl)-6-methyl-2-[4-(trifluoromethyl)pyrimidin-2-yl]- |
| | 2,3,4,9-tetrahydro-1H-pyrido[3,4-b]indol-8-ol; |
| 277 | 6-chloro-1-[2-fluoro-4-(morpholin-4-yl)phenyl]-2-[4-(trifluoromethyl)pyrimidin-2- |
| | yl]-2,3,4,9-tetrahydro-1H-pyrido[3,4-b]indole; |
| 278 | 4-{6-chloro-2-[4-(trifluoromethyl)pyrimidin-2-yl]-2,3,4,9-tetrahydro-1H-pyrido[3,4- |
| | b]indol-1-yl}-2,3-difluorobenzonitrile; |
| 279 | 6-chloro-1-[2,3-difluoro-4-(morpholin-4-yl)phenyl]-2-[4-(trifluoromethyl)pyrimidin- |
| | 2-yl]-2,3,4,9-tetrahydro-1H-pyrido[3,4-b]indolc; |
| 280 | butyl 2-[6-chloro-1-(4-methylphenyl)-1,3,4,9-tetrahydro-2H-pyrido[3,4-b]indol-2- |
| | yl]pyrimidine-4-carboxylate; |
| 281 | butyl 2-[6-chloro-1-(2,3-difluoro-4-methylphenyl)-1,3,4,9-tetrahydro-2H-pyrido[3,4- |
| | b]indol-2-yl]pyrimidine-4-carboxylate; |
| 282 | 6-chloro-1-(2,3-difluoro-4-methylphenyl)-2-[4-(trifluoromethyl)pyrimidin-2-yl]- |
| | 2,3,4,9-tetrahydro-1H-pyrido[3,4-b]indol-7-amine; |
| 285 | 6-chloro-1-[2,3-difluoro-4-(1H-imidazol-1-yl)phenyl]-2-[4- |
| | (trifluoromethyl)pyrimidin-2-yl]-2,3,4,9-tetrahydro-1H-pyrido[3,4-b]indole; |
| 286 | 6-chloro-2-(4,6-dimethylpyrimidin-2-yl)-1-(4-methylphenyl)-2,3,4,9-tetrahydro-1H- |
| | pyrido[3,4-b]indole; |
| 287 | 1-(4-methylphenyl)-6-(2H-1,2,3-triazol-2-yl)-2-[4-(trifluoromethyl)pyrimidin-2-yl]- |
| | 2,3,4,9-tetrahydro-1H-pyrido[3,4-b]indole; |
| 288 | 1-[2-(morpholin-4-yl)pyrimidin-5-yl]-6-(2H-1,2,3-triazol-2-yl)-2-[4- |
| | (trifluoromethyl)pyrimidin-2-yl]-2,3,4,9-tetrahydro-1H-pyrido[3,4-b]indole; |
| 291 | 6-ethenyl-1-(4-methylphenyl)-2-[4-(trifluoromethyl)pyrimidin-2-yl]-2,3,4,9- |
| | tetrahydro-1H-pyrido[3,4-b]indole; |
| 298 | 6-bromo-1-[2-(morpholin-4-yl)pyrimidin-5-yl]-2-[4-(trifluoromethyl)pyrimidin-2-yl]- |
| | 2,3,4,9-tetrahydro-1H-pyrido[3,4-b]indole; |
| 301 | 1-[2-(morpholin-4-yl)pyrimidin-5-yl]-6-(1H-pyrazol-1-yl)-2-[4- |
| | (trifluoromethyl)pyrimidin-2-yl]-2,3,4,9-tetrahydro-1H-pyrido[3,4-b]indole; |
| 302 | 6-methoxy-1-[2-(morpholin-4-yl)pyrimidin-5-yl]-2-[4-(trifluoromethyl)pyrimidin-2- |
| | yl]-2,3,4,9-tetrahydro-1H-pyrido[3,4-b]indole; |
| 303 | 6-chloro-1-[6-(morpholin-4-yl)pyridin-3-yl]-2-[4-(trifluoromethyl)pyrimidin-2-yl]- |
| | 2,3,4,9-tetrahydro-1H-pyrido[3,4-b]indole; |
| 305 | 1-(2,3-difluoro-4-methylphenyl)-6-nitro-2-[4-(trifluoromethyl)pyrimidin-2-yl]- |
| | 2,3,4,9-tetrahydro-1H-pyrido[3,4-b]indole; |
| 306 | 2-[6-chloro-1-(2,3-difluoro-4-methylphenyl)-1,3,4,9-tetrahydro-2H-pyrido[3,4- |
| | b]indol-2-yl]-N,N-dimethylpyrimidin-4-amine; |
| 307 | 2-[6-chloro-1-(4-methylphenyl)-1,3,4,9-tetrahydro-2H-pyrido[3,4-b]indol-2-yl]-N,N- |
| | dimethylpyrimidin-4-amine; |
| 308 | 6-chloro-2-(4,6-dimethylpyrimidin-2-yl)-1-[2-(morpholin-4-yl)pyrimidin-5-yl]- |
| | 2,3,4,9-tetrahydro-1H-pyrido[3,4-b]indole; |
| 310 | 6-chloro-1-[2-(morpholin-4-yl)pyrimidin-5-yl]-2-[4-(trifluoromethyl)pyrimidin-2-yl]- |
| | 2,3,4,9-tetrahydro-1H-pyrido[3,4-b]indol-8-amine; |
| 311 | 6-chloro-1-[2-(morpholin-4-yl)pyrimidin-5-yl]-2-[4-(trifluoromethyl)pyrimidin-2-yl]- |
| | 2,3,4,9-tetrahydro-1H-pyrido[3,4-b]indol-7-amine; |
| 312 | 6-chloro-2-(4-methylpyrimidin-2-yl)-1-[2-(morpholin-4-yl)pyrimidin-5-yl]-2,3,4,9- |
| | tetrahydro-1H-pyrido[3,4-b]indole; |
| 314 | 1-[2-(morpholin-4-yl)pyrimidin-5-yl]-6-(1H-pyrrol-1-yl)-2-[4- |
| | (trifluoromethyl)pyrimidin-2-yl]-2,3,4,9-tetrahydro-1H-pyrido[3,4-b]indole; |
| 316 | 1-[6-(morpholin-4-yl)pyridin-3-yl]-6-(2H-1,2,3-triazol-2-yl)-2-[4- |
| | (trifluoromethyl)pyrimidin-2-yl]-2,3,4,9-tetrahydro-1H-pyrido[3,4-b]indole; |
| 319 | 6-chloro-1-(2,6-dichloropyridin-3-yl)-2-[4-(trifluoromethyl)pyrimidin-2-yl]-2,3,4,9- |
| | tetrahydro-1H-pyrido[3,4-b]indole; |
| 323 | 1-[2-(morpholin-4-yl)pyrimidin-5-yl]-6-nitro-2-[4-(trifluoromethyl)pyrimidin-2-yl]- |
| | 2,3,4,9-tetrahydro-1H-pyrido[3,4-b]indole; |
| 326 | 6-chloro-1-[2-(morpholin-4-yl)pyrimidin-5-yl]-5-nitro-2-[4- |
| | (trifluoromethyl)pyrimidin-2-yl]-2,3,4,9-tetrahydro-1H-pyrido[3,4-b]indole; |
| 328 | 6-chloro-1-[2-(morpholin-4-yl)pyrimidin-5-yl]-2-[4-(trifluoromethyl)pyrimidin-2-yl]- |
| | 2,3,4,9-tetrahydro-1H-pyrido[3,4-b]indol-5-amine; |
| 329 | N,N-dimethyl-1-[2-(morpholin-4-yl)pyrimidin-5-yl]-2-[4-(trifluoromethyl)pyrimidin- |
| | 2-yl]-2,3,4,9-tetrahydro-1H-pyrido[3,4-b]indol-6-amine; |
| 330 | 1-(2,3-difluoro-4-methylphenyl)-N,N-dimethyl-2-[4-(trifluoromethyl)pyrimidin-2-yl]- |
| | 2,3,4,9-tetrahydro-1H-pyrido[3,4-b]indol-6-amine; |
| 332 | 6-chloro-1-(4-methylphenyl)-2-(4-methylpyrimidin-2-yl)-2,3,4,9-tetrahydro-1H- |
| | pyrido[3,4-b]indole; |
| 334 | 6-chloro-2-[4-(difluoromethyl)pyrimidin-2-yl]-1-[2-(morpholin-4-yl)pyrimidin-5-yl]- |
| | 2,3,4,9-tetrahydro-1H-pyrido[3,4-b]indole; |
| 335 | 6-chloro-1-[2-(2-oxa-5-azabicyclo[2.2.1]heptan-5-yl)pyrimidin-5-yl]-2-[4- |
| | (trifluoromethyl)pyrimidin-2-yl]-2,3,4,9-tetrahydro-1H-pyrido[3,4-b]indole; |
| 337 | 6-chloro-1-[2-(3-oxa-8-azabicyclo[3.2.1]octan-8-yl)pyrimidin-5-yl]-2-[4- |
| | (trifluoromethyl)pyrimidin-2-yl]-2,3,4,9-tetrahydro-1H-pyrido[3,4-b]indole; |
| 340 | 2-[4,6-bis(trifluoromethyl)pyrimidin-2-yl]-6-chloro-1-(2,3-difluoro-4-methylphenyl)- |
| | 2,3,4,9-tetrahydro-1H-pyrido[3,4-b]indole; |
| 341 | 6-chloro-1-(2-fluorophenyl)-2-[4-(trifluoromethyl)pyrimidin-2-yl]-2,3,4,9-tetrahydro- |
| | 1H-pyrido[3,4-b]indole; |
| 342 | 6-chloro-1-(2,3-difluoro-4-methylphenyl)-2-(4,6-dimethylpyrimidin-2-yl)-2,3,4,9- |
| | tetrahydro-1H-pyrido[3,4-b]indole; |
| 343 | 6-chloro-1-(4-methylphenyl)-2-[4-(trifluoromethyl)pyrimidin-2-yl]-2,3,4,9-tetrahydro- |
| | 1H-pyrido[3,4-b]indole; |
| 344 | 1-[2-(azetidin-1-yl)pyrimidin-5-yl]-6-chloro-2-[4-(trifluoromethyl)pyrimidin-2-yl]- |
| | 2,3,4,9-tetrahydro-1H-pyrido[3,4-b]indole; |
| 349 | 6-chloro-1-(4-chlorophenyl)-2-[4-(trifluoromethyl)pyrimidin-2-yl]-2,3,4,9-tetrahydro- |
| | 1H-pyrido[3,4-b]indole; |
| 350 | 6-chloro-2-(4,6-dimethylpyrimidin-2-yl)-1-[6-(morpholin-4-yl)pyridin-3-yl]-2,3,4,9- |
| | tetrahydro-1H-pyrido[3,4-b]indole; |
| 351 | 6-chloro-2-(4-methylpyrimidin-2-yl)-1-[6-(morpholin-4-yl)pyridin-3-yl]-2,3,4,9- |
| | tetrahydro-1H-pyrido[3,4-b]indol-8-amine; |
| 352 | 6-chloro-2-(4-methylpyrimidin-2-yl)-1-[6-(morpholin-4-yl)pyridin-3-yl]-2,3,4,9- |
| | tetrahydro-1H-pyrido[3,4-b]indole; |
| 353 | 2-{6-chloro-2-[4-(trifluoromethyl)pyrimidin-2-yl]-2,3,4,9-tetrahydro-1H-pyrido[3,4- |
| | b]indol-1-yl}phenol; |
| 354 | 6-chloro-1-(3-fluoro-4-methylphenyl)-2-[4-(trifluoromethyl)pyrimidin-2-yl]-2,3,4,9- |
| | tetrahydro-1H-pyrido[3,4-b]indole; |
| 356 | 2-{6-chloro-1-[6-(morpholin-4-yl)pyridin-3-yl]-1,3,4,9-tetrahydro-2H-pyrido[3,4- |
| | b]indol-2-yl}pyrimidine-4-carbonitrile; |
| ¹357 | 2-[4,6-bis(trifluoromethyl)pyrimidin-2-yl]-6-chloro-1-[6-(morpholin-4-yl)pyridin-3- |
| | yl]-2,3,4,9-tetrahydro-1H-pyrido[3,4-b]indole; |
| ¹358 | 2-[4,6-bis(trifluoromethyl)pyrimidin-2-yl]-6-chloro-1-[2-(morpholin-4-yl)pyrimidin- |
| | 5-yl]-2,3,4,9-tetrahydro-1H-pyrido[3,4-b]indole; |
| 359 | 2-{6-chloro-1-[2-(morpholin-4-yl)pyrimidin-5-yl]-1,3,4,9-tetrahydro-2H-pyrido[3,4- |
| | b]indol-2-yl}pyrimidine-4-carbonitrile; |
| 360 | 2-[4,6-bis(trifluoromethyl)pyrimidin-2-yl]-6-chloro-1-(4-methylphenyl)-2,3,4,9- |
| | tetrahydro-1H-pyrido[3,4-b]indole; |
| 361 | 6-chloro-1-(4-methylphenyl)-2-[4-methyl-6-(trifluoromethyl)pyrimidin-2-yl]-2,3,4,9- |
| | tetrahydro-1H-pyrido[3,4-b]indole; |
| 362 | 2-{6-chloro-1-[2-(morpholin-4-yl)pyrimidin-5-yl]-1,3,4,9-tetrahydro-2H-pyrido[3,4- |
| | b]indol-2-yl}-6-methylpyrimidine-4-carbonitrile; |
| 363 | 2-[6-chloro-1-(2,3-difluoro-4-methylphenyl)-1,3,4,9-tetrahydro-2H-pyrido[3,4- |
| | b]indol-2-yl]pyrimidine-4-carbonitrile; |
| 364 | 6-chloro-2-(5-fluoropyrimidin-2-yl)-1-[2-(morpholin-4-yl)pyrimidin-5-yl]-2,3,4,9- |
| | tetrahydro-1H-pyrido[3,4-b]indole; |
| 365 | 6-chloro-2-(5-fluoro-4-methylpyrimidin-2-yl)-1-[6-(morpholin-4-yl)pyridin-3-yl]- |
| | 2,3,4,9-tetrahydro-1H-pyrido[3,4-b]indole; |
| 366 | 6-chloro-2-(5-fluoro-4-methylpyrimidin-2-yl)-1-[2-(morpholin-4-yl)pyrimidin-5-yl]- |
| | 2,3,4,9-tetrahydro-1H-pyrido[3,4-b]indole; |
| 367 | 6-chloro-2-(5-fluoro-4,6-dimethylpyrimidin-2-yl)-1-[6-(morpholin-4-yl)pyridin-3-yl]- |
| | 2,3,4,9-tetrahydro-1H-pyrido[3,4-b]indole; |
| 368 | 6-chloro-2-(5-fluoro-4,6-dimethylpyrimidin-2-yl)-1-[2-(morpholin-4-yl)pyrimidin-5- |
| | yl]-2,3,4,9-tetrahydro-1H-pyrido[3,4-b]indole; |
| 369 | 6-chloro-1-[2-(4-methylpiperazin-1-yl)pyrimidin-5-yl]-2-[4- |
| | (trifluoromethyl)pyrimidin-2-yl]-2,3,4,9-tetrahydro-1H-pyrido[3,4-b]indole; |
| 371 | 6-chloro-1-(3-fluorophenyl)-2-[4-(trifluoromethyl)pyrimidin-2-yl]-2,3,4,9-tetrahydro- |
| | 1H-pyrido[3,4-b]indole; |
| 372 | 6-chloro-2-[4-methyl-6-(trifluoromethyl)pyrimidin-2-yl]-1-[6-(morpholin-4- |
| | yl)pyridin-3-yl]-2,3,4,9-tetrahydro-1H-pyrido[3,4-b]indole; |
| 373 | 6-chloro-1-{6-[(1R,4R)-2-oxa-5-azabicyclo[2.2.1]heptan-5-yl]pyridin-3-yl}-2-[4- |
| | (trifluoromethyl)pyrimidin-2-yl]-2,3,4,9-tetrahydro-1H-pyrido[3,4-b]indole; |
| 374 | 6-chloro-2-[4-methyl-6-(trifluoromethyl)pyrimidin-2-yl]-1-[2-(morpholin-4- |
| | yl)pyrimidin-5-yl]-2,3,4,9-tetrahydro-1H-pyrido[3,4-b]indole; |
| 375 | 6-chloro-1-{6-[(1S,4S)-2-oxa-5-azabicyclo[2.2.1]heptan-5-yl]pyridin-3-yl}-2-[4- |
| | (trifluoromethyl)pyrimidin-2-yl]-2,3,4,9-tetrahydro-1H-pyrido[3,4-b]indole; |
| 378 | 2-[6-chloro-1-(4-methylphenyl)-1,3,4,9-tetrahydro-2H-pyrido[3,4-b]indol-2- |
| | yl]pyrimidine-4-carbonitrile; |
| 379 | 2-[6-chloro-1-(4-methylphenyl)-1,3,4,9-tetrahydro-2H-pyrido[3,4-b]indol-2-yl]-6- |
| | methylpyrimidine-4-carbonitrile; |
| 381 | 2-[4,6-bis(trifluoromethyl)pyrimidin-2-yl]-6-chloro-1-[2-(4-methylpiperazin-1- |
| | yl)pyrimidin-5-yl]-2,3,4,9-tetrahydro-1H-pyrido[3,4-b]indole; |
| 382 | 6-chloro-2-(5-fluoropyrimidin-2-yl)-1-[6-(morpholin-4-yl)pyridin-3-yl]-2,3,4,9- |
| | tetrahydro-1H-pyrido[3,4-b]indole; |
| 383 | 6-chloro-1-[2-(morpholin-4-yl)pyrimidin-5-yl]-2-[5-(trifluoromethyl)-1,2,4-triazin-3- |
| | yl]-2,3,4,9-tetrahydro-1H-pyrido[3,4-b]indole; |
| 384 | 6-chloro-2-(5-methyl-1,2,4-triazin-3-yl)-1-[2-(morpholin-4-yl)pyrimidin-5-yl]-2,3,4,9- |
| | tetrahydro-1H-pyrido[3,4-b]indole; |
| 385 | 2-[(1S)-6-chloro-1-(2,3-difluoro-4-methylphenyl)-1,3,4,9-tetrahydro-2H-pyrido[3,4- |
| | b]indol-2-yl]-5-fluoropyrimidine-4-carbonitrile; |
| 391 | 6-chloro-1-[2-(2-oxa-6-azaspiro[3.3]heptan-6-yl)pyrimidin-5-yl]-2-[4- |
| | (trifluoromethyl)pyrimidin-2-yl]-2,3,4,9-tetrahydro-1H-pyrido[3,4-b]indole; |
| 392 | 2-[(1S)-6-chloro-1-(4-methylphenyl)-1,3,4,9-tetrahydro-2H-pyrido[3,4-b]indol-2-yl]- |
| | 5-fluoropyrimidine-4-carbonitrile; |
| 393 | (1S)-6-chloro-2-(5-fluoropyrimidin-2-yl)-1-(4-methylphenyl)-2,3,4,9-tetrahydro-1H- |
| | pyrido[3,4-b]indole; |
| 394 | (1S)-6-chloro-2-(5-fluoro-4-methylpyrimidin-2-yl)-1-(4-methylphenyl)-2,3,4,9- |
| | tetrahydro-1H-pyrido[3,4-b]indole; |
| 395 | (1S)-6-chloro-2-(5-fluoro-4,6-dimethylpyrimidin-2-yl)-1-(4-methylphenyl)-2,3,4,9- |
| | tetrahydro-1H-pyrido[3,4-b]indole; |
| 396 | 2-{(1S)-6-chloro-1-[2-(morpholin-4-yl)pyrimidin-5-yl]-1,3,4,9-tetrahydro-2H- |
| | pyrido[3,4-b]indol-2-yl}-5-fluoropyrimidine-4-carbonitrile; |
| 397 | 2-[(1S)-6-chloro-1-(4-methylphenyl)-1,3,4,9-tetrahydro-2H-pyrido[3,4-b]indol-2- |
| | yl]pyrimidine-4,6-dicarbonitrile; |
| 398 | 2-{(1S)-6-chloro-1-[2-(morpholin-4-yl)pyrimidin-5-yl]-1,3,4,9-tetrahydro-2H- |
| | pyrido[3,4-b]indol-2-yl}pyrimidine-4,6-dicarbonitrile; |
| 400 | 2-{(1S)-6-chloro-1-[6-(morpholin-4-yl)pyridin-3-yl]-1,3,4,9-tetrahydro-2H- |
| | pyrido[3,4-b]indol-2-yl}-5-fluoropyrimidine-4-carbonitrile; |
| 401 | 6-chloro-1-(2,3-difluoro-4-methylphenyl)-2-(5-fluoropyrimidin-2-yl)-2,3,4,9- |
| | tetrahydro-1H-pyrido[3,4-b]indole; |
| 403 | 6-chloro-1-(2,3-difluoro-4-methylphenyl)-2-[5-(trifluoromethyl)-1,2,4-triazin-3-yl]- |
| | 2,3,4,9-tetrahydro-1H-pyrido[3,4-b]indole; |
| 407 | (1S)-2-[4,6-bis(trifluoromethyl)pyrimidin-2-yl]-6-chloro-1-(2,3-difluoro-4- |
| | methylphenyl)-2,3,4,9-tetrahydro-1H-pyrido[3,4-b]indole; |
| 408 | (1S)-6-chloro-1-[2-(morpholin-4-yl)pyrimidin-5-yl]-2-[4-(trifluoromethyl)pyrimidin- |
| | 2-yl]-2,3,4,9-tetrahydro-1H-pyrido[3,4-b]indole; |
| 409 | (1S)-2-[4,6-bis(trifluoromethyl)pyrimidin-2-yl]-6-chloro-1-[6-(morpholin-4- |
| | yl)pyridin-3-yl]-2,3,4,9-tetrahydro-1H-pyrido[3,4-b]indole; |
| 410 | (1S)-2-[4,6-bis(trifluoromethyl)pyrimidin-2-yl]-6-chloro-1-[2-(morpholin-4- |
| | yl)pyrimidin-5-yl]-2,3,4,9-tetrahydro-1H-pyrido[3,4-b]indole; |
| 412 | (1S)-6-chloro-1-(2,3-difluoro-4-methylphenyl)-2-(5-fluoro-4,6-dimethylpyrimidin-2- |
| | yl)-2,3,4,9-tetrahydro-1H-pyrido[3,4-b]indole; |
| 413 | 2-[(1S)-6-chloro-1-(2,3-difluoro-4-methylphenyl)-1,3,4,9-tetrahydro-2H-pyrido[3,4- |
| | b]indol-2-yl]-5-fluoro-6-methylpyrimidine-4-carbonitrile; |
| 416 | 2-[(1S)-6-chloro-1-(2,3-difluoro-4-methylphenyl)-1,3,4,9-tetrahydro-2H-pyrido[3,4- |
| | b]indol-2-yl]-6-(trifluoromethyl)pyrimidine-4-carbonitrile; |
| 417 | 2-[(1S)-6-chloro-1-(4-methylphenyl)-1,3,4,9-tetrahydro-2H-pyrido[3,4-b]indol-2-yl]- |
| | 5-fluoro-6-methylpyrimidine-4-carbonitrile; |
| 419 | (1S)-2-[4,6-bis(trifluoromethyl)pyrimidin-2-yl]-6-chloro-1-[2-(4-methylpiperazin-1- |
| | yl)pyrimidin-5-yl]-2,3,4,9-tetrahydro-1H-pyrido[3,4-b]indole; |
| 421 | 2-{(1S)-6-chloro-1-[2-(morpholin-4-yl)pyrimidin-5-yl]-1,3,4,9-tetrahydro-2H- |
| | pyrido[3,4-b]indol-2-yl}-5-fluoro-6-methylpyrimidine-4-carbonitrile; |
| 422 | 6-chloro-1-(4-fluorophenyl)-2-[4-(trifluoromethyl)pyrimidin-2-yl]-2,3,4,9-tetrahydro- |
| | 1H-pyrido[3,4-b]indole; |
| 423 | 6-chloro-1-(2,4-difluorophenyl)-2-[4-(trifluoromethyl)pyrimidin-2-yl]-2,3,4,9- |
| | tetrahydro-1H-pyrido[3,4-b]indole; |
| 424 | 2-{(1S)-6-chloro-1-[6-(morpholin-4-yl)pyridin-3-yl]-1,3,4,9-tetrahydro-2H- |
| | pyrido[3,4-b]indol-2-yl}-5-fluoro-6-methylpyrimidine-4-carbonitrile; |
| 425 | 2-[(1S)-6-chloro-1-(4-methylphenyl)-1,3,4,9-tetrahydro-2H-pyrido[3,4-b]indol-2-yl]- |
| | 6-(trifluoromethyl)pyrimidine-4-carbonitrile; |
| 429 | 6-chloro-1-[6-(morpholin-4-yl)pyridin-3-yl]-2-[5-(trifluoromethyl)-1,2,4-triazin-3-yl]- |
| | 2,3,4,9-tetrahydro-1H-pyrido[3,4-b]indole; |
| 430 | 6-chloro-1-(4-methylphenyl)-2-[5-(trifluoromethyl)-1,2,4-triazin-3-yl]-2,3,4,9- |
| | tetrahydro-1H-pyrido[3,4-b]indole; |
| 431 | 6-chloro-1-(2,3-difluoro-4-methylphenyl)-2-[6-(trifluoromethyl)-1,2,4-triazin-3-yl]- |
| | 2,3,4,9-tetrahydro-1H-pyrido[3,4-b]indole; |
| 432 | 6-chloro-1-[2-fluoro-6-(morpholin-4-yl)pyridin-3-yl]-2-[4-(trifluoromethyl)pyrimidin- |
| | 2-yl]-2,3,4,9-tetrahydro-1H-pyrido[3,4-b]indole; |
| 433 | 2-{(1S)-6-chloro-1-[6-(morpholin-4-yl)pyridin-3-yl]-1,3,4,9-tetrahydro-2H- |
| | pyrido[3,4-b]indol-2-yl}-6-(trifluoromethyl)pyrimidine-4-carbonitrile; |
| 434 | 2-{(1S)-6-chloro-1-[2-(morpholin-4-yl)pyrimidin-5-yl]-1,3,4,9-tetrahydro-2H- |
| | pyrido[3,4-b]indol-2-yl}-6-(trifluoromethyl)pyrimidine-4-carbonitrile; |
| 487 | (1S)-2-[4,6-bis(trifluoromethyl)pyrimidin-2-yl]-6-bromo-1-[6-(morpholin-4- |
| | yl)pyridin-3-yl]-2,3,4,9-tetrahydro-1H-pyrido[3,4-b]indole; |
| 490 | 2-[4,6-bis(trifluoromethyl)pyrimidin-2-yl]-1-(2,3-difluoro-4-methylphenyl)-6-fluoro- |
| | 2,3,4,9-tetrahydro-1H-pyrido[3,4-b]indole; |
| 493 | 1-(2,3-difluoro-4-methylphenyl)-6-fluoro-2-[4-(trifluoromethyl)pyrimidin-2-yl]- |
| | 2,3,4,9-tetrahydro-1H-pyrido[3,4-b]indole; |
| 496 | (1S)-2-[4,6-bis(trifluoromethyl)pyrimidin-2-yl]-6-chloro-1-[2-(3,3-dimethylpiperazin- |
| | 1-yl)pyrimidin-5-yl]-2,3,4,9-tetrahydro-1H-pyrido[3,4-b]indole; |
| 499 | (1S)-2-[4,6-bis(trifluoromethyl)pyrimidin-2-yl]-6-bromo-1-[2-(4-methylpiperazin-1- |
| | yl)pyrimidin-5-yl]-2,3,4,9-tetrahydro-1H-pyrido\|3,4-b]indole; |
| 500 | (1S)-2-[4,6-bis(trifluoromethyl)pyrimidin-2-yl]-1-[2-(4-methylpiperazin-1- |
| | yl)pyrimidin-5-yl]-6-(2H-1,2,3-triazol-2-yl)-2,3,4,9-tetrahydro-1H-pyrido[3,4- |
| | b]indole; |
| 502 | 1-(2,3-difluoro-4-methylphenyl)-6-(2H-1,2,3-triazol-2-yl)-2-[4- |
| | (trifluoromethyl)pyrimidin-2-yl]-2,3,4,9-tetrahydro-1H-pyrido[3,4-b]indole; |
| 503 | (1S)-2-[4,6-bis(trifluoromethyl)pyrimidin-2-yl]-6-chloro-1-{2-[(3R,5S)-3,5- |
| | dimethylpiperazin-1-yl]pyrimidin-5-yl}-2,3,4,9-tetrahydro-1H-pyrido[3,4-b]indole; |
| 511 | (1S)-2-[4,6-bis(trifluoromethyl)pyrimidin-2-yl]-6-chloro-1-{2-[(3R)-3- |
| | methylpiperazin-1-yl]pyrimidin-5-yl}-2,3,4,9-tetrahydro-1H-pyrido[3,4-b]indole; |
| 512 | (1S)-2-[4,6-bis(trifluoromethyl)pyrimidin-2-yl]-6-chloro-1-[2-(4,7- |
| | diazaspiro[2.5]octan-7-yl)pyrimidin-5-yl]-2,3,4,9-tetrahydro-1H-pyrido[3,4-b]indole; |
| 513 | 1-[6-(4-methylpiperazin-1-yl)pyridin-3-yl]-6-(2H-1,2,3-triazol-2-yl)-2-[4- |
| | (trifluoromethyl)pyrimidin-2-yl]-2,3,4,9-tetrahydro-1H-pyrido[3,4-b]indole; |
| 514 | (1S)-2-[4,6-bis(trifluoromethyl)pyrimidin-2-yl]-1-[2-(morpholin-4-yl)pyrimidin-5-yl]- |
| | 6-(2H-1,2,3-triazol-2-yl)-2,3,4,9-tetrahydro-1H-pyrido[3,4-b]indole; |
| 517 | (1S)-2-[4,6-bis(trifluoromethyl)pyrimidin-2-yl]-6-chloro-1-{2-[(3S)-3- |
| | methylpiperazin-1-yl]pyrimidin-5-yl}-2,3,4,9-tetrahydro-1H-pyrido[3,4-b]indole; |
| 518 | (1S)-2-[4,6-bis(trifluoromethyl)pyrimidin-2-yl]-6-bromo-1-(2,3-difluoro-4- |
| | methylphenyl)-2,3,4,9-tetrahydro-1H-pyrido[3,4-b]indole; |
| 519 | (1S)-2-[4,6-bis(trifluoromethyl)pyrimidin-2-yl]-1-(2,3-difluoro-4-methylphenyl)-6- |
| | (2H-1,2,3-triazol-2-yl)-2,3,4,9-tetrahydro-1H-pyrido[3,4-b]indole; |
| 520 | (1S)-2-[4,6-bis(trifluoromethyl)pyrimidin-2-yl]-1-(2,3-difluoro-4-methylphenyl)-6-(4- |
| | methyl-2H-1,2,3-triazol-2-yl)-2,3,4,9-tetrahydro-1H-pyrido[3,4-b]indole; and, |
| 528 | (1S)-2-[4,6-bis(trifluoromethyl)pyrimidin-2-yl]-1-(2,3-difluoro-4-methylphenyl)-6- |
| | methoxy-2,3,4,9-tetrahydro-1H-pyrido[3,4-b]indole; |

wherein the form of the compound is selected from the group consisting of a salt, hydrate, solvate, racemate, enantiomer, diastereomer, stereoisomer, and tautomer form thereof.

Another aspect of the compound of Formula (I) or a form thereof includes a compound (Cpd) salt selected from the consisting of:

| Cpd | Name |
|---|---|
| 357 | 2-[4,6-bis(trifluoromethyl)pyrimidin-2-yl]-6-chloro-1-[6-(morpholin-4-yl)pyridin-3-yl]- |
| | 2,3,4,9-tetrahydro-1H-pyrido[3,4-b]indole formate (1:1); and, |
| 358 | 2-[4,6-bis(trifluoromethyl)pyrimidin-2-yl]-6-chloro-1-[2-(morpholin-4-yl)pyrimidin-5- |
| | yl]-2,3,4,9-tetrahydro-1H-pyrido[3,4-b]indole formate (1:1); |

wherein the form of the compound is selected from the group consisting of a hydrate, solvate, racemate, enantiomer, diastereomer, stereoisomer, and tautomer form thereof.

One aspect of the compound of Formula (I) or a form thereof includes a compound (Cpd) having dihydroorotate dehydrogenase inhibition activity ≤10.0 nM selected from the group consisting of (wherein ¹Cpd #indicates the compound was isolated as a salt form):

| Cpd | Name |
|---|---|
| 7 | 6-chloro-2-(4,6-dichloropyrimidin-2-yl)-1-(4-methylphenyl)-2,3,4,9-tetrahydro-1H- |
| | pyrido[3,4-b]indole; |
| 10 | 6-chloro-2-(4-chloropyrimidin-2-yl)-1-(4-methylphenyl)-2,3,4,9-tetrahydro-1H- |
| | pyrido[3,4-b]indole; |
| 16 | 6-chloro-2-(4,6-dichloropyrimidin-2-yl)-7-fluoro-1-(4-methylphenyl)-2,3,4,9- |
| | tetrahydro-1H-pyrido[3,4-b]indole; |
| 21 | 6-chloro-2-(5-chloropyrimidin-2-yl)-5-fluoro-1-(4-methylphenyl)-2,3,4,9-tetrahydro- |
| | 1H-pyrido[3,4-b]indole; |
| 28 | 6-chloro-2-(4-chloropyrimidin-2-yl)-5-fluoro-1-(4-methylphenyl)-2,3,4,9-tetrahydro- |
| | 1H-pyrido[3,4-b]indole; |
| 43 | 6-chloro-2-(4-chloropyrimidin-2-yl)-7-fluoro-1-(4-methylphenyl)-2,3,4,9-tetrahydro- |
| | 1H-pyrido[3,4-b]indole; |
| 46 | 6-chloro-2-(4-chloro-6-methylpyrimidin-2-yl)-7-fluoro-1-(4-fluorophenyl)-2,3,4,9- |
| | tetrahydro-1H-pyrido[3,4-b]indole; |
| 53 | 6-chloro-2-(4-chloropyrimidin-2-yl)-7-fluoro-1-[4-(propan-2-yl)phenyl]-2,3,4,9- |
| | tetrahydro-1H-pyrido[3,4-b]indole; |
| 60 | 6-chloro-5-fluoro-1-(4-fluorophenyl)-2-[4-(trifluoromethyl)pyrimidin-2-yl]-2,3,4,9- |
| | tetrahydro-1H-pyrido[3,4-b]indole; |
| 66 | 6-chloro-2-(4,6-dimethylpyrimidin-2-yl)-7-fluoro-1-(4-methylphenyl)-2,3,4,9- |
| | tetrahydro-1H-pyrido[3,4-b]indole; |
| 68 | 2-[6-chloro-1-(2,3-difluorophenyl)-7-fluoro-1,3,4,9-tetrahydro-2H-pyrido[3,4-b]indol- |
| | 2-yl]pyrimidine-4-carbonitrile; |
| 70 | 6-chloro-1-(4-chlorophenyl)-5-fluoro-2-(5-fluoro-4-methylpyrimidin-2-yl)-2,3,4,9- |
| | tetrahydro-1H-pyrido[3,4-b]indole; |
| 72 | 6-chloro-7-fluoro-1-(4-methylphenyl)-2-[4-(trifluoromethyl)pyrimidin-2-yl]-2,3,4,9- |
| | tetrahydro-1H-pyrido[3,4-b]indole; |
| 73 | 6-chloro-1-(4-chlorophenyl)-5-fluoro-2-[4-(trifluoromethyl)pyrimidin-2-yl]-2,3,4,9- |
| | tetrahydro-1H-pyrido[3,4-b]indole; |
| 74 | 6-chloro-5-fluoro-1-[4-(trifluoromethyl)phenyl]-2-[4-(trifluoromethyl)pyrimidin-2- |
| | yl]-2,3,4,9-tetrahydro-1H-pyrido[3,4-b]indole; |
| 76 | 6-chloro-5-fluoro-1-(4-methylphenyl)-2-[4-(trifluoromethyl)pyrimidin-2-yl]-2,3,4,9- |
| | tetrahydro-1H-pyrido[3,4-b]indole; |
| 77 | 6-chloro-2-(4-chloropyrimidin-2-yl)-1-(2,3-difluorophenyl)-7-fluoro-2,3,4,9- |
| | tetrahydro-1H-pyrido[3,4-b]indole; |
| 87 | methyl 2-[6-chloro-1-(4-chlorophenyl)-5-fluoro-1,3,4,9-tetrahydro-2H-pyrido[3,4- |
| | b]indol-2-yl]pyrimidine-4-carboxylate; |
| 88 | 4-{6-chloro-7-fluoro-2-[4-methyl-6-(trifluoromethyl)pyrimidin-2-yl]-2,3,4,9- |
| | tetrahydro-1H-pyrido[3,4-b]indol-1-yl}benzonitrile; |
| 89 | 4-{6-chloro-5-fluoro-2-[4-(trifluoromethyl)pyrimidin-2-yl]-2,3,4,9-tetrahydro-1H- |
| | pyrido[3,4-b]indol-1-yl}benzonitrile; |
| 93 | 4-[6-chloro-2-(4,6-dimethylpyrimidin-2-yl)-7-fluoro-2,3,4,9-tetrahydro-1H- |
| | pyrido[3,4-b]indol-1-yl]benzonitrile; |
| 95 | 5-{6-chloro-7-fluoro-2-[4-(trifluoromethyl)pyrimidin-2-yl]-2,3,4,9-tetrahydro-1H- |
| | pyrido[3,4-b]indol-1-yl}-N,N-dimethylpyrimidin-2-amine; |
| 96 | 6-chloro-7-fluoro-1-(4-methylphenyl)-2-[4-methyl-6-(trifluoromethyl)pyrimidin-2- |
| | yl]-2,3,4,9-tetrahydro-1H-pyrido[3,4-b]indole; |
| 100 | 4-{6-chloro-7-fluoro-2-[4-(trifluoromethyl)pyrimidin-2-yl]-2,3,4,9-tetrahydro-1H- |
| | pyrido[3,4-b]indol-1-yl}benzonitrile; |
| 106 | 6-chloro-7-fluoro-2-(5-fluoro-4,6-dimethylpyrimidin-2-yl)-1-(4-methylphenyl)- |
| | 2,3,4,9-tetrahydro-1H-pyrido[3,4-b]indole; |
| 108 | 6-chloro-1-(2,3-difluorophenyl)-7-fluoro-2-[4-(trifluoromethyl)pyrimidin-2-yl]- |
| | 2,3,4,9-tetrahydro-1H-pyrido[3,4-b]indole; |
| 109 | 6-chloro-5-fluoro-2-(5-fluoro-4-methylpyrimidin-2-yl)-1-(4-methylphenyl)-2,3,4,9- |
| | tetrahydro-1H-pyrido[3,4-b]indole; |
| 110 | 2-[6-chloro-5-fluoro-1-(4-methylphenyl)-1,3,4,9-tetrahydro-2H-pyrido[3,4-b]indol-2- |
| | yl]-6-methylpyrimidine-4-carbonitrile; |
| 111 | 2-[6-chloro-5-fluoro-1-(4-fluorophenyl)-1,3,4,9-tetrahydro-2H-pyrido[3,4-b]indol-2- |
| | yl]-6-methylpyrimidine-4-carbonitrile; |
| 112 | 6-chloro-1-(2,3-difluorophenyl)-7-fluoro-2-(5-fluoro-4,6-dimethylpyrimidin-2-yl)- |
| | 2,3,4,9-tetrahydro-1H-pyrido[3,4-b]indole; |
| 113 | 6-chloro-1-(2,3-difluorophenyl)-5-fluoro-2-(5-fluoro-4-methylpyrimidin-2-yl)-2,3,4,9- |
| | tetrahydro-1H-pyrido[3,4-b]indole; |
| 114 | 6-chloro-1-(2,3-difluoro-4-methylphenyl)-5-fluoro-2-(5-fluoro-4-methylpyrimidin-2- |
| | yl)-2,3,4,9-tetrahydro-1H-pyrido[3,4-b]indole |
| 115 | 4-{6-chloro-7-fluoro-2-[4-(trifluoromethyl)pyrimidin-2-yl]-2,3,4,9-tetrahydro-1H- |
| | pyrido[3,4-b]indol-1-yl}benzene-1-sulfonamide; |
| 116 | 6-chloro-5-fluoro-2-(5-fluoro-4,6-dimethylpyrimidin-2-yl)-1-(4-methylphenyl)- |
| | 2,3,4,9-tetrahydro-1H-pyrido[3,4-b]indole; |
| 117 | 6-chloro-1-(2,3-difluorophenyl)-5-fluoro-2-(5-fluoro-4,6-dimethylpyrimidin-2-yl)- |
| | 2,3,4,9-tetrahydro-1H-pyrido[3,4-b]indole; |
| 118 | 6-chloro-1-(2,3-difluoro-4-methylphenyl)-5-fluoro-2-(5-fluoro-4,6- |
| | dimethylpyrimidin-2-yl)-2,3,4,9-tetrahydro-1H-pyrido[3,4-b]indole; |
| 119 | 6-chloro-1-(2,3-difluorophenyl)-2-[4-(trifluoromethyl)pyrimidin-2-yl]-2,3,4,9- |
| | tetrahydro-1H-pyrido[3,4-b]indole; |
| 120 | 6-chloro-1-(2,3-difluoro-4-methoxyphenyl)-2-[4-(trifluoromethyl)pyrimidin-2-yl]- |
| | 2,3,4,9-tetrahydro-1H-pyrido[3,4-b]indole; |
| 122 | 5-{6-chloro-7-fluoro-2-[4-(trifluoromethyl)pyrimidin-2-yl]-2,3,4,9-tetrahydro-1H- |
| | pyrido[3,4-b]indol-1-yl}-N,N-dimethylpyridin-2-amine; |
| 124 | 6-chloro-1-(2,3-difluorophenyl)-7-fluoro-2-(5-fluoro-4-methylpyrimidin-2-yl)-2,3,4,9- |
| | tetrahydro-1H-pyrido[3,4-b]indole; |
| 125 | 6-chloro-1-(2,3-difluoro-4-methylphenyl)-7-fluoro-2-(5-fluoro-4-methylpyrimidin-2- |
| | yl)-2,3,4,9-tetrahydro-1H-pyrido[3,4-b]indole; |
| 128 | 6-chloro-1-(2,3-difluoro-4-methylphenyl)-2-[4-(trifluoromethyl)pyrimidin-2-yl]- |
| | 2,3,4,9-tetrahydro-1H-pyrido[3,4-b]indole; |
| 129 | 6-chloro-1-(2,3-difluorophenyl)-2-(4,6-dimethylpyrimidin-2-yl)-7-fluoro-2,3,4,9- |
| | tetrahydro-1H-pyrido[3,4-b]indole; |
| 130 | 6-chloro-2-(5-chloropyrimidin-2-yl)-1-(2,3-difluoro-4-methylphenyl)-7-fluoro- |
| | 2,3,4,9-tetrahydro-1H-pyrido[3,4-b]indole; |
| 131 | 6-chloro-2-(5-chloropyrimidin-2-yl)-1-(2,3-difluorophenyl)-7-fluoro-2,3,4,9- |
| | tetrahydro-1H-pyrido[3,4-b]indole; |
| 132 | 6-chloro-1-(2,3-difluoro-4-methylphenyl)-2-(4,6-dimethylpyrimidin-2-yl)-7-fluoro- |
| | 2,3,4,9-tetrahydro-1H-pyrido[3,4-b]indole; |
| 136 | 6-chloro-1-(2,3-difluoro-4-methylphenyl)-7-fluoro-2-(5-methylpyrimidin-2-yl)- |
| | 2,3,4,9-tetrahydro-1H-pyrido[3,4-b]indole; |
| 137 | 6-chloro-1-(2,3-difluoro-4-methylphenyl)-5-fluoro-2-(5-methylpyrimidin-2-yl)- |
| | 2,3,4,9-tetrahydro-1H-pyrido[3,4-b]indole; |
| 138 | 6-chloro-1-(2,3-difluoro-4-methylphenyl)-7-fluoro-2-(5-fluoropyrimidin-2-yl)-2,3,4,9- |
| | tetrahydro-1H-pyrido[3,4-b]indole; |
| 139 | 6-chloro-1-(2,3-difluoro-4-methylphenyl)-7-fluoro-2-(4-methylpyrimidin-2-yl)- |
| | 2,3,4,9-tetrahydro-1H-pyrido[3,4-b]indole; |
| 140 | 6-chloro-1-(2,3-difluoro-4-methylphenyl)-7-fluoro-2-[4-(trifluoromethyl)pyrimidin-2- |
| | yl]-2,3,4,9-tetrahydro-1H-pyrido[3,4-b]indole; |
| 141 | 2-[6-chloro-1-(2,3-difluoro-4-methylphenyl)-7-fluoro-1,3,4,9-tetrahydro-2H- |
| | pyrido[3,4-b]indol-2-yl]pyrimidine-4-carbonitrile; |
| 142 | 6-chloro-1-(2,3-difluoro-4-methylphenyl)-7-fluoro-2-[5-(trifluoromethyl)pyrimidin-2- |
| | yl]-2,3,4,9-tetrahydro-1H-pyrido[3,4-b]indole; |
| 143 | 6-chloro-1-(2,3-difluoro-4-methylphenyl)-7-fluoro-2-(5-fluoro-4,6- |
| | dimethylpyrimidin-2-yl)-2,3,4,9-tetrahydro-1H-pyrido[3,4-b]indole; |
| 144 | 6-chloro-1-(4-chloro-2,3-difluorophenyl)-2-[4-(trifluoromethyl)pyrimidin-2-yl]- |
| | 2,3,4,9-tetrahydro-1H-pyrido[3,4-b]indole; |
| 147 | 6-chloro-1-(2,3-difluorophenyl)-7-fluoro-2-[4-methyl-6-(trifluoromethyl)pyrimidin-2- |
| | yl]-2,3,4,9-tetrahydro-1H-pyrido[3,4-b]indole; |
| 148 | 6-chloro-1-(2,3-difluoro-4-methylphenyl)-7-fluoro-2-[4-methyl-6- |
| | (trifluoromethyl)pyrimidin-2-yl]-2,3,4,9-tetrahydro-1H-pyrido[3,4-b]indole; |
| 149 | 6-chloro-5-fluoro-1-[4-(methanesulfonyl)phenyl]-2-[4-(trifluoromethyl)pyrimidin-2- |
| | yl]-2,3,4,9-tetrahydro-1H-pyrido[3,4-b]indole; |
| 150 | 4-{6-chloro-5-fluoro-2-[4-(trifluoromethyl)pyrimidin-2-yl]-2,3,4,9-tetrahydro-1H- |
| | pyrido[3,4-b]indol-1-yl}benzene-1-sulfonamide; |
| 151 | 2-[6-chloro-7-fluoro-1-(4-methylphenyl)-1,3,4,9-tetrahydro-2H-pyrido[3,4-b]indol-2- |
| | yl]-6-methylpyrimidine-4-carbonitrile; |
| 152 | 2-[6-chloro-1-(2,3-difluorophenyl)-7-fluoro-1,3,4,9-tetrahydro-2H-pyrido[3,4-b]indol- |
| | 2-yl]-6-methylpyrimidine-4-carbonitrile; |
| 153 | 2-[6-chloro-1-(2,3-difluoro-4-methylphenyl)-7-fluoro-1,3,4,9-tetrahydro-2H- |
| | pyrido[3,4-b]indol-2-yl]-6-methylpyrimidine-4-carbonitrile; |
| 154 | 2-[4,6-bis(trifluoromethyl)pyrimidin-2-yl]-6-chloro-7-fluoro-1-(4-methylphenyl)- |
| | 2,3,4,9-tetrahydro-1H-pyrido[3,4-b]indole; |
| 155 | 2-[4,6-bis(trifluoromethyl)pyrimidin-2-yl]-6-chloro-1-(2,3-difluoro-4-methylphenyl)- |
| | 7-fluoro-2,3,4,9-tetrahydro-1H-pyrido[3,4-b]indole; |
| 156 | 3-(4-{6-chloro-7-fluoro-2-[4-(trifluoromethyl)pyrimidin-2-yl]-2,3,4,9-tetrahydro-1H- |
| | pyrido[3,4-b]indol-1-yl}phenyl)propan-1-ol; |
| 157 | 2-[4,6-bis(trifluoromethyl)pyrimidin-2-yl]-6-chloro-1-(2,3-difluorophenyl)-7-fluoro- |
| | 2,3,4,9-tetrahydro-1H-pyrido[3,4-b]indole; |
| 158 | 5-{6-chloro-7-fluoro-2-[4-(trifluoromethyl)pyrimidin-2-yl]-2,3,4,9-tetrahydro-1H- |
| | pyrido[3,4-b]indol-1-yl}-3-fluoro-N,N-dimethylpyridin-2-amine; |
| 160 | 5-{6-chloro-5-fluoro-2-[4-(trifluoromethyl)pyrimidin-2-yl]-2,3,4,9-tetrahydro-1H- |
| | pyrido[3,4-b]indol-1-yl}-3-fluoro-N,N-dimethylpyridin-2-amine; |
| 161 | (1S)-6-chloro-1-(4-methylphenyl)-2-[4-(trifluoromethyl)pyrimidin-2-yl]-2,3,4,9- |
| | tetrahydro-1H-pyrido[3,4-b]indole; |
| 162 | 2-[6-chloro-1-(2,3-difluoro-4-methylphenyl)-7-fluoro-1,3,4,9-tetrahydro-2H- |
| | pyrido[3,4-b]indol-2-yl]-5-fluoropyrimidine-4-carbonitrile; |
| 164 | methyl 3-(4-{6-chloro-7-fluoro-2-[4-(trifluoromethyl)pyrimidin-2-yl]-2,3,4,9- |
| | tetrahydro-1H-pyrido[3,4-b]indol-1-yl}phenyl)propanoate; |
| 166 | (4-{6-chloro-7-fluoro-2-[4-(trifluoromethyl)pyrimidin-2-yl]-2,3,4,9-tetrahydro-1H- |
| | pyrido[3,4-b]indol-1-yl}phenyl)methanol; |
| 167 | (3E)-4-(4-{6-chloro-7-fluoro-2-[4-(trifluoromethyl)pyrimidin-2-yl]-2,3,4,9- |
| | tetrahydro-1H-pyrido[3,4-b]indol-1-yl}phenyl)but-3-en-1-ol; |
| 168 | 4-(4-{6-chloro-7-fluoro-2-[4-(trifluoromethyl)pyrimidin-2-yl]-2,3,4,9-tetrahydro-1H- |
| | pyrido[3,4-b]indol-1-yl}phenyl)butan-1-ol; |
| 169 | 6-chloro-1-(2,3-difluoro-4-methylphenyl)-7-fluoro-2-(4,5,6-trimethylpyrimidin-2-yl)- |
| | 2,3,4,9-tetrahydro-1H-pyrido[3,4-b]indole; |
| 171 | 6-chloro-1-(2,3-difluorophenyl)-7-fluoro-2-(4,5,6-trimethylpyrimidin-2-yl)-2,3,4,9- |
| | tetrahydro-1H-pyrido[3,4-b]indole; |
| 172 | 1-(4-bromo-2,3-difluorophenyl)-6-chloro-7-fluoro-2-[4-(trifluoromethyl)pyrimidin-2- |
| | yl]-2,3,4,9-tetrahydro-1H-pyrido[3,4-b]indole; |
| 173 | 4-{6-chloro-7-fluoro-2-[4-(trifluoromethyl)pyrimidin-2-yl]-2,3,4,9-tetrahydro-1H- |
| | pyrido[3,4-b]indol-1-yl}-2,3-difluoro-N,N-dimethylaniline; |
| 174 | 5-fluoro-6-methyl-1-(4-methylphenyl)-2-[4-(trifluoromethyl)pyrimidin-2-yl]-2,3,4,9- |
| | tetrahydro-1H-pyrido[3,4-b]indole; |
| 175 | 2-[6-chloro-7-fluoro-1-(4-methylphenyl)-1,3,4,9-tetrahydro-2H-pyrido[3,4-b]indol-2- |
| | yl]-5-fluoropyrimidine-4-carbonitrile; |
| 177 | 5-{6-chloro-5-fluoro-2-[4-(trifluoromethyl)pyrimidin-2-yl]-2,3,4,9-tetrahydro-1H- |
| | pyrido[3,4-b]indol-1-yl}-N,N-dimethylpyridin-2-amine; |
| 178 | 5-{6-chloro-5-fluoro-2-[4-(trifluoromethyl)pyrimidin-2-yl]-2,3,4,9-tetrahydro-1H- |
| | pyrido[3,4-b]indol-1-yl}-N,N-dimethylpyrimidin-2-amine; |
| 182 | (1S)-6-chloro-1-(4-chlorophenyl)-5-fluoro-2-[4-(trifluoromethyl)pyrimidin-2-yl]- |
| | 2,3,4,9-tetrahydro-1H-pyrido[3,4-b]indole; |
| 183 | 2-[6-chloro-1-(2,3-difluorophenyl)-7-fluoro-1,3,4,9-tetrahydro-2H-pyrido[3,4-b]indol- |
| | 2-yl]-5-fluoropyrimidine-4-carbonitrile; |
| 184 | 6-chloro-1-(2,6-difluoropyridin-3-yl)-7-fluoro-2-[4-(trifluoromethyl)pyrimidin-2-yl]- |
| | 2,3,4,9-tetrahydro-1H-pyrido[3,4-b]indole; |
| 186 | 6-chloro-1-(2,6-difluoropyridin-3-yl)-5-fluoro-2-[4-(trifluoromethyl)pyrimidin-2-yl]- |
| | 2,3,4,9-tetrahydro-1H-pyrido[3,4-b]indole; |
| 188 | 4-{2-[4,6-bis(trifluoromethyl)pyrimidin-2-yl]-6-chloro-7-fluoro-2,3,4,9-tetrahydro- |
| | 1H-pyrido[3,4-b]indol-1-yl}benzonitrile; |
| 189 | (1S)-6-chloro-1-(2,3-difluoro-4-methylphenyl)-5-fluoro-2-(5-fluoro-4,6- |
| | dimethylpyrimidin-2-yl)-2,3,4,9-tetrahydro-1H-pyrido[3,4-b]indole; |
| 192 | 1-(2,3-difluoro-4-methylphenyl)-5-fluoro-6-methyl-2-[4-(trifluoromethyl)pyrimidin-2- |
| | yl]-2,3,4,9-tetrahydro-1H-pyrido[3,4-b]indole; |
| 193 | 6-chloro-5-fluoro-1-[2-(1H-imidazol-1-yl)pyrimidin-5-yl]-2-[4- |
| | (trifluoromethyl)pyrimidin-2-yl]-2,3,4,9-tetrahydro-1H-pyrido[3,4-b]indole; |
| 195 | 6-chloro-2-(5-chloropyrimidin-2-yl)-1-(2,3-difluoro-4-methylphenyl)-5-fluoro- |
| | 2,3,4,9-tetrahydro-1H-pyrido[3,4-b]indole; |
| 196 | 6-chloro-1-(2,3-difluoro-4-methylphenyl)-5-fluoro-2-(4-methylpyrimidin-2-yl)- |
| | 2,3,4,9-tetrahydro-1H-pyrido[3,4-b]indole; |
| 197 | 6-chloro-1-(2,3-difluoro-4-methylphenyl)-5-fluoro-2-[4-(trifluoromethyl)pyrimidin-2- |
| | yl]-2,3,4,9-tetrahydro-1H-pyrido[3,4-b]indole; |
| 198 | 2-[6-chloro-1-(2,3-difluoro-4-methylphenyl)-5-fluoro-1,3,4,9-tetrahydro-2H- |
| | pyrido[3,4-b]indol-2-yl]pyrimidine-4-carbonitrile; |
| 199 | 5-{6-chloro-5-fluoro-2-[4-(trifluoromethyl)pyrimidin-2-yl]-2,3,4,9-tetrahydro-1H- |
| | pyrido[3,4-b]indol-1-yl}-N-methylpyrimidin-2-amine; |
| 200 | 6-chloro-5-fluoro-1-[2-(morpholin-4-yl)pyrimidin-5-yl]-2-[4- |
| | (trifluoromethyl)pyrimidin-2-yl]-2,3,4,9-tetrahydro-1H-pyrido[3,4-b]indole; |
| 201 | 5-[6-chloro-5-fluoro-2-(5-fluoropyrimidin-2-yl)-2,3,4,9-tetrahydro-1H-pyrido[3,4- |
| | b]indol-1-yl]-N,N-dimethylpyrimidin-2-amine; |
| 203 | 6-chloro-1-(2,3-difluoro-4-methylphenyl)-2-(4-methylpyrimidin-2-yl)-2,3,4,9- |
| | tetrahydro-1H-pyrido[3,4-b]indole; |
| 205 | 6-chloro-1-(2,3-difluoro-4-methylphenyl)-2-(4,6-dimethylpyrimidin-2-yl)-5-fluoro- |
| | 2,3,4,9-tetrahydro-1H-pyrido[3,4-b]indole; |
| 206 | 6-chloro-1-(2,3-difluoro-4-methylphenyl)-5-fluoro-2-[4-methyl-6- |
| | (trifluoromethyl)pyrimidin-2-yl]-2,3,4,9-tetrahydro-1H-pyrido[3,4-b]indole; |
| 207 | 6-chloro-1-(4-methylphenyl)-2-[4-(trifluoromethyl)pyrimidin-2-yl]-2,3,4,9-tetrahydro- |
| | 1H-pyrido[3,4-b]indol-8-ol; |
| 208 | 5-{6-chloro-7-fluoro-2-[4-(trifluoromethyl)pyrimidin-2-yl]-2,3,4,9-tetrahydro-1H- |
| | pyrido[3,4-b]indol-1-yl}-6-fluoro-N,N-dimethylpyridin-2-amine; |
| 209 | 2-[4,6-bis(trifluoromethyl)pyrimidin-2-yl]-6-chloro-1-(2,3-difluoro-4-methylphenyl)- |
| | 5-fluoro-2,3,4,9-tetrahydro-1H-pyrido[3,4-b]indole; |
| 210 | 5-{6-chloro-5-fluoro-2-[4-(trifluoromethyl)pyrimidin-2-yl]-2,3,4,9-tetrahydro-1H- |
| | pyrido[3,4-b]indol-1-yl}-6-fluoro-N,N-dimethylpyridin-2-amine; |
| 211 | 6-chloro-5-fluoro-2-(5-fluoropyrimidin-2-yl)-1-[2-(morpholin-4-yl)pyrimidin-5-yl]- |
| | 2,3,4,9-tetrahydro-1H-pyrido[3,4-b]indole; |
| 214 | 2-[6-chloro-1-(2,3-difluoro-4-methylphenyl)-5-fluoro-1,3,4,9-tetrahydro-2H- |
| | pyrido[3,4-b]indol-2-yl]-5-fluoropyrimidine-4-carbonitrile; |
| 215 | 2-[6-chloro-7-fluoro-1-(4-methylphenyl)-1,3,4,9-tetrahydro-2H-pyrido[3,4-b]indol-2- |
| | yl]pyrimidine-4,6-dicarbonitrile; |
| 216 | 2-[6-chloro-1-(2,3-difluoro-4-methylphenyl)-7-fluoro-1,3,4,9-tetrahydro-2H- |
| | pyrido[3,4-b]indol-2-yl]pyrimidine-4,6-dicarbonitrile; |
| 217 | 2-[6-chloro-5-fluoro-1-(4-methylphenyl)-1,3,4,9-tetrahydro-2H-pyrido[3,4-b]indol-2- |
| | yl]-5-fluoropyrimidine-4-carbonitrile; |
| 218 | 2-chloro-5-{6-chloro-2-[4-(trifluoromethyl)pyrimidin-2-yl]-2,3,4,9-tetrahydro-1H- |
| | pyrido[3,4-b]indol-1-yl}phenol; |
| 220 | 6-chloro-1-(2,3-difluoro-4-methylphenyl)-2-[4-(trifluoromethyl)pyrimidin-2-yl]- |
| | 2,3,4,9-tetrahydro-1H-pyrido[3,4-b]indol-8-ol; |
| 221 | 5-chloro-2-[6-chloro-2-(5-fluoropyrimidin-2-yl)-2,3,4,9-tetrahydro-1H-pyrido[3,4- |
| | b]indol-1-yl]phenol; |
| 222 | 5-chloro-2-{6-chloro-2-[4-(trifluoromethyl)pyrimidin-2-yl]-2,3,4,9-tetrahydro-1H- |
| | pyrido[3,4-b]indol-1-yl}phenol; |
| 223 | 2-[(4-{6-chloro-7-fluoro-2-[4-(trifluoromethyl)pyrimidin-2-yl]-2,3,4,9-tetrahydro-1H- |
| | pyrido[3,4-b]indol-1-yl}phenyl)methoxy]ethan-1-ol; |
| 224 | 2-(6-chloro-7-fluoro-1-{4-[(1E)-4-hydroxybut-1-en-1-yl]phenyl}-1,3,4,9-tetrahydro- |
| | 2H-pyrido[3,4-b]indol-2-yl)pyrimidine-4-carbonitrile; |
| 228 | 2-[6-chloro-5-fluoro-1-(4-methylphenyl)-1,3,4,9-tetrahydro-2H-pyrido[3,4-b]indol-2- |
| | yl]pyrimidine-4,6-dicarbonitrile; |
| 229 | 2-[6-chloro-1-(2,3-difluoro-4-methylphenyl)-5-fluoro-1,3,4,9-tetrahydro-2H- |
| | pyrido[3,4-b]indol-2-yl]pyrimidine-4,6-dicarbonitrile; |
| 230 | 6-chloro-5-fluoro-1-[2-(1H-1,2,4-triazol-1-yl)pyrimidin-5-yl]-2-[4- |
| | (trifluoromethyl)pyrimidin-2-yl]-2,3,4,9-tetrahydro-1H-pyrido[3,4-b]indole; |
| 231 | 6-chloro-1-(4-methylphenyl)-2-[4-(trifluoromethyl)pyrimidin-2-yl]-2,3,4,9-tetrahydro- |
| | 1H-pyrido[3,4-b]indol-8-amine; |
| 232 | 2-[6-chloro-1-(2,3-difluoro-4-methylphenyl)-5-fluoro-1,3,4,9-tetrahydro-2H- |
| | pyrido[3,4-b]indol-2-yl]-6-methylpyrimidine-4-carbonitrile; |
| 233 | (1S)-6-chloro-1-(2,3-difluoro-4-methylphenyl)-2-[4-(trifluoromethyl)pyrimidin-2-yl]- |
| | 2,3,4,9-tetrahydro-1H-pyrido[3,4-b]indole; |
| 234 | (1S)-6-chloro-1-(2,3-difluoro-4-methylphenyl)-2-(4-methylpyrimidin-2-yl)-2,3,4,9- |
| | tetrahydro-1H-pyrido[3,4-b]indole; |
| 235 | 6-chloro-1-(2,3-difluoro-4-methylphenyl)-2-(5-fluoro-4-methylpyrimidin-2-yl)- |
| | 2,3,4,9-tetrahydro-1H-pyrido[3,4-b]indole; |
| 236 | 2-(6-chloro-5-fluoro-1-{4-[(1E)-4-hydroxybut-1-en-1-yl]phenyl}-1,3,4,9-tetrahydro- |
| | 2H-pyrido[3,4-b]indol-2-yl)pyrimidine-4-carbonitrile; |
| 237 | (3E)-4-{4-[6-chloro-5-fluoro-2-(4-methylpyrimidin-2-yl)-2,3,4,9-tetrahydro-1H- |
| | pyrido[3,4-b]indol-1-yl]phenyl}but-3-en-1-ol; |
| 238 | (1S)-6-chloro-1-(2,3-difluoro-4-methylphenyl)-2-(5-fluoropyrimidin-2-yl)-2,3,4,9- |
| | tetrahydro-1H-pyrido[3,4-b]indole; |
| 239 | (3E)-4-{4-[6-chloro-7-fluoro-2-(5-fluoro-4-methylpyrimidin-2-yl)-2,3,4,9-tetrahydro- |
| | 1H-pyrido[3,4-b]indol-1-yl]phenyl}but-3-en-1-ol; |
| 240 | 4-{4-[6-chloro-7-fluoro-2-(5-fluoro-4-methylpyrimidin-2-yl)-2,3,4,9-tetrahydro-1H- |
| | pyrido[3,4-b]indol-1-yl]phenyl}butan-1-ol; |
| 241 | (3E)-4-{4-[6-chloro-5-fluoro-2-(5-fluoro-4-methylpyrimidin-2-yl)-2,3,4,9-tetrahydro- |
| | 1H-pyrido[3,4-b]indol-1-yl]phenyl}but-3-en-1-ol; |
| 242 | (3E)-4-{4-[6-chloro-2-(4,6-dimethylpyrimidin-2-yl)-5-fluoro-2,3,4,9-tetrahydro-1H- |
| | pyrido[3,4-b]indol-1-yl]phenyl}but-3-en-1-ol; |
| 243 | (3E)-4-{4-[6-chloro-5-fluoro-2-(5-fluoro-4,6-dimethylpyrimidin-2-yl)-2,3,4,9- |
| | tetrahydro-1H-pyrido[3,4-b]indol-1-yl]phenyl}but-3-en-1-ol; |
| 244 | (3E)-4-{4-[6-chloro-2-(4,6-dimethylpyrimidin-2-yl)-7-fluoro-2,3,4,9-tetrahydro-1H- |
| | pyrido[3,4-b]indol-1-yl]phenyl}but-3-en-1-ol; |
| 245 | 4-{4-[6-chloro-2-(4,6-dimethylpyrimidin-2-yl)-7-fluoro-2,3,4,9-tetrahydro-1H- |
| | pyrido[3,4-b]indol-1-yl]phenyl}butan-1-ol; |
| 246 | 4-{4-[6-chloro-5-fluoro-2-(5-fluoro-4-methylpyrimidin-2-yl)-2,3,4,9-tetrahydro-1H- |
| | pyrido[3,4-b]indol-1-yl]phenyl}butan-1-ol; |
| 247 | 5-bromo-6-chloro-1-(4-chlorophenyl)-2-[4-(trifluoromethyl)pyrimidin-2-yl]-2,3,4,9- |
| | tetrahydro-1H-pyrido[3,4-b]indole; |
| 248 | 6-chloro-1-(2,3-difluoro-4-methylphenyl)-2-[4-(trifluoromethyl)pyrimidin-2-yl]- |
| | 2,3,4,9-tetrahydro-1H-pyrido[3,4-b]indol-8-amine; |
| 249 | 4-{4-[6-chloro-7-fluoro-2-(5-fluoro-4,6-dimethylpyrimidin-2-yl)-2,3,4,9-tetrahydro- |
| | 1H-pyrido[3,4-b]indol-1-yl]phenyl}butan-1-ol; |
| 251 | 6-chloro-1-[2-(morpholin-4-yl)pyrimidin-5-yl]-2-[4-(trifluoromethyl)pyrimidin-2-yl]- |
| | 2,3,4,9-tetrahydro-1H-pyrido[3,4-b]indole; |
| 252 | 2-{6-chloro-2-[4-(trifluoromethyl)pyrimidin-2-yl]-2,3,4,9-tetrahydro-1H-pyrido[3,4- |
| | b]indol-1-yl}-6-fluorophenol; |
| 254 | 6-chloro-1-(2-fluoro-4-methylphenyl)-2-[4-(trifluoromethyl)pyrimidin-2-yl]-2,3,4,9- |
| | tetrahydro-1H-pyrido[3,4-b]indole; |
| 255 | 4-{6-chloro-2-[4-(trifluoromethyl)pyrimidin-2-yl]-2,3,4,9-tetrahydro-1H-pyrido[3,4- |
| | b]indol-1-yl}-3-fluorobenzonitrile; |
| 256 | 3-{6-chloro-2-[4-(trifluoromethyl)pyrimidin-2-yl]-2,3,4,9-tetrahydro-1H-pyrido[3,4- |
| | b]indol-1-yl}-2-fluorophenol; |
| 257 | 2-{6-chloro-2-[4-(trifluoromethyl)pyrimidin-2-yl]-2,3,4,9-tetrahydro-1H-pyrido[3,4- |
| | b]indol-1-yl}-5-methylphenol; |
| 259 | 6-chloro-1-(2-fluoro-3-methylphenyl)-2-[4-(trifluoromethyl)pyrimidin-2-yl]-2,3,4,9- |
| | tetrahydro-1H-pyrido[3,4-b]indole; |
| 260 | 6-chloro-1-[4-(1H-imidazol-1-yl)phenyl]-2-[4-(trifluoromethyl)pyrimidin-2-yl]- |
| | 2,3,4,9-tetrahydro-1H-pyrido[3,4-b]indole; |
| 261 | 6-chloro-1-[4-(1H-pyrazol-1-yl)phenyl]-2-[4-(trifluoromethyl)pyrimidin-2-yl]-2,3,4,9- |
| | tetrahydro-1H-pyrido[3,4-b]indole; |
| 262 | 6-chloro-1-[4-(morpholin-4-yl)phenyl]-2-[4-(trifluoromethyl)pyrimidin-2-yl]-2,3,4,9- |
| | tetrahydro-1H-pyrido[3,4-b]indole; |
| 263 | 6-chloro-2-[4-(trifluoromethyl)pyrimidin-2-yl]-1-(2,3,4-trifluorophenyl)-2,3,4,9- |
| | tetrahydro-1H-pyrido[3,4-b]indole; |
| 265 | 1-(4-methylphenyl)-6-nitro-2-[4-(trifluoromethyl)pyrimidin-2-yl]-2,3,4,9-tetrahydro- |
| | 1H-pyrido[3,4-b]indole; |
| 266 | 6-chloro-1-(2,3-difluoro-4-methylphenyl)-5-nitro-2-[4-(trifluoromethyl)pyrimidin-2- |
| | yl]-2,3,4,9-tetrahydro-1H-pyrido[3,4-b]indole; |
| 267 | 6-chloro-1-(4-chloro-2-fluorophenyl)-2-[4-(trifluoromethyl)pyrimidin-2-yl]-2,3,4,9- |
| | tetrahydro-1H-pyrido[3,4-b]indole; |
| 268 | 1-(4-bromo-2-fluorophenyl)-6-chloro-2-[4-(trifluoromethyl)pyrimidin-2-yl]-2,3,4,9- |
| | tetrahydro-1H-pyrido[3,4-b]indole; |
| 270 | 1-(4-bromo-2,3-difluorophenyl)-6-chloro-2-[4-(trifluoromethyl)pyrimidin-2-yl]- |
| | 2,3,4,9-tetrahydro-1H-pyrido[3,4-b]indole; |
| 271 | 1-(2,3-difluoro-4-methylphenyl)-6-methyl-2-[4-(trifluoromethyl)pyrimidin-2-yl]- |
| | 2,3,4,9-tetrahydro-1H-pyrido[3,4-b]indole; |
| 272 | 1-(2,3-difluoro-4-methylphenyl)-6-methyl-2-[4-(trifluoromethyl)pyrimidin-2-yl]- |
| | 2,3,4,9-tetrahydro-1H-pyrido[3,4-b]indol-8-amine; |
| 273 | 6-chloro-1-(2,3-difluoro-4-methylphenyl)-2-[4-(trifluoromethyl)pyrimidin-2-yl]- |
| | 2,3,4,9-tetrahydro-1H-pyrido[3,4-b]indol-5-amine; |
| 275 | 6-chloro-1-[2-fluoro-4-(1H-imidazol-1-yl)phenyl]-2-[4-(trifluoromethyl)pyrimidin-2- |
| | yl]-2,3,4,9-tetrahydro-1H-pyrido[3,4-b]indole; |
| 276 | 1-(2,3-difluoro-4-methylphenyl)-6-methyl-2-[4-(trifluoromethyl)pyrimidin-2-yl]- |
| | 2,3,4,9-tetrahydro-1H-pyrido[3,4-b]indol-8-ol; |
| 277 | 6-chloro-1-[2-fluoro-4-(morpholin-4-yl)phenyl]-2-[4-(trifluoromethyl)pyrimidin-2- |
| | yl]-2,3,4,9-tetrahydro-1H-pyrido[3,4-b]indole; |
| 278 | 4-{6-chloro-2-[4-(trifluoromethyl)pyrimidin-2-yl]-2,3,4,9-tetrahydro-1H-pyrido[3,4- |
| | b]indol-1-yl}-2,3-difluorobenzonitrile; |
| 279 | 6-chloro-1-[2,3-difluoro-4-(morpholin-4-yl)phenyl]-2-[4-(trifluoromethyl)pyrimidin- |
| | 2-yl]-2,3,4,9-tetrahydro-1H-pyrido[3,4-b]indole; |
| 280 | butyl 2-[6-chloro-1-(4-methylphenyl)-1,3,4,9-tetrahydro-2H-pyrido[3,4-b]indol-2- |
| | yl]pyrimidine-4-carboxylate; |
| 281 | butyl 2-[6-chloro-1-(2,3-difluoro-4-methylphenyl)-1,3,4,9-tetrahydro-2H-pyrido[3,4- |
| | b]indol-2-yl]pyrimidine-4-carboxylate; |
| 285 | 6-chloro-1-[2,3-difluoro-4-(1H-imidazol-1-yl)phenyl]-2-[4- |
| | (trifluoromethyl)pyrimidin-2-yl]-2,3,4,9-tetrahydro-1H-pyrido[3,4-b]indole; |
| 286 | 6-chloro-2-(4,6-dimethylpyrimidin-2-yl)-1-(4-methylphenyl)-2,3,4,9-tetrahydro-1H- |
| | pyrido[3,4-b]indole; |
| 287 | 1-(4-methylphenyl)-6-(2H-1,2,3-triazol-2-yl)-2-[4-(trifluoromethyl)pyrimidin-2-yl]- |
| | 2,3,4,9-tetrahydro-1H-pyrido[3,4-b]indole; |
| 288 | 1-[2-(morpholin-4-yl)pyrimidin-5-yl]-6-(2H-1,2,3-triazol-2-yl)-2-[4- |
| | (trifluoromethyl)pyrimidin-2-yl]-2,3,4,9-tetrahydro-1H-pyrido[3,4-b]indole; |
| 291 | 6-ethenyl-1-(4-methylphenyl)-2-[4-(trifluoromethyl)pyrimidin-2-yl]-2,3,4,9- |
| | tetrahydro-1H-pyrido[3,4-b]indole; |
| 298 | 6-bromo-1-[2-(morpholin-4-yl)pyrimidin-5-yl]-2-[4-(trifluoromethyl)pyrimidin-2-yl]- |
| | 2,3,4,9-tetrahydro-1H-pyrido[3,4-b]indole; |
| 302 | 6-methoxy-1-[2-(morpholin-4-yl)pyrimidin-5-yl]-2-[4-(trifluoromethyl)pyrimidin-2- |
| | yl]-2,3,4,9-tetrahydro-1H-pyrido[3,4-b]indole; |
| 303 | 6-chloro-1-[6-(morpholin-4-yl)pyridin-3-yl]-2-[4-(trifluoromethyl)pyrimidin-2-yl]- |
| | 2,3,4,9-tetrahydro-1H-pyrido[3,4-b]indole; |
| 305 | 1-(2,3-difluoro-4-methylphenyl)-6-nitro-2-[4-(trifluoromethyl)pyrimidin-2-yl]- |
| | 2,3,4,9-tetrahydro-1H-pyrido[3,4-b]indole; |
| 308 | 6-chloro-2-(4,6-dimethylpyrimidin-2-yl)-1-[2-(morpholin-4-yl)pyrimidin-5-yl]- |
| | 2,3,4,9-tetrahydro-1H-pyrido[3,4-b]indole; |
| 310 | 6-chloro-1-[2-(morpholin-4-yl)pyrimidin-5-yl]-2-[4-(trifluoromethyl)pyrimidin-2-yl]- |
| | 2,3,4,9-tetrahydro-1H-pyrido[3,4-b]indol-8-amine; |
| 311 | 6-chloro-1-[2-(morpholin-4-yl)pyrimidin-5-yl]-2-[4-(trifluoromethyl)pyrimidin-2-yl]- |
| | 2,3,4,9-tetrahydro-1H-pyrido[3,4-b]indol-7-amine; |
| 312 | 6-chloro-2-(4-methylpyrimidin-2-yl)-1-[2-(morpholin-4-yl)pyrimidin-5-yl]-2,3,4,9- |
| | tetrahydro-1H-pyrido[3,4-b]indole; |
| 316 | 1-[6-(morpholin-4-yl)pyridin-3-yl]-6-(2H-1,2,3-triazol-2-yl)-2-[4- |
| | (trifluoromethyl)pyrimidin-2-yl]-2,3,4,9-tetrahydro-1H-pyrido[3,4-b]indole; |
| 323 | 1-[2-(morpholin-4-yl)pyrimidin-5-yl]-6-nitro-2-[4-(trifluoromethyl)pyrimidin-2-yl]- |
| | 2,3,4,9-tetrahydro-1H-pyrido[3,4-b]indole; |
| 328 | 6-chloro-1-[2-(morpholin-4-yl)pyrimidin-5-yl]-2-[4-(trifluoromethyl)pyrimidin-2-yl]- |
| | 2,3,4,9-tetrahydro-1H-pyrido[3,4-b]indol-5-amine; |
| 335 | 6-chloro-1-[2-(2-oxa-5-azabicyclo[2.2.1]heptan-5-yl)pyrimidin-5-yl]-2-[4- |
| | (trifluoromethyl)pyrimidin-2-yl]-2,3,4,9-tetrahydro-1H-pyrido[3,4-b]indole; |
| 337 | 6-chloro-1-[2-(3-oxa-8-azabicyclo[3.2.1]octan-8-yl)pyrimidin-5-yl]-2-[4- |
| | (trifluoromethyl)pyrimidin-2-yl]-2,3,4,9-tetrahydro-1H-pyrido[3,4-b]indole; |
| 340 | 2-[4,6-bis(trifluoromethyl)pyrimidin-2-yl]-6-chloro-1-(2,3-difluoro-4-methylphenyl)- |
| | 2,3,4,9-tetrahydro-1H-pyrido[3,4-b]indole; |
| 341 | 6-chloro-1-(2-fluorophenyl)-2-[4-(trifluoromethyl)pyrimidin-2-yl]-2,3,4,9-tetrahydro- |
| | 1H-pyrido[3,4-b]indole; |
| 342 | 6-chloro-1-(2,3-difluoro-4-methylphenyl)-2-(4,6-dimethylpyrimidin-2-yl)-2,3,4,9- |
| | tetrahydro-1H-pyrido[3,4-b]indole; |
| 343 | 6-chloro-1-(4-methylphenyl)-2-[4-(trifluoromethyl)pyrimidin-2-yl]-2,3,4,9-tetrahydro- |
| | 1H-pyrido[3,4-b]indole; |
| 349 | 6-chloro-1-(4-chlorophenyl)-2-[4-(trifluoromethyl)pyrimidin-2-yl]-2,3,4,9-tetrahydro- |
| | 1H-pyrido[3,4-b]indole; |
| 350 | 6-chloro-2-(4,6-dimethylpyrimidin-2-yl)-1-[6-(morpholin-4-yl)pyridin-3-yl]-2,3,4,9- |
| | tetrahydro-1H-pyrido[3,4-b]indole; |
| 351 | 6-chloro-2-(4-methylpyrimidin-2-yl)-1-[6-(morpholin-4-yl)pyridin-3-yl]-2,3,4,9- |
| | tetrahydro-1H-pyrido[3,4-b]indol-8-amine; |
| 352 | 6-chloro-2-(4-methylpyrimidin-2-yl)-1-[6-(morpholin-4-yl)pyridin-3-yl]-2,3,4,9- |
| | tetrahydro-1H-pyrido[3,4-b]indole; |
| 353 | 2-{6-chloro-2-[4-(trifluoromethyl)pyrimidin-2-yl]-2,3,4,9-tetrahydro-1H-pyrido[3,4- |
| | b]indol-1-yl}phenol; |
| 354 | 6-chloro-1-(3-fluoro-4-methylphenyl)-2-[4-(trifluoromethyl)pyrimidin-2-yl]-2,3,4,9- |
| | tetrahydro-1H-pyrido[3,4-b]indole; |
| 356 | 2-{6-chloro-1-[6-(morpholin-4-yl)pyridin-3-yl]-1,3,4,9-tetrahydro-2H-pyrido[3,4- |
| | b]indol-2-yl}pyrimidine-4-carbonitrile; |
| ¹357 | 2-[4,6-bis(trifluoromethyl)pyrimidin-2-yl]-6-chloro-1-[6-(morpholin-4-yl)pyridin-3- |
| | yl]-2,3,4,9-tetrahydro-1H-pyrido[3,4-b]indole; |
| ¹358 | 2-[4,6-bis(trifluoromethyl)pyrimidin-2-yl]-6-chloro-1-[2-(morpholin-4-yl)pyrimidin- |
| | 5-yl]-2,3,4,9-tetrahydro-1H-pyrido[3,4-b]indole; |
| 359 | 2-{6-chloro-1-[2-(morpholin-4-yl)pyrimidin-5-yl]-1,3,4,9-tetrahydro-2H-pyrido[3,4- |
| | b]indol-2-yl}pyrimidine-4-carbonitrile; |
| 360 | 2-[4,6-bis(trifluoromethyl)pyrimidin-2-yl]-6-chloro-1-(4-methylphenyl)-2,3,4,9- |
| | tetrahydro-1H-pyrido[3,4-b]indole; |
| 361 | 6-chloro-1-(4-methylphenyl)-2-[4-methyl-6-(trifluoromethyl)pyrimidin-2-yl]-2,3,4,9- |
| | tetrahydro-1H-pyrido[3,4-b]indole; |
| 362 | 2-{6-chloro-1-[2-(morpholin-4-yl)pyrimidin-5-yl]-1,3,4,9-tetrahydro-2H-pyrido[3,4- |
| | b]indol-2-yl}-6-methylpyrimidine-4-carbonitrile; |
| 363 | 2-[6-chloro-1-(2,3-difluoro-4-methylphenyl)-1,3,4,9-tetrahydro-2H-pyrido[3,4- |
| | b]indol-2-yl]pyrimidine-4-carbonitrile; |
| 365 | 6-chloro-2-(5-fluoro-4-methylpyrimidin-2-yl)-1-[6-(morpholin-4-yl)pyridin-3-yl]- |
| | 2,3,4,9-tetrahydro-1H-pyrido[3,4-b]indole; |
| 367 | 6-chloro-2-(5-fluoro-4,6-dimethylpyrimidin-2-yl)-1-[6-(morpholin-4-yl)pyridin-3-yl]- |
| | 2,3,4,9-tetrahydro-1H-pyrido[3,4-b]indole; |
| 368 | 6-chloro-2-(5-fluoro-4,6-dimethylpyrimidin-2-yl)-1-[2-(morpholin-4-yl)pyrimidin-5- |
| | yl]-2,3,4,9-tetrahydro-1H-pyrido[3,4-b]indole; |
| 369 | 6-chloro-1-[2-(4-methylpiperazin-1-yl)pyrimidin-5-yl]-2-[4- |
| | (trifluoromethyl)pyrimidin-2-yl]-2,3,4,9-tetrahydro-1H-pyrido[3,4-b]indole; |
| 371 | 6-chloro-1-(3-fluorophenyl)-2-[4-(trifluoromethyl)pyrimidin-2-yl]-2,3,4,9-tetrahydro- |
| | 1H-pyrido[3,4-b]indole; |
| 372 | 6-chloro-2-[4-methyl-6-(trifluoromethyl)pyrimidin-2-yl]-1-[6-(morpholin-4- |
| | yl)pyridin-3-yl]-2,3,4,9-tetrahydro-1H-pyrido[3,4-b]indole; |
| 373 | 6-chloro-1-{6-[(1R,4R)-2-oxa-5-azabicyclo[2.2.1]heptan-5-yl]pyridin-3-yl}-2-[4- |
| | (trifluoromethyl)pyrimidin-2-yl]-2,3,4,9-tetrahydro-1H-pyrido[3,4-b]indole; |
| 374 | 6-chloro-2-[4-methyl-6-(trifluoromethyl)pyrimidin-2-yl]-1-[2-(morpholin-4- |
| | yl)pyrimidin-5-yl]-2,3,4,9-tetrahydro-1H-pyrido[3,4-b]indole; |
| 375 | 6-chloro-1-{6-[(1S,4S)-2-oxa-5-azabicyclo[2.2.1]heptan-5-yl]pyridin-3-yl}-2-[4- |
| | (trifluoromethyl)pyrimidin-2-yl]-2,3,4,9-tetrahydro-1H-pyrido[3,4-b]indole; |
| 378 | 2-[6-chloro-1-(4-methylphenyl)-1,3,4,9-tetrahydro-2H-pyrido[3,4-b]indol-2- |
| | yl]pyrimidine-4-carbonitrile; |
| 379 | 2-[6-chloro-1-(4-methylphenyl)-1,3,4,9-tetrahydro-2H-pyrido[3,4-b]indol-2-yl]-6- |
| | methylpyrimidine-4-carbonitrile; |
| 381 | 2-[4,6-bis(trifluoromethyl)pyrimidin-2-yl]-6-chloro-1-[2-(4-methylpiperazin-1- |
| | yl)pyrimidin-5-yl]-2,3,4,9-tetrahydro-1H-pyrido[3,4-b]indole; |
| 383 | 6-chloro-1-[2-(morpholin-4-yl)pyrimidin-5-yl]-2-[5-(trifluoromethyl)-1,2,4-triazin-3- |
| | yl]-2,3,4,9-tetrahydro-1H-pyrido[3,4-b]indole; |
| 385 | 2-[(1S)-6-chloro-1-(2,3-difluoro-4-methylphenyl)-1,3,4,9-tetrahydro-2H-pyrido[3,4- |
| | b]indol-2-yl]-5-fluoropyrimidine-4-carbonitrile; |
| 391 | 6-chloro-1-[2-(2-oxa-6-azaspiro[3.3]heptan-6-yl)pyrimidin-5-yl]-2-[4- |
| | (trifluoromethyl)pyrimidin-2-yl]-2,3,4,9-tetrahydro-1H-pyrido[3,4-b]indole; |
| 392 | 2-[(1S)-6-chloro-1-(4-methylphenyl)-1,3,4,9-tetrahydro-2H-pyrido[3,4-b]indol-2-yl]- |
| | 5-fluoropyrimidine-4-carbonitrile; |
| 394 | (1S)-6-chloro-2-(5-fluoro-4-methylpyrimidin-2-yl)-1-(4-methylphenyl)-2,3,4,9- |
| | tetrahydro-1H-pyrido[3,4-b]indole; |
| 395 | (1S)-6-chloro-2-(5-fluoro-4,6-dimethylpyrimidin-2-yl)-1-(4-methylphenyl)-2,3,4,9- |
| | tetrahydro-1H-pyrido[3,4-b]indole; |
| 396 | 2-{(1S)-6-chloro-1-[2-(morpholin-4-yl)pyrimidin-5-yl]-1,3,4,9-tetrahydro-2H- |
| | pyrido[3,4-b]indol-2-yl}-5-fluoropyrimidine-4-carbonitrile; |
| 397 | 2-[(1S)-6-chloro-1-(4-methylphenyl)-1,3,4,9-tetrahydro-2H-pyrido[3,4-b]indol-2- |
| | yl]pyrimidine-4,6-dicarbonitrile; |
| 400 | 2-{(1S)-6-chloro-1-[6-(morpholin-4-yl)pyridin-3-yl]-1,3,4,9-tetrahydro-2H- |
| | pyrido[3,4-b]indol-2-yl}-5-fluoropyrimidine-4-carbonitrile; |
| 401 | 6-chloro-1-(2,3-difluoro-4-methylphenyl)-2-(5-fluoropyrimidin-2-yl)-2,3,4,9- |
| | tetrahydro-1H-pyrido[3,4-b]indole; |
| 403 | 6-chloro-1-(2,3-difluoro-4-methylphenyl)-2-[5-(trifluoromethyl)-1,2,4-triazin-3-yl]- |
| | 2,3,4,9-tetrahydro-1H-pyrido[3,4-b]indole; |
| 407 | (1S)-2-[4,6-bis(trifluoromethyl)pyrimidin-2-yl]-6-chloro-1-(2,3-difluoro-4- |
| | methylphenyl)-2,3,4,9-tetrahydro-1H-pyrido[3,4-b]indole; |
| 408 | (1S)-6-chloro-1-[2-(morpholin-4-yl)pyrimidin-5-yl]-2-[4-(trifluoromethyl)pyrimidin- |
| | 2-yl]-2,3,4,9-tetrahydro-1H-pyrido[3,4-b]indole; |
| 409 | (1S)-2-[4,6-bis(trifluoromethyl)pyrimidin-2-yl]-6-chloro-1-[6-(morpholin-4- |
| | yl)pyridin-3-yl]-2,3,4,9-tetrahydro-1H-pyrido[3,4-b]indole; |
| 410 | (1S)-2-[4,6-bis(trifluoromethyl)pyrimidin-2-yl]-6-chloro-1-[2-(morpholin-4- |
| | yl)pyrimidin-5-yl]-2,3,4,9-tetrahydro-1H-pyrido[3,4-b]indole; |
| 412 | (1S)-6-chloro-1-(2,3-difluoro-4-methylphenyl)-2-(5-fluoro-4,6-dimethylpyrimidin-2- |
| | yl)-2,3,4,9-tetrahydro-1H-pyrido[3,4-b]indole; |
| 413 | 2-[(1S)-6-chloro-1-(2,3-difluoro-4-methylphenyl)-1,3,4,9-tetrahydro-2H-pyrido[3,4- |
| | b]indol-2-yl]-5-fluoro-6-methylpyrimidine-4-carbonitrile; |
| 416 | 2-[(1S)-6-chloro-1-(2,3-difluoro-4-methylphenyl)-1,3,4,9-tetrahydro-2H-pyrido[3,4- |
| | b]indol-2-yl]-6-(trifluoromethyl)pyrimidine-4-carbonitrile; |
| 417 | 2-[(1S)-6-chloro-1-(4-methylphenyl)-1,3,4,9-tetrahydro-2H-pyrido[3,4-b]indol-2-yl]- |
| | 5-fluoro-6-methylpyrimidine-4-carbonitrile; |
| 419 | (1S)-2-[4,6-bis(trifluoromethyl)pyrimidin-2-yl]-6-chloro-1-[2-(4-methylpiperazin-1- |
| | yl)pyrimidin-5-yl]-2,3,4,9-tetrahydro-1H-pyrido[3,4-b]indole; |
| 421 | 2-{(1S)-6-chloro-1-[2-(morpholin-4-yl)pyrimidin-5-yl]-1,3,4,9-tetrahydro-2H- |
| | pyrido[3,4-b]indol-2-yl}-5-fluoro-6-methylpyrimidine-4-carbonitrile; |
| 422 | 6-chloro-1-(4-fluorophenyl)-2-[4-(trifluoromethyl)pyrimidin-2-yl]-2,3,4,9-tetrahydro- |
| | 1H-pyrido[3,4-b]indole; |
| 423 | 6-chloro-1-(2,4-difluorophenyl)-2-[4-(trifluoromethyl)pyrimidin-2-yl]-2,3,4,9- |
| | tetrahydro-1H-pyrido[3,4-b]indole; |
| 424 | 2-{(1S)-6-chloro-1-[6-(morpholin-4-yl)pyridin-3-yl]-1,3,4,9-tetrahydro-2H- |
| | pyrido[3,4-b]indol-2-yl}-5-fluoro-6-methylpyrimidine-4-carbonitrile; |
| 425 | 2-[(1S)-6-chloro-1-(4-methylphenyl)-1,3,4,9-tetrahydro-2H-pyrido[3,4-b]indol-2-yl]- |
| | 6-(trifluoromethyl)pyrimidine-4-carbonitrile; |
| 429 | 6-chloro-1-[6-(morpholin-4-yl)pyridin-3-yl]-2-[5-(trifluoromethyl)-1,2,4-triazin-3-yl]- |
| | 2,3,4,9-tetrahydro-1H-pyrido[3,4-b]indole; |
| 430 | 6-chloro-1-(4-methylphenyl)-2-[5-(trifluoromethyl)-1,2,4-triazin-3-yl]-2,3,4,9- |
| | tetrahydro-1H-pyrido[3,4-b]indole; |
| 431 | 6-chloro-1-(2,3-difluoro-4-methylphenyl)-2-[6-(trifluoromethyl)-1,2,4-triazin-3-yl]- |
| | 2,3,4,9-tetrahydro-1H-pyrido[3,4-b]indole; |
| 432 | 6-chloro-1-[2-fluoro-6-(morpholin-4-yl)pyridin-3-yl]-2-[4-(trifluoromethyl)pyrimidin- |
| | 2-yl]-2,3,4,9-tetrahydro-1H-pyrido[3,4-b]indole; |
| 433 | 2-{(1S)-6-chloro-1-[6-(morpholin-4-yl)pyridin-3-yl]-1,3,4,9-tetrahydro-2H- |
| | pyrido[3,4-b]indol-2-yl}-6-(trifluoromethyl)pyrimidine-4-carbonitrile; |
| 434 | 2-{(1S)-6-chloro-1-[2-(morpholin-4-yl)pyrimidin-5-yl]-1,3,4,9-tetrahydro-2H- |
| | pyrido[3,4-b]indol-2-yl}-6-(trifluoromethyl)pyrimidine-4-carbonitrile; |
| 447 | 4-{6-chloro-2-[4-(trifluoromethyl)pyrimidin-2-yl]-2,3,4,9-tetrahydro-1H-pyrido[3,4- |
| | b]indol-1-yl}benzonitrile; |
| 454 | 2-[4,6-bis(trifluoromethyl)pyrimidin-2-yl]-1-[6-(morpholin-4-yl)pyridin-3-yl]-6-(2H- |
| | 1,2,3-triazol-2-yl)-2,3,4,9-tetrahydro-1H-pyrido[3,4-b]indole; |
| 455 | 2-[4,6-bis(trifluoromethyl)pyrimidin-2-yl]-6-(4-methyl-2H-1,2,3-triazol-2-yl)-1-[6- |
| | (morpholin-4-yl)pyridin-3-yl]-2,3,4,9-tetrahydro-1H-pyrido[3,4-b]indole; |
| 458 | 6-chloro-1-[2-(morpholin-4-yl)pyrimidin-5-yl]-2-[6-(trifluoromethyl)-1,2,4-triazin-3- |
| | yl]-2,3,4,9-tetrahydro-1H-pyrido[3,4-b]indole; |
| 463 | (1S)-2-(5-bromopyrimidin-2-yl)-6-chloro-1-(2,3-difluoro-4-methylphenyl)-2,3,4,9- |
| | tetrahydro-1H-pyrido[3,4-b]indole; |
| 469 | 6-chloro-1-(2,3-difluoro-4-methylphenyl)-2-(4-phenylpyrimidin-2-yl)-2,3,4,9- |
| | tetrahydro-1H-pyrido[3,4-b]indole; |
| 470 | (1S)-2-(5-bromopyrimidin-2-yl)-6-chloro-1-[2-(morpholin-4-yl)pyrimidin-5-yl]- |
| | 2,3,4,9-tetrahydro-1H-pyrido[3,4-b]indole; |
| 471 | (1S)-6-chloro-1-(2,3-difluoro-4-methylphenyl)-2-(5-phenylpyrimidin-2-yl)-2,3,4,9- |
| | tetrahydro-1H-pyrido[3,4-b]indole; |
| 472 | (1S)-6-chloro-1-[2-(morpholin-4-yl)pyrimidin-5-yl]-2-(5-phenylpyrimidin-2-yl)- |
| | 2,3,4,9-tetrahydro-1H-pyrido[3,4-b]indole; |
| 473 | (1S)-6-chloro-1-[6-(morpholin-4-yl)pyridin-3-yl]-2-[4-(trifluoromethyl)pyrimidin-2- |
| | yl]-2,3,4,9-tetrahydro-1H-pyrido[3,4-b]indole; |
| 480 | (1S)-6-chloro-1-[6-(4-methylpiperazin-1-yl)pyridin-3-yl]-2-[4- |
| | (trifluoromethyl)pyrimidin-2-yl]-2,3,4,9-tetrahydro-1H-pyrido[3,4-b]indole; |
| 481 | (1S)-2-[4,6-bis(trifluoromethyl)pyrimidin-2-yl]-6-chloro-1-[6-(4-methylpiperazin-1- |
| | yl)pyridin-3-yl]-2,3,4,9-tetrahydro-1H-pyrido[3,4-b]indole; |
| 482 | 2-[4,6-bis(trifluoromethyl)pyrimidin-2-yl]-1-[2-(morpholin-4-yl)pyrimidin-5-yl]-6- |
| | (2H-1,2,3-triazol-2-yl)-2,3,4,9-tetrahydro-1H-pyrido[3,4-b]indole; |
| 483 | 2-[4,6-bis(trifluoromethyl)pyrimidin-2-yl]-6-(4-methyl-2H-1,2,3-triazol-2-yl)-1-[2- |
| | (morpholin-4-yl)pyrimidin-5-yl]-2,3,4,9-tetrahydro-1H-pyrido[3,4-b]indole; |
| 484 | (1S)-6-chloro-1-[2-(4-methylpiperazin-1-yl)pyrimidin-5-yl]-2-[4- |
| | (trifluoromethyl)pyrimidin-2-yl]-2,3,4,9-tetrahydro-1H-pyrido[3,4-b]indole; |
| 485 | (1S)-2-[4,6-bis(trifluoromethyl)pyrimidin-2-yl]-1-[6-(morpholin-4-yl)pyridin-3-yl]-6- |
| | (2H-1,2,3-triazol-2-yl)-2,3,4,9-tetrahydro-1H-pyrido[3,4-b]indole; |
| 487 | (1S)-2-[4,6-bis(trifluoromethyl)pyrimidin-2-yl]-6-bromo-1-[6-(morpholin-4- |
| | yl)pyridin-3-yl]-2,3,4,9-tetrahydro-1H-pyrido[3,4-b]indole; |
| 490 | 2-[4,6-bis(trifluoromethyl)pyrimidin-2-yl]-1-(2,3-difluoro-4-methylphenyl)-6-fluoro- |
| | 2,3,4,9-tetrahydro-1H-pyrido[3,4-b]indole; |
| 496 | (1S)-2-[4,6-bis(trifluoromethyl)pyrimidin-2-yl]-6-chloro-1-[2-(3,3-dimethylpiperazin- |
| | 1-yl)pyrimidin-5-yl]-2,3,4,9-tetrahydro-1H-pyrido[3,4-b]indole; |
| 499 | (1S)-2-[4,6-bis(trifluoromethyl)pyrimidin-2-yl]-6-bromo-1-[2-(4-methylpiperazin-1- |
| | yl)pyrimidin-5-yl]-2,3,4,9-tetrahydro-1H-pyrido[3,4-b]indole; |
| 500 | (1S)-2-[4,6-bis(trifluoromethyl)pyrimidin-2-yl]-1-[2-(4-methylpiperazin-1- |
| | yl)pyrimidin-5-yl]-6-(2H-1,2,3-triazol-2-yl)-2,3,4,9-tetrahydro-1H-pyrido[3,4- |
| | b]indole; |
| 502 | 1-(2,3-difluoro-4-methylphenyl)-6-(2H-1,2,3-triazol-2-yl)-2-[4- |
| | (trifluoromethyl)pyrimidin-2-yl]-2,3,4,9-tetrahydro-1H-pyrido[3,4-b]indole; |
| 503 | (1S)-2-[4,6-bis(trifluoromethyl)pyrimidin-2-yl]-6-chloro-1-{2-[(3R,5S)-3,5- |
| | dimethylpiperazin-1-yl]pyrimidin-5-yl}-2,3,4,9-tetrahydro-1H-pyrido[3,4-b]indole; |
| 511 | (1S)-2-[4,6-bis(trifluoromethyl)pyrimidin-2-yl]-6-chloro-1-{2-[(3R)-3- |
| | methylpiperazin-1-yl]pyrimidin-5-yl}-2,3,4,9-tetrahydro-1H-pyrido[3,4-b]indole; |
| 512 | (1S)-2-[4,6-bis(trifluoromethyl)pyrimidin-2-yl]-6-chloro-1-[2-(4,7- |
| | diazaspiro[2.5]octan-7-yl)pyrimidin-5-yl]-2,3,4,9-tetrahydro-1H-pyrido[3,4-b]indole; |
| 514 | (1S)-2-[4,6-bis(trifluoromethyl)pyrimidin-2-yl]-1-[2-(morpholin-4-yl)pyrimidin-5-yl]- |
| | 6-(2H-1,2,3-triazol-2-yl)-2,3,4,9-tetrahydro-1H-pyrido[3,4-b]indole; |
| 517 | (1S)-2-[4,6-bis(trifluoromethyl)pyrimidin-2-yl]-6-chloro-1-{2-[(3S)-3- |
| | methylpiperazin-1-yl]pyrimidin-5-yl}-2,3,4,9-tetrahydro-1H-pyrido[3,4-b]indole; |
| 518 | (1S)-2-[4,6-bis(trifluoromethyl)pyrimidin-2-yl]-6-bromo-1-(2,3-difluoro-4- |
| | methylphenyl)-2,3,4,9-tetrahydro-1H-pyrido[3,4-b]indole; |
| 519 | (1S)-2-[4,6-bis(trifluoromethyl)pyrimidin-2-yl]-1-(2,3-difluoro-4-methylphenyl)-6- |
| | (2H-1,2,3-triazol-2-yl)-2,3,4,9-tetrahydro-1H-pyrido[3,4-b]indole; |
| 520 | (1S)-2-[4,6-bis(trifluoromethyl)pyrimidin-2-yl]-1-(2,3-difluoro-4-methylphenyl)-6-(4- |
| | methyl-2H-1,2,3-triazol-2-yl)-2,3,4,9-tetrahydro-1H-pyrido[3,4-b]indole; and, |
| 528 | (1S)-2-[4,6-bis(trifluoromethyl)pyrimidin-2-yl]-1-(2,3-difluoro-4-methylphenyl)-6- |
| | methoxy-2,3,4,9-tetrahydro-1H-pyrido[3,4-b]indole; |

wherein the form of the compound is selected from the group consisting of a salt, hydrate, solvate, racemate, enantiomer, diastereomer, stereoisomer, and tautomer form thereof.

Another aspect of the compound of Formula (I) or a form thereof includes a compound (Cpd) salt having dihydroorotate dehydrogenase inhibition activity ≤10.0 nM selected from the group consisting of:

| Cpd | Name |
|---|---|
| 357 | 2-[4,6-bis(trifluoromethyl)pyrimidin-2-yl]-6-chloro-1-[6-(morpholin-4-yl)pyridin-3-yl]- |
| | 2,3,4,9-tetrahydro-1H-pyrido[3,4-b]indole formate (1:1); and, |
| 358 | 2-[4,6-bis(trifluoromethyl)pyrimidin-2-yl]-6-chloro-1-[2-(morpholin-4-yl)pyrimidin-5- |
| | yl]-2,3,4,9-tetrahydro-1H-pyrido[3,4-b]indole formate (1:1); |

wherein the form of the compound is selected from the group consisting of a hydrate, solvate, racemate, enantiomer, diastereomer, stereoisomer, and tautomer form thereof.

One aspect of the compound of Formula (I) or a form thereof includes a compound (Cpd) having dihydroorotate dehydrogenase inhibition activity ≤1.0 nM selected from the group consisting of (wherein ¹Cpd #indicates the compound was isolated as a salt form):

| Cpd | Name |
|---|---|
| 7 | 6-chloro-2-(4,6-dichloropyrimidin-2-yl)-1-(4-methylphenyl)-2,3,4,9-tetrahydro-1H- |
| | pyrido[3,4-b]indole; |
| 118 | 6-chloro-1-(2,3-difluoro-4-methylphenyl)-5-fluoro-2-(5-fluoro-4,6- |
| | dimethylpyrimidin-2-yl)-2,3,4,9-tetrahydro-1H-pyrido[3,4-b]indole; |
| 128 | 6-chloro-1-(2,3-difluoro-4-methylphenyl)-2-[4-(trifluoromethyl)pyrimidin-2-yl]- |
| | 2,3,4,9-tetrahydro-1H-pyrido[3,4-b]indole; |
| 140 | 6-chloro-1-(2,3-difluoro-4-methylphenyl)-7-fluoro-2-[4-(trifluoromethyl)pyrimidin- |
| | 2-yl]-2,3,4,9-tetrahydro-1H-pyrido[3,4-b]indole; |
| 144 | 6-chloro-1-(4-chloro-2,3-difluorophenyl)-2-[4-(trifluoromethyl)pyrimidin-2-yl]- |
| | 2,3,4,9-tetrahydro-1H-pyrido[3,4-b]indole; |
| 148 | 6-chloro-1-(2,3-difluoro-4-methylphenyl)-7-fluoro-2-[4-methyl-6- |
| | (trifluoromethyl)pyrimidin-2-yl]-2,3,4,9-tetrahydro-1H-pyrido[3,4-b]indole; |
| 154 | 2-[4,6-bis(trifluoromethyl)pyrimidin-2-yl]-6-chloro-7-fluoro-1-(4-methylphenyl)- |
| | 2,3,4,9-tetrahydro-1H-pyrido[3,4-b]indole; |
| 155 | 2-[4,6-bis(trifluoromethyl)pyrimidin-2-yl]-6-chloro-1-(2,3-difluoro-4- |
| | methylphenyl)-7-fluoro-2,3,4,9-tetrahydro-1H-pyrido[3,4-b]indole; |
| 157 | 2-[4,6-bis(trifluoromethyl)pyrimidin-2-yl]-6-chloro-1-(2,3-difluorophenyl)-7-fluoro- |
| | 2,3,4,9-tetrahydro-1H-pyrido[3,4-b]indole; |
| 188 | 4-{2-[4,6-bis(trifluoromethyl)pyrimidin-2-yl]-6-chloro-7-fluoro-2,3,4,9-tetrahydro- |
| | 1H-pyrido[3,4-b]indol-1-yl}benzonitrile; |
| 189 | (1S)-6-chloro-1-(2,3-difluoro-4-methylphenyl)-5-fluoro-2-(5-fluoro-4,6- |
| | dimethylpyrimidin-2-yl)-2,3,4,9-tetrahydro-1H-pyrido[3,4-b]indole; |
| 195 | 6-chloro-2-(5-chloropyrimidin-2-yl)-1-(2,3-difluoro-4-methylphenyl)-5-fluoro- |
| | 2,3,4,9-tetrahydro-1H-pyrido[3,4-b]indole; |
| 205 | 6-chloro-1-(2,3-difluoro-4-methylphenyl)-2-(4,6-dimethylpyrimidin-2-yl)-5-fluoro- |
| | 2,3,4,9-tetrahydro-1H-pyrido[3,4-b]indole; |
| 206 | 6-chloro-1-(2,3-difluoro-4-methylphenyl)-5-fluoro-2-[4-methyl-6- |
| | (trifluoromethyl)pyrimidin-2-yl]-2,3,4,9-tetrahydro-1H-pyrido[3,4-b]indole; |
| 209 | 2-[4,6-bis(trifluoromethyl)pyrimidin-2-yl]-6-chloro-1-(2,3-difluoro-4- |
| | methylphenyl)-5-fluoro-2,3,4,9-tetrahydro-1H-pyrido[3,4-b]indole; |
| 214 | 2-[6-chloro-1-(2,3-difluoro-4-methylphenyl)-5-fluoro-1,3,4,9-tetrahydro-2H- |
| | pyrido[3,4-b]indol-2-yl]-5-fluoropyrimidine-4-carbonitrile; |
| 232 | 2-[6-chloro-1-(2,3-difluoro-4-methylphenyl)-5-fluoro-1,3,4,9-tetrahydro-2H- |
| | pyrido[3,4-b]indol-2-yl]-6-methylpyrimidine-4-carbonitrile; |
| 233 | (1S)-6-chloro-1-(2,3-difluoro-4-methylphenyl)-2-[4-(trifluoromethyl)pyrimidin-2- |
| | yl]-2,3,4,9-tetrahydro-1H-pyrido[3,4-b]indole; |
| 234 | (1S)-6-chloro-1-(2,3-difluoro-4-methylphenyl)-2-(4-methylpyrimidin-2-yl)-2,3,4,9- |
| | tetrahydro-1H-pyrido[3,4-b]indole; |
| 238 | (1S)-6-chloro-1-(2,3-difluoro-4-methylphenyl)-2-(5-fluoropyrimidin-2-yl)-2,3,4,9- |
| | tetrahydro-1H-pyrido[3,4-b]indole;[[,]] |
| 248 | 6-chloro-1-(2,3-difluoro-4-methylphenyl)-2-[4-(trifluoromethyl)pyrimidin-2-yl]- |
| | 2,3,4,9-tetrahydro-1H-pyrido[3,4-b]indol-8-amine; |
| 259 | 6-chloro-1-(2-fluoro-3-methylphenyl)-2-[4-(trifluoromethyl)pyrimidin-2-yl]-2,3,4,9- |
| | tetrahydro-1H-pyrido[3,4-b]indole; |
| 268 | 1-(4-bromo-2-fluorophenyl)-6-chloro-2-[4-(trifluoromethyl)pyrimidin-2-yl]-2,3,4,9- |
| | tetrahydro-1H-pyrido[3,4-b]indole; |
| 270 | 1-(4-bromo-2,3-difluorophenyl)-6-chloro-2-[4-(trifluoromethyl)pyrimidin-2-yl]- |
| | 2,3,4,9-tetrahydro-1H-pyrido[3,4-b]indole; |
| 276 | 1-(2,3-difluoro-4-methylphenyl)-6-methyl-2-[4-(trifluoromethyl)pyrimidin-2-yl]- |
| | 2,3,4,9-tetrahydro-1H-pyrido[3,4-b]indol-8-ol; |
| 305 | 1-(2,3-difluoro-4-methylphenyl)-6-nitro-2-[4-(trifluoromethyl)pyrimidin-2-yl]- |
| | 2,3,4,9-tetrahydro-1H-pyrido[3,4-b]indole; |
| 340 | 2-[4,6-bis(trifluoromethyl)pyrimidin-2-yl]-6-chloro-1-(2,3-difluoro-4- |
| | methylphenyl)-2,3,4,9-tetrahydro-1H-pyrido[3,4-b]indole; |
| 342 | 6-chloro-1-(2,3-difluoro-4-methylphenyl)-2-(4,6-dimethylpyrimidin-2-yl)-2,3,4,9- |
| | tetrahydro-1H-pyrido[3,4-b]indole; |
| ¹357 | 2-[4,6-bis(trifluoromethyl)pyrimidin-2-yl]-6-chloro-1-[6-(morpholin-4-yl)pyridin-3- |
| | yl]-2,3,4,9-tetrahydro-1H-pyrido[3,4-b]indole; |
| ¹358 | 2-[4,6-bis(trifluoromethyl)pyrimidin-2-yl]-6-chloro-1-[2-(morpholin-4- |
| | yl)pyrimidin-5-yl]-2,3,4,9-tetrahydro-1H-pyrido[3,4-b]indole; |
| 360 | 2-[4,6-bis(trifluoromethyl)pyrimidin-2-yl]-6-chloro-1-(4-methylphenyl)-2,3,4,9- |
| | tetrahydro-1H-pyrido[3,4-b]indole; |
| 381 | 2-[4,6-bis(trifluoromethyl)pyrimidin-2-yl]-6-chloro-1-[2-(4-methylpiperazin-1- |
| | yl)pyrimidin-5-yl]-2,3,4,9-tetrahydro-1H-pyrido[3,4-b]indole; |
| 403 | 6-chloro-1-(2,3-difluoro-4-methylphenyl)-2-[5-(trifluoromethyl)-1,2,4-triazin-3-yl]- |
| | 2,3,4,9-tetrahydro-1H-pyrido[3,4-b]indole; |
| 407 | (1S)-2-[4,6-bis(trifluoromethyl)pyrimidin-2-yl]-6-chloro-1-(2,3-difluoro-4- |
| | methylphenyl)-2,3,4,9-tetrahydro-1H-pyrido[3,4-b]indole; |
| 409 | (1S)-2-[4,6-bis(trifluoromethyl)pyrimidin-2-yl]-6-chloro-1-[6-(morpholin-4- |
| | yl)pyridin-3-yl]-2,3,4,9-tetrahydro-1H-pyrido[3,4-b]indole; |
| 410 | (1S)-2-[4,6-bis(trifluoromethyl)pyrimidin-2-yl]-6-chloro-1-[2-(morpholin-4- |
| | yl)pyrimidin-5-yl]-2,3,4,9-tetrahydro-1H-pyrido[3,4-b]indole; |
| 412 | (1S)-6-chloro-1-(2,3-difluoro-4-methylphenyl)-2-(5-fluoro-4,6-dimethylpyrimidin- |
| | 2-yl)-2,3,4,9-tetrahydro-1H-pyrido[3,4-b]indole; |
| 413 | 2-[(1S)-6-chloro-1-(2,3-difluoro-4-methylphenyl)-1,3,4,9-tetrahydro-2H-pyrido[3,4- |
| | b]indol-2-yl]-5-fluoro-6-methylpyrimidine-4-carbonitrile; |
| 419 | (1S)-2-[4,6-bis(trifluoromethyl)pyrimidin-2-yl]-6-chloro-1-[2-(4-methylpiperazin-1- |
| | yl)pyrimidin-5-yl]-2,3,4,9-tetrahydro-1H-pyrido[3,4-b]indole; |
| 454 | 2-[4,6-bis(trifluoromethyl)pyrimidin-2-yl]-1-[6-(morpholin-4-yl)pyridin-3-yl]-6- |
| | (2H-1,2,3-triazol-2-yl)-2,3,4,9-tetrahydro-1H-pyrido[3,4-b]indole; |
| 473 | (1S)-6-chloro-1-[6-(morpholin-4-yl)pyridin-3-yl]-2-[4-(trifluoromethyl)pyrimidin-2- |
| | yl]-2,3,4,9-tetrahydro-1H-pyrido[3,4-b]indole; |
| 481 | (1S)-2-[4,6-bis(trifluoromethyl)pyrimidin-2-yl]-6-chloro-1-[6-(4-methylpiperazin-1- |
| | yl)pyridin-3-yl]-2,3,4,9-tetrahydro-1H-pyrido[3,4-b]indole; |
| 482 | 2-[4,6-bis(trifluoromethyl)pyrimidin-2-yl]-1-[2-(morpholin-4-yl)pyrimidin-5-yl]-6- |
| | (2H-1,2,3-triazol-2-yl)-2,3,4,9-tetrahydro-1H-pyrido[3,4-b]indole; |
| 485 | (1S)-2-[4,6-bis(trifluoromethyl)pyrimidin-2-yl]-1-[6-(morpholin-4-yl)pyridin-3-yl]- |
| | 6-(2H-1,2,3-triazol-2-yl)-2,3,4,9-tetrahydro-1H-pyrido[3,4-b]indole; |
| 487 | (1S)-2-[4,6-bis(trifluoromethyl)pyrimidin-2-yl]-6-bromo-1-[6-(morpholin-4- |
| | yl)pyridin-3-yl]-2,3,4,9-tetrahydro-1H-pyrido[3,4-b]indole; |
| 499 | (1S)-2-[4,6-bis(trifluoromethyl)pyrimidin-2-yl]-6-bromo-1-[2-(4-methylpiperazin-1- |
| | yl)pyrimidin-5-yl]-2,3,4,9-tetrahydro-1H-pyrido[3,4-b]indole; |
| 500 | (1S)-2-[4,6-bis(trifluoromethyl)pyrimidin-2-yl]-1-[2-(4-methylpiperazin-1- |
| | yl)pyrimidin-5-yl]-6-(2H-1,2,3-triazol-2-yl)-2,3,4,9-tetrahydro-1H-pyrido[3,4- |
| | b]indole; |
| 514 | (1S)-2-[4,6-bis(trifluoromethyl)pyrimidin-2-yl]-1-[2-(morpholin-4-yl)pyrimidin-5- |
| | yl]-6-(2H-1,2,3-triazol-2-yl)-2,3,4,9-tetrahydro-1H-pyrido[3,4-b]indole; |
| 518 | (1S)-2-[4,6-bis(trifluoromethyl)pyrimidin-2-yl]-6-bromo-1-(2,3-difluoro-4- |
| | methylphenyl)-2,3,4,9-tetrahydro-1H-pyrido[3,4-b]indole; and, |
| 519 | (1S)-2-[4,6-bis(trifluoromethyl)pyrimidin-2-yl]-1-(2,3-difluoro-4-methylphenyl)-6- |
| | (2H-1,2,3-triazol-2-yl)-2,3,4,9-tetrahydro-1H-pyrido[3,4-b]indole; |

wherein the form of the compound is selected from the group consisting of a salt, hydrate, solvate, racemate, enantiomer, diastereomer, stereoisomer, and tautomer form thereof.

Another aspect of the compound of Formula (I) or a form thereof includes a compound (Cpd) salt having dihydroorotate dehydrogenase inhibition activity ≤10.0 nM selected from the group consisting of:

| Cpd | Name |
|---|---|
| 357 | 2-[4,6-bis(trifluoromethyl)pyrimidin-2-yl]-6-chloro-1-[6-(morpholin-4-yl)pyridin-3-yl]- |
| | 2,3,4,9-tetrahydro-1H-pyrido[3,4-b]indole formate (1:1); and, |
| 358 | 2-[4,6-bis(trifluoromethyl)pyrimidin-2-yl]-6-chloro-1-[2-(morpholin-4-yl)pyrimidin-5- |
| | yl]-2,3,4,9-tetrahydro-1H-pyrido[3,4-b]indole formate (1:1); |

wherein the form of the compound is selected from the group consisting of a hydrate, solvate, racemate, enantiomer, diastereomer, stereoisomer, and tautomer form thereof.

One aspect of the compound of Formula (I) or a form thereof includes a compound (Cpd) selected from the group consisting of (wherein ¹Cpd #indicates the compound was isolated as a salt form):

| Cpd | Name |
|---|---|
| 253 | 6-chloro-1-(2,3-difluoro-4-methylphenyl)-2-(4,6-dimethyl-1,3,5-triazin-2-yl)-2,3,4,9- |
| | tetrahydro-1H-pyrido[3,4-b]indole; |
| 283 | 1-(4-bromo-2,3-difluorophenyl)-6-chloro-2-(4,6-dimethyl-1,3,5-triazin-2-yl)-2,3,4,9- |
| | tetrahydro-1H-pyrido[3,4-b]indole; |
| 284 | 6-chloro-1-[2,3-difluoro-4-(morpholin-4-yl)phenyl]-2-(4,6-dimethyl-1,3,5-triazin-2- |
| | yl)-2,3,4,9-tetrahydro-1H-pyrido[3,4-b]indole; |
| 289 | 6-chloro-2-(4-chloro-6-methyl-1,3,5-triazin-2-yl)-1-[2-(morpholin-4-yl)pyrimidin-5- |
| | yl]-2,3,4,9-tetrahydro-1H-pyrido[3,4-b]indole; |
| 290 | 4-{6-chloro-1-[2-(morpholin-4-yl)pyrimidin-5-yl]-1,3,4,9-tetrahydro-2H-pyrido[3,4- |
| | b]indol-2-yl}-N,N,6-trimethyl-1,3,5-triazin-2-amine; |
| 292 | 6-chloro-2-(4,6-dimethyl-1,3,5-triazin-2-yl)-1-(4-methylphenyl)-2,3,4,9-tetrahydro- |
| | 1H-pyrido[3,4-b]indol-8-amine; |
| 293 | 6-chloro-2-(4,6-dimethyl-1,3,5-triazin-2-yl)-1-[2-(morpholin-4-yl)pyrimidin-5-yl]- |
| | 2,3,4,9-tetrahydro-1H-pyrido[3,4-b]indole; |
| 294 | 6-chloro-2-(4-methyl-1,3,5-triazin-2-yl)-1-[2-(morpholin-4-yl)pyrimidin-5-yl]-2,3,4,9- |
| | tetrahydro-1H-pyrido[3,4-b]indole; |
| 295 | 6-chloro-2-(4-methoxy-6-methyl-1,3,5-triazin-2-yl)-1-[2-(morpholin-4-yl)pyrimidin- |
| | 5-yl]-2,3,4,9-tetrahydro-1H-pyrido[3,4-b]indole; |
| 296 | 6-chloro-2-[4-methyl-6-(morpholin-4-yl)-1,3,5-triazin-2-yl]-1-[2-(morpholin-4- |
| | yl)pyrimidin-5-yl]-2,3,4,9-tetrahydro-1H-pyrido[3,4-b]indole; |
| 297 | 4-{6-chloro-1-[2-(morpholin-4-yl)pyrimidin-5-yl]-1,3,4,9-tetrahydro-2H-pyrido[3,4- |
| | b]indol-2-yl}-N,N-dimethyl-1,3,5-triazin-2-amine; |
| 299 | 6-bromo-2-(4,6-dimethyl-1,3,5-triazin-2-yl)-1-[2-(morpholin-4-yl)pyrimidin-5-yl]- |
| | 2,3,4,9-tetrahydro-1H-pyrido[3,4-b]indole; |
| 300 | 2-(4,6-dimethyl-1,3,5-triazin-2-yl)-1-[2-(morpholin-4-yl)pyrimidin-5-yl]-6-(2H-1,2,3- |
| | triazol-2-yl)-2,3,4,9-tetrahydro-1H-pyrido[3,4-b]indole; |
| 304 | 6-chloro-1-(2,3-difluoro-4-methylphenyl)-2-(4,6-dimethyl-1,3,5-triazin-2-yl)-2,3,4,9- |
| | tetrahydro-1H-pyrido[3,4-b]indol-8-amine; |
| 309 | 6-chloro-2-(4,6-dimethyl-1,3,5-triazin-2-yl)-1-[2-(morpholin-4-yl)pyrimidin-5-yl]- |
| | 2,3,4,9-tetrahydro-1H-pyrido[3,4-b]indol-8-amine; |
| 313 | 2-(4,6-dimethyl-1,3,5-triazin-2-yl)-1-[2-(morpholin-4-yl)pyrimidin-5-yl]-6-nitro- |
| | 2,3,4,9-tetrahydro-1H-pyrido[3,4-b]indole; |
| 315 | N,N-dibutyl-4-{6-chloro-1-[2-(morpholin-4-yl)pyrimidin-5-yl]-1,3,4,9-tetrahydro-2H- |
| | pyrido[3,4-b]indol-2-yl}-6-methyl-1,3,5-triazin-2-amine; |
| 317 | N-butyl-4-{6-chloro-1-[2-(morpholin-4-yl)pyrimidin-5-yl]-1,3,4,9-tetrahydro-2H- |
| | pyrido[3,4-b]indol-2-yl}-N,6-dimethyl-1,3,5-triazin-2-amine; |
| 318 | 2-(4-butoxy-6-methyl-1,3,5-triazin-2-yl)-6-chloro-1-[2-(morpholin-4-yl)pyrimidin-5- |
| | yl]-2,3,4,9-tetrahydro-1H-pyrido[3,4-b]indole; |
| 320 | 6-chloro-2-(4,6-dimethyl-1,3,5-triazin-2-yl)-1-[2-(morpholin-4-yl)pyrimidin-5-yl]- |
| | 2,3,4,9-tetrahydro-1H-pyrido[3,4-b]indol-7-amine; |
| 321 | 6-chloro-2-(4,6-dimethyl-1,3,5-triazin-2-yl)-1-[6-(morpholin-4-yl)pyridin-3-yl]- |
| | 2,3,4,9-tetrahydro-1H-pyrido[3,4-b]indol-8-amine; |
| 322 | 6-chloro-1-(4-methylphenyl)-2-(4-methyl-1,3,5-triazin-2-yl)-2,3,4,9-tetrahydro-1H- |
| | pyrido[3,4-b]indole; |
| 324 | 6-chloro-2-(4,6-dimethyl-1,3,5-triazin-2-yl)-1-[6-(morpholin-4-yl)pyridin-3-yl]- |
| | 2,3,4,9-tetrahydro-1H-pyrido[3,4-b]indole; |
| 325 | 6-chloro-2-(4,6-dimethyl-1,3,5-triazin-2-yl)-1-[2-(morpholin-4-yl)pyrimidin-5-yl]-5- |
| | nitro-2,3,4,9-tetrahydro-1H-pyrido[3,4-b]indole; |
| 327 | 6-chloro-2-(4,6-dimethyl-1,3,5-triazin-2-yl)-1-[2-(morpholin-4-yl)pyrimidin-5-yl]- |
| | 2,3,4,9-tetrahydro-1H-pyrido[3,4-b]indol-5-amine; |
| 331 | 2-(4,6-dimethyl-1,3,5-triazin-2-yl)-N,N-dimethyl-1-[2-(morpholin-4-yl)pyrimidin-5- |
| | yl]-2,3,4,9-tetrahydro-1H-pyrido[3,4-b]indol-6-amine; |
| 333 | 2-(4,6-dimethyl-1,3,5-triazin-2-yl)-1-[2-(morpholin-4-yl)pyrimidin-5-yl]-6-nitro- |
| | 2,3,4,9-tetrahydro-1H-pyrido[3,4-b]indol-8-ol; |
| 336 | 6-chloro-2-(4,6-dimethyl-1,3,5-triazin-2-yl)-1-[2-(2-oxa-5-azabicyclo[2.2.1]heptan-5- |
| | yl)pyrimidin-5-yl]-2,3,4,9-tetrahydro-1H-pyrido[3,4-b]indole; |
| 338 | 6-chloro-2-(4,6-dimethyl-1,3,5-triazin-2-yl)-1-[2-(3-oxa-8-azabicyclo[3.2.1]octan-8- |
| | yl)pyrimidin-5-yl]-2,3,4,9-tetrahydro-1H-pyrido[3,4-b]indole; |
| 339 | 6-chloro-2-(4,6-dimethyl-1,3,5-triazin-2-yl)-1-[6-(morpholin-4-yl)pyridin-3-yl]- |
| | 2,3,4,9-tetrahydro-1H-pyrido[3,4-b]indol-8-ol; |
| 345 | 1-[2-(azetidin-1-yl)pyrimidin-5-yl]-6-chloro-2-(4,6-dimethyl-1,3,5-triazin-2-yl)- |
| | 2,3,4,9-tetrahydro-1H-pyrido[3,4-b]indole; |
| 346 | 6-chloro-2-(4,6-dimethyl-1,3,5-triazin-2-yl)-1-[2-(methylsulfanyl)pyrimidin-5-yl]- |
| | 2,3,4,9-tetrahydro-1H-pyrido[3,4-b]indole; |
| 347 | (1S)-6-chloro-1-(2,3-difluoro-4-methylphenyl)-2-(4,6-dimethyl-1,3,5-triazin-2-yl)- |
| | 2,3,4,9-tetrahydro-1H-pyrido[3,4-b]indole; |
| 348 | 6-chloro-1-(2,3-difluoro-4-methylphenyl)-2-(4,6-dimethyl-1,3,5-triazin-2-yl)-2,3,4,9- |
| | tetrahydro-1H-pyrido[3,4-b]indol-8-ol; |
| 355 | 6-chloro-2-(4,6-dimethyl-1,3,5-triazin-2-yl)-1-(4-methylphenyl)-2,3,4,9-tetrahydro- |
| | 1H-pyrido[3,4-b]indole; |
| 370 | 6-chloro-2-(4,6-dimethyl-1,3,5-triazin-2-yl)-1-[2-(morpholin-4-yl)pyrimidin-5-yl]- |
| | 2,3,4,9-tetrahydro-1H-pyrido[3,4-b]indol-8-ol; |
| 376 | 6-chloro-2-(4,6-dimethyl-1,3,5-triazin-2-yl)-1-{6-[(1R,4R)-2-oxa-5- |
| | azabicyclo[2.2.1]heptan-5-yl]pyridin-3-yl}-2,3,4,9-tetrahydro-1H-pyrido[3,4-b]indole; |
| 377 | 6-chloro-2-(4,6-dimethyl-1,3,5-triazin-2-yl)-1-{6-[(1S,4S)-2-oxa-5- |
| | azabicyclo[2.2.1]heptan-5-yl]pyridin-3-yl}-2,3,4,9-tetrahydro-1H-pyrido[3,4-b]indole; |
| 380 | 6-chloro-2-(4,6-dimethyl-1,3,5-triazin-2-yl)-1-[2-(4-methylpiperazin-1-yl)pyrimidin- |
| | 5-yl]-2,3,4,9-tetrahydro-1H-pyrido[3,4-b]indole; |
| 386 | (1S)-2-[4,6-bis(trifluoromethyl)-1,3,5-triazin-2-yl]-6-chloro-1-(4-methylphenyl)- |
| | 2,3,4,9-tetrahydro-1H-pyrido[3,4-b]indole; |
| 387 | (1S)-2-[4,6-bis(trifluoromethyl)-1,3,5-triazin-2-yl]-6-chloro-1-(2,3-difluoro-4- |
| | methylphenyl)-2,3,4,9-tetrahydro-1H-pyrido[3,4-b]indole; |
| 388 | 2-[4,6-bis(trifluoromethyl)-1,3,5-triazin-2-yl]-6-chloro-1-[2-(morpholin-4- |
| | yl)pyrimidin-5-yl]-2,3,4,9-tetrahydro-1H-pyrido[3,4-b]indole; |
| 389 | (1S)-6-chloro-1-(2,3-difluoro-4-methylphenyl)-2-[4-(methylsulfanyl)-6- |
| | (trifluoromethyl)-1,3,5-triazin-2-yl]-2,3,4,9-tetrahydro-1H-pyrido[3,4-b]indole; |
| 390 | 4-[(1S)-6-chloro-1-(2,3-difluoro-4-methylphenyl)-1,3,4,9-tetrahydro-2H-pyrido[3,4- |
| | b]indol-2-yl]-6-(trifluoromethyl)-1,3,5-triazine-2-carbonitrile; |
| 399 | 4-[(1S)-6-chloro-1-(4-methylphenyl)-1,3,4,9-tetrahydro-2H-pyrido[3,4-b]indol-2-yl]- |
| | 1,3,5-triazine-2-carbonitrile; |
| 402 | (1S)-6-chloro-2-(4,6-dimethyl-1,3,5-triazin-2-yl)-1-(4-methylphenyl)-2,3,4,9- |
| | tetrahydro-1H-pyrido[3,4-b]indole; |
| 404 | 2-[4,6-bis(trifluoromethyl)-1,3,5-triazin-2-yl]-6-chloro-1-[6-(morpholin-4-yl)pyridin- |
| | 3-yl]-2,3,4,9-tetrahydro-1H-pyrido[3,4-b]indole; |
| 405 | (1S)-2-[4,6-bis(trifluoromethyl)-1,3,5-triazin-2-yl]-6-chloro-1-[6-(morpholin-4- |
| | yl)pyridin-3-yl]-2,3,4,9-tetrahydro-1H-pyrido[3,4-b]indole; |
| 406 | (1S)-2-[4,6-bis(trifluoromethyl)-1,3,5-triazin-2-yl]-6-chloro-1-[2-(morpholin-4- |
| | yl)pyrimidin-5-yl]-2,3,4,9-tetrahydro-1H-pyrido[3,4-b]indole; |
| 411 | 4-[(1S)-6-chloro-1-(4-methylphenyl)-1,3,4,9-tetrahydro-2H-pyrido[3,4-b]indol-2-yl]- |
| | 6-methyl-1,3,5-triazine-2-carbonitrile; |
| 414 | 4-{(1S)-6-chloro-1-[2-(morpholin-4-yl)pyrimidin-5-yl]-1,3,4,9-tetrahydro-2H- |
| | pyrido[3,4-b]indol-2-yl}-1,3,5-triazine-2-carbonitrile; |
| 415 | 4-{(1S)-6-chloro-1-[2-(morpholin-4-yl)pyrimidin-5-yl]-1,3,4,9-tetrahydro-2H- |
| | pyrido[3,4-b]indol-2-yl}-6-methyl-1,3,5-triazine-2-carbonitrile; |
| 418 | (1S)-6-chloro-1-(4-methylphenyl)-2-[4-(trifluoromethyl)-1,3,5-triazin-2-yl]-2,3,4,9- |
| | tetrahydro-1H-pyrido[3,4-b]indole; |
| 420 | (1S)-2-[4,6-bis(trifluoromethyl)-1,3,5-triazin-2-yl]-6-chloro-1-[2-(4-methylpiperazin- |
| | 1-yl)pyrimidin-5-yl]-2,3,4,9-tetrahydro-1H-pyrido[3,4-b]indole; |
| 426 | 4-[(1S)-6-chloro-1-(4-methylphenyl)-1,3,4,9-tetrahydro-2H-pyrido[3,4-b]indol-2-yl]- |
| | 6-(trifluoromethyl)-1,3,5-triazin-2-amine; |
| 427 | 4-[(1S)-6-chloro-1-(2,3-difluoro-4-methylphenyl)-1,3,4,9-tetrahydro-2H-pyrido[3,4- |
| | b]indol-2-yl]-6-(trifluoromethyl)-1,3,5-triazin-2-amine; |
| 428 | 6-chloro-1-[6-(morpholin-4-yl)pyridin-3-yl]-2-[4-(morpholin-4-yl)-6- |
| | (trifluoromethyl)-1,3,5-triazin-2-yl]-2,3,4,9-tetrahydro-1H-pyrido[3,4-b]indole; |
| 435 | 4-[(1S)-6-chloro-1-(2,3-difluoro-4-methylphenyl)-1,3,4,9-tetrahydro-2H-pyrido[3,4- |
| | b]indol-2-yl]-N,N-dimethyl-6-(trifluoromethyl)-1,3,5-triazin-2-amine; |
| 436 | (1S)-6-chloro-1-(2,3-difluoro-4-methylphenyl)-2-[4-(morpholin-4-yl)-6- |
| | (trifluoromethyl)-1,3,5-triazin-2-yl]-2,3,4,9-tetrahydro-1H-pyrido[3,4-b]indole; |
| 437 | 6-chloro-1-(4-methylphenyl)-2-[4-methyl-6-(trifluoromethyl)-1,3,5-triazin-2-yl]- |
| | 2,3,4,9-tetrahydro-1H-pyrido[3,4-b]indole; |
| 438 | 6-chloro-1-(2,3-difluoro-4-methylphenyl)-2-[4-methyl-6-(trifluoromethyl)-1,3,5- |
| | triazin-2-yl]-2,3,4,9-tetrahydro-1H-pyrido[3,4-b]indole; |
| 439 | 6-chloro-2-[4-methyl-6-(trifluoromethyl)-1,3,5-triazin-2-yl]-1-[6-(morpholin-4- |
| | yl)pyridin-3-yl]-2,3,4,9-tetrahydro-1H-pyrido[3,4-b]indole; |
| 440 | 6-chloro-2-[4-methyl-6-(trifluoromethyl)-1,3,5-triazin-2-yl]-1-[2-(morpholin-4- |
| | yl)pyrimidin-5-yl]-2,3,4,9-tetrahydro-1H-pyrido[3,4-b]indole; |
| 441 | 4-[(1S)-6-chloro-1-(4-methylphenyl)-1,3,4,9-tetrahydro-2H-pyrido[3,4-b]indol-2-yl]- |
| | N,N-dimethyl-6-(trifluoromethyl)-1,3,5-triazin-2-amine; |
| 442 | (1S)-6-chloro-1-(4-methylphenyl)-2-[4-(morpholin-4-yl)-6-(trifluoromethyl)-1,3,5- |
| | triazin-2-yl]-2,3,4,9-tetrahydro-1H-pyrido[3,4-b]indole; |
| 443 | 4-{(1S)-6-chloro-1-[6-(morpholin-4-yl)pyridin-3-yl]-1,3,4,9-tetrahydro-2H- |
| | pyrido[3,4-b]indol-2-yl}-N,N-dimethyl-6-(trifluoromethyl)-1,3,5-triazin-2-amine; |
| 444 | 4-{(1S)-6-chloro-1-[2-(morpholin-4-yl)pyrimidin-5-yl]-1,3,4,9-tetrahydro-2H- |
| | pyrido[3,4-b]indol-2-yl}-N,N-dimethyl-6-(trifluoromethyl)-1,3,5-triazin-2-amine; |
| 445 | (1S)-6-chloro-1-[2-(morpholin-4-yl)pyrimidin-5-yl]-2-[4-(morpholin-4-yl)-6- |
| | (trifluoromethyl)-1,3,5-triazin-2-yl]-2,3,4,9-tetrahydro-1H-pyrido[3,4-b]indole; |
| 446 | (1S)-6-chloro-1-[6-(morpholin-4-yl)pyridin-3-yl]-2-[4-(morpholin-4-yl)-6- |
| | (trifluoromethyl)-1,3,5-triazin-2-yl]-2,3,4,9-tetrahydro-1H-pyrido[3,4-b]indole; |
| 448 | 4-[6-chloro-2-(4,6-dimethyl-1,3,5-triazin-2-yl)-2,3,4,9-tetrahydro-1H-pyrido[3,4- |
| | b]indol-1-yl]benzonitrile; |
| 449 | 6-chloro-1-(4-chlorophenyl)-2-(4,6-dimethyl-1,3,5-triazin-2-yl)-2,3,4,9-tetrahydro- |
| | 1H-pyrido[3,4-b]indole; |
| 450 | 6-chloro-2-(4,6-dimethyl-1,3,5-triazin-2-yl)-1-(4-fluorophenyl)-2,3,4,9-tetrahydro- |
| | 1H-pyrido[3,4-b]indole; |
| 451 | 6-chloro-1-(2,4-difluorophenyl)-2-(4,6-dimethyl-1,3,5-triazin-2-yl)-2,3,4,9-tetrahydro- |
| | 1H-pyrido[3,4-b]indole; |
| 452 | 6-chloro-2-(4,6-dimethyl-1,3,5-triazin-2-yl)-1-(2-fluoro-4-methylphenyl)-2,3,4,9- |
| | tetrahydro-1H-pyrido[3,4-b]indole; |
| 453 | 4-[6-chloro-2-(4,6-dimethyl-1,3,5-triazin-2-yl)-2,3,4,9-tetrahydro-1H-pyrido[3,4- |
| | b]indol-1-yl]-3-fluorobenzonitrile; |
| 457 | (1S)-6-chloro-2-[4-(3,3-difluoroazetidin-1-yl)-6-methyl-1,3,5-triazin-2-yl]-1-[2- |
| | (morpholin-4-yl)pyrimidin-5-yl]-2,3,4,9-tetrahydro-1H-pyrido[3,4-b]indole; |
| 459 | 6-chloro-2-(4,6-dimethyl-1,3,5-triazin-2-yl)-1-[2-fluoro-6-(morpholin-4-yl)pyridin-3- |
| | yl]-2,3,4,9-tetrahydro-1H-pyrido[3,4-b]indole; |
| 460 | (1S)-6-chloro-1-[2-(morpholin-4-yl)pyrimidin-5-yl]-2-[4-(trifluoromethyl)-1,3,5- |
| | triazin-2-yl]-2,3,4,9-tetrahydro-1H-pyrido[3,4-b]indole; |
| 461 | (1S)-6-chloro-1-(2,3-difluoro-4-methylphenyl)-2-[4-(trifluoromethyl)-1,3,5-triazin-2- |
| | yl]-2,3,4,9-tetrahydro-1H-pyrido[3,4-b]indole; |
| 462 | (1S)-6-chloro-1-[6-(morpholin-4-yl)pyridin-3-yl]-2-[4-(trifluoromethyl)-1,3,5-triazin- |
| | 2-yl]-2,3,4,9-tetrahydro-1H-pyrido[3,4-b]indole; |
| 464 | (1S)-6-chloro-1-(2,3-difluoro-4-methylphenyl)-2-[4-methyl-6-(trifluoromethyl)-1,3,5- |
| | triazin-2-yl]-2,3,4,9-tetrahydro-1H-pyrido[3,4-b]indole; |
| 465 | (1S)-6-chloro-2-[4-methyl-6-(trifluoromethyl)-1,3,5-triazin-2-yl]-1-[6-(morpholin-4- |
| | yl)pyridin-3-yl]-2,3,4,9-tetrahydro-1H-pyrido[3,4-b]indole; |
| 466 | (1S)-6-chloro-2-[4-methyl-6-(trifluoromethyl)-1,3,5-triazin-2-yl]-1-[2-(morpholin-4- |
| | yl)pyrimidin-5-yl]-2,3,4,9-tetrahydro-1H-pyrido[3,4-b]indole; |
| 467 | (1S)-6-chloro-1-(4-methylphenyl)-2-[4-methyl-6-(trifluoromethyl)-1,3,5-triazin-2-yl]- |
| | 2,3,4,9-tetrahydro-1H-pyrido[3,4-b]indole; |
| 468 | 6-chloro-1-(4-chloro-2-fluorophenyl)-2-(4,6-dimethyl-1,3,5-triazin-2-yl)-2,3,4,9- |
| | tetrahydro-1H-pyrido[3,4-b]indole; |
| 474 | (1S)-6-chloro-1-[2-(4-methylpiperazin-1-yl)pyrimidin-5-yl]-2-[4-(methylsulfanyl)-6- |
| | (trifluoromethyl)-1,3,5-triazin-2-yl]-2,3,4,9-tetrahydro-1H-pyrido[3,4-b]indole; |
| 476 | (1S)-6-chloro-1-(2,3-difluoro-4-methylphenyl)-2-[4-fluoro-6-(4-methylpiperazin-1- |
| | yl)-1,3,5-triazin-2-yl]-2,3,4,9-tetrahydro-1H-pyrido[3,4-b]indole; |
| 477 | (1S)-6-chloro-1-(2,3-difluoro-4-methylphenyl)-2-[4-(4-methylpiperazin-1-yl)-6- |
| | (trifluoromethyl)-1,3,5-triazin-2-yl]-2,3,4,9-tetrahydro-1H-pyrido[3,4-b]indole; |
| 478 | (1S)-6-chloro-2-[4-(4-methylpiperazin-1-yl)-6-(trifluoromethyl)-1,3,5-triazin-2-yl]-1- |
| | [2-(morpholin-4-yl)pyrimidin-5-yl]-2,3,4,9-tetrahydro-1H-pyrido[3,4-b]indole; |
| 479 | (1S)-6-chloro-1-[2-(4-methylpiperazin-1-yl)pyrimidin-5-yl]-2-[4-(4-methylpiperazin- |
| | 1-yl)-6-(trifluoromethyl)-1,3,5-triazin-2-yl]-2,3,4,9-tetrahydro-1H-pyrido[3,4- |
| | b]indole; |
| 486 | (1S)-2-[4,6-bis(trifluoromethyl)-1,3,5-triazin-2-yl]-1-[6-(morpholin-4-yl)pyridin-3- |
| | yl]-6-(2H-1,2,3-triazol-2-yl)-2,3,4,9-tetrahydro-1H-pyrido[3,4-b]indole; |
| 488 | (1S)-6-chloro-1-[2-(4-methylpiperazin-1-yl)pyrimidin-5-yl]-2-[4-(trifluoromethyl)- |
| | 1,3,5-triazin-2-yl]-2,3,4,9-tetrahydro-1H-pyrido[3,4-b]indole; |
| 489 | (1S)-6-chloro-1-[6-(4-methylpiperazin-1-yl)pyridin-3-yl]-2-[4-(methylsulfanyl)-6- |
| | (trifluoromethyl)-1,3,5-triazin-2-yl]-2,3,4,9-tetrahydro-1H-pyrido[3,4-b]indole; |
| ¹491 | (1S)-6-chloro-1-[6-(4-methylpiperazin-1-yl)pyridin-3-yl]-2-[4-(trifluoromethyl)-1,3,5- |
| | triazin-2-yl]-2,3,4,9-tetrahydro-1H-pyrido[3,4-b]indole; |
| 492 | 2-[4,6-bis(trifluoromethyl)-1,3,5-triazin-2-yl]-1-(2,3-difluoro-4-methylphenyl)-6- |
| | fluoro-2,3,4,9-tetrahydro-1H-pyrido[3,4-b]indole; |
| 494 | 1-(2,3-difluoro-4-methylphenyl)-6-fluoro-2-[4-(trifluoromethyl)-1,3,5-triazin-2-yl]- |
| | 2,3,4,9-tetrahydro-1H-pyrido[3,4-b]indole; |
| 495 | 1-(2,3-difluoro-4-methylphenyl)-6-fluoro-2-[4-(methylsulfanyl)-6-(trifluoromethyl)- |
| | 1,3,5-triazin-2-yl]-2,3,4,9-tetrahydro-1H-pyrido[3,4-b]indole; |
| 497 | 2-[4,6-bis(trifluoromethyl)-1,3,5-triazin-2-yl]-6-chloro-1-(2,3-difluoro-4- |
| | methylphenyl)-2,3,4,9-tetrahydro-1H-pyrido[3,4-b]indole; |
| 498 | (1S)-1-[6-(morpholin-4-yl)pyridin-3-yl]-6-(2H-1,2,3-triazol-2-yl)-2-[4- |
| | (trifluoromethyl)-1,3,5-triazin-2-yl]-2,3,4,9-tetrahydro-1H-pyrido[3,4-b]indole; |
| 501 | (1S)-2-[4,6-bis(trifluoromethyl)-1,3,5-triazin-2-yl]-1-[2-(4-methylpiperazin-1- |
| | yl)pyrimidin-5-yl]-6-(2H-1,2,3-triazol-2-yl)-2,3,4,9-tetrahydro-1H-pyrido[3,4- |
| | b]indole; |
| 504 | methyl 4-[(1S)-6-chloro-1-(2,3-difluoro-4-methylphenyl)-1,3,4,9-tetrahydro-2H- |
| | pyrido[3,4-b]indol-2-yl]-6-(trifluoromethyl)-1,3,5-triazine-2-carboxylate; |
| 505 | (1S)-6-chloro-1-(2,3-difluoro-4-methylphenyl)-2-[4-(trichloromethyl)-6- |
| | (trifluoromethyl)-1,3,5-triazin-2-yl]-2,3,4,9-tetrahydro-1H-pyrido[3,4-b]indole; |
| 506 | (1S)-1-[2-(4-methylpiperazin-1-yl)pyrimidin-5-yl]-6-(2H-1,2,3-triazol-2-yl)-2-[4- |
| | (trifluoromethyl)-1,3,5-triazin-2-yl]-2,3,4,9-tetrahydro-1H-pyrido[3,4-b]indole; |
| 507 | (1S)-6-chloro-2-[4-(difluoromethyl)-6-(trifluoromethyl)-1,3,5-triazin-2-yl]-1-[2- |
| | (morpholin-4-yl)pyrimidin-5-yl]-2,3,4,9-tetrahydro-1H-pyrido[3,4-b]indole; |
| 508 | (1S)-6-chloro-2-[4-(difluoromethyl)-6-(trifluoromethyl)-1,3,5-triazin-2-yl]-1-[2-(4- |
| | methylpiperazin-1-yl)pyrimidin-5-yl]-2,3,4,9-tetrahydro-1H-pyrido[3,4-b]indole; |
| 509 | (1S)-6-chloro-1-[2-(4-methylpiperazin-1-yl)pyrimidin-5-yl]-2-[4-(morpholin-4-yl)-6- |
| | (trifluoromethyl)-1,3,5-triazin-2-yl]-2,3,4,9-tetrahydro-1H-pyrido[3,4-b]indole; |
| 510 | (1S)-6-chloro-1-(2,3-difluoro-4-methylphenyl)-2-[4-(difluoromethyl)-6- |
| | (trifluoromethyl)-1,3,5-triazin-2-yl]-2,3,4,9-tetrahydro-1H-pyrido[3,4-b]indole; |
| 515 | (1S)-1-[2-(morpholin-4-yl)pyrimidin-5-yl]-6-(2H-1,2,3-triazol-2-yl)-2-[4- |
| | (trifluoromethyl)-1,3,5-triazin-2-yl]-2,3,4,9-tetrahydro-1H-pyrido[3,4-b]indole; |
| 516 | (1S)-2-[4,6-bis(trifluoromethyl)-1,3,5-triazin-2-yl]-1-[2-(morpholin-4-yl)pyrimidin-5- |
| | yl]-6-(2H-1,2,3-triazol-2-yl)-2,3,4,9-tetrahydro-1H-pyrido[3,4-b]indole; |
| 521 | {4-[(1S)-6-chloro-1-(2,3-difluoro-4-methylphenyl)-1,3,4,9-tetrahydro-2H-pyrido[3,4- |
| | b]indol-2-yl]-6-(trifluoromethyl)-1,3,5-triazin-2-yl}methanol; |
| 522 | (1S)-2-[4,6-bis(trifluoromethyl)-1,3,5-triazin-2-yl]-6-chloro-1-(2-methoxypyrimidin- |
| | 5-yl)-2,3,4,9-tetrahydro-1H-pyrido[3,4-b]indole; |
| 523 | (1S)-2-[4,6-bis(trifluoromethyl)-1,3,5-triazin-2-yl]-6-bromo-1-(2,3-difluoro-4- |
| | methylphenyl)-2,3,4,9-tetrahydro-1H-pyrido[3,4-b]indole; |
| 524 | (1S)-2-[4,6-bis(trifluoromethyl)-1,3,5-triazin-2-yl]-1-(2,3-difluoro-4-methylphenyl)-6- |
| | (2H-1,2,3-triazol-2-yl)-2,3,4,9-tetrahydro-1H-pyrido[3,4-b]indole; |
| 525 | (1S)-2-[4,6-bis(trifluoromethyl)-1,3,5-triazin-2-yl]-1-(2,3-difluoro-4-methylphenyl)-6- |
| | (4-methyl-2H-1,2,3-triazol-2-yl)-2,3,4,9-tetrahydro-1H-pyrido[3,4-b]indole; |
| 526 | (1S)-6-chloro-1-(2,3-difluoro-4-methylphenyl)-2-[4-(fluoromethyl)-6- |
| | (trifluoromethyl)-1,3,5-triazin-2-yl]-2,3,4,9-tetrahydro-1H-pyrido[3,4-b]indole; |
| 527 | (1S)-2-[4,6-bis(trifluoromethyl)-1,3,5-triazin-2-yl]-1-(2,3-difluoro-4-methylphenyl)-6- |
| | nitro-2,3,4,9-tetrahydro-1H-pyrido[3,4-b]indole; |
| 529 | (1S)-2-[4,6-bis(trifluoromethyl)-1,3,5-triazin-2-yl]-1-(2,3-difluoro-4-methylphenyl)-6- |
| | (4-methyl-1H-pyrazol-1-yl)-2,3,4,9-tetrahydro-1H-pyrido[3,4-b]indole; |
| 530 | (1S)-2-[4,6-bis(trifluoromethyl)-1,3,5-triazin-2-yl]-1-(2,3-difluoro-4-methylphenyl)-6- |
| | (3-methyl-1H-pyrazol-1-yl)-2,3,4,9-tetrahydro-1H-pyrido[3,4-b]indole; |
| 531 | N²-(5-{(1S)-2-[4,6-bis(trifluoromethyl)-1,3,5-triazin-2-yl]-6-chloro-2,3,4,9- |
| | tetrahydro-1H-pyrido[3,4-b]indol-1-yl}pyrimidin-2-yl)-N¹,N¹-dimethylethane-1,2-diamine; |
| 532 | methyl 5-{(1S)-2-[4,6-bis(trifluoromethyl)-1,3,5-triazin-2-yl]-6-chloro-2,3,4,9- |
| | tetrahydro-1H-pyrido[3,4-b]indol-1-yl}pyrimidine-2-carboxylate; |
| 533 | 1-(5-{(1S)-2-[4,6-bis(trifluoromethyl)-1,3,5-triazin-2-yl]-6-chloro-2,3,4,9-tetrahydro- |
| | 1H-pyrido[3,4-b]indol-1-yl}pyrimidin-2-yl)ethan-1-one; |
| 534 | (1S)-2-[4,6-bis(trifluoromethyl)-1,3,5-triazin-2-yl]-6-chloro-1-[2-(2- |
| | methoxyethoxy)pyrimidin-5-yl]-2,3,4,9-tetrahydro-1H-pyrido[3,4-b]indole; |
| 535 | 2-[(5-{(1S)-2-[4,6-bis(trifluoromethyl)-1,3,5-triazin-2-yl]-6-chloro-2,3,4,9-tetrahydro- |
| | 1H-pyrido[3,4-b]indol-1-yl}pyrimidin-2-yl)amino]ethan-1-ol; |
| 536 | 2-[4,6-bis(trifluoromethyl)-1,3,5-triazin-2-yl]-6-chloro-1-[2-(2- |
| | methoxyethoxy)pyrimidin-5-yl]-2,3,4,9-tetrahydro-1H-pyrido[3,4-b]indole; |
| 537 | N-(5-{(1S)-2-[4,6-bis(trifluoromethyl)-1,3,5-triazin-2-yl]-6-chloro-2,3,4,9-tetrahydro- |
| | 1H-pyrido[3,4-b]indol-1-yl}pyrimidin-2-yl)-β-alanine; |
| 538 | 4-[(5-{(1S)-2-[4,6-bis(trifluoromethyl)-1,3,5-triazin-2-yl]-6-chloro-2,3,4,9-tetrahydro- |
| | 1H-pyrido[3,4-b]indol-1-yl}pyrimidin-2-yl)amino]butanoic acid; and |
| 539 | {2-[(5-{(1S)-2-[4,6-bis(trifluoromethyl)-1,3,5-triazin-2-yl]-6-chloro-2,3,4,9- |
| | tetrahydro-1H-pyrido[3,4-b]indol-1-yl}pyrimidin-2-yl)amino]ethoxy}acetic acid; |

wherein the form of the compound is selected from the group consisting of a salt, hydrate, solvate, racemate, enantiomer, diastereomer, stereoisomer, and tautomer form thereof.

Another aspect of the compound of Formula (I) or a form thereof includes a compound (Cpd) salt selected from the group consisting of:

| Cpd | Name |
|---|---|
| 491 | (1S)-6-chloro-1-[6-(4-methylpiperazin-1-yl)pyridin-3-yl]-2-[4-(trifluoromethyl)-1,3,5- |
| | triazin-2-yl]-2,3,4,9-tetrahydro-1H-py.rido[3,4-b]indole formate (1:1); |

wherein the form of the compound is selected from the group consisting of a hydrate, solvate, racemate, enantiomer, diastereomer, stereoisomer, and tautomer form thereof.

One aspect of the compound of Formula (I) or a form thereof includes a compound (Cpd) having dihydroorotate dehydrogenase inhibition activity ≤10.0 nM selected from the group consisting of (wherein ¹Cpd #indicates the compound was isolated as a salt form):

| Cpd | Name |
|---|---|
| 253 | 6-chloro-1-(2,3-difluoro-4-methylphenyl)-2-(4,6-dimethyl-1,3,5-triazin-2-yl)-2,3,4,9- |
| | tetrahydro-1H-pyrido[3,4-b]indole; |
| 283 | 1-(4-bromo-2,3-difluorophenyl)-6-chloro-2-(4,6-dimethyl-1,3,5-triazin-2-yl)-2,3,4,9- |
| | tetrahydro-1H-pyrido[3,4-b]indole; |
| 299 | 6-bromo-2-(4,6-dimethyl-1,3,5-triazin-2-yl)-1-[2-(morpholin-4-yl)pyrimidin-5-yl]- |
| | 2,3,4,9-tetrahydro-1H-pyrido[3,4-b]indole; |
| 309 | 6-chloro-2-(4,6-dimethyl-1,3,5-triazin-2-yl)-1-[2-(morpholin-4-yl)pyrimidin-5-yl]- |
| | 2,3,4,9-tetrahydro-1H-pyrido[3,4-b]indol-8-amine; |
| 317 | N-butyl-4-{6-chloro-1-[2-(morpholin-4-yl)pyrimidin-5-yl]-1,3,4,9-tetrahydro-2H- |
| | pyrido[3,4-b]indol-2-yl}-N,6-dimethyl-1,3,5-triazin-2-amine; |
| 318 | 2-(4-butoxy-6-methyl-1,3,5-triazin-2-yl)-6-chloro-1-[2-(morpholin-4-yl)pyrimidin-5-yl]- |
| | 2,3,4,9-tetrahydro-1H-pyrido[3,4-b]indole; |
| 321 | 6-chloro-2-(4,6-dimethyl-1,3,5-triazin-2-yl)-1-[6-(morpholin-4-yl)pyridin-3-yl]-2,3,4,9- |
| | tetrahydro-1H-pyrido[3,4-b]indol-8-amine; |
| 346 | 6-chloro-2-(4,6-dimethyl-1,3,5-triazin-2-yl)-1-[2-(methylsulfanyl)pyrimidin-5-yl]- |
| | 2,3,4,9-tetrahydro-1H-pyrido[3,4-b]indole; |
| 347 | (1S)-6-chloro-1-(2,3-difluoro-4-methylphenyl)-2-(4,6-dimethyl-1,3,5-triazin-2-yl)- |
| | 2,3,4,9-tetrahydro-1H-pyrido[3,4-b]indole; |
| 348 | 6-chloro-1-(2,3-difluoro-4-methylphenyl)-2-(4,6-dimethyl-1,3,5-triazin-2-yl)-2,3,4,9- |
| | tetrahydro-1H-pyrido[3,4-b]indol-8-ol; |
| 370 | 6-chloro-2-(4,6-dimethyl-1,3,5-triazin-2-yl)-1-[2-(morpholin-4-yl)pyrimidin-5-yl]- |
| | 2,3,4,9-tetrahydro-1H-pyrido[3,4-b]indol-8-ol; |
| 386 | (1S)-2-[4,6-bis(trifluoromethyl)-1,3,5-triazin-2-yl]-6-chloro-1-(4-methylphenyl)-2,3,4,9- |
| | tetrahydro-1H-pyrido[3,4-b]indole; |
| 387 | (1S)-2-[4,6-bis(trifluoromethyl)-1,3,5-triazin-2-yl]-6-chloro-1-(2,3-difluoro-4- |
| | methylphenyl)-2,3,4,9-tetrahydro-1H-pyrido[3,4-b]indole; |
| 388 | 2-[4,6-bis(trifluoromethyl)-1,3,5-triazin-2-yl]-6-chloro-1-[2-(morpholin-4-yl)pyrimidin- |
| | 5-yl]-2,3,4,9-tetrahydro-1H-pyrido[3,4-b]indole; |
| 389 | (1S)-6-chloro-1-(2,3-difluoro-4-methylphenyl)-2-[4-(methylsulfanyl)-6-(trifluoromethyl)- |
| | 1,3,5-triazin-2-yl]-2,3,4,9-tetrahydro-1H-pyrido[3,4-b]indole; |
| 390 | 4-[(1S)-6-chloro-1-(2,3-difluoro-4-methylphenyl)-1,3,4,9-tetrahydro-2H-pyrido[3,4- |
| | b]indol-2-yl]-6-(trifluoromethyl)-1,3,5-triazine-2-carbonitrile; |
| 402 | (1S)-6-chloro-2-(4,6-dimethyl-1,3,5-triazin-2-yl)-1-(4-methylphenyl)-2,3,4,9-tetrahydro- |
| | 1H-pyrido[3,4-b]indole; |
| 404 | 2-[4,6-bis(trifluoromethyl)-1,3,5-triazin-2-yl]-6-chloro-1-[6-(morpholin-4-yl)pyridin-3- |
| | yl]-2,3,4,9-tetrahydro-1H-pyrido[3,4-b]indole; |
| 405 | (1S)-2-[4,6-bis(trifluoromethyl)-1,3,5-triazin-2-yl]-6-chloro-1-[6-(morpholin-4- |
| | yl)pyridin-3-yl]-2,3,4,9-tetrahydro-1H-pyrido[3,4-b]indole; |
| 406 | (1S)-2-[4,6-bis(trifluoromethyl)-1,3,5-triazin-2-yl]-6-chloro-1-[2-(morpholin-4- |
| | yl)pyrimidin-5-yl]-2,3,4,9-tetrahydro-1H-pyrido[3,4-b]indole; |
| 411 | 4-[(1S)-6-chloro-1-(4-methylphenyl)-1,3,4,9-tetrahydro-2H-pyrido[3,4-b]indol-2-yl]-6- |
| | methyl-1,3,5-triazine-2-carbonitrile; |
| 418 | (1S)-6-chloro-1-(4-methylphenyl)-2-[4-(trifluoromethyl)-1,3,5-triazin-2-yl]-2,3,4,9- |
| | tetrahydro-1H-pyrido[3,4-b]indole; |
| 420 | (1S)-2-[4,6-bis(trifluoromethyl)-1,3,5-triazin-2-yl]-6-chloro-1-[2-(4-methylpiperazin-1- |
| | yl)pyrimidin-5-yl]-2,3,4,9-tetrahydro-1H-pyrido[3,4-b]indole; |
| 428 | 6-chloro-1-[6-(morpholin-4-yl)pyridin-3-yl]-2-[4-(morpholin-4-yl)-6-(trifluoromethyl)- |
| | 1,3,5-triazin-2-yl]-2,3,4,9-tetrahydro-1H-pyrido[3,4-b]indole; |
| 435 | 4-[(1S)-6-chloro-1-(2,3-difluoro-4-methylphenyl)-1,3,4,9-tetrahydro-2H-pyrido[3,4- |
| | b]indol-2-yl]-N,N-dimethyl-6-(trifluoromethyl)-1,3,5-triazin-2-amine; |
| 436 | (1S)-6-chloro-1-(2,3-difluoro-4-methylphenyl)-2-[4-(morpholin-4-yl)-6-(trifluoromethyl)- |
| | 1,3,5-triazin-2-yl]-2,3,4,9-tetrahydro-1H-pyrido[3,4-b]indole; |
| 437 | 6-chloro-1-(4-methylphenyl)-2-[4-methyl-6-(trifluoromethyl)-1,3,5-triazin-2-yl]-2,3,4,9- |
| | tetrahydro-1H-pyrido[3,4-b]indole; |
| 438 | 6-chloro-1-(2,3-difluoro-4-methylphenyl)-2-[4-methyl-6-(trifluoromethyl)-1,3,5-triazin- |
| | 2-yl]-2,3,4,9-tetrahydro-1H-pyrido[3,4-b]indole; |
| 439 | 6-chloro-2-[4-methyl-6-(trifluoromethyl)-1,3,5-triazin-2-yl]-1-[6-(morpholin-4- |
| | yl)pyridin-3-yl]-2,3,4,9-tetrahydro-1H-pyrido[3,4-b]indole; |
| 440 | 6-chloro-2-[4-methyl-6-(trifluoromethyl)-1,3,5-triazin-2-yl]-1-[2-(morpholin-4- |
| | yl)pyrimidin-5-yl]-2,3,4,9-tetrahydro-1H-pyrido[3,4-b]indole; |
| 441 | 4-[(1S)-6-chloro-1-(4-methylphenyl)-1,3,4,9-tetrahydro-2H-pyrido[3,4-b]indol-2-yl]- |
| | N,N-dimethyl-6-(trifluoromethyl)-1,3,5-triazin-2-amine; |
| 442 | (1S)-6-chloro-1-(4-methylphenyl)-2-[4-(morpholin-4-yl)-6-(trifluoromethyl)-1,3,5- |
| | triazin-2-yl]-2,3,4,9-tetrahydro-1H-pyrido[3,4-b]indole; |
| 443 | 4-{(1S)-6-chloro-1-[6-(morpholin-4-yl)pyridin-3-yl]-1,3,4,9-tetrahydro-2H-pyrido[3,4- |
| | b]indol-2-yl}-N,N-dimethyl-6-(trifluoromethyl)-1,3,5-triazin-2-amine; |
| 444 | 4-{(1S)-6-chloro-1-[2-(morpholin-4-yl)pyrimidin-5-yl]-1,3,4,9-tetrahydro-2H-pyrido[3,4- |
| | b]indol-2-yl}-N,N-dimethyl-6-(trifluoromethyl)-1,3,5-triazin-2-amine; |
| 445 | (1S)-6-chloro-1-[2-(morpholin-4-yl)pyrimidin-5-yl]-2-[4-(morpholin-4-yl)-6- |
| | (trifluoromethyl)-1,3,5-triazin-2-yl]-2,3,4,9-tetrahydro-1H-pyrido[3,4-b]indole; |
| 446 | (1S)-6-chloro-1-[6-(morpholin-4-yl)pyridin-3-yl]-2-[4-(morpholin-4-yl)-6- |
| | (trifluoromethyl)-1,3,5-triazin-2-yl]-2,3,4,9-tetrahydro-1H-pyrido[3,4-b]indole; |
| 451 | 6-chloro-1-(2,4-difluorophenyl)-2-(4,6-dimethyl-1,3,5-triazin-2-yl)-2,3,4,9-tetrahydro- |
| | 1H-pyrido[3,4-b]indole; |
| 452 | 6-chloro-2-(4,6-dimethyl-1,3,5-triazin-2-yl)-1-(2-fluoro-4-methylphenyl)-2,3,4,9- |
| | tetrahydro-1H-pyrido[3,4-b]indole; |
| 453 | 4-[6-chloro-2-(4,6-dimethyl-1,3,5-triazin-2-yl)-2,3,4,9-tetrahydro-1H-pyrido[3,4-b]indol- |
| | 1-yl]-3-fluorobenzonitrile; |
| 457 | (1S)-6-chloro-2-[4-(3,3-difluoroazetidin-1-yl)-6-methyl-1,3,5-triazin-2-yl]-1-[2- |
| | (morpholin-4-yl)pyrimidin-5-yl]-2,3,4,9-tetrahydro-1H-pyrido[3,4-b]indole; |
| 459 | 6-chloro-2-(4,6-dimethyl-1,3,5-triazin-2-yl)-1-[2-fluoro-6-(morpholin-4-yl)pyridin-3-yl]- |
| | 2,3,4,9-tetrahydro-1H-pyrido[3,4-b]indole; |
| 460 | (1S)-6-chloro-1-[2-(morpholin-4-yl)pyrimidin-5-yl]-2-[4-(trifluoromethyl)-1,3,5-triazin- |
| | 2-yl]-2,3,4,9-tetrahydro-1H-pyrido[3,4-b]indole; |
| 461 | (1S)-6-chloro-1-(2,3-difluoro-4-methylphenyl)-2-[4-(trifluoromethyl)-1,3,5-triazin-2-yl]- |
| | 2,3,4,9-tetrahydro-1H-pyrido[3,4-b]indole; |
| 462 | (1S)-6-chloro-1-[6-(morpholin-4-yl)pyridin-3-yl]-2-[4-(trifluoromethyl)-1,3,5-triazin-2- |
| | yl]-2,3,4,9-tetrahydro-1H-pyrido[3,4-b]indole; |
| 464 | (1S)-6-chloro-1-(2,3-difluoro-4-methylphenyl)-2-[4-methyl-6-(trifluoromethyl)-1,3,5- |
| | triazin-2-yl]-2,3,4,9-tetrahydro-1H-pyrido[3,4-b]indole; |
| 465 | (1S)-6-chloro-2-[4-methyl-6-(trifluoromethyl)-1,3,5-triazin-2-yl]-1-[6-(morpholin-4- |
| | yl)pyridin-3-yl]-2,3,4,9-tetrahydro-1H-pyrido[3,4-b]indole; |
| 466 | (1S)-6-chloro-2-[4-methyl-6-(trifluoromethyl)-1,3,5-triazin-2-yl]-1-[2-(morpholin-4- |
| | yl)pyrimidin-5-yl]-2,3,4,9-tetrahydro-1H-pyrido[3,4-b]indole; |
| 467 | (1S)-6-chloro-1-(4-methylphenyl)-2-[4-methyl-6-(trifluoromethyl)-1,3,5-triazin-2-yl]- |
| | 2,3,4,9-tetrahydro-1H-pyrido[3,4-b]indole; |
| 468 | 6-chloro-1-(4-chloro-2-fluorophenyl)-2-(4,6-dimethyl-1,3,5-triazin-2-yl)-2,3,4,9- |
| | tetrahydro-1H-pyrido[3,4-b]indole; |
| 474 | (1S)-6-chloro-1-[2-(4-methylpiperazin-1-yl)pyrimidin-5-yl]-2-[4-(methylsulfanyl)-6- |
| | (trifluoromethyl)-1,3,5-triazin-2-yl]-2,3,4,9-tetrahydro-1H-pyrido[3,4-b]indole; |
| 476 | (1S)-6-chloro-1-(2,3-difluoro-4-methylphenyl)-2-[4-fluoro-6-(4-methylpiperazin-1-yl)- |
| | 1,3,5-triazin-2-yl]-2,3,4,9-tetrahydro-1H-pyrido[3,4-b]indole; |
| 477 | (1S)-6-chloro-1-(2,3-difluoro-4-methylphenyl)-2-[4-(4-methylpiperazin-1-yl)-6- |
| | (trifluoromethyl)-1,3,5-triazin-2-yl]-2,3,4,9-tetrahydro-1H-pyrido[3,4-b]indole; |
| 478 | (1S)-6-chloro-2-[4-(4-methylpiperazin-1-yl)-6-(trifluoromethyl)-1,3,5-triazin-2-yl]-1-[2- |
| | (morpholin-4-yl)pyrimidin-5-yl]-2,3,4,9-tetrahydro-1H-pyrido[3,4-b]indole; |
| 486 | (1S)-2-[4,6-bis(trifluoromethyl)-1,3,5-triazin-2-yl]-1-[6-(morpholin-4-yl)pyridin-3-yl]-6- |
| | (2H-1,2,3-triazol-2-yl)-2,3,4,9-tetrahydro-1H-pyrido[3,4-b]indole; |
| 488 | (1S)-6-chloro-1-[2-(4-methylpiperazin-1-yl)pyrimidin-5-yl]-2-[4-(trifluoromethyl)-1,3,5- |
| | triazin-2-yl]-2,3,4,9-tetrahydro-1H-pyrido[3,4-b]indole; |
| 489 | (1S)-6-chloro-1-[6-(4-methylpiperazin-1-yl)pyridin-3-yl]-2-[4-(methylsulfanyl)-6- |
| | (trifluoromethyl)-1,3,5-triazin-2-yl]-2,3,4,9-tetrahydro-1H-pyrido[3,4-b]indole; |
| ¹491 | (1S)-6-chloro-1-[6-(4-methylpiperazin-1-yl)pyridin-3-yl]-2-[4-(trifluoromethyl)-1,3,5- |
| | triazin-2-yl]-2,3,4,9-tetrahydro-1H-pyrido[3,4-b]indole; |
| 492 | 2-[4,6-bis(trifluoromethyl)-1,3,5-triazin-2-yl]-1-(2,3-difluoro-4-methylphenyl)-6-fluoro- |
| | 2,3,4,9-tetrahydro-1H-pyrido[3,4-b]indole; |
| 495 | 1-(2,3-difluoro-4-methylphenyl)-6-fluoro-2-[4-(methylsulfanyl)-6-(trifluoromethyl)- |
| | 1,3,5-triazin-2-yl]-2,3,4,9-tetrahydro-1H-pyrido[3,4-b]indole; |
| 497 | 2-[4,6-bis(trifluoromethyl)-1,3,5-triazin-2-yl]-6-chloro-1-(2,3-difluoro-4-methylphenyl)- |
| | 2,3,4,9-tetrahydro-1H-pyrido[3,4-b]indole; |
| 498 | (1S)-1-[6-(morpholin-4-yl)pyridin-3-yl]-6-(2H-1,2,3-triazol-2-yl)-2-[4-(trifluoromethyl)- |
| | 1,3,5-triazin-2-yl]-2,3,4,9-tetrahydro-1H-pyrido[3,4-b]indole; |
| 501 | (1S)-2-[4,6-bis(trifluoromethyl)-1,3,5-triazin-2-yl]-1-[2-(4-methylpiperazin-1- |
| | yl)pyrimidin-5-yl]-6-(2H-1,2,3-triazol-2-yl)-2,3,4,9-tetrahydro-1H-pyrido[3,4-b]indole; |
| 505 | (1S)-6-chloro-1-(2,3-difluoro-4-methylphenyl)-2-[4-(trichloromethyl)-6- |
| | (trifluoromethyl)-1,3,5-triazin-2-yl]-2,3,4,9-tetrahydro-1H-pyrido[3,4-b]indole; |
| 506 | (1S)-1-[2-(4-methylpiperazin-1-yl)pyrimidin-5-yl]-6-(2H-1,2,3-triazol-2-yl)-2-[4- |
| | (trifluoromethyl)-1,3,5-triazin-2-yl]-2,3,4,9-tetrahydro-1H-pyrido[3,4-b]indole; |
| 507 | (1S)-6-chloro-2-[4-(difluoromethyl)-6-(trifluoromethyl)-1,3,5-triazin-2-yl]-1-[2- |
| | (morpholin-4-yl)pyrimidin-5-yl]-2,3,4,9-tetrahydro-1H-pyrido[3,4-b]indole; |
| 508 | (1S)-6-chloro-2-[4-(difluoromethyl)-6-(trifluoromethyl)-1,3,5-triazin-2-yl]-1-[2-(4- |
| | methylpiperazin-1-yl)pyrimidin-5-yl]-2,3,4,9-tetrahydro-1H-pyrido[3,4-b]indole; |
| 509 | (1S)-6-chloro-1-[2-(4-methylpiperazin-1-yl)pyrimidin-5-yl]-2-[4-(morpholin-4-yl)-6- |
| | (trifluoromethyl)-1,3,5-triazin-2-yl]-2,3,4,9-tetrahydro-1H-pyrido[3,4-b]indole; |
| 510 | (1S)-6-chloro-1-(2,3-difluoro-4-methylphenyl)-2-[4-(difluoromethyl)-6-(trifluoromethyl)- |
| | 1,3,5-triazin-2-yl]-2,3,4,9-tetrahydro-1H-pyrido[3,4-b]indole; |
| 515 | (1S)-1-[2-(morpholin-4-yl)pyrimidin-5-yl]-6-(2H-1,2,3-triazol-2-yl)-2-[4- |
| | (trifluoromethyl)-1,3,5-triazin-2-yl]-2,3,4,9-tetrahydro-1H-pyrido[3,4-b]indole; |
| 516 | (1S)-2-[4,6-bis(trifluoromethyl)-1,3,5-triazin-2-yl]-1-[2-(morpholin-4-yl)pyrimidin-5-yl]- |
| | 6-(2H-1,2,3-triazol-2-yl)-2,3,4,9-tetrahydro-1H-pyrido[3,4-b]indole; |
| 521 | {4-[(1S)-6-chloro-1-(2,3-difluoro-4-methylphenyl)-1,3,4,9-tetrahydro-2H-pyrido[3,4- |
| | b]indol-2-yl]-6-(trifluoromethyl)-1,3,5-triazin-2-yl}methanol; |
| 522 | (1S)-2-[4,6-bis(trifluoromethyl)-1,3,5-triazin-2-yl]-6-chloro-1-(2-methoxypyrimidin-5- |
| | yl)-2,3,4,9-tetrahydro-1H-pyrido[3,4-b]indole; |
| 523 | (1S)-2-[4,6-bis(trifluoromethyl)-1,3,5-triazin-2-yl]-6-bromo-1-(2,3-difluoro-4- |
| | methylphenyl)-2,3,4,9-tetrahydro-1H-pyrido[3,4-b]indole; |
| 524 | (1S)-2-[4,6-bis(trifluoromethyl)-1,3,5-triazin-2-yl]-1-(2,3-difluoro-4-methylphenyl)-6- |
| | (2H-1,2,3-triazol-2-yl)-2,3,4,9-tetrahydro-1H-pyrido[3,4-b]indole; |
| 525 | (1S)-2-[4,6-bis(trifluoromethyl)-1,3,5-triazin-2-yl]-1-(2,3-difluoro-4-methylphenyl)-6-(4- |
| | methyl-2H-1,2,3-triazol-2-yl)-2,3,4,9-tetrahydro-1H-pyrido[3,4-b]indole; |
| 526 | (1S)-6-chloro-1-(2,3-difluoro-4-methylphenyl)-2-[4-(fluoromethyl)-6-(trifluoromethyl)- |
| | 1,3,5-triazin-2-yl]-2,3,4,9-tetrahydro-1H-pyrido[3,4-b]indole; |
| 527 | (1S)-2-[4,6-bis(trifluoromethyl)-1,3,5-triazin-2-yl]-1-(2,3-difluoro-4-methylphenyl)-6- |
| | nitro-2,3,4,9-tetrahydro-1H-pyrido[3,4-b]indole; |
| 529 | (1S)-2-[4,6-bis(trifluoromethyl)-1,3,5-triazin-2-yl]-1-(2,3-difluoro-4-methylphenyl)-6-(4- |
| | methyl-1H-pyrazol-1-yl)-2,3,4,9-tetrahydro-1H-pyrido[3,4-b]indole; |
| 531 | N²-(5-{(1S)-2-[4,6-bis(trifluoromethyl)-1,3,5-triazin-2-yl]-6-chloro-2,3,4,9-tetrahydro- |
| | 1H-pyrido[3,4-b]indol-1-yl}pyrimidin-2-yl)-N¹,N¹-dimethylethane-1,2-diamine; |
| 532 | methyl 5-{(1S)-2-[4,6-bis(trifluoromethyl)-1,3,5-triazin-2-yl]-6-chloro-2,3,4,9-tetrahydro- |
| | 1H-pyrido[3,4-b]indol-1-yl} pyrimidine-2-carboxylate; |
| 533 | 1-(5-{(1S)-2-[4,6-bis(trifluoromethyl)-1,3,5-triazin-2-yl]-6-chloro-2,3,4,9-tetrahydro-1H- |
| | pyrido[3,4-b]indol-1-yl}pyrimidin-2-yl)ethan-1-one; |
| 534 | (1S)-2-[4,6-bis(trifluoromethyl)-1,3,5-triazin-2-yl]-6-chloro-1-[2-(2- |
| | methoxyethoxy)pyrimidin-5-yl]-2,3,4,9-tetrahydro-1H-pyrido[3,4-b]indole; |
| 535 | 2-[(5-{(1S)-2-[4,6-bis(trifluoromethyl)-1,3,5-triazin-2-yl]-6-chloro-2,3,4,9-tetrahydro- |
| | 1H-pyrido[3,4-b]indol-1-yl}pyrimidin-2-yl)amino]ethan-1-ol; |
| 536 | 2-[4,6-bis(trifluoromethyl)-1,3,5-triazin-2-yl]-6-chloro-1-[2-(2- |
| | methoxyethoxy)pyrimidin-5-yl]-2,3,4,9-tetrahydro-1H-pyrido[3,4-b]indole; |
| 537 | N-(5-{(1S)-2-[4,6-bis(trifluoromethyl)-1,3,5-triazin-2-yl]-6-chloro-2,3,4,9-tetrahydro-1H- |
| | pyrido[3,4-b]indol-1-yl}pyrimidin-2-yl)-β-alanine; and, |
| 538 | 4-[(5-{(1S)-2-[4,6-bis(trifluoromethyl)-1,3,5-triazin-2-yl]-6-chloro-2,3,4,9-tetrahydro- |
| | 1H-pyrido[3,4-b]indol-1-yl}pyrimidin-2-yl)amino]butanoic acid; |

wherein the form of the compound is selected from the group consisting of a salt, hydrate solvate, racemate, enantiomer, diastereomer, stereoisomer, and tautomer form thereof.

Another aspect of the compound of Formula (I) or a form thereof includes a compound (Cpd) salt having dihydroorotate dehydrogenase inhibition activity ≤100 nM selected from the group consisting of:

| Cpd | Name |
|---|---|
| 491 | (1S)-6-chloro-1-[6-(4-methylpiperazin-1-yl)pyridin-3-yl]-2-[4-(trifluoromethyl)-1,3,5- |
| | triazin-2-yl]-2,3,4,9-tetrahydro-1H-py.rido[3,4-b]indole formate (1:1); |

wherein the form of the compound is selected from the group consisting of a hydrate, solvate, racemate, enantiomer, diastereomer, stereoisomer, and tautomer form thereof.

One aspect of the compound of Formula (I) or a form thereof includes a compound (Cpd) having dihydroorotate dehydrogenase inhibition activity ≤1.0 nM selected from the group consisting of (wherein ¹Cpd #indicates the compound was isolated as a salt form):

| Cpd | Name |
|---|---|
| 283 | 1-(4-bromo-2,3-difluorophenyl)-6-chloro-2-(4,6-dimethyl-1,3,5-triazin-2-yl)-2,3,4,9- |
| | tetrahydro-1H-pyrido[3,4-b]indole; |
| 386 | (1S)-2-[4,6-bis(trifluoromethyl)-1,3,5-triazin-2-yl]-6-chloro-1-(4-methylphenyl)- |
| | 2,3,4,9-tetrahydro-1H-pyrido[3,4-b]indole; |
| 387 | (1S)-2-[4,6-bis(trifluoromethyl)-1,3,5-triazin-2-yl]-6-chloro-1-(2,3-difluoro-4- |
| | methylphenyl)-2,3,4,9-tetrahydro-1H-pyrido[3,4-b]indole; |
| 388 | 2-[4,6-bis(trifluoromethyl)-1,3,5-triazin-2-yl]-6-chloro-1-[2-(morpholin-4- |
| | yl)pyrimidin-5-yl]-2,3,4,9-tetrahydro-1H-pyrido[3,4-b]indole; |
| 389 | (1S)-6-chloro-1-(2,3-difluoro-4-methylphenyl)-2-[4-(methylsulfanyl)-6- |
| | (trifluoromethyl)-1,3,5-triazin-2-yl]-2,3,4,9-tetrahydro-1H-pyrido[3,4-b]indole; |
| 404 | 2-[4,6-bis(trifluoromethyl)-1,3,5-triazin-2-yl]-6-chloro-1-[6-(morpholin-4-yl)pyridin- |
| | 3-yl]-2,3,4,9-tetrahydro-1H-pyrido[3,4-b]indole; |
| 405 | (1S)-2-[4,6-bis(trifluoromethyl)-1,3,5-triazin-2-yl]-6-chloro-1-[6-(morpholin-4- |
| | yl)pyridin-3-yl]-2,3,4,9-tetrahydro-1H-pyrido[3,4-b]indole; |
| 406 | (1S)-2-[4,6-bis(trifluoromethyl)-1,3,5-triazin-2-yl]-6-chloro-1-[2-(morpholin-4- |
| | yl)pyrimidin-5-yl]-2,3,4,9-tetrahydro-1H-pyrido[3,4-b]indole; |
| 420 | (1S)-2-[4,6-bis(trifluoromethyl)-1,3,5-triazin-2-yl]-6-chloro-1-[2-(4-methylpiperazin- |
| | 1-yl)pyrimidin-5-yl]-2,3,4,9-tetrahydro-1H-pyrido[3,4-b]indole; |
| 436 | (1S)-6-chloro-1-(2,3-difluoro-4-methylphenyl)-2-[4-(morpholin-4-yl)-6- |
| | (trifluoromethyl)-1,3,5-triazin-2-yl]-2,3,4,9-tetrahydro-1H-pyrido[3,4-b]indole; |
| 438 | 6-chloro-1-(2,3-difluoro-4-methylphenyl)-2-[4-methyl-6-(trifluoromethyl)-1,3,5- |
| | triazin-2-yl]-2,3,4,9-tetrahydro-1H-pyrido[3,4-b]indole; |
| 461 | (1S)-6-chloro-1-(2,3-difluoro-4-methylphenyl)-2-[4-(trifluoromethyl)-1,3,5-triazin-2- |
| | yl]-2,3,4,9-tetrahydro-1H-pyrido[3,4-b]indole; |
| 464 | (1S)-6-chloro-1-(2,3-difluoro-4-methylphenyl)-2-[4-methyl-6-(trifluoromethyl)-1,3,5- |
| | triazin-2-yl]-2,3,4,9-tetrahydro-1H-pyrido[3,4-b]indole; |
| 465 | (1S)-6-chloro-2-[4-methyl-6-(trifluoromethyl)-1,3,5-triazin-2-yl]-1-[6-(morpholin-4- |
| | yl)pyridin-3-yl]-2,3,4,9-tetrahydro-1H-pyrido[3,4-b]indole; |
| 466 | (1S)-6-chloro-2-[4-methyl-6-(trifluoromethyl)-1,3,5-triazin-2-yl]-1-[2-(morpholin-4- |
| | yl)pyrimidin-5-yl]-2,3,4,9-tetrahydro-1H-pyrido[3,4-b]indole; |
| 467 | (1S)-6-chloro-1-(4-methylphenyl)-2-[4-methyl-6-(trifluoromethyl)-1,3,5-triazin-2-yl]- |
| | 2,3,4,9-tetrahydro-1H-pyrido[3,4-b]indole; |
| 486 | (1S)-2-[4,6-bis(trifluoromethyl)-1,3,5-triazin-2-yl]-1-[6-(morpholin-4-yl)pyridin-3- |
| | yl]-6-(2H-1,2,3-triazol-2-yl)-2,3,4,9-tetrahydro-1H-pyrido[3,4-b]indole; |
| 497 | 2-[4,6-bis(trifluoromethyl)-1,3,5-triazin-2-yl]-6-chloro-1-(2,3-difluoro-4- |
| | methylphenyl)-2,3,4,9-tetrahydro-1H-pyrido[3,4-b]indole; |
| 498 | (1S)-1-[6-(morpholin-4-yl)pyridin-3-yl]-6-(2H-1,2,3-triazol-2-yl)-2-[4- |
| | (trifluoromethyl)-1,3,5-triazin-2-yl]-2,3,4,9-tetrahydro-1H-pyrido[3,4-b]indole; |
| 507 | (1S)-6-chloro-2-[4-(difluoromethyl)-6-(trifluoromethyl)-1,3,5-triazin-2-yl]-1-[2- |
| | (morpholin-4-yl)pyrimidin-5-yl]-2,3,4,9-tetrahydro-1H-pyrido[3,4-b]indole; |
| 508 | (1S)-6-chloro-2-[4-(difluoromethyl)-6-(trifluoromethyl)-1,3,5-triazin-2-yl]-1-[2-(4- |
| | methylpiperazin-1-yl)pyrimidin-5-yl]-2,3,4,9-tetrahydro-1H-pyrido[3,4-b]indole; |
| 510 | (1S)-6-chloro-1-(2,3-difluoro-4-methylphenyl)-2-[4-(difluoromethyl)-6- |
| | (trifluoromethyl)-1,3,5-triazin-2-yl]-2,3,4,9-tetrahydro-1H-pyrido[3,4-b]indole; |
| 522 | (1S)-2-[4,6-bis(trifluoromethyl)-1,3,5-triazin-2-yl]-6-chloro-1-(2-methoxypyrimidin- |
| | 5-yl)-2,3,4,9-tetrahydro-1H-pyrido[3,4-b]indole; |
| 523 | (1S)-2-[4,6-bis(trifluoromethyl)-1,3,5-triazin-2-yl]-6-bromo-1-(2,3-difluoro-4- |
| | methylphenyl)-2,3,4,9-tetrahydro-1H-pyrido[3,4-b]indole; |
| 524 | (1S)-2-[4,6-bis(trifluoromethyl)-1,3,5-triazin-2-yl]-1-(2,3-difluoro-4-methylphenyl)-6- |
| | (2H-1,2,3-triazol-2-yl)-2,3,4,9-tetrahydro-1H-pyrido[3,4-b]indole; |
| 526 | (1S)-6-chloro-1-(2,3-difluoro-4-methylphenyl)-2-[4-(fluoromethyl)-6- |
| | (trifluoromethyl)-1,3,5-triazin-2-yl]-2,3,4,9-tetrahydro-1H-pyrido[3,4-b]indole; |
| 527 | (1S)-2-[4,6-bis(trifluoromethyl)-1,3,5-triazin-2-yl]-1-(2,3-difluoro-4-methylphenyl)-6- |
| | nitro-2,3,4,9-tetrahydro-1H-pyrido[3,4-b]indole; |
| 534 | (1S)-2-[4,6-bis(trifluoromethyl)-1,3,5-triazin-2-yl]-6-chloro-1-[2-(2- |
| | methoxyethoxy)pyrimidin-5-yl]-2,3,4,9-tetrahydro-1H-pyrido[3,4-b]indole; |
| 535 | 2-[(5-{(1S)-2-[4,6-bis(trifluoromethyl)-1,3,5-triazin-2-yl]-6-chloro-2,3,4,9-tetrahydro- |
| | 1H-pyrido[3,4-b]indol-1-yl}pyrimidin-2-yl)amino]ethan-1-ol; and, |
| 538 | 4-[(5-{(1S)-2-[4,6-bis(trifluoromethyl)-1,3,5-triazin-2-yl]-6-chloro-2,3,4,9-tetrahydro- |
| | 1H-pyrido[3,4-b]indol-1-yl}pyrimidin-2-yl)amino]butanoic acid; |

wherein the form of the compound is selected from the group consisting of a salt, hydrate, solvate, racemate, enantiomer, diastereomer, stereoisomer, and tautomer form thereof.

### Definitions

The chemical terms used above and throughout the description herein, unless specifically defined otherwise, shall be understood by one of ordinary skill in the art to have the following indicated meanings.

As used herein, the term “C₁₋₄alkyl”, “C₁₋₆alkyl”, or “C₁₋₈alkyl” generally refers to saturated hydrocarbon radicals having from up to eight carbon atoms in a straight or branched chain configuration, including, but not limited to, methyl, ethyl, n-propyl (also referred to as propyl or propanyl), isopropyl, n-butyl (also referred to as butyl or butanyl), isobutyl, sec-butyl, tert-butyl, n-pentyl (also referred to as pentyl or pentanyl), n-hexyl (also referred to as hexyl or hexanyl), n-heptyl (also referred to as heptyl or heptanyl), n-octyl (also referred to as octyl or octanyl) and the like. A C₁₋₄alkyl, C₁₋₆alkyl, or C₁₋₈alkyl radical is optionally substituted with substituent species as described herein, where allowed by available valences.

As used herein, the term “C₂₋₄alkenyl”, “C₂₋₆alkenyl”, or “C₂₋₈alkenyl” generally refers to partially unsaturated hydrocarbon radicals having from two up to eight carbon atoms in a straight or branched chain configuration and one or more carbon-carbon double bonds therein, including, but not limited to, ethenyl (also referred to as vinyl), allyl, propenyl and the like. A C₂₋₄alkenyl, C₂₋₆alkenyl, or C₂₋₈alkenyl radical is optionally substituted with substituent species as described herein, where allowed by available valences.

As used herein, the term “C₁₋₄alkoxy”, “C₁₋₆alkoxy”, or “C₁₋₈alkoxy” generally refers to saturated hydrocarbon radicals having from one to eight carbon atoms in a straight or branched chain configuration of the formula: —O—C₁₋₄alkyl, —O—C₁₋₆alkyl, —O—C₁₋₈alkyl, including, but not limited to, methoxy, ethoxy, n-propoxy, isopropoxy, n-butoxy, isobutoxy, sec-butoxy, tert-butoxy and the like. A C₁₋₄alkoxy, C₁₋₆alkoxy, C₁₋₈alkoxy radical is optionally substituted with substituent species as described herein where allowed by available valences.

As used herein, the term “C₃₋₆cycloalkyl” generally refers to a saturated or partially unsaturated monocyclic, bicyclic or polycyclic hydrocarbon ring system radical, including, but not limited to, cyclopropyl, cyclobutyl, cyclopentyl, cyclohexyl, cyclohexenyl, and the like. In certain aspects, C₃₋₆cycloalkyl includes, but is not limited to C₃cycloalkyl, C₄cycloalkyl, C₅₋₆cycloalkyl and the like. A C₃₋₆cycloalkyl ring system radical is optionally substituted with substituent species as described herein where allowed by available valences.

As used herein, the term “aryl” generally refers to a monocyclic, bicyclic or polycyclic aromatic carbon atom ring system radical, including, but not limited to, phenyl, naphthyl, anthracenyl, fluorenyl, azulenyl, phenanthrenyl and the like. In certain aspects, the aryl monocyclic, or bicyclic ring system radical is phenyl, or naphthyl, respectively. An aryl ring system radical is optionally substituted with substituent species as described herein where allowed by available valences.

As used herein, the term “heteroaryl” generally refers to a monocyclic aromatic carbon atom ring system radical in which one or more carbon atom ring members have been replaced, where allowed by structural stability, with one or more heteroatoms, such as an N, O, or S atom. In certain aspects, a heteroaryl ring system radical may have one, two, or three carbon atom ring members replaced with a heteroatom ring member independently selected from N, O, or S. In certain aspects, a heteroaryl ring system radical may be optionally substituted with one, two, or three substituents where allowed by available valences. In certain aspects, a heteroaryl ring system radical may include a monocyclic, bicyclic or polycyclic carbon atom ring system radical, such as a 5-6 membered monocyclic, 7-8 membered monocyclic or 9-10 membered bicyclic ring system.

In certain aspects, a heteroaryl radical may include a ring system radical such as, but not limited to, furanyl, thienyl, pyrrolyl, pyrazolyl, imidazolyl, isoxazolyl, isothiazolyl, oxazolyl, 1,3-thiazolyl, triazolyl, oxadiazolyl, thiadiazolyl, tetrazolyl, pyridinyl, pyrimidinyl, pyrazinyl, pyridazinyl, triazinyl, indolyl, indazolyl, indolizinyl, isoindolyl. A heteroaryl radical is optionally substituted on a carbon or nitrogen atom ring member with substituent species as described herein where allowed by available valences.

In certain aspects, the nomenclature for a heteroaryl radical may differ, such as in non-limiting examples where furanyl may also be referred to as furyl, thienyl may also be referred to as thiophenyl, and pyridinyl may also be referred to as pyridyl.

In certain aspects, the term for a heteroaryl radical may also include other regioisomers, such as in non-limiting examples where the term pyrrolyl may also include 1H-pyrrolyl, 2H-pyrrolyl, 3H-pyrrolyl and the like; the term pyrazolyl may also include 1H-pyrazolyl and the like; the term imidazolyl may also include 1H-imidazolyl and the like; the term triazolyl may also include 1H-1,2,3-triazolyl, 2H-1,2,3-triazolyl and the like; the term oxadiazolyl may also include 1,2,4-oxadiazolyl, 1,3,4-oxadiazolyl and the like; the term tetrazolyl may also include 1H-tetrazolyl, 2H-tetrazolyl and the like; the term indolyl may also include 1H-indolyl and the like; the term indazolyl may also include 1H-indazolyl, 2H-indazolyl and the like; the term benzoimidazolyl may also include 1H-benzoimidazolyl; and the term purinyl may also include 9H-purinyl and the like.

As used herein, the term “heterocyclyl” generally refers to a saturated or partially unsaturated monocyclic, bicyclic or polycyclic carbon atom ring system radical in which one or more carbon atom ring members have been replaced, where allowed by structural stability, with a heteroatom, such as an O, S or N atom. In certain aspects, a heterocyclyl ring system radical may have one, two, or three carbon atom ring members replaced with a heteroatom ring member independently selected from N, O, or S. In certain aspects, a heterocyclyl ring system radical may be optionally substituted with one, two, or three substituents where allowed by available valences. In certain aspects, a heterocyclyl radical may include a monocyclic, bicyclic or polycyclic carbon atom ring system radical, such as a 3-7 membered monocyclic, 7-8 membered bicyclic, 6-10 membered bicyclic or 13-16 membered polycyclic ring system.

In certain aspects, a heterocyclyl radical may include ring system radical such as, but not limited to, oxiranyl, oxetanyl, azetidinyl, tetrahydrofuranyl, pyrrolinyl, pyrrolidinyl, pyrazolinyl, pyrazolidinyl, imidazolinyl, imidazolidinyl, isoxazolinyl, isoxazolidinyl, isothiazolinyl, isothiazolidinyl, oxazolinyl, oxazolidinyl, thiazolinyl, thiazolidinyl, triazolinyl, triazolidinyl, oxadiazolinyl, oxadiazolidinyl, thiadiazolinyl, thiadiazolidinyl, tetrazolinyl, tetrazolidinyl, pyranyl, dihydro-2H-pyranyl, thiopyranyl, 1,3-dioxanyl, 1,2,5,6-tetrahydropyridinyl, 1,2,3,6-tetrahydropyridinyl, piperidinyl, piperazinyl, morpholinyl, thiomorpholinyl, 1,4-diazepanyl, 1,3-benzodioxolyl, 1,4-benzodioxanyl, 2,3-dihydro-1,4-benzodioxinyl, hexahydropyrrolo[3,4-b]pyrrol-(1H)-yl, (3aS,6aS)-hexahydropyrrolo[3,4-b]pyrrol-(1H)-yl, (3aR,6aR)-hexahydropyrrolo[3,4-b]pyrrol-(1H)-yl, hexahydropyrrolo[3,4-b]pyrrol-(2H)-yl, (3aS,6aS)-hexahydropyrrolo[3,4-b]pyrrol-(2H)-yl, (3aR,6aR)-hexahydropyrrolo[3,4-b]pyrrol-(2H)-yl, hexahydropyrrolo[3,4-c]pyrrol-(1H)-yl, (3aR,6aS)-hexahydropyrrolo[3,4-c]pyrrol-(1H)-yl, (3aR,6aR)-hexahydropyrrolo[3,4-c]pyrrol-(1H)-yl, octahydro-5H-pyrrolo[3,2-c]pyridinyl, octahydro-6H-pyrrolo[3,4-b]pyridinyl, (4aR,7aR)-octahydro-6H-pyrrolo[3,4-b]pyridinyl, (4aS,7aS)-octahydro-6H-pyrrolo[3,4-b]pyridinyl, hexahydropyrrolo[1,2-a]pyrazin-(1H)-yl, (7R,8aS)-hexahydropyrrolo[1,2-a]pyrazin-(1H)-yl, (8aS)-hexahydropyrrolo[1,2-a]pyrazin-(1H)-yl, (8aR)-hexahydropyrrolo[1,2-a]pyrazin-(1H)-yl, (8aS)-octahydropyrrolo[1,2-a]pyrazin-(1H)-yl, (8aR)-octahydropyrrolo[1,2-a]pyrazin-(1H)-yl, hexahydropyrrolo[1,2-a]pyrazin-(2H)-one, octahydro-2H-pyrido[1,2-a]pyrazinyl, 3-azabicyclo[3.1.0]hexyl, (1R,5S)-3-azabicyclo[3.1.0]hexyl, 3-oxa-8-azabicyclo[3.2.1]octanyl, 8-azabicyclo[3.2.1]octyl, (1R,5S)-8-azabicyclo[3.2.1]octyl, 8-azabicyclo[3.2.1]oct-2-enyl, (1R,5S)-8-azabicyclo[3.2.1]oct-2-enyl, 9-azabicyclo[3.3.1]nonyl, (1R,5S)-9-azabicyclo[3.3.1]nonyl, 2,5-diazabicyclo[2.2.1]heptyl, 2-oxa-5-azabicyclo[2.2.1]heptanyl, (1R,4R)-2-oxa-5-azabicyclo[2.2.1]heptanyl, (1S,4S)-2-oxa-5-azabicyclo[2.2.1]heptanyl, (1S,4S)-2,5-diazabicyclo[2.2.1]heptyl, 2,5-diazabicyclo[2.2.2]octyl, 3,8-diazabicyclo[3.2.1]octyl, (1R,5S)-3,8-diazabicyclo[3.2.1]octyl, 1,4-diazabicyclo[3.2.2]nonyl, 4,7-diazaspiro[2.5]octanyl. azaspiro[3.3]heptyl, 2-oxa-6-azaspiro[3.3]heptanyl, 2,6-diazaspiro[3.3]heptyl, 2,7-diazaspiro[3.5]nonyl, 5,8-diazaspiro[3.5]nonyl, 2,7-diazaspiro[4.4]nonyl, 6,9-diazaspiro[4.5]decyl and the like.

A heterocyclyl radical is optionally substituted on a carbon or nitrogen atom ring member with substituent species as described herein where allowed by available valences.

In certain aspects, the heterocyclyl radical 1H-imidazol-1-yl may be shown as

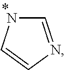

wherein the asterisk (*) designates the point of attachment.

In certain aspects, the heterocyclyl radical 1H-pyrazol-1-yl may be shown as

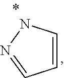

wherein the asterisk (*) designates the point of attachment.

In certain aspects, the heterocyclyl radical 1H-1,2,4-triazol-1-yl may be shown as

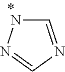

wherein the asterisk (*) designates the point of attachment.

In certain aspects, the heterocyclyl radical morpholin-4-yl may be shown as

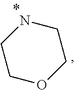

wherein the asterisk (*) designates the point of attachment.

In certain aspects, the heterocyclyl radical piperazin-1-yl may be shown as

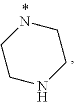

wherein the asterisk (*) designates the point of attachment.

In certain aspects, the heterocyclyl radical 4-methyl-piperazin-1-yl may be shown as

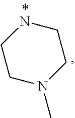

wherein the asterisk (*) designates the point of attachment.

In certain aspects, the heterocyclyl radical azetidin-1-yl may be shown as

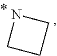

wherein the asterisk (*) designates the point of attachment.

In certain aspects, the heterocyclyl radical 2-oxa-5-azabicyclo[2.2.1]heptan-5-yl may be shown as

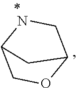

wherein the asterisk (*) designates the point of attachment.

In certain aspects, the heterocyclyl radical 3-oxa-8-azabicyclo[3.2.1]octan-8-yl may be shown as

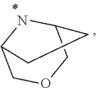

wherein the asterisk (*) designates the point of attachment.

In certain aspects, the heterocyclyl radical (1S,4S)-2-oxa-5-azabicyclo[2.2.1]heptan-5-yl may be shown as

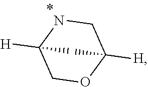

wherein the asterisk (*) designates the point of attachment.

In certain aspects, the heterocyclyl radical (R,4R)-2-oxa-5-azabicyclo[2.2]heptan-5-yl may be shown as

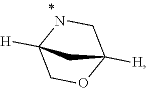

wherein the asterisk (*) designates the point of attachment.

In certain aspects, the heterocyclyl radical 2-oxa-6-azaspiro[3.3]heptan-6-yl may be shown as

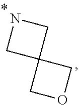

wherein the asterisk (*) designates the point of attachment.

In certain aspects, the heterocyclyl radical 3,3-dimethyl-piperazin-1-yl may be shown as

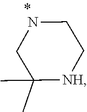

wherein the asterisk (*) designates the point of attachment.

In certain aspects, the heterocyclyl radical (3R,5S)-3,5-dimethylpiperazin-1-yl may be shown as

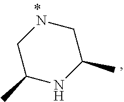

wherein the asterisk (*) designates the point of attachment.

In certain aspects, the heterocyclyl radical 4,7-diazaspiro[2.5]octan-7-yl may be shown as

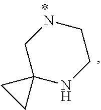

wherein the asterisk (*) designates the point of attachment.

In certain aspects, the heterocyclyl radical (3R)-3-methylpiperazin-1-yl may be shown as

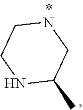

wherein the asterisk (*) designates the point of attachment.

In certain aspects, the heterocyclyl radical (3S)-3-methylpiperazin-1-yl may be shown as

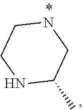

wherein the asterisk (*) designates the point of attachment.

As used herein, the term “C₁₋₄alkoxy-C₁₋₄alkyl” refers to a radical of the formula: —C₁₋₆alkyl-O—C₁₋₆alkyl.

As used herein, the term “C₁₋₄alkoxy-C₁₋₄alkoxy” refers to a radical of the formula: —O—C₁₋₄alkyl-O—C₁₋₄alkyl. In certain aspects, C₁₋₄alkoxy-C₁₋₄alkoxy includes, but is not limited to methoxy-ethoxy and the like.

As used herein, the term “C₁₋₄alkoxycarbonyl” or “(C₁₋₄alkoxy)carbonyl” refers to a radical of the formula: —C(═O)—O—C₁₋₄alkyl. In certain aspects, C₁₋₄alkoxycarbonyl includes, but is not limited to methoxy-carbonyl and the like.

As used herein, the term “C₁₋₄alkoxycarbonylC₁₋₄alkyl” or “(C₁₋₄alkoxy)carbonylC₁₋₄alkyl” refers to a radical of the formula: —C₁₋₄alkyl-C(═O)—O—C₁₋₄alkyl. In certain aspects, C₁₋₄alkoxycarbonylC₁₋₄alkyl includes, but is not limited to methoxy-carbonyl-ethyl and the like.

As used herein, the term “amino” refers to a radical of the formula: —NH₂.

As used herein, the term “amino-sulfonyl” refers to a radical of the formula: —SO₂—NH₂ or —S(═O)(═O)—NH₂.

As used herein, the term “(C₁₋₄alkyl)amino” refers to a radical of the formula: —NH—C₁₋₄alkyl. In certain aspects, (C₁₋₄alkyl)amino includes, but is not limited to N-methyl-amino and the like.

As used herein, the term “(C₁₋₄alkyl)₂amino” refers to a radical of the formula: —N(C₁₋₄alkyl)₂, where each C₁₋₄alkyl can be the same or different. In certain aspects, (C₁₋₄alkyl)₂-amino includes, but is not limited to N,N-dimethyl-amino and the like.

As used herein, the term “(C₁₋₄alkyl)₂amino-(C₁₋₄alkyl)amino” refers to a radical of the formula —NH—C₁₋₄alkyl-NH—(C₁₋₄alkyl)₂. In certain aspects, (C₁₋₄alkyl)₂amino-(C₁₋₄alkyl)amino includes but is not limited to N[N,N-dimethylamino)ethyl]amino and the like.

As used herein, the term “C₁₋₄alkylcarbonyl” refers to a radical of the formula: —C(═O)—C₁₋₄alkyl. In certain aspects, C₁₋₄alkylcarbonyl includes, but is not limited to methyl-carbonyl and the like.

As used herein, the term “C₁₋₄alkyl-sulfonyl” refers to a radical of the formula: —SO₂—C₁₋₄alkyl, —S(═O)(═O)—C₁₋₄alkyl or —S(═O)₂—C₁₋₄alkyl. In certain aspects, C₁₋₄alkyl-sulfonyl includes, but is not limited to methyl-sulfonyl.

As used herein, the term “C₁₋₄alkylthio” refers to a radical of the formula: —S—C₁₋₄alkyl. C₁₋₄alkylthio includes but is not limited to methyl-thio and the like.

As used herein, the term “amino-C₁₋₄alkyl” refers to a radical of the formula: —C₁₋₄alkyl-NH₂.

As used herein, the term “carboxylC₁₋₄alkyl” refers to a radical of the formula: —C₁₋₄alkyl-C(═O)—OH.

As used herein, the term “carboxylC₁₋₄alkylamino” refers to a radical of the formula: —NH—C₁₋₄alkyl-C(═O)—OH.

As used herein, the term “carboxylC₁₋₄alkoxyC₁₋₄alkylamino” refers to a radical of the formula: —NH—C₁₋₄alkyl-O—C₁₋₄alkyl-C(═O)—OH.

As used herein, the term “halo” or “halogen” generally refers to a halogen atom radical, including fluoro, chloro, bromo and iodo.

As used herein, the term “halo-C₁₋₄alkoxy” refers to a radical of the formula: —O—C₁₋₄alkyl-halo, wherein C₁₋₄alkyl is partially or completely substituted with one or more halogen atoms where allowed by available valences.

As used herein, the term “halo-C₁₋₄alkyl” refers to a radical of the formula: —C₁₋₄alkyl-halo, wherein C₁₋₄alkyl is partially or completely substituted with one or more halogen atoms where allowed by available valences. In certain aspects, halo-C₁₋₄alkyl includes, but is not limited to fluoromethyl, difluoromethyl, trifluoromethyl and the like.

As used herein, the term “hydroxy” refers to a radical of the formula: —OH.

As used herein, the term “hydroxy-C₁₋₄alkyl” refers to a radical of the formula: —C₁₋₄alkyl-OH, wherein C₁₋₄alkyl is partially or completely substituted with one or more hydroxy radicals where allowed by available valences. In certain aspects, hydroxy-C₁₋₄alkyl includes, but is not limited to hydroxy-methyl and the like.

As used herein, the term “hydroxy-C₂₋₄alkenyl” refers to a radical of the formula: —C₂₋₄alkenyl-OH, wherein C₂₋₄alkenyl is partially or completely substituted with one or more hydroxy radicals where allowed by available valences.

As used herein, the term “hydroxyC₁₋₄alkylamino” refers to a radical of the formula: —NH—C₁₋₄alkyl-OH, wherein C₁₋₄alkyl is partially or completely substituted with one or more hydroxy radicals where allowed by available valences. In certain aspects, hydroxyC₁₋₄alkylamino includes, but is not limited to N-(hydroxyethyl)amino and the like.

As used herein, the term “hydroxyC₁₋₄alkoxyC₁₋₄alkyl” refers to a radical of the formula —C₁₋₄alkyl-O—C₁₋₄alkyl-OH.

As used herein, the term “substituent” means positional variables on the atoms of a core molecule that are substituted at a designated atom position, replacing one or more hydrogens on the designated atom, provided that the designated atom's normal valency is not exceeded, and that the substitution results in a stable compound. Combinations of substituents and/or variables are permissible only if such combinations result in stable compounds. A person of ordinary skill in the art should note that any carbon as well as heteroatom with valences that appear to be unsatisfied as described or shown herein is assumed to have a sufficient number of hydrogen atom(s) to satisfy the valences described or shown. In certain instances one or more substituents having a double bond (e.g., “oxo” or “═O”) as the point of attachment may be described, shown or listed herein within a substituent group, wherein the structure may only show a single bond as the point of attachment to the core structure of Formula (I). A person of ordinary skill in the art would understand that, while only a single bond is shown, a double bond is intended for those substituents.

As used herein, the term “and the like,” with reference to the definitions of chemical terms provided herein, means that variations in chemical structures that could be expected by one skilled in the art include, without limitation, isomers (including chain, branching or positional structural isomers), hydration of ring systems (including saturation or partial unsaturation of monocyclic, bicyclic or polycyclic ring structures) and all other variations where allowed by available valences which result in a stable compound.

For the purposes of this description, where one or more substituent variables for a compound of Formula (I) or a form thereof encompass functionalities incorporated into a compound of Formula (I), each functionality appearing at any location within the disclosed compound may be independently selected, and as appropriate, independently and/or optionally substituted.

As used herein, the terms “independently selected,” or “each selected” refer to functional variables in a substituent list that may occur more than once on the structure of Formula (I), the pattern of substitution at each occurrence is independent of the pattern at any other occurrence. Further, the use of a generic substituent variable on any formula or structure for a compound described herein is understood to include the replacement of the generic substituent with species substituents that are included within the particular genus, e.g., aryl may be replaced with phenyl or naphthalenyl and the like, and that the resulting compound is to be included within the scope of the compounds described herein.

As used herein, the terms “each instance of” or “in each instance, when present,” when used preceding a phrase such as “ . . . C₃₋₁₄cycloalkyl, C₃₋₁₄cycloalkyl-C₁₋₄alkyl, aryl, aryl-C₁₋₄alkyl, heteroaryl, heteroaryl-C₁₋₄alkyl, heterocyclyl and heterocyclyl-C₁₋₄alkyl,” are intended to refer to the C₃₋₁₄cycloalkyl, aryl, heteroaryl and heterocyclyl ring systems when each are present either alone or as a substituent.

As used herein, the term “optionally substituted” means optional substitution with the specified substituent variables, groups, radicals or moieties.

### Compound Forms

As used herein, the term “form” means a compound of Formula (I) having a form selected from the group consisting of a free acid, free base, salt, hydrate, solvate, racemate, enantiomer, diastereomer, stereoisomer, and tautomer form thereof.

In certain aspects described herein, the form of the compound of Formula (I) is a free acid, free base or salt thereof.

In certain aspects described herein, the form of the compound of Formula (I) is a salt thereof.

In certain aspects described herein, the form of the compound of Formula (I) is an isotopologue thereof.

In certain aspects described herein, the form of the compound of Formula (I) is a stereoisomer, racemate, enantiomer or diastereomer thereof.

In certain aspects described herein, the form of the compound of Formula (I) is a tautomer thereof.

In certain aspects described herein, the form of the compound of Formula (I) is a pharmaceutically acceptable form.

In certain aspects described herein, the compound of Formula (I) or a form thereof is isolated for use.

As used herein, the term “isolated” means the physical state of a compound of Formula (I) or a form thereof after being isolated and/or purified from a synthetic process (e.g., from a reaction mixture) or natural source or combination thereof according to an isolation or purification process or processes described herein or which are well known to the skilled artisan (e.g., chromatography, recrystallization and the like) in sufficient purity to be characterized by standard analytical techniques described herein or well known to the skilled artisan.

As used herein, the term “protected” means that a functional group in a compound of Formula (I) or a form thereof is in a form modified to preclude undesired side reactions at the protected site when the compound is subjected to a reaction. Suitable protecting groups will be recognized by those with ordinary skill in the art as well as by reference to standard textbooks such as, for example, T. W. Greene et al, Protective Groups in organic Synthesis (1991), Wiley, New York. Such functional groups include hydroxy, phenol, amino and carboxylic acid. Suitable protecting groups for hydroxy or phenol include trialkylsilyl or diarylalkylsilyl (e.g., t-butyldimethylsilyl, t-butyldiphenylsilyl or trimethylsilyl), tetrahydropyranyl, benzyl, substituted benzyl, methyl, methoxymethanol, and the like. Suitable protecting groups for amino, amidino and guanidino include t-butoxycarbonyl, benzyloxycarbonyl, and the like. Suitable protecting groups for carboxylic acid include alkyl, aryl or arylalkyl esters. In certain instances, the protecting group may also be a polymer resin, such as a Wang resin or a 2-chlorotrityl-chloride resin. Protecting groups may be added or removed in accordance with standard techniques, which are well-known to those skilled in the art and as described herein. It will also be appreciated by those skilled in the art, although such protected derivatives of compounds described herein may not possess pharmacological activity as such, they may be administered to a subject and thereafter metabolized in the body to form compounds described herein which are pharmacologically active. Such derivatives may therefore be described as “prodrugs”. All prodrugs of compounds described herein are included within the scope of the use described herein.

As used herein, the term “prodrug” means a form of an instant compound (e.g., a drug precursor) that is transformed in vivo to yield an active compound of Formula (I) or a form thereof. The transformation may occur by various mechanisms (e.g., by metabolic and/or non-metabolic chemical processes), such as, for example, by hydrolysis and/or metabolism in blood, liver and/or other organs and tissues. A discussion of the use of prodrugs is provided by T. Higuchi and W. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987.

In one example, when a compound of Formula (I) or a form thereof contains a carboxylic acid functional group, a prodrug can comprise an ester formed by the replacement of the hydrogen atom of the acid group with a functional group such as alkyl and the like. In another example, when a compound of Formula (I) or a form thereof contains a hydroxyl functional group, a prodrug form can be prepared by replacing the hydrogen atom of the hydroxyl with another functional group such as alkyl, alkylcarbonyl or a phosphonate ester and the like. In another example, when a compound of Formula (I) or a form thereof contains an amine functional group, a prodrug form can be prepared by replacing one or more amine hydrogen atoms with a functional group such as alkyl or substituted carbonyl. Pharmaceutically acceptable prodrugs of compounds of Formula (I) or a form thereof include those compounds substituted with one or more of the following groups: carboxylic acid esters, sulfonate esters, amino acid esters, phosphonate esters and mono-, di- or triphosphate esters or alkyl substituents, where appropriate. As described herein, it is understood by a person of ordinary skill in the art that one or more of such substituents may be used to provide a compound of Formula (I) or a form thereof as a prodrug.

One or more compounds described herein may exist in unsolvated as well as solvated forms with pharmaceutically acceptable solvents such as water, ethanol, and the like, and the description herein is intended to embrace both solvated and unsolvated forms.

As used herein, the term “solvate” means a physical association of a compound described herein with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. As used herein, “solvate” encompasses both solution-phase and isolatable solvates. Non-limiting examples of suitable solvates include ethanolates, methanolates, and the like.

As used herein, the term “hydrate” means a solvate wherein the solvent molecule is water.

The compounds of Formula (I) can form salts, which are intended to be included within the scope of this description. Reference to a compound of Formula (I) or a form thereof herein is understood to include reference to salt forms thereof, unless otherwise indicated. The term “salt(s)”, as employed herein, denotes acidic salts formed with inorganic and/or organic acids, as well as basic salts formed with inorganic and/or organic bases. In addition, when a compound of Formula (I) or a form thereof contains both a basic moiety, such as, without limitation an amine moiety, and an acidic moiety, such as, but not limited to a carboxylic acid, zwitterions (“inner salts”) may be formed and are included within the term “salt(s)” as used herein.

The term “pharmaceutically acceptable salt(s)”, as used herein, means those salts of compounds described herein that are safe and effective (i.e., physiologically acceptable) for use in mammals and that possess biological activity, although other salts are also useful. Salts of the compounds of the Formula (I) may be formed, for example, by reacting a compound of Formula (I) or a form thereof with an amount of acid or base, such as an equivalent amount, in a medium such as one in which the salt precipitates or in an aqueous medium followed by lyophilization.

Pharmaceutically acceptable salts include one or more salts of acidic or basic groups present in compounds described herein. Particular aspects of acid addition salts include, and are not limited to, acetate, ascorbate, benzoate, benzenesulfonate, bisulfate, bitartrate, borate, bromide, butyrate, chloride, citrate, camphorate, camphorsulfonate, ethanesulfonate, formate, fumarate, gentisinate, gluconate, glucaronate, glutamate, iodide, isonicotinate, lactate, maleate, methanesulfonate, naphthalenesulfonate, nitrate, oxalate, pamoate, pantothenate, phosphate, propionate, saccharate, salicylate, succinate, sulfate, tartrate, thiocyanate, toluenesulfonate (also known as tosylate), trifluoroacetate salts and the like. Certain particular aspects of acid addition salts include chloride, bromide or dichloride.

Additionally, acids which are generally considered suitable for the formation of pharmaceutically useful salts from basic pharmaceutical compounds are discussed, for example, by P. Stahl et al, Camille G. (eds.) Handbook of Pharmaceutical Salts. Properties, Selection and Use. (2002) Zurich: Wiley-VCH; S. Berge et al, Journal of Pharmaceutical Sciences (1977) 66(1) 1-19; P. Gould, International J. of Pharmaceutics (1986) 33, 201-217; Anderson et al, The Practice of Medicinal Chemistry (1996), Academic Press, New York; and in The Orange Book (Food & Drug Administration, Washington, D.C. on their website). These disclosures are incorporated herein by reference thereto.

Suitable basic salts include, but are not limited to, aluminum, ammonium, calcium, lithium, magnesium, potassium, sodium and zinc salts.

All such acid salts and base salts are intended to be included within the scope of pharmaceutically acceptable salts as described herein. In addition, all such acid and base salts are considered equivalent to the free forms of the corresponding compounds for purposes of this description.

Compounds of Formula (I) and forms thereof, may further exist in a tautomeric form. All such tautomeric forms are contemplated and intended to be included within the scope of the compounds of Formula (I) or a form thereof as described herein.

The compounds of Formula (I) or a form thereof may contain asymmetric or chiral centers, and, therefore, exist in different stereoisomeric forms. The present description is intended to include all stereoisomeric forms of the compounds of Formula (I) as well as mixtures thereof, including racemic mixtures.

The compounds described herein may include one or more chiral centers, and as such may exist as racemic mixtures (R/S) or as substantially pure enantiomers and diastereomers. The compounds may also exist as substantially pure (R) or (S) enantiomers (when one chiral center is present). In one particular aspect, the compounds described herein are (S) isomers and may exist as enantiomerically pure compositions substantially comprising only the (S) isomer. In another particular aspect, the compounds described herein are (R) isomers and may exist as enantiomerically pure compositions substantially comprising only the (R) isomer. As one of skill in the art will recognize, when more than one chiral center is present, the compounds described herein may also exist as a (R,R), (R,S), (S,R) or (S,S) isomer, as defined by IUPAC Nomenclature Recommendations.

As used herein, the term “substantially pure” refers to compounds consisting substantially of a single isomer in an amount greater than or equal to 90%, in an amount greater than or equal to 92%, in an amount greater than or equal to 95%, in an amount greater than or equal to 98%, in an amount greater than or equal to 99%, or in an amount equal to 100% of the single isomer.

In one aspect of the description, a compound of Formula (I) or a form thereof that is a substantially pure single isomer may exhibit stronger desired activity than the other substantially pure isomer of the compound of Formula (I) or the racemic mixture thereof.

In one aspect of the description, a compound of Formula (I) or a form thereof is a substantially pure (S) enantiomer form present in an amount greater than or equal to 90%, in an amount greater than or equal to 92%, in an amount greater than or equal to 95%, in an amount greater than or equal to 98%, in an amount greater than or equal to 99%, or in an amount equal to 100%.

In one aspect of the description, a compound of Formula (I) or a form thereof is a substantially pure (R) enantiomer form present in an amount greater than or equal to 90%, in an amount greater than or equal to 92%, in an amount greater than or equal to 95%, in an amount greater than or equal to 98%, in an amount greater than or equal to 99%, or in an amount equal to 100%.

As used herein, a “racemate” is any mixture of isometric forms that are not “enantiomerically pure”, including mixtures such as, without limitation, in a ratio of about 50/50, about 60/40, about 70/30, or about 80/20.

In addition, the present description embraces all geometric and positional isomers. For example, if a compound of Formula (I) or a form thereof incorporates a double bond or a fused ring, both the cis- and trans-forms, as well as mixtures, are embraced within the scope of the description. Diastereomeric mixtures can be separated into their individual diastereomers on the basis of their physical chemical differences by methods well known to those skilled in the art, such as, for example, by chromatography and/or fractional crystallization. Enantiomers can be separated by use of chiral HPLC column or other chromatographic methods known to those skilled in the art. Enantiomers can also be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereomers to the corresponding pure enantiomers. Also, some of the compounds of Formula (I) may be atropisomers (e.g., substituted biaryls) and are considered as part of this description.

All stereoisomers (for example, geometric isomers, optical isomers and the like) of the present compounds (including those of the salts, solvates, esters and prodrugs of the compounds as well as the salts, solvates and esters of the prodrugs), such as those which may exist due to asymmetric carbons on various substituents, including enantiomeric forms (which may exist even in the absence of asymmetric carbons), rotameric forms, atropisomers, and diastereomeric forms, are contemplated within the scope of this description, as are positional isomers (such as, for example, 4-pyridyl and 3-pyridyl). Individual stereoisomers of the compounds described herein may, for example, be substantially free of other isomers, or may be present in a racemic mixture, as described supra.

The term “isotopologue” refers to isotopically-enriched compounds described herein which are identical to those recited herein, but for the fact that one or more atoms are replaced by an atom having an atomic mass or mass number different from the atomic mass or mass number usually found in nature. Examples of isotopes that can be incorporated into compounds described herein include isotopes of hydrogen, carbon, nitrogen, oxygen, phosphorus, fluorine and chlorine, such as ²H, ³H, ¹³C, ¹⁴C, ¹⁵N, ¹⁸O, ¹⁸O, ¹⁷O, ³¹P, ³²P, ³⁵S, ¹⁸F, ³⁵Cl and ³⁶Cl, respectively, each of which are also within the scope of this description.

Certain isotopically-enriched compounds described herein (e.g., those labeled with ³H and ¹⁴C) are useful in compound and/or substrate tissue distribution assays. Tritiated (i.e., ³H) and carbon-14 (i.e., ¹⁴C) isotopes are particularly preferred for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances.

Polymorphic crystalline and amorphous forms of the compounds of Formula (I) and of the salts, solvates, hydrates, esters and prodrugs of the compounds of Formula (I) are further intended to be included in the present description.

### Compound Uses

An aspect of the present description relates to a method of use of a compound of Formula (I) or a form thereof for treating or ameliorating a disorder or condition by inhibiting dihydroorotate dehydrogenase (DHODH) in a subject in need thereof, comprising administering an effective amount of the compound of Formula (I), or a form thereof, to the subject. Another aspect provided herein is a method of treating a disease or disorder amenable to dihydroorotate dehydrogenase inhibition using a compound of Formula (I), or a form thereof, or pharmaceutical composition thereof. Another aspect provided herein is a method of treating a disease or disorder amenable to dihydroorotate dehydrogenase inhibition in a subject in need thereof comprising administering to the subject an effective amount of a compound of Formula (I), or a form thereof, or pharmaceutical composition thereof.

In addition to use as a monotherapy, the instant compounds are useful in a combination therapy with current standard of agents, having additive or synergistic activity with one or more known agents. A combination therapy comprising compounds described herein in combination with one or more known drugs may be used to treat such disorders regardless of whether the disorder is responsive to the known drug.

Certain aspects of the present description include the use of a compound of Formula (I) or a form thereof in a combination therapy for treating or the disorder or condition in a subject in need thereof, comprising administering an effective amount of the compound of Formula (I) or a form thereof and an effective amount of one or more agent(s).

As used herein, the term “treating” refers to preventing a disease, disorder or condition from occurring in a subject that may be predisposed to the disease, disorder and/or condition but has not yet been diagnosed as having the disease, disorder and/or condition.

As used herein, the term “ameliorating” refers to inhibiting a disease, disorder or condition, i.e., arresting the development thereof; and/or relieving a disease, disorder or condition, i.e., causing regression of the disease, disorder and/or condition.

As used herein, the term “subject” refers to an animal or any living organism having sensation and the power of voluntary movement, and which requires oxygen and organic food. In certain aspects, the subject is a mammal or a warm-blooded vertebrate animal. In other aspects, the subject is a human. As used herein, the term “patient” may be used interchangeably with “subject” and “human”.

As used herein, the terms “effective amount” or “therapeutically effective amount” mean an amount of compound of Formula (I) or a form, composition or medicament thereof that achieves a target plasma concentration that is effective in treating or ameliorating the disease or condition at issue as described herein and thus producing the desired therapeutic, ameliorative, inhibitory or preventative effect in a subject in need thereof. In one aspect, the effective amount may be the amount required to treat the disorder or condition in a subject or patient, more specifically, in a human.

### Dosage and Administration

One aspect provided herein is a use of a compound of Formula (I), or a form thereof, or pharmaceutical composition thereof, to treat or ameliorate a disease or disorder by inhibiting dihydroorotate dehydrogenase.

Another aspect provided herein is a use of a compound of Formula (I), or a form thereof, to treat or ameliorate a disease or disorder by inhibiting dihydroorotate dehydrogenase.

Another aspect provided herein is a use of a pharmaceutical composition comprising a compound of Formula (I), or a form thereof, and a pharmaceutically acceptable excipient to treat or ameliorate a disease or disorder by inhibiting dihydroorotate dehydrogenase.

One aspect provided herein is a method of use of a compound of Formula (I), or a form thereof, or pharmaceutical composition thereof to treat or ameliorate a disease or disorder in a subject in need thereof comprising, administering to the subject an effective amount of the compound of Formula (I), or a form thereof, or pharmaceutical composition thereof to inhibit dihydroorotate dehydrogenase.

Another aspect provided herein is a method of use of a compound of Formula (I), or a form thereof, to treat or ameliorate a disease or disorder in a subject in need thereof comprising, administering to the subject an effective amount of a compound of Formula (I), or a form thereof, to inhibit dihydroorotate dehydrogenase.

Another aspect provided herein is a method of use of a pharmaceutical composition comprising a compound of Formula (I), or a form thereof, and a pharmaceutically acceptable excipient to treat or ameliorate a disease or disorder in a subject in need thereof comprising, administering to the subject an effective amount of the pharmaceutical composition to inhibit dihydroorotate dehydrogenase.

In one aspect, the methods of use of a compound of Formula (I), or a form thereof, to treat or ameliorate a disease or disorder in a subject in need thereof comprising, administering to the subject an effective amount of a compound of Formula (I), or a form thereof, to inhibit dihydroorotate dehydrogenase, the compound of Formula (I), or a form or pharmaceutical composition thereof may be administered to a subject in need thereof by a variety of routes in amounts which result in a beneficial or therapeutic effect.

In one aspect, routes of administration include, but are not limited to, oral, intravenous, intradermal, intrathecal, intramuscular, subcutaneous, intranasal, inhalation, transdermal, topical, transmucosal, intracranial, epidural and intra-synovial.

In another aspect, the compound of Formula (I), or a form or pharmaceutical composition thereof may be orally administered to a subject in need thereof in an effective amount of a compound of Formula (I), or a form thereof, to inhibit dihydroorotate dehydrogenase, as provided herein.

In another aspect, the compound of Formula (I), or a form or pharmaceutical composition thereof may be administered orally, with or without food or water.

In another aspect, the compound of Formula (I), or a form or pharmaceutical composition thereof may be administered systemically (e.g., parenterally) to a subject in need thereof.

In another aspect, the compound of Formula (I), or a form or pharmaceutical composition thereof may be administered via a route that permits the compound of Formula (I), or a form or pharmaceutical composition thereof to cross the blood-brain barrier (e.g., orally).

In another aspect, the compound of Formula (I), or a form or pharmaceutical composition thereof may be administered in combination with one or more additional therapies that may be administered by the same route or a different route of administration.

In another aspect, the dosage and frequency of administration of the compound of Formula (I), or a form or pharmaceutical composition thereof in an effective amount to inhibit dihydroorotate dehydrogenase to treat or ameliorate a disease or disorder in a subject in need thereof can be determined by a practitioner, in light of factors related to the subject that requires treatment while minimizing any side effects.

Factors which may be taken into account include the severity of the disease state, general health of the subject, age, weight, and gender of the subject, diet, time and frequency of administration, drug combination(s), reaction sensitivities, and tolerance/response to therapy.

The dosage and frequency of administration of the compound of Formula (I), or a form or pharmaceutical composition thereof may be adjusted over time to provide an effective amount of the compound of Formula (I), or a form or pharmaceutical composition thereof to maintain a desired effect.

In one aspect, the term “effective amount” refers to that amount of the compound of Formula (I), or a form or pharmaceutical composition thereof administered as a monotherapy to a patient, which effective amount is in a range of from about 0.001 mg/Kg/day to about 500 mg/Kg/day, or about 0.01 mg/Kg/day to about 500 mg/Kg/day, or about 0.1 mg to about 500 mg/Kg/day, or about 1.0 mg/day to about 500 mg/Kg/day, in single, divided, or a continuous dose for a patient or subject having a weight in a range of between about 40 to about 200 Kg (which dose may be adjusted for patients or subjects above or below this range, particularly children under 40 Kg). Dosing may be administered as a dose per kilogram, a dose per meter squared or a flat dose expressed in a unit of weight (e.g., milligrams, grams).

In another aspect, the effective amount is a dose administered to the subject that may be increased or decreased depending on subject response. The effective amount for the subject will also depend upon various factors, including the body weight, size and health of the subject. The typical adult subject is expected to have a median weight in a range of between about 60 to about 100 Kg. Accordingly, an effective amount for a given patient may be determined according to the skill and judgment of a practitioner skilled in the art.

In one aspect, daily monotherapy doses may be adjusted based upon the weight of the subject or patient, wherein the compound of Formula (I), or a form or pharmaceutical composition thereof may be formulated for delivery as a monotherapy at about 0.02, 0.025, 0.03, 0.05, 0.06, 0.075, 0.08, 0.09, 0.10, 0.15, 0.20, 0.25, 0.30, 0.35, 0.40, 0.45, 0.50, 0.55, 0.60, 0.65, 0.75, 0.80, 0.90, 1.0, 1.10, 1.20, 1.25, 1.50, 1.75, 2.0, 2.5, 3.0, 3.5, 4.0, 4.5, 5.0, 5.5, 10, 20, 50, 75 or 100 mg/Kg/day or any range in between.

In another aspect, a daily dose may be adjusted based upon the weight of the subject or patient and administered as a single, divided, or continuous dose.

In another aspect, a daily dose of the compound of Formula (I), or a form or pharmaceutical composition thereof may be administered more than once per day, as in once, twice, three times, or more per day.

In another aspect, a dose of the compound of Formula (I), or a form or pharmaceutical composition thereof may be administered more than once per week, as in once, twice, three times, or more per week.

In another aspect, the effective amount may be a dose administered to the subject twice per week on different days, wherein the second dose in a week follows the first by three days, and wherein the first dose in a following week follows the second dose in a preceding week by four days. In another embodiment, a subject may be administered one or more doses of an effective amount of the compound of Formula (I), or a form or pharmaceutical composition thereof, wherein the effective amount may not be the same for each dose.

In one aspect, an effective amount of the compound of Formula (I), or a form or pharmaceutical composition thereof may range from about 0.001 mg/Kg/day to about 500 mg/Kg/day. The terms “effective amount” or “therapeutically effective amount” of the compound of Formula (I), or a form or pharmaceutical composition thereof for use in the manufacture of a medicament or in a method to treat or ameliorate a disease or disorder in a subject in need thereof is an amount sufficient to provide therapeutic benefit by inhibiting dihydroorotate dehydrogenase. The therapeutically effective amount of the compound of Formula (I), or a form or pharmaceutical composition thereof is intended to include an amount administered daily, weekly, biweekly selected from an amount in range of from about 0.01 ng to about 3500 mg; from about 0.1 ng to about 3500 mg; from about 0.1 μg to about 3500 mg; from about 0.1 mg to about 3500 mg; from about 1 mg to about 3500 mg; from about 1 mg to about 3000 mg; from about 0.05 mg to about 1500 mg; from about 0.5 mg to about 1500 mg; from about 1 mg to about 1500 mg; from about 5 mg to about 1500 mg; from about 10 mg to about 600 mg; from about 0.5 mg to about 2000 mg; or, from about 5.0 mg to about 1500 mg.

In one aspect, the effective amount of the compound of Formula (I), or a form or pharmaceutical composition thereof can be estimated initially by results from cell culture assays or from human or relevant animal models, such as the mouse, chimpanzee, marmoset or tamarin animal model. Relevant animal models may also be used to determine the appropriate concentration range and route of administration. Therapeutic efficacy and toxicity may be determined by standard pharmaceutical procedures in cell cultures or experimental animals, e.g., ED₅₀ (the dose therapeutically effective in 50% of the population) and LD₅₀ (the dose lethal to 50% of the population). The dose ratio between the toxic and therapeutic effect is referred to as the therapeutic index, and can be expressed as the ratio, LD₅₀/ED₅₀. In another aspect, the effective amount is such that a large therapeutic index is achieved. In another aspect, the dose administered results in a range of plasma concentrations that include an ED₅₀ with little or no toxicity. The dosage may vary within this range depending upon the dosage form employed, sensitivity of the patient, and the route of administration.

More specifically, the concentration-biological effect (pharmacodynamic) relationship observed with regard to the compound of Formula (I), or a form or pharmaceutical composition thereof suggests a target plasma concentration ranging from about 0.001 μg/mL to about 50 μg/mL, from about 0.01 μg/mL to about 20 μg/mL, from about 0.05 μg/mL to about 10 μg/mL, or from about 0.1 μg/mL to about 5 μg/mL. To achieve such plasma concentrations, the compound of Formula (I), or a form or pharmaceutical composition thereof may be administered at doses that vary from 0.001 μg to 100,000 mg, depending upon the route of administration in single, divided, or continuous doses for a patient weighing between about 40 to about 100 kg (which dose may be adjusted for patients above or below this weight range, particularly for children under 40 kg).

In another aspect, a method for preventing, treating or ameliorating a disease or disorder in a subject in need thereof by inhibiting dihydroorotate dehydrogenase comprises the administration of an effective amount of the compound of Formula (I), or a form or pharmaceutical composition thereof to the subject, wherein the effective amount is a dose selected from a dose in a range of from about 50 mg to about 400 mg, from about 100 mg to about 200 mg, from about 125 mg to about 175 mg, from about 100 mg to about 300 mg, from about 100 mg to about 400 mg, from about 150 mg to about 200 mg, from about 150 mg to about 300 mg, from about 150 mg to about 400 mg, from about 200 mg to about 300 mg, from about 225 mg to about 275 mg, from about 225 mg to about 300 mg, from about 275 mg to about 300 mg, from about 200 mg to about 225 mg, from about 200 mg to about 275 mg, from about 200 mg to about 400 mg, from about 250 mg to about 300 mg, from about 250 mg to about 400 mg, from about 250 mg to about 350 mg, and the like, administered orally once, twice or three times per week.

In another aspect, a method for preventing, treating or ameliorating a disease or disorder in a subject in need thereof by inhibiting dihydroorotate dehydrogenase comprises the administration of an effective amount of the compound of Formula (I), or a form or pharmaceutical composition thereof to the subject, wherein the effective amount is administered once, twice or three times biweekly.

In another aspect, a method for preventing, treating or ameliorating a disease or disorder in a subject in need thereof by inhibiting dihydroorotate dehydrogenase comprises the administration of an effective amount of the compound of Formula (I), or a form or pharmaceutical composition thereof to the subject, wherein the effective amount is administered once, twice or three times every two weeks.

### Pharmaceutical Compositions

Aspects of the present description include the use of a compound of Formula (I) or a form thereof in a pharmaceutical composition for treating or ameliorating a disorder or condition described herein in a subject in need thereof, comprising administering an effective amount of the compound of Formula (I) or a form thereof in admixture with one or more pharmaceutically acceptable excipient(s).

An aspect of the present description includes the use of a pharmaceutical composition of the compound of Formula (I) or a form thereof in the preparation of a kit comprising the pharmaceutical composition of the compound of Formula (I) or a form thereof and instructions for administering the compound for treating or ameliorating the disease or condition in a subject in need thereof.

As used herein, the term “composition” means a product comprising the specified ingredients in the specified amounts, as well as any product which results, directly or indirectly, from combination of the specified ingredients in the specified amounts.

The pharmaceutical composition may be formulated to achieve a physiologically compatible pH, ranging from about pH 3 to about pH 11. In certain aspects, the pharmaceutical composition is formulated to achieve a pH of from about pH 3 to about pH 7. In other aspects, the pharmaceutical composition is formulated to achieve a pH of from about pH 5 to about pH 8.

The term “pharmaceutically acceptable excipient” refers to an excipient for administration of a pharmaceutical agent, such as the compounds described herein. Pharmaceutically acceptable excipients may be determined in part by the particular composition being administered, as well as by the particular mode of administration and/or dosage form. Nonlimiting examples of pharmaceutically acceptable excipients include carriers, solvents, stabilizers, adjuvants, diluents, etc. Accordingly, there exists a wide variety of suitable formulations of pharmaceutical compositions for the instant compounds described herein (see, e.g., Remington's Pharmaceutical Sciences).

Suitable excipients may be carrier molecules that include large, slowly metabolized macromolecules such as proteins, polysaccharides, polylactic acids, polyglycolic acids, polymeric amino acids, amino acid copolymers, and inactive antibodies. Other exemplary excipients include antioxidants such as ascorbic acid; chelating agents such as EDTA; carbohydrates such as dextrin, hydroxyalkylcellulose, hydroxyalkylmethylcellulose (e.g., hydroxypropylmethylcellulose, also known as HPMC), stearic acid; liquids such as oils, water, saline, glycerol and ethanol; wetting or emulsifying agents; pH buffering substances; and the like. Liposomes are also included within the definition of pharmaceutically acceptable excipients.

The pharmaceutical compositions described herein may be formulated in any form suitable for the intended use described herein. Suitable formulations for oral administration include solids, liquid solutions, emulsions and suspensions, while suitable inhalable formulations for pulmonary administration include liquids and powders. Alternative formulations include syrups, creams, ointments, tablets, and lyophilized solids which can be reconstituted with a physiologically compatible solvent prior to administration.

When intended for oral use for example, tablets, troches, lozenges, aqueous or oil suspensions, non-aqueous solutions, dispersible powders or granules (including micronized particles or nanoparticles), emulsions, hard or soft capsules, syrups or elixirs may be prepared. Compositions intended for oral use may be prepared according to any method known to the art for the manufacture of pharmaceutical compositions, and such compositions may contain one or more agents including sweetening agents, flavoring agents, coloring agents, and preserving agents, in order to provide a palatable preparation.

Pharmaceutically acceptable excipients suitable for use in conjunction with tablets include, for example, inert diluents, such as celluloses, calcium or sodium carbonate, lactose, calcium or sodium phosphate; disintegrating agents, such as croscarmellose sodium, cross-linked povidone, maize starch, or alginic acid; binding agents, such as povidone, starch, gelatin or acacia; and lubricating agents, such as magnesium stearate, stearic acid, or talc. Tablets may be uncoated or may be coated by known techniques including microencapsulation to delay disintegration and adsorption in the gastrointestinal tract and thereby provide a sustained action over a longer period. For example, a time delay material such as glyceryl monostearate or glyceryl distearate alone or with a wax may be employed.

Formulations for oral use may be also presented as hard gelatin capsules where the active ingredient is mixed with an inert solid diluent, for example celluloses, lactose, calcium phosphate, or kaolin, or as soft gelatin capsules wherein the active ingredient is mixed with non-aqueous or oil medium, such as glycerin, propylene glycol, polyethylene glycol, peanut oil, liquid paraffin, or olive oil.

In other aspects, pharmaceutical compositions described herein may be formulated as suspensions comprising a compound of Formula (I) or a form thereof in admixture with one or more pharmaceutically acceptable excipient(s) suitable for the manufacture of a suspension. In yet other aspects, pharmaceutical compositions described herein may be formulated as dispersible powders and granules suitable for preparation of a suspension by the addition of one or more excipient(s).

Excipients suitable for use in connection with suspensions include suspending agents, such as sodium carboxymethylcellulose, methylcellulose, hydroxypropyl methylcelluose, sodium alginate, polyvinylpyrrolidone, gum tragacanth, gum acacia, dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethyleneoxycethanol), a condensation product of ethylene oxide with a partial ester derived from a fatty acid and a hexitol anhydride (e.g., polyoxyethylene sorbitan monooleate); and thickening agents, such as carbomer, beeswax, hard paraffin, or cetyl alcohol. The suspensions may also contain one or more preservatives such as acetic acid, methyl and/or n-propyl p-hydroxy-benzoate; one or more coloring agents; one or more flavoring agents; and one or more sweetening agents such as sucrose or saccharin.

The pharmaceutical compositions described herein may also be in the form of oil-in-water emulsions. The oily phase may be a vegetable oil, such as olive oil or arachis oil, a mineral oil, such as liquid paraffin, or a mixture of these. Suitable emulsifying agents include naturally-occurring gums, such as gum acacia and gum tragacanth; naturally occurring phosphatides, such as soybean lecithin, esters or partial esters derived from fatty acids; hexitol anhydrides, such as sorbitan monooleate; and condensation products of these partial esters with ethylene oxide, such as polyoxyethylene sorbitan monooleate. The emulsion may also contain sweetening and flavoring agents. Syrups and elixirs may be formulated with sweetening agents, such as glycerol, sorbitol or sucrose. Such formulations may also contain a demulcent, a preservative, a flavoring or a coloring agent.

Additionally, the pharmaceutical compositions described herein may be in the form of a sterile injectable preparation, such as a sterile injectable aqueous emulsion or oleaginous suspension. Such emulsion or suspension may be formulated according to the known art using those suitable dispersing or wetting agents and suspending agents which have been mentioned above. The sterile injectable preparation may also be a sterile injectable solution or suspension in a parenterally acceptable diluent or solvent, such as a solution in 1,2-propanediol. The sterile injectable preparation may also be prepared as a lyophilized powder. Among the acceptable vehicles and solvents that may be employed are water, Ringer's solution and isotonic sodium chloride solution. In addition, sterile fixed oils may be employed as a solvent or suspending medium. For this purpose any bland fixed oil may be employed including synthetic mono- or di-glycerides. In addition, fatty acids such as oleic acid may likewise be used in the preparation of injectables.

The compounds described herein may be substantially insoluble in water and sparingly soluble in most pharmaceutically acceptable protic solvents and vegetable oils, but generally soluble in medium-chain fatty acids (e.g., caprylic and capric acids) or triglycerides and in propylene glycol esters of medium-chain fatty acids. Thus, contemplated in the description are compounds which have been modified by substitutions or additions of chemical or biochemical moieties which make them more suitable for delivery (e.g., increase solubility, bioactivity, palatability, decrease adverse reactions, etc.), for example by esterification, glycosylation, PEGylation, etc.

In certain aspects, the compound described herein is formulated for oral administration in a lipid-based composition suitable for low solubility compounds. Lipid-based formulations can generally enhance the oral bioavailability of such compounds. As such, pharmaceutical compositions described herein may comprise a effective amount of a compound of Formula (I) or a form thereof, together with at least one pharmaceutically acceptable excipient selected from medium chain fatty acids or propylene glycol esters thereof (e.g., propylene glycol esters of edible fatty acids such as caprylic and capric fatty acids) and pharmaceutically acceptable surfactants, such as polysorbate 20 or 80 (also referred to as Tween® 20 or Tween® 80, respectively) or polyoxyl 40 hydrogenated castor oil.

In other aspects, the bioavailability of low solubility compounds may be enhanced using particle size optimization techniques including the preparation of nanoparticles or nanosuspensions using techniques known to those skilled in the art. The compound forms present in such preparations include amorphous, partially amorphous, partially crystalline or crystalline forms.

In alternative aspects, the pharmaceutical composition may further comprise one or more aqueous solubility enhancer(s), such as a cyclodextrin. Nonlimiting examples of cyclodextrin include hydroxypropyl, hydroxyethyl, glucosyl, maltosyl and maltotriosyl derivatives of α-, β-, and γ-cyclodextrin, and hydroxypropyl-β-cyclodextrin (HPBC). In certain aspects, the pharmaceutical composition further comprises HPBC in a range of from about 0.1% to about 20%, from about 1% to about 15%, or from about 2.5% to about 100%. The amount of solubility enhancer employed may depend on the amount of the compound in the composition.

### General Synthetic Methods of Preparing Compounds

As disclosed herein, general methods for preparing the compounds of Formula (I) or a form thereof as described herein are available via standard, well-known synthetic methodology. Many of the starting materials are commercially available or, when not available, can be prepared using the routes described below using techniques known to those skilled in the art. The synthetic schemes provided herein comprise multiple reaction steps, each of which is intended to stand on its own and can be carried out with or without any preceding or succeeding step(s). In other words, each of the individual reaction steps of the synthetic schemes provided herein in isolation is contemplated.

In the general reaction schemes below, R¹, R², R³, R⁴, R⁵, R⁶, R⁷, R⁸, R⁹, R¹⁰, R¹¹, Q¹, Q², Q³ and Q⁴ have the same meaning as described in the first aspect of Formula (I), above, except as otherwise specified below.

### Scheme A:

Compounds of Formula (I) may be prepared as described in Scheme A below:

Racemic tetrahydro-β-carbolines A3 can be prepared via a Pictet-Spengler reaction between NH₂-containing tryptamines A1 and aldehydes A2 using acid catalyst (AcOH, TFA, TsOH, HCl, etc.) in a solvent (water, alcohol, dichloromethane, AcOH, etc.), as shown below:

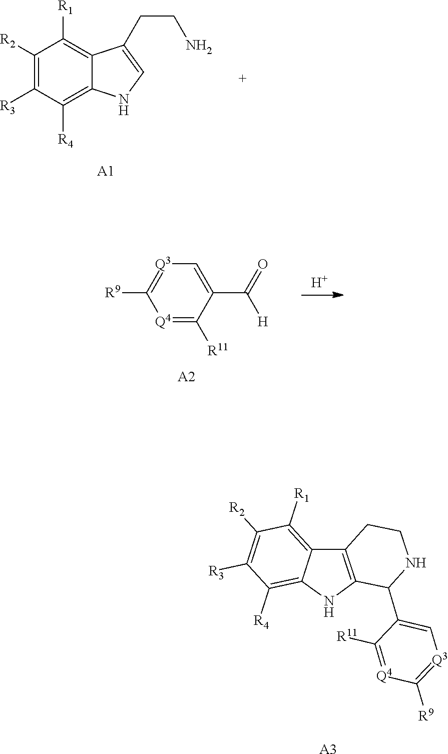

Alternatively, an amide A4 can cyclize under Bischler-Napieralsky reaction conditions (such as heating with POCl3, in a solvent, such as ACN or toluene, or neat) to form the dyhydrocarboline A5, which can be further reduced to racemic tetrahydrocarboline A3 using a reagent such as NaBH4 in an alcoholic solvent, as shown below:

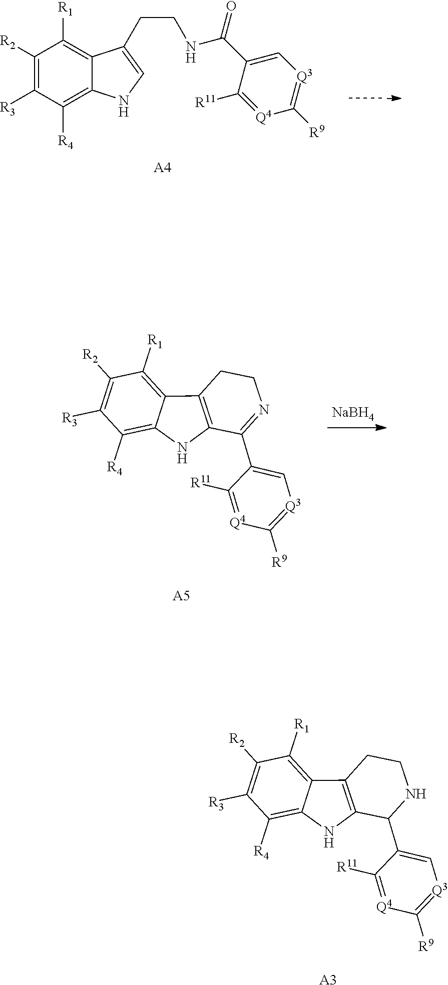

Intermediates A3 can further react (Scheme A) with electrophiles A6 preferably in the presence of a base, such as tertiary amines or alkyl metal carbonates, to form racemic tetrahydro-β-carboline A7, as shown below.

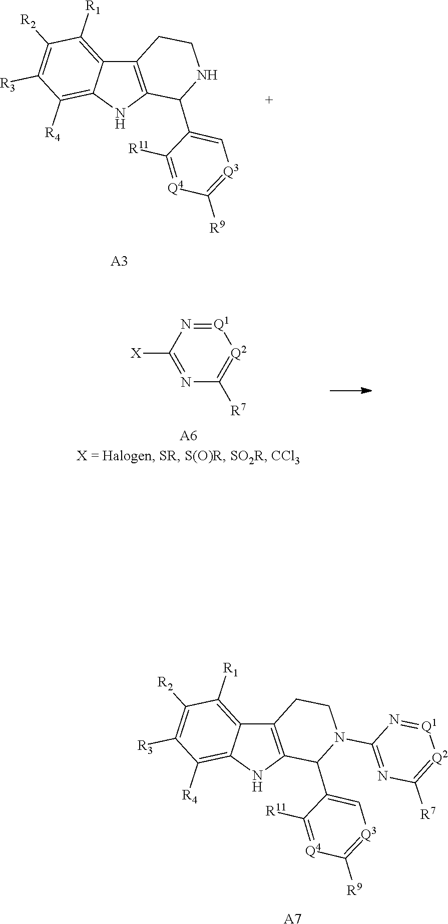

Alternatively, racemic tetrahydro-β-carboline A7 can be prepared from a heteroaryl-containing tryptamine A8 and aldehyde A2 via a Pictet-Spengler reaction using acid catalyst (AcOH, TFA, TsOH, HCl, etc.) in a solvent (water, alcohol, dichloromethane, AcOH, etc.). The tryptamine A8 can be prepared by a reaction between tryptamine A1 and electrophile A6, as shown below:

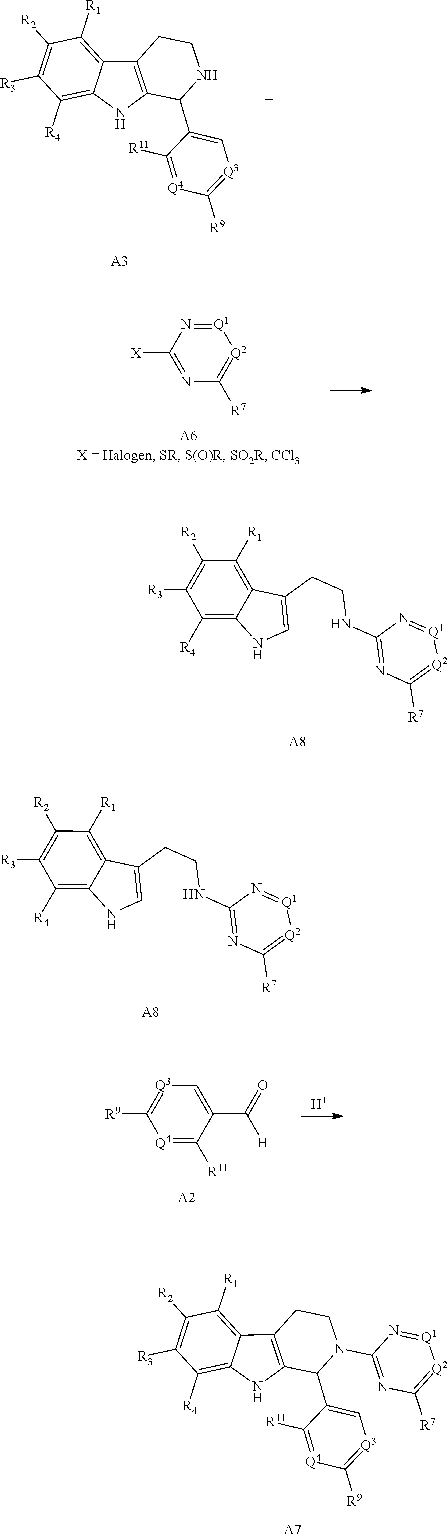

Enantiomeric enrichment of the products of Scheme A may obtained using a variety of methods, including but not limited to asymmetric catalysis, chiral resolution, chiral chromatography and the like, as further shown below.

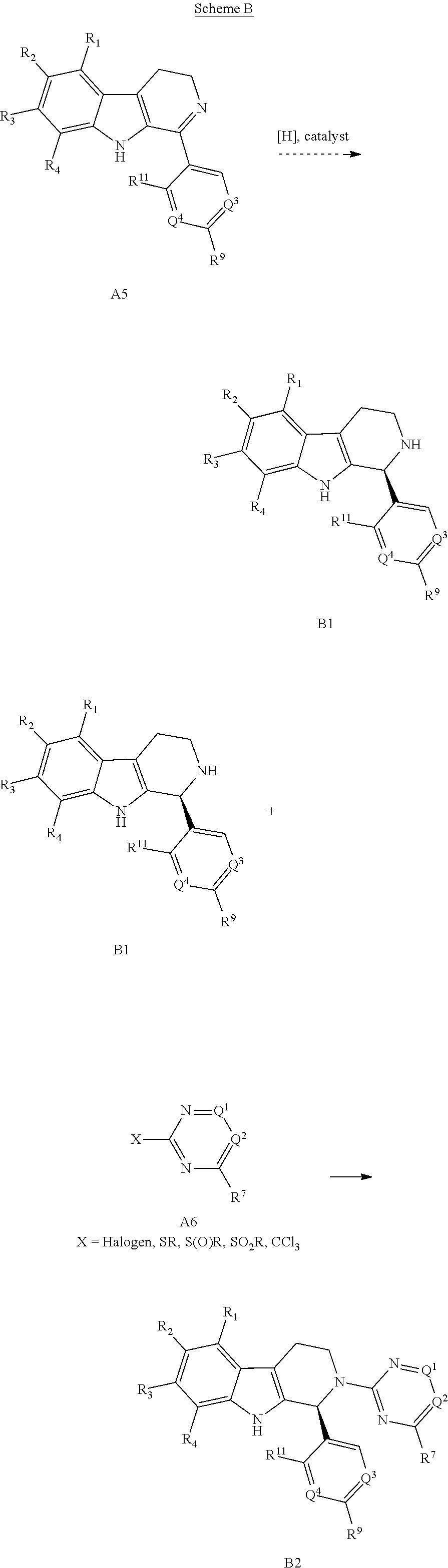

Alternatively (Scheme B), dihydro-β-carboline A5 can be reduced to chiral tetrahydro-β-carboline B1 via asymmetric transfer hydrogenation reaction developed by Ryoji Noyori (see J. Am. Chem. Soc. 1996, 118, 49164917) or similar methods, such as asymmetric hydrogenation using hydrogen-gas. Combining B1 with A6 provides enantio-enriched tetrahydro-β-carboline B2.

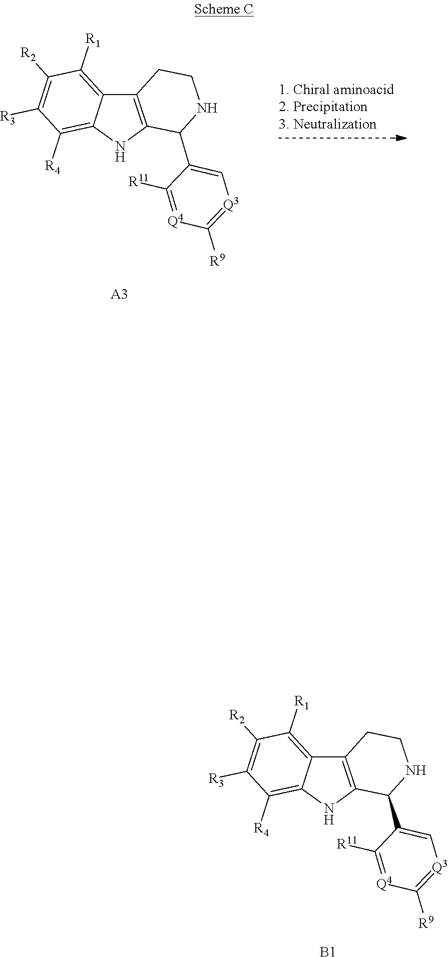

Alternatively (Scheme C), a racemic mixture of A3 can be converted into the enantioenriched B1 via a chiral resolution procedure described in U.S. Pat. No. 7,601,840 B2. In this process, one enantiomer is precipitated selectively as a diastereomeric salt with a chiral amino acid derivative, followed by conversion of this salt into free amine after treatment with a base, such as aqueous ammonia or sodium hydroxide, etc.

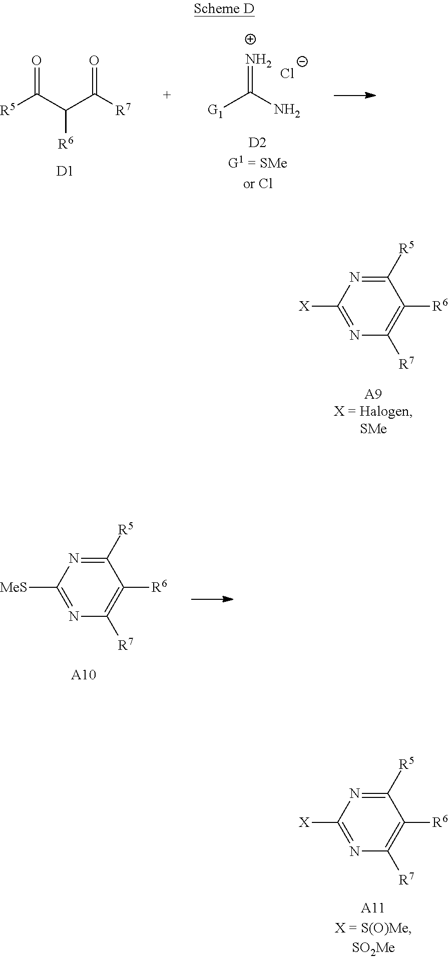

Pyrimidine building blocks A9 (X=Halogen or SMe) can be prepared through a condensation between a dicarbonyl D1 and functionalized amidine D2, optionally catalyzed by acid (H₂SO₄, etc) or in the presence of a dehydrating reagent, such as POCl₃. Thiomethyl analog A10 can additionally be activated further through oxidation to sulfoxide or sulfone A11 using reagents such as 3-chlorobenzenecarboperoxoic acid, etc.

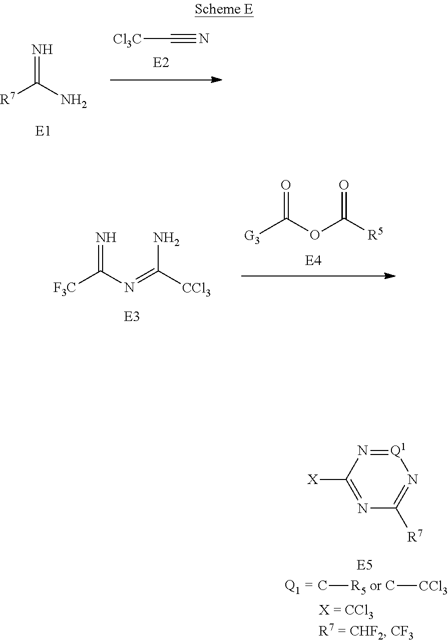

Triazine building blocks E5 (Scheme E, Q¹=CR⁵, Q²=N) can be prepared through a reaction between trifluoromethyl amidine E1 and trichloromethyl acetonitrile E2, followed by condensation between the intermediate E3, which is not isolated, and an acid anhydride E4.

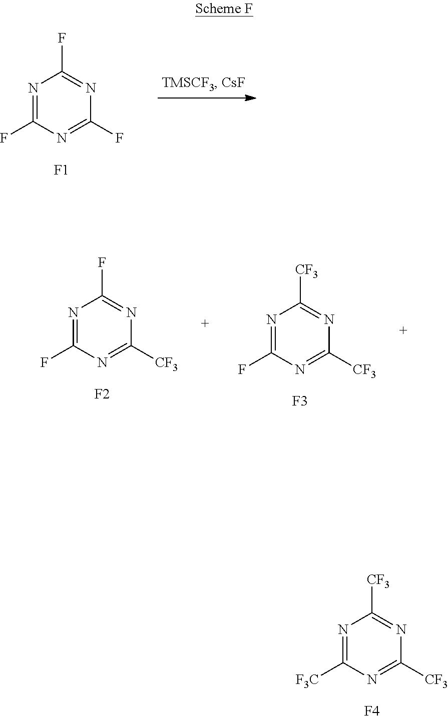

Triazine building blocks F2 and F3 can be formed unselectively through partial displacement of F-atoms in 1,3,5-trifluorotriazine with CF3, Scheme F.

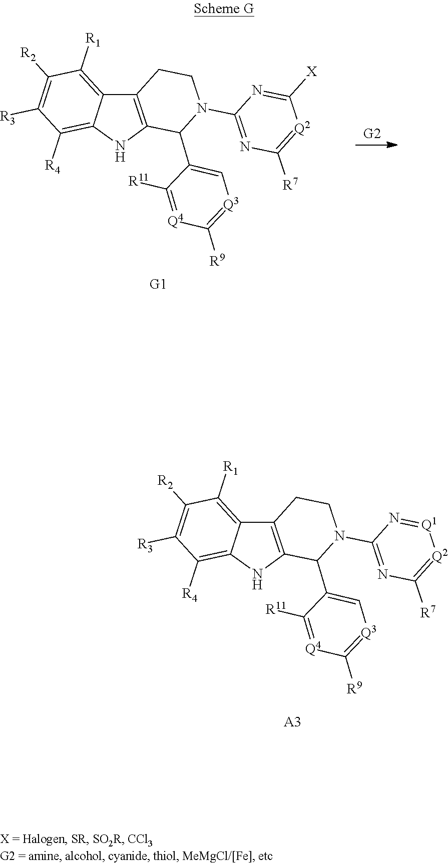

An appropriate leaving group X in G1 (Scheme G) can be replaced by an appropriate nucleophile G2. Alternatively, a thiol group X in G1 can be replaced by H-atom through hydrogenolysis.

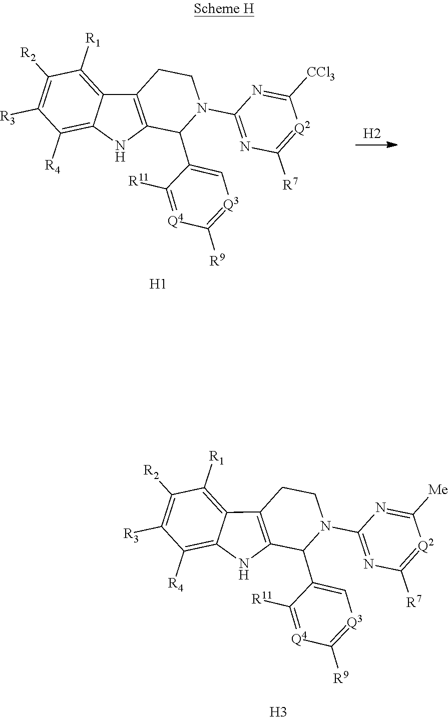

A trichloromethyl group in H1 can be converted into methyl group through reductive hydrogenolysis using metals dissolving in acids, for example, Zn in aqueous ammonium chloride, Scheme H.

### EXAMPLES

The following examples include non-limiting, representative illustrations of aspects of the compounds of Formula (I) described herein. The examples include non-limiting methods for preparing specific compounds of Formula (I).

### SPECIFIC SYNTHETIC EXAMPLES

To assist in understanding the scope of the compounds of Formula (I) or a form thereof described herein, the following general and specific synthetic examples are included. Among other things, these examples illustrate the preparation of certain representative compounds. Those of skill in the art will understand that the techniques described in these examples represent techniques, as described by those of ordinary skill in the art, that function well in synthetic practice, and as such constitute preferred modes for the practice thereof. However, it should be appreciated that those of skill in the art should, in light of the present disclosure, appreciate that many changes can be made in the specific methods that are disclosed and still obtain a like or similar result without departing from the spirit and scope of the present description.

Other than in the following examples, unless indicated to the contrary, all numbers expressing quantities of ingredients, reaction conditions, experimental data, and so forth used in the specification and claims are to be understood as being modified by the term “about”. Accordingly, all such numbers represent approximations that may vary depending upon the desired properties sought to be obtained by a reaction or as a result of variable experimental conditions. Therefore, within an expected range of experimental reproducibility, the term “about” in the context of the resulting data, refers to a range for data provided that may vary according to a standard deviation from the mean. As well, for experimental results provided, the resulting data may be rounded up or down to present data consistently, without loss of significant figures.

While the numerical ranges and parameters setting forth the characterization of the compounds of Formula (I) or a form thereof described herein are approximations, the numerical values set forth in the working examples are reported as precisely as possible. Any numerical value, however, inherently contains certain errors necessarily resulting from the standard deviation found in their respective testing measurements.

The reagents and solvents were used as purchased (from a variety of vendors), except where noted. Where applicable, the term “Celite” is used as shown in the following examples to represent the tradename CELITE® (brand of diatomaceous earth). Where applicable, chromatographic separations were performed using techniques and equipment commonly available such as, for example, by using an ISCO CombiFlash® Rf system. Where applicable, NMR spectra were obtained using techniques and equipment commonly available such as, for example, by using a Bruker Avance III⁵⁰⁰ spectrometer with deuterated solvents such as, for example, DMSO-d₆ or residual solvent as standard. Where applicable, melting points were determined using techniques and equipment commonly available such as, for example, by using a SRS OptiMelt® MPA100 (values as obtained without correction/calibration). Where applicable, TLC analysis was performed using techniques and equipment commonly available such as, for example, by using Aldrich 254 nm glass-backed plates (60 Å, 250 μm), visualized using UV and I₂ stains. Where applicable, ESI mass spectra were obtained using techniques and equipment commonly available such as, for example, by using an ACQUITY UPLC® System, with values shown as [M+H]⁺ or [M−H]⁻, unless otherwise indicated. Where applicable, the structure of the product was obtained via a 2D NOESY (Nuclear Overhauser SpectroscopY) experiment.

The following abbreviations are provided to ensure the terms used herein are unambiguous to one skilled in the art:

| Abbreviation | Meaning |
|---|---|
| AcOH or HOAc | acetic acid |
| ACN or MeCN | acetonitrile |
| AlMe₃ | trimethylaluminum |
| APC | allylpalladium (II) chloride dimer |
| aq. | aqueous |
| Boc | tert-butoxycarbonyl |
| CsOAc | cesium acetate |
| 18-Crown-6 | 1,4,7,10,13,16-hexaoxacyclooctadecane |
| DCM or CH₂Cl₂ | dichloromethane |
| DIBAL-H | diisobutylaluminium hydride |
| DME | dimethoxyethane |
| DMF | dimethyl formamide |
| DMA | dimethylacetamide |
| DMAP | 4-dimethylaminopyridine |
| DMSO | dimethylsulfoxide |
| eq.,or equiv. | eguivalent |
| EtOAc | ethyl acetate |
| EtOH | ethanol |
| HPLC | high performance liquid chromatography |
| h, hr, min, s | hour (h or hr), minute (min), second (s) |
| iPrMgCl*LiCl | isopropylmagnesium chloride lithium chloride complex |
| iPrOAc | isopropyl acetate |
| K₂CO₃ | potassium carbonate |
| K₃PO₄ | potassium phosphate |
| KOtBu or t-BuOK | potassium tert-butoxide |
| LC/MS, LCMS or LC-MS | liquid chromatographic mass spectroscopy |
| MeOH | methanol |
| MeNH₂ × HCl | methylamine hydrochloride |
| MS | mass spectroscopy |
| m.p. | melting point (shown in ° Centigrade) |
| MPS | potassium peroxymonosulfate (2KHSO₅ · KHSO₄ · K₂SO₄) |
| NaH | sodium hydride |
| NaHCO₃ | sodium bicarbonate |
| NaHMDS | sodium hexamethyldisilazide |
| NaIO₄ | sodium periodate |
| NaOH | sodium hydroxide |
| NaOtAm | sodium tert-pentoxide |
| NaOMe | sodium methoxide |
| NaOEt | sodium ethoxide |
| NaOtBu | sodium tert-butoxide |
| NCS | N-chlorosuccinimide |
| NH₄Cl | ammonium chloride |
| NH₄OH | ammonium hydroxide |
| NIS | N-iodosuccinimide |
| NMP | N-methylpyrrolidone |
| NMR | nuclear magnetic resonance |
| Oxone | potassium peroxymonosulfate |
| PCl₅ | phosphorus perchloride or phosphorus pentachloride |
| PCy₃ | tricyclohexylphosphine |
| [Pd] | palladium |
| Pd/C° | palladium on carbon |
| Pd₂(dba)₃ or Pd₂dba₃ | tris(dibenzylideneacetone)dipalladium(0) |
| Pd(dppf)Cl₂ | [1,1′-bis(diphenylphosphino)ferrocene] dichloropalladium(II) |
| PdCl₂(ACN) | bis(acetonitrile)dichloropalladium(II) |
| PdCl₂(allyl) | chloroallylpalladium(II) dimer |
| [Pd(OAc)₂]₃ | palladium (II) acetate |
| Pd(PPh₃)₄ | tetrakis(triphenylphosphine)palladium |
| PE | petroleum ether |
| POCl₃ | phosphorus oxychloride |
| PPh₃ | triphenylphosphine |
| psi | pounds per square inch pressure |
| Pt/C | platinum on carbon |
| PTSA | p-toluenesulfonic acid |
| Q-Phos or QPhos | 1,2,3,4,5-pentaphenyl-1′-(di-tert-butylphosphino)ferrocene |
| RT | room temperature |
| RBF | round bottom flask |
| RuPhos | dicyclohexyl-[2-[2,6-di(propan-2- |
| | yloxy)phenyl]phenyl]phosphane |
| RuCl[(R,R)- | [N-[(1R,2R)-2-(amino-κN)-1,2-diphenylethyl]-4- |
| TsDPEN](mesitylene) | methylbenzenesulfonamidato-κN]chloro[(1,2,3,4,5,6-η)-1,3,5- |
| | trimethylbenzene]-ruthenium |
| RuCl(p-cymene)[(R,R)- | [N-[(1R,2R)-2-(amino-κN)-1,2-diphenylethyl]-4- |
| Ts-DPEN] | methylbenzenesulfonamidato-κN]chloro[(1,2,3,4,5,6-η)-1- |
| | methyl-4-(1-methylethyl)benzene]-ruthenium |
| TBSO or OTBS | tert-butyldimethylsilyloxy |
| TCDI | 1,1′-thiocarbonyldiimidazole |
| t-Bu | tert-butyl |
| TEA, NEt₃, Et₃N | triethylamine |
| TFA | trifluoroacetic acid |
| TFAA | trifluoroacetic anhydride |
| THF | tetrahydrofuran |
| TsOH X H₂O | p-toluenesulfonic acid monohydrate |
| UPLC | Ultra Performance Liquid Chromatography |
| Xphos or XPhos | 2-dicyclohexylphosphino-2′,4′,6′-triisopropylbiphenyl |
| XPhos Pd G3 | (2-dicyclohexylphosphino-2′,4′,6′-triisopropyl-1,1′-biphenyl)[2- |
| | (2′-amino-1,l′-biphenyl)]palladium(II) methanesulfonate |

### Example 1

### Synthesis of 2-(trichloromethyl)-4,6-bis(trifluoromethyl)-1,3,5-triazine

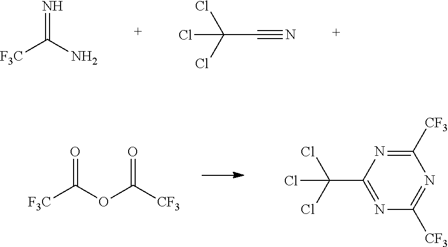 [SMILES: N=C(N)C(F)(F)F ; N#CC(Cl)(Cl)Cl ; O=C(OC(=O)C(F)(F)F)C(F)(F)F ; FC(F)(F)c1nc(C(F)(F)F)nc(C(Cl)(Cl)Cl)n1]

In a round bottomed flask trifluoroacetamidine (50 g, 379.29 mmol) was dissolved in dichloromethane (190 mL). The solution was placed in an ice-water bath at 0° C. and internal temperature was monitored with a thermocouple probe. Trichloroacetonitrile (77 mL, 768 mmol) was added slowly by syringe. After the addition was complete and exothermic reaction seized, the mixture was allowed to stir for further 10 minutes in the ice-water bath. Trifluoroacetic anhydride (64 mL, 455.2 mmol) was then added slowly by syringe. After being allowed to stir for 1H at 0° C., the mixture was poured into saturated sodium bicarbonate (250 mL) pre-chilled in an ice-water bath, and stirred vigorously for 10 minutes. The biphasic mixture was transferred to a separatory funnel and the layers were separated. The aqueous layer was extracted with dichloromethane (2×200 mL) and the combined organic layers were dried over Na₂SO₄, filtered, and concentrated in vacuo. The resulting oil was purified by distillation at 20 mbar from 60° C. and 65° C. (measured at the still head) to give 2-(trichloromethyl)-4,6-bis(trifluoromethyl)-1,3,5-triazine (51.8 g, 41% yield) as a clear oil. ¹⁹F NMR (DMSO-d₆, 400 MHz) δ: −70.96 (s).

### Example 2

### Synthesis of 2-(trichloromethyl)-4-(trifluoromethyl)-1,3,5-triazine

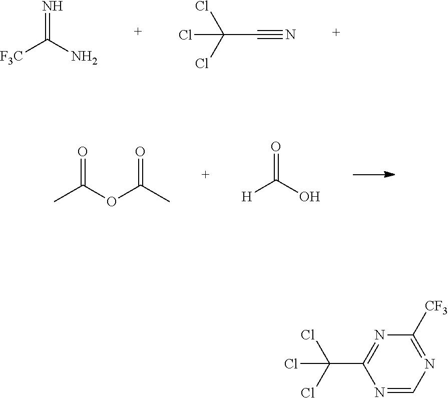

In a flask, formic acid (19 mL, 504 mmol) and acetic anhydride (42 mL, 446 mmol) were combined at 0° C., this mixture was warmed to RT and stirred for 2 Hours. Meanwhile, a separate RBF was flushed with argon and to it was added DCM (90 mL) followed by 2,2,2-trichloroacetonitrile (36 mL, 359 mmol). The solution was then placed in a cooling bath at 0° C. in an ice bath and 2,2,2-trifluoroacetamidine (20 g, 178.49 mmol) was added dropwise at 0° C. After 5 minutes at 0° C. the reaction was complete. The reaction mixture was then placed in a cooling bath at −78° C. and the formic acid/acetic anhydride mixture was added drop-wise by syringe. The mixture was stirred for 5 min at −78° C. then allowed to warm to ambient temperature and stirred for 16 h. The reaction was then poured into a solution of saturated sodium bicarbonate and ice (500 mL) and solid sodium bicarbonate (35 g) was added until bubbling subsided and pH reached 6-7 by pH paper. The aqueous layer was then extracted 3 times with DCM. The combined organic layers were dried over MgSO₄, concentrated on rotovap (down to 110 mbar, ° C.). The resulting oil was purified by distillation at 11 mbar from 65° C. and 85° C. (measured at the still head) to give 2-(trichloromethyl)-4-(trifluoromethyl)-1,3,5-triazine (25 g, 53% yield) as a clear oil. ¹H NMR (DMSO-d₆, 400 MHz) δ: 9.96 (s, 1H). ¹⁹F NMR (DMSO-d₆, 400 MHz) δ: −71.00 (s)

### Example 3

### Synthesis of 2-chloro-4,6-bis(trifluoromethyl)pyrimidine

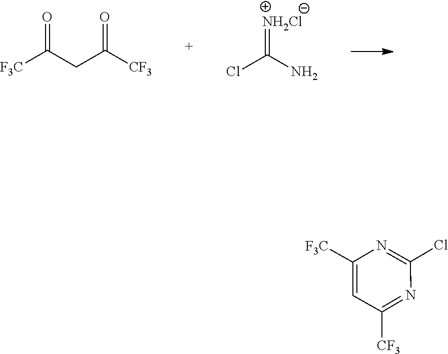

To a 100-mL round-bottom flask equipped with a magnetic stir bar and a reflux condenser was added chloroformamidine hydrochloride (7.26 g, 62.5 mmol) and acetonitrile (20 mL). The mixture was stirred to suspension at RT and hexafluoroacetylacetone (6.79 mL, 48.0 mmol) was added, followed by concentrated sulfuric acid (0.05 mL, 0.9 mmol, 0.02 equiv) and phosphoryl chloride (4.47 mL, 48.0 mmol). The resulting mixture was heated to reflux. After 2H the reaction mixture was cooled to RT and transferred to a distillation apparatus. Distillation at 100 mbar, after a forerun of acetonitrile at just above RT, afforded 2-chloro-4,6-bis(trifluoromethyl)pyrimidine 7.2 g as a clear, colorless oil, 60% yield; b.p. 68° C. at 100 mbar. ¹H NMR (CDCl₃, 400 MHz) 7.94 (s, 1H). ¹⁹F NMR (CDCl₃, 400 MHz) δ 69.68 (s)

### Example 4

### Synthesis of 2-(methylsulfonyl)-4,6-bis(trifluoromethyl)pyrimidine

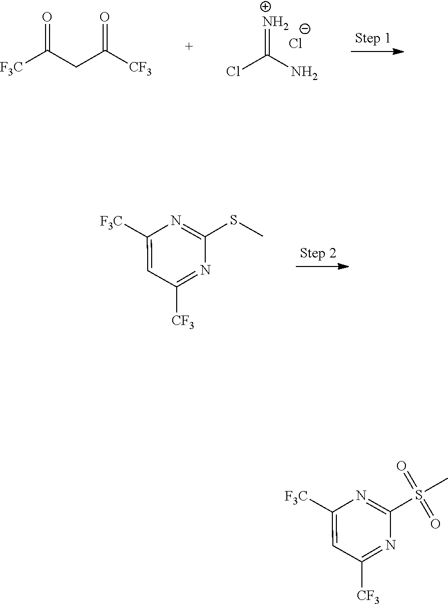

Step 1: To a stirred suspension of 2-methylisothiourea (1.08 g, 12.0 mmol) in ethanol (10 mL, 172 mmol), was added hexafluoroacetylacetone (2.08 g, 10.0 mmol) and sulfuric acid (50 mg, 0.51 mmol) The suspended mixture was heated to reflux for 16 h, and the heat was removed and allowed to reach RT. The reaction was concentrated in vacuo to yield 2-methylsulfanyl-4,6-bis(trifluoromethyl)pyrimidine (2.2 g, 8.4 mmol, 84% yield) without further purification and used by the next step.

Step 2: To a stirred suspension of 2-methylsulfanyl-4,6-bis(trifluoromethyl)pyrimidine (2.2 g, 8.4 mmol) in dichloromethane (25 mL, 390.0 mmol) was added 3-chlorobenzenecarboperoxoic acid (4.3 g, 25 mmol), The suspended mixture was heated to 45° C. and stir for 2 Hrs. The reaction was then cooled back to RT and concentrated in vacuo. The residue was purified by flash chromatography to yield 2-methylsulfonyl-4,6-bis(trifluoromethyl)pyrimidine 1.8 g as a clear oil 73% yield. MS m/z 292.9 [M−H]⁻; (CDCl₃, 400 MHz) d: 8.18 (s, 1H), 3.48 (s, 3H). ¹⁹F NMR (CDCl₃, 400 MHz) δ: −70.25 (s).

### Example 5

### Synthesis of 2-morpholinopyrimidine-5-carboxylic acid

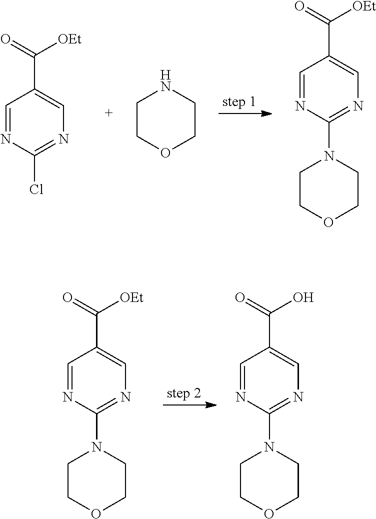 [SMILES: CCOC(=O)c1cnc(Cl)nc1 ; C1COCCN1 ; CCOC(=O)c1cnc(N2CCOCC2)nc1 ; CCOC(=O)c1cnc(N2CCOCC2)nc1 ; O=C(O)c1cnc(N2CCOCC2)nc1]

Step 1: Ethyl 2-chloropyrimidine-5-carboxylate (150 g, 803.9 mmol) was placed in a 3 L 3-neck RBF equipped with a N2 inlet, and dissolved in acetonitrile (1.5 L). Morpholine (74 g, 849.4 mmol) and potassium carbonate (333 g, 2.41 mol) were then added to the solution. The mixture was refluxed for 6 h, then cooled to RT and concentrated in vacuo. The resulting residue was suspended in water (1.5 L) and extracted with ethyl acetate (3×1.5 L). The organic phase was dried over Na₂SO₄ and filtered. The filtrate was concentrated to give ethyl 2-morpholinopyrimidine-5-carboxylate (180 g, 758.7 mmol, 94.4% yield) as a pale yellow solid. The material was used in the next step without further purification. MS m/z 238.0 [M+H]⁺.

Step 2: Ethyl 2-morpholinopyrimidine-5-carboxylate (118 g, 497.3 mmol) was dissolved in a mixture of THF (100 mL) and water (500 mL), then lithium hydroxide (36 g, 1.47 mol) was added. The mixture was stirred at 60° C. for 3 h. The mixture was then cooled to RT and concentrated in vacuo to remove THF. The pH of the aqueous solution was then adjusted to 3-4 with 6N HCl causing organic solids to precipitate. The material was collected by filtration yielding 2-morpholinopyrimidine-5-carboxylic acid (100 g, 478.0 mmol, 96% yield) as a white solid. MS m/z 210.0 [M+H]⁺. ¹H NMR (DMSO): δ 12.83 (s, 1H), 8.78 (s, 2H), 3.87-3.76 (m, 4H), 3.74-3.60 (m, 4H).

### Example 6A

### General Procedure A for Asymmetric Carboline Synthesis

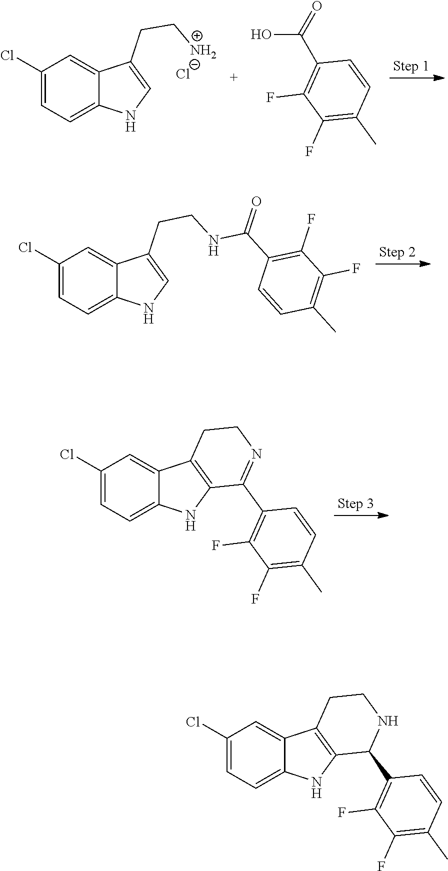 [SMILES: [NH3+]CCc1c[nH]c2ccc(Cl)cc12 ; [Cl-] ; Cc1ccc(C(=O)O)c(F)c1F ; Cc1ccc(C(=O)NCCc2c[nH]c3ccc(Cl)cc23)c(F)c1F ; Cc1ccc(C2=NCCc3c2[nH]c2ccc(Cl)cc32)c(F)c1F ; Cc1ccc([C@@H]2NCCc3c2[nH]c2ccc(Cl)cc32)c(F)c1F]

Step 1: A round bottom flask was charged with 2,3-difluoro-4-methyl-benzoic acid (50 g, 290.48 mmol), 2-(5-chloro-1H-indol-3-yl)ethan-1-amine hydrochloride (73 g, 318.62 mmol), N,N-diisopropylamine (100 mL, 600 mmol) and N,N-dimethylformamide (500 mL), followed by addition of HATU (124 g, 319.6 mmol) in a single portion. The reaction was allowed to stir at ambient temperature for 6 h, at which point the reaction showed consumption of starting material and the desired product was observed by LCMS. The reaction mixture was poured into a separatory funnel and partitioned between EtOAc and water. The organic phase was washed with water (2×1000 mL), then brine (lx 800 mL). The organic layer was dried over Na₂SO₄, filtered and evaporated to dryness. The resulting solid was slurried in acetonitrile, filtered and volatiles removed under reduced pressure and the residue subjected to purification by silica gel chromatography (MeOH/DCM 0-10%) to give N-[2-(5-chloro-1H-indol-3-yl)ethyl]-2,3-difluoro-4-methyl-benzamide (92.5 g, 265 mmol, 91.3% yield) as a yellow solid.

Step 2. In a round bottom flask N-[2-(5-chloro-1H-indol-3-yl)ethyl]-2,3-difluoro-4-methyl-benzamide (46 g, 131.9 mmol) was dissolved in acetonitrile (500 mL). The vessel was flushed with nitrogen and the reaction continued under a nitrogen atmosphere. Phosphoryl chloride (123 mL, 1320 mmol) was added and the reaction mixture was allowed to stir at 90° C. for 6 h at which point LCMS analysis indicated consumption of starting material and formation of the desired product. The reaction mixture was concentrated in vacuo and the oily residue was poured into a separatory funnel and partitioned between EtOAc and saturated aq. bicarbonate. The aqueous layer was extracted with EtOAc (2×1000 mL). The combined organic phases were concentrated in vacuo to give 6-chloro-1-(2,3-difluoro-4-methyl-phenyl)-4,9-dihydro-3H-pyrido[3,4-b]indole (B, 36 g, 108.8 mmol, 82.5% yield) as a yellow solid.

Step 3: A round bottom flask equipped with a stir bar was charged with 6-chloro-1-(2,3-difluoro-4-methyl-phenyl)-4,9-dihydro-3H-pyrido[3,4-b]indole (36 g, 108.8 mmol) and acetonitrile (1000 mL). RuCl(p-cymene)[(R,R)-Ts-DPEN] (4.24 g, 6.53 mmol) and formic acid triethylamine complex 5:2 (93 mL, 218.1 mmol) were added, the reaction vessel was flushed with argon and the reaction proceeded under under an argon atmosphere. The reaction mixture was allowed to stir at 25° C. for 24 h at which point LCMS analysis indicated consumption of starting material and formation of the desired product. The reaction mixture was poured into a separatory funnel with EtOAc and water. The organic layer was washed with saturated aqueous Na₂CO₃ (2×1000 mL) then brine (1×1000 mL), dried over Na₂SO₄ and concentrated in vacuo. The resulting solid was slurried in acetonitrile, filtered and evaporated to dryness to give (1S)-6-chloro-1-(2,3-difluoro-4-methyl-phenyl)-2,3,4,9-tetrahydro-1H-pyrido[3,4-b]indole (29 g, 87.14 mmol, 80.07% yield) as a white solid.

Step 4: (removal of residual ruthenium): To a RBF was added (1S)-6-chloro-1-(2,3-difluoro-4-methyl-phenyl)-2,3,4,9-tetrahydro-1H-pyrido[3,4-b]indole (58 g, 174.3 mmol), dichloromethane (500 mL), SiliaMetS® Thiol (a metal scavenger silica gel from SILICYCLE, Inc.) (12 g) and the mixture was stirred at 25° C. for 3 h. The SiliaMetS Thiol was then removed via filtration, washed twice with dichloromethane (100 mL). The combined organics were concentrated in vacuo, and the residue was further triturated in EtOAc and PE to afford (1S)-6-chloro-1-(2,3-difluoro-4-methyl-phenyl)-2,3,4,9-tetrahydro-1H-pyrido[3,4-b]indole (55 g, 165.3 mmol, 94.83% yield) as a white solid. MS m/z 333 [M+H]^{+ 1}H NMR (DMSO-d₆, 400 MHz) δ: 10.70 (s, 1H), 7.45 (d, 2.0 Hz, 1H), 7.21 (d, J=8.4 Hz, 1H), 7.02-6.97 (m, 2H), 6.70 (t, J=6.8 Hz, 1H), 5.37 (s, 1H), 3.04-2.99 (m, 1H), 2.96-2.90 (m, 1H), 2.87 (s, 1H), 2.74-2.61 (m, 2H), 1.99 (s, 3H). 98.3% e.e.

### Example 6B

### General Procedure B for Racemic Carboline Synthesis

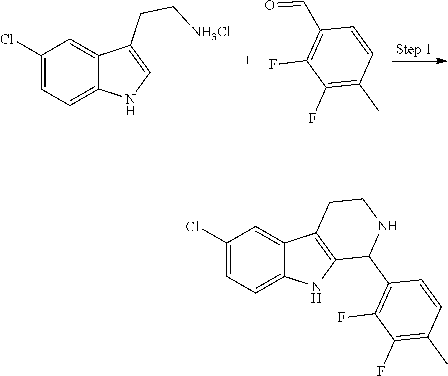

A 100 mL single neck round-bottomed flask was charged with a solution of 2-(5-chloro-1H-indol-3-yl)ethanamine; hydrochloride (1.2 g, 5.2 mmol) in 1,2-dichloroethane (10.0 mL, 126 mmol) followed by 2,3-difluoro-4-methyl-benzaldehyde (1.0 g, 6.4 mmol) and trifluoroacetic acid (1.0 mL, 13 mmol). The mixture was allowed to stir at 90° C. for 3 h. After being allowed to cool to ambient temperature, the mixture was dried under reduced pressure to give a dark residue which was washed with ethyl acetate:petroleum ether=1:10 (2×60.0 mL). The combined organic washing solutions were evaporated to dryness affording 6-chloro-1-(2,3-difluoro-4-methyl-phenyl)-2,3,4,9-tetrahydro-1H-pyrido[3,4-b]indole (1.2 g, 3.6 mmol, 69% yield) without further purification.

### Example 7

### Synthesis of MeO-Pyrimidine Containing Carboline

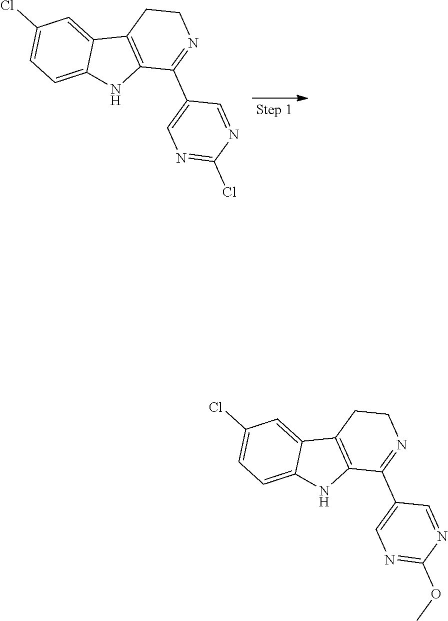 [SMILES: Clc1ccc2[nH]c3c(c2c1)CCN=C3c1cnc(Cl)nc1 ; COc1ncc(C2=NCCc3c2[nH]c2ccc(Cl)cc32)cn1]

Step 1: The starting material 6-chloro-1-(2-chloropyrimidin-5-yl)-4,9-dihydro-3H-pyrido[3,4-b]indole was synthesized from the corresponding tryptamine and carboxylic acid as described in steps 1 and 2 of Example 6A. 6-chloro-1-(2-chloropyrimidin-5-yl)-4,9-dihydro-3H-pyrido[3,4-b]indole (300 mg, 0.95 mmol, 1.0 equiv) was dissolved in methanol (10 mL), then sodium methoxide (850 mg, 15.7 mmol, 16.6 equiv.) was added. The mixture was stirred at RT for one hour. Water was then added to the reaction mixture and the organics were extracted with ethyl acetate 3 times. Combined organic extracts were dried over MgSO₄ and concentrated. The material 6-chloro-1-(2-methoxypyrimidin-5-yl)-4,9-dihydro-3H-pyrido[3,4-b]indole (220 mg, 0.70 mmol, 74% yield) was thus obtained as pale brown solid and used in the asymmetric reduction step described in Step 3 of Example 6A without further purification.

### Example 8

### Synthesis of Compound 233

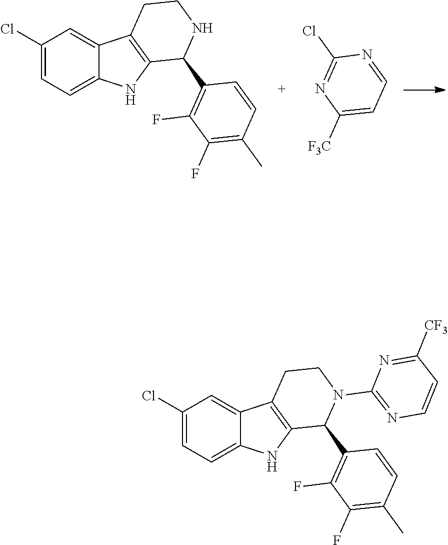 [SMILES: Cc1ccc([C@@H]2NCCc3c2[nH]c2ccc(Cl)cc32)c(F)c1F ; FC(F)(F)c1ccnc(Cl)n1 ; Cc1ccc([C@H]2c3[nH]c4ccc(Cl)cc4c3CCN2c2nccc(C(F)(F)F)n2)c(F)c1F]

To a round bottomed flask containing (1S)-6-chloro-1-(2,3-difluoro-4-methyl-phenyl)-2,3,4,9-tetrahydro-1H-pyrido[3,4-b]indole (25.00 g, 75.12 mmol) was added 1-butanol (200 mL) forming a solution. N,N-diisopropylethylamine (40 mL, 229 mmol), 2-chloro-4-(trifluoromethyl)pyrimidine (18.00 g, 98.61 mmol) were added and the mixture was purged with nitrogen for 5 min. The reaction mixture was allowed to stir at 80° C. for 4 h, then concentrated in vacuo and purified by flash chromatography (petroleum ether:EtOAc gradient 0-10%) to give Compound 233 ((32 g, 82% yield) as a white solid.

MS m/z 479 [M+H]+^{+ 1}H NMR (DMSO-d₆, 400 MHz) δ 11.16 (s, 1H), 8.79 (d, J=4.8 Hz, 1H), 7.54 (d, J=2.0 Hz, 1H), 7.31 (t, J=8.4 Hz, 2H), 7.13 (d, J=4.8 Hz, 1H), 7.09 (q, J=6.4 Hz, 1H), 7.03 (t, J=7.2 Hz, 1H), 6.76 (t, J=6.8 Hz, 1H), 4.90 (d, J=7.2 Hz, 1H), 3.40 (q, J=11.2 Hz, 1H), 2.93-2.83 (m, 2H), 2.60 (d, J=1.6 Hz, 3H). 99% e.e.

Using the procedure described for Compound 233 above, additional compounds described herein were prepared by substituting the appropriate starting materials, suitable reagents and reaction conditions, obtaining compounds such as those selected from:

| Cpd | Data |
|---|---|
| 22 | ¹H NMR (DMSO-d₆) δ: 11.29 (s, 1H), 8.31 (d, J = 4.9 Hz, 1H), 7.65 (d, J = 7.3 Hz, 1H), |
| | 7.29-7.39 (m, 3H), 7.13-7.23 (m, 3H), 6.59 (d, J = 4.9 Hz, 1H), 4.86-4.98 (m, 1H), |
| | 3.07-3.19 (m, 1H), 2.72-2.83 (m, 2H), 2.62 (q, J = 7.3 Hz, 2H), 1.23 (t, J = 7.6 Hz, 3H) |
| 23 | ¹H NMR (DMSO-d₆) δ: 11.29 (s, 1H), 8.32 (d, J = 5.2 Hz, 1H), 7.65 (d, J = 7.3 Hz, 1H), |
| | 7.29-7.39 (m, 3H), 7.12-7.23 (m, 3H), 6.60 (d, J = 4.9 Hz, 1H), 4.88-4.97 (m, 1H), |
| | 3.06-3.22 (m, 1H), 2.74-2.90 (m, 3H), 1.23 (dd, J = 6.7, 3.7 Hz, 6H) |
| 24 | MS m/z 465.1 [M + H]⁺; ¹H NMR (DMSO-d₆) δ: 11.31 (br s, 1H), 8.79 (br d, J = 4.3 |
| | Hz, 1H), 7.68 (d, J = 7.3 Hz, 1H), 7.31-7.38 (m, 3H), 7.20 (app. t, J = 8.9 Hz, 2H), 7.11 |
| | (d, J = 4.9 Hz, 1H), 7.07 (br s, 1H), 6.82-6.83 (m, 1H), 4.78-5.01 (m, 1H), 3.16-3.30 |
| | (m, 1H), 2.75-2.96 (m, 2H) |
| 25 | MS m/z 422.1 [M + H]⁺; ¹H NMR (DMSO-d₆) δ: 11.20-11.40 (m, 1H), 8.75 (br d, |
| | J = 4.6 Hz, 1H), 7.68 (d, J = 7.3 Hz, 1H), 7.31-7.38 (m, 3H), 7.25 (d, J = 4.9 Hz, 1H), |
| | 7.16-7.23 (m, 2H), 7.04 (br s, 1H), 4.73-4.96 (m, 1H), 3.15-3.27 (m, 1H), 2.73-2.95 |
| | (m, 2H) |
| 128 | MS m/z 478.9 [M + H]⁺; ¹H NMR (400 MHz, DMSO-d₆) δ: 11.16 (s, 1H), 8.78 (d, J = |
| | 4.8 Hz, 1H), 7.54 (d, J = 2 Hz, 1H), 7.31 (t, J = 8.8 Hz, 2H), 7.13 (d, J = 4.8 Hz, 1H), |
| | 7.09 (d, J = 2 Hz, 1H), 7.03 (t, J = 7.6 Hz, 1H), 6.76 (t, J = 7.2 Hz, 1H), 4.90 (t, J = |
| | 4.4 Hz, 1H), 3.46 (s, 1H), 2.89 (m, 2H), 2.26 (d, J = 1.2 Hz, 3 H). |
| 161 | MS m/z 443.0 [M + H]⁺; ¹H NMR (400 MHz, DMSO) δ 11.22 (s, 1H), 8.77 (s, 1H), |
| | 7.53 (d, J = 2.0 Hz, 1H), 7.31 (d, J = 8.4 Hz, 1H), 7.18 (dd, J = 8.0, 18.4 Hz, 4H), |
| | 7.11-6.98 (m, 3H), 4.94-4.83 (m, 1H), 3.34-3.24 (m, 1H), 2.91-2.77 (m, 2H), 2.27 (s, 3H). |
| 198 | MS m/z 452 [M − H]⁻; ¹H NMR (400 MHz, DMSO-d₆) δ: 11.43 (s, 1H), 8.76 (d, J = 4.4 |
| | Hz, 1H), 7.29-7.25 (m, 2H), 7.19-7.12 (m, 2H), 7.07-7.03 (m, 1H), 6.80-6.76 (m, |
| | 1H), 4.84-4.81 (m, 1H), 3.41-3.32 (s, 1H), 3.01-3.00 (m, 2H), 2.27 (d, J = 0.8 Hz, 3H). |
| 203 | MS m/z 423 [M − H]⁻; ¹H NMR (400 MHz, DMSO) δ: 11.11 (s, 1H), 8.29 (d, J = 4.0 Hz, |
| | 1H), 7.50 (d, J = 4.0 Hz, 1H), 7.38 (s, 1H), 7.30 (d, J = 8.0 Hz, 1H), 7.07 (dd, J = 8.0, 4.0 |
| | Hz, 1H), 7.01 (t, J = 8.0 Hz, 1H), 6.71 (t, J = 8.0 Hz, 1H), 6.60 (d, J = 4.0 Hz, 1H), 4.88 |
| | (d, J = 12.0 Hz, 1H), 3.34-3.22(m, 1H), 2.80-2.78(m, 2H), 2.32(s, 3H), 2.25(s, 3H). |
| 234 | MS m/z 423.0 [M − H]⁻; ¹H NMR (DMSO-d₆, 400 MHz) δ: 11.12 (s, 1H), 8.29 (d, J = |
| | 4.0 Hz, 1H), 7.51 (d, J = 2.0 Hz, 1H), 7.39 (s, 1H), 7.31 (d, J = 8.0 Hz, 1H), 7.07 (dd, |
| | J = 8.0, 2.0 Hz, 1H), 7.01 (t, J = 4.0 Hz, 1H), 6.72 (t, J = 4.0 Hz, 1H), 6.60 (d, J = |
| | 8.0 Hz, 1H), 4.91-4.87 (m, 1H), 3.35-3.20 (m, 1H), 2.81-2.79 (m, 2H), 2.32 (s, 3H), |
| | 2.25 (d, J = 2.0 Hz, 3H). 98% e.e. |
| 265 | MS m/z 454.3 [M + H]⁺; ¹H NMR (DMSO-d₆) δ: 11.82 (br s, 1H), 8.80 (br s, 1H), |
| | 8.44-8.60 (m, 1H), 8.01 (dd, J = 8.9, 2.1 Hz, 1H), 7.49 (d, J = 8.9 Hz, 1H), 7.16-7.27 (m, |
| | 4H), 7.12 (d, J = 4.9 Hz, 1H), 7.08 (br s, 1H), 4.94 (br s, 1H), 3.26-3.33 (m, 1H), 2.98- |
| | 3.09 (m, 1H), 2.85-2.98 (m, 1H), 2.28 (s, 3H) |
| 385 | MS m/z 454.0 [M + H]⁺; (DMSO-d₆, 400 MHz) 11.20 (s, 1H), 8.35 (d, J = 6.4 Hz, 1H), |
| | 7.56 (d, J = 2.0 Hz, 1H), 7.35 (d, J = 8.8 Hz, 1H), 7.26 (s, 1H), 7.11 (dd, J = 2.0 and J = 8.8 |
| | Hz, 1H), 7.06 (t, J = 7.2 Hz, 1H), 6.66 (t, J = 6.8 Hz, 1H), 4.49 (d, J = 13.6 Hz, 1H), 3.49- |
| | 3.40 (m, 1H), 2.92-2.89 (m, 2H), 2.27 (d, J = 1.6 Hz, 3H). |
| 392 | MS m/z 418.0 [M + H]⁺; (DMSO-d₆, 400 MHz) 11.22 (s, 1H), 8.92 (s, 1H), 7.52(d, |
| | J = 2.0 Hz, 1H), 7.32 (d, J = 8.8 Hz, 1H), 7.22-7.16 (m, 4H), 7.08 (dd, J = 2.0 and J = 8.4 |
| | Hz, 1H), 6.93 (s, 1H), 4.73 (d, J = 10.8 Hz, 1H), 3.30-3.21 (m, 1H), 2.87-2.80 (m, 2H), |
| | 2.28 (s, 3H). |
| 393 | MS m/z 392.9 [M + H]⁺; ¹H NMR (400 MHz, DMSO) δ 11.21 (s, 1H), 8.53 (d, J = 0.8 |
| | Hz, 2H), 7.50 (d, J = 2.0 Hz, 1H), 7.31 (d, J = 8.4 Hz, 1H), 7.13-7.18 (m, 4H), 7.06 |
| | (dd, J = 8.8 Hz, 1H), 7.02 (s, 1H), 4.75-4.79 (m, 1H), 3.15-3.23 (m, 1H), 2.78-2.81 |
| | (m, 2H), 2.26(s, 3H). |
| 394 | MS m/z 406.9 [M + H]⁺; ¹H NMR (400 MHz, DMSO) δ 11.19 (s, 1H), 8.36 (d, J = 1.6 |
| | Hz, 1H), 7.49 (d, J = 2.0 Hz, 1H), 7.30 (d, J = 8.8 Hz, 1H), 7.17 (d, J = 8.0 Hz, 2H), 7.13 |
| | (d, J = 8.4 Hz, 2H), 7.06 (q, J = 2.2 Hz, 1H), 7.03 (s, 1H), 4.78 (d, J = 12.0 z, 1H), 3.15- |
| | 3.18 (m, 1H), 2.77-2.80 (m, 2H), 2.36 (d, J = 2.4 Hz, 3H), 2.26 (s, 3H). |
| 395 | MS m/z 420.9 [M + H]⁺; ¹H NMR (400 MHz, DMSO) δ 11.18 (s, 1H), 7.49 (d, J = 2.0 |
| | Hz, 1H), 7.29 (d, J = 8.4 Hz, 1H), 7.15 (q, J = 8.2 Hz, 4H), 7.05 (q, J = 2.2 Hz, 1H), 4.80 |
| | (d, J = 10.0 Hz, 1H), 3.09-3.16 (m, 1H), 2.75-2.77 (m, 2H), 2.32 (d, J = 2.4 Hz, 6H), |
| | 2.26 (s, 3H). |
| 396 | MS m/z 491.0 [M + H]⁺; ¹H NMR (400 MHz, DMSO) δ 11.10 (s, 1H), 8.93 (s, 1H), |
| | 8.32 (s, 2H), 7.55 (d, J = 2.0 Hz, 1H), 7.32 (d, J = 8.8 Hz, 1H), 7.09 (dd, J = 2.0 and J = 8.4 |
| | Hz, 1H), 6.70 (s, 1H), 4.87-4.82 (m, 1H), 3.67-3.62 (m, 9H), 2.95-2.78 (m, 2H). |
| 398 | MS m/z 498. [M + H]⁺; ¹H NMR (400 MHz, DMSO) δ 8.34 (s, 2H), 8.09 (s, 1H), 7.51 |
| | (d, J = 1.6 Hz, 1H), 7.19 (d, J = 8.8 Hz, 1H), 7.14 (q, J = 1.8 Hz, 1H), 7.05 (s, 1H), 6.83 |
| | (s, 1H), 5.02 (q, J = 6.0 Hz, 1H), 3.80 (t, J = 4.8 Hz, 4H), 3.73 (t, J = 4.6 Hz, 4H), 3.35- |
| | 3.38 (m, 1H), 2.93 (t, J = 3.2 Hz, 2H), |
| 400 | MS m/z 490.0 [M + H]⁺; ¹H NMR (400 MHz, DMSO) δ 11.14 (s, 1H), 8.92 (s, 1H), |
| | 8.10 (d, J = 2.4 Hz, 1H), 7.53 (d, J = 2.0 Hz, 1H), 7.43 (dd, J = 2.4 Hz and J = 8.8 Hz, 1H), |
| | 7.32 (d, J = 8.4 Hz, 1H), 7.08 (dd, J = 2.0 Hz and J = 8.8 Hz, 1H), 6.81-6.79 (m, 2H), |
| | 4.79 (s, 1H), 3.68-3.65 (m, 4H), 3.42-3.38 (m, 5H), 2.92-2.80 (m, 2H). |
| 402 | MS m/z 403.9 [M + H]⁺; ¹H NMR (400 MHz, DMSO) δ 11.22 (s, 1H), 7.52 (d, J = 2.0 |
| | Hz, 1H), 7.31 (d, J = 8.8 Hz, 1H), 7.14-7.18 (m, 5H), 7.08 (dd, J = 8.6 Hz, 1H), 4.92 |
| | (dd, J = 13.2 Hz, 1H), 3.10-3.17 (m, 1H), 2.72-2.87 (m, 2H), 2.38(s, 3H), 2.33(s, 3H), |
| | 2.27(s, 3H). |
| 408 | MS m/z 513.9 [M − H]⁻; ¹H NMR (DMSO-d₆) δ: 11.09 (s, 1H), 8.79 (d, J = 4.8 Hz, 1H), |
| | 8.34 (s, 2H), 7.55 (d, J = 2.0 Hz, 1H), 7.32 (d, J = 8.8 Hz, 1H), 7.07-7.12 (m, 2H), 6.81 |
| | (s, 1H), 4.99-5.01 (m, 1H), 3.61-3.67 (m, 8H), 3.43-3.44 (m, 1H), 2.80-2.96 (m, 2H). |
| 413 | MS m/z 468.0 [M + H]⁺; ¹H NMR (400 MHz, DMSO) δ 11.12 (s, 1H), 7.54 (d, J = 2.0 |
| | Hz, 1H), 7.31 (d, J = 8.8 Hz, 1H), 7.14 (s, 1H), 7.08 (dd, J = 2.0 and J = 8.4 Hz, 1H), |
| | 7.05-7.01 (m, 1H), 6.75 (t, J = 7.2 Hz, 1H), 4.74 (s, 1H), 3.40-3.34 (m, 1H), 2.86-2.81 |
| | (m, 2H), 2.44 (d, J = 2.4 Hz, 3H), 2.26 (d, J = 1.8 Hz, 3H). |
| 417 | MS m/z 432.0 [M + H]⁺; ¹H NMR (400 MHz, DMSO) δ 11.19 (s, 1H), 7.52 (d, J = 2.0 |
| | Hz, 1H), 7.31 (d, J = 8.4 Hz, 1H), 7.20-7.15 (m, 4H), 7.08 (dd, J = 2.0 and J = 8.4 Hz, |
| | 1H), 6.94 (s, 1H), 4.72 (s, 1H), 3.27-3.21 (m, 1H), 2.84-2.79 (m, 2H), 2.46 (s, 3H), |
| | 2.28 (s, 3H). |
| 421 | MS m/z 505.0 [M + H]⁺; ¹H NMR (400 MHz, DMSO) δ 11.08 (s, 1H), 8.33 (s, 2H), |
| | 7.54 (d, J = 1.2 Hz, 1H), 7.31 (d, J = 8.4 Hz, 1H), 7.08 (dd, J = 2.0 and J = 8.4 Hz, 1H), |
| | 6.70 (s, 1H), 4.85 (s, 1H), 3.67-3.62 (m, 8H), 3.34-3.30 (m, 1H), 2.94-2.89 (m, 1H), |
| | 2.82-2.72 (m, 1H), 2.47 (s, 3H). |
| 424 | MS m/z 504.0 [M + H]⁺; ¹H NMR (400 MHz, DMSO) δ 11.15 (s, 1H), 8.11 (d, J = 2.0 |
| | Hz, 1H), 7.52 (d, J = 2.0 Hz, 1H), 7.41 (dd, J = 2.0 and J = 8.8 Hz, 1H), 7.31 (d, J = 8.4 |
| | Hz, 1H), 7.07 (dd, J = 2.0 and J = 8.4 Hz, 1H), 6.80-6.68 (m, 2H), 4.78 (s, 1H), 3.67- |
| | 3.65 (m, 4H), 3.44-3.38 (m, 4H), 3.32-3.25 (m, 1H), 2.90-2.76 (m, 2H), 2.46 (s, 3H). |
| 463 | MS m/z 491.1 [M + H]⁺; ¹H NMR (CDCl₃) δ: 8.33 (s, 1H), 8.00 (m, 1H), 7.48 (m, 1H), |
| | 7.23-7.18 (m, 2H), 7.13-7.10 (s, 1H), 6.85-6.75 (m, 2H), 5.12-5.07(m, 1H), 3.50-3.43 |
| | (m, 1H), 2.93-2.80 (m, 2H), 2.27 (m, 3H), 1H not observed (indole NH) |
| 473 | MS m/z 512.9 [M − H]⁻; ¹H NMR (DMSO-d₄) δ: 11.15 (s, 1H), 8.78 (s, 1H), 8.13 (s, |
| | 1H), 7.54 (s, 1H), 7.44 (d, J = 8.8 Hz, 1H), 7.31 (d, J = 8.4 Hz, 1H), 7.07-7.11 (m, 2H), |
| | 6.90-6.94 (m, 1H), 6.86 (d, J = 8.4 Hz, 1H), 4.95-4.97 (m, 1H), 3.66 (s, 4H), 3.40 (s, |
| | 4H), 3.35 (s, 1H), 2.78-2.93 (m, 2H) |
| 481 | MS m/z 596.2 [M + H]⁺; ¹H NMR (DMSO-d₆) δ: 11.13 (s, 1H), 8.13 (d, J = 2.4 Hz, 1H), |
| | 7.56 (d, J = 1.6 Hz, 1H), 7.53 (s, 1H), 7.40 (q, J = 6.8 Hz, 1H), 7.31 (d, J = 8.8 Hz, 1H), |
| | 7.08 (q, J = 6.8 Hz, 1H), 6.88 (s, 1H), 6.79 (d, J = 8.8 Hz, 1H), 4.92 (q, J = 8.0 Hz, 1H), |
| | 3.46-3.40 (m, 5H), 2.98 (q, J = 12.0 Hz, 1H), 2.88-2.80 (m, 1H), 2.36 (t, J = 4.0 Hz, |
| | 4H), 2.20 (s, 3H) |
| 487 | MS m/z 628.6 [M + H]⁺; ¹H NMR (DMSO-d₆, 500 MHz) § 11.16 (s, 1H), 8.1-8.2 (m, |
| | 1H), 7.71 (s, 1H), 7.55 (s, 1H), 7.44 (dd, 1H, J = 2.3, 9.0 Hz), 7.2-7.3 (m, 1H), 7.2- |
| | 7.2 (m, 1H), 6.89 (s, 1H), 6.81 (d, J = 8.9 Hz, 1H), 4.93 (dd, J = 5.5, 13.4 Hz, 1H), |
| | 3.65 (t, J = 4.7 Hz, 4H), 3.4-3.5 (m, 1H), 3.40 (t, J = 4.7 Hz, 4H), 2.9-3.0 (m, 1H), |
| | 2.8-2.9 (m, 1H) |
| 496 | MS m/z 611.0 [M + H]⁺; ¹H NMR (400 MHz, DMSO) δ 11.11 (s, 1H), 8.35 (s, 2H), |
| | 7.58 (d, J = 1.6 Hz, 1H), 7.56 (s, 1H), 7.33 (d, J = 8.8 Hz, 1H), 7.12-7.09 (m, 1H), 6.78 |
| | (s, 1H), 5.00-4.95 (m, 1H), 3.84-3.80 (m, 2H), 3.72-3.63 (m, 2H), 3.55-3.46 (m, 2H), |
| | 3.05-2.99 (m, 3H), 2.87-2.84 (m, 1H), 1.20 (s, 6H). |
| 518 | MS m/z 590.9 [M + H]⁺; ¹H NMR (DMSO-d₆, 400 MHz) δ: 11.17 (s, 1H), 7.71 (s, |
| | 1H), 7.56 (s, 1H), 7.2-7.3 (m, 1H), 7.2-7.2 (m, 2H), 7.03 (br t, J = 7.5 Hz, 1H), 6.82 |
| | (t, J = 7.1 Hz, 1H), 4.8-5.0 (m, 1H), 3.5-3.6 (m, 1H), 2.8-3.0 (m, 2H), 2.2-2.3 (m, 3H) |

### Example 9

### Synthesis of Compound 412

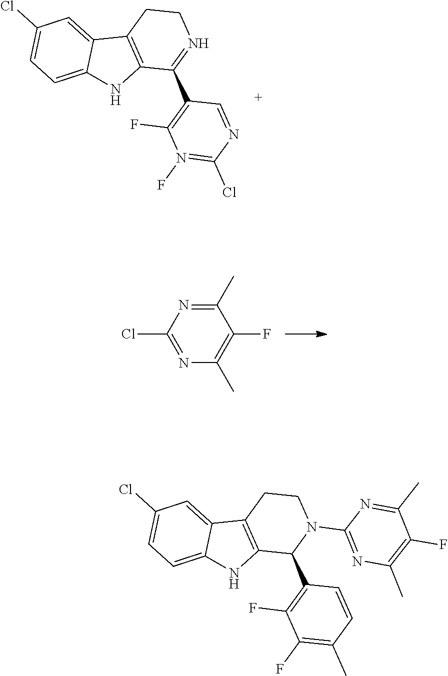

To a mixture of (1S)-6-chloro-1-(2,3-difluoro-4-methyl-phenyl)-2,3,4,9-tetrahydro-1H-pyrido[3,4-b]indole (50 mg, 0.15 mmol) and 2-chloro-5-fluoro-4,6-dimethyl-pyrimidine (36 mg, 0.22 mmol) in NMP (4 mL) was added N,N-diisopropylethylamine (60 mg, 0.46 mmol), and the mixture was stirred at 210° C. for 4 hours in a microwave reactor. The resulting mixture was purified directly via Prep-HPLC to obtain Compound 412 (15 mg, 0.033 mmol, 21.9%/yield) as a white solid. MS m/z 457.1 [M+H]⁺; ¹H NMR (DMSO-d₄) 11.10 (s, 1H), 7.50 (d, J=2.40 Hz, 1H), 7.29 (d, J=8.4 Hz, 1H), 7.24 (s, 1H), 7.06 (q, J=6.4 Hz, 1H), 6.99 (t, J=7.6 Hz, 1H), 6.71 (t, J=6.8 Hz, 1H), 4.79 (q, J=7.2 Hz, 1H), 3.28 (t, 1H), 2.79 (t, =4.8 Hz, 2H), 2.30 (d, J=2.4 Hz, 6H), 2.24 (d, J=0.8 Hz, 3H), 22% e.e. (partial epimerization due to high temperature).

Using the procedure described for Compound 412 above, additional compounds described herein were prepared by substituting the appropriate starting materials, suitable reagents and reaction conditions, obtaining compounds such as those selected from:

| Cpd | Data |
|---|---|
| 238 | MS m/z 428.9 [M + H]+; ¹H NMR (DMSO-d₄) δ: 11.13(s, 1H), 8.55(d, J = 0.4 Hz, |
| | 2H), 7.51 (d, J = 2.0 Hz, 1H), 7.31 (d, J = 8.8 Hz, 1H), 7.29 (s, 1H), 7.07 (q, J = 6.8 Hz, |
| | 1H), 7.02 (t, J = 7.6 Hz, 1H), 6.68 (t, J = 6.8 Hz, 1H), 4.76 (q, J = 7.6 Hz, 1H), 3.33- |
| | 3.26 (m, 1H), 2.80 (t, J = 4.8 Hz, 2H) 2.26 (d, J = 1.2 Hz, 3H). 96% e.e. |
| 364 | MS m/z 466.0 [M + H]+; ¹H NMR (400 MHz, DMSO) δ 11.09 (s, 1H), 8.54 (s, 2H), |
| | 8.13 (s, 2H), 7.53 (d, J = 2.0 Hz, 1H), 7.31 (d, J = 8.4 Hz, 1H), 7.07 (dd, J = 8.6 Hz, |
| | 1H), 6.77 (s, 1H), 4.87-4.91 (m, 1H), 3.61-3.65 (m, 9H), 2.77-2.89 (m, 2H) |
| 397 | MS m/z 423.0 [M + H]+; H NMR (400 MHz, CDCl3) δ 7.72 (s, 1H), 7.52 (d, J = 1.6 |
| | Hz, 1H), 7.27 (s, 1H), 7.18 (d, J = 8.4 Hz, 1H), 7.12-7.15 (m, 3H), 6.99 (s, 1H), 6.94 |
| | (s, 1H), 4.96 (q, J = 3.0 Hz, 1H), 3.41-3.48 (m, 1H), 2.92-2.95 (m, 2H), 2.33 (s, 3H). |
| 401 | MS m/z 429.0 [M + H]+; (DMSO-d, 400 MHz) 11.13 (s, 1H), 8.55 (d, J = 0.4 Hz, 2H), |
| | 7.51 (d, J = 2.0 Hz, 1H), 7.31 (d, J = 8.8 Hz, 1H), 7.29 (s, 1H), 7.07 (q, J = 6.8 Hz, 1H), |
| | 7.02 (t, J = 7.6 Hz, 1H), 6.68 (t, J = 6.8 Hz, 1H), 4.76 (q, J = 7.6 Hz, 1H), 3.33-3.26 (m, |
| | 1H), 2.80 (t, J = 4.8 Hz, 2H) 2.26 (d, J = 1.2 Hz, 3H). |
| 470 | MS m/z 528.0 [M + H]+; (CDCl3, 400 MHz), δ: 8.278(s, 2H), 8.272(s, 2H), 7.79(s, |
| | 1H), 7.45-7.43(m, 1H), 7.12-7.04(m, 2H), 6.81(s, 1H), 4.95-4.91(m, 1H), 3.70- |
| | 3.64(m, 8H), 3.22-3.14(m, 1H), 2.85-2.74(m, 2H), |
| 480 | MS m/z 528.0 [M + H]+; ¹H NMR (CDCl3, 400 MHz), δ 8.54-8.53(d, J = 4.4, 2H), |
| | 8.21(s, 1H), 7.93(s, 1H), 7.50 (m, 2H), 7.18-7.10(dd, J = 8.4, 2H), 6.99(s, 1H), 6.79- |
| | 6.78(d, J = 4.4, 1H), 6.5-6.54(d, J = 8.8, 1H), 5.11-5.06(m, 1H), 3.53(m, 4H), 3.38- |
| | 3.31(m, 1H), 2.98-2.83(m, 2H), 2.49-2.47(m, 4H), 2.32(s, 3H), |
| 484 | MS m/z 529.2 [M + H]+; ¹H NMR (400 MHz, DMSO) δ 11.09 (s, 1H), 8.78 (d, J = 4.0 |
| | Hz, 1H), 8.31 (s, 2H), 7.55 (s, 1H), 7.32 (d, J = 8.4 Hz, 1H), 7.08-7.11 (m, 2H), 6.80 |
| | (s, 1H), 4.99-5.00 (m, 1H), 3.69 (s, 4H), 3.36 (s, 1H), 2.76-2.95 (m, 2H), 2.31 (s, |
| | 4H), 2.18 (s, 3H). |
| 503 | MS m/z 610.9 [M + H]+; ¹H NMR (400 MHz, DMSO) δ 11.08 (s, 1H), 8.29 (s, 2H), |
| | 7.57 (d, J = 1.6 Hz, 1H), 7.54 (s, 1H), 7.32 (d, J = 8.4 Hz, 1H), 7.09 (dd, J = 8.4 Hz, |
| | 1H), 6.74 (s, 1H), 4.97 (dd, J = 13.6 Hz, 1H), 4.47-4.50 (m, 2H), 3.46-3.54 (m, 1H), |
| | 3.00 (dd, J = 15.6 Hz, 1H), 2.79-2.86 (m, 1H), 2.63 (b, 2H), 2.32 (t, J = 11.4 Hz, 2H), |
| | 0.99 (dd, J = 6.2 Hz, 6H). |
| 512 | MS m/z 608.9 [M + H]+; ¹H NMR (400 MHz, DMSO) δ 11.09 (s, 1H), 8.28 (s, 2H), |
| | 7.57 (d, J = 2.0 Hz, 1H), 7.54 (s, 1H), 7.32 (d, J = 8.4 Hz, 1H), 7.09 (dd, J = 8.4 Hz, |
| | 1H), 6.74 (s, 1H), 4.97 (dd, J = 13.4 Hz, 1H), 3.69 (t, J = 5.0 Hz, 2H), 3.56 (s, 2H), |
| | 3.47-3.54 (m, 1H), 2.99 (dd, J = 15.6 Hz, 1H), 2.80-2.86 (m, 1H), 2.77 (t, J = 5.0 Hz, |
| | 2H), 0.44 (s, 4H). |

### Example 10

### Preparation of Compound 283

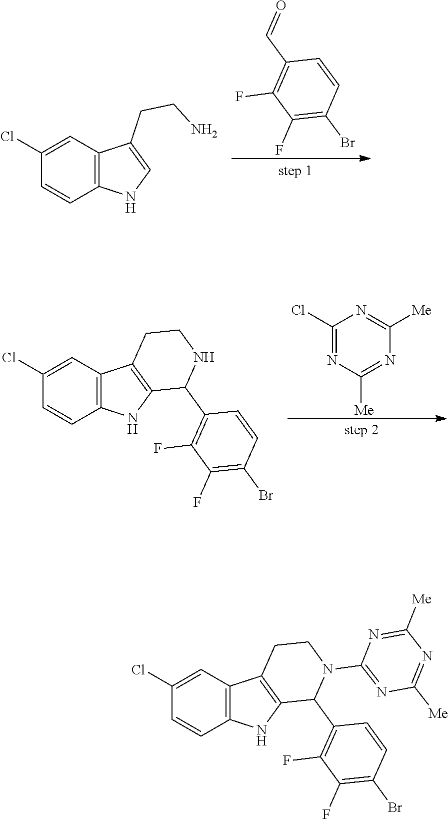 [SMILES: NCCc1c[nH]c2ccc(Cl)cc12 ; O=Cc1ccc(Br)c(F)c1F ; Fc1c(Br)ccc(C2NCCc3c2[nH]c2ccc(Cl)cc32)c1F ; Cc1nc(C)nc(Cl)n1 ; Cc1nc(C)nc(N2CCc3c([nH]c4ccc(Cl)cc34)C2c2ccc(Br)c(F)c2F)n1]

Step 1: Prepared according to general procedure B, 1-(4-bromo-2,3-difluoro-phenyl)-6-chloro-2,3,4,9-tetrahydro-1H-pyrido[3,4-b]indole (500 mg, 1.258 mmol, 81.6% yield) was isolated as a white solid.

Step 2: A 20 mL screw-cap vial was charged with 1-(4-bromo-2,3-difluoro-phenyl)-6-chloro-2,3,4,9-tetrahydro-1H-pyrido[3,4-b]indole (300 mg, 0.7545 mmol), 2-chloro-4,6-dimethyl-1,3,5-triazine (162 mg, 1.1284 mmol) and 1-butanol (5 mL). Then triethylamine (0.316 mL, 2.27 mmol) was added and the resulting mixture was allowed to stir at 100° C. for 2 h. After being allowed to cool to ambient temperature volatiles were removed under reduced pressure and the resulting dark residue was purified subjected to preparatory HPLC purification (water:acetonitrile gradient 5 to 100%). Compound 283 (310 mg, 0.614 mmol, 81.4% yield) was isolated as a white solid.

MS m/z 506.0 [M+H]⁺; ¹H NMR (DMSO-d₆) δ: ppm 11.17 (s, 1H), 7.57 (s, 1H), 7.52-7.46 (m, 1H), 7.37-7.33 (m, 1H), 7.33-7.26 (m, 1H), 7.12 (dd, J=, 8.70, 1.98 Hz, 1H), 6.90 (s, 1H), 5.05-4.95 (m, 1H), 3.33-3.27 (m, 1H), 2.95-2.88 (m, 1H), 2.86-2.76 (m, 1H), 2.37 (s, 6H).

Using the procedure described for Compound 283 above, additional compounds described herein were prepared by substituting the appropriate starting materials, suitable reagents and reaction conditions, obtaining, compounds such as those selected from:

| Cpd | Data |
|---|---|
| 205 | ESI-MS: m/z 457.0 [M + H]⁺; ¹H NMR (400 MHz, DMSO-d₆) δ: 11.38 (s, 1H), |
| | 7.38 (s, 1H), 7.15-7.10 (m, 2H), 7.03-6.99 (m, 1H), 6.80-6.76 (m, 1H), 6.49 (s, |
| | 1H), 4.94-4.89 (m, 1H), 3.30-3.25 (m, 1H), 2.95-2.93 (m, 2H), 2.27-2.25 (m, 9H). |
| 213 | ESI-MS: m/z 491.0 [M − H]⁻; (DMSO, 400 MHz) δ: 11.15 (s, 1H), 7.54 (d, 2.0 Hz, |
| | 1H), 7.32-7.21 (m, 2H), 7.10-7.06 (m, 2H), 7.02 (t, J = 7.2 Hz, 1H), 6.77 (t, |
| | J = 7.2 Hz, 1H), 5.01-4.82 (m, 1H), 3.42-3.34 (m, 1H), 2.92-2.81 (m, 2H), 2.46 |
| | (s, 3H), 2.25 (s, 3H). |
| 253 | ESI-MS: m/z 440.0 [M + H]⁺; ¹H NMR (500 MHz, DMSO-d₆) δ 11.14 (s, 1H), 7.54 |
| | (d, J = 2.0 Hz, 1H), 7.41-7.22 (m, 2H), 7.14-6.93 (m, 2H), 6.77 (t, J = 7.3 Hz, |
| | 1H), 4.97 (dd, J = 13.4, 5.4 Hz, 1H), 3.29 (q, J = 8.4, 6.8 Hz, 1H), 3.06-2.71 (m, |
| | 2H), 2.51 (s, 3H), 2.26 (s, 3H). |
| 264 | MS m/z 487.4 [M + H]⁺; ¹H NMR (DMSO-d₆) δ: 11.22 (br s, 1H), 8.78 (br s, 1H), |
| | 7.67 (s, 1H), 7.28 (d, J = 8.4 Hz, 1H), 7.15-7.22 (m, 5H), 7.10 (d, J = 4.9 Hz, 1H), |
| | 6.98-7.08 (m, 1H), 4.91 (br s, 1H), 3.23-3.31 (m, 1H), 2.79-2.92 (m, 2H), 2.28 (s, 3H) |
| 293 | ESI-MS: m/z 47.2 [M + H]⁺; ¹H NMR (500 MHz, DMSO-d₆) δ 11.02 (s, 1H), 8.26 |
| | (s, 2H), 7.47 (d, J = 2.1 Hz, 1H), 7.25 (d, J = 8.5 Hz, 1H), 7.02 (dd, J = 8.5, 2.1 Hz, |
| | 1H), 6.84 (s, 1H), 4.96 (dd, J = 13.4, 5.4 Hz, 1H), 3.66-3.53 (m, 8H), 3.24-3.15 |
| | (m, 1H), 2.84 (dd, J = 15.5, 3.9 Hz, 1H), 2.74-2.61 (m, 1H), 2.33 (s, 3H), 2.26 (s, 3H). |
| 342 | MS m/z 439.0 [M + H]⁺; ¹H NMR (400 MHz, DMSO-d₆) δ: 11.09 (s, 1H), 7.50 (d, |
| | J = 2.0 Hz, 1H), 7.38 (s, 1H), 7.29 (d, J = 8.4 Hz, 1H), 7.06 (dd, J = 8.6, 2.0 Hz, |
| | 1H), 6.99 (t, J = 7.4 Hz, 1H), 6.73 (t, J = 7.4 Hz, 1H), 6.48 (s, 1H), 4.93-4.90 (m, |
| | 1H), 3.31-3.22 (m, 1H), 2.79-2.76 (m, 2H), 2.26 (s, 3 H), 2.25 (d, J = 0.8 Hz, 6 H). |
| 347 | MS m/z 440.1 [M + H]⁺; ¹H NMR (DMSO-d₆) δ: 11.15 (s, 1H), 7.53 (d, J = 2.0 Hz, |
| | 1H), 7.33-7.30 (m, 2H), 7.08 (dd, J = 8.8, 2 Hz, 1H), 7.02 (t, J = 7.6 Hz, 1H), 6.76 (t, |
| | J = 6.8 Hz, 1H), 4.96 (dd, J = 13.2 Hz, 4.8 Hz, 1H), 3.32-3.24 (m, 1H), 2.90-2.78 (m, |
| | 2H), 2.34 (d, J = 1.6 Hz, 6H), 2.25 (d, J = 1.2 Hz, 3H) |

### Example 11

### Preparation of Compound 284

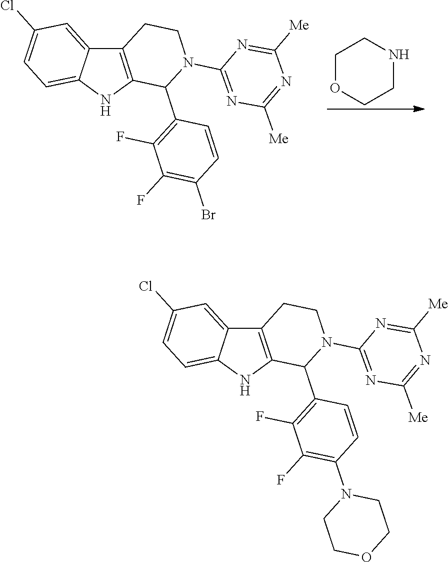 [SMILES: Cc1nc(C)nc(N2CCc3c([nH]c4ccc(Cl)cc34)C2c2ccc(Br)c(F)c2F)n1 ; C1COCCN1 ; Cc1nc(C)nc(N2CCc3c([nH]c4ccc(Cl)cc34)C2c2ccc(N3CCOCC3)c(F)c2F)n1]

To a screw-cap vial was added 1-(4-bromo-2,3-difluoro-phenyl)-6-chloro-2-(4,6-dimethyl-1,3,5-triazin-2-yl)-1,3,4,9-tetrahydropyrido[3,4-b]indole (100 mg, 0.20 mmol), Pd(dba), (18 mg, 0.020 mmol), RuPhos (10 mg, 0.020 mmol) and cesium carbonate (129 mg, 0.40 mmol). The vial was flushed with nitrogen. Added 1,4-dioxane (5 mL) and morpholine (23 mg, 0.26 mmol) via syringe. The reaction mixture was stirred at 100° C. for three hours. After cooling to RT, the mixture was concentrated in vacuo and purified by silica gel chromatography to afford Compound 284 (65 mg, 0.13 mmol, 64.21% yield) as a white solid. ESI-MS: m/z 511.1 [M+H]⁺; ¹H NMR (500 MHz, DMSO-d₆) δ 11.14 (s, 1H), 7.53 (d, J=2.1 Hz, 1H), 7.36-7.24 (m, 2H), 7.09 (ddh, J=8.6, 2.1 Hz, 1H), 6.86-6.60 (m, 2H), 4.95 (dd, J=13.4, 5.4 Hz, 1H), 3.73 (dd, J=6.1, 3.3 Hz, 4H), 3.30-3.17 (m, 1H), 3.17-2.80 (m, 4H), 2.90-2.64 (m, 2H), 2.35 (d, J=5.1 Hz, 6H).

### Example 12

### Synthesis of Compound 497

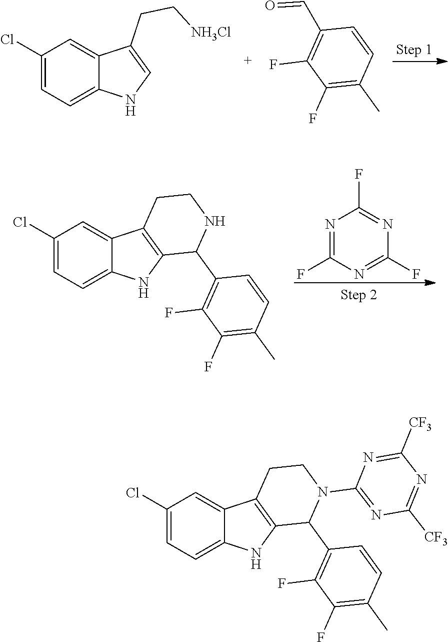

Step 1: A 100 mL single neck round-bottomed flask was charged with a solution of 2-(5-chloro-1H-indol-3-yl)ethanamine; hydrochloride (1.2 g, 5.2 mmol) in 1,2-dichloroethane (10.0 mL, 126 mmol) followed by 2,3-difluoro-4-methyl-benzaldehyde (1.0 g, 6.4 mmol) and trifluoroacetic acid (1.0 mL, 13 mmol). The mixture was allowed to stir at 90° C. for 3 h. After being allowed to cool to ambient temperature, the mixture was dried under reduced pressure to give a dark residue which was washed with ethyl acetate:petroleum ether equals 1:10 (2×60.0 mL). The combined organic washing solutions were evaporated to dryness affording 6-chloro-1-(2,3-difluoro-4-methyl-phenyl)-2,3,4,9-tetrahydro-1H-pyrido[3,4-b]indole (1.2 g, 3.6 mmol, 69% yield) This material was used without further purification.

Step 2: To a 100 mL three neck round-bottomed flask with a solution of 18-Crown-6 (80.0 mg, 0.297 mmol), 2,4,6-trifluoro-1,3,5-triazine (0.5 mL, 6 mmol) and (trifluoromethyl)trimethylsilane (2.0 mL, 13 mmol) in tetrahydrofuran (20.0 mL) were added Cesium fluoride (1.0 g, 6.6 mmol) in several portions over 30 minutes at 0° C. After that, the mixture was stirred for 30 minutes at 0° C. Then 6-chloro-1-(2,3-difluoro-4-methyl-phenyl)-2,3,4,9-tetrahydro-1H-pyrido[3,4-b]indole (A, 500 mg, 1.502 mmol) was added at 0° C. and the reaction mixture was stirred at 0° C. for 30 minutes. Then the mixture was filtered and the filtrate was concentrated to give a residue, which was purified by column flash (petroleum ether:ethyl acetate=10:1) to give the desired product Compound 497 (C, 100 mg, 0.1825 mmol, 12.15% yield) as a white solid material.

MS m/z 548.0 [M+H]⁺; ¹H NMR (400 MHz, DMSO-d₆) δ 11.20 (s, 1H), 7.59 (d, J=2.0 Hz, 1H), 7.34 (d, J=8.4 Hz, 1H), 7.23 (s, 1H), 7.05-7.12 (m, 2H), 6.84-6.88 (m, 1H), 4.93-4.97 (m, 1H), 3.54-3.59 (m, 1H), 2.91-3.06 (m, 2H), 2.27 (d, J=1.6 Hz, 3H).

Using the procedure described for Compound 497 above, additional compounds described herein were prepared by substituting the appropriate starting materials, suitable reagents and reaction conditions, obtaining compounds such as those selected from:

| Cpd | Data |
|---|---|
| 375 | MS: m/z 527.0 [M + H]⁺; ¹H NMR (400 MHz, DMSO) δ 11.14 (s, 1H), 8.77 (d, J = |
| | 4.0 Hz, 1H), 8.06 (dd, J = 5.8, 2.0 Hz, 1H), 7.53 (d, J = 2.0 Hz, 1H), 7.39 (t, J = |
| | 7.2 Hz, 1H), 7.30 (d, J = 8.4 Hz, 1H), 7.10-7.06 (m, 2H), 6.93 (br, 1H), 6.50 (d, J = |
| | 8.8 Hz, 1H), 4.45 (br, 1H), 4.79 (s, 1H), 4.63 (s, 1H), 3.76-3.72 (m, 1H), 3.58 (t, |
| | J = 7.4 Hz, 1H), 3.41-3.39 (m, 2H), 3.20-3.16 (m, 1H), 2.94-2.76 (m, 2H), 1.87- |
| | 1.80 (m, 2H). |
| 376 | MS: m/z 487.9 [M + H]⁺; ¹H NMR (400 MHz, DMSO) δ11.15(s, 1H), 8.05(d, |
| | J = 5.6 Hz, 1H), 7.53 (s, 1H), 7.29-7.37 (m, 2H), 7.07 (d, J = 6.8 Hz, 1H), 7.03 (s, 1H), |
| | 6.48 (d, J = 8.8 Hz, 1H),), 4.97 (q, J = 5.0 Hz, 1H) 4.78 (s, 1H), 4.62 (s, 1H), 3.74 (t, |
| | J = 6.4 Hz, 1H), 3.58 (t, J = 6.4 Hz, 1H), 3.39 (d, J = 10.0 Hz, 1H), 3.15-3.24 (m, 2H), |
| | 2.89 (d, J = 15.2 Hz, 1H), 2.71-2.79 (m, 1H), 2.39 (s, 3H), 2.32 (s, 3H), 1.83 (q, J = |
| | 11.8 Hz, 2H), |
| 377 | MS m/z 488.0 [M + H]⁺; ¹H NMR (400 MHz, DMSO) δ 11.15 (s, 1H), 8.05 (dd, J = |
| | 7.0, 2.4 Hz, 1H), 7.52 (d, J = 2.0 Hz, 1H), 7.38-7.34 (m, 1H), 7.30 (d, J = 8.4 Hz, |
| | 1H), 7.07 (dd, J = 8.8, 2.0 Hz, 1H), 7.02 (d, J = 2.8 Hz, 1H), 7.48 (d, J = 8.8 Hz, |
| | 1H), 4.98 (dd, J = 13.2, 5.2 Hz, 1H), 4.79 (s, 1H), 4.63 (s, 1H), 3.76-3.72 (m, 1H), |
| | 3.56 (t, J = 6.4 Hz, 1H), 3.40 (d, J = 10.0 Hz, 1H), 3.24-3.16 (m, 2H), 2.91-2.71 (m, |
| | 2H), 2.40 (d, J = 0.4 Hz, 3H), 2.32 (s, 3H), 1,87-1.81 (m, 2H). |
| 378 | MS: m/z 400.0 [M + H]⁺; ¹H NMR (400 MHz, DMSO) δ 11.13 (s, 1H), 8.74 (d, |
| | J = 2.8 Hz, 1H), 7.52 (d, J = 2.0 Hz, 1H), 7.32 (d, J = 8.4 Hz, 1H), 7.22 (d, J = 4.8 Hz, |
| | 1H), 7.15-7.20 (m, 4H), 6.97-7.09 (m, 2H), 4.81 (s, 1H), 3.20-3.27 (m, 1H), 2.78- |
| | 2.88 (m, 2H), 2.27 (s, 3H). |
| 380 | MS: m/z 490.0 [M + H]⁺; ¹H NMR (400 MHz, DMSO) δ 11.17(s, 1H), 8.30(s, 2H), |
| | 7.54(d, J = 1.2 Hz, 1H), 7.32 (d, J = 8.4 Hz, 1H), 7.08 (q, J = 6.8 Hz, 1H), 6.91 (s, |
| | 1H), 5.02 (d, J = 8.4 Hz, 1H), 3.69(s, 4H), 3.27(m, 1H), 2.91(q, J = 12.4 Hz, 1H), |
| | 2.75 (m, 1H), 2.40(s, 3H), 2.31(q, J = 4.8 Hz, 7H), 2.18(s, 3H). |
| 386 | MS m/z 510.0 [M − H]⁻; ¹H NMR (DMSO-d₆) δ: 11.25 (s, 1H), 7.56 (s, 1H), 7.33 (d, |
| | J = 6.3 Hz, 1H), 7.21 (br s, 4H), 7.16-6.95 (m, 2H), 5.08-4.74 (m, 1H), 3.40 (t, J = |
| | 13.1 Hz, 1H), 3.10-2.77 (m, 2H), 2.28 (s, 3H). |
| 388 | MS m/z 584.8 [M + H]⁺; ¹H NMR (DMSO-d₆) δ: 11.11 (s, 1H), 8.33 (s, 2H), 7.59 |
| | (br s, 1H), 7.35-7.32 (m, 1H), 7.12-7.09 (m, 1H), 6.85 (t, J = 3.6 Hz, 1H), 4.96 |
| | (dd, J = 13.5, 6.5 Hz, 1H), 3.95-3.49 (m, 9H), 3.03 (d, J = 15.4 Hz, 1H), 2.86 (q, |
| | J = 13.3, 10.3 Hz, 1H). |
| 404 | MS m/z 584.3 [M + H]⁺; ¹H NMR (DMSO-d₆) δ: 11.00 (s, 1H), 7.96 (d, J = 2.5 Hz, |
| | 1H), 7.40 (d, J = 2.1 Hz, 1H), 7.27 (dd, J = 8.9, 2.5 Hz, 1H), 7.16 (d, J = 8.6 Hz, |
| | 1H), 6.93 (dd, J = 8.6, 2.1 Hz, 1H), 6.78 (s, 1H), 6.65 (d, J = 8.9 Hz, 1H), 4.75 (dd, |
| | J = 13.4, 5.3 Hz, 1H), 3.48 (t, J = 4.8 Hz, 4H), 3.39-3.20 (m, 5H), 2.85 (dd, J = |
| | 15.3, 4.0 Hz, 1H), 2.69 (ddd, J = 16.0, 11.5, 5.4 Hz, 1H). |

### Example 13

### Preparation of Compound 358

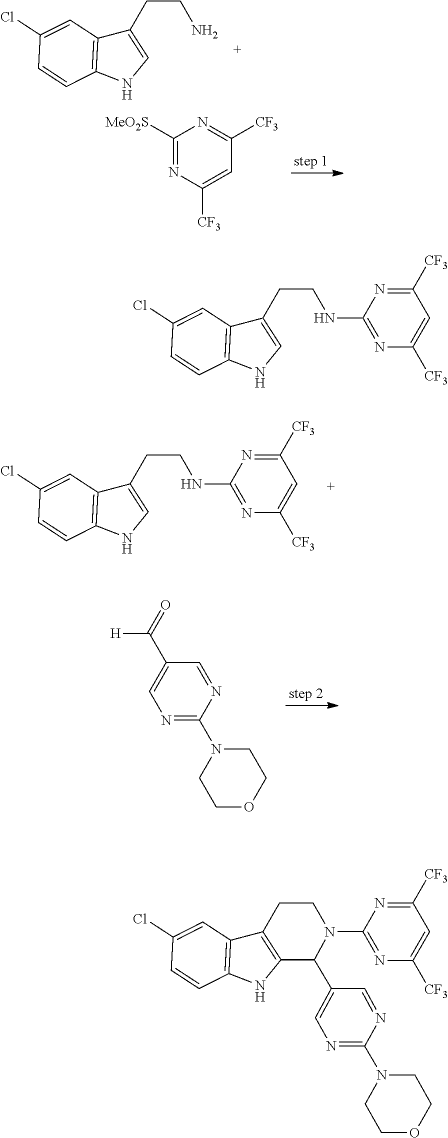

Step 1: A 100 mL round bottom flask equipped with a stir bar was charged with 2-(5-chloro-1H-indol-3-yl)ethanamine (580 mg, 2.980 mmol), tetrahydrofuran (10 mL), N,N-diisopropylamine (1.0 mL, 5.960 mmol), followed by 2-methylsulfonyl-4,6-bis-(trifluoromethyl)pyrimidine (964 mg, 3.280 mmol). The flask was capped with a nitrogen inlet and allowed to stir for 16 h at 22° C., after which volatiles were removed under reduced pressure. The resulting dark residue was subjected to silica-gel chromatography (petroleum ether:ethyl acetate, gradient 0 to 40%) to afford the desired product N-[2-(5-chloro-1H-indol-3-yl)ethyl]-4,6-bis(trifluoromethyl)pyrimidin-2-amine (350 mg, 0.856 mmol, 28.7% yield) as an amorphous yellow solid.

Step 2: A 8 mL screw-cap vial was subsequently charged with N-[2-(5-chloro-1H-indol-3-yl)ethyl]-4,6-bis(trifluoromethyl)pyrimidin-2-amine (80 mg, 0.196 mmol), 1,2-dichloroethane (1 mL), 2-morpholinopyrimidine-5-carbaldehyde (50 mg, 0.259 mmol) and trifluoroacetic acid (0.1 mL, 1 mmol), tighty capped and allowed to stir for 6 h at 95° C. After being allowed to cool to ambient temperature the reaction mixture was quenched with NaHCO₃ aq (saturated, 10 mL) and extracted with EtOAc (3×20 mL). The combined organic phases were dried over MgSO₄, filtered, volatiles removed affording a dark oil. After purification by prep-HPLC (water:acetonitrile gradient 10-100%) Compound 358 (85 mg, 0.146 mmol, 74% yield) was isolated as a white amorphous solid. MS m/z 582.0 [M−H]⁻; ¹H NMR (400 MHz, DMSO-d₆) δ: 11.09 (s, 1H), 8.34 (s, 2H), 7.57-7.55 (m, 2H), 7.32 (d, J=8.8 Hz, 1H), 7.11-7.09 (m, 1H), 6.76 (s, 1H), 4.99-4.94 (m, 1H), 3.67-3.61 (m, 8H), 3.54-3.51 (m, 1H), 3.01-2.95 (m, 2H).

Using the procedure described for Compound 358 above, additional compounds described herein were prepared by substituting the appropriate starting materials, suitable reagents and reaction conditions, obtaining compounds such as those selected from:

| Cpd | Data |
|---|---|
| 200 | MS m/z 534 [M + H]⁺; ¹H NMR (400 MHz, DMSO-d₆) δ: 11.39 (s, 1H), 8.80 (d, |
| | J = 4.0 Hz, 1H), 8.39 (s, 2H), 7.19-7.12 (m, 3H), 6.83 (s, 1H), 5.02-5.01 (m, 1H), |
| | 3.68-3.63 (m, 8H), 3.47-3.44 (m, 1H), 3.10-2.94 (m, 2H). |
| 202 | MS m/z 448 [M + H]⁺; ¹H NMR (DMSO-d₆, 400 MHz) δ: 11.49 (s, 1H), 8.81 (d, |
| | J = 4.4 Hz, 1H), 8.65 (d, J = 1.6 Hz, 1H), 8.54 (dd, J1 = 3.2 Hz, J2 = 1.2 Hz, 1H), 7.70 |
| | (d, J = 8 Hz, 1H), 7.41-7.38 (m, 1H), 7.19-7.13 (m, 3H), 7.07 (br, 1H), 4.96- |
| | 4.95(m,, 1H), 3.38-3.2(m, 1H), 3.09-3.00 (m, 2H). |
| 209 | MS m/z 563.0 [M + H]⁺; ¹H NMR (DMSO, 400 MHz) δ: 11.45(s, 1H), 7.57(s, 1H), |
| | 7.18(s, 1H), 7.16-7.12(m, 2H), 7.04(t, J = 7.6 Hz, 1H), 6.87(t, J = 6.8 Hz, 1H), 4.92- |
| | 4.87(m, 1H), 3.59-3.51(m, 1H), 3.13-3.04(m, 2H), 2.26(s, 3H). |
| 251 | MS m/z 515.8 [M + H]⁺; ¹H NMR (400 MHz, DMSO) δ 11.09(s, 1H), 8.79(d, J = |
| | 4.8 Hz, 1H), 8.34(s, 2H), 7.55 (d, J = 2.4 Hz, 1H), 7.31 (d, J = 8.8 Hz, 1H), 7.11 |
| | (d, J = 4.8 Hz, 1H), 7.09 (dd, J = 8.0, 2.4 Hz, 1H), 6.81 (s, 1H), 5.01 (s, 1H), |
| | 3.67-3.65 (m, 4 H), 3.62-3.60(m, 4H), 3.44-3.37(m, 1H), 2.97-2.92 (m, 1H), 2.84- |
| | 2.76(m, 1H). |
| 357 | MS m/z 581.0 [M − H]⁻; ¹H NMR (400 MHz, DMSO-d₆) δ: 11.14 (s, 1H), 8.14 (d, |
| | J = 2.4 Hz, 1H), 7.56 (d, J = 2.0 Hz, 1H), 7.54 (s, 1H), 7.45 (dd, J = 9.0, 2.0 Hz, |
| | 1H), 7.31 (d, J = 8.8 Hz, 1H), 7.09 (dd, J = 9.4, 2.0 Hz, 1H), 6.88 (s, 1H), 6.82 (d, |
| | J = 9.2 Hz, 1H), 4.95-4.91 (m, 1H), 3.67-3.64 (m, 4H), 3.47-3.44 (m, 1H), 3.42- |
| | 3.39 (m, 4H), 3.00-2.98 (m, 2H). |
| 360 | MS m/z 509.0 [M − H]⁻; ¹H NMR (400 MHz, CDCl3) δ: 7.75 (s, 1H), 7.53-7.53 (d, |
| | J = 2.0 Hz, 1H), 7.33-7.31 (d, J = 8.0 Hz, 2H), 7.18-7.12 (m, 4H), 7.05 (s, 1H), 7.04 |
| | (s, 1H), 5.13-5.09 (dd, J = 4.0, 13.2 Hz, 1H), 3.51-3.43 (m, 1H), 2.98-2.89 (m, 2H), |
| | 2.32 (s, 3H). |
| 365 | MS m/z 479.0 [M + H]⁺; ¹H NMR (DMSO-d₆) δ: 11.13 (s, 1H), 8.38 (s, 1H), 8.02 |
| | (s, 1H), 7.57 (br d, J = 8.5 Hz, 1H), 7.53 (s, 1H), 7.32 (d, J = 8.5 Hz, 1H), 7.08 (br d, |
| | J = 8.5 Hz, 1H), 6.95 (br d, J = 8.8 Hz, 1H), 6.91 (s, 1H), 4.68-5.07 (m, 1H), 3.69 (br |
| | t, J = 4.4 Hz, 4H), 3.45 (br t, J = 4.4 Hz, 4H), 3.17-3.32 (m, 1H), 2.71-2.94 (m, 2H), |
| | 2.38 (s, 3H). |
| 366 | MS m/z 479.9 [M + H]⁺; ¹H NMR (DMSO-d₆) δ: 11.02-11.16 (m, 1H), 8.38 (s, 1H), |
| | 8.32 (s, 2H), 7.53 (s, 1H), 7.31 (d, J = 8.5 Hz, 1H), 7.03-7.13 (m, 1H), 6.74-6.84 (m, |
| | 1H), 4.79-5.07 (m, 2H), 3.64 (br dd, J = 12.5, 4.0 Hz, 8H), 3.21-3.44 (m, 1H), 2.85 |
| | (br s, 2H), 2.38 (s, 3H). |
| 367 | MS m/z 493.0 [M + H]⁺; ¹H NMR (DMSO-d₆) δ: 11.10 (s, 1H), 8.03 (s, 1H), 7.42- |
| | 7.65 (m, 2H), 7.21-7.41 (m, J = 8.5 Hz, 1H), 7.08 (br d, J = 8.5 Hz, 1H), 6.81-7.01 |
| | (m, 2H), 4.88 (br dd, J = 13.5, 4.5 Hz, 1H), 3.69 (br t, J = 4.3 Hz, 4H), 3.37-3.54 (m, |
| | 4H), 3.09-3.37 (m, 1H), 2.66-2.94 (m, 2H), 2.34 (s, 6H). |
| 368 | MS m/z 494.0 [M + H]⁺; ¹H NMR (DMSO-d₆) δ: 11.06 (s, 1H), 8.32 (s, 2H), 7.53 |
| | (s, 1H), 7.23-7.36 (m, J = 8.5 Hz, 1H), 7.06 (s, 1H), 6.79 (s, 1H), 4.93 (br dd, |
| | J = 13.5, 5.0 Hz, 1H), 3.64 (br dd, J = 12.0, 4.0 Hz, 8H), 3.16-3.36 (m, 1H), 2.66- |
| | 2.96 (m, 2H), 2.34 (d, J = 1.8 Hz, 6H). |
| 381 | MS m/z 597.0 [M + H]⁺; ¹H NMR (400 MHz, CDCl₃) δ: 8.37 (s, 2H), 7.83 (s, 1H), |
| | 7.55 (d, J = 1.6 Hz, 1H), 7.22 (d, J = 8.4 Hz, 1H), 7.17 (q, J = 6.8 Hz, 1H), 7.11 |
| | (s, 1H), 6.94 (s, 1H), 5.19 (q, J = 10.8 Hz, 1H), 3.87 (s, 4H), 3.42 (m, 1H), 2.97 |
| | (m, 2H), 2.50 (s, 4H), 2.38 (s, 3H). |

### Example 14

### Preparation of Compound 144

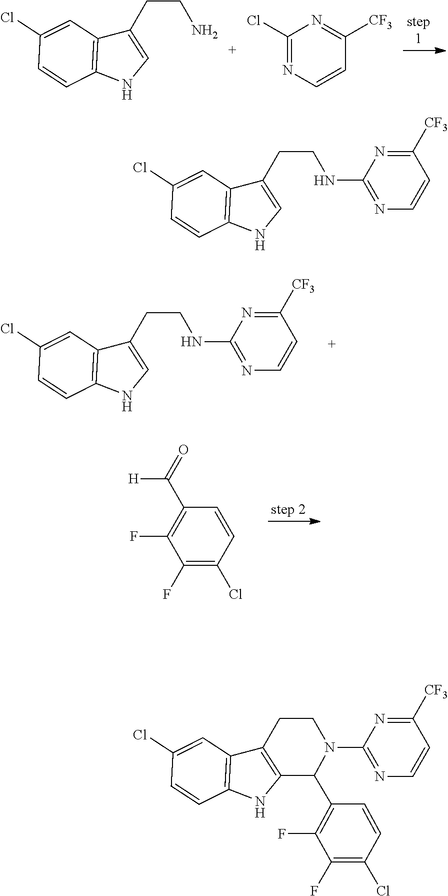 [SMILES: NCCc1c[nH]c2ccc(Cl)cc12 ; FC(F)(F)c1ccnc(Cl)n1 ; FC(F)(F)c1ccnc(NCCc2c[nH]c3ccc(Cl)cc23)n1 ; FC(F)(F)c1ccnc(NCCc2c[nH]c3ccc(Cl)cc23)n1 ; O=Cc1ccc(Cl)c(F)c1F ; Fc1c(Cl)ccc(C2c3[nH]c4ccc(Cl)cc4c3CCN2c2nccc(C(F)(F)F)n2)c1F]

Step 1: A 100 mL round bottomed flask equipped with a reflux condensor was subsequently charged with 2-(5-chloro-1H-indol-3-yl)ethanamine (5.0 g, 26 mmol), 1-butanol (25 mL), triethylamine (7.2 mL, 51 mmol) and 2-chloro-4-(trifluoromethyl)pyrimidine (7.0 g, 38 mmol). The reaction mixture was allowed to stir at reflux for 16 h, after which volatiles were evaporated under reduced pressure and the resulting dark oily residue subjected to silica-gel chromatography (petroleum ether:EtOAc gradient 0 to 40%). After purification N-[2-(5-chloro-1H-indol-3-yl)ethyl]-4-(trifluoromethyl)pyrimidin-2-amine (7.0 g, 21 mmol, 80% yield) was isolated as an off-white amorphous solid.

Step 2: An 8 mL vial equipped with a stir bar was charged with N-[2-(5-chloro-1H-indol-3-yl)ethyl]-4-(trifluoromethyl)pyrimidin-2-amine (50 mg, 0.147 mmol) and 2-butanol (3 mL), then 4-chloro-2,3-difluoro-benzaldehyde (26 mg, 0.147 mmol) was added by micro syringe followed by toxic acid (16 mg, 0.093 mmol). The vial was allowed to stir at 95° C. for 1 h. After being allowed to cool to ambient temperature, volatiles were removed under reduced pressure and the resulting dark residue subjected to purification by preparatory scale HPLC. After purification Compound 144 (57 mg, 0.778 mmol, 77.8% yield) was isolated as an amorphous white solid. MS m/z 496.9 [M−H]⁻; ¹H NMR (400 MHz, DMSO-d₆) δ: 11.14 (s, 1H), 8.79 (d, J=4.8 Hz, 1H), 7.56 (d, J=2.0 Hz, 1H), 7.38-7.31 (m, 2H), 7.28-7.20 (m, 2H), 7.15 (d, J=5.2 Hz, 1H), 7.09 (dd, J=8.8, 2.0 Hz, 1H), 6.92 (t, J=11.0 Hz, 1H), 4.97-4.84 (m, 1H), 3.48-3.36 (2, 1H), 2.97-2.78 (m, 2H).

Using the procedure described for Compound 144 above, additional compounds described herein were prepared by substituting the appropriate starting materials, suitable reagents and reaction conditions:

| Cpd | Data |
|---|---|
| 1 | MS: m/z 413.0 [M + H]⁺; ¹H NMR (DMSO, 400 MHz), δ: 11.26(s, 1H), 8.52(s, 2H), |
| | 7.52(d, J = 2.0 Hz, 1H), 7.35-7.31 (m, 3 H), 7.18 (t, J = 8.8 Hz, 2H), 7.08 (dd, J = |
| | 8.4, 2.0 Hz, 1H), 7.07 (s, 1H), 4.84-4.80 (m, 1H), 3.22-3.14 (m, 1H), 2.83-2.79 (m, 2H). |
| 2 | MS: m/z 393.1 [M + H]⁺; ¹H NMR (400 MHz, DMSO) δ 11.22 (s, 1H), 8.31 (s, |
| | 2H), 7.50 (d, J = 2.0 Hz, 1H), 7.33-7.30(m, 3H), 7.19-7.06(m, 4H), 4.83 (d, J = |
| | 12.0 Hz, 1H), 3.16-3.09 (m, 1H), 2.78 (d, J = 4.0 Hz, 2H), 2.10 (s, 3H). |
| 3 | MS: m/z 397.0 [M + H]⁺; ¹H NMR (400 MHz, DMSO-d₆) δ 11.23(s, 1H), 8.54(s, |
| | 2H), 7.52-7.51 (m, 1H), 7.35-7.31 (m, 3H), 7.20-7.16 (m, 2H), 7.09-7.04 (m, 2H), |
| | 4.80-4.77(m, 1H), 3.22-3.14(m, 1H), 2.81-2.79 (m, 2H). |
| 4 | MS: m/z 413.0 [M + H]⁺; ¹H NMR (400 MHz, DMSO-d₆) δ 11.34(s, 1H), 8.52(s, |
| | 2H), 7.67-7.65 (m, 1H), 7.35-7.31 (m, 3H), 7.20-7.16 (m, 2H), 7.05 (s, 1H), 4.83- |
| | 4.79 (m, 1H), 3.20-3.13 (m, 1H), 2.85-2.75 (m, 2H). |
| 5 | MS: m/z 415.0 [M + H]⁺; ¹H NMR (400 MHz, DMSO-d₆) δ 11.32(s, 1H), 8.54(s, |
| | 2H), 7.66-7.64 (m, 1H), 7.34-7.31 (m, 3H), 7.20-7.16 (m, 2H), 7.03 (s, 1H), 4.80- |
| | 4.77 (m, 1H¹H), 3.20-3.13 (m, 1H), 2.82-2.80 (m, 2H). |
| 6 | MS: m/z 411.0 [M + H]⁺; ¹HNMR (400 MHz, DMSO) δ 11.29 (s, 1H), 8.52 (s, 2H), |
| | 7.63 (d, J = 8.0 Hz, 1H), 7.31 (d, J = 12.0 Hz, 1H), 7.19-7.13 (m, 4H), 7.00 (s, 1H), |
| | 4.79-4.75 (m, 1H), 3.22-3.15 (m, 1H), 2.80-2.79 (m, 2H), 2.27 (s, 3H) |
| 7 | ¹H NMR (DMSO-d₆) δ: 11.13-11.28 (m, 1H), 7.52-7.57 (m, 1H), 7.29-7.36 (m, |
| | 1H), 7.18 (s, 4H), 7.06-7.11 (m, 1H), 7.03 (s, 1H), 6.93 (s, 1H), 4.69-4.78 (m, 1H), |
| | 3.19-3.30 (m, 1H), 2.76-2.94 (m, 2H), 2.24-2.32 (m, 3H) |
| 8 | MS: m/z 428.0 [M + H]⁺; ¹H NMR (400 MHz, DMSO) δ 11.26 (s, 1H), 8.55 (s, |
| | 2H), 8.10 (d, J = 4.0 Hz, 1H), 7.66 (d, J = 8.0 Hz, 1H), 7.59 (dd, J = 8.0, 4.0 Hz, |
| | 1H), 7.32 (d, J = 12.0 Hz, 1H), 6.94 (s, 1H), 6.79 (d, J = 8.0 Hz, 1H), 4.84 (dd, J = |
| | 16.0, 8.0 Hz, 1H), 3.81 (s, 3H), 3.29-3.15 (m, 1H), 2.82-2.78 (m, 2H). |
| 9 | MS: m/z 411.0 [M + H]⁺; ¹H NMR (400 MHz, DMSO-d₆) δ 11.31(s, 1H), 8.30- |
| | 8.29(m, 1H), 7.65-7.63 (m, 1H), 7.34-7.29(m, 3H), 7.19-7.15 (m, 3H), 6.59-6.58 |
| | (m, 1H), 4.92-4.88 (m, 1H), 3.14-3.08 (m, 1H), 2.80-2.76 (m, 2H), 2.34 (s, 3H). |
| 10 | MS m/z 409.4 [M + H]⁺. ¹H NMR (acetone) δ: 10.22 (br s, 1H), 8.36 (d, J = 4.9 Hz, |
| | 1H), 7.55 (d, J = 1.8 Hz, 1H), 7.38 (d, J = 8.5 Hz, 1H), 7.28 (d, J = 8.2 Hz, 2H), 7.15 |
| | (d, J = 7.9 Hz, 3H), 7.11 (dd, J = 8.5, 2.1 Hz, 1H), 6.70 (d, J = 5.2 Hz, 1H), 4.93-5.03 |
| | (m, 1H), 3.36 (ddd, J = 13.4, 10.8, 5.3 Hz, 1H), 2.88-2.99 (m, 2H), 2.30 (s, 3H), |
| | 2.07 (dt, J = 4.3, 2.3 Hz, 1H), 1.25-1.36 (m, 1H), 1.13 (t, J = 7.0 Hz, 1H), 0.86-0.99 |
| | (m, 1H) |
| 119 | MS: m/z 411.0 [M + H]⁺; ¹H NMR (400 MHz, DMSO-d₆) δ 11.31 (s, 1H), 8.55(s, |
| | 2H), 7.66 (d, J = 7.2 Hz, 1H), 7.42 (d, J = 8.4 Hz, 2H), 7.35(d, J = 10.4 Hz, 1H), |
| | 7.31(d, J = 8.8 Hz, 2H), 7.02 (s, 1H), 4.79 (d, J = 13.2 Hz, 1H), 3.20-3.12 (m, 1H), |
| | 2.81-2.80 (m, 2H) |
| 12 | MS: m/z 433.0 [M + H]⁺; ¹H NMR (DMSO, 400 MHz), δ 11.24 (s, 1H), 8.55 (s, |
| | 2H), 7.65 (d, J = 7.6 Hz, 1H), 7.35-7.33(m, 1H), 7.31-7.29(m, 2H), 7.05-6.94 (m, 2H), |
| | 4.72 (d, J = 13.2 Hz, 1H), 3.26-3.18 (m, 1H), 2.80-2.78 (m, 2H). |
| 13 | MS: m/z 413.0 [M + H]⁺; ¹H NMR (400 MHz, DMSO-d₆) δ 11.24(s, 1H), 8.43- |
| | 8.42(m, 1H), 7.53 (s, 1H), 7.34-7.32 (m, 3H), 7.22-7.17(m, 2H), 7.10-7.08 (m, |
| | 2H), 6.84-6.82 (m, 1H), 4.81 (s, 1H), 3.21-3.16 (m, 1H), 2.87-2.78 (m, 2H). |
| 14 | MS: m/z 442.0 [M + H]⁺; ¹H NMR (400 MHz, DMSO-d₆) δ 11.20(s, 1H), 8.54(s, |
| | 2H), 8.26 (s, 2H), 7.67-7.65 (m, 1H), 7.33-7.31(m, 1H), 6.75 (s, 1H), 4.90-4.86 |
| | (m. 1H), 3.32-3.25(m, 1H), 3.07(s, 6H), 2.89-2.76(m, 2H). |
| 15 | MS: m/z 431.0 [M + H]⁺; ¹H NMR (400 MHz, DMSO-d₆) δ 11.32(s, 1H), 8.43- |
| | 8.42(m, 1H), 7.68-7.66 (m, 1H), 7.36-7.32(m, 3H), 7.22-7.17 (m, 2H), 7.04 (s, |
| | 1H), 6.84-6.82 (m, 1H), 4.81 (s, 1H), 3.21-3.15 (m, 1H), 2.87-2.74 (m, 2H). |
| 16 | MS m/z 461.5 [M − H]⁻; ¹H NMR (DMSO-d₆) δ: 11.29 (s, 1H), 7.67 (d, J = 7.3 Hz, |
| | 1H), 7.34 (d, J = 10.1 Hz, 1H), 7.18 (s, 4H), 7.03 (s, 1H), 6.92 (s, 1H), 4.74 (br dd, |
| | J = 13.6, 5.0 Hz, 1H), 3.18-3.29 (m, 1H), 3.04-3.16 (m, 1H), 2.75-2.91 (m, 2H), |
| | 2.28 (s, 3H) |
| 18 | MS: m/z 436.0 [M + H]⁺; ¹H NMR (400 MHz, DMSO-d₆) δ 11.34 (s, 1H), 8.18 (d, |
| | J = 6.0 Hz, 1H), 7.88-7.86 (m, 2H), 7.71 (d, J = 7.2 Hz, 1H), 7.50-7.48 (m, 2H), 7.38 |
| | (d, J = 10.0 Hz, 1H), 7.13-7.12 (m, 2H), 4.55-4.24 (m, 1H), 3.31-3.25 (m, 1H), |
| | 2.87-2.84 (m, 2H). |
| 19 | MS: m/z 425.0 [M + H]⁺; ¹H NMR (400 MHz, DMSO-d₆) δ 11.29 (s, 1H), 7.64 (d, |
| | J = 7.6 Hz, 1H), 7.34-7.30 (m, 3H), 7.18-7.13 (m, 3H), 6.47 (s, 1H), 4.94-4.91 (m, |
| | 1H), 3.11-3.04 (m, 1H), 2.80-2.75 (m, 2H), 2.29 (s, 6H). |
| 20 | MS: m/z 411.0 [M + H]⁺; ¹H NMR (400 MHz, DMSO-d₆) δ 11.50 (s, 1H), 8.53 (s, |
| | 2H), 7.20-7.10 (m, 6H), 7.02 (s, 1H), 4.78-4.74 (m, 1H), 3.25-3.18 (m, 1H), 3.02- |
| | 2.91 (m, 2H), 2.27 (s, 3H). |
| 21 | MS: m/z 427.0 [M + H]⁺; ¹H NMR (400 MHz, DMSO-d₆) δ 11.52 (s, 1H), 8.52 (s, |
| | 2H), 7.20-7.10 (m, 6H), 7.04 (s, 1H), 4.82-4.78 (m, 1H), 3.25-3.18 (m, 1H), 2.98- |
| | 2.95 (m, 2H), 2.27 (s, 3H). |
| 26 | MS: m/z 431.0 [M + H]⁺; ¹H NMR (400 MHz, DMSO-d₆) δ 11.52 (s, 1H), 8.55 (s, |
| | 2H), 7.43-7.41 (m, 2H), 7.33-7.31(m, 2H), 7.19-7.11 (m, 2H), 7.03 (s, 1H), 4.80- |
| | 4.76 (m, 1H), 3.23-3.16 (m, 1H), 2.99-2.95 (m, 2H). |
| 27 | MS: m/z 447.0 [M + H]⁺; ¹H NMR (400 MHz, DMSO-d₆) δ 11.53 (s, 1H), 8.53 (s, |
| | 2H), 7.44-7.41 (m, 2H), 7.33-7.31(m, 2H), 7.19-7.12 (m, 2H), 7.05 (s, 1H), 4.84- |
| | 4.80 (m, 1H), 3.23-3.16 (m, 1H), 2.98-2.96 (m, 2H). |
| 28 | MS: m/z 427.0 [M + H]⁺; ¹H NMR (400 MHz, DMSO-d₆), δ 11.50 (s, 1H), 8.41 (d, |
| | J = 4.8 Hz, 1H), 7.21-7.11 (m, 6H), 7.02 (b, 1H), 6.81 (d, J = 5.2 Hz, 1H), 4.81 (m, 1H), |
| | 3.27-3.20 (m, 1H), 2.99-2.97 (m, 2H), 2.28(s, 3H). |
| 29 | MS: m/z 446.9 [M + H]⁺; ¹H NMR (400 MHz, DMSO-d₆) δ 11.33 (s, 1H), 8.52 (s, |
| | 2H), 7.67-7.65 (m, 1H), 7.43-7.41 (m, 2H), 7.36-7.29 (m, 3H), 7.04(s, 1H), 4.84- |
| | 7.80 (m, 1H), 3.20-3.12 (m, 1H), 2.85-2.76 (m, 2H). |
| 30 | MS: m/z 431.0 [M + H]⁺; ¹H NMR (400 MHz, DMSO) δ 11.37 (s, 1H), 8.55 (s, |
| | 2H), 7.66 (d, J = 8.0 Hz, 1H), 7.40-7.27 (m, 5H), 7.01 (s, 1H), 4.80 (d, J = 12.0 |
| | Hz, 1H), 3.23-3.16 (m, 1H), 2.82-2.80 (m, 2H). |
| 31 | MS: m/z 437.0 [M + H]⁺; ¹H NMR (400 MHz, DMSO) δ (ppm), 11.21 (s, 1H), 8.45 |
| | (s, 2H), 7.56 (d, J = 6.8 Hz, 1H), 7.23 (d, J = 10.0 Hz, 1H), 7.10-7.08 (m, 2H), 6.97- |
| | 6.93 (m, 3H), 4.70 (d, J = 12.8 Hz, 1H), 3.18-3.09 (m, 1H), 2.72-2.71 (m, 2H), |
| | 1.86-1.72 (m, 1H), 0.92-0.74 (m, 2H), 0.55 (s, 2H). |
| 32 | MS: m/z 422.0 [M + H]⁺; ¹H NMR (400 MHz, DMSO) δ (ppm), 11.33 (s, 1H), 8.55 |
| | (s, 2H), 7.83 (d, J = 8.4 Hz, 2H), 7.66 (d, J = 7.6 Hz, 1H), 7.49 (d, J = 8.0 Hz, 2H), |
| | 7.36 (d, J = 10.0 Hz, 1H), 7.06 (s, 1H), 4.82-4.78 (m, 1H), 3.21-3.14 (m, 1H), 2.85- |
| | 2.76 (m, 2H). |
| 33 | MS: m/z 422.0 [M + H]⁺; ¹H NMR (400 MHz, DMSO) δ (ppm), 11.79 (s, 1H), 8.56 |
| | (s, 2H), 7.83 (d, J = 8.4 Hz, 2H), 7.48 (d, J = 8.0 Hz, 2H), 7.42 (s, 1H), 7.10-7.08 |
| | (m, 2H), 4.79-4.76(m, 1H), 3.18-3.11(m, 1H), 2.88-2.76 (m, 2H). |
| 34 | MS: m/z 441.0 [M + H]⁺; ¹H NMR (400 MHz, DMSO) δ 11.22 (s, 1H), 8.53 (s, |
| | 2H), 8.03 (d, J = 2.0 Hz, 1H), 7.64 (d, J = 8.0 Hz, 1H), 7.36 (dd, J = 8.0, 4.0 Hz, 1H), |
| | 7.29 (d, J = 12.0 Hz, 1H), 6.87 (s, 1H), 6.58 (d, J = 8.0 Hz, 1H), 4.82 (dd, J = 16.0, |
| | 4.0 Hz, 1H), 3.32-3.20(m, 1H), 2.97(s, 6H), 2.86-2.73(m, 2H). |
| 35 | MS: m/z 465.0 [M + H]⁺; ¹H NMR (400 MHz, DMSO) δ 11.34 (s, 1H), 8.56 (s, |
| | 2H), 7.73 (d, J = 8.0 Hz, 2H), 7.67 (d, J = 8.0 Hz, 1H), 7.53 (d, J = 8.1 Hz, 2H), 7.36 |
| | (d, J = 8.0 Hz, 1H), 7.09 (s, 1H), 4.81(d, J = 16.0 Hz, 1H), 3.22-3.15(m, 1H), 2.86- |
| | 2.78(m, 2H). |
| 36 | MS: m/z 433.0 [M + H]⁺; ¹H NMR (400 MHz, DMSO) δ 11.26 (s, 1H), 8.55 (s, |
| | 2H), 7.65 (d, J = 8.0 Hz, 1H), 7.43-7.38 (m, 1H), 7.35-7.32 (m, 2H), 7.15 (dd, J = |
| | 12.0, 8.0 Hz, 1H), 6.81 (t, J = 8.0 Hz, 1H), 4.77 (d, J = 12.0 Hz, 1H), 3.30-3.25 (m, |
| | 1H), 2.82-2.80 (m, 2H) |
| 37 | MS: m/z 433.0 [M + H]⁺; ¹H NMR (400 MHz, DMSO) δ 11.32 (s, 1H), 8.55 (s, |
| | 2H), 7.66 (d, J = 7.4 Hz, 1H), 7.43-7.32(m, 3H), 7.15-7.13(m, 1H), 6.98 (s, 1H), |
| | 4.82 (dd, J = 12.0, 4.0 Hz, 1H), 3.25-3.20(m, 1H), 2.82-2.79(m, 2H). |
| 38 | MS: m/z 427.0 [M + H]⁺; ¹H NMR (400 MHz, DMSO-d₆) δ 11.31 (s, 1H), 8.29 (d, |
| | J = 5.2 Hz, 1H), 7.64 (d, J = 7.2 Hz, 1H), 7.42-7.40 (m, 2H), 7.34-7.30 (m, 3H), 7.14 |
| | (s, 1H), 6.29 (d, J = 4.8 Hz, 1H), 4.92-4.89 (m, 1H), 3.13-3.06 (m, 1H), 2.80-2.76 |
| | (m, 2H), 2.34(s, 3H). |
| 39 | MS: m/z 446.9 [M + H]⁺; ¹H NMR (400 MHz, DMSO-d₆) δ 11.31 (s, 1H), 8.42 (d, |
| | J = 5.2 Hz, 1H), 7.47 (d, J = 7.2 Hz, 1H), 7.44-7.42 (m, 2H), 7.37-7.30 (m, 3H), 7.09- |
| | 6.96 (m, 1H), 6.83 (d, J = 5.2 Hz, 1H), 4.88-4.76 (m, 1H), 3.21-3.14 (m, 1H), 2.84- |
| | 2.80 (m, 2H). |
| 40 | MS: m/z 438.0 [M + H]⁺; ¹H NMR (400 MHz, DMSO-d₆) δ 11.31 (s, 1H), 8.75 |
| | (d, J = 4.8 Hz, 1H), 7.66 (d, J = 7.6 Hz, 1H), 7.45-7.43 (m, 2H), 7.37-7.30 (m, 3H), |
| | 7.26 (d, J = 4.8 Hz, 1H), 7.08-6.99 (m, 1H), 4.91-4.76 (m, 1H), 3.24-3.17 (m, 1H), |
| | 2.90-2.80 (m, 2H). |
| 41 | MS: m/z 427.0 [M + H]⁺; ¹H NMR (400 MHz, DMSO) δ(ppm), 11.31(s, 1H), |
| | 8.51(s, 2H), 7.64(d, J = 7.6 Hz, 1H), 7.31 (d, J = 10.0 Hz, 1H), 7.18-7.14(m, 4H), |
| | 7.02(s, 1H), 4.82-4.75(m, 1H), 3.21-3.11(m, 1H), 2.85-2.76(m, 2H), 2.26(s, 3H). |
| 42 | MS: 407.0 [M + H]⁺; ¹H NMR (400 MHz, DMSO) δ 11.29 (s, 1H), 8.28 (d, J = 4.9 |
| | Hz, 1H), 7.63 (d, J = 7.4 Hz, 1H), 7.30 (d, J = 10.1 Hz, 1H), 7.23-7.02 (m, 5H), |
| | 6.56 (d, J = 5.0 Hz, 1H), 5.09-4.61 (m, 1H), 3.19-3.05 (m, 1H), 2.77 (dt, J = |
| | 5.5, 3.5 Hz, 2H), 2.33 (s, 3H), 2.26 (s, 3H). |
| 43 | MS: m/z 427.0 [M + H]⁺; ¹H NMR (DMSO, 400 MHz), δ 11.31 (s, 1H), 8.41 (d, J = |
| | 5.2 Hz, 1H), 7.66 (d, J = 7.6 Hz, 1H), 7.32 (d, J = 10.0 Hz, 1H), 7.19-7.15 (m, 4H), 7.00 |
| | (br, 1H), 6.81 (d, J = 4.8 Hz, 1H), 4.80 (br, 1H), 3.23-3.16 (m, 1H), 2.87-2.74 (m, |
| | 2H), 2.27 (s, 3H) |
| 44 | MS: m/z 418.0 [M + H]⁺; ¹H NMR (400 MHz, DMSO) δ 11.37 (s, 1H), 8.74 (d, J = |
| | 2.0 Hz, 1H), 7.66 (d, J = 8.0 Hz, 1H), 7.33 (d, J = 12.0 Hz, 1H), 7.23 (d, J = 4.0 Hz, |
| | 1H), 7.20-7.15(m, 4H), 7.00 (s, 1H), 4.81 (s, 1H), 3.26-3.19(m, 1H), 2.89- |
| | 2.77(m, 2H), 2.27 (s, 3H). |
| 45 | MS: m/z 419.0 [M + H]⁺; ¹H NMR (400 MHz, DMSO) δ(ppm), 11.29(s, 1H), |
| | 8.43(d, J = 4.8 Hz, 2H), 7.63(d, J = 7.2 Hz, 1H), 7.30 (d, J = 10.0 Hz, 1H), 7.16 |
| | (d, J = 8.4 Hz, 2H), 7.09(s, 1H), 7.03 (d, J = 8.4 Hz, 2H), 6.67 (d, J = 4.8 Hz, 1H), 4.92-4.82 |
| | (m, 1H), 3.19-3.07(m, 1H), 2.83-2.76(m, 2H), 1.90-1.82(m, 1H), 0.95-0.87 (m, 2H), |
| | 0.65-0.57(m, 2H). |
| 46 | MS: m/z 445.0 [M + H]⁺; ¹H NMR (DMSO, 400 MHz), δ 11.30(s, 1H), 7.67 |
| | (d, J = 7.2 Hz, 1H), 7.35-7.31(m, 3H), 7.22-7.17(m, 2H), 7.05(br, 1H), 6.74(s, 1H), |
| | 4.82 (br, 1H), 3.19-3.12(m, 1H), 2.87-2.74(m, 2H), 2.735(s, 3H). |
| 47 | MS: m/z 416.0 [M + H]⁺; ¹H NMR (400 MHz, DMSO) δ 11.31 (s, 1H), 8.56 (s, |
| | 2H), 8.21 (s, 1H), 7.90-7.85(m, 1H), 7.68 (d, J = 8.0 Hz, 1H), 7.36 (d, J = 12.0 Hz, |
| | 1H), 7.17 (dd, J = 8.0, 4.0 Hz, 1H), 7.00 (s, 1H), 4.85 (dd, J = 12.0, 4.0 Hz, 1H), |
| | 3.28-3.21(m, 1H), 2.88-2.77(m, 2H) |
| 48 | MS: m/z 414.0 [M + H]⁺; ¹H NMR (400 MHz, DMSO) δ 11.52 (s, 1H), 11.32 (s, |
| | 1H), 8.54 (s, 2H), 7.64 (d, J = 8.0 Hz, 1H), 7.44 (dd, J = 8.0, 2.0 Hz, 1H), 7.34 (d, J = |
| | 12.0 Hz, 1H), 6.93 (s, 1H), 6.76 (s, 1H), 6.32 (d, J = 8.0 Hz, 1H), 4.79 (dd, J = 12.0, |
| | 4.0 Hz, 1H), 3.24-3.17(m, 1H), 2.89-2.67(m, 2H). |
| 49 | MS: m/z 431.0 [M + H]⁺; ¹H NMR (400 MHz, DMSO-d₆) δ 11.53 (s, 1H), 8.53 (s, |
| | 2H), 7.36-7.33 (m, 2H), 7.21-7.12 (m, 4H), 7.06 (s, 1H), 4.84-4.79 (m, 1H), 3.24- |
| | 3.17 (m, 1H), 2.98-2.96 (m, 2H). |
| 50 | MS: m/z 439.0 [M + H]⁺; ¹H NMR (400 MHz, DMSO) δ 11.31 (s, 1H), 8.53 (s, |
| | 2H), 7.64 (d, J = 8.0 Hz, 1H), 7.30 (d, J = 8.0 Hz, 1H), 7.22 (s, 4H), 7.01 (s, 1H), |
| | 4.78 (d, J = 12.5 Hz, 1H), 3.24-3.17 (m, 1H), 2.90-2.79 (m, 3H), 1.16 (d, J = 8.0 |
| | Hz, 6H). |
| 51 | MS: m/z 415.0 [M + H]⁺; ¹H NMR (DMSO-d₆, 400 MHz), δ 11.52 (s, 1H), 8.55 (s, |
| | 2H), 7.37-7.31 (m, 2H), 7.21-7.11 (m, 4H), 7.04(s, 1H), 4.78 (d, J = 12 Hz 1H), |
| | 3.24-3.16((m, 2H m, 1H), 3.02-2.91). |
| 52 | MS: m/z 438.0 [M + H]⁺; ¹H NMR (DMSO-d₆, 400 MHz), δ 11.56 (s, 1H), 8.54 (s, |
| | 2H), 7.84 (d, J = 8.4 Hz, 2H), 7.51 (d, J = 8 Hz, 2H), 7.22-7.13 (m, 2H), 7.10 (s, |
| | 1H), 4.87-4.80 (m, 1H), 3.24-3.15 (m, 1H), 3.02-2.95 (m, 2H) |
| 54 | MS: m/z 445.0 [M + H]⁺; ¹H NMR (400 MHz, DMSO-d₆) δ 11.32 (s, 1H), 8.75 (s, |
| | 1H), 7.67 (d, J = 7.2 Hz, 1H), 7.32 (d, J = 10 Hz, 1H), 7.23-7.22 (m, 5H), 7.09-6.94 |
| | (m, 1H), 4.91-4.75 (m, 1H), 3.28-3.21 (m, 1H), 2.89-2.79 (m, 3H), 1.16 (d, J = 6.8 |
| | Hz, 6H). |
| 55 | MS: m/z 446.0 [M + H]⁺; ¹H NMR (400 MHz, DMSO-d₆) δ 11.32 (s, 1H), 8.75 (s, |
| | 1H), 7.67 (d, J = 7.2 Hz, 1H), 7.32 (d, J = 10 Hz, 1H), 7.23-7.22 (m, 5H), 7.09-6.94 |
| | (m, 1H), 4.91-4.75 (m, 1H), 3.28-3.21 (m, 1H), 2.89-2.79 (m, 3H), 1.16 (d, J = 6.8 |
| | Hz, 6H). |
| 56 | MS: m/z 420.0 [M + H]⁺; ¹H NMR (DMSO, 400 MHz), δ 11.55 (s, 1H), 8.56 (d, J = |
| | 0.4 Hz, 2H), 7.84 (d, J = 8.0 Hz, 2H), 7.51(d, J = 8.0 Hz, 2H), 7.21-7.13 (m, 2H), |
| | 7.08 (s, 1H), 4.82-4.78 (m, 1H), 3.23-3.16 (m, 1H), 3.04-2.92 (m, 2H) |
| 57 | MS: m/z 455.0 [M + H]⁺; ¹H (400 MHz, DMSO-d₆) δ 11.32 (s, 1H), 8.51 (s, 2H), |
| | 7.65 (d, J = 7.2 Hz, 1H), 7.31(d, J = 10 Hz, 1H), 7.22 (s, 4H), 7.03 (s, 1H), 4.83- |
| | 4.79 (m, 1H), 3.24-3.17 (m, 1H), 2.88-2.78 (m, 3H), 1.16 (d, J = 7.2 Hz, 6H). |
| 58 | MS: m/z 415.0 [M + H]⁺; ¹H (400 MHz, DMSO) δ 11.35 (s, 1H), 8.55 (s, 2H), 7.66 |
| | (d, J = 8.0 Hz, 1H), 7.43-7.32 (m, 2H), 7.19-7.14 (m, 2H), 7.09 (dd, J = 8.0, 2.0 Hz, |
| | 1H), 7.02 (s, 1H), 4.83-4.79 (m, 1H), 3.25-3.17 (m, 1H), 2.82-2.80 (m, 2H). |
| 59 | MS: m/z 438.0 [M + H]⁺; ¹H (400 MHz, DMSO) δ(ppm), 11.35 (s, 1H), 8.54 (s, |
| | 2H), 7.85-7.83 (m, 2H), 7.67 (d, J = 7.2 Hz, 1H), 7.49 (d, J = 8.0 Hz, 2H), 7.37 (d, |
| | J = 10.0 Hz, 1H), 7.08 (s, 1H), 4.85-4.81 (m, 1H), 3.19-3.12 (m, 1H), 2.83-2.79 |
| | (m, 2H). |
| 60 | MS: m/z 463.0 [M + H]⁺; ¹H (DMSO-d₆, 400 MHz), δ 11.52 (s, 1H), 8.80 (d, J = |
| | 4.8 Hz, 1H), 7.40-7.35 (m, 2H), 7.23-7.05 (m, 6H), 4.93-4.86 (m, 1H), 3.34-3.24 |
| | (m, 1H), 3.06-2.95 (m, 2H). |
| 61 | MS: m/z 418.0 [M + H]⁺; ¹H (400 MHz, DMSO-d₆) δ 11.34 (s, 1H), 8.31 (d, J = 4.0 |
| | Hz, 1H), 7.83 (d, J = 8.4 Hz, 2H), 7.66 (d, J = 7.2 Hz, 1H), 7.49 (d, J = 8.4 Hz, 2H), |
| | 7.36 (d, J = 10 Hz, 1H), 7.20 (s, 1H), 6.62 (d, J = 4.8 Hz, 1H), 4.94-4.90 (m, 1H), |
| | 3.13-3.05 (m, 1H), 2.81-2.78 (m, 2H), 2.34 (s, 3H). |
| 62 | MS: m/z 479.0 [M + H]⁺; ¹H (400 MHz, DMSO-d₆) δ 11.55 (s, 1H), 8.55 (s, 2H), |
| | 7.74 (d, J = 8.0 Hz, 2H), 7.54 (d, J = 8.0 Hz, 2H), 7.21-7.13(m, 3H), 4.86-4.83 (m, |
| | 1H), 3.25-3.18 (m, 1H), 3.01-2.98 (m, 2H) |
| 63 | MS: m/z 429.0 [M + H]⁺; ¹H (400 MHz, DMSO-d₆) δ 11.2 (s, 1H), 8.29 (d, J = 4.8 |
| | Hz, 1H), 7.65 (d, J = 7.2 Hz, 1H), 7.43-7.37(m, 2H), 7.33 (d, J = 10.0 Hz, 1H), 7.16- |
| | 7.11 (m, 1H), 6.86-6.82 (m, 1H), 6.61 (d, J = 4.8 Hz, 1H), 4.91-4.87 (m, 1H), 3.29- |
| | 3.21 (m, 1H), 2.81-2.77 (m, 2H), 2.32 (s, 3H). |
| 64 | MS: m/z 429.0 [M + H]⁺; ¹H (400 MHz, DMSO) δ 11.37 (s, 1H), 8.77 (d, J = 8.0 Hz, |
| | 1H), 7.85 (d, J = 8.0 Hz, 2H), 7.69 (d, J = 8.0 Hz, 1H), 7.50 (d, J = 8.0 Hz, 2H), 7.39 |
| | (d, J = 12.0 Hz, 1H), 7.28 (d, J = 4.0 Hz, 1H), 7.08 (s, 1H), 4.93-4.78(m, 1H), 3.34- |
| | 3.17(m, 1H), 2.90-2.79 (m, 2H). |
| 65 | MS: m/z 463.0 [M + H]⁺; ¹H (400 MHz, DMSO) δ 11.38 (s, 1H), 8.57 (s, 2H), |
| | 7.72-7.57(m, 5H), 7.38 (d, J = 12.0 Hz, 1H), 7.09 (s, 1H), 4.83-4.79(m, 1H), 3.22- |
| | 3.13(m, 1H), 2.89-2.73(m, 2H). |
| 66 | MS: m/z 421.0 [M + H]⁺; ¹H (DMSO-d₆, 400 MHz), δ 11.28 (s, 1H), 7.62 (d, J = 8 |
| | Hz, 1H), 7.29 (d, J = 12 Hz, 1H), 7.12-7.18 (m, 5H), 6.44(s, 1H), 4.93-4.88 (m, 1H), |
| | 3.13-3.06 (m, 1H), 2.77-2.75 (m, 2H), 2.27(d, J = 6 Hz, 9 H) |
| 67 | MS: m/z 465.0 [M + H]⁺; ¹H (DMSO-d6, 400 MHz), δ 11.54(s, 1H), 8.57(s, 2H), |
| | 7.74(d, J = 8 Hz, 2H), 7.54(d, J = 8 Hz, 2H), 7.21-7.11(m, 3H), 4.84-4.79(m, 1H), |
| | 3.26-3.18(m, 1H), 3.05-2.93(m, 2H). |
| 68 | MS: m/z 438.0 [M + H]⁺; ¹H (400 MHz, DMSO-d₆) δ 11.23 (s, 1H), 8.76 (d, J = 4.8 |
| | Hz, 1H), 7.68 (d, J = 7.2 Hz, 1H), 7.47-7.40(m, 1H), 7.36 (d, J = 10.4 Hz, 1H), 7.31- |
| | 7.16 (m, 2H), 7.18-7.13 (m, 1H), 6.87-6.84 (m, 1H), 4.89-4.80 (m, 1H), 3.31-3.29 |
| | (m, 1H), 2.91-2.79 (m, 2H). |
| 69 | MS: m/z 480.0 [M + H]⁺; ¹H (400 MHz, DMSO-d₆) δ 11.34 (s, 1H), 8.30 (d, |
| | J = 5.2 Hz, 1H), 7.85 (d, J = 8.0 Hz, 2H), 7.69 (d, J = 7.2 Hz, 1H), 7.50-7.48 (m, 2H), |
| | 7.38 (d, J = 10.0 Hz, 1H), 7.06-6.99 (m, 2H), 4.86-4.77 (m, 1H), 3.21-3.14 (m, |
| | 1H), 2.89-2.79 (m, 2H). |
| 70 | MS: m/z 445.0 [M + H]⁺; ¹H (400 MHz, DMSO-d₆) δ 11.51 (s, 1H), 8.38 (d, J = 1.6 |
| | Hz, 1H), 7.43-7.41 (m, 2H), 7.33-7.31 (m, 2H), 7.18-7.11 (m, 2H), 7.04 (s, 1H), |
| | 4.81-4.77 (m, 1H), 3.20-3.13 (m, 1H), 2.98-2.94 (m, 2H), 2.37 (d, 2.4 Hz, 3H). |
| 71 | MS: m/z 405.0 [M + H]⁺; ¹H (400 MHz, DMSO) δ 11.25 (s, 1H), 8.54 (s, 2H), 7.51 |
| | (d, J = 2.0 Hz, 1H), 7.44 (d, J = 8.0 Hz, 2H), 7.33-7.26(m, 3H), 7.07 (dd, J = 8.0, |
| | 2.0 Hz, 1H), 7.04 (s, 1H), 6.71 (dd, J = 16.0, 8.0 Hz, 1H), 5.80 (d, J = 17.7 Hz, |
| | 1H), 5.25 (d, J = 20.0 Hz, 1H), 4.79 (d, J = 12.0 Hz, 1H), 3.24-3.17 (m, 1H), 2.81- |
| | 2.79 (m, 2H). |
| 72 | MS: m/z 461.0 [M + H]⁺; ¹H (DMSO-d₆, 400 MHz), δ 11.308 (s, 1H), 8.77(br, 1H), |
| | 7.66 (d, J = 4 Hz, 1H), 7.31 (d, J = 10 Hz, 1H), 7.21-7.14 (m, 4 H), 7.09-7.10 (d, J = 4 |
| | Hz, 1H), 7.02(br, 1H), 4.88(br, 1H), 3.2-3.29 (m, 1H), 2.76-2.9 (m, 2H), 2.26 (s, 3 H). |
| 73 | MS: m/z 481.0 [M + H]⁺; ¹H (DMSO-d₆, 400 MHz), δ 11.52 (s, 1H), 8.795(d, J = 4 |
| | Hz, 1H), 7.44 (d, J = 4 Hz, 2H), 7.35 (d, J = 8 Hz, 2H), 7.13-7.19 (m, 3H), 7.08 (br, |
| | 1H), 4.9(br, 1H), 3.31-3.22 (m, 1H), 3.02 (m, 2H), |
| 74 | MS: m/z 515.0 [M + H]⁺; ¹H (DMSO-d₆, 400 MHz), δ 11.54 (br, 1H), 8.81 (d, J = 4.8 |
| | Hz, 1H), 7.75 (d, J = 8 Hz, 2H), 7.57 (d, J = 8 Hz, 2H), 7.21-7.13 (m, 4 H), 4.92 (br, |
| | 1H), 3.3-3.22 (m, 1H), 3.04 (s, 2H) |
| 75 | MS: m/z 513.0 [M + H]⁺; ¹H (400 MHz, DMSO) δ(ppm), 11.33(s, 1H), 8.81 |
| | (d, J = 4.8 Hz, 1H), 7.75(d, J = 8.0 Hz, 2H), 7.70(d, J = 7.2 Hz, 1H), 7.55(d, J = 8.0 Hz, 2H), |
| | 7.37(d, J = 10.0 Hz, 1H), 7.19-7.09(m, 2H), 5.00-4.84(m, 1H), 3.30-3.22(m, 1H), 2.95- |
| | 2.78(m, 2H). |
| 76 | MS: m/z 461.0 [M + H]⁺; ¹H (400 MHz, DMSO-d₆) δ 11.51 (s, 1H), 8.78 (s, 1H), |
| | 7.23-7.21 (m, 2H), 7.17-7.15 (m, 4H), 7.13-7.01 (m, 2H), 4.90-4.83 (m, 1H), |
| | 3.30-3.26 (m, 1H), 3.04-2.94 (m, 2H), 2.27 (s, 3H). |
| 77 | MS: m/z 449.0 [M + H]⁺; ¹H (400 MHz, DMSO-d₆) δ 11.23 (s, 1H), 8.42 (d, |
| | J = 5.2 Hz, 1H), 7.68 (d, J = 7.6 Hz, 1H), 7.46-7.42 (m, 1H), 7.35 (d, J = 10.0 Hz, |
| | 1H), 7.28 (s, 1H), 7.18-7.13 (m, 1H), 6.87-6.83 (m, 2H), 4.83-4.79 (m, 1H), 3.31- |
| | 3.27 (m, 1H), 2.85-2.81 (m, 2H). |
| 78 | MS: m/z 427.0 [M + H]⁺; ¹H (400 MHz, DMSO) δ 11.31 (s, 1H), 8.53 (s, 2H), 7.64 |
| | (d, J = 8.0 Hz, 1H), 7.32-7.23(m, 5H), 7.02 (s, 1H), 5.15 (t, J = 4.0 Hz, 1H), 4.78 (d, |
| | J = 20.0 Hz, 1H), 4.46 (d, J = 4.0 Hz, 2H), 3.23-3.16(m, 1H), 2.81-2.77(m, 2H). |
| 79 | MS: m/z 421.0 [M + H]⁺; ¹H (400 MHz, DMSO) δ 11.33 (s, 1H), 8.54 (s, 2H), 7.65 |
| | (d, J = 8.0 Hz, 1H), 7.44 (d, J = 8.0 Hz, 2H), 7.33 (d, J = 12.0 Hz, 1H), 7.27 (d, J = 8.0 |
| | Hz, 2H), 7.02 (s, 1H), 6.71 (dd, J = 20.0, 12.0 Hz, 1H), 5.80 (d, J = 16.0 Hz, 1H), |
| | 5.25 (d, J = 12.0 Hz, 1H), 4.79 (d, J = 16.0 Hz, 1H), 3.23-3.16(m, 1H), 2.86-2.76(m, 2H). |
| 80 | MS: m/z 481.0 [M + H]⁺; ¹H (DMSO-d₆, 400 MHz), δ 11.35 (s, 1H), 8.54(s, 2H), |
| | 7.73 (d, J = 8.4 Hz, 2H), 7.68 (d, J = 7.6 Hz, 1H), 7.52 (, J = 8 Hz, 2H), 7.37 (d, J = 10 |
| | Hz, 1H), 7.11(s, 1H), 4.86-4.82(m, 1H), 3.22-3.15 (m, 1H), 2.84-2.8 (m, 2H) |
| 81 | MS: m/z 461.0 [M + H]⁺; ¹H (DMSO-d₆, 400 MHz), δ 11.36 (s, 1H), 8.313 (d, J = 4.8 |
| | Hz, 1H), 7.73 (d, J = 8 Hz, 2H), 7.66 (d, J = 7.2 Hz, 1H), 7.54 (d, J = 8 Hz, 2H), 7.36 |
| | (d, J = 10.4 Hz, 1H), 7.23(s, 1H), 6.61(d, J = 4.8 Hz, 1H), 4.95-4.92 (m, 1H), 3.16- |
| | 3.09 (m, 1H), 2.82-2.80 (m, 2H), 2.35 (s, 3 H) |
| 82 | MS: m/z 395.0 [M + H]⁺; ¹H (400 MHz, DMSO) δ(ppm), 11.38(s, 1H), 8.53(s, 2H), |
| | 7.20-7.14(m, 4H), 7.11-7.04(m, 2H), 7.02(s, 1H), 4.76(dd, J = 12.8, 3.6 Hz, 1H), 3.25- |
| | 3.18(m, 1H), 3.02-2.87(m, 2H), 2.27 (s, 3H) |
| 83 | MS: m/z 399.0 [M + H]⁺; ¹H (400 MHz, DMSO) δ(ppm), 11.40(s, 1H), 8.55 (s, 2H), |
| | 7.36-7.32(m, 2H), 7.20-7.16 (m, 2H), 7.10-7.04(m, 3H), 4.82-4.73(m, 1H), 3.24- |
| | 3.17(m, 1H), 3.03-2.89(m, 2H). |
| 84 | MS: m/z 411.0 [M + H]⁺; ¹H (400 MHz, DMSO) δ(ppm), 11.39(s, 1H), |
| | 8.51(s, 2H), 7.20-7.14(m, 4H), 7.09-7.03(m, 3H), 4.83-4.74(m, 1H), 3.25-3.16(m, 1H), |
| | 3.02-2.91(m, 2H), 2.27(s, 3H). |
| 85 | MS: m/z 413.0 [M + H]⁺; ¹H (400 MHz, DMSO) δ(ppm), 11.41(s, 1H), |
| | 8.53(s, 2H), 7.36-7.32(m, 2H), 7.21-7.16(m, 2H), 7.13-7.04(m, 3H), 4.85-4.76(m, 1H), |
| | 3.25-3.14(m, 1H), 3.03-2.92(m, 2H). |
| 86 | MS: m/z 413.0 [M + H]⁺; ¹H (400 MHz, DMSO) δ 11.32 (s, 1H), 8.53 (s, 2H), 7.64 |
| | (d, J = 8.0 Hz, 1H), 7.30 (d, J = 12.0 Hz, 1H), 7.21-7.13(m, 4H), 7.01 (s, 1H), 4.81- |
| | 4.74(m, 1H), 4.64 (t, J = 4.0 Hz, 1H), 3.58-3.53(m, 2H), 3.21-3.16(m, 1H), 2.80- |
| | 2.77(m, 2H), 2.70-2.66(m, 2H). |
| 87 | MS: m/z 471.0 [M + H]⁺; ¹H (DMSO-d₆, 400 MHz), 811.54(s, 1H), 8.71(d, J = 4.8 Hz, |
| | 1H), 7.44-7.37(m, 4H), 7.21-7.12(m, 4H), 4.96-4.92(m, 1H), 3.91(s, 3H), 3.32- |
| | 3.20(m, 1H), 3.01-2.98(m, 2H). |
| 88 | MS: m/z 486.0 [M + H]⁺; ¹H (400 MHz, DMSO) δ(ppm), 11.32(s, 1H), |
| | 7.84(d, J = 8.4 Hz, 2H), 7.69(d, J = 7.2 Hz, 1H), 7.50(d, J = 8.0 Hz, 2H), |
| | 7.37(d, J = 10.0 Hz, 1H), 7.20-7.00(m, 2H), 5.07-4.76(m, 1H), 3.25-3.15(m, 1H), 2.95- |
| | 2.72(m, 2H), 2.48(s, 3H). |
| 89 | MS: m/z 470.0 [M + H]⁺; ¹H (DMSO, 400 MHz), δ11.54(s, 1H), 8.81(d, J = 4.8 Hz, 1H), |
| | 7.86(d, J = 8.4 Hz, 2H), 7.53(d, J = 8.4 Hz, 2H), 7.22-7.12(m, 4H), 4.93-4.88(m, 1H), |
| | 3.33-3.25(m, 1H), 3.07-3.00(m, 2H) |
| 90 | MS: m/z 411.0 [M + H]⁺; ¹H (DMSO-d₆, 400 MHz), δ 11.51 (s, 1H), 8.29 (d, J = 4.8 |
| | Hz, 1H), 7.37-7.33 (m, 2H), 7.20-7.11 (m, 5H), 6.59(d, J = 5.2 Hz, 1H), 4.91-4.88 |
| | (m, 1H), 3.18-3.11 (m, 1H), 2.96-2.93 (m, 2H), 2.34 (s, 3 H) |
| 93 | MS: m/z 432.0 [M + H]⁺; ¹H NMR (400 MHz, DMSO-d₆) δ 11.32 (s, 1H), 8.82 (d, |
| | J = 8.4 Hz, 2H), 7.66 (d, J = 7.2 Hz, 1H), 7.48 (d, J = 8.4 Hz, 2H), 7.35 (d, J = 10.0 |
| | Hz, 1H), 7.22 (s, 1H), 6.90 (s, 1H), 4.96-4.92 (m, 1H), 3.10-3.03 (m, 1H), 2.82- |
| | 2.77 (m, 2H), 2.29 (s, 6H) |
| 97 | MS: m/z 461.0 [M + H]⁺; ¹H NMR (400 MHz, DMSO) δ(ppm), 11.35(s, 1H), |
| | 8.81(s, 2H), 7.66(d, J = 7.2 Hz, 1H), 7.33(d, J = 10.0 Hz, 1H), 7.20-7.15(m, 5H), 4.93 |
| | (dd, J = 13.8, 4.8 Hz, 1H), 3.27-3.20(m, 1H), 2.90-2.76(m, 2H), 2.27(s, 3H). |
| 98 | MS: m/z 462.9 [M − H]⁻; ¹H NMR (400 MHz, DMSO) δ(ppm), 11.36(s, 1H), |
| | 8.82(s, 2H), 7.68(d, J = 7.6 Hz, 1H), 7.36-7.33(m, 3H), 7.22-7.17(m, 3H), 4.95 |
| | (dd, J = 13.2, 4.8 Hz, 1H), 3.26-3.19(m, 1H), 2.91-2.77(m, 2H). |
| 119 | MS: m/z 465.0 [M + H]⁺; ¹H NMR (400 MHz, DMSO-d₆) δ 11.17 (s, 1H), 8.79 (d, |
| | J = 4.8 Hz, 1H), 7.55 (d, J = 2.0 Hz, 1H), 7.45-7.38 (m, 1H), 7.36-7.27 (m, 2H), |
| | 7.17-7.11 (m, 2H), 7.09 (dd, J = 8.4, 2.0 Hz, 1H), 6.88 (t, J = 6.8 Hz, 1H), 4.91- |
| | 4.88 (m, 1H), 3.42-3.36 (m, 1H), 2.93-2.83 (m, 2H). |
| 120 | MS: m/z 465.0 [M + H]⁺; ¹H NMR (400 MHz, DMSO-d₆) δ 11.15 (s, 1H), 8.78 (d, |
| | J = 4.8 Hz, 1H), 7.54 (d, J = 2.0 Hz, 1H), 7.36-7.22 (m, 2H), 7.13 (d, J = 4.8 |
| | Hz, 1H), 7.08 (dd, J = 8.8, 2.4 Hz, 1H), 6.94 (t, J = 7.6 Hz, 1H), 6.77 (t, J = 8.4 Hz, |
| | 1H), 4.98-4.78 (m, 1H), 3.83 (s, 3H), 3.43-3.36 (m, 1H), 2.92-2.78 (m, 2H). |
| 176 | MS: m/z 433.9, [M − H]⁻; ¹H NMR (400 MHz, CDCl₃) δ 8.51 (d, J = 4.8 Hz, 1H), |
| | 8.00 (s, 1H), 7.50 (d, J = 2.0 Hz, 1H), 7.24-7.22 (m, 2H), 7.15-7.12 (m, 1H), |
| | 6.87-6.82 (m, 2H), 6.79-6.76 (m, 1H), 5.16-5.12 (m, 1H), 3.52-3.45 (m, 1H), |
| | 2.99-2.83 (m, 2H), 2.28 (d, J = 1.2 Hz, 3H). |
| 179 | MS: m/z 429.2 [M + H]⁺; ¹H NMR (400 MHz, DMSO-d₆) 11.13 (s, 1H), 8.55 (s, |
| | 2H), 7.51 (d, J = 2.0 Hz, 1H), 7.31(d, J = 8.4 Hz, 1H), 7.28 (s, 1H), 7.09-7.08 (m, |
| | 1H), 7.02 (t, J = 7.6 Hz, 1H), 6.68 (t, J = 7.2 Hz, 1H), 4.78-4.74 (m, 1H), 3.33- |
| | 3.25 (m, 1H), 2.82-2.79 (m, 2H), 2.26 (s, 3H). |
| 180 | MS: m/z 477.0 [M − H]⁻; ¹H NMR (400 MHz, DMSO-d₆) 11.16 (s, 1H), 8.79 (d, J = |
| | 4.8 Hz, 1H), 7.54 (d, J = 1.6 Hz, 1H), 7.33-7.27 (m, 2H), 7.14 (d, J = 4.8 Hz, 1H), |
| | 7.08 (dd, J = 8.4, 2.0 Hz, 1H), 7.03(t, J = 7.2 Hz, 1H), 6.76 (t, J = 7.0 Hz, |
| | 1H), 4.91-4.88 (m, 1H), 3.42-3.33 (m, 1H), 2.93-2.78 (m, 2H), 2.09 (s, 3H). |
| 185 | MS: m/z 425.1 [M + H]⁺; ¹H NMR (400 MHz, DMSO-d₆) δ 11.12 (s, 1H), 8.29 (d, |
| | J = 4.0 Hz, 1H), 7.51 (d, J = 2.0 Hz, 1H), 7.39 (s, 1H), 7.31 (d, ] = 8.0 Hz, 1H), |
| | 7.07 (dd, J = 8.0, 2.0 Hz, 1H), 7.01 (t, J = 4.0 Hz, 1H), 6.72 (t, J = 4.0 Hz, 1H), |
| | 6.60 (d, J = 8.0 Hz, 1H), 4.91-4.87 (m, 1H), 3.35-3.20 (m, 1H), 2.81-2.79 (m, |
| | 2H), 2.32 (s, 3H), 2.25 (d, J = 2.0 Hz, 3H). |
| 187 | MS: m/z 443.2 [M + H]⁺; ¹H NMR (400 MHz, DMSO-d₆) δ 11.11 (s, 1H), 8.37 (d, |
| | J = 1.2 Hz, 1H), 7.51 (d, J = 1.2 Hz, 1H), 7.30 (d, J = 8.4 Hz, 1H), 7.26 (s, 1H), 7.07 |
| | (dd, J = 8.8, 2.0 Hz, 1H), 7.01 (t, J = 7.2 Hz, 1H), 6.69 (t, J = 7.2 Hz, 1H), 4.79- |
| | 4.76 (m, 1H), 3.29-3.26 (m, 1H), 2.85-2.74 (m, 2H), 2.35 (d, J = 2.0 Hz, 3H), |
| | 2.25 (s, 3H). |
| 206 | ESI-MS: m/z 509.0 [M − H]⁻; ¹H NMR (400 MHz, DMSO-d₆) δ: 11.41 (s, 1H), 7.26 |
| | (s, 1H), 7.17-7.11 (m, 2H), 7.06-7.01 (m, 2H), 6.84-6.80 (m, 1H), 4.99-4.82 (m, |
| | 1H), 3.45-3.37 (m, 1H), 3.05-2.94 (m, 2H), 2.46 (s, 3H), 2.26-2.25 (m, 3H). |
| 218 | MS: m/z 477.0 [M − H]⁻; ¹H NMR (400 MHz, DMSO) δ: 11.28 (s, 1H), 10.15 (s, |
| | 1H), 8.80 (d, J = 4.0 Hz, 1H), 7.54 (d, J = 2.0 Hz, 1H), 7.33 (dd, J = 12.0, 8.0 Hz, 2H), |
| | 7.15-6.96 (m, 3H), 6.88 (s, 1H), 6.80 (d, J = 8.0 Hz, 1H), 4.88 (s, 1H), 3.34- |
| | 3.19(m, 1H), 2.88-2.50 (m, 2H). |
| 219 | MS: m/z 427.0 [M − H]⁻; ¹H NMR (400 MHz, DMSO) δ 11.27 (s, 1H), 10.13 (s, |
| | 1H), 8.55 (s, 2H), 7.52 (d, J = 4.0 Hz, 1H), 7.32 (dd, J = 16.0, 12.0 Hz, 2H), 7.08 |
| | (dd, J = 8.0, 2.0 Hz, 1H), 6.99 (s, 1H), 6.90 (d, J = 2.0 Hz, 1H), 6.76 (dd, J = 8.0, |
| | 4.0 Hz, 1H), 4.79-4.75 (m, 1H), 3.16-3.13 (m, 1H), 2.86-2.76 (m, 2H). |
| 221 | MS: m/z 427.0 [M − H]⁻; ¹H NMR (400 MHz, DMSO) δ 10.96 (s, 1H), 10.35 (s, |
| | 1H), 8.53 (s, 2H), 7.47 (d, J = 2.0 Hz, 1H), 7.30 (d, J = 8.4 Hz, 1H), 7.11 (s, 1H), |
| | 7.04 (dd, J = 8.8, 2.4 Hz, 1H), 6.90 (d, J = 2.0 Hz, 1H), 6.78 (dd, J = 8.4, 2.0 Hz, |
| | 1H), 6.72 (d, J = 8.4 Hz, 1H), 4.79-4.69 (m, 1H), 3.29-3.22 (m, 1H), 2.81- |
| | 2.72 (m, 2H). |
| 222 | MS: m/z 477.0 [M − H]⁻; ¹H NMR (400 MHz, DMSO) δ 11.05 (s, 1H), 10.35- |
| | 10.25 (m, 1H), 8.75 (d, J = 4.8 Hz, 1H), 7.50 (d, J = 2.0 Hz, 1H), 7.31 (d, J = 8.4 |
| | Hz, 1H), 7.25 (s, 1H), 7.09 (d, J = 4.8 Hz, 1H), 7.06 (dd, J = 8.4, 2.0 Hz, 1H), 6.88 |
| | (d, J = 1.6 Hz, 1H), 6.78 (dd, J = 8.0, 2.0 Hz, 1H), 6.73 (d, J = 8.0 Hz, 1H), 4.88- |
| | 4.78 (m, 1H), 3.37-3.28 (m, 1H), 2.86-2.76 (m, 2H). |
| 235 | MS: m/z 443.0 [M + H]⁺; ¹H NMR (400 MHz, DMSO) δ 11.11 (s, 1H), 8.36 (d, J = |
| | 1.6 Hz, 1H), 7.51 (d, J = 2.0 Hz, 1H), 7.30 (d, J = 8.4 Hz, 1H), 7.26 (s, 1H), 7.06 |
| | (dd, J = 8.4, 2.0 Hz, 1H), 7.01 (t, J = 7.4 Hz, 1H), 6.70 (t, J = 7.2 Hz, 1H), 4.82- |
| | 4.73 (m, 1H), 3.32-3.24 (m, 1H), 2.83-2.76 (m, 2H), 2.35 (d, J = 2.4 Hz, 1H), |
| | 2.25 (s, 3H). |
| 252 | MS: m/z 462.8 [M + H]⁺; ¹H NMR (DMSO-d₆, 400 MHz) δ: 11.05(s, 1H), 9.99 |
| | (brs, 1H), 8.79-8.78(d, J = 4.8 Hz, 1H), 7.51-7.50(d, J = 2.0 Hz, 1H), 7.32-7.29 |
| | (d, J = 8.8 Hz, 1H), 7.25(s, 1H), 7.16-7.11(m, 2H), 7.07-7.04(dd, J = 2.0 Hz, 8.4 Hz, 1H), |
| | 6.76-6.71 (m, 1H), 6.60-6.58(d, J = 8.0 Hz, 1H), 4.85-4.82(d, J = 11.6 Hz, 1H), 3.41- |
| | 3.35(m, 1H), 2.84-2.80(m, 2H). |
| 254 | MS: m/z 459 [M + H]⁺; (DMSO-d, 400 MHz) 11.17(s, 1H), 8.78(d, J = 4.4 Hz, 1H), |
| | 7.53(d, J = 2.0 Hz, 1H), 7.31 (d, J = 8.4 Hz, 2H), 7.12 (d, J = 5.2 Hz, 1H), 7.09 (d, |
| | J = 2.0 Hz, 1H), 7.07 (d, J = 2.0 Hz, 1H), 6.95 (d, J = 8.4 Hz, 1H), 6.89 (t, J = 8.0 Hz, |
| | 1H), 4.85 (t, J = 7.2 Hz, 1H), 3.35 (s, 1H), 2.85 (q, J = 6.4 Hz, 2H), 2.30 (s, 3H). |
| 255 | MS: m/z 472 [M + H]⁺; (CDCl₃-d, 400 MHz) δ: 8.58 (d, J = 4.8 Hz, 1H), 8.01 (s, 1H), |
| | 7.54-7.53 (m, 1H), 7.47-7.45 (m, 1H), 7.42-7.35 (m, 2H), 7.27-7.25 (m, 2H), 7.19- |
| | 7.16 (m, 1H), 6.88 (d, J = 4.8 Hz, 1H), 5.25-5.21 (m, 1H), 3.60-3.53 (m, 1H), 3.05- |
| | 2.89 (m, 2H). |
| 256 | MS: m/z 463 [M + H]⁺; (DMSO-d, 400 MHz) 11.19(s, 1H), 9.86(s, 1H), 8.78(d, |
| | J = 4.8 Hz, 1H), 7.52 (d, J = 2 Hz, 1H), 7.33(d, J = 8.8 Hz, 2H), 7.09(m, 2H), 6.92(m, 2H), |
| | 6.38 (m, 1H), 4.85(t, J = 9.6 Hz, 1H), 3.37(t, J = 11.2 Hz, 1H), 2.84(t, J = 3.6 Hz, 2H). |
| 257 | MS: m/z 456.9 [M − H]⁻; ¹H NMR (400 MHz, DMSO) δ 11.01 (s, 1H), 9.61 (br s, |
| | 1H), 8.74 (d, J = 4.8 Hz, 1H), 7.48 (d, J = 2.1 Hz, 1H), 7.29 (d, J = 8.6 Hz, 1H), |
| | 7.21 (s, 1H), 7.09-7.00 (m, 2H), 6.67 (s, 1H), 6.62 (d, J = 7.8 Hz, 1H), 6.56- |
| | 6.48 (m, 1H), 4.82 (d, J = 13.0 Hz, 1H), 3.38 (ddd, J = 13.6, 10.4, 5.9 Hz, 1H), |
| | 2.89-2.70 (m, 2H), 2.19 (s, 3H). |
| 258 | MS: m/z 456.9 [M − H]⁻; ¹H NMR (400 MHz, DMSO) δ 11.25 (s, 1H), 9.23 (s, 1H), |
| | 8.78 (s, 1H), 7.52 (d, J = 2.1 Hz, 1H), 7.32 (d, J = 8.6 Hz, 1H), 7.14-6.92 (m, 3H), |
| | 6.70 (s, 2H), 4.86 (br s, 1H), 3.25 (m, 2H), 2.91-2.73 (m, 2H), 2.07 (s, 3H). |
| 259 | MS: m/z 459.1 [M − H]⁻; ¹H NMR (DMSO-d, 400 MHz) δ: 11.18 (s, 1H), 8.79(d, |
| | J = 4.8 Hz, 1H), 7.53(d, J = 2.0 Hz, 1H), 7.38 (s, 1H), 7.32 (d, J = 8.4 Hz, 1H), 7.27 (t, |
| | J = 7.2 Hz, 1H), 7.13 (d, J = 4.8 Hz, 1H), 7.08 (q, J = 6.4 Hz, 1H), 7.03 (t, J = 7.6 |
| | Hz, 1H), 6.80 (t, J = 6.8 Hz, 1H), 4.84 (t, J = 3.2 Hz, 1H), 3.36 (s, 1H), 2.85 (t, J = 3.6 |
| | Hz, 2H), 2.26 (s, 3H). |
| 260 | MS: m/z 495.1 [M + H]⁺; ¹H NMR (400 MHz, DMSO) δ: 11.25 (s, 1H), 8.81 (s, |
| | 1H), 8.23 (s, 1H), 7.70 (s, 1H), 7.64 (d, J = 8.6 Hz, 2H), 7.56 (d, J = 2.0 Hz, 1H), |
| | 7.44 (d, J = 8.6 Hz, 2H), 7.34 (d, J = 8.6 Hz, 1H), 7.13 (d, J = 4.9 Hz, 1H), 7.10 (dd, |
| | J = 8.5, 2.1 Hz, 2H), 4.94 (s, 1H), 2.88 (dt, J = 21.8, 11.8 Hz, 2H). |
| 261 | MS: m/z 494.9 [M + H]⁺; ¹H NMR (400 MHz, CDCl3) δ 8.56 (d, J = 4.8 Hz, 1H), |
| | 8.05 (s, 1H), 7.88 (d, J = 2.4 Hz, 1H), 7.71 (d, J = 1.4 Hz, 1H), 7.60 (d, J = 8.6 Hz, |
| | 2H), 7.54 (d, J = 1.7 Hz, 1H), 7.49 (d, J = 8.6 Hz, 2H), 7.22 (d, J = 8.6 Hz, 1H), 7.14 |
| | (dd, J = 8.5, 2.0 Hz, 2H), 6.82 (d, J = 4.8 Hz, 1H), 6.56-6.40 (m, 1H), 5.09 (dd, J = |
| | 13.5, 4.9 Hz, 1H), 3.67-3.24 (m, 1H), 3.07-2.94 (m, 1H), 2.88 (dd, J = 15.4, 3.6 Hz, 1H). |
| 262 | MS: m/z 511.9 [M − H]⁻; ¹H NMR (400 MHz, DMSO-d₆) δ 11.18 (s, 1H), 8.76 (s, |
| | 1H), 7.52 (d, J = 2.1 Hz, 1H), 7.30 (d, J = 8.6 Hz, 1H), 7.19-7.13 (m, 2H), 7.13- |
| | 6.93 (br s overlapping, 1H), 7.08-7.04 (m overlapping, 2H), 6.94-6.84 (m, 2H), |
| | 4.88 (s, 1H), 3.74-3.62 (m, 4H), 3.31-3.21 (m, 1H), 2.94-2.73 (m, 2H). |
| 263 | MS: m/z 481 [M + H]⁺; ¹H NMR (DMSO-d, 400 MHz) 11.14(s, 1H), 8.79(d, J = 4.8 |
| | Hz, 1H), 7.55(d, J = 2.4 Hz, 1H), 7.33 (d, 1H), 7.23 (m, 2H), 7.15 (d, J = 4.8 Hz, 1H), |
| | 7.09 (q, J = 6.8 Hz, 1H), 6.92 (q, J = 7.2 Hz, 1H), 4.91 (d, J = 9.6 Hz, 1H), 3.40 (t, |
| | J = 11.6 Hz, 1H), 2.89 (m, 2H). |
| 267 | MS: m/z 481 [M + H]⁺; (DMSO, 400 MHz) 11.16(s, 1H), 8.79(d, J = 4.8 Hz, 1H), |
| | 7.54(d, J = 2 Hz, 1H), 7.50(q, J = 2 Hz, 1H), 7.31(d, J = 5.2 Hz, 2H), 7.24(q, J = 6.4 Hz, |
| | 1H), 7.14(d, J = 4.4 Hz, 1H), 7.09(q, J = 4.8 Hz, 1H), 7.04(d, J = 8 Hz, 1H), |
| | 4.87(d, J = 9.6 Hz, 1H), 3.36(d, J = 8.8 Hz, 1H), 2.87(m, 1H). |
| 268 | MS: m/z 525 [M + H]⁺; (DMSO-d, 400 MHz) 11.16 (s, 1H), 8.79 (d, J = 4.4 Hz, 1H), |
| | 7.61(q, J = 7.6 Hz, 1H), 7.54 (d, J = 2.0 Hz, 1H), 7.37 (q, J = 6.8 Hz, 1H), 7.32 (d, |
| | J = 8.4 Hz, 1H), 7.29 (s, 1H), 7.14 (d, J = 4.8 Hz, 1H), 7.09 (q, J = 6.4 Hz, 1H), 6.97 |
| | (t, J = 8.0 Hz, 1H), 4.85 (d, J = 2.0 Hz, 1H), 3.30 (s, 1H), 2.85 (q, J = 10.8 Hz, 2H). |
| 269 | MS: m/z 496 [M + H]⁺; (CDCL3, 400 MHz) 8.61 (d, J = 4.8 Hz, 1H), 8.40 (s, 1H), |
| | 8.34 (s, 2H), 7.65 (d, J = 8.0 Hz, 2H), 7.57 (d, J-1.6 Hz, 1H), 7.34-7.30 (m, 3H), |
| | 7.22-7.17 (m, 2H), 6.88 (d, J = 4.8 Hz, 1H), 5.17-5.12 (m, 1H), 3.40-3.34 (m, 1H), |
| | 3.07-2.90 (m, 2H). |
| 270 | MS m/z 540.5 [M − H]⁻. (DMSO-d₆, 400 MHz) δ: 8.49-8.47 (d, J = 4.8 Hz, 1H), |
| | 7.85 (s, 1H), 7.43-7.43 (d, J = 2.0 Hz, 1H), 7.16-7.13 (m, 3H), 7.08-7.05 (dd, J = |
| | 2.0 Hz, 8.4 Hz, 1H), 6.82-6.77 (m, 2H), 5.14-5.09 (dd, J = 4.0, 13.6 Hz, 1H), 3.46- |
| | 3.39 (ddd, J = 2.0, 4.0, 11.2 Hz, 1H), 2.93-2.78 (m, 2H). |
| 271 | MS: m/z 459 [M + H]⁺; (CDCL3-d, 400 MHz) 8.53 (d, J = 4.8 Hz, 1H), 7.79(d, J = 8.0 |
| | Hz, 1H), 7.32 (s, 1H), 7.19 (d, J = 8.4, 1H), 6.98 ((d, J = 8.0 Hz, 1H), 6.80 (t, J = 4.0 |
| | Hz, 3H), 5.15 (q, J = 13.6 Hz, 1H), 3.50 (m, 1H), 2.96 (m, 1H), 2.87 (q, J = 15.2 Hz, |
| | 1H), 2.45 (s, 3H), 2.26(d, J = 1.6 Hz, 3H). |
| 274 | MS: m/z 445 [M + H]⁺; (MeOD-d, 400 MHz) 8.42(d, J = 4.8 Hz, 2H), |
| | 8.20 (s, 1H), 7.62 (s, 1H), 7.50 (m, 3H), 7.36 (q, J = 6.4 Hz, 1H), 7.27 (d, |
| | J = 8.4 Hz, 1H), 7.22 (t, J = 8.0 Hz, 1H), 7.16 (s, 1H), 7.07 (q, J = 6.8 Hz, 1H), 6.73 |
| | (t, J = 4.8 Hz, 1H), 4.98 (m, 1H), 3.42 (m, 1H) 2.92 (m, 1H), 2.81 (m, 1H). |
| 275 | MS: m/z 513 [M + H]⁺; (MeOD-d, 400 MHz) 8.69(d, J = 4.8 Hz, 1H), 8.20(s, 1H), |
| | 7.62(t, J = 1.6 Hz, 1H), 7.51 (q, J = 8.4 Hz, 2H), 7.45 (s, 1H), 7.37 (q, J = 6.4 Hz, 1H), |
| | 7.28 (m, 2 H), 7.16 (s, 1H), 7.08 (q, J = 6.4 Hz, 1H), 6.98 (d, J = 4.8 Hz, 1H), 5.08 |
| | (q, J = 9.6 Hz, 1H), 3.51 (m, 1H) 2.93 (m, 2H) |
| 277 | MS: m/z 530.0 [M + H]⁺; (DMSO-d₆, 400 MHz) δ 11.16 (s, 1H), 8.77(d, J = 4.8 Hz, |
| | 1H), 7.52 (d, J = 2.0 Hz, 1H), 7.29 (t, J = 8.4 Hz, 2H), 7.09 (d, J = 4.8 Hz, 1H), 7.07 |
| | (q, J = 6.4 Hz, 1H), 6.80 (m, 2H), 6.68 (q, J = 6.0 Hz, 1H), 4.86 (s, 1H), 3.70 (t, |
| | J = 4.4 Hz, 4H), 3.01 (s, 1H), 3.11 (t, J = 4.8 Hz, 4H), 2.84 (d, J = 3.2 Hz, 2H). |
| 278 | MS: m/z 487.8 [M + H]⁺; (CDCl₃, 400 MHz) δ 8.57-8.56 (d, J = 4.8 Hz, 1H), 7.93 |
| | (s, 1H), 7.51-7.51 (d, J = 2.0 Hz, 1H), 7.33-7.29 (m, 2H), 7.23(s, 1H), 7.17-7.15 |
| | (dd, J = 2.0 Hz, 8.8 Hz, 1H), 7.13-7.09(t, J = 6.4 Hz, 1H), 6.89-6.88 (d, J = 4.8 Hz, 1H), 5.19- |
| | 5.15(dd, J = 5.2 Hz, 13.2 Hz, 1H), 3.57-3.50(m, 1H), 3.03-2.92(m, 2H). |
| 279 | MS: m/z 547.8 [M + H]⁺; (CDCl3, 400 MHz) δ 8.55-8.54 (d, J = 4.8 Hz, 1H), 7.94 |
| | (s, 1H), 7.49-7.49 (d, J = 2.0 Hz, 1H), 7.26-7.20 (m, 2H), 7.14-7.11 (dd, J = 2.0 |
| | Hz, 8.8 Hz, 1H), 6.84-6.81 (m, 2H), 6.58-6.54 (t, J = 7.6 Hz, 1H), 5.18-5.14 (dd, J = |
| | 4.8 Hz, 13.6 Hz, 1H), 3.85-3.83 (t, J = 4.4 Hz, 4H), 3.52-3.45 (td, J = 4.4 Hz, |
| | 12.0 Hz, 1H), 3.08-3.05 (t, J = 4.4 Hz, 4H), 2.95-2.86 (m, 2H). |
| 280 | MS: m/z 475.0 [M + H]⁺; ¹H NMR (400 MHz, DMSO) δ 11.11 (s, 1H), 8.36 (d, J = |
| | 1.6 Hz, 1H), 7.51 (d, J = 2.0 Hz, 1H), 7.30 (d, J = 8.4 Hz, 1H), 7.26 (s, 1H), 7.06 |
| | (dd, J = 8.4, 2.0 Hz, 1H), 7.01 (t, J = 7.4 Hz, 1H), 6.70 (t, J = 7.2 Hz, 1H), 4.82- |
| | 4.73 (m, 1H), 3.32-3.24 (m, 1H), 2.83-2.76 (m, 2H), 2.35 (d, J = 2.4 Hz, 1H), |
| | 2.25 (s, 3H). |
| 281 | MS: m/z 511.0 [M + H]⁺; ¹H NMR (400 MHz, DMSO) δ 11.12 (d, J = 4.0 Hz, 1H), |
| | 8.69 (t, J = 3.8 Hz, 1H), 7.53 (d, J = 1.6 Hz, 1H), 7.31 (d, J = 8.8 Hz, 2H), 7.15- |
| | 7.18 (m, 1H), 7.07 (q, J = 8.4 Hz, 1H), 7.02 (t, J = 9.6 Hz, 1H), 4.95-5.02 (m, |
| | 2H), 3.42 (br, 1H), 3.28 (s, 1H), 2.80-2.90 (m, 2H), 2.25 (s, 3H), 1.65-1.72 (m, |
| | 2H), 1.30 (q, J = 8.0 Hz, 2H), 0.90-0.96 (m, 3H). |
| 285 | MS: m/z 528.9 [M + H]⁺; ¹H NMR (400 MHz, DMSO-d₆) δ 11.19 (s, 1H), 8.81 (d, |
| | J = 4.8 Hz, 1H), 8.16 (s, 1H), 7.63 (s, 1H), 7.57 (d, J = 2.0 Hz, 1H), 7.44 (t, J = 7.0 |
| | Hz, 1H), 7.34 (d, J = 8.6 Hz, 2H), 7.19 (s, 1H), 7.17 (d, J = 4.9 Hz, 1H), 7.10 (dd, J = |
| | 8.6, 2.1 Hz, 1H), 7.04 (t, J = 7.2 Hz, 1H), 4.94 (s, 1H), 3.46 (s, 2H), 2.96-2.86 (m, |
| | 2H). |
| 302 | MS: m/z 512.0 [M + H]⁺; ¹H NMR (DMSO-d₆) δ: 10.71 (s, 1H), 8.80 (br d, J = 4.0 |
| | Hz, 1H), 8.35 (s, 2H), 7.21 (d, J = 8.9 Hz, 1H), 7.09-7.15 (m, 1H), 6.95-7.09 (m, |
| | 1H), 6.75 (br d, J = 8.5 Hz, 1H), 6.66-6.88 (m, 1H), 4.86-5.19 (m, 1H), 3.79 (s, 3H), |
| | 3.66 (br dd, J = 18.6, 4.3 Hz, 8H), 3.35-3.48 (m, 1H), 2.88-3.01 (m, 1H), 2.72-2.88 |
| | (m, 1H) |
| 303 | MS: m/z 515.0 [M + H]⁺; ¹H NMR (400 MHz, DMSO) δ 11.14(s, 1H), 8.77(d, J = |
| | 4.4 Hz, 1H), 8.13(d, J = 2.4 Hz, 1H), 7.54 (d, J = 2.0 Hz, 1H), 7.44 (dd, J = 9, 2.4 |
| | Hz, 1H), 7.31 (d, J = 8.4 Hz, 1H), 7.10-7.06 (m, 2H), 6.94 (s, 1H), 6.79 (d, J = 8.0 |
| | Hz, 1H), 4.96 (s, 1H), 3.67-3.64 (m, 4 H), 3.41-3.38 (m, 4 H), 3.30-3.29 (m, 1H), |
| | 2.94-2.77 (m, 2H) |
| 305 | MS: m/z 488.0 [M − H]⁻; ¹H NMR (DMSO-d₆, 400 MHz), δ 11.75 (s, 1H), 8.80- |
| | 8.79 (d, J = 4.8 Hz, 1H), 8.52-8.52 (d, J = 2.0 Hz, 1H), 8.02-7.99 (dd, J = 2.4 Hz, |
| | 8.8 Hz, 1H), 7.49-7.47 (d, J = 8.8 Hz, 1H), 7.30 (brs, 1H), 7.16-7.14 (d, J = |
| | 5.2 Hz, 1H), 7.06-7.02 (t, J = 7.2 Hz, 1H), 6.83-6.80 (t, J = 7.2 Hz, 1H), 4.96-4.93 |
| | (d, J = 11.2 Hz, 1H), 3.46-3.40 (d, J = 12.0 Hz, 1H), 3.07-3.02 (dd, J = 2.8 Hz, 16.0 |
| | Hz, 1H), 2.95-2.88 (m, 1H), 2.26-2.26 (d, J = 1.2 Hz, 3H). |
| 319 | MS: m/z 498.0 [M + H]⁺; ¹H NMR (400 MHz, CDCl₃) δ 8.59 (d, 1H, J = 4.8 Hz), |
| | 8.13 (br, 1H), 7.53 (d, 1H, J = 2.0 Hz), 7.46 (d, 1H, J = 8.0 Hz), 7.26 (d, 1H, J = |
| | 8.0 Hz), 7.24 (s, 1H), 7.20-7.17 (m, 2H), 6.91 (d, 1H, 4.8 Hz), 5.13-5.07 (m, |
| | 1H), 3.63-3.56 (m, 1H), 3.07-2.90 (m, 2H) |
| 323 | MS: m/z 524.9 [M − H]⁻; ¹H NMR (CDCl₃, 400 MHz) 8.51-8.50 (d, J = 4.8 Hz, 1H), |
| | 8.46-8.45 (d, J = 2.4 Hz, 1H), 8.33 (brs, 1H), 8.31 (brs, 2H), 8.05-8.02 (dd, J = |
| | 2.0 Hz, 8.8 Hz, 1H), 7.25-7.23 (d, J = 8.8 Hz, 1H), 6.91 (s, 1H), 6.78-6.77 (d, J = |
| | 4.4 Hz, 1H), 5.16-5.11 (m, 1H), 3.70-3.68 (m, 4H), 3.65-3.63 (m, 4H), 3.30-3.25 |
| | (m, 1H), 2.95-2.92 (m, 2H). |
| 341 | MS: m/z 447.0 [M + H]⁺; ¹H NMR (400 MHz, DMSO) δ 11.20 (s, 1H), 8.79 |
| | (d, J = 4.4 Hz, 1H), 7.54 (s, 1H), 7.43-7.36 (m, 2H), 7.32 (d, J = 8.4 Hz, 1H), 7.25 (t, |
| | J = 9.6 Hz, 1H), 7.14 (q, J = 3.6 Hz, 2H), 7.08 (q, J = 6.8 Hz, 1H), 7.01 (t, J = 8.0 Hz, |
| | 1H), 4.86 (s, 1H), 3.32 (d, J = 5.2 Hz, 1H), 2.90-2.83 (m, 2H). |
| 343 | MS: m/z 443.0 [M + H]⁺; ¹H NMR (400 MHz, DMSO) δ 11.23 (s, 1H), 8.78 (s, |
| | 1H), 7.53 (d, J = 1.6 Hz, 1H), 7.31 (d, J = 8.8 Hz, 1H), 7.21 (d, J = 8.0 Hz, 2H), 7.16 |
| | (d, J = 8.0 Hz, 2H), 7.11-7.07 (m, 3H), 4.88 (s, 1H), 3.27 (t, J = 10.0 Hz, 1H), 2.90- |
| | 2.82 (m, 2H), 2.27 (s, 3H). |
| 349 | MS: m/z 460.8 [M − H]⁻; ¹H NMR (400 MHz, DMSO) δ 11.25(s, 1H), 8.80 (d, J = |
| | 4.4 Hz, 1H), 7.55 (d, J = 2 Hz, 1H), 7.44(t, J = 2 Hz, 2H), 7.33 (q, J = 6.4 Hz, 3H), |
| | 7.10 (m, 3H), 4.90(s, 1H), 3.23 (q, J = 8.4 Hz, 1H), 2.87(m, 2H). |
| 353 | MS: m/z 445.0 [M + H]⁺; ¹H NMR (DMSO-d, 400 MHz) 11.06(s, 1H), 9.78(s, 1H), |
| | 8.76(d, J = 4.8 Hz, 1H), 7.49(d, J = 2.0 Hz, 1H), 7.31 (d, J = 8.8 Hz, 1H), 7.29 (s, 1H), |
| | 7.17-7.12 (m, 1H), 7.09 (d, J = 4.8 Hz, 1H), 7.05 (q, J = 6.8 Hz, 1H), 6.87 (d, J = 8.0 |
| | Hz, 1H), 6.77-6.72 (m, 2H), 4.83 (d, J = 8.8 Hz, 1H), 3.42-3.37 (m, 1H), 2.86-2.76 |
| | (m, 2H |
| 354 | MS: m/z 460.9 [M + H]⁺; ¹H NMR (400 MHz, DMSO) δ 11.23(s, 1H), 8.78(d, J = |
| | 4.4 Hz, 1H), 7.54 (d, J = 2 Hz, 1H), 7.33(d, J = 8.8 Hz, 1H), 7.27(t, J = 8.4 Hz, 1H), |
| | 7.12(d, J = 5.2 Hz, 1H), 7.09(q, J = 2 Hz, 1H), 7.04(m, 3H), 4.92(d, J = 4 Hz, 1H), |
| | 3.27(m, 1H), 2.87(m, 2H), 2.20(d, J = 1.2 Hz, 3H). |
| 372 | MS: m/z 529.2 [M + H]⁺; ¹H NMR (400 MHz, DMSO) δ: 11.28 (s, 1H), 8.12 (d, |
| | J = 2.4 Hz, 1H), 7.53 (d, J = 2.0 Hz, 1H), 7.43 (dd, J = 3.4, 2.0 Hz, 1H), 7.30 (d, |
| | J = 8.4 Hz, 1H), 7.07 (dd, J = 8.8, 2.0 Hz, 1H), 7.03 (s, 1H), 6.91 (br, 1H), 6.79 (d, |
| | J = 6.4 Hz, 1H), 5.02-4.91 (m, 1H), 3.67-3.65 (m, 4H), 3.40-3.38 (m, 4H), 3.30- |
| | 3.26 (m, 1H), 2.93-2.75 (m, 2H), 2.47 (s, 3H). |
| 373 | MS: m/z 526.8 [M + H]⁺; ¹H NMR (400 MHz, DMSO) δ11.16(s, 1H), 8.77(d, |
| | J = 2.8 Hz, 1H), 8.06(q, J = 2.0 Hz, 1H), 7.54 (d, J = 2.0 Hz, 1H), 7.36-7.39 (m, 1H), |
| | 7.30 (d, J = 8.8 Hz, 1H), 7.06-7.10 (m, 2H), 6.92 (s, 1H), 6.59 (d, J = 8.8 Hz, 1H), |
| | 4.95 (s, 1H), 4.79 (s, 1H), 4.63 (s, 1H), 3.74 (t, J = 6.2 Hz, 1H), 3.58 (t, J = 7.0 Hz, |
| | 1H), 3.40 (d, J = 10.0 Hz, 1H), 3.32-3.35 (m, 1H), 3.17-3.19 (m, 1H), 2.90 (q, J = 3.6 |
| | Hz, 1H), 2.77-2.84 (m, 1H), 1.83 (q, J = 10.0 Hz, 2H) |
| 374 | MS: m/z 530.2 [M + H]⁺; ¹H NMR (400 MHz, DMSO) δ 11.08 (s, 1H), 8.33 (s, |
| | 2H), 7.55 (d, J = 2.0 Hz, 1H), 7.31 (d, J = 8.8 Hz, 1H), 7.08 (dd, J = 8.4, 2.0 Hz, |
| | 1H), 7.05 (s, 1H), 6.84 (br, 1H), 5.11-4.95 (m, 1H), 3.66-3.61 (m, 8H), 3.41-3.32 |
| | (m, 1H), 2.96-2.73 (m, 2H), 2.48 (s, 3H). |
| 422 | MS: m/z 447.1 [M + H]⁺; ¹H NMR (400 MHz, DMSO) δ 11.22 (s, 1H), 8.79 (d, J = |
| | 4.7 Hz, 1H), 7.54 (d, J = 1.9 Hz, 1H), 7.42-7.26 (m, 3H), 7.19 (t, J = 8.9 Hz, 2H), |
| | 7.14-7.05 (m, 3H), 4.89 (s, 1H), 3.30 (s, 1H), 2.87 (d, J = 4.3 Hz, 2H). |
| 423 | MS: m/z 465.1 [M + H]⁺; ¹H NMR (400 MHz, DMSO) δ 11.17 (s, 1H), 8.79 (d, J = |
| | 4.8 Hz, 1H), 7.54 (d, J = 2.0 Hz, 1H), 7.37-7.20 (m, 3H), 7.20-6.94 (m, 4H), 4.87 |
| | (d, J = 10.9 Hz, 1H), 3.46-3.13 (m, 1H), 2.85 (dd, J = 12.7, 4.0 Hz, 2H). |
| 428 | MS: m/z 601.2 [M + H]⁺; ¹H NMR (400 MHz, DMSO) δ 11.10 (d, J = 33.2 Hz, 1H), |
| | 8.09 (dd, J = 2.4, 2.4 Hz, 1H), 7.53-7.55 (m, 1H), 7.39-7.44 (m, 1H), 7.29-7.31 (m, |
| | 1H), 7.07-7.10 (m, 1H), 6.95 (d, J = 2.4 Hz, 1H), 6.80 (d, J = 9.2 Hz, 1H), 4.89-4.95 |
| | (m, 1H), 3.90-4.05 (m, 1H), 3.64-3.84 (m, 12H), 3.18-3.35 (m, 4H), 2.75-2.94 (m, |
| | 2H). |
| 429 | MS: m/z 516.1 [M + H]⁺; ¹H NMR (400 MHz, DMSO) δ 11.16 (s, 1H), 9.22 (s, |
| | 1H), 8.15 (s, 1H), 7.56 (d, J = 2.0 Hz, 1H), 7.48 (d, J = 9.2 Hz, 1H), 7.32(d, J = 8.8 |
| | Hz, 1H), 7.09 (dd, J = 8.6, 2.0 Hz, 1H), 6.81 (d, J = 9.2 Hz, 1H), 5.24-4.77(m, 1H), |
| | 3.66 (t, J = 4.8 Hz, 4H), 3.51-3.48 (m, 1H), 3.41 (t, J = 4.8 Hz, 4H), 3.01-2.89 (m, |
| | 2H). |
| 430 | MS: m/z 443.9 [M + H]⁺; ¹H NMR (400 MHz, DMSO) δ 11.24 (s, 1H), 9.22 (s, |
| | 1H), 7.56 (d, J = 2.0 Hz, 1H), 7.33 (d, J = 8.8 Hz, 1H), 7.24 (d, J = 6.8 Hz, 2H), |
| | 7.18 (d, J = 8.4 Hz, 2H), 7.09 (dd, J = 8.4, 2.0 Hz, 1H), 5.20-4.76(m, 1H), 3.51- |
| | 3.40 (m, 2H), 2.98-2.88 (m, 2H), 2.27 (s, 3H). |
| 431 | MS: m/z 443.9 [M + H]⁺; ¹H NMR (400 MHz, DMSO) δ 11.16 (s, 1H), 8.91 (s, |
| | 1H), 7.57 (d, J = 2.5 Hz, 1H), 7.34 (d, J = 8.4 Hz, 1H), 7.10(dd, J = 8.4, 2.0 Hz, |
| | 1H), 7.06 (d, J = 7.2 Hz, 1H), 6.81 (t, J = 7.0 Hz, 1H), 5.09-4.98(m, 1H), 3.54-3.48 |
| | (m, 2H), 3.00-2.91 (m, 2H), 2.27 (d, J = 1.2 Hz, 3H). |
| 437 | MS: m/z 458.3 [M + H]⁺; ¹H NMR (400 MHz, DMSO-d₆) δ 11.26 (d, 1H, |
| | J = 13.2 Hz), 7.55 (t, 1H, J = 2.2 Hz), 7.35-7.31 (m, 1H), 7.23-7.17 (m, 4.4 H), 7.09 |
| | (dd, 1H, J = 8.4 Hz, 2.0 Hz), 7.03 (s, 0.6 H), 5.01-4.82 (m, 1H), 3.31-3.24 (m, 1H), |
| | 2.97-2.79 (m, 2H), 2.52 (d, 3 H, J = 28.4 Hz), 2.28 (d, 3 H, J = 2.8 Hz) |
| 438 | MS: m/z 493.9 [M + H]⁺; ¹H NMR (400 MHz, CDCl3) δ 7.99 (d, 1H, J = 11.6 Hz), |
| | 7.52 (d, 1H, J = 2.0 Hz), 7.37 (s, 0.5 H), 7.26-7.24 (m, 1.5 H), 7.18-7.16 (m, 1H), |
| | 6.91-6.79 (m, 2H), 5.32-5.22 (m, 1H), 3.55-3.47 (m, 1H), 2.99-2.91 (m, 2H), 2.57 |
| | (d, 3 H, J = 8.8 Hz), 2.32-2.30 (m, 3 H) |
| 439 | MS: m/z 530.2 [M + H]⁺; ¹H NMR (400 MHz, DMSO-d₆) δ 11.16 (d, 1H, J = 12.8 |
| | Hz), 8.13 (dd, 1H, J = 13.6 Hz, 2.4 Hz), 7.56 (br, 1H), 7.47-7.41 (m, 1H), 7.32 (dd, |
| | 1H, J = 8.8 Hz, 3.6 Hz), 7.09 (dd, 1H, J = 8.8 Hz, 2.0 Hz), 6.99 (d, 1H, J = 60.4 Hz), |
| | 6.82 (d, 1H, J = 8.8 Hz), 5.06-4.87 (m, 1H), 3.66 (s, 4 H), 3.42-3.40 (m, 5 H), 2.99- |
| | 2.95 (m, 1H), 2.85-2.78 (m, 1H), 2.52 (d, 3 H, J = 30.0 Hz) |
| 440 | MS: m/z 531.2 [M + H]⁺; ¹H NMR (400 MHz, DMSO-d₆) δ 11.12 (d, 1H, J = 16.8 |
| | Hz), 8.34 (d, 2H, J = 15.2 Hz), 7.57 (br, 1H), 7.33 (dd, 1H, J = 8.4 Hz, 2.0 Hz), 7.10 |
| | (dd, 1H, J = 8.4 Hz, 2.0 Hz), 6.87 (d, 1H, J = 60.4 Hz), 5.10-4.91 (m, 1H), 3.67 (d, 4 |
| | H, J = 2.4 Hz), 3.62 (d, 4 H, J = 2.4 Hz), 3.48-3.40 (m, 1H), 3.02-2.94 (m, 1H), 2.85- |
| | 2.76 (m, 1H), 2.53 (d, 3 H, J = 28.0 Hz) |
| 447 | MS: m/z 454.1 [M + H]⁺; ¹H NMR (400 MHz, DMSO) δ 11.25 (s, 1H), 8.80 (d, J = |
| | 4.8 Hz, 1H), 7.85 (d, J = 8.3 Hz, 2H), 7.54 (dd, J = 15.5, 5.1 Hz, 3H), 7.34 (t, J = |
| | 10.9 Hz, 1H), 7.19-7.04 (m, 3H), 4.91 (s, 1H), 3.30-3.13 (m, 1H), 2.98-2.75 (m, |
| | 2H). |
| 458 | MS: m/z 516.9 [M + H]⁺; ¹H NMR (400 MHz, DMSO) δ 11.15 (s, 1H), 8.88 (s, |
| | 1H), 8.38 (s, 2H), 7.57 (d, J = 2.0 Hz, 1H), 7.34 (d, J = 8.4 Hz, 1H), 7.10 (dd, |
| | J = 8.6, 2.0 Hz, 1H), 5.11 (br, 1H), 3.69-3.61 (m, 8H), 3.58-3.50 (m, 1H), 3.03-2.98 |
| | (m, 1H), 2.91-2.83 (m, 1H) |
| 469 | MS: m/z 487.0 [M + H]⁺; (DMSO-d, 400 MHz) 11.13 (s, 1H), 8.53 (d, J = 5.2 Hz, |
| | 1H), 8.20-8.17 (m, 2H), 7.57-7.52 (m, 5H), 7.31 (d, J = 4.0 Hz, 1H), 7.30 (s, 1H), |
| | 7.07 (q, J = 6.4 Hz, 1H), 7.02 (d, J = 7.6 Hz, 1H), 6.76 (t, J = 6.8 Hz, 1H), 5.01 (d, |
| | J = 11.6 Hz, 1H), 3.38 (t, J = 7.2 Hz, 1H), 2.86 (t, J = 4.8 Hz, 2H), 2.26 (d, J = 1.2 Hz, |
| | 3H). |
| 475 | MS: m/z 524.0 [M + H]⁺; (CDCl3, 400 MHz), : 8.35-8.33 (d, J = 5.2 Hz 1H), 8.32 |
| | (s, 2H), 8.01-7.98 (m, 2H), 7.91 (s, 1H), 7.44-7.41 (m, 4 H), 7.11-7.03 (m, 3 H), |
| | 6.91-6.90 (d, J = 5.2 Hz 1H), 5.17-5.14 (d, J = 13.2 Hz, 1H), 3.74-3.66 (m, 4 H), |
| | 3.64-3.63 (m, 4 H), 3.26-3.18 (m, 1H), 2.95-2.87 (m, 1H), 2.80-2.76 (dd, J = |
| | 3.2 Hz, 15.2 Hz, 1H). |

### Example 15

### Preparation of Compound 340

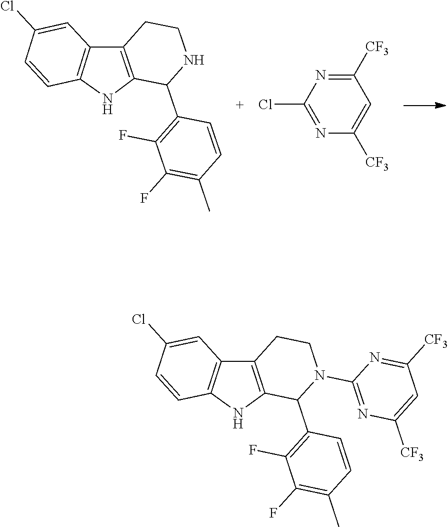 [SMILES: Cc1ccc(C2NCCc3c2[nH]c2ccc(Cl)cc32)c(F)c1F ; FC(F)(F)c1cc(C(F)(F)F)nc(Cl)n1 ; Cc1ccc(C2c3[nH]c4ccc(Cl)cc4c3CCN2c2nc(C(F)(F)F)cc(C(F)(F)F)n2)c(F)c1F]

A 50 mL flask equipped with a stir bar was charged with 6-chloro-1-(2,3-difluoro-4-methyl-phenyl)-2,3,4,9-tetrahydro-1H-pyrido[3,4-b]indole (200 mg, 0.601 mmol) and tetrahydrofuran (10 mL). 2-chloro-4,6-(bis-trifluoromethyl)pyrimidine (181 mg, 0.722 mmol) was added by syringe under stirring at 22° C. followed by N,N-diisopropylamine (0.21 mL, 1.200 mmol). The flask was allowed to stir at 90° C. for 5 h. After cooling to ambient temperature the volatiles were removed under reduced pressure and the obtained dark oily residue subjected to purification by silica-gel column chromatography eluting with a gradient (0-15%) dichloromethane/methanol. Compound 340 (41 mg, 0.075 mmol, 12.5% yield) was isolated as a yellow amorphous solid. MS m/z 544.9 [M−H]⁻; ¹H NMR (400 MHz, DMSO-d₆) δ: 11.16 (s, 1H), 7.56 (s, 2H), 7.32-7.30 (d, J=8.4 Hz, 1H), 7.18 (s, 1H), 7.10-7.07 (dd, J=2.0, 8.4 Hz, 1H), 7.05-7.01 (t, J=7.2 Hz, 1H), 6.83-6.80 (t, J=7.2 Hz, 1H), 4.91-4.87 (dd, J=4.8, 13.6 Hz, 1H), 3.55-3.47 (m, 1H), 3.00-2.95 (dd, J=4.8, 16.0 Hz, 1H), 2.91-2.85 (m, 1H), 2.25-2.25 (d, J=1.2 Hz, 3H).

Using the procedure described for Compound 340 above, additional compounds described herein were prepared by substituting the appropriate starting materials, suitable reagents and reaction conditions, obtaining compounds such as those selected from:

| Cpd | Data |
|---|---|
| 91 | MS m/z 413.0. [M + H]⁺; ¹H NMR (400 MHz, DMSO) δ 11.27 (s, 1H), 8.63 (s, 2H), |
| | 8.56 (s, 2H), 7.69 (d, J = 8.0 Hz, 1H), 7.36 (d, J = 8.0 Hz, 1H), 6.91 (s, 1H), 4.89 |
| | (dd, J = 12.0, 4.0 Hz, 1H), 3.32-3.28 (m, 1H), 2.90-2.76 (m, 2H), 2.59 (s, 3H) |
| 92 | MS m/z 463.0. [M + H]⁺; ¹H NMR (400 MHz, DMSO) δ 11.24 (s, 1H), 8.80 (d, J = 4.0 |
| | Hz, 1H), 8.66 (s, 2H), 7.72 (d, J = 8.0 Hz, 1H), 7.37 (d, J = 8.0 Hz, 1H), 7.15 (d, J = 4.0 |
| | Hz, 1H), 6.92 (s, 1H), 5.04-4.93(m, 1H), 3.45-3.39(m, 1H), 3.98-2.82(m, 2H), 2.60(s, 3H) |
| 94 | MS m/z 438.0. [M + H]⁺; ¹H NMR (400 MHz, DMSO-d₆) δ 11.18 (s, 1H), 8.29 (d, |
| | J = 5.2 Hz, 1H), 8.27 (s, 2H), 7.66 (d, J = 7.2 Hz, 1H), 7.31 (d, J = 10.4 Hz, 1H), 6.88 |
| | (s, 1H), 6.58 (d, J = 5.2 Hz, 1H), 5.02-4.97 (m, 1H), 3.26-3.19 (m, 1H), 3.07 (s, 6H), |
| | 2.88-2.74 (m, 2H), 2.34 (s, 3H). |
| 95 | MS m/z 492.0 [M + H]⁺; ¹H NMR (400 MHz, DMSO-d₆) δ 11.19 (s, 1H), 8.78 (d, J = 4.8 |
| | Hz, 1H), 8.29 (s, 2H), 7.69 (d, J = 7.6 Hz, 1H), 7.33 (d, J = 10.0 Hz, 1H), 7.11 (d, |
| | J = 4.8 Hz, 1H), 6.84-6.73 (m, 1H), 5.00-4.98 (m, 1H), 3.42-3.38 (m, 1H), 3.08 (s, |
| | 6H), 2.96-2.75 (m, 2H). |
| 96 | MS m/z 475.0 [M + H]⁺; ¹H NMR (400 MHz, DMSO) δ(ppm), 11.28(s, 1H), 7.66(d, |
| | J = 7.2 Hz, 1H), 7.31(d, J = 10.0 Hz, 1H), 7.20-6.96(m, 6H), 5.04-4.74(m, 1H), |
| | 3.25-3.19(m, 1H), 2.88-2.76(m, 2H), 2.46-2.38(m, 3H), 2.26(s, 3H). |
| 204 | MS m/z 491.0 [M − H]⁻; ¹H NMR (DMSO, 400 MHz) δ: 11.15 (s, 1H), 7.54 (d, 2.0 Hz, |
| | 1H), 7.32-7.21 (m, 2H), 7.10-7.06 (m, 2H), 7.02 (t, J = 7.2 Hz, 1H), 6.77 (t, J = 7.2 Hz, |
| | 1H), 5.01-4.82 (m, 1H), 3.42-3.34 (m, 1H), 2.92-2.81 (m, 2H), 2.46 (s, 3H), 2.25 (s, 3H). |
| 286 | MS m/z 403.0 [M + H]⁺; ¹H NMR (400 MHz, DMSO-d₆) δ 11.18 (s, 1H), 7.49 (d, J = 2.0 |
| | Hz, 1H), 7.30 (d, J = 8.4 Hz, 1H), 7.19-7.17 (m, 3H), 7.14-7.12 (m, 2H), 7.06 (dd, J = 8.8, |
| | 2.4 Hz, 1H), 6.45 (s, 1H), 4.93-4.89 (m, 1H), 3.12-3.07 (m, 1H), 2.77-2.75 |
| | (m, 2H), 2.27-2.26 (m, 9H). |
| 289 | MS m/z = 497.0 [M + H]⁺; ¹H NMR (500 MHz, DMSO-d₆) δ 11.09 (d, J = 16.6 Hz, 1H), |
| | 8.32 (d, J = 10.7 Hz, 2H), 7.55 (d, J = 2.1 Hz, 1H), 7.32 (dd, J = 8.6, 2.6 Hz, 1H), 7.09 (dd, |
| | J = 8.5, 2.1 Hz, 1H), 6.81 (d, J = 83.0 Hz, 1H), 4.92 (ddd, J = 85.3, 13.2, 5.3 Hz, 1H), |
| | 3.78-3.55 (m, 8H), 3.46-3.33 (m, 1H), 2.95 (dt, J = 14.8, 4.1 Hz, 1H), 2.86-2.70 (m, 1H), |
| | 2.41 (d, J = 33.9 Hz, 3H). |
| 306 | MS m/z 454.0 [M + H]⁺; ¹H NMR (400 MHz, CD₃OD) δ 11.03 (s, 1H), 7.89 (d, J = 6.0 Hz, |
| | 1H), 7.48 (d, J = 2.0 Hz, 1H), 7.37 (s, 1H), 7.28 (d, J = 8.8 Hz, 1H), 7.05 (dd, J = 2.2 Hz, |
| | 1H), 7.00 (t, J = 7.4 Hz, 1H), 6.72 (t, J = 7.2 Hz, 1H), 6.00 (d, J = 6.0 Hz, 1H), 4.85 (d, |
| | J = 16.4 Hz, 1H), 3.18-3.25 (m, 1H), 3.02 (s, 6H), 2.75-2.79 (m, 2H), 2.25 (d, J = 1.2 Hz, 3H). |
| 307 | MS m/z 418.0 [M + H]⁺; ¹H NMR (400 MHz, DMSO) δ 11.17 (s, 1H), 7.91 (d, J = 6 Hz, |
| | 1H), 7.49 (d, J = 2 Hz, 1H), 7.30 (d, J = 8.4 Hz, 1H), 7.18 (d, J = 8 Hz, 2H), 7.13 (d, |
| | J = 7.6 Hz, 3H), 7.05 (q, J = 6.4 Hz, 1H), 5.98 (d, J = δ Hz, 1H), 4.84 (t, J = 4.4 Hz, 1H), |
| | 3.07 (d, J = 17.2 Hz, 7H), 2.76 (q, J = 9.2 Hz, 2H), 2.67 (s, 3H). |
| 308 | MS m/z 476.0 [M + H]⁺; ¹H NMR (400 MHz, DMSO) δ 11.06 (s, 1H), 8.32 (s, 2H), |
| | 7.52 (d, J = 2.0 Hz, 1H), 7.30 (d, J = 8.8 Hz, 1H), 7.07 (dd, J = 8.4, 2.0 Hz, 1H), |
| | 6.92 (s, 1H), 6.47 (s, 1H), 5.06-5.01 (m, 1H), 3.65-3.61 (m, 8H), 3.26-3.19 (m, |
| | 1H), 2.88-2.70 (m, 2H), 2.29 (s, 6H). |
| 312 | MS m/z 462.0 [M + H]⁺; ¹H NMR (400 MHz, DMSO) δ 11.08 (s, 1H), 8.32 (s, 2H), |
| | 8.30 (d, J = 4.8 Hz, 1H), 7.52 (d, J = 2.0 Hz, 1H), 7.31 (d, J = 8.8 Hz, 1H), 7.07 (dd, |
| | J = 8.4, 2.0 Hz, 1H), 6.90 (s, 1H), 6.59 (d, J = 4.8 Hz, 1H), 5.03-4.97 (m, 1H), |
| | 3.66-3.61 (m, 8H), 3.29-3.22 (m, 1H), 2.88-2.68 (m, 2H), 2.34 (s, 3H). |
| 313 | MS m/z 486.0 [M − H]⁻; ¹H NMR (DMSO-d6, 400 MHz) δ 11.68 (s, 1H), 8.52-8.51 (d, |
| | J = 2.4 Hz, 1H), 8.36 (s, 2H), 8.02-7.99 (dd, J = 2.4 Hz, 8.8 Hz, 1H), 7.49-7.47 (d, J = 8.8 Hz, |
| | 1H), 6.94 (s, 1H), 5.09-5.04 (dd, J = 5.2 Hz, 13.6 Hz, 1H), 3.68-3.66 (m, 4H), |
| | 3.62-3.60 (m, 4H), 3.30-3.28 (m, 1H), 3.08-3.03 (dd, J = 2.8 Hz, 15.6 Hz, 1H), 2.87-2.83 |
| | (m, 1H), 2.40 (s, 3H), 2.34 (s, 3H). |
| 324 | MS m/z 476.0 [M + H]⁺; ¹H NMR (400 MHz, DMSO) δ 11.14 (s, 1H), 8.10 (d, J = 2.0 |
| | Hz, 1H), 7.53 (d, J = 1.2 Hz, 1H), 7.42 (dd, J = 8.8, 2.4 Hz, 1H), 7.31 (d, J = 8.8 |
| | Hz, 1H), 7.08 (dd, J = 8.8, 2.0 Hz, 1H), 7.03 (s, 1H), 6.79 (d, J = 8.0 Hz, 1H), |
| | 5.00-4.96 (m, 1H), 3.67-3.65 (m, 4H), 3.41-3.38 (m, 4H), 3.24-3.16 (s, 1H), |
| | 2.92-2.68 (m, 2H), 2.40 (s, 3H), 2.33 (s, 3H). |
| 332 | MS m/z 389.0 [M + H]⁺; ¹H NMR (400 MHz, DMSO) δ 11.21 (s, 1H), 8.28 (d, J = 5.2 |
| | Hz, 1H), 7.49 (d, J = 1.6 Hz, 1H), 7.30 (d, J = 8.8 Hz, 1H), 7.19-7.17 (m, 2H), 7.15-7.13 |
| | (m, 3H), 7.06 (dd, J = 8.8, 2.0 Hz, 1H), 6.56 (d, J = 4.8 Hz, 1H), 4.90-4.87 (m, 1H), |
| | 3.14-3.09 (m, 1H), 2.78-2.76 (m, 2H), 2.33 (s, 3H), 2.26 (s, 3H). |
| 334 | MS m/z = 498.0 [M + H]⁺; ¹H NMR (DMSO-d₆) δ: 11.10 (s, 1H), 8.67 (br d, J = 4.8 |
| | Hz, 1H), 8.35 (s, 2H), 7.54 (s, 1H), 7.32 (d, J = 8.5 Hz, 1H), 7.08 (br d, J = 8.5 Hz, |
| | 1H), 6.93 (d, J = 4.8 Hz, 1H), 6.86 (m, 1H), 4.87-5.13 (m, 1H), 3.77-3.96 (m, 1H), |
| | 3.55-3.73 (m, 8H), 3.27-3.42 (m, 1H), 2.87-3.02 (m, 1H), 2.70-2.84 (m, 1H) |
| 335 | MS m/z = 528.0 [M + H]⁺; ¹H NMR (500 MHz, DMSO-d₆) δ 11.09 (s, 1H), 8.78 (d, |
| | J = 4.7 Hz, 1H), 8.30 (s, 2H), 7.68-7.45 (m, 1H), 7.42-7.23 (m, 1H), 7.19-6.98 |
| | (m, 2H), 6.80 (s, 1H), 5.24-4.54 (m, 3H), 3.68 (dt, J = 79.9, 7.3 Hz, 2H), 3.51-3.14 |
| | (m, 39H), 2.88 (dd, J = 62.1, 14.9 Hz, 2H), 1.84 (q, J = 10.4 Hz, 2H). |
| 336 | MS m/z = 489.0 [M + H]⁺; ¹H NMR (500 MHz, DMSO-d₆) δ 11.30-10.83 (m, 1H), |
| | 8.29 (s, 2H), 7.81 (d, J = 87.3 Hz, 1H), 7.54 (s, 1H), 7.44-7.20 (m, 1H), 7.07 (t, J = 10.3 |
| | Hz, 1H), 6.91 (s, 1H), 5.03 (d, J = 13.4 Hz, 1H), 4.88 (s, 1H), 4.64 (s, 1H), 3.99-3.19 |
| | (m, 8H), 3.08-2.65 (m, 3H), 2.46-2.15 (m, 5H), 1.85 (d, J = 9.0 Hz, 1H). |
| 337 | MS m/z = 542.0 [M + H]⁺; ¹H NMR (500 MHz, DMSO-d₆) δ 11.06 (d, J = 43.6 Hz, |
| | 1H), 8.79 (d, J = 4.9 Hz, 1H), 8.43-8.20 (m, 2H), 7.68-7.45 (m, 1H), 7.39-7.21 |
| | (m, 1H), 7.20-6.70 (m, 3H), 5.00 (s, 1H), 4.56 (s, 2H), 3.75-3.19 (m, 6H), |
| | 3.09-2.68 (m, 2H), 2.13-1.65 (m, 4H). |
| 338 | MS m/z = 503.0 [M + H]⁺; ¹H NMR (500 MHz, DMSO-d₆) δ 11.06 (d, J = 52.8 Hz, |
| | 1H), 8.32 (s, 2H), 7.99-7.43 (m, 1H), 7.41-7.19 (m, 1H), 7.17-6.79 (m, 2H), |
| | 5.03 (dd, J = 13.3, 5.4 Hz, 1H), 4.56 (s, 1H), 3.55 (d, J = 5.5 Hz, 3H), 3.13-2.64 |
| | (m, 2H), 2.45-2.09 (m, 5H), 2.05-1.74 (m, 3H). |
| 344 | MS m/z = 486.0 [M + H]⁺; ¹H NMR (400 MHz, DMSO-d₆) δ 11.08 (s, 1H), 8.78 (d, |
| | J = 4.8 Hz, 1H), 8.28 (s, 2H), 7.54 (d, J = 2.0 Hz, 1H), 7.31 (d, J = 8.6 Hz, 1H), |
| | 7.19-7.00 (m, 2H), 6.79 (s, 1H), 4.98 (s, 1H), 3.99 (t, J = 7.5 Hz, 4H), 3.54-3.33 (m, |
| | 1H), 2.93 (dd, J = 15.4, 3.9 Hz, 1H), 2.88-2.72 (m, 1H), 2.27 (p, J = 7.5 Hz, 2H). |
| 345 | MS m/z = 447.0 [M + H]⁺; ¹H NMR (400 MHz, DMSO-d₆) δ 10.98 (d, J = 32.8 Hz, 1H), |
| | 8.21 (s, 2H), 7.89-7.42 (m, 1H), 7.32-7.13 (m, 1H), 7.00 (td, J = 8.7, 1.9 Hz, 1H), 6.83 (s, |
| | 1H), 4.95 (dd, J = 13.6, 5.3 Hz, 1H), 3.93 (t, J = 7.5 Hz, 3H), 3.18 (dt, J = 12.7, 6.6 Hz, |
| | 1H), 2.84 (dd, J = 16.2, 4.0 Hz, 1H), 2.68 (td, J = 16.1, 13.8, 5.2 Hz, 1H), 2.38-2.13 (m, 8H). |
| 346 | MS m/z = 438.0 [M + H]⁺; ¹H NMR (400 MHz, DMSO-d₆) δ 11.12 (s, 1H), 8.60 (s, |
| | 2H), 7.57 (d, J = 2.0 Hz, 1H), 7.35 (d, J = 8.6 Hz, 1H), 7.11 (dd, J = 8.5, 2.1 Hz, 1H), |
| | 6.99 (s, 1H), 5.06 (dd, J = 13.5, 5.2 Hz, 1H), 3.55-3.17 (m, 2H), 2.94 (dd, J = 15.4, |
| | 3.8 Hz, 1H), 2.78 (ddd, J = 16.0, 11.3, 5.4 Hz, 1H), 2.38 (d, J = 24.7 Hz, 6H). |
| 350 | MS m/z 475.0 [M + H]⁺; ¹H NMR (400 MHz, DMSO) δ 11.11 (s, 1H), 8.10 (d, J = 2.4 Hz, |
| | 1H), 7.50 (d, J = 2.0 Hz, 1H), 7.42 (dd, J = 8.8, 2.4 Hz, 1H), 7.29 (d, J = 8.4 Hz, 1H), |
| | 7.07-7.05 (m, 2H), 6.78 (d, J = 8.8 Hz, 1H), 6.45 (s, 1H), 4.99-4.95 (m, 1H), 3.67-3.64 (m, |
| | 4H), 3.39-3.37 (m, 4H), 3.19-3.12 (m, 1H), 2.84-2.75 (m, 2H), 2.28 (s, 6H). |
| 352 | MS m/z 461.0 [M + H]⁺; ¹H NMR (400 MHz, DMSO) δ 11.13 (s, 1H), 8.29 (d, J = 4.8 |
| | Hz, 1H), 8.19 (d, J = 2.4 Hz, 1H), 7.50 (d, J = 2.0 Hz, 1H), 7.43 (dd, J = 6.6, 2.4 |
| | Hz, 1H), 7.30 (d, J = 8.4 Hz, 1H), 7.06 (dd, J = 8.4, 2.0 Hz, 1H), 7.03 (s, 1H), 6.79 |
| | (d, J = 8.8 Hz, 1H), 6.57 (d, J = 4.8 Hz, 1H), 4.97-4.93 (m, 1H), 3.67-3.65 (m, |
| | 4H), 3.40-3.35 (m, 4H), 3.22-3.14 (m, 1H), 2.85-2.73 (m, 2H), 2.34 (s, 3H). |
| 355 | MS m/z 404.0 [M + H]⁺; ¹H NMR (400 MHz, DMSO) δ 11.23 (s, 1H), 7.52 (d, J = 2.0 Hz, |
| | 1H), 7.32 (d, J = 8.4 Hz, 1H), 7.19-7.14 (m, 5H), 7.08 (dd, J = 8.6, 2.0 Hz, 1H), 4.95-4.90 |
| | (m, 1H), 3.16-3.10 (m, 1H), 2.88-2.73 (m, 2H), 2.39 (s, 3H), 2.33 (s, 3H), 2.27 (s, 3H). |
| 356 | MS m/z 472.0 [M + H]⁺; ¹H NMR (400 MHz, DMSO) δ 11.14 (s, 1H), 8.75 (d, J = |
| | 4.4 Hz, 1H), 8.10 (d, J = 2.0 Hz, 1H), 7.53 (d, J = 2.0 Hz, 1H), 7.43 (dd, J = 8.8, 2.4 |
| | Hz, 1H), 7.31 (d, J = 8.4 Hz, 1H), 7.24 (d, J = 4.4 Hz, 1H), 7.08 (dd, J = 8.6, 2.0 |
| | Hz, 1H), 6.91 (br, 1H), 6.80 (d, J = 8.0 Hz, 1H), 4.88 (br, 1H), 6.67-3.65 (m, 4H), |
| | 3.41-3.39 (m, 4H), 3.31-3.26 (m, 1H), 2.92-2.76 (m, 2H). |
| 359 | MS m/z 472.9 [M + H]⁺; ¹H NMR (400 MHz, DMSO-d₆) δ 11.09 (s, 1H), 8.75 (d, J = 4.8 |
| | Hz, 1H), 8.33 (s, 2H), 7.54 (d, J = 1.6 Hz, 1H), 7.32 (d, J = 8.4 Hz, 1H), 7.24 d, J = 4.4 Hz, |
| | (1H), 7.08 (q, J = 2.2 Hz, 1H), 6.79 (s, 1H), 4.93 (d, J = 8.8 Hz, 1H), 3.66 (d, J = 4.8 Hz, |
| | 4H), 3.61 (d, J = 4.0 Hz, 4H), 3.34-3.38 (m, 1H), 2.92 (d, J = 2.8 Hz, 1H), 2.76-2.83 (m, 1H). |
| 361 | MS m/z 455.0 [M − H]⁻; (CDCl₃, 400 MHz) δ 7.74 (s, 1H), 7.52-7.51 (d, J = 2.0 Hz, |
| | 1H), 7.32-7.30 (d, J = 8.0 Hz, 2H), 7.19-7.10 (m, 5 H), 6.67 (s, 1H), 5.14-5.10 |
| | (dd, J = 2.0 Hz, 12.0 Hz, 1H), 3.40-3.32 (dd, J = 4.0 Hz, 12.8 Hz, 1H), 2.99-2.91 |
| | (m, 1H), 2.87-2.82 (dd, J = 3.6 Hz, 15.2 Hz, 1H), 2.45 (s, 3H), 2.31 (s, 3H). |
| 379 | MS m/z 426.2 [M + H]⁺; S m/z 414.1 [M + H]+; ¹H NMR (400 MHz, DMSO-d₆) δ |
| | 11.20 (s, 1H), 7.52 (d, J = 1.6 Hz, 1H), 7.31 (d, J = 8.4 Hz, 1H), 7.15-7.19 (m, 5H), |
| | 6.97-7.09 (m, 2H), 4.83 (d, J = 62.4 Hz, 1H), 3.16-3.25 (m, 1H), 2.75-2.87 (m, 2H), |
| | 2.43 (s, 3H), 2.27 (s, 3H). |
| 391 | MS m/z = 528.0 [M + H]⁺; ¹H NMR (400 MHz, DMSO-d₆) δ 11.07 (s, 1H), 8.78 (d, |
| | J = 4.8 Hz, 1H), 8.30 (s, 2H), 7.54 (d, J = 2.1 Hz, 1H), 7.31 (d, J = 8.6 Hz, 1H), 7.19-6.98 |
| | (m, 2H), 6.79 (s, 1H), 4.68 (s, 4H), 4.16 (s, 4H), 3.53-3.21 (m, 2H), 3.11-2.69 (m, 2H). |
| 407 | MS m/z 547.1 [M + H]⁺; ¹H NMR (DMSO-d₆, 400 MHz) δ: 11.16 (s, 1H), 7.5-7.6 |
| | (m, 2H), 7.32 (d, J = 8.5 Hz, 1H), 7.19 (s, 1H), 7.0-7.1 (m, 2H), 6.82 (t, J = 7.3 Hz, |
| | 1H), 4.8-5.0 (m, 1H), 3.4-3.6 (m, 1H), 2.8-3.1 (m, 2H), 2.26 (s, 3H). |
| 432 | MS m/z 533.0 [M + H]⁺; δ11.13(s, 1H), 8.77-8.76(d, J = 4.8 Hz, 1H), 7.52(d, J = 2.0 |
| | Hz, 1H), 7.33-7.28 (m, 2H), 7.11-7.05(m, 3H), 6.63-6.60)(m, 1H), 4.91(s, 1H), |
| | 3.66-3.64(m, 4 H), 3.41-3.36(m, 4 H), 2.89-2.79(m, 2H). |
| 448 | MS m/z 533.0 [M + H]⁺; ¹H NMR (400 MHz, DMSO) δ 11.27 (s, 1H), 7.84 (d, |
| | J = 8.4 Hz, 2H), 7.54 (d, J = 2.0 Hz, 1H), 7.49 (d, J = 8.2 Hz, 2H), 7.35 (d, J = 8.6 |
| | Hz, 1H), 7.21 (s, 1H), 7.10 (dd, J = 8.6, 2.1 Hz, 1H), 4.96 (dd, J = 13.3, 4.8 Hz, 1H), |
| | 3.23-3.03 (m, 1H), 2.84 (ddd, J = 16.6, 13.4, 4.1 Hz, 2H), 2.39 (s, 3H), 2.35 (s, 3H). |
| 449 | MS m/z 424.0 [M + H]⁺; ¹H NMR (400 MHz, DMSO) δ 11.23 (s, 1H), 7.42 (d, J = 8.5 Hz, |
| | 2H), 7.32 (d, J = 12.3, 8.6 Hz, 3H), 7.16 (s, 1H), 7.09 (dd, J = 8.6, 2.1 Hz, 1H), 4.94 (dd, |
| | J = 13.5, 5.1 Hz, 1H), 3.18-3.06 (m, 1H), 2.91-2.73 (m, 2H), 2.39 (s, 3H), 2.34 (s, 3H) |
| 450 | MS m/z 408.0 [M + H]⁺; ¹H NMR (400 MHz, DMSO) δ 11.24 (s, 1H), 7.55 (s, 1H), |
| | 7.34 (d, J = 8.3 Hz, 3H), 7.20 (t, J = 8.6 Hz, 3H), 7.10 (d, J = 8.6 Hz, 1H), 4.96 (d, |
| | J = 8.9 Hz, 1H), 3.67 (s, 1H), 3.34-2.72 (m, 2H), 2.46 (s, 3H), 2.39 (s, 3H). |
| 451 | ¹H NMR (400 MHz, DMSO-d₆) δ 11.15 (s, 1H), 7.53 (d, J = 1.9 Hz, 1H), 7.40-7.21 |
| | (m, 3H), 7.15-6.91 (m, 3H), 4.93 (dd, J = 13.5, 4.8 Hz, 1H), 3.28-3.12 (m, 1H), 2.84 |
| | (ddd, J = 16.4, 13.3, 4.1 Hz, 2H), 2.34 (s, 6H). |
| 452 | MS m/z 421.9 [M + H]⁺; ¹H NMR (400 MHz, DMSO) δ 11.14 (s, 1H), 7.51 (d, J = 2.1 Hz, |
| | 1H), 7.35 (s, 1H), 7.30 (d, J = 8.7 Hz, 1H), 7.09-7.07 (m, 1H), 7.07-7.04 (m, 1H), 6.95 (d, |
| | J = 7.9 Hz, 1H), 6.89 (t, J = 7.8 Hz, 1H), 4.91 (dd, J = 13.5, 4.7 Hz, 1H), 3.28-3.19 (m, |
| | 1H), 2.89-2.73 (m, 2H), 2.33 (s, 6H), 2.29 (s, 3H). |
| 453 | MS m/z 432.9 [M + H]⁺; ¹H NMR (400 MHz, DMSO) δ 11.13 (s, 1H), 7.92 (dd, |
| | J = 10.0, 1.5 Hz, 1H), 7.64 (dd, J = 8.0, 1.5 Hz, 1H), 7.55 (d, J = 2.0 Hz, 1H), 7.33 |
| | (d, J = 8.9 Hz, 2H), 7.24 (t, J = 7.7 Hz, 1H), 7.10 (dd, J = 8.6, 2.1 Hz, 1H), 4.94 |
| | (dd, J = 13.6, 4.7 Hz, 1H), 3.28-3.17 (m, 1H), 2.92-2.74 (m, 2H), 2.34 (s, 6H). |
| 459 | MS m/z 494.0 [M + H]⁺; (CDCl3, 400 MHz), δ: 8.01(s, 1H), 7.49-7.48(m, 1H), |
| | 7.42-7.37(m, 1H), 7.21-7.19(m, 1H), 7.17(s, 1H), 7.13-7.10(m.1H), 6.33-6.10(m, 1H), |
| | 5.29-5.25(m, 1H), 3.78-3.76(m, 4H), 3.49-3.46(m, 4H), 3.40-3.35(m, 1H), |
| | 2.91-2.86(m, 2H), 2.41(s, 6H). |
| 468 | MS m/z 442.0 [M + H]⁺; ¹H NMR (400 MHz, DMSO) δ 11.17 (s, 1H), 7.55 (d, |
| | J = 2.0 Hz, 1H), 7.51-7.46 (m, 1H), 7.33 (d, J = 8.6 Hz, 2H), 7.26-7.21 (m, 1H), |
| | 7.13-7.03 (m, 2H), 4.96 (dd, J = 13.4, 4.9 Hz, 1H), 3.30 (td, J = 13.2, 4.3 Hz, 1H), |
| | 3.00-2.70 (m, 2H), 2.39 (s, 6H). |
| 490 | MS m/z 531.0 [M + H]⁺; (DMSO-d, 400 MHz) 11.06 (s, 1H), 7.56 (s, 1H), 7.31-7.26 |
| | (m, 2H), 7.20 (s, 1H), 7.03 (t, J = 7.6 Hz, 1H), 6.96-6.91 (m, 1H), 6.82 (t, J = 6.8 Hz, |
| | 1H), 4.89 (q, J = 9.2 Hz, 1H), 3.59-3.49 (m, 1H), 2.98-2.83 (m, 2H), 2.26 (s, 3H). |
| 493 | MS m/z 463.0 [M + H]⁺; ¹H NMR (400 MHz, DMSO) δ 8.56(d, J = 4.8 Hz, 1H), |
| | 7.96(s, 1H), 7.31(d, J = 0.8 Hz, 1H), 7.24 (q, J = 4.4 Hz, 1H), 7.19(q, J = 6.8 Hz, |
| | 1H), 6.97-6.92 (m, 1H), 6.89-6.84 (m, 3H), 5.22 (q, J = 9.2 Hz, 1H), 358-3.51(m, |
| | 1H), 3.03-2.95(m, 1H), 2.88 (q, J = 12.4 Hz, 1H), 2.30(d, J = 1.6 Hz, 3H). |
| 513 | MS m/z 561.0 [M + H]⁺; ¹H (DMSO-d₆, 400 MHz) 11.23 (s, 1H), 8.79 (d, J = 4.0 Hz, |
| | 1H), 8.13 (q, J = 14.4 Hz, 2H), 8.06 (s, 2H), 7.80 (q, J = 6.8 Hz, 1H), 7.45 (q, J = 5.2 Hz, |
| | 2H), 7.11 (d, J = 4.8 Hz, 1H), 6.97 (s, 1H), 6.81 (d, J = 9.2 Hz, 1H), 5.00 (s, 1H), 3.46 |
| | (s, 4H), 3.38 (d, J = 13.2 Hz, 1H), 3.01 (q, J = 10.8 Hz, 1H), 2.93-2.85 (m, 1H), 2.39 (s, |
| | 4H), 2.22 (s, 3H). |

### Example 16A

### Preparation of Compound 523

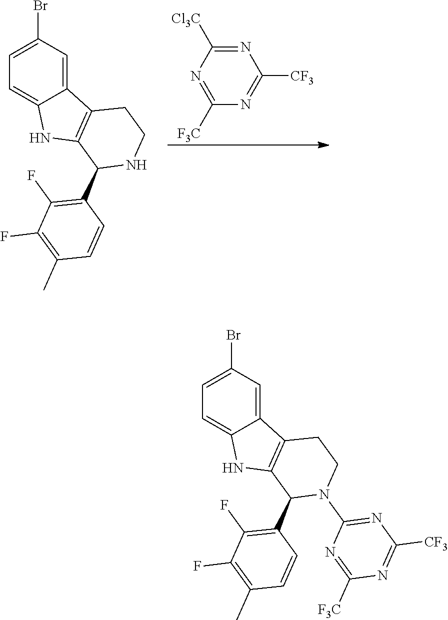

A 20 mL screw-cap vial equipped with a stir bar was charged with (1S)-6-bromo-1-(2,3-difluoro-4-methyl-phenyl)-2,3,4,9-tetrahydro-1H-pyrido[3,4-b]indole (450 mg, 1.19 mmol), DMAP (190 mgs, 1.48 mmol) and acetonitrile (3 mL), capped and placed under an argon atmosphere. Then a solution of 2-(trichloromethyl)-4,6-bis(trifluoromethyl)-1,3,5-triazine (550 mg, 1.64 mmol) in acetonitrile (2 mL) was added via syringe. The reaction mixture was stirred at ambient temperature for 15 min, at which point reaction monitoring by LCMS indicated consumption of starting material and formation of the desired product. The mixture was partitioned between EtOAc and water, the organic phase was washed with brine, dried over MgSO₄ and then purified by silca-gel chromatography, eluting with Hexanes:EtOAc (0-20% gradient) to afford Compound 523 (550 mg, 77.8% yield) as an off white amorphous solid. MS m/z 589.9 [M−H]⁻; ¹H NMR (DMSO-d₆, 400 MHz) δ: 11.20 (s, 1), 7.74 (s, 1), 7.3-7.3 (m, 1H), 7.2-7.2 (m, 2H), 7.0-7.2 (m, 1H), 6.86 (t, J=7.3 Hz, 1H), 4.95 (br dd, J=4.8, 13.3 Hz, 1H), 3.5-3.6 (m, 1H), 3.0-3.1 (m, 1H), 2.8-3.0 (m, 1H), 2.27 (s, 3H).

Using the procedure described for Compound 523 above, additional compounds described herein were prepared by substituting the appropriate starting materials, suitable reagents and reaction conditions, obtaining compounds such as those selected from:

| Cpd | Data |
|---|---|
| 298 | MS m/z 561.9[M + H]⁺; ¹H NMR (DMSO-d₆) δ: 10.95-11.10 (m, 1H), 8.66-8.80 |
| | (m, 1H), 8.27 (s, 2H), 7.62 (d, J = 1.8 Hz, 1H), 7.21 (d, J = 8.5 Hz, 1H), 7.13 (dd, |
| | J = 8.5, 1.8 Hz, 1H), 7.05 (d, J = 4.9 Hz, 1H), 6.74 (br s, 1H), 4.78-5.06 (m, 1H), |
| | 3.49-3.65 (m, 8H), 3.30-3.39 (m, 1H), 2.83-2.92 (m, 1H), 2.67-2.79 (m, 1H) |
| 299 | MS m/z 521.0 [M + H]⁺; ¹H NMR (DMSO-d₆) δ: 11.11 (s, 1H), 8.33 (s, 2H), |
| | 7.67-7.70 (m, 1H), 7.28 (d, J = 8.5 Hz, 1H), 7.20 (dd, J = 8.5, 1.8 Hz, 1H), 6.92 |
| | (s, 1H), 5.03 (s, 1H), 3.59-3.70 (m, 8H), 3.24-3.32 (m, 1H), 2.92 (dd, J = 15.6, 3.4 |
| | Hz, 1H), 2.69-2.82 (m, 1H), 2.41 (s, 3H), 2.34 (s, 3H) |
| 387 | MS m/z 548.0 [M + H]⁺; ¹H NMR (DMSO-d₆) δ: 11.19 (s, 1H), 7.59 (d, J = 1.6 Hz, |
| | 1H), 7.34 (d, J = 8.8 Hz, 1H), 7.23 (s, 1H), 7.05-7.12 (m, 2H), 6.84-6.88 (m, 1H), |
| | 4.93-4.97 (m, 1H), 3.53-3.59 (m, 1H), 2.91-3.06 (m, 2H), 2.27 (d, J = 1.6 Hz, 3H) |
| 405 | MS m/z 584.1 [M + H]⁺; ¹H NMR (DMSO-d₆) δ: 11.18 (s, 1H), 8.14 (d, J = 2.4 Hz, |
| | 1H), 7.58 (d, J = 2.0 Hz, 1H), 7.47-7.40 (m, 1H), 7.33 (d, J = 8.4 Hz, 1H), 7.12-7.09 |
| | (m, 1H), 6.96 (s, 1H), 6.83 (d, J = 8.8 Hz, 1H), 4.96-4.91 (m, 1H), 3.66 (t, J = 4.8 |
| | and J = 10.0 Hz, 4H), 3.51-3.42 (m, 5H), 3.05-3.00 (m, 1H), 2.92-2.83 (m, 1H) |
| 420 | MS m/z 598.2 [M + H]⁺; ¹H NMR (DMSO-d₆) δ: 11.13 (s, 1H), 8.31 (s, 2H), 7.59 |
| | (s, 1H), 7.35 (d, J = 8.8 Hz, 1H), 7.11 (dd, J = 8.4 Hz, 2.0 Hz, 1H), 6.85 (s, 1H) |
| | 4.99-4.94 (m, 1H), 3.72 (s, 4H), 3.58-3.50 (m, 1H), 3.06-3.01 (m, 1H), 2.90-2.81 |
| | (m, 1H), 2.51 (s, 4H), 2.20 (s, 3H) |
| 461 | MS m/z 480.0 [M + H]⁺; ¹H NMR (DMSO-d₄) δ: 11.20 (s, 1H), 8.96 (d, J = 13.6 Hz, |
| | 1H), 7.57(t, J = 2.8 Hz, 1H), 7.36-7.22 (m, 2H), 6.12-7.04 (m, 2H), 6.81 (t, J = 6.8 |
| | Hz, 1H), 5.02-4.87(m, 1H), 3.50-3.42 (m, 1H), 3.01-2.95 (m, 1H), 2.92-2.83 |
| | (m, 1H), 2.27 (s, 3H) |
| 504 | MS m/z 538.0 [M + H]⁺; ¹H NMR (DMSO-d₆, 400 MHz) 11.22-11.19 (m, 1H), 7.58 |
| | (s, 1H), 7.53-7.21 (m, 2H), 7.12-7.05 (m, 2H), 6.85 (t, J = 6.8 Hz, 1H), 5.04-4.89 |
| | (m, 1H), 3.94 (d, J = 8.8 Hz, 3H), 3.57-3.45 (m, 1H), 3.05-2.86 (m, 2H), 2.27 (s, 3H). |
| 505 | MS m/z 596.0 [M + H]⁺; ¹H NMR (DMSO-d₆, 400 MHz) 11.26-11.20 (m, 1H), 7.58 |
| | (s, 1H), 7.35-7.21 (m, 2H), 7.12-7.07 (m, 2H), 6.93-6.83 (M, 1H), 5.04-4.92 (m, |
| | 1H), 3.58-3.51 (m, 1H), 3.05-2.86 (m, 2H), 2.27 (s, 3H). |
| 507 | MS m/z 567.1[M + H]⁺; ¹H NMR (400 MHz, DMSO) δ 11.12 (d, J = 11.9 Hz, 1H), |
| | 8.35 (d, J = 12.7 Hz, 2H), 7.58 (d, J = 1.7 Hz, 1H), 7.34 (d, J = 8.6 Hz, 1H), 7.10 |
| | (dd, J = 8.5, 6.4 Hz, 1H), 6.97-6.68 (m, 2H), 4.99 (ddd, J = 24.6, 13.5, 5.0 Hz, |
| | 1H), 3.71-3.65 (m, 4H), 3.64-3.60 (m, 4H), 3.52 (ddd, J = 17.3, 8.9, 4.3 Hz, 1H), |
| | 3.03 (d, J = 15.5 Hz, 1H), 2.85 (t, J = 11.1 Hz, 1H). |
| 508 | MS m/z 580.2[M + H]⁺; ¹H NMR (400 MHz, DMSO) δ 11.11 (d, J = 11.6 Hz, 1H), |
| | 8.32 (d, J = 12.6 Hz, 2H), 7.58 (d, J = 1.8 Hz, 1H), 7.33 (d, J = 8.6 Hz, 1H), |
| | 7.12-7.04 (m, 1H), 6.97-6.67 (m, 2H), 4.99 (ddd, J = 25.0, 13.6, 5.2 Hz, 1H), |
| | 3.78-3.65 (m, 4H), 3.57-3.45 (m, 1H), 3.02 (d, J = 15.4 Hz, 1H), 2.84 |
| | (t, J = 11.2 Hz, 1H), 2.35-2.27 (m, 4H), 2.18 (s, 3H). |
| 510 | MS m/z 530.1[M + H]⁺; ¹H NMR (400 MHz, DMSO) δ 11.19 (d, J = 9.1 Hz, 1H), |
| | 7.58 (t, J = 2.1 Hz, 1H), 7.33 (dd, J = 8.6, 1.9 Hz, 1H), 7.26 (d, J = 29.2 Hz, 1H), |
| | 7.13-7.03 (m, 2H), 6.84 (ddd, J = 53.2, 51.9, 11.1 Hz, 2H), 4.96 (ddd, J = 26.7, |
| | 13.3, 4.9 Hz, 1H), 3.52 (qd, J = 12.9, 4.3 Hz, 1H), 3.08-2.96 (m, 1H), 2.96-2.84 |
| | (m, 1H), 2.27 (s, 3H). |
| 522 | MS m/z 530.1 [M + H]⁺; ¹H NMR (DMSO-d₄) δ: 11.12 (s, 1H), 8.57(s, 2H), 7.60(d, |
| | J = 1.6 Hz, 1H), 7.36 (d, J = 8.4 Hz, 1H), 7.12 (dd, J = 8.8, 2.0 Hz, 1H), 6.96 (s, 1H), |
| | 5.00-4.96 (m, 1H), 3.90 (s, 3H), 3.61-3.54 (m, 1H), 3.07-3.03 (m, 1H), 2.92-2.83 |
| | (m, 1H) |
| 527 | MS m/z 557.2 [M − H]⁻; ¹H NMR (400 MHz, MeOD) δ: 8.53 (d, J = 2.3 Hz, 1H), |
| | 8.04 (dd, J = 9.0, 2.3 Hz, 1H), 7.40 (d, J = 9.1 Hz, 1H), 7.36 (s, 1H), 7.00 (t, J = 7.5 |
| | Hz, 1H), 6.89 (t, J = 7.3 Hz, 1H), 5.18 (dd, J = 13.5, 5.0 Hz, 1H), 3.66 (ddd, |
| | J = 13.2, 11.2, 5.0 Hz, 1H), 3.19-3.01 (m, 2H), 2.29 (d, J = 2.3 Hz, 3H). |
| 529 | MS m/z 592.3 [M − H]⁻; ¹H NMR (400 MHz, MeOD) δ: 7.92 (s, 1H), 7.76 (s, 1H), |
| | 7.00 (t, J = 7.7 Hz, 1H), 6.86 (t, J = 7.3 Hz, 1H), 5.15 (d, J = 13.7 Hz, 1H), 3.63 |
| | (dd, J = 14.0, 6.9 Hz, 1H), 3.08 (d, J = 7.4 Hz, 2H), 2.30 (s, 3H), 2.18 (s, 3H). |
| 531 | MS m/z 586.2[M + H]⁺; ¹H NMR (400 MHz, DMSO) δ 11.15 (s, 1H), 8.21 (s, 2H), |
| | 7.58 (d, J = 2.0 Hz, 1H), 7.35 (d, J = 8.4 Hz, 1H), 7.23 (t, J = 5.6 Hz, 1H), 7.11 |
| | (dd, J = 8.8 Hz, 2.0 Hz, 1H), 6.83 (s, 1H), 4.96 (dd, J = 13.6 Hz, 5.2 Hz, 1H), |
| | 3.57-3.50 (m, 1H), 3.05-3.00 (m, 1H), 2.89-2.81 (m, 1H), 2.51-2.50 (m, 7H), 2.38 |
| | (t, J = 6.8 Hz, 2H), 1.23 (s, 1H). |
| 532 | MS m/z 558.2[M + H]⁺; ¹H NMR (400 MHz, DMSO-d₆) δ 11.11 (s, 1H), 8.98 (s, |
| | 2H), 7.64 (d, 1H, J = 2.0 Hz), 7.39 (d, 1H, J = 8.8 Hz), 7.14 (dd, 1H, J = 8.4 Hz, 2.0 |
| | Hz), 7.05 (s, 1H), 5.04-4.98 (m, 1H), 3.91 (s, 3H), 3.68-3.61 (m, 1H), 3.11-3.08 |
| | (m, 1H), 2.96-2.92 (m, 1H) |
| 533 | MS m/z 542.1[M + H]⁺; ¹H NMR (400 MHz, DMSO-d₆) δ 11.14 (s, 1H), 9.00 (s, |
| | 2H), 7.64 (d, 1H, J = 2.0 Hz), 7.39 (d, 1H, J = 8.4 Hz), 7.14 (dd, 1H, J = 8.8 Hz, 2.0 |
| | Hz), 7.07 (s, 1H), 5.03-4.98 (m, 1H), 3.65-3.62 (m, 1H), 3.11-3.06 (m, 1H), |
| | 2.92-2.88 (m, 1H), 2.66 (s, 3H) |
| 536 | MS m/z 574.1[M + H]⁺; ¹H NMR (400 MHz, DMSO) δ 11.12 (s, 1H), 8.56 (s, |
| | 2H), 7.60 (d, J = 1.6 Hz, 1H), 7.36 (d, J = 8.8 Hz, 1H), 7.12 (q, J = 8.4 Hz, 1H), 6.95 |
| | (s, 1H), 4.98 (q, J = 13.6 Hz, 1H), 4.40 (q, J = 4.0 Hz, 2H), 3.64 (q, J = 4.0 Hz, 2H), |
| | 3.54-3.60 (m, 1H), 3.28 (s, 3H), 3.05 (q, J = 16.0 Hz, 1H), 2.82-2.92 (m, 1H). |

### Example 16B

### Preparation of Compound 524

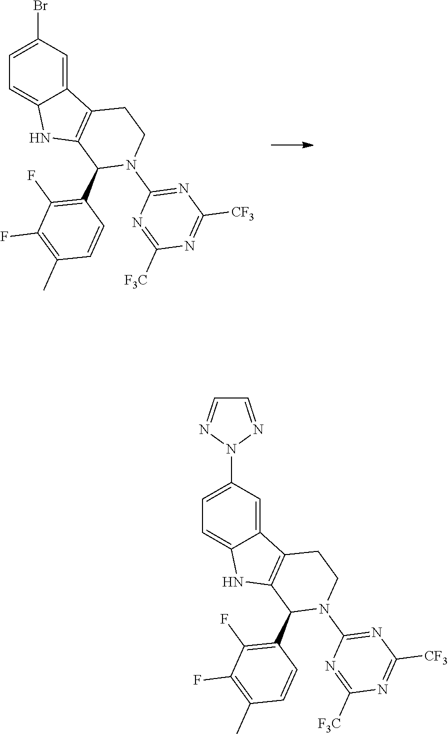 [SMILES: Cc1ccc([C@H]2c3[nH]c4ccc(Br)cc4c3CCN2c2nc(C(F)(F)F)nc(C(F)(F)F)n2)c(F)c1F ; Cc1ccc([C@H]2c3[nH]c4ccc(-n5nccn5)cc4c3CCN2c2nc(C(F)(F)F)nc(C(F)(F)F)n2)c(F)c1F]

An 8 mL screw-cap vial with a septum cap was charged with tris(dibenzylideneacetone)dipalladium(0) (10 mg, 0.01 mmol), 2-di-tert-butylphosphino-3,4′5′-tetramethyl-2′,4′,6′-triisopropyl-1,1′-biphenyl (12 mg, 0.02 mmol) and toluene (1 ml), the mixture was degassed and backfilled with argon (3×), placed in a heating block pre-heated to 105° C. and allowed to stir for 2 min. The dark solution was transferred by syringe to another 8 mL screw cap vial containing an argon sparged mixture of Compound 523 (1S)-2-[4,6-bis(trifluoromethyl)-1,3,5-triazin-2-yl]-6-bromo-1-(2,3-difluoro-4-methyl-phenyl)-1,3,4,9-tetrahydropyrido[3,4-b]indole (102 mg, 0.17 mmol), 2H-triazole (18 mg, 0.26 mmol), potassium phosphate tribasic (78 mg, 0.36 mmol) suspended in toluene (1 ml) and 1,4-dioxane (0.2 mL). The resulting mixture was allowed to stir at 105° C. for 2 h. After being allowed to cool to ambient temperature, the mixture was partitioned between EtOAc and water, the organic phase washed with brine, dried over MgSO₄, and evaporated to dryness under reduced pressure. The dark oily residue was subjected to silica gel chromatography, eluting with hexanes EtOAc (gradient 0-40%) to afford Compound 524 (72 mg, 72.0% yield) as a white solid. MS m/z 581.0 [M+H]⁻; ¹H NMR (DMSO-d₆, 400 MHz) δ: 11.28 (s, 1H), 8.16 (d, J=1.3 Hz, 1H), 8.07 (s, 2H), 7.8-7.9 (m, 1H), 7.49 (d, J=8.8 Hz, 1H), 7.26 (s, 1H), 7.09 (br t, J=7.5 Hz, 1H), 6.92 (br t, J=7.3 Hz, 1H), 4.9-5.1 (m, 1H), 3.5-3.7 (m, 1H), 3.1-3.2 (m, 1H), 2.9-3.1 (m, 1H), 2.2-2.3 (m, 3H).

Using the procedure described for Compound 524 above, additional compounds described herein below were prepared by substituting the appropriate starting materials, suitable reagents and reaction conditions, obtaining compounds such as those selected from:

| Cpd | Data |
|---|---|
| 287 | MS m/z 476.1 [M + H]⁺; ¹H NMR (DMSO-d₆) δ: 11.31 (br s, 1H), 8.72-8.88 (m, |
| | 1H), 8.11 (s, 1H), 8.06 (s, 2H), 7.80 (dd, J = 8.5, 1.8 Hz, 1H), 7.46 (d, J = 8.9 Hz, |
| | 1H), 7.22-7.29 (m, 2H), 7.18 (d, J = 7.9 Hz, 2H), 7.04-7.13 (m, 2H), 4.73-5.16 (m, |
| | 1H), 3.33 (m, 1H), 2.83-3.04 (m, 2H), 2.28 (s, 3H) |
| 288 | MS m/z 549.4 [M + H]⁺; ¹H NMR (DMSO-d₆) δ: 11.15-11.30 (m, 1H), 8.80 (br d, |
| | J = 4.6 Hz, 1H), 8.39 (s, 2H), 8.13 (d, J = 1.8 Hz, 1H), 8.06 (s, 2H), 7.81 (dd, J = 8.9, |
| | 2.1 Hz, 1H), 7.47 (d, J = 8.9 Hz, 1H), 7.13 (d, J = 4.6 Hz, 1H), 6.85 (br s, 1H), |
| | 4.97-5.17 (m, 1H), 3.64-3.75 (m, 4H), 3.58-3.64 (m, 4H), 3.41-3.51 (m, 1H), |
| | 2.99-3.09PTC-0421550 (m, 1H), 2.83-2.96 (m, 1H) |
| 300 | MS m/z 510.2 [M + H]⁺; ¹H NMR (DMSO-d₆) δ: 11.23 (s, 1H), 8.39 (s, 2H), 8.13 (d, |
| | J = 1.8 Hz, 1H), 8.07 (s, 2H), 7.82 (dd, J = 8.7, 2.0 Hz, 1H), 7.48 (d, J = 8.5 Hz, 1H), 7.00 |
| | (s, 1H), 5.10 (br dd, J = 13.3, 5.0 Hz, 1H), 3.56-3.77 (m, 8H), 3.36-3.50 (m, 1H), 3.07 (br |
| | dd, J = 15.3, 3.1 Hz, 1H), 2.80-2.92 (m, 1H), 2.48-2.54 (s, 3H), 2.37-2.47 (m, 3H) |
| 301 | MS m/z 548.1 [M + H]⁺; ¹H NMR (DMSO-d₆) δ: 11.05 (s, 1H), 8.80 (br d, J = 4.6 |
| | Hz, 1H), 8.42 (d, J = 2.4 Hz, 1H), 8.37 (s, 2H), 7.91 (d, J = 1.8 Hz, 1H), 7.64-7.78 |
| | (m, 1H), 7.58 (dd, J = 8.7, 2.0 Hz, 1H), 7.37-7.44 (m, 1H), 7.13 (d, J = 4.9 Hz, 1H), |
| | 6.51 (t, J = 2.0 Hz, 1H), 6.26 (t, J = 1.7 Hz, 1H), 4.95-5.14 (m, 1H), 3.60-3.73 (m, |
| | 8H), 3.51-3.51 (m, 1H), 2.96-3.06 (m, 1H), 2.81-2.94 (m, 1H) |
| 314 | MS m/z 547.0 [M + H]⁺; ¹H NMR (DMSO-d₆) δ: 11.00 (br s, 1H), 8.81 (br s, 1H), 8.37 |
| | (br s, 2H), 7.63 (br s, 1H), 7.37 (br d, J = 8.2 Hz, 1H), 7.24-7.32 (m, 3H), 7.09-7.24 |
| | (m, 1H), 6.77-6.92 (m, 1H), 6.24 (br s, 2H), 5.04 (br d, J = 1.8 Hz, 1H), 3.55-3.79 |
| | (m, 8H), 3.22-3.50 (m, 1H), 3.00 (br d, J = 14.6 Hz, 1H), 2.76-2.93 (m, 1H) |
| 316 | MS m/z 548.9 [M + H]⁺; ¹H NMR (DMSO-d₆) δ: 11.06-11.39 (s, 1H), 8.80 (br s, |
| | 1H), 7.94-8.34 (m, 4H), 7.81 (br d, J = 8.5 Hz, 1H), 7.47 (br t, J = 10.7 Hz, 2H), 7.11 |
| | (br d, J = 4.3 Hz, 1H), 6.91-7.08 (m, 1H), 6.74-6.91 (m, 1H), 4.76-5.21 (m, 1H), |
| | 3.19-3.88 (m, 9H), 2.77-3.12 (m, 2H) |
| 454 | MS m/z 616.7 [M + H]⁺; ¹H NMR (DMSO-d₆, 400 MHz) δ: 11.22 (s, 1H), 8.19 (s, |
| | 1H), 8.14 (s, 1H), 8.06 (s, 2H), 7.81 (dd, J = 2.0, 8.8 Hz, 1H), 7.54 (s, 1H), 7.4-7.5 |
| | (m, 2H), 6.93 (s, 1H), 6.82 (d, J = 8.8 Hz, 1H), 4.9-5.0 (m, 1H), 4.0-4.1 (m, 1H), |
| | 3.6-3.7 (m, 4H), 3.4-3.5 (m, 4H), 3.0-3.1 (m, 1H), 2.9-3.0 (m, 1H) |
| 482 | MS m/z 617.0 [M + H]⁺; ¹H NMR (DMSO-d₆) δ: 11.09-11.16 (s, 1H), 8.38 (s, 2H), |
| | 8.10-8.18 (m, 1H), 8.06 (s, 2H), 7.81 (dd, J = 8.6, 2.1 Hz, 1H), 7.56 (s, 1H), |
| | 7.43-7.52 (m, 1H), 6.80 (s, 1H), 5.02 (br dd, J = 13.3, 5.0 Hz, 1H), 3.44-3.76 |
| | (m, 9H), 3.06-3.18 (m, 1H), 2.87-3.01 (m, 1H) |
| 483 | MS m/z 631.0 [M + H]⁺; ¹H NMR (DMSO-d₆) δ: 11.13 (s, 1H), 8.35-8.37 (m, 1H), |
| | 8.38 (s, 1H), 8.07 (d, J = 1.9 Hz, 1H), 7.70-7.84 (m, 2H), 7.55 (s, 1H), 7.44 (d, |
| | J = 8.8 Hz, 1H), 6.79 (s, 1H), 5.01 (br dd, J = 13.4, 4.9 Hz, 1H), 3.49-3.75 (m, 9H), |
| | 3.00-3.18 (m, 1H), 2.84-2.99 (m, 1H), 2.37 (s, 3H) |
| 485 | MS m/z 616.7 [M + H]⁺; ¹H NMR (DMSO-d₆, 400 MHz) δ: 11.22 (s, 1H), 8.19 (d, |
| | J = 2.3 Hz, 1H), 8.14 (d, J = 1.8 Hz, 1H), 8.06 (s, 2H), 7.81 (dd, J = 1.8, 8.8 Hz, 1H), 7.54 |
| | (s, 1H), 7.4-7.5 (m, 2H), 6.93 (s, 1H), 6.82 (d, J = 8.8 Hz, 1H), 4.9-5.0 (m, 1H), 3.6-3.7 |
| | (m, 4H), 3.4-3.6 (m, 1H), 3.4-3.4 (m, 4H), 3.0-3.1 (m, 1H), 2.9-3.0 (m, 1H) |
| 486 | MS m/z 617.6 [M + H]⁺; ¹H NMR (DMSO-d₆, 400 MHz) δ: 11.27 (s, 1H), 8.15 (br |
| | s, 2H), 8.07 (s, 2H), 7.83 (dd, J = 1.8, 8.8 Hz, 1H), 7.4-7.6 (m, 2H), 7.00 (s, 1H), |
| | 6.8-7.0 (m, 1H), 4.97 (br s, 1H), 3.6-3.7 (m, 4H), 3.56-3.49 (m, 1H), 3.4-3.5 (m, |
| | 4H), 3.1-3.2 (m, 1H), 2.9-3.0 (m, 1H) |
| 500 | MS m/z 630.9 [M + H]⁺; ¹H NMR (DMSO-d₆, 400 MHz) δ: 11.18 (s, 1H), 9.8-10.0 |
| | (m, 1H), 8.46 (s, 2H), 8.15 (d, J = 1.8 Hz, 1H), 8.07 (s, 2H), 7.82 (dd, J = 2.0, 8.8 |
| | Hz, 1H), 7.59 (s, 1H), 7.49 (d, J = 8.8 Hz, 1H), 6.84 (s, 1H), 5.02 (br dd, J = 5.3, |
| | 13.3 Hz, 1H), 4.69 (br d, J = 14.3 Hz, 2H), 3.5-3.7 (m, 1H), 3.49 (br d, J = 11.3 |
| | Hz, 2H), 3.2-3.3 (m, 2H), 3.1-3.2 (m, 1H), 2.9-3.1 (m, 3H), 2.83 (s, 3H) |
| 501 | MS m/z 631.1 [M + H]⁺; ¹H NMR (DMSO-d₆) δ: 11.18-11.28 (s, 1H), 8.45 (s, 2H), |
| | 8.16 (d, J = 2.0 Hz, 1H), 8.08 (s, 2H), 7.84 (dd, J = 8.8, 2.0 Hz, 1H), 7.51 (d, J = 8.8 |
| | Hz, 1H), 6.93 (s, 1H), 5.02 (br dd, J = 13.0, 5.3 Hz, 1H), 4.70 (br d, J = 13.5 Hz, 2H), |
| | 3.42-3.69 (m, 4H), 2.89-3.33 (m, 6H), 2.84 (br s, 3H) |
| 502 | MS m/z 512.0 [M + H]⁺; ¹H NMR (DMSO-d, 400 MHz) δ: 11.25 (s, 1H), 8.79 (d, |
| | J = 4.8 Hz, 1H), 8.12 (d, J = 4.0 Hz, 1H), 8.05 (s, 2H), 7.81 (q, J = 6.4 Hz, 1H), 7.46 |
| | (d, J = 8.8 Hz, 1H), 7.33 (s, 1H), 7.13 (d, J = 5.2 Hz, 1H), 7.03 (t, J = 7.2 Hz, 1H), 6.81 |
| | (t, J = 6.8 Hz, 1H), 4.94 (d, J = 11.6 Hz, 1H), 3.47-3.40 (m, 1H), 3.01-2.87 (m, 2H), |
| | 2.26 (d, J = 1.6 Hz, 3H). |
| 506 | MS m/z 563.8 [M + H]⁺; ¹H NMR (DMSO-d₆) δ: 11.11-11.29 (s, 1H), 8.96 (d, J = 19.8 |
| | Hz, 1H), 8.46 (d, J = 5.8 Hz, 2H), 8.15 (s, 1H), 8.07 (s, 2H), 7.77-7.92 (m, 1H), 7.50 (dd, |
| | J = 8.8, 3.5 Hz, 1H), 6.78-7.06 (m, 1H), 4.91-5.22 (m, 1H), 4.70 (br d, J = 13.3 Hz, 2H), |
| | 3.40-3.65 (m, 3H), 3.23 (br t, J = 12.8 Hz, 2H), 2.88-3.15 (m, 5H), 2.55 (s, 3H) |
| 514 | MS m/z 617.1 [M + H]⁺; ¹H NMR (DMSO-d₆, 400 MHz) δ: 11.17 (s, 1H), 8.38 (s, |
| | 2H), 8.1-8.2 (m, 1H), 8.06 (s, 2H), 7.81 (dd, J = 2.1, 8.9 Hz, 1H), 7.55 (s, 1H), |
| | 7.4-7.5 (m, 1H), 6.80 (s, 1H), 5.01 (br dd, J = 5.1, 13.4 Hz, 1H), 3.5-3.8 (m, 9H), |
| | 3.0-3.2 (m, 1H), 2.8-3.0 (m, 1H) |
| 515 | MS m/z 617.1 [M + H]⁺; ¹H NMR (DMSO-d₆) δ: 11.13-11.31 (m, 1H), 8.90-9.00 |
| | (m, 1H), 8.39 (d, J = 6.0 Hz, 2H), 8.14 (s, 1H), 8.07 (s, 2H), 7.82 (br d, J = 8.8 Hz, |
| | 1H), 7.49 (dd, J = 8.8, 2.8 Hz, 1H), 6.80-7.03 (m, 1H), 4.92-5.16 (m, 1H), 3.59-3.86 |
| | (m, 8H), 3.39-3.59 (m, 1H), 2.83-3.33 (m, 2H) |
| 516 | MS m/z 616.2.0 [M − H]⁻; ¹H NMR (DMSO-d₆) δ: 11.12-11.25 (s, 1H), 8.37 (s, 2H), |
| | 8.15 (d, J = 2.0 Hz, 1H), 8.07 (s, 2H), 7.83 (dd, J = 8.9, 2.1 Hz, 1H), 7.49 (d, J = 8.8 |
| | Hz, 1H), 6.89 (s, 1H), 5.01 (dd, J = 13.6, 5.1 Hz, 1H), 3.50-3.77 (m, 9H), 3.08-3.21 |
| | (m, 1H), 2.84-3.06 (m, 1H) |
| 519 | MS m/z 580.0 [M + H]⁺; ¹H NMR (DMSO-d₆, 400 MHz) δ: 11.25 (s, 1H), 8.14 (s, 1H), |
| | 8.06 (s, 2H), 7.8-7.8 (m, 1H), 7.57 (s, 1H), 7.4-7.5 (m, 1H), 7.22 (s, 1H), 7.0-7.1 |
| | (m, 1H), 6.88 (t, J = 7.4 Hz, 1H), 4.9-5.0 (m, 1H), 3.5-3.6 (m, 1H), 3.0-3.1 (m, |
| | 1H), 2.9-3.0 (m, 1H), 2.2-2.3 (m, 3H) |
| 520 | MS m/z 594.0 [M + H]⁺; ¹H NMR (DMSO-d₆) δ: 11.21 (s, 1H), 8.07 (s, 1H), 7.81 (s, 1H), |
| | 7.76 (dd, J = 8.8, 1.8 Hz, 1H), 7.57 (s, 1H), 7.43 (d, J = 8.8 Hz, 1H), 7.22 (s, 1H), |
| | 7.05 (br t, J = 7.3 Hz, 1H), 6.87 (t, J = 7.1 Hz, 1H), 4.88-4.98 (m, 1H), 3.47-3.63 (m, |
| | 1H), 3.01-3.12 (m, 1H), 2.97 (br dd, J = 11.3, 5.0 Hz, 1H), 2.37 (s, 3H), 2.27 (s, 3H) |
| 525 | MS m/z 595.4 [M + H]⁺; ¹H NMR (DMSO-d₆) δ: 11.15-11.28 (s, 1H), 8.09 (s, 1H), 7.82 |
| | (s, 1H), 7.78 (dd, J = 8.9, 1.9 Hz, 1H), 7.45 (d, J = 8.8 Hz, 1H), 7.26 (s, 1H), 7.09 (br t, |
| | J = 7.4 Hz, 1H), 6.91 (br t, J = 7.0 Hz, 1H), 4.99 (br dd, J = 13.4, 5.1 Hz, 1H), 3.53-3.68 |
| | (m, 1H), 3.07-3.19 (m, 1H), 2.91-3.05 (m, 1H), 2.38 (s, 3H), 2.28 (s, 3H) |
| 528 | MS m/z 541.2 [M − H]⁻; ¹H NMR (400 MHz, MeOD) δ: 7.30 (s, 1H), 7.27 (s, 1H), 7.18 |
| | (d, J = 8.9 Hz, 1H), 6.94 (t, J = 7.4 Hz, 1H), 6.83-6.71 (m, 2H), 5.10-4.99 (m, 1H), 3.83 |
| | (d, J = 1.6 Hz, 3H), 3.63-3.50 (m, 1H), 2.96 (d, J = 14.8 Hz, 2H), 2.28 (d, J = 2.2 Hz, 3H). |
| 530 | MS m/z 594.2 [M + H]⁺; ¹H NMR (400 MHz, DMSO) δ: 11.10 (s, 1H), 8.27 (d, J = 2.3 |
| | Hz, 1H), 7.87 (d, J = 2.1 Hz, 1H), 7.54 (dd, J = 8.7, 2.1 Hz, 1H), 7.37 (d, J = 8.7 Hz, 1H), |
| | 7.24 (s, 1H), 7.07 (t, J = 7.5 Hz, 1H), 6.89 (t, J = 7.4 Hz, 1H), 6.28 (d, J = 2.3 Hz, 1H), |
| | 4.97 (dd, J = 13.4, 5.3 Hz, 1H), 3.65-3.53 (m, 1H), 3.09 (dd, J = 15.2, 4.1 Hz, 1H), 2.96 |
| | (ddd, J = 16.1, 11.6, 5.5 Hz, 1H), 2.27 (s, 6H). |

### Example 16C

### Preparation of Compound 406

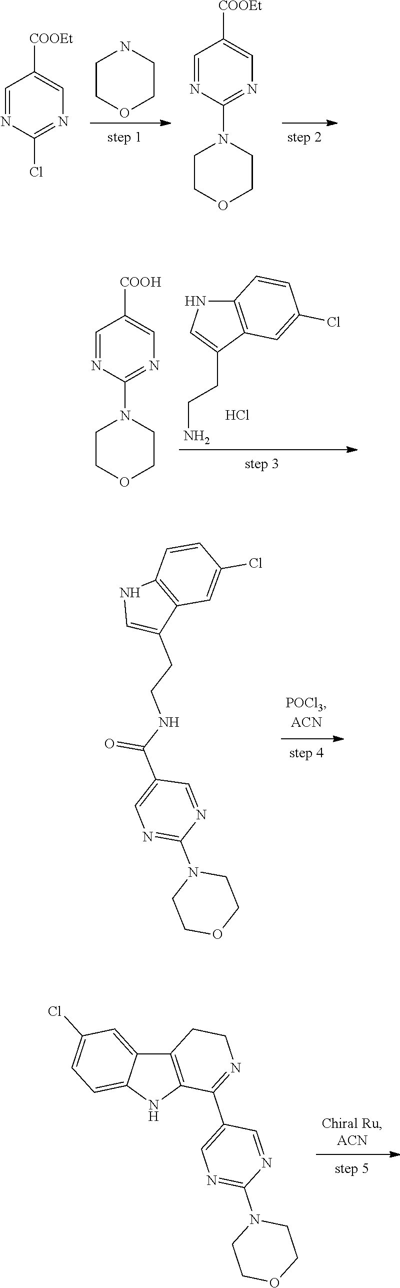

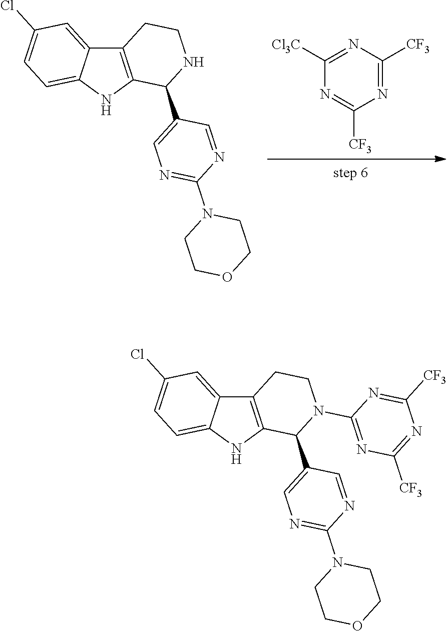

Step 1: Ethyl 2-chloropyrimidine-5-carboxylate (25.0 g, 134 mmol) was dissolved in CH₃CN (100 mL). Then K₂CO₃ (55.5 g, 402 mmol) and morpholine (12.3 g, 141 mmol) were added. The mixture was refluxed for 6 h and cooled to RT. The mixture was concentrated to give a residue. The residue was suspended in water (150 mL) and extracted with ethyl acetate (3*150 mL). The organic phase was dried over Na₂SO₄ and filtered. The filtrate was concentrated and purified through combi-flash (eluent: gradient, from 100% petroleum ether to petroleum ether:ethyl acetate=4:1) to give ethyl 2-morpholinopyrimidine-5-carboxylate (31.5 g, 99.1% yield) as a pale yellow solid. ¹H NMR (400 MHz, CDCl₃) δ 8.87 (s, 1H), 4.36 (q, 2H, J=7.2 Hz), 3.95 (q, 4H, J=3.2 Hz), 3.78 (q, 4H, J=3.2 Hz), 1.38 (t, 3H, J=7.2 Hz).

Step 2: Ethyl 2-morpholinopyrimidine-5-carboxylate (31.5 g, 133 mmol) was dissolved in THF (50 mL) and water (50 mL), then LiOH (10 g, 409.22 mmol) was added. The mixture was stirred at 35° C. for 3 h. The mixture was adjusted pH to 7 with HCl aqueous solution (2 N), then concentrated in vacuo to remove THF. The residue was adjusted pH to 3-4 with HCl aqueous solution (2 N). The suspension was filtered. The filter cake was collected and dried in vacuo to get 2-morpholinopyrimidine-5-carboxylic acid (27 g, 97.2% yield) as white solid. LCMS: ESI-MS: m/z: 210.1 [M+H]⁺, RT=1.367 min. ¹H NMR (400 MHz, DMSO-d₆) δ 12.87 (s, 1H), 8.78 (s, 2H), 3.83 (t, J=5.0 Hz, 4H), 3.67 (t, J=5.0 Hz, 4H).

Step 3: 2-Morpholinopyrimidine-5-carboxylic acid (27 g, 129.06 mmol) and 2-(5-chloro-1H-indol-3-yl)ethanamine hydrochloride (33 g, 142.78 mmol) were combined and dissolved in DMF (300 mL). Then DIPEA (80 mL, 500 mmol) was added. After that, HATU (75 g, 193.303 mmol) was added. The mixture was stirred at 25° C. for 20 h. The mixture was poured into a mixture of EtOAc (250 mL) and water (150 mL). The mixture was seperated and the aqueous phase was extracted with EtOAc (2*100 mL). The organic phase was washed with brine (4*100 mL) and dried over Na₂SO₄, then filtered. The filtrate was concentrated in vacuo to give a residue, The residue was suspended in EtOAc (50 mL), then filtered. The filter cake was collected and dried in vacuo to get N-[2-(5-chloro-1H-indol-3-yl)ethyl]-2-morpholino-pyrimidine-5-carboxamide (38 g, 76.32% yield) as a white solid. LCMS: ESI-MS: m/z: 386.1 [M+H]⁺, RT=1.774 min. ¹H NMR (400 MHz, DMSO-d₆) δ 11.03 (s, 1H), 8.78 (s, 2H), 8.51-8.48 (m, 1H), 7.59 (d, J=2.0 Hz, 1H), 7.35 (d, J=4.4 Hz, 1H), 7.26 (d, J=2.0 Hz, 1H), 7.06 (d, J=8.6, 2.0 Hz, 1H), 3.82-3.76 (m, 4H), 3.67-3.65 (m, 4H), 3.51-3.46 (m, 2H), 2.91 (t, J=7.6 Hz, 2H).

Step 4: N-[2-(5-Chloro-1H-indol-3-yl)ethyl]-2-morpholino-pyrimidine-5-carboxamide (130 g, 337.0 mmol) as mixed with dry acetonitrile (400 mL). Then POCl₃ (219 mL, 2360 mmol) was added in one portion. The mixture was stirred for 6 h at 90° C. The mixture was concentrated to give a residue. The residue was adjusted to PH=9-10. The mixture was extracted with ethyl acetate (3*200 mL) and filtered. The filtrate was concentrated to give a residue. The residue was washed with ethyl acetate (50 mL) to give 4-[5-(6-chloro-4,9-dihydro-3H-pyrido[3,4-b]indol-1-yl)pyrimidin-2-yl]morpholine (110 g, 88.76% yield) as a yellow solid. LCMS: ESI-MS: m/z: 368.0 [M+H]⁺, RT=1.850 min. ¹H NMR (400 MHz, DMSO-d₆) δ 11.53 (s, 1H), 8.75 (s, 2H), 7.71 (d, 1H, J=2.0 Hz), 7.44 (d, 1H, J=8.8 Hz), 7.22 (dd, 1H, J=8.8 Hz, 2.0 Hz), 3.88-3.81 (m, 6H), 3.70 (t, 4H, J=4.8 Hz), 2.86 (t, 2H, J=8.2 Hz).

Step 5: 4-[5-(6-Chloro-4,9-dihydro-3H-pyrido[3,4-b]indol-1-yl)pyrimidin-2-yl]morpholine (2.0 g, 5.4 mmol), RuCl[(R,R)-TsDPEN] (mesitylene) (34.0 mg, 0.0540 mmol) was dissolved in DMF (8.7 mL, 110 mmol), and formic acid triethylamine complex 5:2 (5.0 mL, 12 mmol) were added. The mixture was stirred at 25° C. for 18 h under N₂ protection and adjusted to PH=9-10 with Na₂CO₃ aqueous solution. The mixture was extracted with ethyl acetate (3*50 mL). The organic phase was washed with brine (4*30 mL), dried over anhydrous Na₂SO₄ and filtered. The filtrate was concentrated to give a residue. The residue was suspended in CH₂Cl₂/ethyl acetate (1:1 (v/v), 10 mL), filtered to collect filter cake and dried in vacuo to get 4-[5-[(1S)-6-chloro-2,3,4,9-tetrahydro-1H-pyrido[3,4-b]indol-1-yl]pyrimidin-2-yl]morpholine (1.2 g, 60% yield) as pale brown solid. LCMS: ESI-MS: m/z: 370.1 [M+H]⁻, RT=1.414 min.

¹H NMR (400 MHz, DMSO-d₆) δ 10.71 (s, 1H), 8.23 (s, 2H), 7.44 (d, J=2.0 Hz, 1H), 7.22 (d, J=8.4 Hz, 1H), 7.00 (dd, J=8.6, 2.0 Hz, 1H), 4.98 (s, 1H), 3.68-3.64 (m, 8H), 3.12-3.07 (m, 1H), 2.95-2.89 (m, 2H), 2.74-2.60 (m, 2H). Chiral HPLC: >99% ee value.

Step 6: To a 3-necked bottle, (S)-4-(5-(6-chloro-2,3,4,9-tetrahydro-1H-pyrido[3,4-b]indol-1-yl)pyrimidin-2-yl)morpholine (110 g, 297.5 mmol) was dissolved in CH₃CN (1130 mL). N,N-dimethylpyridin-4-amine (37.0 g, 299.8 mmol) was added slowly. Then 2-(trichloromethyl)-4,6-bis(trifluoromethyl)-1,3,5-triazine (120.0 g, 358.8 mmol) was added dropwise at 0° C. for 10 mins under N₂. After addition, reaction was stirred at rt for 6 h. LCMS showed about 50% conversion at 6 h and no 2-(trichloromethyl)-4,6-bis(trifluoromethyl)-1,3,5-triazine remained. Additional 2-(trichloromethyl)-4,6-bis(trifluoromethyl)-1,3,5-triazine (55 g, 164.5 mmol) was added dropwise to the heavily heterogeneous mixture, which was stirred at rt overnight. The mixture was concentrated to remove CH₃CN, then purified by column flash (eluent: gradient, from 100% petroleum ether to petroleum ether:ethyl acetate=80:20) to give Compound 406 (136 g, 78.18% yield) as a pale yellow solid. MS m/z 585.1 [M+H]⁺; ¹H NMR (400 MHz, DMSO) δ 11.12 (s, 1H), 8.34 (s, 2H), 7.58 (d, J=2.0 Hz, 1H), 7.34 (d, J=8.4 Hz, 1H), 7.11 (dd, J=8.8, 2.0 Hz, 1H), 6.86 (s, 1H), 4.97 (dd, J=9.6, 5.2 Hz, 1H), 3.70-3.61 (m, 8H), 3.58-3.51 (m, 1H), 3.06-3.01 (m, 1H), 2.90-2.82 (m, 1H).

### Example 17

### Preparation of Compound 498

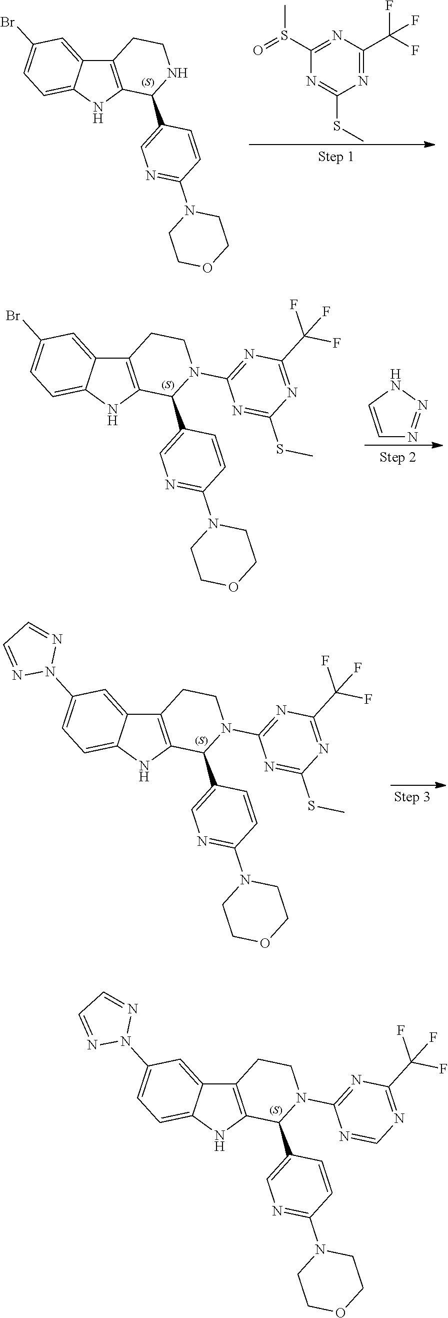

Step 1: A 8 mL screw-cap vial was charged with 4-[5-[(1S)-6-bromo-2,3,4,9-tetrahydro-1H-pyrido[3,4-b]indol-1-yl]-2-pyridyl]morpholine (104 mg, 0.25 mmol), 2-methylsulfanyl-4-methylsulfinyl-6-(trifluoromethyl)-1,3,5-triazine (80 mg, 0.31 mmol) and acetonitrile (2 mL), then 2,6-Lutidine (60 mg, 0.55 mol) was added and the resulting mixture was allowed to stir at ambient temperature for 3 h. The mixture partitioned between EtOAc and brine (1:1). The organic phase was washed with brine, dried over MgSO₄ and then purified by silca-gel chromatography, eluting with Hexanes:EtOAc (0-70% gradient) to afford 4-[5-[(1S)-6-bromo-2-[4-methylsulfanyl-6-(trifluoromethyl)-1,3,5-triazin-2-yl]-1,3,4,9-tetrahydropyrido[3,4-b]indol-1-yl]-2-pyridyl]morpholine (115 mg, 75.35% yield) was isolated as a white solid.

Step 2: An 8 mL screw-cap vial with a septum cap was charged with tris(dibenzylideneacetone)dipalladium(0) (10 mg, 0.01 mmol), 2-di-tert-butylphosphino-3,4′5,′-tetramethyl-2′,4′,6′-triisopropyl-1,1′-biphenyl (12 mg, 0.02 mmol) and toluene (1 mL), the mixture was degassed and backfilled with argon (3×), placed in a heating block pre-heated to 105° C. and allowed to stir for 2 min. The dark solution was transferred by syringe to another 8 mL screw-cap vial containing an argon sparged mixture of pre-active reaction of 4-[5-[(1S)-6-bromo-2-[4-methylsulfanyl-6-(trifluoromethyl)-1,3,5-triazin-2-yl]-1,3,4,9-tetrahydropyrido[3,4-b]indol-1-yl]-2-pyridyl]morpholine (115 mg, 0.19 mmol), potassium phosphate tribasic (100 mg, 0.46 mmol) suspended in toluene (1 mL) and 1,4-Dioxane (0.2 mL). The resulting mixture was allowed to stir at 100° C. for 8 h. After being allowed to cool to ambient temperature, the mixture was partitioned between EtOAc and water, the organic phase washed with brine, dried over MgSO₄, and evaporated to dryness under reduced pressure. The dark oily residue was subjected to silica gel chromatography, eluting with hexanes EtOAc (gradient 0-50%) to afford 4-[5-[(15)-2-[4-methylsulfanyl-6-(trifluoromethyl)-1,3,5-triazin-2-yl]-6-(triazol-2-yl)-1,3,4,9-tetrahydropyrido[3,4-b]indol-1-yl]-2-pyridyl]morpholine as an off-white solid.

Step 3: An 8 mL screw-cap vial equipped with a septum cap and a stir bar was charged with 4-[5-[(1S)-2-[4-methylsulfanyl-6-(trifluoromethyl)-1,3,5-triazin-2-yl]-6-(triazol-2-yl)-1,3,4,9-tetrahydropyrido[3,4-b]indol-1-yl]-2-pyridyl]morpholine (65 mg, 0.11 mmol), ethanol (1 mL) and Raney nickel (200 mg, 1.17 mmol) and was allowed to stir at 22° C. for 18 h. The resulting dark suspension was filtered and the filter cake rinsed with methanol. Volatiles were removed under reduced pressure and the dark residue obtained, purified by reversed phase chromatography, eluting with ACN/water (0.1% TFA modifier, 5-100% gradient), the desired fractions were combined and after lyophilization. Compound 498 (40 mg, 66.7% yield) was isolated as a white amorphous solid. MS m/z=549.7 [M+H]⁺; ¹H NMR (DMSO-d₆) δ: 11.22 (s, 1H), 11.17 (s, 1H), 8.90 (s, 1H), 8.85 (s, 1H), 8.02-8.09 (m, 5H), 7.99 (s, 4H), 7.71-7.78 (m, 2H), 7.44-7.53 (m, 2H), 7.39 (s, 2H), 6.99 (s, 1H), 6.77-6.91 (m, 4H), 4.93-5.03 (m, 1H), 4.81-4.92 (m, 1H), 3.57-3.63 (m, 11H), 3.29-3.44 (m, 13H), 2.94-3.05 (m, 2H), 2.76-2.94 (m, 3H). The NMR shows the presence of two rotamers in ˜1:1.2 ratio presumably due to the hindered rotation around the N—C bond connecting the triazine to the core.

Using the procedure described for Compound 498 above, additional compounds described herein below were prepared by substituting the appropriate starting materials, suitable reagents and reaction conditions, obtaining compounds such as those selected from:

| Cpd | Data |
|---|---|
| 454 | MS m/z 616.7 [M + H]⁺; ¹H NMR (DMSO-d₆, 400 MHz) δ 11.22 (s, 1H), 8.19 (s, |
| | 1H), 8.14 (s, 1H), 8.06 (s, 2H), 7.81 (dd, J = 2.0, 8.8 Hz, 1H), 7.54 (s, 1H), 7.4-7.5 |
| | (m, 2H), 6.93 (s, 1H), 6.82 (d, J = 8.8 Hz, 1H), 4.9-5.0 (m, 1H), 4.0-4.1 (m, 1H), |
| | 3.6-3.7 (m, 4H), 3.4-3.5 (m, 4H), 3.0-3.1 (m, 1H), 2.9-3.0 (m, 1H). |
| 455 | MS m/z = 630.5 [M + H]⁺; ¹HNMR (400 MHz, DMSO) δ 11.18 (s, 1H), 8.19 (d, |
| | J = 1.5 Hz, 1H), 8.07 (s, 1H), 7.81 (s, 1H), 7.72-7.79 (m, 1H), 7.54 (s, 1H), 7.48 |
| | (dd, J = 8.8, 2.0 Hz, 1H), 7.43 (d, J = 8.8 Hz, 1H), 6.92 (s, 1H), 6.74-6.88 (m, 1H), |
| | 4.90-5.04 (m, 1H), 3.66 (br t, J = 4.6 Hz, 4H), 3.45-3.53 (m, 1H), 3.42 (br t, J = 4.6 |
| | Hz, 4H), 3.01-3.13 (m, 1H), 2.83-2.99 (m, 1H), 2.37 (s, 3H) |

### Example 18

### Preparation of Compound 419

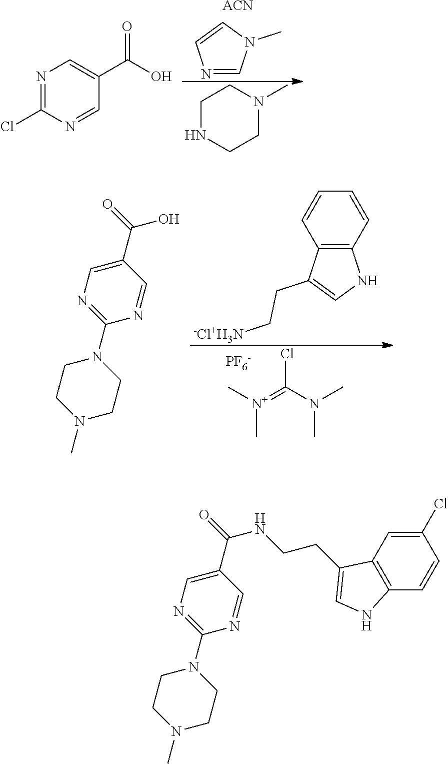

Step 1; To a 250-mL round-bottom flask equipped with a magnetic stir bar and a reflux condenser was added 2-chloropyrimidine-5-carboxylic acid (8.04 g, 49.2 mmol, 1.0 equiv). Acetonitrile (100 mL, 2 L/mol) was added at RT, followed by 1-methylimidazole (23 mL, 24 g, 289 mmol, 6.0 equiv) and 1-methylpiperazine (5.00 g, 48.9 mmol, 1.0 equiv). The reaction mixture was heated to reflux (90° C. heating block temperature) for 16 h, then cooled to RT. 5-Chlorotryptamine hydrochloride (11.8 g, 49.0 mmol, 1.0 equiv) was then added, followed by TCFH (21 g, 73.3 mmol, 1.5 equiv). An exotherm was observed, and the reaction reached a gentle reflux which eventually subsided after about 30 minutes. Measurement of a reaction aliquot by UPLC indicated essentially complete conversion at this point. After a further 30 minutes, the reaction mixture was concentrated on the rotavap to afford the crude product as an oil, which was split into 3 portions and purified on by silica gel chromatography (330 g silica each, gradient 0% to 100% MeOH in EtOAc. Product elutes at 50-60% MeOH). The resulting partially purified material was concentrated to afford a white paste, which was triturated with dichloromethane to afford N-[2-(5-chloro-1H-indol-3-yl)ethyl]-2-(4-methylpiperazin-1-yl)pyrimidine-5-carboxamide (15.72 g, 39.4 mmol, 81% yield) as a white powder, contaminated with <5% 1-methylimidazole. MS m/z 399 [M+H]^{+ 1}H NMR (400 MHz, DMSO-d₆) δ 11.07 (s, 1H), 8.85 (s, 2H), 8.65 (t, J=5.7 Hz, 1H), 7.59 (d, J=2.1 Hz, 1H), 7.35 (d, J=8.6 Hz, 1H), 7.26 (d, J=2.3 Hz, 1H), 7.05 (dd, J=8.6, 2.0 Hz, 1H), 4.78 (d, J=14.2 Hz, 2H), 3.59-3.34 (m, 6H), 3.12-2.98 (m, 2H), 2.92 (t, J=7.3 Hz, 2H), 2.78 (s, 1H).

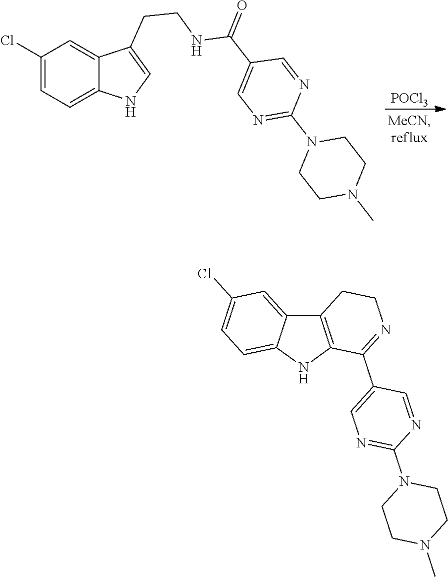 [SMILES: CN1CCN(c2ncc(C(=O)NCCc3c[nH]c4ccc(Cl)cc34)cn2)CC1 ; "POCl3" ; CN1CCN(c2ncc(C3=NCCc4c3[nH]c3ccc(Cl)cc43)cn2)CC1]

Step 2: N-[2-(5-chloro-1H-indol-3-yl)ethyl]-2-(4-methylpiperazin-1-yl)pyrimidine-5-carboxamide (5.00 g, 12.5 mmol, 1.00 equiv) was suspended in acetonitrile (50 mL, 4 L/mol) at RT in a 250-mL round-bottom flask equipped with a magnetic stir bar and a reflux condenser. Phosphoryl chloride (4.8 mL, 7.7 g, 52 mmol, 4 equiv) was added and the reaction mixture was heated to reflux. UPLC of reaction aliquot at 3 h indicated full conversion. The mixture was cooled to RT and acetonitrile and excess phosphorus oxychloride were removed on the rotavap. The resulting brown residue was cooled in an ice-water bath and cautiously dissolved in ca. 5 mL water (very exothermic). 50% aqueous NaOH (4 equiv) was added and the mixture was warmed to RT and stirred overnight. The resulting slurry was taken up in methanol and concentrated onto Celite. Purification by silica gel column chromatography (120 g silica, gradient 5% MeOH in EtOAc to 100% MeOH with 5% anhydrous ammonia) afforded 6-chloro-1-[2-(4-methylpiperazin-1-yl)pyrimidin-5-yl]-4,9-dihydro-3H-pyrido[3,4-b]indole (5.03 g, 12.5 mmol, 99%) as a yellow powder.

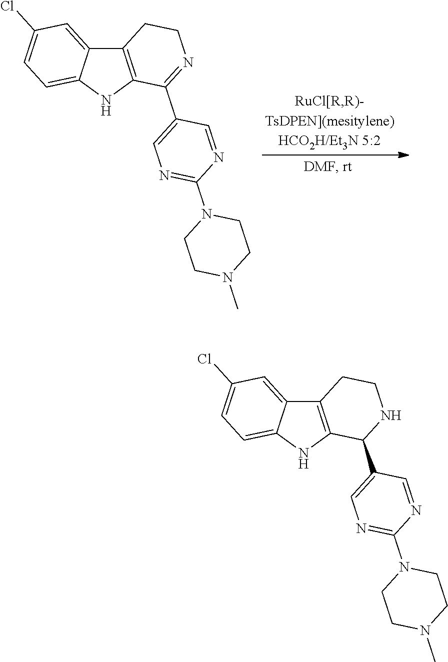 [SMILES: CN1CCN(c2ncc(C3=NCCc4c3[nH]c3ccc(Cl)cc43)cn2)CC1 ; CN1CCN(c2ncc([C@@H]3NCCc4c3[nH]c3ccc(Cl)cc43)cn2)CC1]

Step 3: RuCl[(R,R)-TsDPEN](mesitylene) (17 mg, 0.026 mmol, 0.010 equiv) was dissolved in formic acid triethylamine complex 5:2 (1.1 mL, 2.6 mmol, 1.0 equiv) at RT in a 20-mL vial equipped with a magnetic stir bar and a pressure-relief cap. 6-chloro-1-[2-(4-methylpiperazin-1-yl)pyrimidin-5-yl]-4,9-dihydro-3H-pyrido[3,4-b]indole (1.00 g, 2.49 mmol, 1.0 equiv) was added in one portion as the solid, followed by DMF (2.5 mL, 1 L/mol) which was used to rinse the walls of the vial. The septum was pierced with a needle to allow for venting of carbon dioxide that formed rapidly at reaction onset. After 3 h the reaction was found incomplete by UPLC of a reaction aliquot, and a further 17 mg of ruthenium catalyst was added. The reaction was deemed complete after an additional 1H. The resulting mixture was concentrated onto celite and purified by silica gel column chromatography (80 g silica, gradient 0% to 100%/MeOH in EtOAc) to afford (1S)-6-chloro-1-[2-(4-methylpiperazin-1-yl)pyrimidin-5-yl]-2,3,4,9-tetrahydro-1H-pyrido[3,4-b]indole (995 mg, 2.47 mmol, 99% yield) as a white solid.

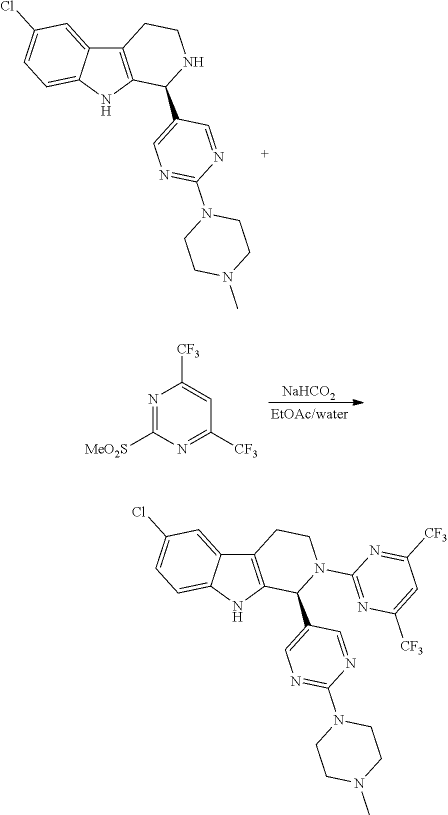

Step 4: To a 50-mL round bottom flask equipped with a magnetic stir bar was added (1S)-6-chloro-1-[2-(4-methylpiperazin-1-yl)pyrimidin-5-yl]-2,3,4,9-tetrahydro-1H-pyrido[3,4-b]indole (1.05 g, 2.61 mmol, 1.0 equiv), EtOAc (24 mL, 9 L/mol) and saturated aqueous NaHCO₃ (8 mL, 3 L/mol). 2-Methylsulfonyl-4,6-bis(trifluoromethyl)pyrimidine (1.55 g, 5.27 mmol, 2.0 equiv) was added in one portion and the resulting mixture was stirred at RT. UPLC analysis of as reaction aliquot indicated full conversion after 5 h. The reaction mixture was partitioned in a separatory funnel, and the aqueous layer was extracted with EtOAc (3×50 mL) and combined organic phases were washed with brine (50 mL), dried over sodium sulfate, filtered, and concentrated on the rotavap. Purification of the resulting crude oily residue by silica gel column chromatography (80 g silica, gradient 0% to 100% MeOH (containing 5% NH₃) in EtOAc) afforded Compound 419 (385 mg, 0.645 mmol, 25%) as a white solid. MS m/z 596.9 [M+H]^{+ 1}H NMR (400 MHz, DMSO-d₆) δ 11.07 (s, 1H), 8.41 (s, 2H), 7.66-7.49 (m, 2H), 7.36-7.30 (m, 1H), 7.10 (dd, J=8.6, 2.1 Hz, 1H), 6.79 (s, 1H), 4.97 (dd, J=13.6, 5.2 Hz, 1H), 4.68 (d, J=14.3 Hz, 2H), 3.49 (t, J=10.6 Hz, 3H), 3.20 (t, J=13.2 Hz, 2H), 3.01 (d, J=15.1 Hz, 3H), 2.92-2.76 (m, 4H).

Using the procedure described for Compound 419, above, an additional compound described herein was prepared by substituting the appropriate starting materials, suitable reagents and reaction conditions:

| Cpd | Data |
|---|---|
| 499 | MS m/z 642.8 [M + H]⁺; ¹H NMR (DMSO-d₆, 400 MHz) δ 11.09 (s, 1H), 8.32 (s, 2H), |
| | 7.66-7.75 (m, 1H), 7.55 (s, 1H), 7.29 (d, J = 8.5 Hz, 1H), 7.18-7.24 (m, 1H), 6.76 (s, 1H), |
| | 4.98 (br dd, J = 13.4, 4.9 Hz, 1H), 3.70 (br s, 4H), 3.44-3.57 (m, 1H), 2.94-3.06 (m, 1H), |
| | 2.76-2.92 (m, 1H), 2.25-2.37 (m, 4H), 2.19 (s, 3H) |

### Example 19

### Preparation of Compound 409

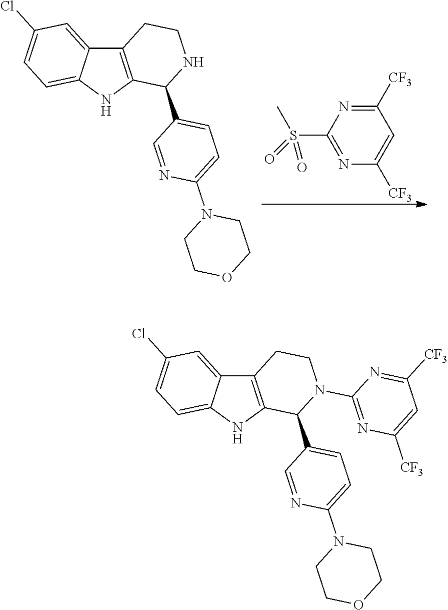 [SMILES: Clc1ccc2[nH]c3c(c2c1)CCN[C@H]3c1ccc(N2CCOCC2)nc1 ; CS(=O)(=O)c1nc(C(F)(F)F)cc(C(F)(F)F)n1 ; FC(F)(F)c1cc(C(F)(F)F)nc(N2CCc3c([nH]c4ccc(Cl)cc34)[C@@H]2c2ccc(N3CCOCC3)nc2)n1]

To a 50-mL round bottom flask equipped with a magnetic stir bar was added 4-[5-[(1S)-6-chloro-2,3,4,9-tetrahydro-1H-pyrido[3,4-b]indol-1-yl]-2-pyridyl]morpholine (2.2 g, 6.0 mmol), 2-methylsulfonyl-4,6-bis(trifluoromethyl)pyrimidine (3 g, 9.2 mmol) and Ethyl acetate (8 mL), the mixture was degassed and back filled with Argon. Then sodium bicarbonate (8 mL, 20 mmol saturated aqueous solution) was added at 0° C. under Argon. The mixture was stirred at RT overnight. Then it was diluted with EtOAc, phases separated, and the organic phase was washed with brine and dried over MgSO₄, then concentrated. The remaining oily residue was purified by silica gel chromatography, eluting with 0-30% EtOAc in hexane to afford Compound 409 (2.7 g, 78% yield). MS m/z 583.5 [M+H]⁺; ¹H NMR (DMSO-d₆, 400 MHz) δ: 11.13 (s, 1H), 8.15 (d, J=2.3 Hz, 1H), 7.56 (d, J=2.0 Hz, 1H), 7.53 (s, 1H), 7.45 (dd, J=2.4, 8.9 Hz, 1H), 7.31 (d, J=8.8 Hz, 1H), 7.09 (dd, J=2.1, 8.6 Hz, 1H), 6.89 (s, 1H), 6.81 (d, J=8.8 Hz, 1H), 4.9-5.0 (m, 1H), 3.6-3.7 (m, 4H), 3.47 (br d, J=4.0 Hz, 1H), 3.4-3.5 (m, 4H), 2.9-3.0 (m, 1H), 2.8-2.9 (m, 1H); mp: 148-152 C.

Using the procedure described for Compound 409, above, an additional compound described herein was prepared by substituting the appropriate starting materials, suitable reagents and reaction conditions:

| Cpd | Data |
|---|---|
| 410 | MS m/z = 583.8 [M + H]⁺; ¹H NMR (DMSO-d₆, 400 MHz) δ: 11.08 (s, 1H), 8.35 (s, |
| | 2H), 7.56 (d, J = 10.3 Hz, 2H), 7.33 (d, J = 8.8 Hz, 1H), 7.0-7.2 (m, 1H), 6.77 (s, |
| | 1H), 4.98 (br dd, J = 4.9, 13.4 Hz, 1H), 3.64 (br dd, J = 4.4, 17.6 Hz, 8H), 3.52 (br s, |
| | 1H), 2.8-3.1 (m, 2H). |
| 382 | MS m/z = 463.0 [M − H]⁻; ¹HNMR (400 MHz, DMSO) δ 11.13 (br s, 1H), 8.64-8.69 (m, |
| | 1H), 8.56-8.63 (m, 1H), 8.31-8.42 (m, 2H), 7.52-7.61 (m, 1H), 7.27-7.38 (m, 1H), |
| | 7.04-7.15 (m, 1H), 6.84-6.94 (m, 1H), 4.66 (br d, J = 14.0 Hz, 1H), 3.58-3.74 (m, 8H), |
| | 3.47-3.58 (m, 1H), 3.25-3.35 (m, 1H), 2.81-3.04 (m, 1H), 1.05-1.42 (m, 1H) |
| 462 | MS m/z = 516.0 [M + H]⁺; ¹H NMR (DMSO-d, 400 MHz) δ 11.21 (d, J = 20.8 Hz, 1H), |
| | 8.93 (d, J = 25.2 Hz, 1H), 8.29 (s, 1H), 8.13 (d, J = 2.0 Hz, 1H), 7.56 (d, J = 2.0 Hz, 1H), |
| | 7.47-7.43 (m, 1H), 7.33 (q, J = 4.0 Hz, 1H), 7.10 (t, J = 7.6 Hz, 1H), 7.06 (d, J = 50.8 Hz, |
| | 1H), 6.82 (q, J = 3.6 Hz, 1H), 5.03-4.87 (m, 1H), 3.66 (t, J = 4.0 Hz, 4H), 3.42 (t, J = 4.4 |
| | Hz, 4H), 3.36 (t, J = 8.8 Hz, 1H), 2.99 (q, J = 12.0 Hz, 1H), 2.85 (q, J = 11.2 Hz, 1H). |

### Example 20

### Preparation of Compound 17

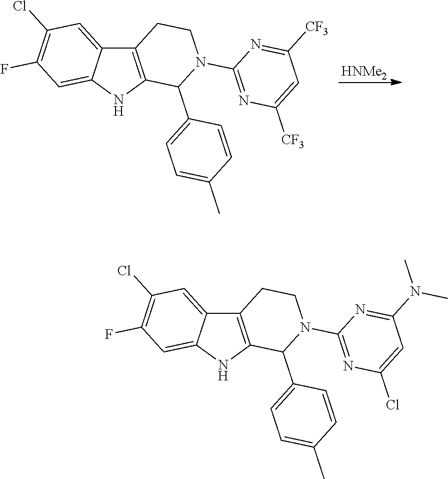 [SMILES: Cc1ccc(C2c3[nH]c4cc(F)c(Cl)cc4c3CCN2c2nc(C(F)(F)F)cc(C(F)(F)F)n2)cc1 ; Cc1ccc(C2c3[nH]c4cc(F)c(Cl)cc4c3CCN2c2nc(Cl)cc(N(C)C)n2)cc1 ; "HNMe2"]

The crude 6-chloro-2-(4,6-dichloropyrimidin-2-yl)-7-fluoro-1-(p-tolyl)-1,3,4,9-tetrahydropyrido[3,4-b]indole was prepared as the mother liquor in the preparation of 6-chloro-2-(4,6-dichloropyrimidin-2-yl)-7-fluoro-1-(p-tolyl)-1,3,4,9-tetrahydropyrido[3,4-b]indole starting with 137 mg of 4,6-dichloro-N-[2-(5-chloro-6-fluoro-1H-indol-3-yl)ethyl]pyrimidin-2-amine, as described in Example 4. The material was concentrated and dissolved in ˜1 mL of ethanol. To a microwave vial with a septum and stirbar was added this solution in ethanol, Me₂NH (7.9 mol/L) in water (0.5 mL, 4 mmol, 7.9 mol/L, 10), and heated to 100 C for 10 min. The mixture was concentrated, partitioned between water and DCM, purified via flash chromatography (silica 12+5 g): elute with EtOAc/Hexane gradient: 9% to 30%. The collected fractions were concentrated, dissolved in ethanol, precipitated with water, filtered, washed with water, and dried to provide Compound 017 as a white solid material (0.064 g, 0.14 mmol, 36% from 137 mg of 4,6-dichloro-N-[2-(5-chloro-6-fluoro-1H-indol-3-yl)ethyl]pyrimidin-2-amine). MS m/z 470.2 [M+H]⁺; ¹H NMR (DMSO-d₆) δ:11.23 (br s, 1H), 7.64 (d, J=7.3 Hz, 1H), 7.30 (br d, J=10.1 Hz, 1H), 7.11-7.22 (m, 4H), 7.02 (br s, 1H), 6.05 (s, 1H), 4.78 (br s, 1H), 3.06 (br s, 7H), 2.77 (br s, 2H), 2.27 (s, 3H).

Using the procedure described for Compound 17 above, additional compounds described herein were prepared by substituting the appropriate starting materials, suitable reagents and reaction conditions, obtaining compounds such as those selected from:

| Cpd | Data |
|---|---|
| 290 | MS m/z = 506.0 [M + H]⁺; ¹H NMR (500 MHz, DMSO-d₆) δ 11.07 (d, J = 16.6 Hz, |
| | 1H), 8.32 (d, J = 10.7 Hz, 2H), 7.55 (d, J = 2.1 Hz, 1H), 7.32 (dd, J = 8.6, 2.6 Hz, |
| | 1H), 7.09 (dd, J = 8.5, 2.1 Hz, 1H), 6.81 (d, J = 83.0 Hz, 1H), 4.92 (ddd, J = 85.3, |
| | 13.2, 5.3 Hz, 1H), 3.78-3.55 (m, 8H), 3.46-3.33 (m, 1H), 2.95 (dt, J = 14.8, |
| | 4.1 Hz, 1H), 2.86-2.70 (m, 1H), 2.36 (d, J = 33.9 Hz, 3H), 1.95 (s, 6H). |
| 295 | MS m/z = 493.0 [M + H]⁺; ¹H NMR (500 MHz, DMSO-d₆) δ 11.02 (d, J = 8.2 Hz, |
| | 1H), 8.26 (d, J = 10.1 Hz, 2H), 7.47 (d, J = 2.3 Hz, 1H), 7.25 (dd, J = 8.7, 1.8 Hz, |
| | 1H), 7.02 (dd, J = 8.6, 2.1 Hz, 1H), 6.82 (d, J = 54.9 Hz, 1H), 4.93 (ddd, J = 61.4, |
| | 13.4, 5.4 Hz, 1H), 3.84 (d, J = 34.2 Hz, 3H), 3.70-3.49 (m, 8H), 3.25-3.12 (m, |
| | 1H), 2.85 (dd, J = 15.3, 3.8 Hz, 1H), 2.70 (ddd, J = 20.9, 12.5, 6.0 Hz, 1H), 2.28 |
| | (d, J = 34.3 Hz, 3H). |
| 296 | MS m/z = 548.0 [M + H]⁺; ¹H NMR (500 MHz, DMSO-d₆) δ 11.03 (d, J = 37.3 Hz, |
| | 1H), 8.29 (d, J = 3.6 Hz, 2H), 7.52 (s, 1H), 7.31 (d, J = 8.6 Hz, 1H), 7.07 (dd, J = |
| | 8.6, 2.1 Hz, 1H), 6.91 (d, J = 29.7 Hz, 1H), 4.99 (ddd, J = 53.7, 13.4, 5.3 Hz, 1H), |
| | 3.83-3.53 (m, 16H), 3.30-3.04 (m, 1H), 2.86 (dd, J = 15.5, 3.9 Hz, 1H), 2.72 (d, |
| | J = 12.9 Hz, 1H), 2.24 (d, J = 32.7 Hz, 3H). |
| 297 | MS m/z = 492.0 [M + H]⁺; ¹H NMR (500 MHz, DMSO-d₆) δ 11.06 (d, J = 25.8 Hz, |
| | 1H), 8.53-8.06 (m, 3H), 7.53 (d, J = 2.1 Hz, 1H), 7.31 (d, J = 8.6 Hz, 1H), 7.07 |
| | (dd, J = 8.7, 2.1 Hz, 1H), 6.88 (s, 1H), 5.00 (d, J = 16.5 Hz, 1H), 3.79-3.52 (m, |
| | 8H), 3.29-2.99 (m, 7H), 2.87 (dd, J = 15.2, 3.8 Hz, 1H), 2.74 (s, 1H). |
| 315 | MS m/z = 590.0 [M + H]⁺; ¹H NMR (500 MHz, DMSO-d₆) δ 11.09 (d, J = 15.5 Hz, |
| | 1H), 8.29 (s, 2H), 7.52 (s, 1H), 7.30 (dd, J = 8.7, 2.8 Hz, 1H), 7.07 (d, J = 8.7 Hz, |
| | 1H), 6.87 (d, J = 66.2 Hz, 1H), 4.97 (dd, J = 67.1, 12.5 Hz, 1H), 4.09-3.03 (m, |
| | 15H), 2.78 (dd, J = 56.0, 11.5 Hz, 2H), 2.21 (d, J = 27.9 Hz, 3H), 1.83-1.10 (m, |
| | 8H), 1.08-0.54 (m, 6H). |
| 317 | MS m/z = 590.0 [M + H]⁺; ¹H NMR (400 MHz, DMSO-d₆) δ 11.06 (d, J = 14.8 Hz, |
| | 1H), 8.50-8.10 (m, 2H), 7.52 (s, 1H), 7.30 (d, J = 8.6 Hz, 1H), 7.07 (dd, J = 8.6, |
| | 2.1 Hz, 1H), 7.00-6.71 (m, 1H), 5.35-4.76 (m, 1H), 3.86-3.43 (m, 10H), 3.10 |
| | (d, J = 30.8 Hz, 4H), 2.95-2.63 (m, 2H), 2.22 (d, J = 23.2 Hz, 3H), 1.52 (s, 2H), |
| | 1.28 (dd, J = 22.1, 13.8 Hz, 3H), 0.91 (q, J = 8.2, 7.2 Hz, 3H). |
| 318 | MS m/z = 535.0 [M + H]⁺; ¹H NMR (400 MHz, DMSO-d₆) δ 11.09 (d, J = 8.7 Hz, |
| | 1H), 8.32 (s, 2H), 7.55 (s, 1H), 7.33 (d, J = 8.6 Hz, 1H), 7.09 (dd, J = 8.6, 2.1 Hz, |
| | 1H), 6.89 (d, J = 43.9 Hz, 1H), 5.33-4.68 (m, 1H), 4.33 (dt, J = 28.6, 6.3 Hz, 2H), |
| | 3.77-3.53 (m, 8H), 2.91 (d, J = 15.1 Hz, 1H), 2.78 (d, J = 13.3 Hz, 1H), 2.34 (d, |
| | J = 26.9 Hz, 3H), 1.69 (dt, J = 14.2, 7.2 Hz, 2H), 1.41 (p, J = 7.6 Hz, 2H), 0.94 (q, |
| | J = 7.3 Hz, 3H). |
| 456 | MS m/z = 540.0 [M + H]⁺; ¹H NMR (400 MHz, DMSO-d₆) δ 11.06 (s, 1H), 8.30 (s, |
| | 2H), 8.04 (d, J = 5.6 Hz, 1H), 7.51 (d, J = 2.1 Hz, 1H), 7.29 (d, J = 8.6 Hz, 1H), |
| | 7.06 (dd, J = 8.6, 2.1 Hz, 1H), 6.86 (s, 1H), 5.88 (d, J = 5.6 Hz, 1H), 4.98 (d, J = |
| | 12.5 Hz, 1H), 4.64-4.33 (m, 4H), 3.77-3.55 (m, 8H), 3.20 (t, J = 11.7 Hz, 1H), |
| | 2.98-2.62 (m, 2H). ¹⁹F NMR (376 MHz, DMSO-d₆) δ −98.99 (p, J = 12.4 Hz). |
| 457 | MS m/z = 554.0 [M + H]⁺; ¹H NMR (400 MHz, DMSO-d₆) δ 11.08 (d, J = 5.0 Hz, |
| | 1H), 8.31 (d, J = 12.7 Hz, 2H), 7.53 (d, J = 2.3 Hz, 1H), 7.30 (t, J = 8.0 Hz, 1H), |
| | 7.07 (dd, J = 8.7, 2.1 Hz, 1H), 6.87 (d, J = 53.9 Hz, 1H), 5.21-4.84 (m, 1H), 4.66- |
| | 4.32 (m, 3H), 3.64 (dd, J = 15.6, 4.9 Hz, 8H), 3.32-3.20 (m, 2H), 2.97-2.82 |
| | (m, 1H), 2.83-2.61 (m, 1H), 2.26 (d, J = 28.6 Hz, 3H). ¹⁹F NMR (376 MHz, |
| | DMSO-d₆) δ −99.52. |

### Example 21

### Preparation of Compound 291

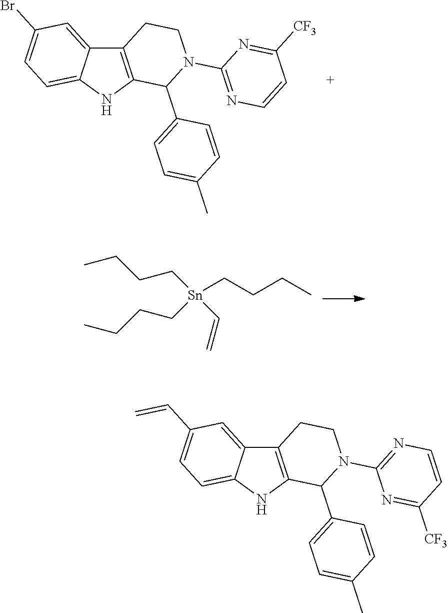 [SMILES: Cc1ccc(C2c3[nH]c4ccc(Br)cc4c3CCN2c2nccc(C(F)(F)F)n2)cc1 ; C=[CH][Sn]([CH2]CCC)([CH2]CCC)[CH2]CCC ; C=Cc1ccc2[nH]c3c(c2c1)CCN(c1nccc(C(F)(F)F)n1)C3c1ccc(C)cc1]

To a screw-cap vial with a septum and a needle for nitrogen was added: 6-bromo-1-(p-tolyl)-2-[4-(trifluoromethyl)pyrimidin-2-yl]-1,3,4,9-tetrahydropyrido[3,4-b]indole (0.357 g, 0.733 mmol), [1,1′-bis(diphenylphophini)ferrocene]dichloropalladium(II) (0.029 g, 0.038 mmol). The vial was flushed with nitrogen and toluene (6 mL), tributyl(vinyl)tin (0.3 mL, 1 mmol), were added in succession. The mixture was heated to 110° C. (heating block) for 1.5 h, then cooled to RT and loaded directly onto a silica gel column. Purified via flash chromatography, eluting with hexane/EtOAc gradient:10% to 50% to provide white solid, Compound 297 (0.261 g, 82% yield). MS m/z 435.2 [M+H]⁺; ¹H NMR (DMSO-H₆) δ: 11.10 (br s, 1H), 8.84 (br s, 1H), 7.60 (s, 1H), 7.31-7.37 (m, 2H), 7.25-7.31 (m, 2H), 7.20-7.25 (m, 2H), 7.15 (d, J=4.9 Hz, 1H), 7.04-7.14 (m, 1H), 6.87 (dd, J=17.5, 10.8 Hz, 1H), 5.77 (d, J=17.7 Hz, 1H), 5.16 (d, J=11.0 Hz, 1H), 4.79-5.09 (m, 1H), 3.29-3.38 (m, 1H), 2.84-3.03 (m, 2H), 2.34 (s, 3H).

### Example 22

### Preparation of Compound 292

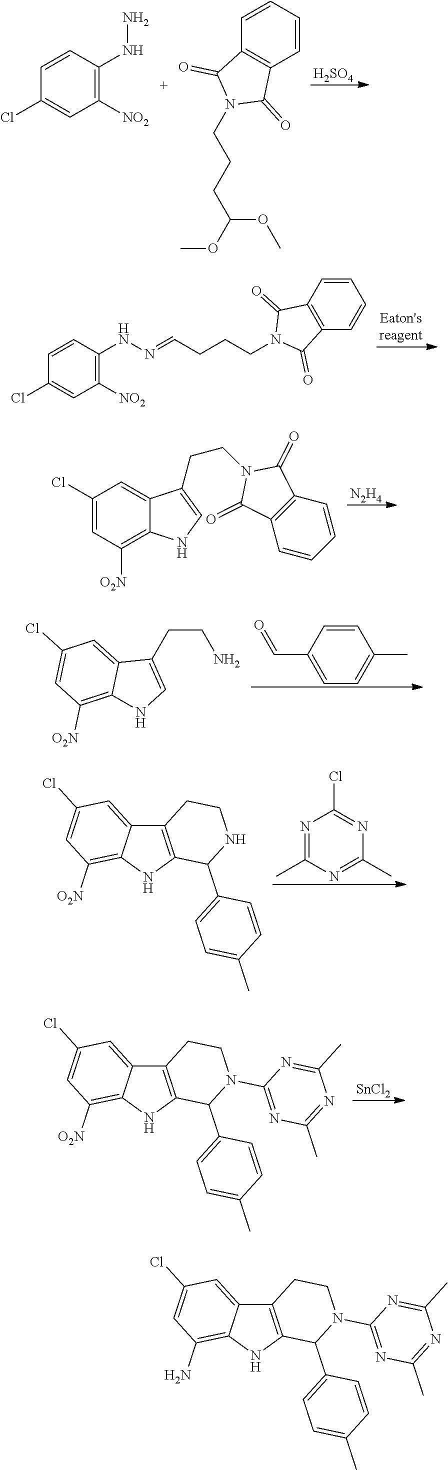

Step 1. To a 1-neck RBF was added: (4-chloro-2-nitrophenyl)hydrazine (1.77 g, 9.25 mmol), 1H-isoindole-1,3(2H)-dione, 2-(4,4-dimethoxybutyl)-(2.57 g, 9.27 mmol), ethanol (30 mL), followed by 4N sulfuric acid (2.5 mL, 5.0 mmol). Warmed to 50° C. for 1H. A thick suspension formed. The reaction mixture was diluted with water, filtered, washed with EtOH/water ˜2/1, then water and dried to provide orange solid, 2-[(4E)-4-[(4-chloro-2-nitro-phenyl)hydrazono]butyl]isoindoline-1,3-dione (3.50 g, 9.05 mmol, 98% yield). MS m/z 385.1 [M−H]⁻.

Step 2: To a screw-cap vial with a septum and a needle for nitrogen was added: N—[(E)-butylideneamino]-4-chloro-2-nitro-aniline (0.57 g, 1.5 mmol), sulfolane (6 mL), Eaton's reagent (3 mL). The mixture was heated to 80 C for 1.5 h, then added another 3 mL of Eaton's reagent and heated to 100° C. for 40 min. The reaction mixture was cooled to RT diluted with water, filtered, washed w water, ether, and dried to provide 0.8 g of brown solid used in the next step. MS m/z 367.8 [M−H]⁻.

Step 3: To a 1-neck RBF, equipped with a septum and N2 inlet, added 2-[2-(5-chloro-7-nitro-1H-indol-3-yl)ethyl]isoindoline-1,3-dione from prep step, ethanol (50 mL), hydrazine hydrate (0.5 mL, 10 mmol). The mixture was heated to 80° C. for 1.5 h, then added 2 mL aq. N₂H₄. After heating for another ½ h, the mixture was concentrated, diluted with water, partially concentrated, filtered, washed with water and dried to provide brownish orange solid, 2-(5-chloro-7-nitro-1H-indol-3-yl)ethanamine (E, 0.216 g, 0.901 mmol, 61% yield), ˜80% pure by HPLC. MS m/z 240.0 [M+H]⁺.

Step 4: To a screw-cap vial with a septum and a needle for nitrogen was added 2-(5-chloro-7-nitro-1H-indol-3-yl)ethanamine (0.216 g, 0.901 mmol), p-tolualdehyde (0.176 g, 1.46 mmol), acetic acid (6 mL), methanesulfonic acid (0.1 mL, 2 mmol) and the mixture was heated to 110° C. for 5 h. Then additional 0.25 mL of aldehyde was added and the mixture was heated to 140° C. for 2 days. The mixture was cooled, diluted with aq. NaHCO₃, extracted with DCM, conc, purified by chromatography using a gradient DCM/MeOH (with 2% NH₄OH) from 1% to 100% to provide brown solid material, ˜70% pure by UPLC, 6-chloro-8-nitro-1-(p-tolyl)-2,3,4,9-tetrahydro-1H-pyrido[3,4-b]indole (0.129 g, 0.377 mmol). MS m/z 342.1 [M+H]⁺.

Step 5: To a microwave vial with a septum and stirbar was added 6-chloro-8-nitro-1-(p-tolyl)-2,3,4,9-tetrahydro-1H-pyrido[3,4-b]indole (0.129 g, 0.377 mmol), 2-chloro-4,6-dimethyl-1,3,5-triazine (0.053 g, 0.36 mmol), 1,4-dioxane (3 mL), triethylamine (0.15 mL, 1.1 mmol) and the vial was heated in a microwave oven to 180° C. for 30 min. The material was purified via flash chromatography eluted with DCM/EtOAc gradient:5% to 100% to provide dark orange glassy solid material, 6-chloro-2-(4,6-dimethyl-1,3,5-triazin-2-yl)-8-nitro-1-(p-tolyl)-1,3,4,9-tetrahydropyrido[3,4-b]indole (0.118 g, 0.263 mmol, 73% yield). MS m/z 449.6 [M+H]⁺.

Step 6: To a screw-cap vial with a septum and a needle for nitrogen was added 6-chloro-2-(4,6-dimethyl-1,3,5-triazin-2-yl)-8-nitro-1-(p-tolyl)-1,3,4,9-tetrahydropyrido[3,4-b]indole (0.114 g, 0.254 mmol), dichlorotin dihydrate (0.130 g, 0.576 mmol), ethanol (5 mL), and the mixture was heated to 70° C. for 2 h. Then added 0.27 g SnCl₂×2H₂O and continued at 70° C. 2 h. The mixture was partitioned between aq. NaHCO₃ and DCM, conc, and purified via flash chromatography eluted with hexane/EtOAc gradient 20% to 100%. The isolated material was dissolved in ether, precipitated with hexane, partially concentrated to provide white solid material, Compound 292 (0.050 g, 0.12 mmol, 47% yield). MS m/z 419.2 [M+H]⁺. ¹H NMR (DMSO-d₆) δ:10.65 (s, 1H), 7.21-7.26 (m, 2H), 7.16-7.21 (m, 2H), 7.12 (s, 1H), 6.73 (d, J=1.5 Hz, 1H), 6.34 (d, J=1.5 Hz, 1H), 5.24 (br s, 1H), 4.92 (dd, J=13.1, 4.9 Hz, 1H), 3.11-3.21 (m, 1H), 2.67-2.84 (m, 2H), 2.40 (s, 3H), 2.33 (s, 3H), 2.29 (s, 3H).

Using the procedure described for Compound 292 above, additional compounds described herein were prepared by substituting the appropriate starting materials, suitable reagents and reaction conditions, obtaining compounds such as those selected from:

| Cpd | Data |
|---|---|
| 309 | MS m/z 492.1 [M + H]⁺; ¹H NMR (DMSO-d₆) δ: 10.52 (s, 1H), |
| | 8.38 (s, 2H), 6.87 (s, 1H), 6.75 (d, J = 0.9 Hz, 1H), 6.36 (d, J = |
| | 1.2 Hz, 1H), 5.24 (br s, 2H), 5.04 (dd, J = 13.4, 5.2 Hz, 1H), |
| | 3.58-3.73 (m, 8H), 3.19-3.44 (m, 1H), 2.79-2.88 (m, 1H), 2.63- |
| | 2.75 (m, 1H), 2.41 (s, 3H), 2.34 (s, 3H). Used Fe/HCl for the last |
| | step. |
| 310 | MS m/z 531.6 [M + H]⁺; ¹H NMR (DMSO-d₆) δ: 10.51 (s, 1H), |
| | 8.79 (d, J = 4.6 Hz, 1H), 8.39 (s, 2H), 7.11 (d, J = 4.9 Hz, 1H), |
| | 6.76 (s, 2H), 6.36 (s, 1H), 5.23 (br s, 2H), 4.95-5.08 (m, 1H), |
| | 3.57-3.73 (m, 8H), 3.38-3.47 (m, 1H), 2.80-2.89 (m, 1H), 2.68- |
| | 2.80 (m, 1H). Used Fe/HCl for the last step. |

### Example 23

### Preparation of Compound 362

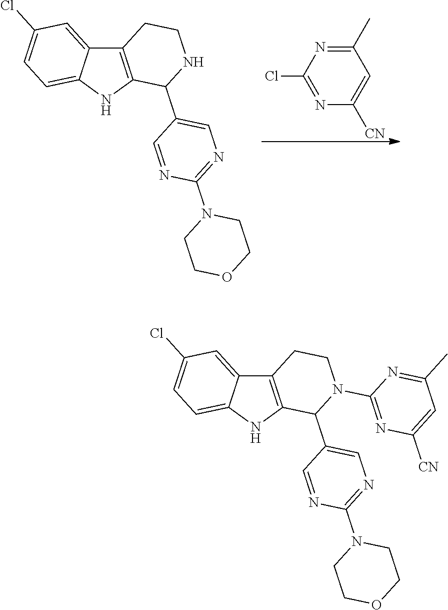

To a 50 mL single neck round-bottomed flask 4-[5-(6-chloro-2,3,4,9-tetrahydro-1H-pyrido[3,4-b]indol-1-yl)pyrimidin-2-yl]morpholine (100 mg, 0.2704 mmol) and 2-chloro-6-methyl-pyrimidine-4-carbonitrile (70 mg, 0.45582 mmol) were dissolved in 1-butanol (5.0 mL, 55 mmol) then N,N-diisopropylethylamine (0.3 mL, 2 mmol) was added into the mixture. The reaction mixture was stirred at 120° C. for 12 Hours. The resulting mixture was concentrated under vacuum and purified by Prep-HPLC to get Compound 362 (C, 110 mg, 0.2259 mmol, 83.53% yield) as light yellow solid.

MS m/z=486.9, [M+H]; ¹H NMR (400 MHz, DMSO) δ 11.07 (s, 1H), 8.32 (s, 2H), 7.54 (d, J=2.0 Hz, 1H), 7.31 (d, J=8.8 Hz, 1H), 7.17 (s, 1H), 7.08 (q, J=2.0 Hz, 1H), 6.81 (s, 1H), 4.94 (s, 1H), 3.66 (d, J=4.0 Hz, 4H), 3.61 (d, J=3.4 Hz, 4H) 3.30-3.31 (m, 1H), 2.90 (d, J=3.2 Hz, 1H), 2.73-2.81 (m, 1H), 2.43 (s, 3H).

Using the procedure described for Compound 362 above, additional compounds described herein were prepared by substituting the appropriate starting materials, suitable reagents, and reaction conditions, obtaining compounds such as those selected from:

| Cpd | Data |
|---|---|
| 363 | MS m/z = 436.0, [M + H]⁺; ¹H NMR (400 MHz, DMSO) δ 11.14 |
| | (s, 1H), 8.75 (d, J = 4.8 Hz, 1H), 7.53 (d, J = 2.0 Hz, 1H), 7.32 |
| | (d, J = 8.8 Hz, 1H), 7.28-7.24 (m, 2H), 7.08 (dd, J = 8.8, 2.0 Hz, |
| | 1H), 7.04 (t, J = 7.4 Hz, 1H), 6.73 (t, J = 7.0 Hz, 1H), 4.85-4.82 |
| | (m, 1H), 3.38-3.31 (m, 1H), 2.90-2.79 (m, 2H), 2.26 (d, J = |
| | 1.6 Hz, 3H). |

### Example 24

### Preparation of Compound 369

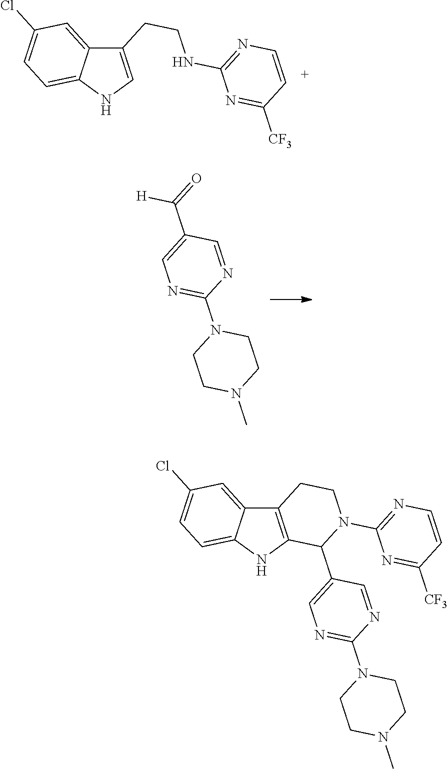 [SMILES: FC(F)(F)c1ccnc(NCCc2c[nH]c3ccc(Cl)cc23)n1 ; CN1CCN(c2ncc(C=O)cn2)CC1 ; CN1CCN(c2ncc(C3c4[nH]c5ccc(Cl)cc5c4CCN3c3nccc(C(F)(F)F)n3)cn2)CC1]

To a 50 mL round-bottom flask was added, N-[2-(5-chloro-1H-indol-3-yl)ethyl]-4-(trifluoromethyl)pyrimidin-2-amine (150 mg, 0.4403 mmol), 2-(4-methylpiperazin-1-yl)pyrimidine-5-carbaldehyde (112 mg, 0.54306 mmol) p-toluenesulfonic acid (45 mg, 0.26132 mmol) in 2-butanol (4 mL, 43.5 mmol) The mixture was stirred at 100° C. under nitrogen atmosphere for 6 h. LCMS showed near complete conversion to the desired product. Purified by flash and Prep-HPLC to get Compound 369 (80 mg, 0.1512 mmol, 34.35% yield) as white solid. MS m/z=529.0, [M+H]; ¹H NMR (400 MHz, DMSO) δ 11.10 (s, 1H), 8.79 (d, J=4.8 Hz, 1H), 8.32 (s, 2H), 7.55 (d, J=2 Hz, 1H), 7.32 (d, J=8.4 Hz, 1H), 7.12 (d, J=4.8 Hz, 1H), 7.09 (q, J=6.4 Hz, 1H), 6.80 (s, 1H), 5.00 (t, J=2.8 Hz, 1H), 3.70 (s, 4H), 3.43 (t, J=3.2 Hz, 1H), 2.94 (q, J=12.4 Hz, 1H), 2.81 (m, 1H), 2.34 (s, 4H), 2.21 (s, 3H).

Using the procedure described for Compound 369, above, an additional compound described herein was prepared by substituting the appropriate starting materials, suitable reagents, and reaction conditions, obtaining compounds such as those selected from:

| Cpd | Data |
|---|---|
| 371 | MS m/z = 447.0, [M + H]⁺; (DMSO-d, 400 MHz) 11.20 (s, 1H), |
| | 8.79 (d, J = 4.4 Hz, 1H), 7.54 (s, 1H), 7.43-7.36 (m, 2H), 7.32 (d, |
| | J = 8.4 Hz, 1H), 7.25 (t, J = 9.6 Hz, 1H), 7.14 (q, J = 3.6 Hz, 2H), |
| | 7.08 (q, J = 6.8 Hz, 1H), 7.01 (t, J = 8.0 Hz, 1H), 4.86 (s, 1H), |
| | 3.32 (d, J = 5.2 Hz, 1H), 2.90-2.83 (m, 2H). |

### Example 25

### Preparation of Compound 370

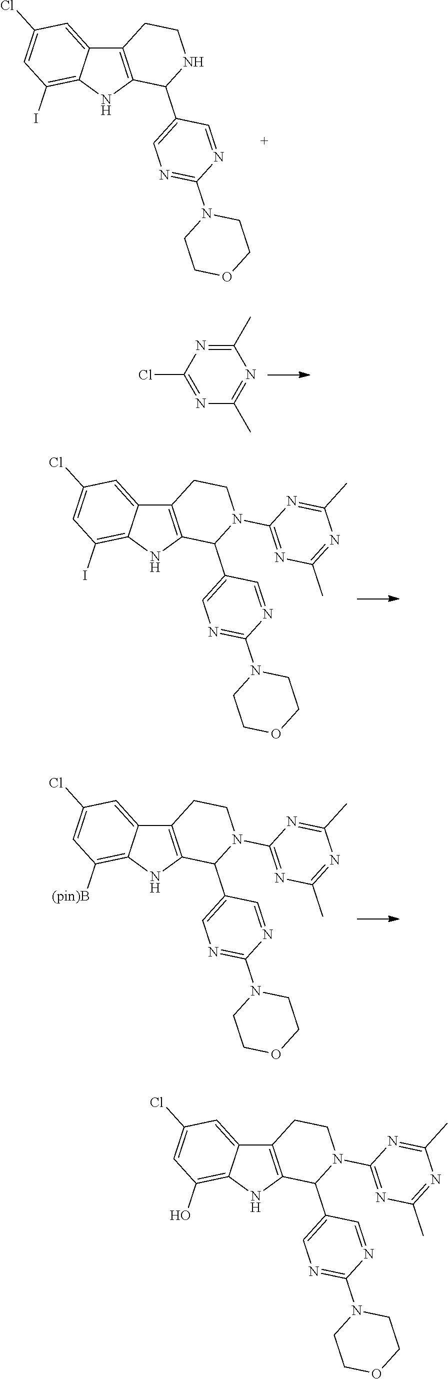

Step 1: To a 1-neck RBF, equipped with an N₂ inlet, were added: 4-[5-(6-chloro-8-iodo-2,3,4,9-tetrahydro-1H-carbazol-1-yl)pyrimidin-2-yl]morpholine (300 mg, 0.6063 mmol), 2-chloro-4,6-dimethyl-1,3,5-triazine (130 mg, 0.90548 mmol), and THF (5 mL, 61.4 mmol), followed by N,N-diisopropylamine (0.7 mL, 4 mmol). The reaction mixture was heated to 70° C. for 7 h, concentrated to dryness, and purified by column flash to afford 4-[5-[6-chloro-2-(4,6-dimethyl-1,3,5-triazin-2-yl)-8-iodo-1,3,4,9-tetrahydropyrido[3,4-b]indol-1-yl]pyrimidin-2-yl]morpholine as a pale brown solid. (200 mg, 0.3317 mmol, 54.71% yield) MS m/z 603.1 [M+H]⁺.

Step 2: To a solution of 4-[5-[6-chloro-2-(4,6-dimethyl-1,3,5-triazin-2-yl)-8-iodo-1,3,4,9-tetrahydropyrido[3,4-b]indol-1-yl]pyrimidin-2-yl]morpholine (200.0 mg, 0.3317 mmol) in 1,4-dioxane (4 mL, 46.85 mmol) was added 4,4,5,5-tetramethyl-2-(4,4,5,5-tetramethyl-1,3,2-dioxaborolan-2-yl)-1,3,2-dioxaborolane (140.0 mg, 0.5514 mmol), potassium acetate (80.0 mg, 0.815 mmol) and 1,1′-bis(diphenylphosphino)ferrocene]dichloropalladium(II) (30.0 mg, 0.0389 mmol), then the mixture was evacuated, refilled with N₂ and heated to 100° C. for 16 h. The reaction mixture was concentrated and purified via column flash and prep-HPLC to provide 4-[5-[6-chloro-2-(4,6-dimethyl-1,3,5-triazin-2-yl)-8-(4,4,5,5-tetramethyl-1,3,2-dioxaborolan-2-yl)-1,3,4,9-tetrahydropyrido[3,4-b]indol-1-yl]pyrimidin-2-yl]morpholine (C, 50 mg, 0.08293 mmol, 25.00% yield) MS m/z 603.6 [M+H]⁺.

Step 3: To a solution of 4-[5-[6-chloro-2-(4,6-dimethyl-1,3,5-triazin-2-yl)-8-(4,4,5,5-tetramethyl-1,3,2-dioxaborolan-2-yl)-1,3,4,9-tetrahydropyrido[3,4-b]indol-1-yl]pyrimidin-2-yl]morpholine (50.0 mg, 0.0829 mmol) in THF (2 mL) was added hydrogen peroxide in water (0.4 mL, 35 mass %) and sodium hydroxide (0.4 mL, 0.4 mmol, 1 mol/L). The mixture was stirred for 30 min, concentrated in vacuo, purified by prep-TLC to get crude product. Further purification by prep-HPLC yielded Compound 370 (35 mg, 0.07099 mmol, 85.6% yield) as white solid.

MS: m/z=493.0, [M+H]⁺; ¹H NMR (400 MHz, DMSO) δ 10.97 (s, 1H), 10.07 (s, 1H), 8.28 (s, 2H), 6.98 (d, J=2.0 Hz, 1H), 6.90 (s, 1H), 6.53-6.52 (m, 1H), 5.01-4.97 (m, 1H), 3.66-3.61 (m, 8H), 3.28-3.21 (m, 1H), 2.87-2.67 (m, 2H), 2.40 (s, 3H), 2.33 (s, 3H).

Using the procedure described for Compound 370, above, an additional compound described herein was prepared by substituting the appropriate starting materials, suitable reagents, and reaction conditions, obtaining compounds such as those selected from:

| Cpd | Data |
|---|---|
| 276 | MS m/z = 475.2 [M + H]⁺; ¹H NMR (DMSO-d, 400 MHz) δ |
| | 10.58 (s, 1H), 9.34 (s, 1H), 8.78 (d, J = 4.8 Hz, 1H), 7.33 (s, 1H), |
| | 7.12 (d, J = 4.8 Hz, 1H), 7.02 (t, J = 7.5 Hz, 1H), 6.71 (s, 1H), |
| | 6.67 (t, J = 7.3 Hz, 1H), 6.35 (s, 1H), 4.83 (s, 1H), 3.33-3.19 (m, |
| | 1H), 2.79 (dd, J = 10.4, 5.0 Hz, 2H), 2.29 (s, 3H), 2.26 (s, 3H). |

### Example 26

### Preparation of Compound 272

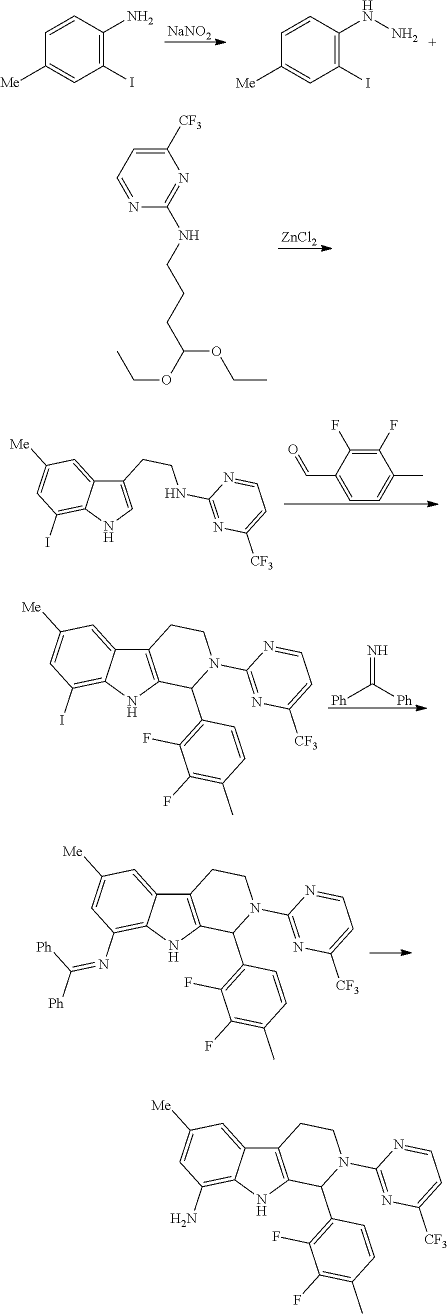

Step 1: 2-iodo-4-methyl-aniline (3.0 g, 12.9 mmol) was suspended in an ice-cold aqueous solution of concentrated hydrochloric acid (10 mL, 121.8 mmol). A solution of sodium nitrite (0.850 g, 12.3 mmol) in 5 mL water was added dropwise. After 45 min stirring at 0° C., a solution of tin dichloride (5.3 g, 27.7 mmol) in 6 mL of concentrated HCl was added to the mixture. The reaction mixture was allowed to warm to room temp over 3 hours, then stirred at RT for 12 h hours. After that, the suspension was diluted with water and the aqueous layer was washed with DCM twice. The aqueous layer was basified with 6 M NaOH solution until pH >10. The suspension was extracted 2× with DCM and 1× w/ EtOAc, dried over MgSO4 and filtered. The crude material was purified by silica gel chromatography, (2-iodo-4-methyl-phenyl)hydrazine was isolated as a yellow solid (2.1 g 68% yield).

Step 2: Placed (2-iodo-4-methyl-phenyl)hydrazine (500 mg, 2.0 mmol), N-(4,4-diethoxybutyl)-4-(trifluoromethyl)pyrimidin-2-amine (650 mg, 2.1 mmol) and zinc chloride (300 mg, 2.2 mmol) in a 50 mL RBF and to it was added 1.5 mL of dioxane. Placed the solution on a hot plate at 180° C. Allowed the solvent to evaporate until a black tar was left which was stirred at 180° C. for 5 min. The mixture was cooled to RT and purified by silica gel chromatography. Obtained N-[2-(7-iodo-5-methyl-1H-indol-3-yl)ethyl]-4-(trifluoromethyl)pyrimidin-2-amine as a yellow solid 420 mg, 47% y.

Step 3: To a mixture of N-[2-(7-iodo-5-methyl-1H-indol-3-yl)ethyl]-4-(trifluoromethyl)pyrimidin-2-amine (420 mg, 0.94 mmol,) and 2,3-difluoro-4-methyl-benzaldehyde (175 mg, 1.13 mmol) in a vial was added trifluoroacetic acid (0.9 mL, 1.13 mmol) and flushed with argon. The mixture was dissolved in 4 mL of DCE and stirred at 80° C. overnight.

The reaction mixture was quenched with 5 mL of saturated aqueous solution of NaHCO₃ and the aqueous layer was extracted with DCM. The organic fractions were dried over MgSO₄ and concentrated in vacuo. Purification by silica gel chromatography obtained 1-(2,3-difluoro-4-methyl-phenyl)-8-iodo-6-methyl-2-[4-(trifluoromethyl)pyrimidin-2-yl]-1,3,4,9-tetrahydropyrido[3,4-b]indole as a white solid, (320 mg, 58% yield).

Step 4: 1-(2,3-difluoro-4-methyl-phenyl)-8-iodo-6-methyl-2-[4-(trifluoromethyl)pyrimidin-2-yl]-1,3,4,9-tetrahydropyrido[3,4-b]indole (160 mg, 0.27 mmoL) was placed in a vial and sodium tert-butoxide (93 mg, 0.98 mmol), XPHOS PD G3 (4.8 mg, 0.054 mmol) and diphenylmethanimine (98 mg, 0.54 mmol) were added. The vial was flushed with argon, then 5 mL of toluene was added, and the mixture was stirred at 100° C. overnight.

The mixture was filtered through a silica gel plug with ethyl acetate, then concentrated and used in the next step (yellow oil, 175 mg, 76% yield).

Step 5: Dissolved N-[1-(2,3-difluoro-4-methyl-phenyl)-6-methyl-2-[4-(trifluoromethyl)pyrimidin-2-yl]-1,3,4,9-tetrahydropyrido[3,4-b]indol-8-yl]-1,1-diphenyl-methanimine (175 mg, 0.28 mmol) in methanol, added 2.0 mL of 4 N dioxane in HCl and 0.40 mL of H₂O, stirred for 4 hours at RT, then concentrated in vacuo and purified by prep HPLC. Obtained Compound 272 as a white solid (19 mg, 15% yield). MS m/z=475.2 [M+H]⁺; ¹H NMR (DMSO-d, 400 MHz) δ 10.28 (s, 1H), 8.78 (d, J=4.9 Hz, 1H), 7.20 (s, 1H), 7.12 (d, J=4.9 Hz, 1H), 7.04 (t, J=7.5 Hz, 1H), 6.84 (t, J=7.3 Hz, 1H), 6.53 (s, 1H), 6.19 (d, J=1.4 Hz, 1H), 4.90 (s, 1H), 4.83 (s, 2H), 3.42 (s, 1H), 2.86-2.74 (m, 2H), 2.26 (s, 3H), 2.25 (s, 3H).

Using the procedure described for Compound 272 above, additional compounds described herein were prepared by substituting the appropriate starting materials, suitable reagents, and reaction conditions, obtaining compounds such as those selected from:

| Cpd | Data |
|---|---|
| 231 | MS m/z = 456.0 [M − H]⁻; ¹H NMR (400 MHz, DMSO) δ 10.65 (s, 1H), 8.83-8.72 |
| | (m, 1H), 7.25-7.16 (m, 4H), 7.09 (d, J = 4.8 Hz, 1H), 7.06-6.94 (m, 1H), 6.73 (d, |
| | J = 1.6 Hz, 1H), 6.33 (d, J = 1.6 Hz, 1H), 5.24 (s, 2H), 4.95-4.76 (m, 1H), 3.26- |
| | 3.21 (m, 1H), 2.83-2.71 (m, 2H), 2.27 (s, 3H). |
| 248 | MS m/z = 494.0 [M + H]⁺; ¹H NMR (DMSO-d, 400 MHz) δ 10.60 (s, 1H), 8.78 (d, J = |
| | 4.8 Hz, 1H), 7.21 (s, 1H), 7.13 (d, J = 4.9 Hz, 1H), 7.05 (t, J = 7.4 Hz, 1H), 6.85 |
| | (t, J = 7.9 Hz, 1H), 6.74 (d, J = 1.8 Hz, 1H), 6.34 (d, J = 1.9 Hz, 1H), 5.25 (s, 2H), |
| | 4.90 (m, 1H), 3.43 (m, 1H), 2.86-2.74 (m, 2H), 2.26 (d, J = 1.2 Hz, 3H). |
| 304 | MS m/z = 455.0 [M + H]⁺; ¹H NMR (400 MHz, DMSO) δ 10.57 (s, 1H), 7.25 (s, 1H), |
| | 7.05 (t, J = 7.2 Hz, 1H), 6.85 (t, J = 10.0 Hz, 1H), 6.73 (d, J = 1.6 Hz, 1H), 6.34 (d, |
| | J = 1.6 Hz, 1H), 5.23-5.21 (m, 1H), 4.98-4.94 (m, 1H), 3.36-3.28 (m, 1H), 2.82- |
| | 2.67 (m, 2H), 2.34 (s, 6H), 2.26 (d, J = 1.2 Hz, 3H). |
| 321 | MS m/z = 491.0 [M + H]⁺; ¹H NMR (DMSO-d, 400 MHz) δ 10.56 (s, 1H), 8.16 (d, J = |
| | 2.4 Hz, 1H), 7.46 (dd, J = 9.2, 2.0 Hz, 1H), 6.99 (s, 1H), 6.81 (d, J = 9.2 Hz, 1H), |
| | 6.73 (d, J = 2.0 Hz, 1H), 6.33 (d, J = 2.0 Hz, 1H), 5.25 (s, 1H), 5.01-4.96 (m, 1H), |
| | 3.67-3.65 (m, 4H), 3.42-3.29 (m, 4H), 3.25-3.17 (s, 1H), 2.80-2.67 (m, 2H), |
| | 2.40 (s, 3H), 2.32 (s, 3H). |
| 351 | MS m/z = 476.0 [M + H]⁺; ¹H NMR (400 MHz, DMSO) δ 10.54 (s, 1H), 8.29 (d, J = |
| | 4.8 Hz, 1H), 8.15 (d, J = 2.4 Hz, 1H), 7.45 (dd, J = 8.8, 2.4 Hz, 1H), 6.98 (s, 1H), |
| | 6.81 (d, J = 2.0 Hz, 1H), 6.71 (d, J = 2.0 Hz, 1H), 6.56 (d, J = 5.2 Hz, 1H), 6.32 (d, |
| | J = 2.0 Hz, 1H), 5.25 (br, 2H), 4.97-4.93 (m, 1H), 3.67-3.65 (m, 4H), 3.41-3.38 |
| | (m, 4H), 3.22-3.15 (m, 1H), 2.76-2.67 (m, 2H), 2.34 (s, 3H). |

### Example 27

### Preparation of 4-fluoro-5-methyl-1H-indole

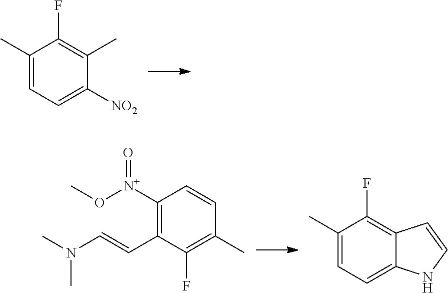

Step 1: To a RBF was added 2-fluoro-1,3-dimenthyl-4-nitro-benzene (5.0 g, 30 mmol), N,N-dimethylformamide dimethyl acetal (12.0 g, 101 mmol), triethylamine (10.0 g, 98.8 mmol) and N,N-dimethylformamide (15.0 mL). The mixture was stirred at 100° C. for 24 h, cooled by pouring into ice water, and extracted with ethyl acetate. The organic phase was washed with brine, dried over MgSO₄, then concentrated in vacuo to obtain (E)-2-(2-fluoro-3-methyl-6-nitro-phenyl)-N,N-dimethyl-ethenamine (9.6 g, 68% yield).

Step 2: To a RBF was added (E)-2-(2-fluoro-3-methyl-6-nitro-phenyl)-N,N-dimethyl-ethenamine (9.6 g, 30 mmol), iron powder (17.0 g, 304 mmol), acetic acid (50.0 mL, 873 mmol), and toluene (100.0 mL). The mixture was stirred at 100° C. for 4 h. The solid was collected by filtration and further purified by silica gel chromatography to afford 4-fluoro-5-methyl-1H-indole (1.8 g, 40% yield).

### Example 28

### Preparation of Compound 106

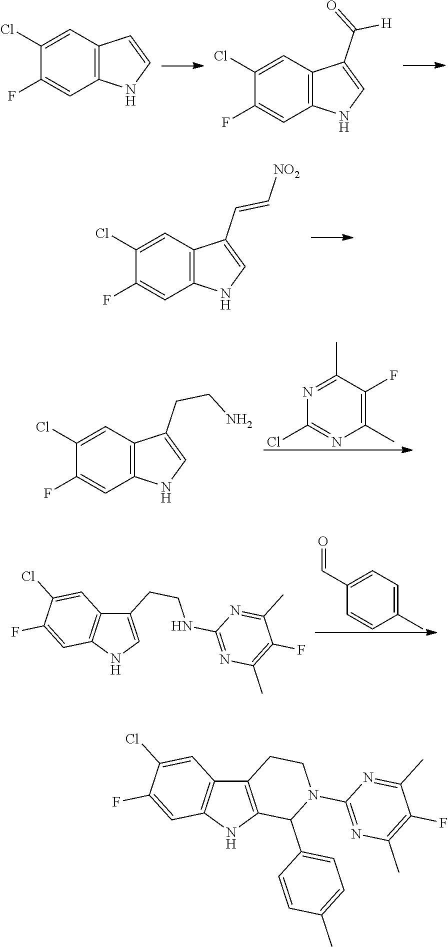

Step 1: Phosphoryl chloride (24.0 g, 156 mmol) was added to DMF (50 mL) solution at 0° C. under N₂ atmosphere. The mixture was warmed to RT and stirred for 1 Hour, then mixture then cooled back to 0° C., and 5-chloro-6-fluoro-1H-indole (20.4 g, 120 mmol) in 350 mL DMF was added to the mixture drop-wise. The reaction was warmed to RT and stirred for 2 Hours. The reaction was cooled to 0° C. quenched with 20 mL of 15% aq. NaOH solution, followed by 50 mL of water. The mixture was heated to 100° C. for 1 h and cooled to rt, resulting in a slurry. This was filtered and the solids were washed with water and then dried to give the product 5-chloro-6-fluoro-1H-indole-3-carbaldehyde (19.6 g, 99.2 mmol, 82.3% yield) as a yellow solid. MS m/z 198.0 [M+H]⁺.

Step 2: 5-chloro-6-fluoro-1H-indole-3-carbaldehyde (43.9 g, 222 mmol) and ammonium acetate (22.31 g, 289.4 mmol) were dissolved in 300 mL of nitromethane and stirred at 75° C. for 2 Hours. The mixture was cooled to 0° C. resulting in precipitate formation. The solid was collected by filtration, washed by water, and dried under vacuum to provide the product 5-chloro-6-fluoro-3-[(E)-2-nitrovinyl]-1H-indole (34.8 g, 145 mmol, 65.1% yield) as a yellow solid. MS m-z 241.0 [M+H]⁺.

Step 3: Lithium aluminum hydride (15.0 g, 383 mmol) was placed in a flask under N2 atmosphere and suspended in 250 mL of THF. The mixture was cooled to 0° C. and a solution of 5-chloro-6-fluoro-3-[(E)-2-nitrovinyl]-1H-indole (15.0 g, 62.3 mmol) in 50 mL of THF was added drop-wise. After addition the mixture was heated to 65° C. and stirred for 2 Hr. Upon completion the mixture was cooled down to 0° C. and quenched by addition of 15 mL water followed by addition of 15 mL of 15% aq. NaOH solution. Stirred the mixture at RT for 30 min, then MgSO₄ was added and the solids were removed by filtration. The solution was concentrated under reduced pressure providing 2-(5-chloro-6-fluoro-1H-indol-3-yl)ethanamine (B, 12.11 g, 56.95 mmol, 91.4% yield) as a yellow oil. MS m/z 213.4 [M+H]⁺.

Step 4: A mixture of 2-(5-chloro-6-fluoro-1H-indol-3-yl)ethanamine (300 mg, 1.4 mmol), 2-chloro-5-fluoro-4,6-dimethyl-pyrimidine (270 mg, 1.68 mmol) and Hunig's base (55 mg, 0.43 mmol) in 1-butanol (3 mL, 32.7 mmol) was stirred at 120° C. for 16 hours. Then the solvent was removed under reduced pressure and the mixture was purified by silica gel chromatography to provide N-[2-(5-chloro-6-fluoro-1H-indol-3-yl)ethyl]-5-fluoro-4,6-dimethyl-pyrimidin-2-amine (270 mg, 0.80 mmol, 56.8% yield) as a light yellow solid. MS m/z 213.4 [M+H]⁺.

Step 5: To a stirred suspension of N-[2-(5-chloro-6-fluoro-1H-indol-3-yl)ethyl]-5-fluoro-4,6-dimethyl-pyrimidin-2-amine (50 mg, 0.15 mmol) in 3 mL of s-BuOH was added 4-methylbenzaldehyde (B, 22 mg, 0.18 mmol) and TsOH (16 mg, 0.09 mmol). The suspended mixture was heated to reflux for 1 Hour, then cooled to RT and concentrated. The crude material was purified by prep-HPLC to give the Compound 106 (26 mg, 0.059 mmol, 39.90% yield) as a white solid. MS: m/z=439.0, [M+H]⁺; ¹H NMR (400 MHz, DMSO) δ 11.26 (s, 1H), 7.62 (d, J=7.6 Hz, 1H), 7.29 (d, J=10.4 Hz, 1H), 7.18-7.12 (m, 4H), 7.02 (s, 1H), 4.82-4.74 (m, 1H), 3.15-3.08 (m, 1H), 2.81-2.75 (m, 2H), 2.32 (d, J=2.4 Hz, 6H), 2.26 (s, 3H).

Using the procedure described for Compound 106 above, additional compounds described herein were prepared by substituting the appropriate starting materials, suitable reagents, and reaction conditions obtaining compounds such as those selected from:

| Cpd | Data |
|---|---|
| 53 | ESI-MS: m/z = 455.0. [M + H]⁺; ¹H NMR (DMSO, 400 MHz), δ 11.31 (s, 1H), 8.41 |
| | (d, J = 4.0 Hz, 1H), 7.66 (d, J = 7.6 Hz, 1H), 7.31 (d, J = 10.4 Hz, 1H), 7.23 (s, 4H), |
| | 7.02 (br, 1H), 7.81 (d, J = 5.2 Hz, 1H), 4.82 (br, 1H), 3.26-3.18 (m, 1H), 2.89-2.75 |
| | (m, 3H), 1.16 (d, J = 6.8 Hz, 6H). |
| 99 | MS m/z = 489.1, [M + H]⁺; ¹H NMR (400 MHz, DMSO) δ 11.35 (s, 1H), 8.90-8.73 |
| | (m, 2H), 7.67 (d, J = 7.6 Hz, 1H), 7.32 (d, J = 10.4 Hz, 1H), 7.26-7.21 (m, 4H), |
| | 7.17 (s, 1H), 4.95 (dd, J = 13.6, 5.2 Hz, 1H), 3.30-3.22 (m, 1H), 2.91-2.76 (m, |
| | 3H), 1.16 (d, J = 7.2 Hz, 6H). |
| 100 | MS m/z = 470.2, [M + H]⁻; ¹H NMR (400 MHz, DMSO) δ 11.34 (s, 1H), 8.80 (d, J = |
| | 4.8 Hz, 1H), 7.85 (d, J = 8.4 Hz, 2H), 7.70 (d, J = 7.2 Hz, 1H), 7.51 (d, J = 8.4 Hz, |
| | 2H), 7.38 (d, J = 10.4 Hz, 1H), 7.16-7.06 (m, 2H), 4.96-4.89(m, 1H), 3.31-3.22 |
| | (m, 1H), 2.93-2.80 (m, 2H). |
| 101 | MS m/z = 429.0, [M + H]⁺; ¹H NMR (400 MHz, DMSO) δ 1.28 (s, 1H), 8.64 (s, 2H), |
| | 8.54 (s, 2H), 7.70 (d, J = 4.0 Hz, 1H), 7.37 (d, J = 12.0 Hz, 1H), 6.93 (s, 1H), 4.91 |
| | (dd, J = 16.0, 4.0 Hz, 1H), 3.31-3.24 (m, 1H), 2.93-2.80 (m, 2H), 2.59 (s, 3H). |
| 102 | MS m/z = 458.2, [M + H]⁺; ¹H NMR (400 MHz, DMSO) δ 11.20 (s, 1H), 8.52 (s, |
| | 2H), 8.26 (s, 2H), 7.67 (d, J = 7.2 Hz, 1H), 7.32 (d, J = 10.4 Hz, 1H), 6.78 (s, 1H), |
| | 4.93-4.88 (m, 1H), 3.31-3.26 (m, 1H), 3.08 (s, 6H), 2.91-2.76 (m, 2H). |
| 103 | MS m/z = 423.0, [M + H]⁻; ¹H NMR (400 MHz, DMSO) δ 11.35 (s, 1H), 8.81 (d, J = |
| | 4.8 Hz, 1H), 7.93 (d, J = 8.3 Hz, 2H), 7.71 (d, J = 7.2 Hz, 1H), 7.60 (d, J = 8.2 Hz, |
| | 2H), 7.37 (d, J = 10.2 Hz, 1H), 7.27-7.08 (m, 2H), 5.01-4.85 (m, 1H), 3.32-3.24 (m, |
| | 1H), 3.19 (s, 3H), 2.94-2.80 (m, 2H). |
| 105 | MS m/z = 429.1, [M + H]⁺; ¹H NMR (400 MHz, DMSO) δ 11.30 (s, 1H), 8.37 (d, J = |
| | 1.6 Hz, 1H), 7.65 (d, J = 7.2 Hz, 1H), 7.33-7.30 (m, 3H), 7.19-7.15 (m, 2H), 7.04 |
| | (s, 1H), 4.81-4.78 (m, 1H), 3.15-3.12 (m, 1H), 2.80-2.77 (m, 2H), 2.37 (d, J = 2.4 |
| | Hz, 3H). |
| 107 | MS m/z = 475.3, [M + H]⁺; ¹H NMR (400 MHz, DMSO) δ 11.34 (s, 1H), 8.56 (s, |
| | 2H), 7.92 (d, J = 8.4 Hz, 2H), 7.68 (d, J = 7.4 Hz, 1H), 7.58 (d, J = 8.3 Hz, 2H), 7.36 |
| | (d, J = 10.1 Hz, 1H), 7.09 (s, 1H), 4.86-4.77 (m, 1H), 3.26-3.20 (m, 1H), 3.19 (s, 3H), |
| | 2.87-2.79 (m, 2H). |
| 108 | MS m/z = 481.0, [M + H]⁻; ¹H NMR (400 MHz, DMSO) δ 11.26 (s, 1H), 8.79 (d, |
| | J = 4.8 Hz, 1H), 7.69 (d, J = 7.6 Hz, 1H), 7.45-7.39 (m, 1H), 7.36-7.31 (m, 2H), |
| | 7.17-7.12 (m, 2H), 6.99-6.87 (m, 1H), 4.91-4.89 (m, 1H), 3.41-3.39 (m, 1H), |
| | 2.94-2.80 (m, 2H). |
| 112 | MS m/z = 460.9, [M + H]⁺; ¹H NMR (400 MHz, DMSO-d₆) δ 11.20 (s, 1H), 7.65 (d, |
| | J = 7.2 Hz, 1H), 7.42-7.35 (m, 1H), 7.31 (d, J = 10.0 Hz, 1H), 7.26 (s, 1H), 7.15- |
| | 7.09 (m, 1H), 6.85 (m, 1H), 4.84-4.75 (m, 1H), 3.30-3.21 (m, 1H), 2.84-2.77 (m, 2H), |
| | 2.30 (d, J = 2.4 Hz, 6H). |
| 115 | MS m/z = 524.2, [M + H]⁻; ¹H NMR (400 MHz, DMSO-d₆) δ 11.33 (s, 1H), 8.80 (d, |
| | J = 4.4 Hz, 2H), 7.80 (d, J = 8.4 Hz, 2H), 7.70 (d, J = 7.4 Hz, 1H), 7.51 (d, J = 8.4 |
| | Hz, 2H), 7.43-7.27 (m, 2H), 7.14 (d, J = 4.9 Hz, 2H), 4.93 (s, 1H), 3.31-3.22 (m, |
| | 1H), 2.96-2.77 (m, 2H). |
| 121 | MS m/z = 470.2, [M + H]⁻; ¹H NMR (400 MHz, DMSO) δ (s, 1H), 8.77 (d, J = 4.8 |
| | Hz, 1H), 7.75 (d, J = 8.4 Hz, 2H), 7.69 (d, J = 7.2 Hz, 1H), 7.54 (d, J = 8.4 Hz, 2H), |
| | 7.38 (d, J = 10.0 Hz, 1H), 7.28 (d, J = 4.4 Hz, 1H), 7.11 (br, 1H), 4.85 (br, 1H), |
| | 3.25-3.19 (m, 1H), 2.90-2.83 (m, 2H). |
| 122 | MS m/z = 491.1, [M + H]⁺; ¹H NMR (400 MHz, DMSO) δ 11.23 (s, 1H), 8.77 (d, |
| | J = 4.0 Hz, 1H), 8.07 (d, J = 2.0 Hz, 1H), 7.67 (d, J = 7.6 Hz, 1H), 7.39-7.36 (m, 1H), |
| | 7.30 (d, J = 10.0 Hz, 1H), 7.09 (d, J = 5.2 Hz, 1H), 6.98-6.85 (m, 1H), 6.59 (d, J = 8.8 |
| | Hz, 1H), 5.01-4.86 (m, 1H), 3.32-3.28 (m, 1H), 2.97 (s, 6H), 2.83-2.75 (m, 2H). |
| 123 | MS m/z = 425.1, [M + H]⁺; ¹H NMR (400 MHz, DMSO) δ 11.28 (s, 1H), 8.36 (d, |
| | J = 1.2 Hz, 1H), 7.63 (d, J = 7.6 Hz, 1H), 7.30 (d, J = 10.0 Hz, 1H), 7.18-7.13 (m, 4H), |
| | 7.01 (s, 1H), 4.80-4.76 (m, 1H), 3.17-3.13 (m, 1H), 2.79-2.77 (m, 2H), 2.36 (d, |
| | J = 2.4 Hz, 3H), 2.27 (s, 3H) |
| 124 | MS m/z = 447.2, [M + H]⁺; ¹H NMR (400 MHz, DMSO) δ 11.21 (s, 1H), 8.37 (d, |
| | J = 1.6 Hz, 1H), 7.66 (d, J = 7.2 Hz, 1H), 7.43-7.37 (m, 1H), 7.34-7.29 (m, 2H), 7.16- |
| | 7.11 (m, 1H), 6.84-6.81 (m, 1H), 4.80-4.76 (m, 1H), 3.31-3.24 (m, 1H), 2.83-2.78 |
| | (m, 2H), 2.35 (d, J = 2.4 Hz, 3H). |
| 125 | MS m/z = 461.1, [M + H]⁺; ¹H NMR (400 MHz, DMSO) δ 11.19 (s, 1H), 8.37 (d, |
| | J = 1.2 Hz, 1H), 7.65 (d, J = 7.6 Hz, 1H), 7.31 (d, J = 10.0 Hz, 1H), 7.25 (s, 1H), 7.03- |
| | 6.99 (m, 1H), 6.72-6.68 (m, 1H), 4.80-4.76 (m, 1H), 3.31-3.24 (m, 1H), 2.81-2.77 |
| | (m, 2H), 2.35 (d, J = 2.4 Hz, 3H), 2.25 (d, J = 1.6 Hz, 3H). |
| 126 | MS m/z = 437.2, [M + H]⁺; ¹H NMR (400 MHz, DMSO) δ 11.21 (s, 1H), 8.28 (d, |
| | J = 4.8 Hz, 1H), 8.04 (d, J = 2.4 Hz, 1H), 7.64 (d, J = 7.2 Hz, 1H), 7.38-7.35 (m, 1H), |
| | 7.29 (d, J = 10.0 Hz, 1H), 7.00 (s, 1H), 6.59-6.55 (m, 2H), 4.96-4.92 (m, 1H), 3.21- |
| | 3.13 (m, 1H), 2.97 (s, 6H), 2.85-2.74 (m, 2H), 2.33 (s, 3H). |
| 127 | MS m/z 475.1, [M + H]⁺; ¹H NMR (400 MHz, DMSO) δ 11.34 (s, 1H), 8.56 (s, 2H), |
| | 7.92 (d, J = 8.4 Hz, 2H), 7.68 (d, J = 7.4 Hz, 1H), 7.58 (d, J = 8.3 Hz, 2H), 7.36 (d, |
| | J = 10.1 Hz, 1H), 7.09 (s, 1H), 4.86-4.77 (m, 1H), 3.26-3.20 (m, 1H), 3.19 (s, 3H), |
| | 2.87-2.79 (m, 2H). |
| 129 | MS m/z = 443.2, [M + H]⁺; ¹H NMR (400 MHz, DMSO) δ 11.21 (s, 1H), 7.65 (d, |
| | J = 7.6 Hz, 1H), 7.42-7.35 (m, 2H), 7.31 (d, J = 10.0 Hz, 1H), 7.15-7.11 (m, 1H), |
| | 6.88-6.83 (m, 1H), 6.49 (s, 1H), 4.94-4.89 (m, 1H), 3.31-3.21 (m, 1H), 2.80-2.75 |
| | (m, 2H), 2.26 (s, 6H) |
| 130 | MS m/z = 463.0, [M + H]⁺; ¹H NMR (400 MHz, DMSO) δ 11.23 (s, 1H), 8.53 (s, |
| | 2H), 7.66 (d, J = 8.0 Hz, 1H), 7.34 (d, J = 12.0 Hz, 1H), 7.27 (s, 1H), 7.03 (t, J = |
| | 8.0 Hz, 1H), 6.70 (t, J = 8.0 Hz, 1H), 4.81-4.77 (m, 1H), 3.29-3.24 (m, 1H), 2.82- |
| | 2.79 (m, 2H), 2.26 (s, 3H). |
| 131 | MS m/z = 449.0, [M + H]⁺; ¹H NMR (400 MHz, DMSO) δ 11.25 (s, 1H), 8.53 (s, |
| | 2H), 7.66 (d, J = 8.0 Hz, 1H), 7.42-7.39 (m, 1H), 7.36-7.33 (m, 2H), 7.18-7.13 (m, |
| | 1H), 6.84-6.81 (m, 1H),4.81 (dd, J = 12.0, 4.0 Hz, 1H), 3.30-3.25 (m, 1H), 2.85- |
| | 2.80 (m, 2H). |
| 132 | MS m/z = 457.2, [M + H]⁺; ¹H NMR (400 MHz, DMSO) δ 11.19 (s, 1H), 7.64 (d, |
| | J = 7.6 Hz, 1H), 7.36 (s, 1H), 7.30 (d, J = 10.0 Hz, 1H), 7.02-6.98 (m, 1H), 6.76-6.72 |
| | (m, 1H), 6.49 (s, 1H), 4.94-4.89 (m, 1H), 3.31-3.21 (m, 1H), 2.82-2.75 (m, 2H), 2.26 |
| | (s, 6H), 2.25 (s, 3H). |
| 133 | MS m/z = 481.0, [M + H]⁻; ¹H NMR (400 MHz, DMSO) δ 11.28 (s, 1H), 8.84 (s, |
| | 2H), 7.69 (d, J = 4.0 Hz, 1H), 7.48-7.41 (m, 2H), 7.37 (d, J = 8.0 Hz, 1H), 7.20- |
| | 7.15 (m, 1H), 6.87 (t, J = 8.0 Hz, 1H), 4.97 (dd, J = 20.0, 4.0 Hz, 1H), 3.38-3.32(m, |
| | 1H), 2.92-2.79(m, 2H). |
| 134 | MS m/z = 407.1, [M + H]⁺; ¹H NMR (400 MHz, DMSO) δ 11.29 (s, 1H), 8.29 (s, |
| | 2H), 7.62(d, J = 7.6 Hz, 1H), 7.30 (d, J = 10.0 Hz, 1H), 7.18-7.12 (m, 4H), 7.06 (s, |
| | 1H), 4.83-4.78 (m, 1H), 3.16-3.09 (m, 1H), 2.77-2.75 (m, 2H), 2.26 (s, 3H), 2.09 |
| | (s, 3H). |
| 136 | MS m/z = 443.2, [M + H]⁺; ¹H NMR (400 MHz, DMSO) δ 11.18 (s, 1H), 8.30 |
| | (s, 2H), 7.63 (d, J = 7.2 Hz, 1H), 7.33-7.31 (m, 2H), 7.03-6.99 (m, 1H), 6.69-6.65 |
| | (m, 1H), 4.81-4.77 (m, 1H), 3.26-3.19 (m, 1H), 2.78-2.76 (m, 2H), 2.25 (s, 3H), |
| | 2.09 (s, 3H). |
| 138 | MS m/z = 445.3, [M + H]⁻; ¹H NMR (400 MHz, DMSO) δ 11.20(s, 1H), 8.54 (s, |
| | 2H), 7.65 (d, J = 7.2 Hz, 1H), 7.33 (d, J = 10.0 Hz, 1H), 7.26 (s, 1H), 7.04-7.00 (m, |
| | 1H), 6.70-6.67 (m, 1H), 4.78-4.75 (m, 1H), 3.30-3.26 (m, 1H), 2.81-2.79 (m, 2H), |
| | 2.26 (s, 3H). |
| 139 | MS m/z = 443.2, [M + H]⁺; ¹H NMR (400 MHz, DMSO) δ 11.19 (s, 1H), 8.29 (d, J = |
| | 4.8 Hz, 1H), 7.64 (d, J = 7.2 Hz, 1H), 7.36 (s, 1H), 7.31(d, J = 10.4 Hz, 1H), 7.03- |
| | 6.99 (m, 1H), 6.74-6.70 (m, 1H), 6.60 (d, J = 5.2 Hz, 1H), 4.91-4.87 (m, 1H), 3.26- |
| | 3.23 (m. 1H), 2.80-2.76 (m, 2H), 2.32 (s, 3H), 2.25 (s, 3H). |
| 140 | MS m/z = 495.1, [M + H]⁻; ¹H NMR (400 MHz, DMSO) δ 11.24 (s, 1H), 8.78 (d, J = |
| | 4.8 Hz, 1H), 7.68 (d, J = 7.2 Hz, 1H), 7.34-7.23 (m, 2H), 7.14 (d, J = 5.2 Hz, 1H), |
| | 7.05-7.01 (m, 1H), 6.78-6.74 (m, 1H), 4.96-4.81 (m, 1H), 3.32-3.28 (m, 1H), |
| | 2.92-2.78 (m, 2H), 2.26 (s, 3H). |
| 141 | MS m/z = 452.0, [M + H]⁻; ¹H NMR (400 MHz, DMSO-d₆) δ 11.21 (s, 1H), 8.75 (d, |
| | J = 4.4 Hz, 1H), 7.67(d, J = 7.6 Hz, 1H), 7.34 (d, J = 10.0 Hz, 1H), 7.27-7.24 (m, |
| | 2H), 7.05-7.02 (m, 1H), 6.75-6.72 (m, 1H), 4.87-4.78 (m, 1H), 3.30-3.27 (m, |
| | 1H), 2.86-2.81 (m, 2H), 2.26 (s, 3H). |
| 142 | MS m/z = 495.1, [M + H]⁻; ¹H NMR (400 MHz, DMSO-d₆) δ 11.25 (s, 1H), 8.83 (s, |
| | 2H), 7.67 (d, J = 7.2 Hz, 1H), 7.39 (s, 1H), 7.35 (d, J = 10.4 Hz, 1H), 7.06-7.03 (m, |
| | 1H), 6.76-6.72 (m, 1H), 4.98-4.83 (m, 1H), 3.38-3.31 (m, 1H), 2.91-2.77 (m, 2H), |
| | 2.26 (s, 3H). |
| 143 | MS m/z = 473.4, [M + H]⁻; ¹H NMR (400 MHz, DMSO-d₆) δ 11.17 (s, 1H), 7.64 (d, |
| | J = 7.2 Hz, 1H), 7.29 (d, J = 10.0 Hz, 1H), 7.22 (s, 1H), 6.99 (t, J = 7.4 Hz, 1H), 6.71 |
| | (t, J = 6.8 Hz, 1H), 4.82-4.75 (m, 1H), 3.29-3.21 (m, 1H), 2.82-2.74 (m, 2H), 2.30 (d, |
| | J = 2.4 Hz, 6H), 2.25 (d, J = 1.2 Hz, 3H). |
| 145 | MS m/z = 463.9, [M + H]⁻; ¹H NMR (400 MHz, DMSO-d₆) 11.26 (s, 1H), 8.81 (d, |
| | J = 4.4 Hz. 1H), 8.56 (d, J = 2.8 Hz, 1H), 8.53-8.49 (m, 1H), 7.71 (d, J = 7.6 Hz, 1H), |
| | 7.61-7.57 (m, 1H), 7.37 (d, J = 10.0 Hz, 1H), 7.15 (d, J = 4.8 Hz, 1H), 7.08-6.96 (m, |
| | 1H), 5.01-4.90 (m, 1H), 3.44-3.37 (m, 1H), 2.97-2.77 (m, 2H). |
| 148 | MS m/z = 511.0, [M + H]⁺; ¹H NMR (400 MHz, DMSO-d₆) δ 11.23 (s, 1H), 7.68 (d, |
| | J = 7.2 Hz, 1H), 7.33-7.16 (m, 2H), 7.06 (s, 1H), 7.02 (t, J = 7.4 Hz, 1H), 6.78 (t, J = |
| | 7.2 Hz, 1H), 4.93 (br, 1H), 3.40-3.39 (m, 1H), 2.92-2.76 (m,2H), 2.45 (s, 3H), 2.25 |
| | (s, 3H). |
| 151 | MS m/z = 430.0, [M + H]⁻; ¹H NMR (400 MHz, DMSO-d₆) δ 11.28 (s, 1H), 7.65 (d, |
| | J = 7.6 Hz, 1H), 7.32 (d, J = 10.0 Hz, 1H), 7.33-6.92 (m, 6H), 4.95-4.69 (m, 1H), |
| | 3.31-3.14 (m, 1H), 2.88-2.75 (m, 2H), 2.46-2.35 (m, 3H), 2.27 (s, 3H). |
| 152 | MS m/z = 451.9, [M + H]⁻; ¹H NMR (400 MHz, DMSO-d₆) δ 11.23 (s, 1H), 7.68 (d, |
| | J = 7.2 Hz, 1H), 7.45-7.38 (m, 1H), 7.34 (d, J = 10.4 Hz, 1H), 7.31-7.23 (m, 1H), 7.20 |
| | (s, 1H), 7.17-7.12 (m, 1H), 6.88-6.85 (m, 1H), 4.93-4.74 (m, 1H), 3.31-3.24 (m, 1H), |
| | 2.90-2.76 (m, 2H), 2.41 (s, 3H). |
| 153 | MS m/z = 466.2, [M + H]⁻; ¹H NMR (400 MHz, DMSO-d₆) δ 11.20 (s, 1H), 7.67 (d, |
| | J = 7.2 Hz, 1H), 7.33 (d, J = 10.4 Hz, 1H), 7.29-7.15 (m, 2H), 7.02 (t, J = 7.4 Hz, 1H), |
| | 6.78-6.71 (m, 1H), 4.94-4.74 (m, 1H), 3.30-3.25 (m, 1H), 2.91-2.73 (m, 2H), 2.40 (s, |
| | 3H), 2.25 (s, 3H). |
| 154 | MS m/z = 526.8, [M + H]⁻; ¹H NMR (400 MHz, DMSO-d₆) δ 11.29 (s, 1H), 7.69 (d, |
| | J = 7.6 Hz, 1H), 7.53 (s, 1H), 7.32 (d, J = 10.0 Hz, 1H), 7.23-7.16 (m, 4H), 6.99 (s, |
| | 1H), 4.87 (dd, J = 13.6, 5.2 Hz, 1H), 3.43-3.36 (m, 1H), 2.97-2.80 (m, 2H), 2.27 (s, |
| | 3H). |
| 155 | MS m/z = 562.8, [M + H]⁻; ¹H NMR (400 MHz, DMSO-d₆) δ δ 11.26 (s, 1H), 7.71 (d, |
| | J = 7.6 Hz, 1H), 7.57 (s, 1H), 7.33(d, J = 10 Hz, 1H), 7.17 (s, 1H), 7.04 (t, J = 7.6 |
| | Hz, 1H), 6.82 (t, J = 7.0 Hz, 1H), 4.92-4.86 (m, 1H), 3.50 (dd, J = 11.6, 9.2 Hz, |
| | 1H), 3.00-2.95 (m, 1H), 2.90-2.85 (m, 1H), 2.26 (s, 3H). |
| 157 | MS m/z = 449.1, [M + H]⁻; ¹H NMR (400 MHz, DMSO-d₆) δ 11.28 (s, 1H), 7.73 (d, |
| | J = 7.2 Hz, 1H), 7.59 (s, 1H), 7.46-7.40 (m, 1H), 7.34 (d, J = 10.0 Hz, 1H), 7.21 (s, |
| | 1H), 7.18-7.13 (m, 1H), 6.94 (t, J = 6.8 Hz, 1H), 4.89 (dd, J = 13.6, 4.4 Hz, 1H), |
| | 3.55-3.49 (m, 1H), 3.02-2.83 (m, 2H). |
| 162 | MS m/z = 470.0, [M + H]⁻; ¹H NMR (400 MHz, DMSO-d₆) δ 11.21 (s, 1H), 8.93 (s, |
| | 1H), 7.68 (d, J = 8.0 Hz, 1H), 7.34 (d, J = 12.0 Hz, 1H), 7.14 (s, 1H), 7.04 (t, J = |
| | 8.0 Hz, 1H), 6.73 (t, J = 4.0 Hz, 1H), 4.75-4.71 (m, 1H), 3.41-3.36 (m, 1H), 2.89- |
| | 2.81 (m, 2H), 2.26 (s, 3H). |
| 163 | MS m/z = 435.0, [M + H]⁺; ¹H NMR (400 MHz, DMSO-d₆) δ 11.25 (s, 1H), 7.61 (d, |
| | J = 7.6 Hz, 1H), 7.28 (d, J = 10.4 Hz, 1H), 7.17-7.10 (m, 5H), 4.89-4.82 (m, 1H), |
| | 3.12-3.01 (m, 1H), 2.77-2.71 (m, 2H), 2.30 (s, 6H), 2.25 (s, 3H), 2.03 (s, 3H). |
| 164 | MS m/z = 531.0, [M + H]⁻; ¹H NMR (400 MHz, DMSO) δ 11.32 (s, 1H), 8.77 (s, |
| | 1H), 7.67 (d, J = 7.6 Hz, 1H), 7.31 (d, J = 10.4 Hz, 1H), 7.22 (t, J = 5 Hz, 4H), |
| | 7.09 (d, J = 5.2 Hz, 1H), 7.03 (br, 1H), 4.90 (br, 1H), 3.56 (s, 3H), 3.25 (t, J = |
| | 12 Hz, 1H), 2.79-2.91 (m, 4H), 2.59 (t, J = 7.6 Hz, 2H). |
| 165 | MS m/z = 483.1, [M + H]⁺; ¹H NMR (400 MHz, DMSO) δ 11.31 (s, 1H), 8.53 (s, |
| | 2H), 7.64 (d, J = 7.4 Hz, 1H), 7.30 (d, J = 10.1 Hz, 1H), 7.20 (s, 3H), 7.01 (s, 2H), |
| | 4.77 (d, J = 13.4 Hz, 1H), 3.57 (s, 3H), 3.21-3.14 (m, 1H), 2.89-2.72 (m, 4H), 2.60 |
| | (t, J = 7.5 Hz, 2H). |
| 169 | MS m/z = 471.0, [M + H]⁺; ¹H NMR (400 MHz, DMSO-d₆) δ 11.16 (s, 1H), 7.63 (d, |
| | J = 7.6 Hz, 1H), 7.33-7.27 (m, 2H), 6.98 (t, J = 7.4 Hz, 1H), 6.72 (t, J = 7.7 Hz, |
| | 1H), 4.89-4.81 (m, 1H), 3.28-3.18 (m, 1H), 2.79-2.71 (m, 2H), 2.28 (s, 6H), 2.24 (s, |
| | 3H), 2.02 (s, 3H). |
| 170 | MS m/z = 488.1, [M + H]⁻; ¹H NMR (400 MHz, DMSO-d₆) δ 11.26 (s, 1H), 8.76- |
| | 8.75 (m, 1H), 7.65 (d, J = 7.2 Hz, 1H), 7.28 (d, J = 10.4 Hz, 1H), 7.12-7.10 (m, 2H), |
| | 7.07 (d, J = 4.8 Hz, 1H), 7.02-6.89 (m, 1H), 6.68-6.66 (m, 2H), 5.00-4.70 (m, 1H), |
| | 3.27-3.19 (m, 1H), 2.90-2.77 (m, 8H). |
| 171 | MS m/z = 455.0 [M − H]⁻; ¹H NMR (400 MHz, DMSO-d₆) δ 11.20 (s, 1H), 7.64 (d, |
| | J = 7.4 Hz, 1H), 7.40-7.21 (m, 3H), 7.16-7.04 (m, 1H), 6.91-6.73 (m, 1H), 4.86 |
| | (dt, J = 13.7, 3.5 Hz, 1H), 3.32-3.15 (m, 1H), 2.91-2.66 (m, 2H), 2.29 (s, 6H), |
| | 2.03 (s, 3H). |
| 172 | MS m/z = 559.0, [M + H]⁻; ¹H NMR (400 MHz, DMSO-d₆) δ 1.24 (s, 1H), 8.80 (d, |
| | J = 4.0 Hz, 1H), 7.70 (d, J = 8.0 Hz, 1H), 7.48 (t, J = 4.0 Hz, 1H), 7.36 (d, J = 8.0 |
| | Hz, 1H), 7.23 (s, 1H), 7.16 (d, J = 8.0 Hz, 1H), 6.88 (t, J = 8.0 Hz, 1H), 4.93-4.90 |
| | (m, 1H), 3.43-3.41 (m, 1H), 3.02-2.68 (m, 2H). |
| 174 | MS m/z = 441.0 [M + H]⁺; ¹H NMR (400 MHz, DMSO) δ(ppm), 11.13(s, 1H), |
| | 8.77(s, 1H), 7.21(d, J = 8.0 Hz, 2H), 7.16(d, J = 8.0 Hz, 2H), 7.09 (d, J = 4.8 Hz, 1H), |
| | 7.03-7.00 (m, 2H), 6.90 (t, J = 7.8 Hz, 1H), 4.99-4.71(m, 1H), 3.31-3.21(m,1H), 3.03- |
| | 2.93(m, 2H), 2.27(s, 6H). |
| 175 | MS m/z = 434.0, [M + H]⁻; ¹H NMR (400 MHz, DMSO) δ 11.30 (s, 1H), 8.92 (s, |
| | 1H), 7.66 (d, J = 8.0 Hz, 1H), 7.33 (d, J = 12.0 Hz, 1H), 7.19-7.15(m, 4H), 6.91 (s, |
| | 1H), 4.74-4.71(m, 1H), 3.34-3.21 (m, 1H), 2.87-2.80 (m, 2H), 2.28 (s, 3H). |
| 183 | MS m/z = 456.0, [M + H]⁻; ¹H NMR (400 MHz, DMSO) δ 11.23 (s, 1H), 8.94 (s, |
| | 1H), 7.69 (d, J = 8.0 Hz, 1H), 7.44 (q, J = 8.0 Hz, 1H), 7.36 (t, J = 12.0 Hz, 1H), |
| | 7.19-7.13 (m, 2H), 6.85 (m, J = 8.0 Hz, 1H), 4.74 (dd, J = 16.0, 4.0 Hz, 1H), 3.39- |
| | 3.34(m, 1H), 2.91-2.82 (m, 2H). |
| 184 | MS m/z = 484.1, [M + H]⁺; ¹H NMR (400 MHz, DMSO) δ 11.22 (s, 1H), 8.80 (d, J = |
| | 4.0 Hz, 1H), 7.83 (dd, J = 16.0, 8.0 Hz, 1H), 7.71 (d, J = 8.0 Hz, 1H), 7.36 (d, J = |
| | 12.0 Hz, 1H), 7.16 (d, J = 4.0 Hz, 2H), 7.11 (d, J = 12.0, 4.0 Hz, 1H), 4.93-4.90 (m, |
| | 1H), 3.42-3.39 (m, 1H), 2.95-2.78(m, 2H). |
| 188 | MS m/z = 538.1, [M + H]⁻; ¹H NMR (400 MHz, DMSO) δ 11.31 (s, 1H), 7.87 (d, J = |
| | 8.8 Hz, 2H), 7.72 (d, J = 7.2 Hz, 1H), 7.60 (s, 1H), 7.52 (d, J = 8.4 Hz, 2H), 7.38 (d, |
| | J = 7.8 Hz, 1H), 7.05 (s, 1H), 4.88 (dd, J = 13.2, 4.4 Hz, 1H), 3.40-3.37 (m, 1H), |
| | 2.99-2.84 (m, 2H). |
| 192 | MS m/z = 477.0 [M + H]⁺; ¹H NMR (400 MHz, DMSO-d₆) δ 11.08 (s, 1H), 8.78 (d, |
| | J = 4.8 Hz, 1H), 7.26 (s, 1H), 7.14 (d, J = 4.8 Hz, 1H), 7.05-7.00 (m, 2H), 6.93-6.89 |
| | (m, 1H), 6.80-6.76 (m, 1H), 4.93-4.83 (m, 1H), 3.45-3.39 (m, 1H), 3.01-2.97 (m, |
| | 2H), 2.26 (s, 6H) |
| 201 | MS m/z = 441.9 [M + H]⁺; ¹H NMR (400 MHz, DMSO) δ 11.39 (s, 1H), 8.55 (s, |
| | 2H), 8.29 (s, 2H), 7.15(t, J = 8 Hz, 2H), 6.77 (s, 1H), 4.88 (dd, J = 4.2 Hz, 1H), |
| | 3.33 (s, 1H), 3.08 (s, 6H), 2.94-3.03 (m, 2H). |
| 211 | MS m/z = 482.0 [M −H]⁻; ¹H NMR (400 MHz, DMSO) δ 11.37 (s, 1H), 8.55 (s, 2H), |
| | 8.34 (s, 2H), 7.13-7.17 (m, 2H), 6.78 (s, 1H), 9.78 (dd, J = 4.2 Hz, 1H), 3.61-3.67 |
| | (m, 8H), 3.35 (s, 1H), 2.95-3.00 (m, 2H) |
| 212 | MS m/z = 412.0 [M − H]⁻; ¹H NMR (400 MHz, DMSO) δ 11.40 (s, 1H), 8.54 (s, 2H), |
| | 8.16 (s, 2H), 7.13-7.18 (m, 2H), 6.78 (s, 1H), 6.72 (s, 2H), 4.86 (dd, J = 4 Hz, 1H), |
| | 3.33 (s, 1H), 2.94-2.98 (m, 2H). |
| 214 | MS m/z = 470.0 [M − H]⁻; ¹H NMR (400 MHz, DMSO) δ 11.42 (s, 1H), 8.94 (s, 1H), |
| | 7.18-7.12 (m, 3H), 7.05 (t, J = 8.0 Hz, 1H), 6.77 (t, J = 8.0 Hz, 1H), 4.73 (d, J = |
| | 12.0 Hz, 1H), 3.44-3.34 (m, 1H), 3.04-2.97 (m, 2H), 2.27 (s, 3H). |
| 215 | MS m/z = 484.1, [M − H]; ¹H NMR (400 MHz, DMSO) δ 11.30 (s, 1H), 7.86 (s, 1H), |
| | 7.68 (d, J = 8.0 Hz, 1H), 7.35 (d, J = 12.0 Hz, 1H), 7.18 (s, 4H), 6.92 (s, 1H), 4.75 |
| | (dd, J = 12.0, 4.0 Hz, 1H), 3.31-3.25 (m, 1H), 2.92-2.79 (m, 2H), 2.28 (s, 3H). |
| 216 | MS m/z = 477.0, [M − H]; ¹H NMR (400 MHz, DMSO) δ 11.22 (s, 1H), 7.90 (s, 1H), |
| | 7.70 (d, J = 4.0 Hz, 1H), 7.36 (d, J = 8.0 Hz, 1H), 7.12 (s, 1H), 7.05 (t, J = 8.0 Hz, |
| | 1H), 6.77 (t, J = 8.0 Hz, 1H), 4.78 (dd, J = 12.0, 4.0 Hz, 1H), 3.43-3.38 (m, 1H), |
| | 3.95-2.84 (m, 2H), 2.27 (s, 3H). |
| 217 | MS m/z = 434.0 [M − H]⁻; ¹H NMR (400 MHz, DMSO) δ 11.50 (s, 1H), 8.92 (s, 1H), |
| | 7.20-7.11 (m, 6H), 6.93 (s, 1H), 4.72 (d, J = 16.0 Hz, 1H), 3.34-3.24 (m, 1H), |
| | 3.02-2.96 (m, 2H), 2.28 (s, 3H). |
| 225 | MS m/z = 462.0 [M − H]⁻; ¹H NMR (400 MHz, DMSO) δ 11.41 (s, 1H), 8.79 (d, J = |
| | 4.4 Hz, 1H), 8.20 (s, 2H), 7.19-7.12 (m, 3H), 6.79 (s, 3H), 4.99 (br, 1H), 3.44 - |
| | 3.37 (m, 1H), 3.09-2.93 (m, 2H). |
| 226 | MS m/z = 466.0 [M + H]⁺; ¹H NMR (400 MHz, DMSO) δ 11.47 (s, 1H), 8.81 (d, J = |
| | 4.0 Hz, 1H), 8.22 (d, J = 4.0 Hz, 1H), 7.64 (t, J = 8.0 Hz, 1H), 7.34-7.31 (m, 1H), |
| | 7.25 (s, 1H), 7.20-7.13 (m, 3H), 4.89 (d, J = 12.0 Hz, 1H), 3.32-3.29 (m, 1H), |
| | 3.03-2.99 (m, 2H). |
| 227 | MS m/z = 463.0, [M − H]; ¹H NMR (400 MHz, DMSO) δ 11.25 (s, 1H), 7.89 (s, 1H), |
| | 7.71 (d, J = 8.0 Hz, 1H), 7.45 (q, J = 8.0 Hz, 1H), 7.37 (d, J = 8.0 Hz, 1H), 7.25- |
| | 7.14(m, 2H), 6.89 (t, J = 8.0 Hz, 1H), 4.78 (dd, J = 12.0, 4.0 Hz, 1H), 3.39-7.35 |
| | (m, 1H), 2.97-2.81(m, 2H). |
| 246 | MS m/z = 481.0, [M − H]⁻; ¹H NMR (400 MHz, DMSO-d₆) δ 11.51 (s, 1H), 8.37 (d, |
| | J = 2.0 Hz, 1H), 7.22-7.10(m, 6H), 7.05 (s, 1H), 4.80-4.76(m, 1H), 4.37 (t, J = |
| | 4.0 Hz, 1H), 3.40-3.36(m, 2H), 3.22-3.15 (m, 1H), 3.01-2.91(m, 2H), 2.56-2.54 (m, |
| | 2H), 2.37 (d, J = 4.0 Hz, 3H), 1.57-1.53(m, 2H), 1.42-1.38(m, 2H). |

### Example 29

### Preparation of Compound 208

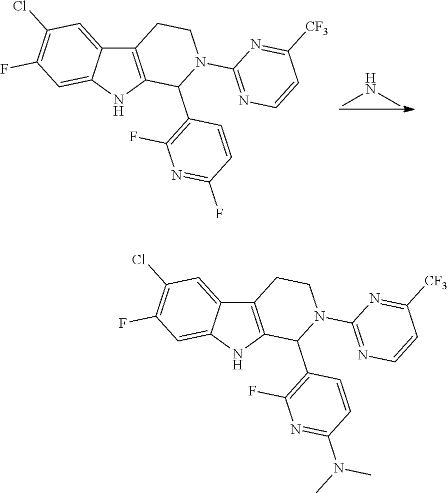

To a solution of 6-chloro-1-(2,6-difluoro-3-pyridyl)-7-fluoro-2-[4-(trifluoromethyl)pyrimidin-2-yl]-1,3,4,9-tetrahydropyrido[3,4-b]indole (70 mg, 0.1447 mmol) in THF (1 mL) was added a solution of dimethylamine (2 mol/L) in THF (1 mL, 2 mmol) followed by triethylamine (0.1 mL, 0.8 mmol). The mixture was stirred at 80° C. for 4 hr and then cooled to RT. The reaction mixture was concentrated under reduced pressure. The crude material was purified by prep-HPLC to give Compound 208 (D, 43 mg, 0.69 mmol 58% yield) as a white solid. MS m/z=507.0 [M−H]⁻; ¹H NMR (400 MHz, DMSO) δ 11.22 (s, 1H), 8.76 (d, J=4.8 Hz, 1H), 7.66 (d, J=7.6 Hz, 1H), 7.31 (d, J=10.0 Hz, 1H), 7.25 (t, J=9.4 Hz, 1H), 7.14-7.04, 2H), 6.42-6.39 (m, 1H), 4.96-4.82 (m, 1H), 3.31-3.28 (m, 1H), 2.97 (s, 6H), 2.90-2.73 (m, 2H).

Using the procedure described for Compound 208 above, additional compounds described herein were prepared by substituting the appropriate starting materials, suitable reagents, and reaction conditions, obtaining compounds such as those selected from:

| Cpd | Data |
|---|---|
| 158 | MS m/z = 507.1, [M + H]⁻; ¹H NMR (400 MHz, DMSO) δ 11.25 (br, 1H), 8.78 (d, J = |
| | 4.4 Hz, 1H), 7.93 (s, 1H), 7.68 (d, J = 7.2 Hz, 1H), 7.34-7.28 (m, 2H), 7.11 (d, J = 4.8 |
| | Hz, 1H), 6.99-6.82 (m, 1H), 5.06-4.86 (m, 1H), 3.52-3.45 (m, 1H), 2.98 (d, J = 2.0 Hz, |
| | 6H), 2.94-2.90 (m, 1H), 2.82-2.74 (m, 1H). |
| 173 | ESI-MS m/z = 524.0 [M − H]⁻; ¹H NMR (400 MHz, DMSO) δ 11.25 (s, 1H), 8.78 (d, J = |
| | 4.0 Hz, 1H), 7.67 (d, J = 8.0 Hz, 1H), 7.32 (d, J = 8.0 Hz, 1H), 7.24 (s, 1H), 7.12 (d, J = |
| | 4.0 Hz, 1H), 6.66 (d, J = 8.0 Hz, 2H), 4.92-4.81 (s, 1H), 3.42-3.32 (m, 1H), 2.90- |
| | 2.83 (m, 2H), 2.80 (s, 6H). |
| 210 | MS m/z = 509.0 [M + H]⁺, ¹H NMR (400 MHz, DMSO) δ 11.48 (s, 1H), 8.81 (d, J = |
| | 4.8 Hz, 1H), 7.40 (s, 1H), 7.33 (t, J = 8.4 Hz, 1H), 7.13-7.18 (m, 3H), 7.53 (dd, J = 3.4 |
| | Hz, 1H), 4.76 (d, J = 13.6 Hz, 1H), 3.32 (s, 1H), 2.97-2.99 (m, 2H), 2.92 (s, 6H). |

### Example 30

### Preparation of Compound 156

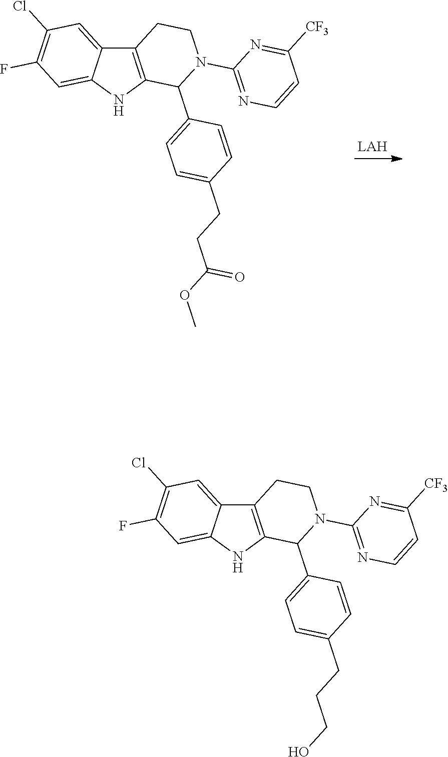 [SMILES: COC(=O)CCc1ccc(C2c3[nH]c4cc(F)c(Cl)cc4c3CCN2c2nccc(C(F)(F)F)n2)cc1 ; OCCCc1ccc(C2c3[nH]c4cc(F)c(Cl)cc4c3CCN2c2nccc(C(F)(F)F)n2)cc1]

To a suspension of lithium aluminum hydride (40 mg, 1.05 mmol) in THF (10 mL) was added a solution of methyl 3-[4-[6-chloro-7-fluoro-2-[4-(trifluoromethyl)pyrimidin-2-yl]-1,3,4,9-tetrahydropyrido[3,4-b]indol-1-yl]phenyl]propanoate (300 mg, 0.56 mmol) in THF (10 mL). The mixture was stirred at 25° C. for 2 Hr. The mixture was quenched by sequential addition of water (40 uL) and 10% aq. NaOH (40 uL). The suspension was stirred for 20 min and then dried with MgSO₄, filtered, and concentrated under reduced pressure. The crude product was purified by silica gel chromatography with eluent PE:EA=3:1 to provide Compound 156 (170 mg, 0.34 mmol, 59.81% yield) as a yellow oil. MS m/z=503.1, [M+H]⁻; ¹H NMR (400 MHz, DMSO) δ 11.32 (s, 1H), 8.77 (s, 1H), 7.67 (d, J=7.3 Hz, 1H), 7.31 (d, J=10.1 Hz, 1H), 7.20 (q, J=8.2 Hz, 4H), 7.13-6.94 (m, 2H), 4.90 (s, 1H), 4.46 (t, J=5.1 Hz, 1H), 3.42-3.35 (m, 2H), 3.29-3.19 (m, 1H), 2.92-2.75 (m, 2H), 2.62-2.53 (m, 2H), 1.71-1.61 (m, 2H).

Using the procedure described for Compound 156 above, additional compounds described herein were prepared by substituting the appropriate starting materials, suitable reagents, and reaction conditions, obtaining compounds such as those selected from:

| Cpd | Data |
|---|---|
| 159 | MS m/z = 453.1, [M + H]⁻; ¹H NMR (400 MHz, DMSO) δ 11.31 |
| | (s, 1H), 8.53 (s, 2H), 7.64 (d, J = 7.4 Hz, 1H), 7.30 (d, J = 10.2 |
| | Hz, 1H), 7.18 (q, J = 8.4 Hz, 4H), 7.01 (s, 1H), 4.77 (d, J = 13.0 |
| | Hz, 1H), 4.46 (t, J = 5.1 Hz, 1H), 3.42-3.35 (m, 2H), 3.23-3.14 |
| | (m, 1H), 2.84-2.76 (m, 2H), 2.59 (s, 2H), 1.71-1.62 (m, 2H). |
| 166 | MS m/z = 477.1, [M + H]⁺; ¹H NMR (400 MHz, DMSO) δ 11.31 |
| | (s, 1H), 8.78 (s, 1H), 7.67 (d, J = 7.4 Hz, 1H), 7.47-7.18 (m, 5H), |
| | 7.10 (d, J = 4.9 Hz, 2H), 5.16 (t, J = 5.7 Hz, 1H), 4.91 (s, 1H), |
| | 4.46 (d, J = 5.7 Hz, 2H), 3.30-3.20 (m, 1H), 2.93-2.73 (m, 2H). |

### Example 31

### Preparation of Compound 399

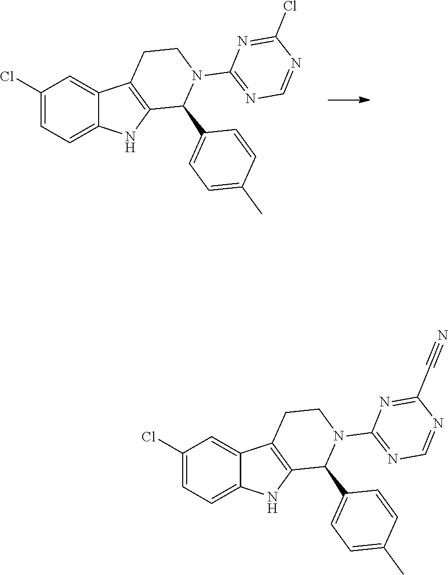 [SMILES: Cc1ccc([C@H]2c3[nH]c4ccc(Cl)cc4c3CCN2c2ncnc(Cl)n2)cc1 ; Cc1ccc([C@H]2c3[nH]c4ccc(Cl)cc4c3CCN2c2ncnc(C#N)n2)cc1]

To a RBF, was added: (1S)-6-chloro-2-(4-chloro-1,3,5-triazin-2-yl)-1-(p-tolyl)-1,3,4,9-tetrahydropyrido[3,4-b]indole (150 mg, 0.3656 mmol), tetraethylammonium cyanide (150 mg, 0.902279 mmol) in acetonitrile (15 mL, 286 mmol), the mixture was stirred at RT overnight. LCMS showed the desired product as the major peak. The mixture was purified through Prep-HPLC, then Prep-TLC to get Compound 399 (20 mg, 0.04989 mmol, 100 13.65% yield) as a pale yellow solid. MS m/z=401.0, [M+H]⁺; (DMSO-d, 400 MHz) 11.26 (d, J=12.4 Hz, 1H), 8.84 (d, J=22.8 Hz, 1H), 7.55 (s, 1H), 7.33 (q, J=4.0 Hz, 1H), 7.18 (d, J=2.0 Hz, 4H), 7.10 (t, J=1.6 Hz, 1.5H), 6.97 (s, 0.5H), 4.81 (m, 1H), 3.25 (m, 1H), 2.87 (m, 2H), 2.28 (d, J=2.0 Hz, 3H).

Using the procedure described for Compound 399 above, additional compounds described herein were prepared by substituting the appropriate starting materials, suitable reagents, and reaction conditions, obtaining compounds such as those selected from:

| Cpd | Data |
|---|---|
| 411 | MS m/z = 413.0, [M + H]⁺; ¹H NMR (400 MHz, DMSO) δ 11.26 (s, 1H), 7.54 (s, 1H), |
| | 7.32 (d, J = 4.8 Hz, 1H), 6.98-7.18 (m, 6H), 4.83 (q, J = 9.2 Hz, 1H), 3.24 (d, J = 10.8 |
| | Hz, 1H), 2.82-2.93 (m, 2H), 2.43 (s, 3H), 2.28 (s, 3H) |
| 414 | MS m/z = 474.0, [M + H]⁺; ¹H NMR (400 MHz, CDCL3) δ 11.13 (d, J = 16.4 Hz, 1H), |
| | 8.84 (d, J = 19.2 Hz, 1H), 8.32 (d, J = 4.0 Hz, 2H), 7.57 (s, 1H), 7.32-7.36 (m, 1H), 7.10 |
| | (q, J = 2.0 Hz, 1H), 6.83 (d, J = 34.8 Hz, 1H), 4.84-4.97 (m, 1H), 3.67 (t, J = 4.6 Hz, 4H), |
| | 3.62 (d, J = 2.4 Hz, 4H), 3.41-3.45 (m, 1H), 2.97 (d, J = 15.6 Hz, 1H), 2.78-2.85 (m, |
| | 1H). |
| 415 | MS m/z = 487.9, [M + H]⁺; ¹H NMR (400 MHz, CDCL3) δ 11.12 (d, J = 11.2 Hz, 1H), |
| | 8.32 (t, J = 6.6 Hz,2H), 7.56 (s, 1H), 7.33 (q, J = 5.4 Hz, 1H), 7.10 (d, J = 8.4 Hz, 1H), |
| | 6.84 (d, J = 46.4 Hz, 1H), 4.84-5.04 (m, 1H), 3.63 (d, J = 4.4 Hz, 4H), 3.49 (d, J = 12.8 |
| | Hz, 4H), 3.35-3.47 (m, 1H), 2.97 (d, J = 14.8 Hz, 1H) 2.79-2.82 (m, 1H), 2.44 (s, 3H). |

### Example 32

### Preparation of Compound 416

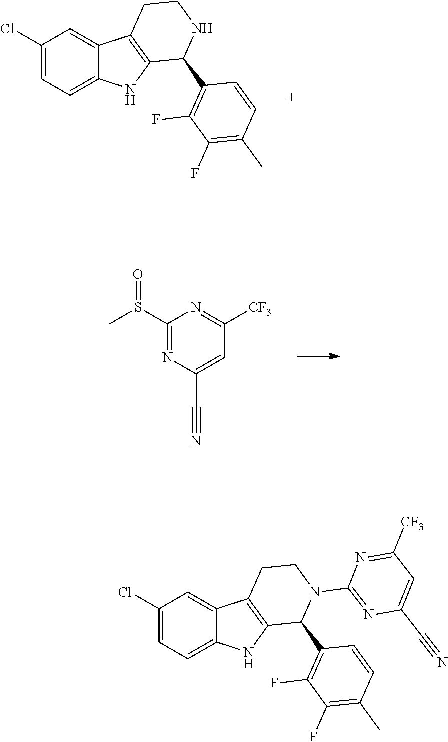 [SMILES: Cc1ccc([C@@H]2NCCc3c2[nH]c2ccc(Cl)cc32)c(F)c1F ; CS(=O)c1nc(C#N)cc(C(F)(F)F)n1 ; Cc1ccc([C@H]2c3[nH]c4ccc(Cl)cc4c3CCN2c2nc(C#N)cc(C(F)(F)F)n2)c(F)c1F]

To a 3-neck RBF, equipped with a N2 inlet, was added: 2-(5-bromo-1H-indol-3-yl)ethanamine (150 mg, 0.4507 mmol), N,N-Diisopropylamine (0.25 mL, 1.4 mmol) in tetrahydrofuran (2 mL, 24.6 mmol) and treated with 2-methylsulfinyl-6-(trifluoromethyl)pyrimidine-4-carbonitrile (300 mg, 1.2756 mmol). Stir reaction at RT for 3 hours. Concentrated and purified via prep-HPLC to get Compound 416 (20 mg, 0.03970 mmol, 8.808% yield) as a white solid. MS m/z: =401.0, [M−H]⁻; (DMSO-d, 400 MHz) 11.18 (s, 1H), 7.81 (s, 1H), 7.57 (s, 1H), 7.32 (t, J=2.8 Hz, 1H), 7.16 (d, J=2.0 Hz, 4H), 7.09 (d, J=6.4 Hz, 1H), 7.05 (s, 1H), 6.80 (s, 1H), 4.83 (d, J=15.6 Hz, 1H), 3.49 (s, 1H), 2.99-2.85 (m, 2H), 2.26 (s, 3H).

Using the procedure described for Compound 416 above, additional compounds described herein were prepared by substituting the appropriate starting materials, suitable reagents, and reaction conditions, obtaining compounds such as those selected from:

| Cpd | Data |
|---|---|
| 425 | MS m/z = 466.0 [M − H]⁻; ¹H NMR (400 MHz, CDCl3) δ 11.21 (d, J = 9.2 Hz, 1H), 7.77 |
| | (s, 1H), 7.55 (s, 1H), 7.33 (t, J = 8.8 Hz, 1H), 7.22-7.16 (m, 4H), 7.09 (q, J = 6.4 Hz, |
| | 1H), 6.97 (d, J = 7.6 Hz, 1H), 4.18 (s, 1H), 3.32 (s, 1H), 2.92-2.86 (m, 2H), 2.28 |
| | (s,3H). |
| 433 | MS m/z = 538.0 [M − H]⁻; ¹H NMR (DMSO-d, 400 MHz) δ 11.13 (s, 1H), 8.13 (d, J = 2.0 |
| | Hz, 1H), 7.77 (s, 1H), 7.56 (s, 1H), 7.44 (t, J = 6.8 Hz, 1H), 7.32 (t, J = 7.2 Hz, 1H), 7.09 |
| | (q, J = 6.4 Hz, 1H), 6.83 (q, J = 10.0 Hz, 2H), 4.88 (d, J = 14.8 Hz, 1H), 3.66 (d, J = 4.0 |
| | Hz, 4H), 3.41 (s, 4H), 3.31 (s, 1H), 2.96 (d, J = 14.0 Hz, 1H), 2.85 (s, 1H). |
| 434 | MS m/z = 539.0 [M − H]⁻; ¹H NMR (DMSO-d, 400 MHz) δ 11.09 (s, 1H), 8.33 (s, 2H), |
| | 7.79 (s, 1H), 7.57 (s, 1H), 7.32 (d, J = 8.4 Hz, 1H), 7.09 (q, J = 6.8 Hz, 1H), 6.74(d, |
| | J = 14.4 Hz, 1H), 4.92 (d, J = 12 Hz, 1H), 3.67 (s, 4H), 3.62 (d, J = 4.8 Hz, 4H), 3.52- |
| | 3.43 (m, 1H), 3.00 (d, J = 15.2 Hz, 1H), 2.82 (d, J = 10.8 Hz, 1H). |

### Example 33

### Preparation of Compound 426

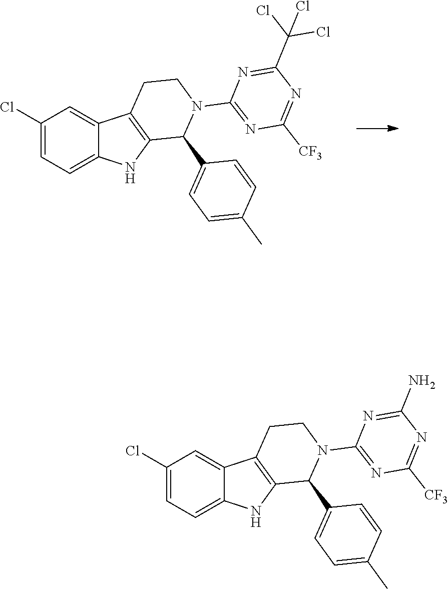 [SMILES: Cc1ccc([C@H]2c3[nH]c4ccc(Cl)cc4c3CCN2c2nc(C(F)(F)F)nc(C(Cl)(Cl)Cl)n2)cc1 ; Cc1ccc([C@H]2c3[nH]c4ccc(Cl)cc4c3CCN2c2nc(N)nc(C(F)(F)F)n2)cc1]

(1S)-6-chloro-1-(p-tolyl)-2-[4-(trichloromethyl)-6-(trifluoromethyl)-1,3,5-triazin-2-yl]-1,3,4,9-tetrahydropyrido[3,4-b]indole (80 mg, 0.1426 mmol) was mixed with ammonia (2 mol/L) in methanol (3 mL, 21 mmol, 7 mol/L). The mixture was stirred overnight at 40° C. in a sealed tube. Then the mixture was concentrated and purified by prep-HPLC to provide Compound 426 (49 mg, 0.1068 mmol, 74.90%/yield) as a white solid. MS m/z=458.9, [M+H]⁺; (DMSO-d, 400 MHz) δ 11.24 (d, J=24.8 Hz, 1H), 7.61-7.75 (m, 2H), 7.53 (d, J=2.0 Hz, 1H), 7.31 (dd, J=8.6 Hz, 1H), 7.24 (d, J=8.0 Hz, 1H), 7.16-7.17 (m, 3H), 7.08 (dd, 0.1-8.6 Hz, 1H), 7.03 (d, 0.1-11.2 Hz, 1H), 4.79-4.87 (m, 1H), 3.10-3.18 (m, 1H), 2.72-2.88 (m, 2H), 2.27 (d, J=2.8 Hz, 3H).

Using the procedure described for Compound 426 above, additional compounds described herein were prepared by substituting the appropriate starting materials, suitable reagents, and reaction conditions, obtaining compounds such as those selected from:

| Cpd | Data |
|---|---|
| 427 | MS m/z = 522.9, [M + H]⁺; ¹H NMR (DMSO-d, 400 MHz) δ 11.19 (d, J = 29.6 Hz, 1H), |
| | 7.77 (d, J = 10.4 Hz, 1H), 7.63 (d, J = 10.8 Hz, 1H), 7.54 (s, 1H), 7.32 (t, J = 7.6 Hz, 1H), |
| | 7.23 (d, J = 6.4 Hz, 1H), 7.01-7.10 (m, 2H), 6.74-6.82 (m, 1H), 4.85-4.91 (m, 1H), |
| | 3.25-3.39 (m, 1H), 2.82-2.87 (m, 2H), 2.26(s, 3H). |
| 435 | MS m/z = 494.9, [M + H]⁺; ¹H NMR (400 MHz, DMSO) δ 11.11 (d, J = 28.8 Hz, 1H), |
| | 7.54 (s, 1H), 7.30 (d, J = 8.4 Hz, 1H), 7.24 (s, 1H), 7.06 (q, J = 20.4 Hz, 2H), 6.74-6.87 |
| | (m, 1H), 4.93 (q, J = 12.0 Hz, 1H), 3.42-3.49 (m, 1H), 3.13-3.18 (m, 6H), 2.73-2.94 (m, |
| | 2H), 2.26 (s, 3H). |
| 436 | MS m/z = 564.9, [M + H]⁺; ¹H NMR (400 MHz, DMSO) δ 11.10 (d, J = 36.4 Hz, 1H), |
| | 7.55 (d, J = 2.0 Hz, 1H), 7.31 (d, J = 8.8 Hz, 1H), 7.23 (s, 1H), 7.04-7.10 (m, 2H), 6.73- |
| | 6.87 (m, 1H), 4.91 (q, J = 12.0 Hz, 1H), 3.64-3.92 (m, 8H), 3.41-3.45 (m, 1H), 2.73- |
| | 2.95 (m, 2H), 2.26 (s, 3H). |
| 441 | MS m/z = 586.9, [M + H]⁺; ¹H NMR (400 MHz, DMSO) δ 11.19 (d, J = 23.6 Hz, 1H), |
| | 7.52-7.54 (m, 1H), 7.31 (d, J = 8.8 Hz, 1H), 7.23 (d, J = 8.0 Hz, 1H), 7.15-7.18 (m, |
| | 3H), 7.03-7.10 (m, 2H), 4.83-4.92 (m, 1H), 3.10-3.26 (m, 7H), 2.75-2.90 (m, 2H), |
| | 2.27 (d, J = 2.8 Hz, 3H). |
| 442 | MS m/z = 528.9, [M + H]⁺; ¹H NMR (400 MHz, DMSO) δ 11.11-11.29 (m, 1H), 7.52- |
| | 7.57 (m, 1H), 7.31 (dd, J = 8.8 Hz, 1H), 7.16-7.21 (m, 4H), 7.02-7.09 (m, 2H), 4.82- |
| | 4.90 (m, 1H), 3.63-3.99 (m, 8H), 3.12-3.27 (m, 1H), 2.72-2.91 (m, 2H), 2.27 (d, J = |
| | 2.4 Hz, 3H). |
| 443 | MS m/z = 558.9, [M + H]⁺; ¹H NMR (400 MHz, DMSO) δ 11.12 (d, J = 17.6 Hz, 1H), |
| | 8.12 (d, J = 41.6 Hz, 1H), 7.54 (s, 1H), 7.39-7.46 (m, 1H), 7.31 (d, J = 8.8 Hz, 1H), 7.08 |
| | (d, J = 8.8 Hz, 1H), 6.96 (d, J = 24.8 Hz, 1H), 6.80 (d, J = 8.8 Hz, 1H), 4.89-4.97 (m, 1H), |
| | 3.66 (s, 4H), 3.40 (s, 4H), 3.13-3.28 (m, 7H), 2.89-2.93 (m, 1H), 2.76-2.83 (m, 1H). |
| 444 | MS m/z = 559.9, [M + H]⁺; ¹H NMR (400 MHz, DMSO) δ 11.06 (d, J = 10.0 Hz, 1H), |
| | 8.36 (s, 1H), 8.28 (s, 1H), 7.55 (dd, J = 5.4, 2.0 Hz, 1H), 7.32 (d, J = 8.4 Hz, 1H), 7.10- |
| | 7.07 (m, 1H), 6.85 (d, J = 33.6 Hz, 1H), 5.02-4.93 (m, 1H), 3.67-3.62 (m, 8H), 3.36- |
| | 3.25 (m, 3H), 3.16-3.14 (m, 4H), 2.96-2.90 (m, 1H), 2.82-2.68 (m, 1H). |
| 445 | MS m/z = 602.0, [M + H]⁺; ¹H NMR (400 MHz, DMSO) δ 11.04 (d, J = 22.8 Hz, 1H), |
| | 8.31 (d, J = 1.6 Hz, 2H), 7.55 (dd, J = 6.0, 2.0 Hz, 1H), 7.32 (dd, J = 8.4, 1.6 Hz, 1H), |
| | 7.10-7.07 (m, 1H), 6.88 (d, J = 40.8 Hz, 1H), 5.00-4.92 (m, 1H), 4.07-3.89 (m, 1H), |
| | 3.77-3.72 (m, 3H), 3.67-3.61 (m, 12H), 3.38-3.27 (m, 1H), 2.96-2.90 (m, 1H), 2.82- |
| | 2.74 (m, 1H). |
| 446 | MS m/z = 601.0, [M + H]⁺; ¹H NMR (400 MHz, DMSO) δ 11.10 (d, J = 32.4 Hz, 1H), |
| | 8.07-8.13 (m, 1H), 7.54 (d, J = 3.2 Hz, 1H), 7.39-7.44 (m, 1H), 7.30-7.32 (m, 1H), 7.08 |
| | (d, J = 8.4 Hz, 1H), 6.96 (d, J = 23.2 Hz, 1H), 6.80 (d, J = 8.8 Hz, 1H), 4.91-4.92 (m, 1H), |
| | 3.66-4.04 (m, 13H), 3.40 (s, 3H), 3.22-3.30 (m, 1H), 2.90-2.93 (m, 1H), 2.76-2.79 (m, |
| | 1H). |
| 509 | MS m/z = 615.2, [M + H]⁺; ¹H NMR (400 MHz, DMSO) δ 11.03 (d, J = 23.5 Hz, 1H), |
| | 8.27 (d, J = 19.7 Hz, 2H), 7.55 (dd, J = 5.7, 1.9 Hz, 1H), 7.31 (dd, J = 8.6, 1.6 Hz, |
| | 1H), 7.08 (d, J = 8.6 Hz, 1H), 6.85 (d, J = 39.3 Hz, 1H), 4.96 (dd, J = 13.1, 8.5 Hz, |
| | 1H), 3.96 (d, J = 65.4 Hz, 1H), 3.73 (d, J = 27.6 Hz, 11H), 3.29-3.24 (m, 1H), 2.93 (d, |
| | J = 15.6 Hz, 1H), 2.84-2.65 (m, 1H), 2.31 (s, 4H), 2.18 (s, 3H). |

### Example 34

### Preparation of Compound 390

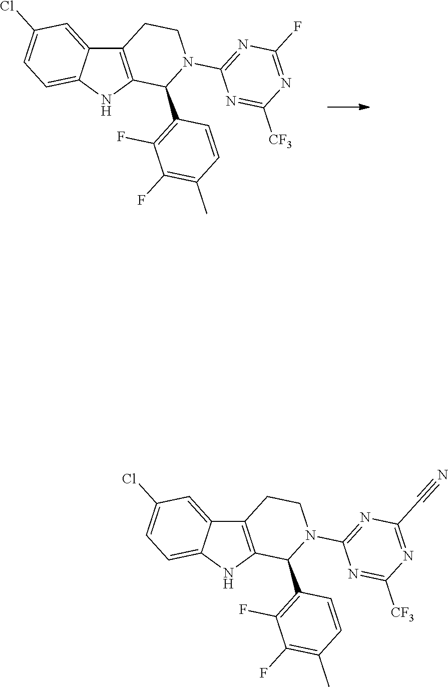 [SMILES: Cc1ccc([C@H]2c3[nH]c4ccc(Cl)cc4c3CCN2c2nc(F)nc(C(F)(F)F)n2)c(F)c1F ; Cc1ccc([C@H]2c3[nH]c4ccc(Cl)cc4c3CCN2c2nc(C#N)nc(C(F)(F)F)n2)c(F)c1F]

To a screw-cap vial was added (1S)-6-chloro-1-(2,3-difluoro-4-methyl-phenyl)-2-[4-fluoro-6-(trifluoromethyl)-1,3,5-triazin-2-yl]-1,3,4,9-tetrahydropyrido[3,4-b]indole (50 mg, 0.10 mmol) and DMF (2 mL) followed by potassium cyanide (13 mg, 0.20 mmol). The reaction mixture was stirred at RT for 12 Hours. Organics were precipitated out of solution by addition of 10 mL of water. The solids were collected by filtration and purified by silica gel chromatography to afford Compound 390 (38 mg, 0.075 mmol, 75% yield) as a white solid. MS m/z=479.0, [M+H]⁺; ¹H NMR (400 MHz, DMSO-d₆) δ 11.17 (s, 1H), 9.26 (s, 1H), 7.57 (d, J=2.1 Hz, 1H), 7.34 (d, J=8.6 Hz, 1H), 7.19-6.99 (m, 3H), 6.86-6.67 (m, 1H), 5.09 (s, 1H), 3.54 (s, 1H), 2.99 (d, J=15.8 Hz, 2H), 2.26 (s, 3H).

### Example 35

### Preparation of Compound 474

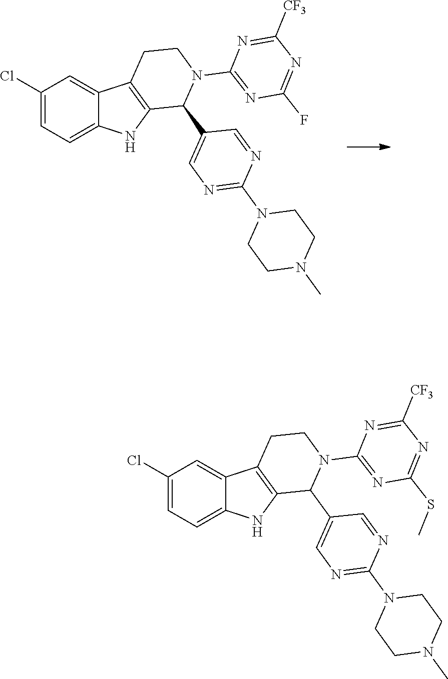 [SMILES: CN1CCN(c2ncc([C@H]3c4[nH]c5ccc(Cl)cc5c4CCN3c3nc(F)nc(C(F)(F)F)n3)cn2)CC1 ; CSc1nc(N2CCc3c([nH]c4ccc(Cl)cc34)C2c2cnc(N3CCN(C)CC3)nc2)nc(C(F)(F)F)n1]

To a 3-neck RBF, equipped with a N2 inlet, added: (1S)-6-chloro-2-[4-fluoro-6-(trifluoromethyl)-1,3,5-triazin-2-yl]-1-[2-(4-methylpiperazin-1-yl)pyrimidin-5-yl]-1,3,4,9-tetrahydropyrido[3,4-b]indole (150 mg, 0.2738 mmol), sodium thiomethoxide (30 mg, 0.4066 mmol), dichloromethane (2 mL, 31.20 mmol). Stir reaction at rt for 1 h. Filter off the solid and concentrate the filtrate to get Compound 474 (100 mg, 0.1736 mmol, 63.41% yield) as crude. 50 mg crude purify via prep-HPLC to get 10 mg pure. MS m/z=576.0, [M+H]⁺; (DMSO-d, 400 MHz) 11.10 (s, 1H), 8.30 (d, J=12.4 Hz, 2H), 7.57 (q, J=2.8 Hz, 1H), 7.33 (q, J=6.4, 1H), 7.10 (q, J=6.8 Hz, 1H), 6.88 (d, J=72.8 Hz, 1H), 5.08-4.90 (m, 1H), 3.71 (s, 4H), 3.47-3.40 (m, 1H), 2.98 (q, J=8.0 Hz, 1H), 2.88-2.80 (m, 1H), 2.60 (d, J=32.8 Hz, 3H), 2.32 (s, 4H). 2.19 (s, 3H).

Using the procedure described for Compound 474 above, additional compounds described herein were prepared by substituting the appropriate starting materials, suitable reagents, and reaction conditions, obtaining compounds such as those selected from:

| Cpd | Data |
|---|---|
| 389 | MS m/z = 526.0, [M + H]⁺; ¹H NMR (400 MHz, DMSO-d₆) δ 11.16 (d, J = 19.9 Hz, |
| | 1H), 7.59 (t, J = 2.3 Hz, 1H), 7.38-7.19 (m, 2H), 7.15-7.02 (m, 2H), 6.89-6.77 |
| | (m, 1H), 5.14-4.70 (m, 1H), 3.47 (dtd, J = 13.3, 11.4, 4.5 Hz, 1H), 3.02-2.66 (m, |
| | 2H), 2.59 (d, J = 12.3 Hz, 3H), 2.29 (dd, J = 4.1, 2.0 Hz, 3H). |
| 489 | ESI-MS m/z = 572.8 [M − 1]⁻; ¹H NMR (400 MHz, DMSO) δ 11.14 (d, J = 6.7 Hz, 1H), |
| | 8.32-8.00 (m, 1H), 7.56 (s, 1H), 7.49-7.36 (m, 1H), 7.32 (dd, J = 8.6, 2.2 Hz, 1H), |
| | 7.09 (d, J = 8.6 Hz, 1H), 7.06 (s, 1H), 6.81 (d, J = 8.8 Hz, 1H), 5.14-4.74 (m, 1H), |
| | 3.47 (s, 4H), 3.13-2.73 (m, 3H), 2.60 (d, J = 38.0 Hz, 3H), 2.40 (s, 4H), 2.23 (s, 3H). |
| 495 | MS m/z = 510.0, [M + H]⁺; ¹H NMR (400 MHz, DMSO) δ 11.04(d, J = 12.8 Hz, 1H), |
| | 7.35-7.20(m, 3H), 7.06(q, J = 7.2 Hz, 1H), 6.97-6.92 (m, 1H), 6.85-6.77(m, 1H), 5.03- |
| | 4.87 (m, 1H), 3.47-3.32 (m, 1H), 2.97-2.85(m, 2H), 2.57(d, J = 11.6 Hz, 3H), 2.27(d, |
| | J = 2 Hz, 3H). |

### Example 36

### Preparation of Compound 460

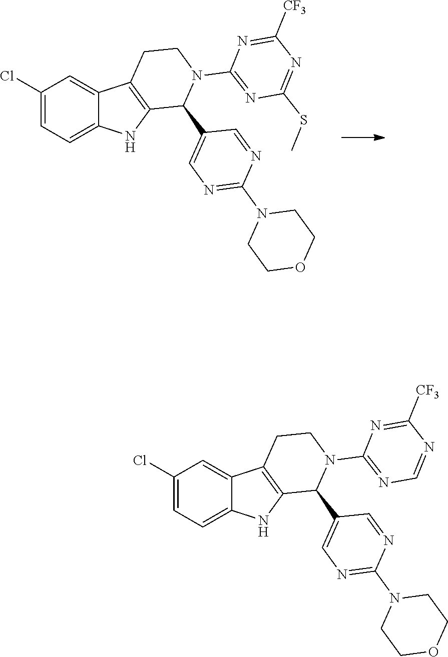 [SMILES: CSc1nc(N2CCc3c([nH]c4ccc(Cl)cc34)[C@@H]2c2cnc(N3CCOCC3)nc2)nc(C(F)(F)F)n1 ; FC(F)(F)c1ncnc(N2CCc3c([nH]c4ccc(Cl)cc34)[C@@H]2c2cnc(N3CCOCC3)nc2)n1]

To a 3-neck RBF, equipped with a N₂ inlet, was added: 4-[5-[(1S)-6-chloro-2-[4-methylsulfanyl-6-(trifluoromethyl)-1,3,5-triazin-2-yl]-1,3,4,9-tetrahydropyrido[3,4-b]indol-1-yl]pyrimidin-2-yl]morpholine (90 mg, 0.1599 mmol), and Raney Nickel (90 mg, 1.5334 mmol) suspended in ethanol (4 mL, 68.7 mmol). The reaction was stirred at 50° C. overnight. The product was collected by filtration, washing twice with ethanol (1 mL), then dried in a vacuum oven. The residue was purified by prep-HPLC to obtain Compound 460 (45 mg, 0.08706 mmol, 54.46% yield) as a white solid. MS m/z=517.0, [M+H]⁺; (DMSO-d₆, 400 MHz) 11.15-11.01 (m, 1H), 8.96-8.90 (m, 1H), 8.35 (d, J=5.6 Hz, 2H), 7.57 (s, 1H), 7.35-7.31 (m, 1H), 7.10 (d, J=8.8 Hz, 1H), 6.93-6.80 (m, 1H), 5.07-4.92 (m, 1H), 3.69-3.62 (m, 8H), 3.51-3.44 (m, 1H), 3.02-2.96 (m, 1H), 2.87-2.79 (m, 1H).

Using the procedure described for Compound 460 above, additional compounds described herein were prepared by substituting the appropriate starting materials, suitable reagents, and reaction conditions, obtaining compounds such as those selected from:

| Cpd | Data |
|---|---|
| 418 | MS m/z = 442.2, [M − H]⁻; ¹H NMR (400 MHz, DMSO-d₆) δ 11.27 (d, J = 17.9 Hz, |
| | 1H), 8.95 (d, J = 24.8 Hz, 1H), 7.57 (t, J = 2.6 Hz, 1H), 7.35 (dd, J = 8.6, 5.4 Hz, |
| | 1H), 7.25-7.18 (m, 4H), 7.17-7.08 (m, 2H), 7.04 (s, 1H), 4.92 (ddd, J = 45.0, |
| | 13.4, 5.3 Hz, 1H), 3.03-2.69 (m, 2H), 2.30 (s, 3H). |
| 488 | MS m/z = 530.0, [M + H]⁺: ¹H NMR (DMSO-d, 400 MHz) δ 11.13 (d, J = 20.0 Hz, |
| | 1H), 8.94 (d, J = 20.0 Hz, 1H), 8.33-8.26 (m, 2H), 7.57 (t, J = 2.4 Hz, 1H), 4.33 (q, |
| | J = 5.6 Hz, 1H), 7.12-7.09 (m, 1H), 6.85 (d, J = 51.2 Hz, 1H), 5.06-4.91 (m, 1H), 3.71 (t, |
| | J = 5.2 Hz. 4H), 3.50-3.41 (m, 1H), 3.00 (d, J = 14.0 Hz, 1H), 2.87-2.79 (m, 1H), 2.33 |
| | (t, J = 4.8 Hz, 4H), 2.19 (d, J = 4.4 Hz, 3H). |
| 491 | MS m/z = 529.0, [M + H]⁺; ¹H NMR (DMSO-d₆, 400 MHz) δ 11.20-11.56 (m, 1H), |
| | 8.97-8.90 (m, 1H), 8.11 (d, J = 2.4 Hz, 1H), 7.56 (t, J = 2.4 Hz & J = 4.8 Hz, 1H), 7.43- |
| | 7.40 (m, 1H), 7.34-7.30 (m, 1H), 7.10 (d, J = 8.4 Hz, 1H), 7.02-6.90 (m, 1H), 6.83- |
| | 6.79 (m, 1H), 5.02-4.87 (m, 1H), 3.47 (t, J = 4.4 Hz & J = 9.2 Hz, 4H), 3.39-3.30 (s, |
| | 1H), 3.04-2.96 (m, 1H), 2.88-2.80 (m, 1H), 2.40 (t, J = 4.4 Hz & J = 9.2 Hz, 4H), 2.22 |
| | (s, 3H). |
| 494 | MS m/z = 464.0, [M + H]⁺; ¹H NMR (400 MHz, DMSO) δ 11.08(s, 1H), 8.95(d, J = |
| | 14 Hz, 1H), 7.34-7.22(m, 3H), 7.05 (q, J = 7.6 Hz, 1H), 6.97-6.92(m, 1H), 6.80 (q, |
| | J = 4 Hz, 1H), 5.02-4.86 (m, 1H), 3.50-3.31 (m, 1H), 2.98-2.84(m, 2H), 2.27(s, 3H). |

### Example 37

### Preparation of Compound 464

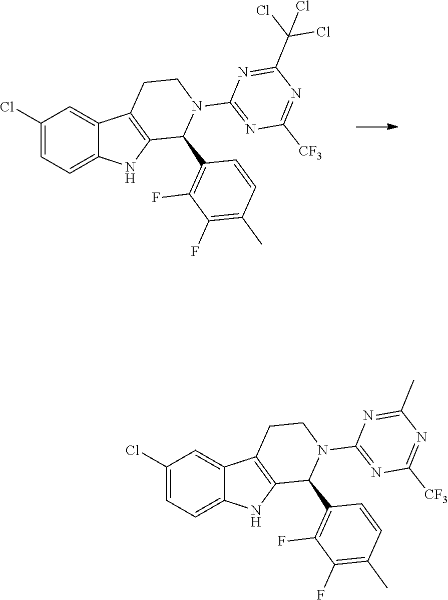 [SMILES: Cc1ccc([C@H]2c3[nH]c4ccc(Cl)cc4c3CCN2c2nc(C(F)(F)F)nc(C(Cl)(Cl)Cl)n2)c(F)c1F ; Cc1nc(N2CCc3c([nH]c4ccc(Cl)cc34)[C@@H]2c2ccc(C)c(F)c2F)nc(C(F)(F)F)n1]

In a 50.0 mL single neck round-bottomed flask to a solution of (1S)-6-chloro-1-(2,3-difluoro-4-methyl-phenyl)-2-[4-(trichloromethyl)-6-(trifluoromethyl)-1,3,5-triazin-2-yl]-1,3,4,9-tetrahydropyrido[3,4-b]indole (100 mg, 0.1674 mmol) in a mixture of ethanol (6.0 mL, 100 mmol) and water (2.0 mL, 110 mmol) was added ammonium chloride (90.0 mg, 1.68 mmol) and zinc powder (110 mg, 1.682 mmol). The mixture was stirred at RT for 2 hr, until LCMS showed complete conversion to the desired product. The mixture was filtered and the filtrate was concentrated to give a residue, which was purified by Prep-H-PLC to provide Compound 464 as a yellow solid. MS m/z=493.9, [M+H]⁺; ¹H NMR (400 MHz, CDCl3) δ 7.95 (d, J=10.8 Hz, 1H), 7.50 (s, 1H), 7.21-7.34 (m, 2H), 7.15 (d, J=18.4 Hz, 1H), 6.77-6.85 (m, 2H), 5.20-5.28 (m, 1H), 3.46-3.52 (m, 1H), 2.88-2.97 (m, 2H), 2.55 (d, J=8.0 Hz, 3H), 2.29 (s, 3H).

Using the procedure described for Compound 464 above, additional compounds described herein were prepared by substituting the appropriate starting materials, suitable reagents, and reaction conditions, obtaining compounds such as those selected from:

| Cpd | Data |
|---|---|
| 465 | MS m/z = 529.9, [M + H]⁺; ¹H NMR (400 MHz, DMSO) δ 11.17 (d, J = 11.2 Hz, 1H), |
| | 8.13 (d, J = 12.4 Hz, 1H), 7.55 (s, 1H), 7.44-7.50 (m, 1H), 7.32 (d, J = 3.2 Hz, 1H), 6.91- |
| | 7.11 (m, 2H), 6.80 (d, J = 8.0 Hz, 1H), 4.88-5.04 (m, 1H), 3.66-3.86 (m, 8H), 3.18 (s, |
| | 1H), 2.82-2.98 (m, 2H), 2.42-2.56 (m, 3H). |
| 466 | MS m/z = 530.9, [M + H]⁺; ¹H NMR (400 MHz, DMSO) δ 11.10 (d, J = 16.4 Hz, 1H), |
| | 8.34 (d, J = 14.8 Hz, 2H), 7.56 (t, J = 1.8 Hz, 1H), 7.33 (dd, J = 8.6, 1.6 Hz, 1H), 7.10 |
| | (dd, J = 8.4, 2.0 Hz, 1H), 6.87 (J = 60.8 Hz, 1H), 5.10-4.91 (m, 1H), 3.68-3.62 (m, |
| | 8H), 3.46-3.42 (m, 1H), 3.01-2.95 (m, 1H), 2.84-2.78 (m, 1H), 2.52 (d, J = 27.6 Hz, |
| | 3H). |
| 467 | MS m/z = 457.9, [M + H]⁺; ¹H NMR (400 MHz, DMSO) δ 11.24 (d, J = 12.8 Hz, 1H), |
| | 7.54 (t, J = 2.0 Hz, 1H), 7.32 (dd, J = 8.6, 4.8 Hz, 1H), 7.22-7.17 (m, 4.5H), 7.09 (dd, |
| | J = 8.6, 1.6 Hz, 1H), 7.03 (s, 0.5H), 5.01-4.81 (m, 1H), 3.32-3.24 (m, 2H), 2.97-2.80 (m, |
| | 2H), 2.52 (d, J = 27.6 Hz, 3H), 2.28(d, J = 2.4 Hz, 3H), |

### Example 38

### Preparation of Compound 471

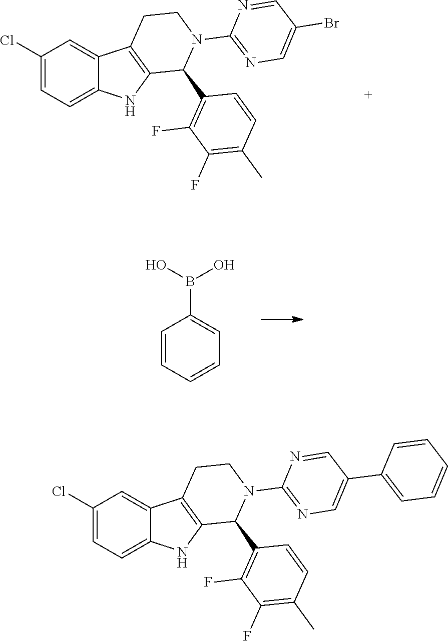 [SMILES: Cc1ccc([C@H]2c3[nH]c4ccc(Cl)cc4c3CCN2c2ncc(Br)cn2)c(F)c1F ; OB(O)c1ccccc1 ; Cc1ccc([C@H]2c3[nH]c4ccc(Cl)cc4c3CCN2c2ncc(-c3ccccc3)cn2)c(F)c1F]

To a 100 mL single neck flask with N₂-inlet was added (1S)-2-(5-bromopyrimidin-2-yl)-6-chloro-1-(2,3-difluoro-4-methyl-phenyl)-1,3,4,9-tetrahydropyrido[3,4-b]indole (150 mg, 0.3063 mmol), phenylboronic acid (41 mg, 0.33626 mmol), chloro(2-dicyclohexylphosphino-2′,6′-diisopropoxy-1,1′-biphenyl)[2-(2′-amino-1,1′-biphenyl)]palladium(II) (0.1 mmol) potassium carbonate (84 mg, 0.607797 mmol), water (3 mL, 166.530 mmol) in 1,4-dioxane (3 mL, 35.14 mmol). The mixture was stirred under nitrogen at 90° C. overnight. Analysis by LCMS showed conversion to the desired mass. Reaction mixture was extracted with DCM, the organics were washed by water and dried over Na2SO4. The organics were then concentrated and purified by prep-HPLC to give the desired product as white solid, Compound 471 (66 mg, 0.1356 mmol, 44.25% yield) MS m/z=487.0, [M+H]⁺; (CDCl3, 400 MHz), δ: 8.59 (s, 2H), 8.07 (s, 1H), 7.50-7.41 (m, 5H), 7.35-7.32 (m, 2H), 7.24-7.22 (m, 1H), 7.13-7.10 (m, 1H), 6.84-6.82 (m, 2H), 5.24-5.20 (m, 1H), 3.55-3.49 (m, 1H), 2.99 (m, 1H), 2.28 (m, 3H).

Using the procedure described for Compound 471 above, additional compounds described herein were prepared by substituting the appropriate starting materials, suitable reagents, and reaction conditions, obtaining compounds such as those selected from:

| Cpd | Data |
|---|---|
| 472 | MS m/z = 525.0, [M + H]⁺; ¹H NMR (CDCl₃, 400 MHz), δ |
| | 8.59(s, 2H), 8.40(s, 2H), 7.88(s, 1H), 7.52-7.43(m, 5H), 7.37- |
| | 7.33(m, 1H), 7.20-7.18(m, 1H), 7.14-7.11(m, 1H), 7.05(s, 1H), |
| | 5.15-5.10(m, 1H), 3.76-3.72(m, 8H), 3.34-3.27(m, 1H), 2.97- |
| | 2.84(m, 2H) |

### Example 39

### Preparation of Compound 476 and 477

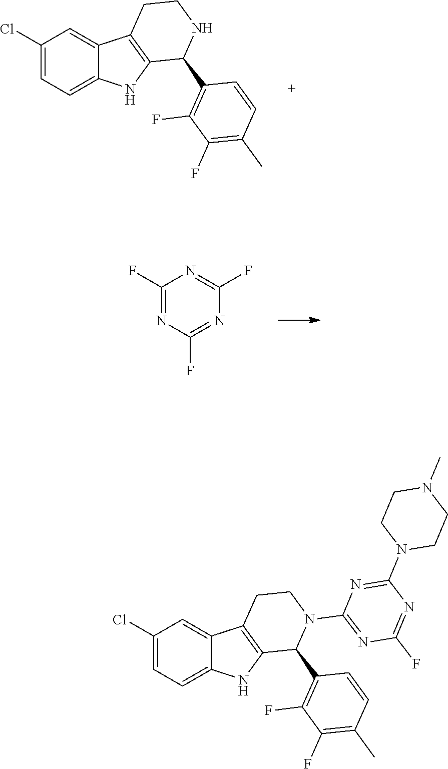 [SMILES: Cc1ccc([C@@H]2NCCc3c2[nH]c2ccc(Cl)cc32)c(F)c1F ; Fc1nc(F)nc(F)n1 ; Cc1ccc([C@H]2c3[nH]c4ccc(Cl)cc4c3CCN2c2nc(F)nc(N3CCN(C)CC3)n2)c(F)c1F]

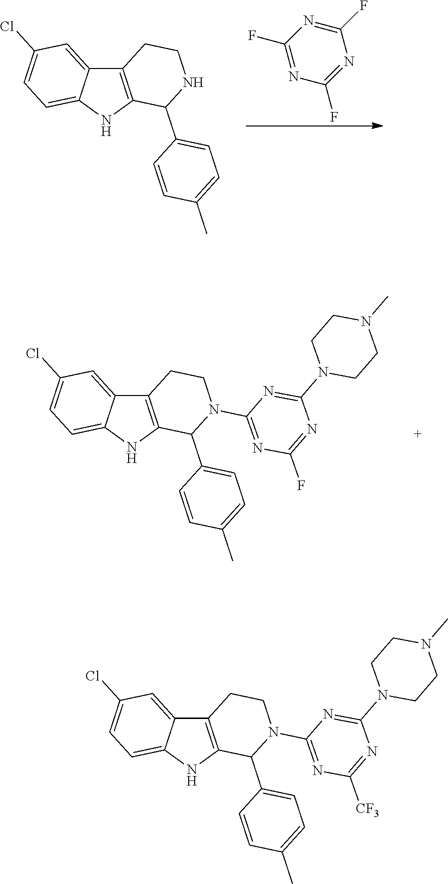 [SMILES: Cc1ccc(C2NCCc3c2[nH]c2ccc(Cl)cc32)cc1 ; Fc1nc(F)nc(F)n1 ; Cc1ccc(C2c3[nH]c4ccc(Cl)cc4c3CCN2c2nc(F)nc(N3CCN(C)CC3)n2)cc1 ; Cc1ccc(C2c3[nH]c4ccc(Cl)cc4c3CCN2c2nc(N3CCN(C)CC3)nc(C(F)(F)F)n2)cc1]

18-Crown-6 (0.32 g, 1.2 mmol) and 4.0 g of 4 A molecular sieves (powder) were mixed in acetonitrile (50.0 mL). The mixture was cooled to 0° C. Then 2,4,6-trifluoro-1,3,5-triazine (2.0 mL, 24 mmol) and (trifluoromethyl)trimethylsilane (8.9 mL, 60 mmol) were added. Cesium fluoride (4.0 g, 26 mmol) was added in several portions over 25 min. After that, the mixture was stirred for 30 min at 0° C. (as monitored by LC-MS (after mini-quench with N-methylpiperazine). (1S)-6-chloro-1-(2,3-difluoro-4-methyl-phenyl)-2,3,4,9-tetrahydro-1H-pyrido[3,4-b]indole (2.0 g, 6.0 mmol) was added in one portion. After that, the mixture was stirred for 1H at RT and quenched with N-methylpiperazine. The mixture was filtered. The filtrate was concentrated and purified by silica gel chromatography to give Compound 476 (54 mg, 5.4% yield) as a white powder. MS m/z=528.1, [M+H]⁺; ¹H NMR (400 MHz, DMSO-d₆) δ 11.8 (d, 1H, J=23.2 Hz), 7.54 (d, 1H, J=2.0 Hz), 7.32 (t, 1H, J=8.2 Hz), 7.23 (s, 0.6H), 7.12-7.02 (m, 2.4H), 6.77 (dt, 1H, J=50.8 Hz, 6.8 Hz), 4.89-4.76 (m, 1H), 3.85-3.66 (m, 4H), 3.42-3.37 (m, 1H), 2.91-2.76 (m, 2H), 2.51-2.34 (m, 4H), 2.26 (s, 3H), 2.22 (s, 3H).

Compound 477 was isolated as a byproduct from the same reaction mixture. MS m/z=578.1, [M+H]⁺; ¹H NMR (400 MHz, DMSO) δ 11.10 (d, J=−37.6 Hz, 1H), 7.55 (s, 1H), 7.31 (d, J=8.4 Hz, 1H), 7.22 (d, J=6.0 Hz, 1H), 7.02-7.10 (m, 2H), 6.75-6.86 (m, 1H), 4.90-4.93 (m, 1H), 3.76-3.88 (m, 4H), 3.39-3.46 (m, 1H), 2.77-2.95 (m, 2H), 2.44-2.51 (m, 4H), 2.26-2.77 (m, 6H).

Using the procedure described for Compound 476 above, additional compounds described herein may be prepared by substituting the appropriate starting materials, suitable reagents, and reaction conditions, obtaining compounds such as those selected from:

| Cpd | Data |
|---|---|
| 478 | MS m/z = 615.2, [M + H]⁺; ¹H NMR (400 MHz, DMSO) δ 11.04 |
| | (d, J = 23.2 Hz, 1H), 8.31 (d, J = 20.8 Hz, 2H), 7.55 (dd, J = |
| | 6.0, 2.0 Hz, 1H), 7.32 (dd, J = 8.4, 2.4 Hz, 1H), 7.09 (dd, J = |
| | 8.6, 2.0 Hz, 1H), 6.85 (d, J = 38.4 Hz, 1H), 5.00-4.92 (m, 1H), |
| | 4.06-3.83 (m, 3H), 3.67-3.61 (m, 8H), 2.96-2.91 (m, 1H), 2.82- |
| | 2.74 (m, 1H), 2.29 (d, J = 4.4 Hz, 3H). |
| 479 | MS m/z = 628.2, [M + H]⁺; ¹H NMR (400 MHz, DMSO-d₆) δ |
| | 11.04 (d, 1H, J = 23.2 Hz), 8.28 (d, 2H, J = 18.8 Hz), 7.56- |
| | 7.53 (m, 1H), 7.32 (dd, 1H, J = 8.8 Hz, 2.8 Hz), 7.09 (dd, |
| | 1H, J = 8.8 Hz, 2.0 Hz), 6.85 (d, 1H, J = 38.4 Hz), |
| | 5.00-4.91 (m, 1H), 4.02-3.70 (m, 8H), 3.28-3.24 (m, 1H), |
| | 2.95-2.67 (m, 2H), 2.51-2.35 (m, 8H), 2.23 (d, 3H, J = |
| | 10.4 Hz), 2.22 (s, 3H) |

### Example 41

### Preparation of Compound 492

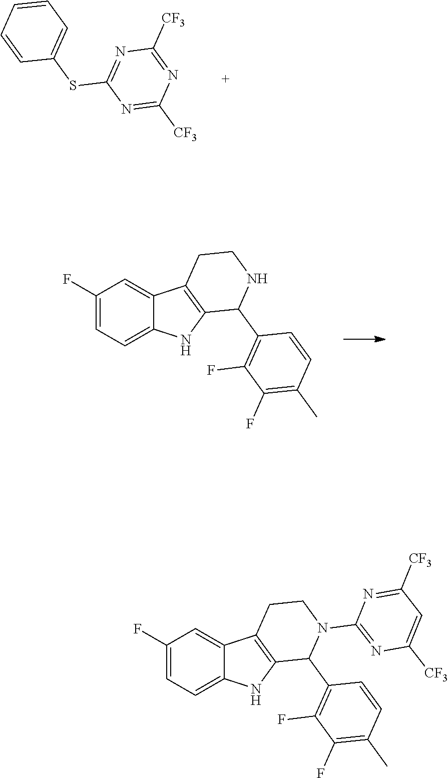 [SMILES: FC(F)(F)c1nc(Sc2ccccc2)nc(C(F)(F)F)n1 ; Cc1ccc(C2NCCc3c2[nH]c2ccc(F)cc32)c(F)c1F ; Cc1ccc(C2c3[nH]c4ccc(F)cc4c3CCN2c2nc(C(F)(F)F)cc(C(F)(F)F)n2)c(F)c1F]

To a 1-neck RBF, equipped with a N₂ inlet, was added: 1-(2,3-difluoro-4-methyl-phenyl)-6-fluoro-2,3,4,9-tetrahydro-1H-pyrido[3,4-b]indole (100 mg, 0.3162 mmol) and 2-phenylsulfanyl-4,6-bis(trifluoromethyl)-1,3,5-triazine (100 mg, 0.3075 mmol), triethylamine (0.1 mL, 0.7 mmol) and THF (5 mL, 61.4 mmol). The reaction mixture was stirred for 4 hours at 50° C. The desired product mass was observed by LCMS, cooled to rt, and concentrated, then the residue was purified by prep-HPLC and silica gel chromatography to afford Compound 492 (20 mg, 0.03764 mmol, 11.90% yield) as a white solid. MS m/z=531.0, [M+H]⁺; (DMSO, 400 MHz), δ 11.08 (s, 1H), 7.32-7.28 (m, 2H), 7.22 (s, 1H), 7.08-7.04 (m, 1H), 6.97-6.92 (m, 1H), 6.87-6.83 (m, 1H), 4.95-4.91 (m, 1H), 3.58-3.52 (m, 1H), 3.02-2.90 (m, 2H), 2.26 (dd, J=1.6, 3H).

### Example 42

### Preparation of Compound 511

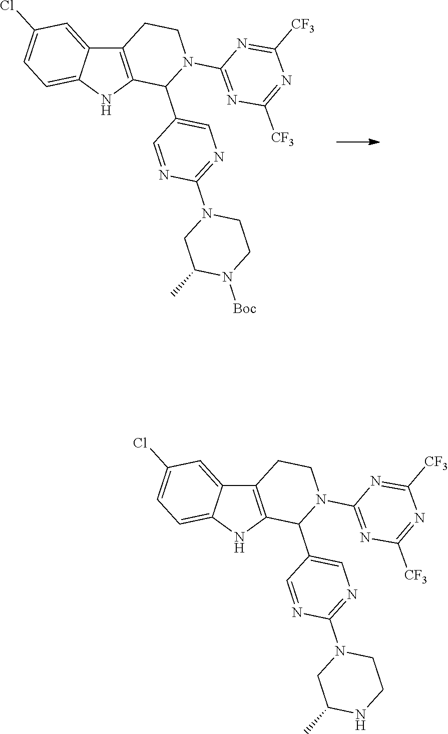 [SMILES: C[C@@H]1CN(c2ncc(C3c4[nH]c5ccc(Cl)cc5c4CCN3c3nc(C(F)(F)F)nc(C(F)(F)F)n3)cn2)CCN1C(=O)OC(C)(C)C ; C[C@@H]1CN(c2ncc(C3c4[nH]c5ccc(Cl)cc5c4CCN3c3nc(C(F)(F)F)nc(C(F)(F)F)n3)cn2)CCN1]

To a 5 mL screw-cap vial, was added: tert-butyl (2R)-4-[5-[(1S)-2-[4,6-bis(trifluoromethyl)pyrimidin-2-yl]-6-chloro-1,3,4,9-tetrahydropyrido[3,4-b]indol-1-yl]pyrimidin-2-yl]-2-methyl-piperazine-1-carboxylate (285 mg, 0.4088 mmol) dissolved in dichloromethane (2 mL, 31.20 mmol). Trifluoroacetic acid anhydride (0.6 mL, 4 mmol) was added at RT and the mixture was stirred for 1 hr. After 1 h the reaction was complete by LCMS. The solvent was removed in vacuo and the residue was purified by silica gel chromatography to give Compound 511 (180 mg, 0.3015 mmol, 73.75% yield) as a white solid. MS m/z=597.2, [M+H]⁺; ¹H NMR (400 MHz, DMSO) δ 11.10 (s, 1H), 8.37 (s, 2H), 7.57 (d, J=2.0 Hz, 1H), 7.55 (s, 1H), 7.33 (d, J=8.8 Hz, 1H), 7.10 (dd, J=8.8 Hz, 1H), 6.78 (s, 1H), 4.98 (dd, J=13.6 Hz, 1H), 4.52-4.56 (m, 2H), 3.48-3.55 (m, 1H), 3.19-3.22 (m, 1H), 2.98-3.12 (m, 3H), 2.80-2.88 (m, 3H), 1.17 (d, J=6.4 Hz, 3H).

Using the procedure described for Compound 511 above, additional compounds described herein were prepared by substituting the appropriate starting materials, suitable reagents, and reaction conditions, obtaining compounds such as those selected from:

| Cpd | Data |
|---|---|
| 517 | MS m/z = 597.1 [M + H]⁺; ¹H NMR (CDCl₃, 400 MHz) δ 9.94 |
| | (s, 1H), 9.30 (s, 1H), 8.41 (s, 2H), 7.91 (s, 1H), 7.53-7.53 (d, J = |
| | 1.6 Hz, 1H), 7.22-7.20 (d, J = 8.8 Hz, 1H), 7.16-7.14 (dd, J = 8.8 |
| | Hz, 1.6 Hz, 1H), 7.11 (s, 1H), 6.91 (s, 1H), 5.22-5.18 (dd, J = |
| | 3.2 Hz, 12.0 Hz, 1H), 4.77-4.74 (d, J = 12.0 Hz, 2H), 3.43-3.33 |
| | (m, 3 H), 3.19-3.13 (m, 2H), 3.02-2.96 (m, 3H), 1.37-1.36 (d, J = |
| | 5.2 Hz 3 H). |

### Example 43

### Preparation of Compound 521

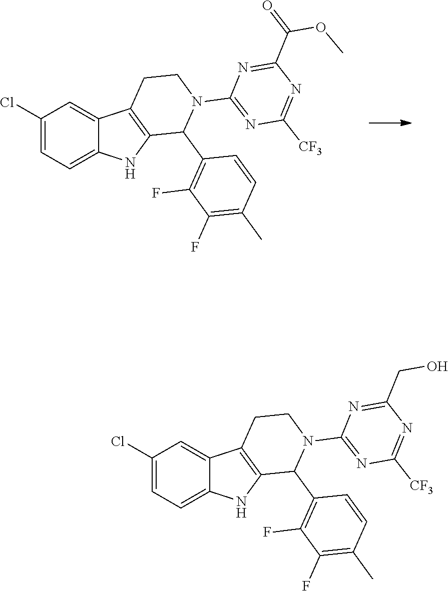 [SMILES: COC(=O)c1nc(N2CCc3c([nH]c4ccc(Cl)cc34)C2c2ccc(C)c(F)c2F)nc(C(F)(F)F)n1 ; Cc1ccc(C2c3[nH]c4ccc(Cl)cc4c3CCN2c2nc(CO)nc(C(F)(F)F)n2)c(F)c1F]

A mixture of methyl 4-[(1S)-6-chloro-1-(2,3-difluoro-4-methyl-phenyl)-1,3,4,9-tetrahydropyrido[3,4-b]indol-2-yl]-6-(trifluoromethyl)-1,3,5-triazine-2-carboxylate (100 mg, 0.1859 mmol) in THF (3 mL, 36.9 mmol) was cooled to −5° C., DIBAL-H (1 mol/L) in hexanes (1.9 mL, 1.9 mmol) added in portions. Stirred the reaction mixture for 2 Hours at −5 C and then allowed to warm to RT. The reaction mixture was filtered, the filtrate was evaporated, and the residue was purified by prep-HPLC to give Compound 521 (40 mg, 0.07845 mmol, 42.20% yield) as yellow solid. MS m/z=509.9, [M+H]⁺; (CDCl3, 400 MHz): 7.96 (s, 1H), 7.51 (s, 1H), 7.29-7.27 (d, J=8.8 Hz, 1H), 7.24-7.21 (dd, J=1.6 Hz, 8.8 Hz, 1H), 7.17-7.14 (dd, J=2.0 Hz, 8.8 Hz, 1H), 6.90-6.76 (m, 2H), 5.27-5.18 (m, 1H), 4.67 (s, 2H), 3.58-3.51 (m, 1H), 3.30-3.24 (m, 1H), 2.99-2.96 (m, 2H), 2.29 (s, 3H).

### Example 44

### Preparation of Compound 526

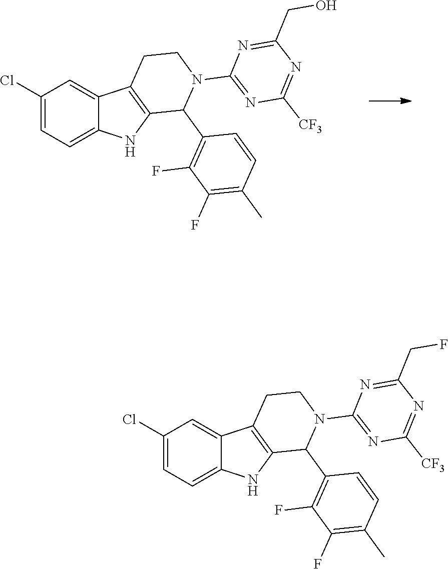 [SMILES: Cc1ccc(C2c3[nH]c4ccc(Cl)cc4c3CCN2c2nc(CO)nc(C(F)(F)F)n2)c(F)c1F ; Cc1ccc(C2c3[nH]c4ccc(Cl)cc4c3CCN2c2nc(CF)nc(C(F)(F)F)n2)c(F)c1F]

To a round bottom flask was added [4-[(1S)-6-chloro-1-(2,3-difluoro-4-methyl-phenyl)-1,3,4,9-tetrahydropyrido[3,4-b]indol-2-yl]-6-(trifluoromethyl)-1,3,5-triazin-2-yl]methanol (1.0 g, 2.0 mmol), DCM (20 mL, 312.0 mmol). The mixture was cooled to 0° C. and diethylaminosulfur trifluoride (500 mg, 3.1019 mmol) was added and the mixture was stirred for 2 Hours at 0° C. The reaction was quenched with saturated NaHCO3, the organic phase was dried over Na2SO4, and evaporated in vacuo. The residue was purified by Prep-HPLC and the resulting product was lyophilized to provide Compound 526 (139 mg, 0.2716 mmol, 14% yield). ESI-MS: m/z=510.0 [M−H]− (DMSO-d₆, 400 MHz), : 11.19 (s, 1H), 7.58-7.56 (d, J=4.8 Hz, 1H), 7.34-7.23 (m, 2H), 7.11-7.09 (dd, J=2.0 Hz, 8.8 Hz, 1H), 7.07-7.04 (t, J=2.4 Hz, 1H), 6.85-6.80 (q, J=2.4 Hz, 1H), 5.58-5.49 (m, 1H), 5.46-5.37 (m, 1H), 5.04-4.89 (dd, J=4.8 Hz, 13.2 Hz, 1H), 3.51-3.42 (m, 1H), 3.02-2.96 (dt, J=4.4 Hz, 15.6 Hz, 1H), 2.91-2.83 (td, J=5.2 Hz, 11.2 Hz, 1H), 2.26 (s, 3H).

### Example 45

### Preparation of Compound 534

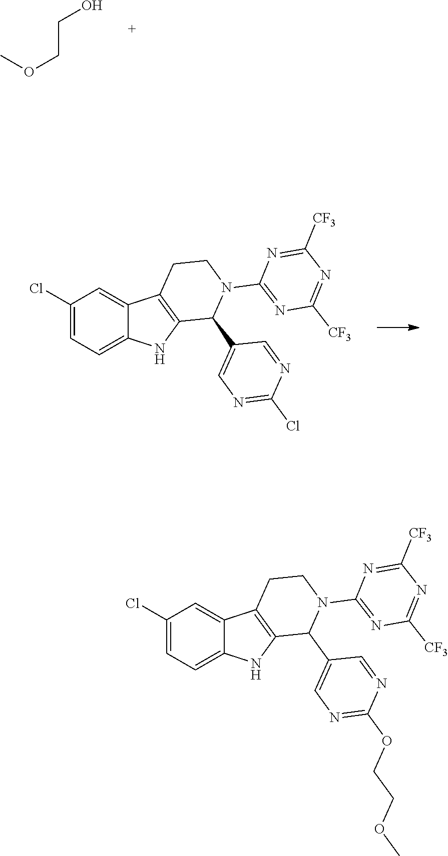

To a 25 mL single neck round-bottomed flask was added a solution of 2-methoxyethanol (13 mg, 0.1709 mmol) in THF (3.0 mL, 37 mmol). The mixture was cooled and maintained at OC. Sodium hydride (2 mg, 0.0791736 mmol) was added into the solution, the mixture was stirred at OC for 15 min. (1S)-2-[4,6-bis(trifluoromethyl)-1,3,5-triazin-2-yl]-6-chloro-1-(2-chloropyrimidin-5-yl)-1,3,4,9-tetra hydropyrido[3,4-b]indole (30 mg, 0.05616 mmol) was added in one portion and the reaction mixture was stirred at room-temperature for two hours. The resulting mixture was monitored by LCMS where the desired product mass was detected. Water was added into the mixture, and extracted with ethyl acetate. The organics were concentrated in vacuo and the crude residue was purified by silica gel chromatography to give Compound 534 (20 mg, 0.03485 mmol, 62.05% yield, 0.6205) as white solid. MS m/z=574.9 [M+H]⁺; ¹H NMR (400 MHz, DMSO) δ 11.12 (s, 1H), 8.56 (s, 2H), 7.60 (d, J=2.4 Hz, 1H), 7.36 (d, J=8.4 Hz, 1H), 7.12 (q, J=8.0 Hz, 1H), 6.96 (s, 1H), 4.98 (q, J=12.0 Hz, 1H), 4.40 (q, J=8.0 Hz, 2H), 3.64 (q, J=4.0 Hz, 2H), 3.55-3.58 (m, 1H), 3.28 (s, 3H), 3.05 (q, J=16.0 Hz, 1H), 2.82-2.91 (m, 1H).

### Example 46

### Preparation of Compound 535

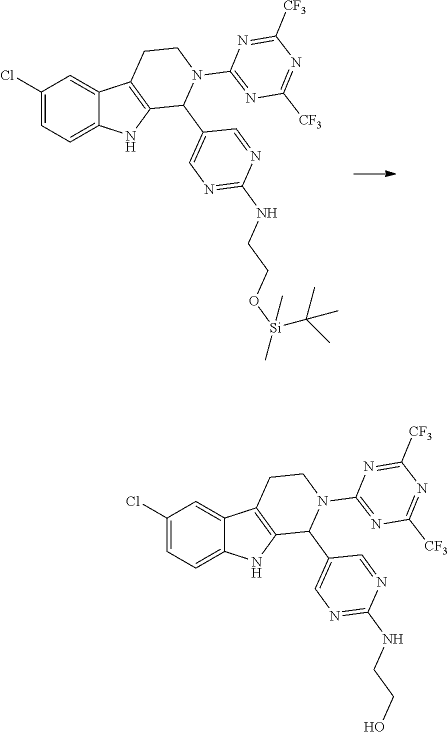 [SMILES: CC(C)(C)[Si](C)(C)OCCNc1ncc(C2c3[nH]c4ccc(Cl)cc4c3CCN2c2nc(C(F)(F)F)nc(C(F)(F)F)n2)cn1 ; OCCNc1ncc(C2c3[nH]c4ccc(Cl)cc4c3CCN2c2nc(C(F)(F)F)nc(C(F)(F)F)n2)cn1]

To a round bottom flask was added 5-[(1S)-2-[4,6-bis(trifluoromethyl)-1,3,5-triazin-2-yl]-6-chloro-1,3,4,9-tetrahydropyrido[3,4-b]indol-1-yl]-N-[2-[tert-butyl (dimethyl)silyl]oxyethyl]pyrimidin-2-amine (250 mg, 0.3714 mmol) and purged with nitrogen. A solution of tetrabutylammonium fluoride (1 mol/L) in THF (0.5 mL, 0.5 mmol) was added and the mixture was stirred at RT for 2 hrs. The reaction mixture was then poured into a separatory funnel with EtOAc and water. The organics were washed with water, brine, and dried over Na2SO4. The organics were concentrated and the residue was purified by prep-HPLC to give Compound 535 (150 mg, 0.2684 mmol, 72.26% yield) as white solid. MS m, =559.1 [M+H]⁺; ¹H NMR (400 MHz, DMSO) δ 11.14 (s, 1H), 8.22 (S, 2H), 7.58 (d, J=2.0 Hz, 1H), 7.34 (d, J=8.4 Hz, 1H), 7.29 (t, J=6.0 Hz, 1H), 7.11 (dd, J=8.8 Hz, 2.0 Hz, 1H), 6.82 (s, 1H), 4.96 (dd, J=13.2 Hz, 5.2 Hz, 1H), 4.63 (t, J=5.6 Hz, 1H), 3.57-3.50 (m, 1H), 3.46 (dd, J=12.0 Hz, 6.0 Hz, 2H), 3.29 (d, 2H), 3.06-3.01 (m, 1H), 2.88-2.81 (m, 1H).

### Example 47

### Preparation of 2-(5-chloro-4-fluoro-1H-indol-3-yl)ethanamine

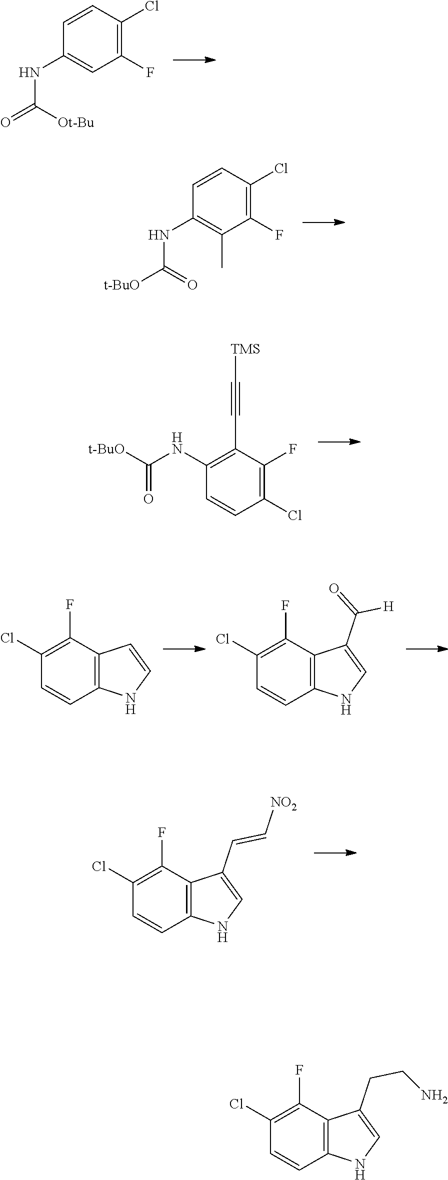

Step 1: A solution of tert-butyl N-(4-chloro-3-fluoro-phenyl)carbamate (79 g, 321.6 mmol) in anhydrous tetrahydrofuran (640 mL) was cooled using a dry ice/acetone plus liquid nitrogen bath, ensuring an internal temperature of −80° C.<T<−75° C. at all times during the reaction. n-Butyllithium (2.5 M in hexane, 405 mL, 1.011 mol) was added over 2H ensuring the drops were thoroughly dispersed but not splashed. Upon complete addition the mixture was stirred for 40 min, a solution of iodine (286 g, 1.127 mol) in anhydrous tetrahydrofuran (430 mL+100 mL rinse) was added over 3 h, maintaining an internal temperature of −80° C.<T<−75° C. The reaction mixture was warmed slowly to −30° C. over 14 h at which point nearly complete conversion of starting material to product was observed. A solution of NH₄Cl (41.6 g in 160 mL water) was added, followed by a solution of NaHSO₃ (184.8 g in 560 mL water) ensuring internal T<−10° C. The quenched mixture was warmed to 10° C. whilst stirring for 1.5 h, then water (500 mL) was added and 1370 mL of THF distillate was removed under reduced pressure at 40° C. The mixture was stirred vigorously at 4° C. for 15 h and filtered. After washing with water (400 mL) a beige solid was obtained with a colorless filtrate which contained no product by HPLC. The solid was digested with hot (65° C.) ethanol (700 mL) for 1H and filtered whilst hot to remove insoluble material (3.5 g) which contained no product by HPLC. The clear, red filtrate obtained was concentrated at 45° C. under vacuum to a volume of 150 mL at which point solids began to precipitate. Water (32 mL) was added to the stirred mixture which was then cooled slowly to 4° C. over 1.5 h. After filtration under vacuum and washing with 4° C. ethanol/water, 2:1 (250 mL) the solid was dried under vacuum at 40° C. for 5 h to give tert-butyl N-(4-chloro-3-fluoro-2-iodo-phenyl)carbamate (C, 103 g, 277.20 mmol, 86.206% yield) as a pale yellow solid.

Step 2: Phosphoryl chloride (22.0 g, 156 mmol) was added to DMF (250 mL) solution at 0° C. under N₂ atmosphere. The mixture was warmed to RT and stirred for 1 Hour, then mixture then cooled back to 0° C., and 5-chloro-4-fluoro-1H-indole (18.4 g, 109 mmol) in 50 mL DMF was added to the mixture drop-wise. The reaction was warmed to RT and stirred for 2 Hours. The reaction was cooled to 0° C. quenched with 20 mL of 15% aq. NaOH solution, followed by 50 mL of water. The mixture was heated to 100° C. for 2 h and cooled to rt, resulting in a slurry. This was filtered and the solids were washed with water and then dried to give the product 5-chloro-6-fluoro-1H-indole-3-carbaldehyde (17.6 g, 89.1 mmol, 82.1% yield) as a yellow solid. MS m/z 198.0 [M+H]⁺.

Step 3: 5-chloro-4-fluoro-1H-indole-3-carbaldehyde (9.5 g, 48 mmol) and ammonium acetate (5.0 g, 65 mmol) were dissolved in 80 mL of nitromethane and stirred at 75° C. for 2 Hours. The mixture was cooled to 0° C. resulting in precipitate formation. The solid was collected by filtration, washed by water, and dried under vacuum to provide the product 5-chloro-4-fluoro-3-[(E)-2-nitrovinyl]-1H-indole (10.3 g, 42.8 mmol, 89% yield) as a yellow solid. MS m/z 241.0 [M+H]⁺.

Step 4: Lithium aluminum hydride (7.6 g, 200 mmol) was placed in a flask under a nitrogen atmosphere and suspended in 50 mL of THF. The mixture was cooled to 0° C. and a solution of 5-chloro-4-fluoro-3-[(E)-2-nitrovinyl]-1H-indole (9.6 g, 40 mmol) in 50 mL of THF was added dropwise. After addition the mixture was heated to 65° C. and stirred for 2 Hr. Upon completion the mixture was cooled down to 0° C. and quenched by addition of 8 mL water followed by addition of 8 mL of 15% aq. NaOH solution. Stirred the mixture at RT for 30 min then MgSO₄ was added and the solids were removed by filtration. The solution was concentrated under reduced pressure providing 2-(5-chloro-6-fluoro-1H-indol-3-yl)ethanamine (8.2 g, 38.5 mmol, 96.4% yield) as a yellow oil. MS m/z 213.4 [M+H]⁺.

### Example 48

### Preparation of Compound 199

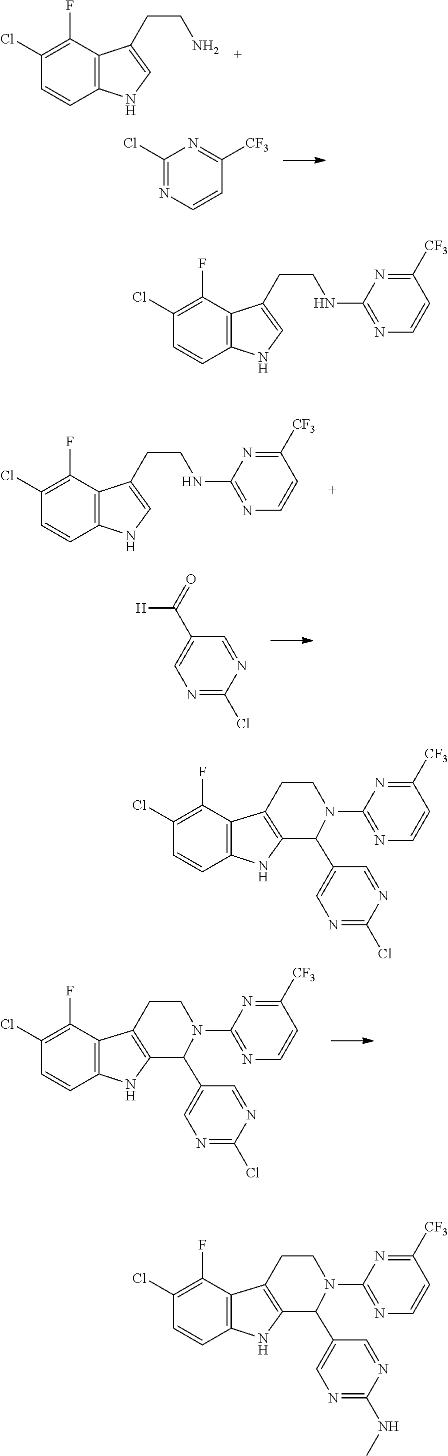

Step 1: A 100 mL round bottomed flask equipped with a reflux condenser was subsequently charged with 2-(5-chloro-4-fluoro-1H-indol-3-yl)ethanamine (1.8 g, 8.5 mmol), 1-butanol (20 mL), triethylamine (3.5 mL, 25 mmol) and 2-chloro-4-(trifluoromethyl)pyrimidine (2.0 g, 11 mmol). The reaction mixture was allowed to stir at reflux for 16 h, after which volatiles were evaporated under reduced pressure and the resulting dark oily residue subjected to silica-gel chromatography (petroleum ether:EtOAc gradient 0 to 40%). After purification N-[2-(5-chloro-4-fluoro-1H-indol-3-yl)ethyl]-4-(trifluoromethyl)pyrimidin-2-amine (2.5 g, 7.0 mmol, 82% yield) was isolated as an off-white amorphous solid.

Step 2. An 8 mL vial equipped with a stir bar was charged with N-[2-(5-chloro-4-fluoro-1H-indol-3-yl)ethyl]-4-(trifluoromethyl)pyrimidin-2-amine (100 mg, 0.279 mmol) and 2-butanol (3.0 mL), then 2-chloropyrimidine-5-carbaldehyde (50 mg, 0.351 mmol) was added by micro syringe followed by toxic acid (30 mg, 0.174 mmol). The vial was allowed to stir at 95° C. for 10 h. After being allowed to cool to ambient temperature, volatiles were removed under reduced pressure and the resulting dark residue taken up in ethylacetate (1 mL) leading to the formation of a suspension. The solid was filtered of yielding crude 6-chloro-1-(2-chloropyrimidin-5-yl)-5-fluoro-2-[4-(trifluoromethyl)pyrimidin-2-yl]-1,3,4,9-tetrahydropyrido[3,4-b]indole (350 mg, 25% purity, 65% yield).

Step 3: An 8 mL vial equipped with a stir bar was charged with 6-chloro-1-(2-chloropyrimidin-5-yl)-5-fluoro-2-[4-(trifluoromethyl)pyrimidin-2-yl]-1,3,4,9-tetrahydropyrido [3,4-b]indole (350 mg, 0.181 mmol, 25% purity), tetrahydrofuran (5.0 mL), methylamine hydrochloride (63 mg, 0.93 mmol) and triethylamine (815 mg, 8.0 mmol). The reaction mixture was stirred over night at reflux. After being allowed to cool to ambient temperature volatiles were removed under reduced pressure and the residue purified by silica gel chromatography. Compound 199 (63 mg, 0.132 mmol, 73% yield) was isolated as an off-white solid. MS m/z=476.0 [M−H]⁻; ¹H NMR (400 MHz, DMSO) δ 11.40 (s, 1H), 8.79 (d, J=4.8 Hz, 1H), 8.25 (s, 2H), 7.27-7.23 (m, 1H), 7.18-7.12 (m, 3H), 6.78 (s, 1H), 5.07-4.94 (m, 1H), 3.45-3.38 (m, 1H), 3.09-2.93 (m, 2H), 2.75 (d, J=4.8 Hz, 3H).

Using the procedure described for Compound 199 above, additional compounds described herein may be prepared by substituting the appropriate starting materials, suitable reagents, and reaction conditions, obtaining compounds such as those selected below (*Compound isolated as a single enantiomer by chromatographic separation using preparative chiral SFC):

| Cpd | Data |
|---|---|
| 104 | MS m/z = 462.0 [M + H]⁺; ¹H NMR (DMSO-d, 400 MHz) δ 11.57 (s, 1H), 8.72 (d, |
| | J = 4.4 Hz, 1H), 7.84 (d, J = 8.0 Hz, 2H), 7.56 (d, J = 6.4 Hz, 2H), 7.23-7.13 |
| | (m, 4H), 4.96-4.93 (m, 1H), 3.91(s, 3H), 3.28-3.20 (m, 1H), 3.10-2.96 (m, 2H). |
| 109 | MS m/z = 425.1 [M + H]⁺; ¹H NMR (DMSO-d, 400 MHz) δ 11.49 (s, 1H), 8.36 (d, |
| | J = 1.6 Hz, 1H), 7.20-7.09 (m, 6H), 7.03 (s, 1H), 4.79-4.75 (m, 1H), 3.22-3.15 (m, |
| | 1H), 2.97-2.90 (m, 2H), 2.37 (d, J = 2.4 Hz, 3H), 2.27 (s, 3H). |
| 110 | MS m/z = 431.9 [M + H]⁺; ¹H NMR (DMSO-d, 400 MHz) δ, 11.49 (s, 1H), 7.20- |
| | 6.91 (m, 8H), 4.95-4.67 (m, 1H), 3.27-3.18 (m, 1H), 3.02-2.92 (m, 2H), 2.43-2.37 |
| | (m, 3H), 2.27 (s, 3H). |
| 111 | MS m/z = 436.1 [M + H]⁺; ¹H NMR (DMSO-d, 400 MHz) δ 11.50 (s, 1H), 7.36-7.33 |
| | (m, 2H), 7.22-6.94 (m, 6H), 4.98-4.68 (m, 1H), 3.25-3.18 (m, 1H), 3.03-2.94 (m, |
| | 2H), 2.47-2.34 (m, 3H). |
| 113 | MS m/z = 447.1 [M + H]⁺; ¹H NMR (DMSO-d, 400 MHz) δ 11.43 (s, 1H), 8.39 (d, |
| | J = 1.6 Hz, 1H), 7.45-7.38 (m, 1H), 7.31 (s, 1H), 7.18-7.11 (m, 3H), 6.89-6.85 (m, |
| | 1H), 5.00-4.76 (m, 1H), 3.34-3.28 (m, 1H), 2.99-2.35 (m, 2H), 2.36 (d, J = 2.4 Hz, |
| | 3H). |
| 114 | MS m/z = 461.0 [M + H]⁺; ¹H NMR (DMSO-d, 400 MHz) δ 11.41 (s, 1H), 8.37 (d, |
| | J = 1.6 Hz, 1H), 7.26 (s, 1H), 7.17-7.10 (m, 2H), 7.04-7.00 (m, 1H), 6.76-6.72 (m, |
| | 1H), 4.79-4.75 (m, 1H), 3.35-3.27(m, 1H), 2.98-2.94 (m, 2H), 2.35 (d, J = 2.4 Hz, |
| | 3H), 2.26 (d, J = 1.2 Hz, 3H). |
| 116 | MS m/z = 439.1 [M + H]⁺; ¹H NMR (DMSO-d, 400 MHz) δ 11.46 (s, 1H), 7.19- |
| | 7.09 (m, 6H), 7.04 (s, 1H), 4.80-4.76 (m, 1H), 3.19-3.11 (m, 1H), 2.99-2.89 (m, |
| | 2H), 2.32 (d, J = 2.4 Hz, 6H), 2.26 (s, 3H). |
| 117 | MS m/z = 461.1 [M + H]⁺; ¹H NMR (DMSO-d, 400 MHz) δ 11.41 (s, 1H), 7.43- |
| | 7.36 (m, 1H), 7.28 (s, 1H), 7.18-7.08 (m, 3H), 6.89-6.86 (m, 1H), 4.80-4.77 |
| | (m, 1H), 3.31-3.25 (m, 1H), 3.01-2.90 (m, 2H), 2.31 (d, J = 2.0 Hz, 6H). |
| 118 | MS m/z = 474.9 [M + H]⁺; ¹H NMR (DMSO-d, 400 MHz) δ 11.38 (s, 1H), 7.32 |
| | (s, 1H), 7.15-7.09 (m, 2H), 7.00 (t, J = 7.4 Hz, 1H), 6.75 (t, J = 7.2 Hz, 1H), 4.80- |
| | 4.76 (m, 1H), 3.32-3.26 (m, 1H), 2.98-2.91 (m, 2H), 2.31 (d, J = 2.8 Hz, 6H), 2.25 |
| | (d, J = 1.2 Hz, 3H). |
| 135 | MS m/z = 407.0 [M + H]⁺; ¹H NMR (DMSO-d, 400 MHz) δ 11.49 (s, 1H), 8.30 (s, |
| | 2H), 7.19-7.11 (m, 6H), 7.09-7.08 (m, 1H), 4.82-4.78 (m, 1H), 3.19-3.12 (m, 1H), |
| | 2.98-2.89 (m, 2H), 2.27 (s, 3H), 2.10 (s, 3H). |
| 137 | MS m/z = 443.1 [M + H]⁺; ¹H NMR (DMSO-d, 400 MHz) δ 11.40 (s, 1H), 8.31 (s, |
| | 2H), 7.35 (s, 1H), 7.17-7.10 (m, 2H), 7.04-7.00 (m, 1H), 6.73-6.70 (m, 1H), 4.80- |
| | 4.76 (m, 1H), 3.29-3.23 (m, 1H), 2.95-2.92 (m, 2H), 2.26 (s, 3H), 2.10 (s, 3H). |
| 146 | MS m/z = 464.2 [M − H]⁻; ¹H NMR (DMSO-d, 400 MHz) δ 11.45 (s, 1H), 8.81 (d, |
| | J = 4.8 Hz, 1H), 8.56 (d, J = 2.8 Hz, 1H), 8.54-8.49 (m, 1H), 7.65-7.61 (m, 1H), 7.21- |
| | 7.14 (m, 3H), 7.10-6.97 (m, 1H), 5.04-4.88 (m, 1H), 3.49-3.39 (m, 1H), 3.10-2.94 |
| | (m, 2H) |
| 147 | MS m/z = 497.0 [M + H]⁺; ¹H NMR (DMSO-d, 400 MHz) δ 11.25 (s, 1H), 7.69 (d, |
| | J = 7.2 Hz, 1H), 7.44-7.38 (m, 1H), 7.35-7.29 (m, 2H), 7.16-7.11 (m, 1H), 7.07 (s, |
| | 1H), 6.90 (t, J = 7.0 Hz, 1H), 4.92 (br, 1H), 3.42-3.39 (m, 1H), 2.93-2.78 (m, 2H), |
| | 2.46 (s, 3H). |
| 149 | MS m/z = 523.0 [M − H]⁻; ¹H NMR (DMSO-d, 400 MHz) δ 11.54 (s, 1H), 8.82 (d, |
| | J = 4.4 Hz, 1H), 7.95-7.92 (m, 2H), 7.63-7.61 (m, 2H), 7.21-7.14 (m, 4H), 5.00- |
| | 4.85 (m, 1H), 3.31-3.29 (m, 1H), 3.20 (s, 3H), 3.05-3.01 (m, 2H). |
| 150 | MS m/z = 524.1 [M − H]⁻; ¹H NMR (DMSO-d, 400 MHz) δ 11.53 (s, 1H), 8.81 (d, |
| | J = 4.4 Hz, 1H), 7.82-7.80 (m, 2H), 7.55-7.53 (m, 2H), 7.36 (s, 2H), 7.20-7.14 (m, |
| | 4H), 5.02-4.82 (m, 1H), 3.32-3.29 (m, 1H), 3.07-3.01 (m, 2H). |
| 160 | MS m/z = 507.0 [M − H]⁻; ¹H NMR (DMSO-d, 400 MHz) δ 11.42 (s, 1H), 8.79 (d, |
| | J = 4.8 Hz, 1H), 7.98-7.94 (m, 1H), 7.34 (dd, J = 14.4, 2.0 Hz, 1H), 7.18-7.11 (m, |
| | 3H), 6.97-6.85 (m, 1H), 5.02-4.88 (m, 1H), 3.45-3.39 (m, 1H), 3.11-2.90 (m, 8H). |
| 177 | MS m/z = 488.9 [M − H]⁻; ¹H NMR (DMSO-d, 400 MHz) δ 11.43 (s, 1H), 8.78 (d, |
| | J = 3.6 Hz, 1H), 8.09 (d, J = 2.0 Hz, 1H), 7.41-7.38 (m, 1H), 7.16-7.13 (m, 2H), |
| | 7.11-7.10 (m, 1H), 6.98-6.86 (m, 1H), 6.59 (d, J = 8.8 Hz, 1H), 5.01-4.88 (m, 1H), |
| | 3.32-3.30 (m, 1H), 3.07-2.99 (m, 2H), 2.97 (s, 6H). |
| 178 | MS m/z = 492.1 [M + H]⁺; ¹H NMR (DMSO-d, 400 MHz) δ 11.38 (s, 1H), 8.79 (d, |
| | J = 4.8 Hz, 1H), 8.31 (s, 2H), 7.18-7.12 (m, 3H), 6.79 (s, 1H), 5.05-4.94 (m, 1H), |
| | 3.46-3.36 (m, 1H), 3.08 (s, 6H), 3.06-2.93 (m, 2H). |
| 181* | MS m/z = 479.1 [M − H]⁻; ¹H NMR (DMSO-d, 400 MHz) δ 11.53 (br s, 1H), 8.80 |
| | (d, J = 4.8 Hz, 1H), 7.44 (d, J = 8.4 Hz, 2H), 7.34 (d, J = 8.8 Hz, 2H), 7.19-7.01 |
| | (m, 4H), 4.98-4.80 (m, 1H), 3.30-3.24 (m, 1H), 3.05-2.96 (m, 2H). |
| 182* | MS m/z = 479.1 [M − H]⁻; ¹H NMR (DMSO-d, 400 MHz) δ 11.53 (s, broad, 1H), |
| | 8.80 (d, J = 4.8 Hz, 1H), 7.44 (d, J = 8.8 Hz, 2H), 7.34 (d, J = 8.4 Hz, 2H), 7.19- |
| | 7.03 (m, 4H), 4.97-4.84 (m, 1H), 3.30-3.24 (m, 1H), 3.07-2.94 (m, 2H). |
| 186 | MS m/z = 482.0 [M − H]⁻; ¹H NMR (DMSO-d, 400 MHz) δ 11.42 (s, 01H), 8.81 (d, |
| | J = 4.8 Hz, 1H), 7.89 (q, J = 8.0 Hz, 1H), 7.20-7.16 (m, 4H), 7.12 (dd, J = 8.0, 4.0 |
| | Hz, 1H), 4, 94-4, 90 (m, 1H), 3.47-3.35 (m, 1H), 3.08-2.96 (m, 2H). |
| 189* | MS m/z = 475.1 [M + H]⁺; ¹H NMR (DMSO-d, 400 MHz) δ 11.39 (s, 1H), 7.23 (s, |
| | 1H), 7.15-7.09 (m, 2H), 7.01 (t, J = 7.6 Hz, 1H), 6.75 (t, J = 7.2 Hz, 1H), 4.79-4.76 |
| | (m, 1H), 3.31-3.26 (m, 1H), 2.99-2.87 (m, 2H), 2.30 (d, J = 2.0 Hz, 6H), 2.25 (s, |
| | 3H). |
| 190* | MS m/z = 475.1 [M + H]⁺; ¹H NMR (DMSO-d, 400 MHz) δ 11.39 (s, 1H), 7.22 (s, |
| | 1H), 7.14-7.08 (m, 2H), 7.01 (t, J = 7.5 Hz, 1H), 6.75 (t, J = 7.2 Hz, 1H), 4.79-4.76 |
| | (m, 1H), 3.31-3.26 (m, 1H), 2.98-2.85 (m, 2H), 2.31 (d, J = 2.1 Hz, 6H), 2.27 (s, |
| | 3H). |
| 191 | MS m/z = 449.2 [M + H]⁺; ¹H NMR (DMSO-d, 400 MHz) δ 11.43 (br, 1H), 9.15 (s, |
| | 1H), 8.83-8.81 (m, 3H), 7.25-7.14 (m, 3H), 6.98 (d, J = 3.0 Hz, 1H), 5.00 (d, J = |
| | 13.2 Hz, 1H), 3.52-3.46 (m, 1H), 3.09-3.02 (m, 2H). |
| 193 | MS m/z = 513.0 [M − H]⁻; ¹H NMR (DMSO-d, 400 MHz) δ 11.40 (s, 1H), 8.87 (s, |
| | 2H), 8.82 (d, J = 4.8 Hz, 1H), 8.55 (s, 1H), 7.92 (s, 1H) 7.21-7.14 (m, 4H), 6.97 |
| | (br, 1H), 5.05-5.02 (m, 1H), 3.58-3.52 (m, 1H), 3.14-3.00 (m, 2H). |
| 194 | MS m/z = 441.1 [M + H]⁺; ¹H NMR (DMSO-d, 400 MHz) δ 11.42 (s, 1H), 8.54 (s, |
| | 2H), 8.05 (d, J = 2.0 Hz, 1H), 7.39-7.37 (m, 1H), 7.16-7.10 (m, 2H), 6.88 (s, 1H), |
| | 6.58 (d, J = 8.8 Hz, 1H), 4.84-4.80 (m, 1H), 3.31-3.23 (m, 1H), 2.97 (s, 8H). |
| 195 | MS m/z = 461.1 [M − H]⁻; ¹H NMR (DMSO-d, 400 MHz) δ 11.43 (s, 1H), 8.54 (s, |
| | 2H), 7.29 (s, 1H), 7.18-7.12 (m, 2H), 7.06-7.02 (m, 1H), 6.76-6.72 (m, 1H), 4.81- |
| | 4.77 (m, 1H), 3.32-3.28(m, 1H), 2.98-2.96 (m, 2H), 2.26 (s, 3H). |
| 196 | MS m/z = 441.0 [M − H]⁻; ¹H NMR (DMSO-d, 400 MHz) δ 11.40 (s, 1H), 8.29 (d, |
| | J = 5.2 Hz, 1H), 7.38 (s, 1H), 7.16-7.10 (m, 2H), 7.04-7.00 (m, 1H), 6.78-6.74 (m, |
| | 1H), 6.61 (d, J = 4.8 Hz, 1H), 4.99-4.86 (m, 1H), 3.31-3.25 (m, 1H), 2.96-2.93 (m, |
| | 2H), 2.32 (s, 3H), 2.26 (d, J = 1.2 Hz, 3H). |
| 197 | MS m/z = 494.9 [M − H]⁻; ¹H NMR (DMSO-d, 400 MHz) δ 11.45 (s, 1H), 8.79 (d, |
| | J = 4.8 Hz, 1H), 7.28 (s, 1H), 7.18-7.12 (m, 3H), 7.06-7.02 (m, 1H), 6.83-6.79 (m, |
| | 1H), 4.91-4.89 (m, 1H), 3.46-3.38 (m, 1H), 3.05-2.95 (m, 2H), 2.26 (d, J = 0.8 Hz, |
| | 3H). |
| 228 | MS m/z = 441.0 [M − H]⁻; ¹H NMR (DMSO-d, 400 MHz) δ 11.52 (s, 1H), 7.87 (s, |
| | 1H), 7.22-7.12 (m, 6H), 6.94 (s, 1H), 4.77-4.73 (m, 1H), 3.35-3.29 (m, 1H), |
| | 3.05-2.97 (m, 2H), 2.28 (s, 3H). |
| 229 | MS m/z = 477.0 [M − H]⁻; ¹H NMR (DMSO-d, 400 MHz) δ 11.44 (s, 1H), 7.89 (s, |
| | 1H), 7.18 (t, J = 8.0 Hz, 2H), 7.13 (s, 1H), 7.06 (t, J = 8.0 Hz, 1H), 6.82 (t, J = 8.0 |
| | Hz, 1H), 4.80-4.76 (m, 1H), 3.38-3.36 (m, 1H), 3.06-3.02 (m, 2H), 2.27 (s, |
| | 3H). |
| 230 | MS m/z = 514.0 [M − H]⁻; ¹H NMR (DMSO-d, 400 MHz) δ 11.49 (s, 1H), 9.43 (s, |
| | 1H), 8.97 (s, 2H), 8.83 (d, J = 4.0 Hz, 1H), 8.33 (s, 1H), 7.22-7.15 (m, 3H), 7.00 |
| | (s, 1H), 5.05 (d, J = 8.0 Hz, 1H), 3.58 (t, J = 12.0 Hz, 1H), 3.06-7.04 (m, 2H). |
| 232 | MS m/z = 468.0 [M + H]⁺; ¹H NMR (DMSO-d, 400 MHz) 6 11.42 (s, 1H), 7.38- |
| | 7.21 (m, 1H), 7.20 (s, 1H), 7.17-7.11 (m, 2H), 7.03 (t, J = 7.4 Hz, 1H), 6.79 (t, J = |
| | 7.2 Hz, 1H), 4.92-4.76 (m, 1H), 3.36-3.36 (m, 1H), 3.03-2.97 (m, 2H), 2.41 (s, |
| | 3H), 2.26 (s, 3H). |

### Example 49

### Preparation of Compound 220

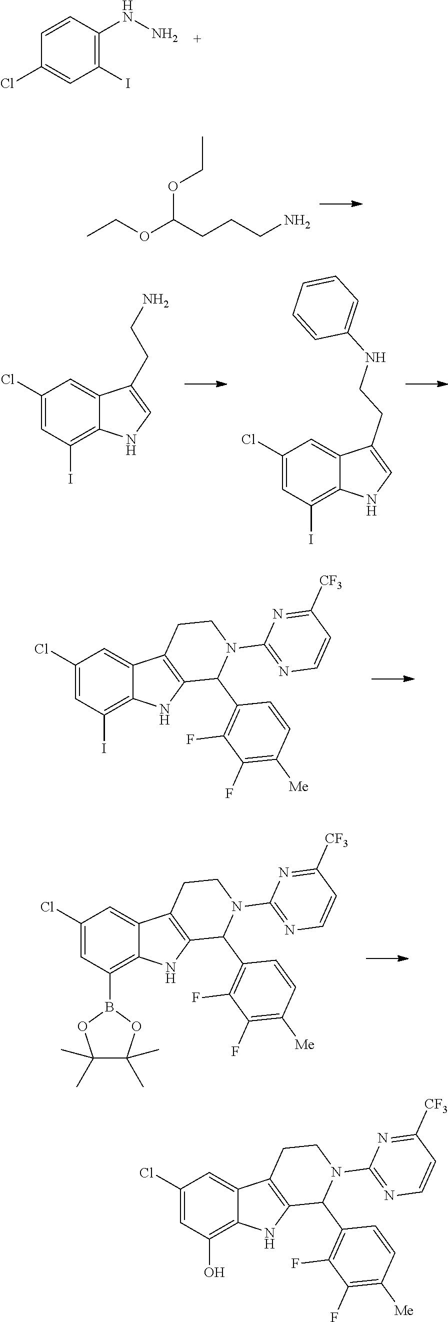

Step 1: A 1 L round-bottom flask was charged with 4,4-diethoxybutan-1-amine (12.00 g, 74.42 mmol) in water (200 mL) and (4-chloro-2-iodo-phenyl)hydrazine (20.0 g, 74.49 mmol). Sulfuric acid (20 mL, 25.6 mmol, 1.28 mol/L) was added and the reaction mixture allowed to stir at 100° C. for 8 h. After the reaction mixture was allowed to cool to 25° C. the formed solid was filtered, the pH adjusted to 7 by addition of NaHCO₃. The resulting solid was filtered off and purified by flash chromatography (eluent MeOH/DCM) to give 2-(5-chloro-7-iodo-1H-indol-3-yl)ethanamine (10 g, 31.2 mmol, 42% yield) as an off-white solid.

Step 2: A 100 mL round-bottom flask equipped with a magnetic stir bar was charged with 2-(5-chloro-7-iodo-1H-indol-3-yl)ethanamine (2.00 g, 6.24 mmol) and suspended in 1-butanol (20 mL). 2-chloro-4-(trifluoromethyl)pyrimidine (1.50 g, 8.22 mmol) and triethylamine (3.0 m L, 22 mmol) were added, the flask equipped with a reflux condenser and the reaction mixture allowed to stir at reflux for 16 h. After being allowed to cool to ambient temperature, volatiles were removed under reduced pressure and the residue purified by column chromatography (eluent petrol ether/ethyl acetate). N-[2-(5-chloro-7-iodo-1H-indol-3-yl)ethyl]-4-(trifluoromethyl)pyrimidin-2-amine (2.2 g, 4.7 mmol, 76% yield) was isolated as an off-white solid.

Step 3: A 50 mL round-bottom flask equipped with a magnetic stir bar was charged with N-[2-(5-chloro-7-iodo-1H-indol-3-yl)ethyl]-4-(trifluoromethyl)pyrimidin-2-amine (0.5 g, 1 mmol), 2-butanol (10 m L, 109 mmol), 2,3-difluoro-4-methyl-benzaldehyde (0.4 g, 3 mmol) and p-toluenesulfonic acid (0.15 g, 0.87 mmol). The flask was equipped with a reflux condenser then the mixture allowed to stir at 100° C. for 16 h. After being allowed to cool to ambient temperature the reaction mixture was diluted with sat. NaHCO₃ (10 mL) and EtOAc (20 mL). The aqueous phase was extracted with EtOAc (3×20 mL), the combined organic phases were concentrated under reduced pressure and the residue subjected to purification by preparatory HPLC. After lyophilization 6-chloro-1-(2,3-difluoro-4-methyl-phenyl)-8-iodo-2-[4-(trifluoromethyl)pyrimidin-2-yl]-1,3,4,9-tetrahydropyrido[3,4-b]indole (0.45 g, 0.74 mmol, 70% yield) was isolated as an amorphous off-white solid.

Step 4: A 25 mL 3-neck round-bottom flask was charged with 6-chloro-1-(2,3-difluoro-4-methyl-phenyl)-8-iodo-2-[4-(trifluoromethyl)pyrimidin-2-yl]-1,3,4,9-tetrahydropyrido [3,4-b]indole (150 mg, 0.248 mmol) in 1,4-dioxane (2.0 mL), followed by 4,4,5,5-tetramethyl-2-(4,4,5,5-tetramethyl-1,3,2-dioxaborolan-2-yl)-1,3,2-dioxaborolane (95 mg, 0.374 mmol,), potassium acetate (50 mg, 0.509 mmol), [1,1′-bis(diphenylphosphino)ferrocene]dichloropalladium(II) (20 mg, 0.026 mmol). The reaction was placed under a nitrogen atmosphere and allowed to stir at 100° C. for 16 h. After being allowed to cool to ambient temperature the reaction mixture was filtered and volatiles removed under reduced pressure. The resulting residue was subjected to prep-TLC purification affording 6-chloro-1-(2,3-difluoro-4-methyl-phenyl)-8-(4,4,5,5-tetramethyl-1,3,2-dioxaborolan-2-yl)-2-[4-(trifluoromethyl)pyrimidin-2-yl]-1,3,4,9-tetrahydropyrido[3,4-b]indole (150 mg, 0.149 mmol, 60% yield) as an amorphous brownish solid.

Step 5: A 20 mL vial equipped with a magnetic stir bar was charged with a solution of 6-chloro-1-(2,3-difluoro-4-methyl-phenyl)-8-(4,4,5,5-tetramethyl-1,3,2-dioxaborolan-2-yl)-2-[4-(trifluoromethyl)pyrimidin-2-yl]-1,3,4,9-tetrahydropyrido[3,4-b]indole (150 mg, 0.1488 mmol) in tetrahydrofuran (1.5 mL) followed by hydrogen peroxide in water (0.3 mL) and sodium hydroxide (0.2 mL, 0.2 mmol, 1 mol/L). The mixture was stirred at ambient temperature for 10 min, then the mixture was concentrated and the residue subjected to prep-HPLC purification to give Compound 220 (40 mg, 0.081 mmol, 54.3% yield) as a white powder. MS m/z=493.0 [M+H]⁺; ¹H NMR (400 MHz, DMSO-d₆) δ 11.03 (s, 1H), 10.05 (s, 1H), 8.79 (d, J=4.4 Hz, 1H), 7.36 (s, 1H), 7.13 (d, J=4.8 Hz, 1H), 7.04-7.00 (m, 1H), 6.98 (d, J=5.6 Hz, 1H), 6.68-6.64 (m, 1H), 6.52 (d, J=1.6 Hz, 1H), 4.92-4.73 (m, 1H), 3.32-3.25 (m, 1H), 2.80-2.79 (m, 2H), 2.26 (s, 3H).

Using the procedure described for Compound 220 above, additional compounds described herein were prepared by substituting the appropriate starting materials, suitable reagents, and reaction conditions, obtaining compounds such as those selected from:

| Cpd | Data |
|---|---|
| 207 | MS m/z = 459.0 [M + H]⁺; ¹H NMR (DMSO-d₆, 400 MHz) δ 11.03 (s, 1H), 9.98 (s, |
| | 1H), 8.77 (s, 1H), 7.18-7.13 (m, 4H), 7.09-7.08 (m, 2H), 6.97-6.96 (m, 1H), 6.51 |
| | (d, J = 2.0 Hz, 1H), 4.95-4.69 (m, 1H), 3.25-3.18 (m, 1H), 2.80-2.77 (m, 2H), |
| | 2.27 (s, 3H). |
| 250 | MS m/z = 477.0 [M − H]⁻; ¹H NMR (400 MHz, DMSO) δ 10.77 (s, 1H), 9.62 (s, 1H), |
| | 8.78-8.75(m, 1H), 7.44-7.38(m, 3H), 7.33 (d, J = 8.0 Hz, 2H), 7.11 (d, J = 8.0 Hz, |
| | 1H), 6.98 (s, 1H), 6.89 (s, 1H), 4.87 (s, 1H), 3.24-3.16(m, 1H), 2.83-2.73(m, 2H). |
| 333 | MS m/z = 489.1 [M + H]⁺. |
| 339 | MS m/z = 492.0 [M + H]⁺; ¹H NMR (400 MHz, DMSO) δ 10.99 (s, 1H), 10.03 (s, |
| | 1H), 8.05 (d, J = 2.4 Hz, 1H), 7.38 (dd, J = 9.0, 2.8 Hz, 1H), 7.03 (s, 1H), 6.97 (d, |
| | J = 1.6 Hz, 1H), 6.78 (d, J = 9.2 Hz, 1H), 6.51 (d, J = 2.0 Hz, 1H), 4.96-4.91 (m, 1H), |
| | 3.67-3.65 (m, 4H), 3.40-3.38 (m, 4H), 3.18-3.16(s, 1H), 2.84-2.68 (m, 2H), |
| | 2.40 (s, 3H), 2.32 (s, 3H). |
| 348 | MS m/z = 455.9 [M + H]⁺; ¹H NMR (400 MHz, DMSO) δ 11.02 (s, 1H), 9.99 (br, |
| | 1H), 7.38 (s, 1H), 7.01 (t, J = 7.2 Hz, 1H), 6.96 (d, J = 1.6 Hz, 1H), 6.66 (t, J = 7.4 |
| | Hz, 1H), 6.51 (d, J = 1.6 Hz, 1H), 4.90-4.86 (m, 1H), 3.22-3.15 (m, 1H), 2.77- |
| | 2.74 (m, 2H), 2.34 (d, J = 8.0 Hz, 6H), 2.25 (s, 3H). |

### Example 50

### Preparation of Compound 325

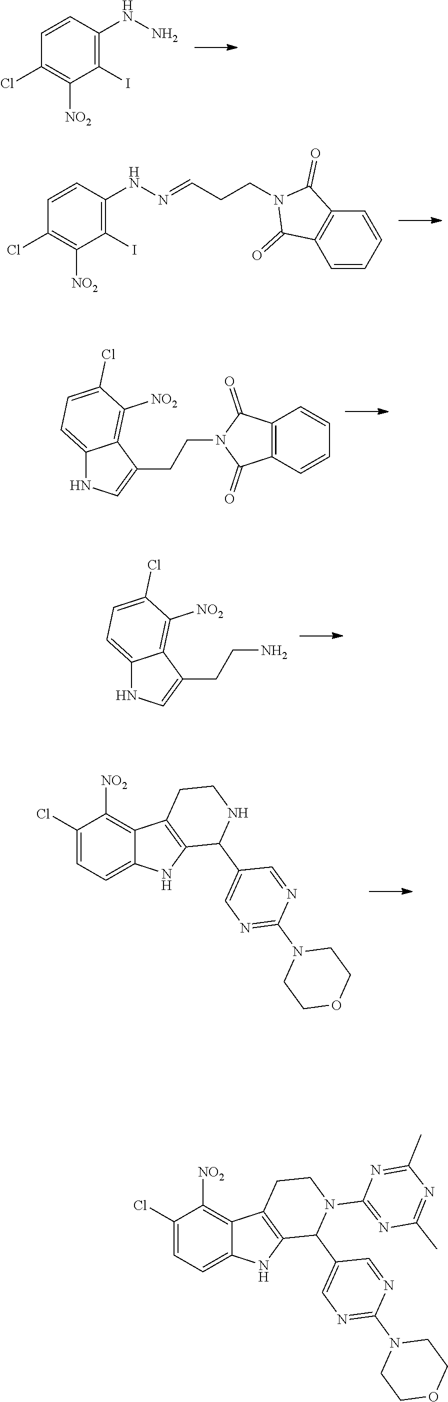 [SMILES: NNc1ccc(Cl)c([N+](=O)[O-])c1I ; O=C1c2ccccc2C(=O)N1CC/C=N/Nc1ccc(Cl)c([N+](=O)[O-])c1I ; O=C1c2ccccc2C(=O)N1CCc1c[nH]c2ccc(Cl)c([N+](=O)[O-])c12 ; NCCc1c[nH]c2ccc(Cl)c([N+](=O)[O-])c12 ; O=[N+]([O-])c1c(Cl)ccc2[nH]c3c(c12)CCNC3c1cnc(N2CCOCC2)nc1 ; Cc1nc(C)nc(N2CCc3c([nH]c4ccc(Cl)c([N+](=O)[O-])c34)C2c2cnc(N3CCOCC3)nc2)n1]

Step 1: A 500 mL single neck round-bottom flask was charged with a solution of (4-chloro-3-nitro-phenyl)hydrazine (20.0 g, 107 mmol) in ethanol (200 mL, 3430 mmol) followed by 2-(4,4-diethoxybutyl)isoindoline-1,3-dione (31.0 g, 106 mmol) and sulfuric acid (14.0 mL, 56.0 mmol, 4 mol/L). The mixture was allowed to stir at 50 C for 3 hours, after which the mixture was filtered and the obtained solid subjected to silica gel purification (petroleum ether:ethyl acetate=3:1) affording 2-[(4Z)-4-[(4-chloro-3-nitro-phenyl)hydrazono]butyl]isoindoline-1,3-dione (24.0 g, 62.0 mmol, 58.2% yield) as yellow solid.

Step 2: A 250 mL single neck round-bottomed flask was charged with polyphosphoric acid (6.0 g) and acetonitrile (50.0 mL, 954 mmol) was allowed to stir at 60 C for 10 minutes, then 2-[(4Z)-4-[(4-chloro-3-nitro-phenyl)hydrazono]butyl]isoindoline-1,3-dione (3.0 g, 7.8 mmol) was added. The mixture was allowed to stir at 90° C. for 30 minutes. After being allowed to cool to ambient temperature the reaction mixture was concentrated under reduced pressure to give a dark residue. The residue was treated with sat. aq. Na₂CO₃ till the pH reached 8, and extracted with ethyl acetate (3*50.0 mL). The organic phases were combined and concentrated to give a dark residue which was subjected to purification by silica gel chromatography (eluent dichloromethane:methanol=10:1). 2-[2-(5-chloro-4-nitro-1H-indol-3-yl)ethyl]isoindoline-1,3-dione (1.5 g, 4.1 mmol, 52% yield) was isolated as yellow solid.

Step 3: A 100 mL single neck round-bottomed flask was charged with a solution of 2-[2-(5-chloro-4-nitro-1H-indol-3-yl)ethyl]isoindoline-1,3-dione (1.5 g, 4.1 mmol) in ethanol (20.0 mL, 343 mmol) and hydrazine (0.8 mL, 20 mmol). The mixture was allowed to stir at 85 C for 12 Hours. After being allowed to cool to ambient temperature the mixture was concentrated, affording a residue which was subjected to silica gel chromatography (eluent dichloromethane:methanol=1:1) to give 2-(5-chloro-4-nitro-1H-indol-3-yl)ethanamine (500 mg, 2.086 mmol, 51% yield) as yellow solid.

Step 4: A reaction tube under a nitrogen atmosphere was charged with a solution of 2-(5-chloro-4-nitro-1H-indol-3-yl)ethanamine (800 mg, 3.338 mmol) in trifluoroacetic acid (1.5 mL, 20 mmol) and 2-morpholinopyrimidine-5-carbaldehyde (2.0 g, 10 mmol). The mixture was allowed to stir at 150 C for 1 Hour in a Microwave reactor. The mixture was concentrated under reduced pressure to give a residue which was washed with water and adjust to pH=8 with an aq. Na₂CO₃ solution and then extracted with ethyl acetate (3*50 mL). The combined organic phase was concentrated to give a residue which was subjected to silica gel chromatography (dichloromethane:methanol=1:1) affording 4-[5-(6-chloro-5-nitro-2,3,4,9-tetrahydro-1H-pyrido[3,4-b]indol-1-yl)pyrimidin-2-yl]morpholine (1.0 g, 2.4 mmol, 72% yield) as yellow solid.

Step 5: A 50 mL single neck round-bottom flask was charged with a solution of 4-[5-(6-chloro-5-nitro-2,3,4,9-tetrahydro-1H-pyrido[3,4-b]indol-1-yl)pyrimidin-2-yl]morpholine (200 mg, 0.482 mmol) in tetrahydrofuran (10.0 mL), N,N-diisopropylethylamine (0.25 mL, 1.4 mmol) and 2-chloro-4,6-dimethyl-1,3,5-triazine (210 mg, 1.463 mmol). The mixture was allowed to stir at 70 C for 3 hours. The reaction mixture was concentrated to give a dark residue which was subjected to purification by silica gel chromatography (dichloromethane:methanol=10:1). Compound 325 (200 mg, 0.383 mmol, 79.5% yield) was isolated as yellow solid. MS m/z=521.9 [M+H]⁺; ¹H NMR (400 MHz, CD₃Cl) δ 8.41 (s, 1H), 8.34 (s, 2H), 7.34 (d, J=8.8 Hz, 1H), 7.24 (d, J=8.4 Hz, 1H), 7.10 (s, 1H), 5.13 (d, J=5.2 Hz, 1H), 3.76 (d, J=4.8 Hz, 4H), 3.72 (d, J=4.0 Hz, 4H), 3.14-3.18 (m, 1H), 2.87-2.89 (m, 1H), 2.69 (q, J=3.2 Hz, 1H), 2.42-2.48 (m, 6H).

Using the procedure described for Compound 325 above, additional compounds described herein were prepared by substituting the appropriate startings materials, suitable reagents, and reaction conditions, obtaining compounds such as those selected from:

| Cpd | Data |
|---|---|
| 266 | MS m/z = 522.1 [M − H]⁻; ¹H NMR (400 MHz, DMSO-d) δ |
| | 11.98 (s, 1H), 8.82 (d, J = 4.8 Hz, 1H), 7.64 (d, J = 8.7 Hz, 1H), |
| | 7.37 (d, J = 8.7 Hz, 2H), 7.18 (d, J = 4.8 Hz, 1H), 7.06 (t, J = |
| | 7.5 Hz, 1H), 6.84 (t, J = 7.4 Hz, 1H), 4.88 (d, J = 13.4 Hz, 1H), |
| | 3.46 3.42 (m, 1H), 2.76 (ddd, J = 16.7, 11.6, 5.7 Hz, 1H), 2.64- |
| | 2.57 (m, 1H), 2.28 (d, J = 1.9 Hz, 3H). |
| 326 | MS m/z = 560.9 [M + H]⁺; ¹H NMR (400 MHz, CDCl₃) δ 8.56 |
| | (d, J = 4.8 Hz, 1H), 8.37 (s, 3H), 7.34 (d, J = 8.8 Hz, 1H), 7.23 |
| | (d, J = 8.4 Hz, 1H), 6.99 (s, 1H), 6.84 (d, J = 4.8 Hz, 1H), 5.10 |
| | (q, J = 5.2 Hz, 1H), 3.77 (t, J = 4.4 Hz, 4H), 3.71 (t, J = |
| | 4.8 Hz, 4H), 3.24-3.32 (m, 1H), 2.87-2.95 (m, 1H), 2.71 (q, J = |
| | 3.2 Hz, 1H). |

### Example 51

### Preparation of Compound 327

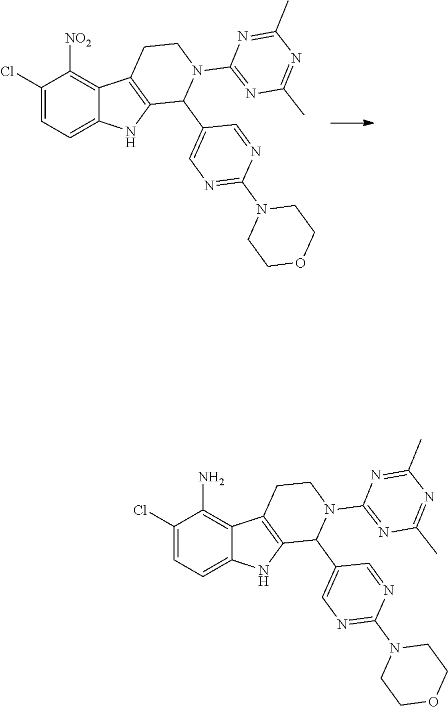 [SMILES: Cc1nc(C)nc(N2CCc3c([nH]c4ccc(Cl)c([N+](=O)[O-])c34)C2c2cnc(N3CCOCC3)nc2)n1 ; Cc1nc(C)nc(N2CCc3c([nH]c4ccc(Cl)c(N)c34)C2c2cnc(N3CCOCC3)nc2)n1]

A 100 mL single neck round-bottom flask was charged with a solution of 4-[5-[6-chloro-2-(4,6-dimethyl-1,3,5-triazin-2-yl)-5-nitro-1,3,4,9-tetrahydropyrido[3,4-b]indol-1-yl]pyrimidin-2-yl]morpholine (170 mg, 0.326 mmol) in tetrahydrofuran (10.0 mL), nickel (60 mg, 1.02 mmol) in water (5.0 mL) at 0 C then hydrazine (23.0 μL, 0.689 mmol) was added and the mixture was allowed to stir at ambient temperature for 1 Hour. The mixture was filtered and the filtrate concentrated under reduced pressure to give a dark residue which was purified by column chromatography (eluent dichloromethane:methanol=10:1). Compound 327 (120 mg, 0.244 mmol, 74.9% yield) was isolated as an off-white solid. MS m/z=492.0 [M+H]⁺, ¹H NMR (400 MHz, CD₃Cl) δ 8.35 (s, 2H), 7.95 (s, 1H), 7.01 (t, J=5.4 Hz, 2H), 6.62 (d, J=8.4 Hz, 1H), 5.13 (d, J=8.4 Hz, 1H), 4.35 (s, 2H), 3.75 (s, 4H), 3.73 (s, 4H), 3.12-3.23 (m, 3H), 2.49 (n, 6H).

Using the procedure described for Compound 327 above, additional compounds described herein were prepared by substituting the appropriate starting materials, suitable reagents, and reaction conditions, obtaining compounds such as those selected from:

| Cpd | Data |
|---|---|
| 273 | MS m/z = 494.1 [M + H]⁺; ¹H NMR (500 MHz, DMSO-d₆) δ 10.84 (s, 1H), 8.71 (d, |
| | J = 4.9 Hz, 1H), 7.47 (s, 1H), 7.14 (s, 1H), 7.09-6.86 (m, 3H), 6.71 (t, J = 7.3 Hz, |
| | 1H), 4.82 (s, 1H), 4.11 (br s, 2H), 3.31 (td, J = 13.1, 12.5, 4.2 Hz, 1H), 2.76 (ddd, |
| | J = 23.0, 13.3, 4.9 Hz, 2H), 2.19 (s, 3H). |
| 282 | MS m/z = 494.1 [M + H]⁺; HH NMR (500 MHz, DMSO-d₆) δ 10.43 (s, 1H), 8.70 |
| | (d, J = 4.9 Hz, 1H), 7.26 (s, 1H), 7.11 (s, 1H), 7.04 (d, J = 4.9 Hz, 1H), 6.95 (t, J = |
| | 7.4 Hz, 1H), 6.69 (t, J = 7.4 Hz, 1H), 6.66 (s, 1H), 4.86-4.84 (m, 3H), 2.93-2.61 |
| | (m, 2H), 2.19-2.17 (m, 4H). |
| 311 | MS m/z = 530.9 [M + H]⁺; ¹H NMR (400 MHz, CD₃Cl) δ 8.54 (d, J = 4.4 Hz, 1H), |
| | 8.36 (s, 2H), 7.67 (s, 1H), 7.42 (s, 1H), 6.86 (s, 1H), 6.79 (d, J = 4.8 Hz, 1H), 6.64 |
| | (s, 1H), 5.09 (dd, J = 4.0 Hz, 1H), 3.73 (d, J = 8.8 Hz, 8H), 3.27 (t, J = 10.4 Hz, |
| | 1H), 2.77-2.92 (m, 2H). |
| 320 | MS m/z = 492.0 [M + H]⁺; ¹H NMR (400 MHz, CD₃Cl) δ 8.37 (s, 2H), 7.49 (s, 1H), |
| | 7.42 (s, 1H), 6.99 (s, 1H), 6.67 (s, 1H), 5.14 (d, J = 4.0 Hz, 1H), 3.76 (d, J = 2.8 |
| | Hz, 4H), 3.73 (d, J = 4.4 Hz, 4H), 3.16-3.22 (m, 1H), 2.78-2.87 (m, 2H), 2.46 |
| | (d, J = 21.6 Hz, 6H). |
| 328 | MS m/z = 530, 9 [M + H]⁺; ¹H NMR (400 MHz, CDCl₃) δ 8.55 (d, J = 4.8 Hz, 1H), |
| | 8.38 (s, 2H), 7.76 (s, 1H), 7.02 (d, J = 8.8 Hz, 1H), 6.91 (s, 1H), 6.82 (d, J = 4.8 |
| | Hz, 1H), 6.63 (d, J = 8.8 Hz, 1H), 5.10 (q, J = 4.4 Hz, 1H), 4.36 (s, 2H), 3.76 (q, J = |
| | 3.4 Hz, 4H), 3.72 (q, J = 3.6 Hz, 4H), 3.14-3.36 (m, 3H). |

### Example 52

### Preparation of Compound 331

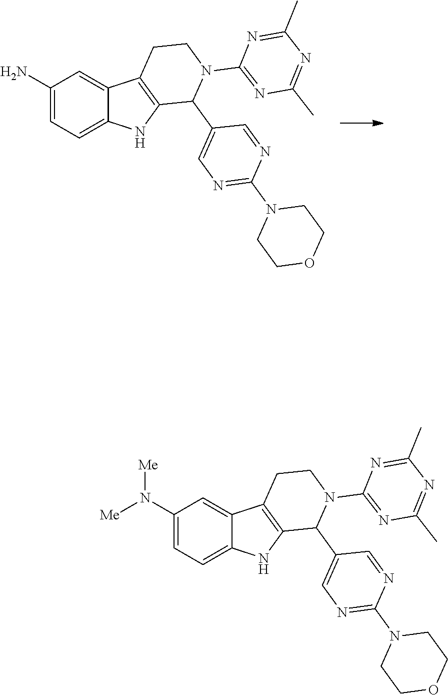 [SMILES: Cc1nc(C)nc(N2CCc3c([nH]c4ccc(N)cc34)C2c2cnc(N3CCOCC3)nc2)n1 ; Cc1nc(C)nc(N2CCc3c([nH]c4ccc(N(C)C)cc34)C2c2cnc(N3CCOCC3)nc2)n1]

A 20 mL vial was charged with 2-(4,6-dimethyl-1,3,5-triazin-2-yl)-1-(2-morpholinopyrimidin-5-yl)-1,3,4,9-tetrahydropyrido[3,4-b]indol-6-amine (100 mg, 0.219 mmol), paraformaldehyde (62 mg, 0.657 mmol), tetrahydrofuran (3 mL, 36.9 mmol) and sodium cyanoborohydride (36 mg, 0.573 mmol). The reaction mixture was allowed to stir for 2 Hours at ambient temperature after which the contents were concentrated under reduced pressure. The resulting residue was purified by prep-HPLC, affording Compound 331 (48 mg, 0.099 mmol, 45% yield). MS m/z=484.1 [M−H]⁻, ¹H NMR (DMSO-d₆, 400 MHz) δ 10.49 (s, 1H), 8.30 (s, 2H), 7.15-7.13 (d, J=8.8 Hz, 1H), 6.87 (s, 1H), 6.78-6.74 (m, 2H), 5.04-4.99 (dd, J=4.8 Hz, 13.2 Hz, 1H), 3.67-3.65 (m, 4H), 3.62-3.60 (m, 4H), 3.29-3.22 (m, 1H), 2.88-2.86 (m, 1H), 2.84 (s, 6H), 2.77-2.67 (m, 1H), 2.39 (s, 3H), 2.32 (s, 3H).

Using the procedure described for Compound 331 above, additional compounds described herein may be prepared by substituting the appropriate starting materials, suitable reagents, and reaction conditions, obtaining compounds such as those selected from:

| Cpd | Data |
|---|---|
| 329 | MS m/z = 523.1 [M − H]⁻; 1H (DMSO-d₆, 400 MHz) δ 8.78 (br |
| | s, 1H), 8.32 (s, 2H), 7.34-7.32 (d, J = 9.2 Hz, 1H), 7.12-7.11 (d, |
| | J = 4.8 Hz, 1H), 6.84-6.81 (m, 2H), 6.18 (s, 1H), 5.34-5.31 (d, |
| | J = 11.2 Hz, 1H), 4.93-4.90 (m, 1H), 3.64-3.64 (m, 4H), 3.62- |
| | 3.61 (m, 4H), 2.93-2.88 (m, 1H), 2.86 (m, 6H), 2.84-2.67 (s, 1H), |
| | 2.81-2.78 (m, 1H). |
| 330 | MS m/z = 486.3 [M + H]⁺; ¹H NMR (400 MHz, CDCl₃) δ 8.47- |
| | 8.46 (d, J = 4.8 Hz, 1H), 7.71 (brs, 1H), 7.1-7.12 (m, 2H), |
| | 6.92-6.85 (m, 2H), 6.77-6.73 (m, 3H), 5.09-5.05 (dd, J = 4.4, |
| | 13.6 Hz, 1H), 3.46-3.39 (dt, J = 4.0, 12.4 Hz, 1H), 2.94- |
| | 2.92 (m, 1H), 2.89 (s, 6H), 2.82-2.77 (m, 1H), 2.20-2.19 |
| | (d, J = 2.0 Hz, 3H). |

### Example 53

### Preparation of Compound 224

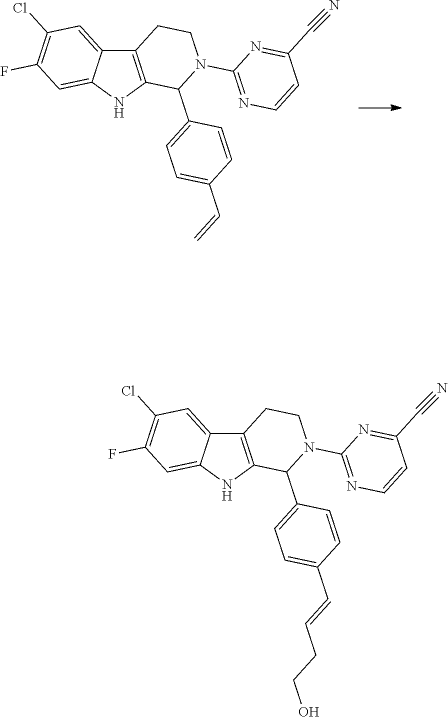 [SMILES: C=Cc1ccc(C2c3[nH]c4cc(F)c(Cl)cc4c3CCN2c2nccc(C#N)n2)cc1 ; N#Cc1ccnc(N2CCc3c([nH]c4cc(F)c(Cl)cc34)C2c2ccc(/C=C/CCO)cc2)n1]

A 100 mL 3-neck round-bottom flask under argon was charged with a solution of 2-[6-chloro-7-fluoro-1-(4-vinylphenyl)-1,3,4,9-tetrahydropyrido[3,4-b]indol-2-yl]pyrimidine-4-carbonitrile (200 mg, 0.4652 mmol) in dichloromethane (20 mL), but-3-en-1-ol (200 mg, 2.774 mmol) and Grubbs catalyst 2nd generation (40 mg, 0.047 mmol). The mixture was allowed to stir at 50 C for 4 days. The solution was concentrated, and the residue subjected to purification by prep-HPLC to afford Compound 224 (40 mg, 0.084 mmol, 18% yield) as a white solid. MS m/z=472.1 [M−H]⁻; ¹H NMR (DMSO-d₆, 400 MHz) δ 11.34 (s, 1H), 8.75 (s, 1H), 7.67 (d, J=7.4 Hz, 1H), 7.41-7.19 (m, 6H), 7.02 (s, 1H), 6.41 (d, J=16.0 Hz, J H), 6.35-6.20 (m, 1H), 4.82 (s, 1H), 4.59 (t, J=5.3 Hz, 1H), 3.56-3.42 (m, 2H), 3.27-3.17 (m, 1H), 2.91-2.73 (m, 2H), 2.37-2.22 (m, 2H).

Using the procedure described for Compound 224 above, additional compounds described herein were prepared by substituting the appropriate starting materials, suitable reagents, and reaction conditions, obtaining compounds such as those selected from:

| Cpd | Data |
|---|---|
| 167 | MS m/z = 517.1 [M + H]⁺; ¹H NMR (400 MHz, DMSO-d₆) δ 11.32 (s, 1H), 8.79 (s, |
| | 1H), 7.67 (d, J = 7.4 Hz, 1H), 7.48-7.20 (m, 5H), 7.19-6.97 (m, 2H), 6.40 (d, J = |
| | 16.0 Hz, 1H), 6.32-6.22 (m, 1H), 4.90 (s, 1H), 4.57 (t, J = 5.0 Hz, 1H), 3.49 (dd, |
| | J = 11.5, 6.4 Hz, 2H), 3.28-3.20 (m, 1H), 2.93-2.76 (m, 2H), 2.31 (dd, J = 13.3, 6.4 |
| | Hz, 2H). |
| 236 | MS m/z = 474.0 [M + H]⁺; ¹H NMR (400 MHz, DMSO-d₆) δ 11.54 (s, 1H), 8.76 (s, |
| | 1H), 7.38 (d, J = 8.0 Hz, 2H), 7.31-7.12 (m, 5H), 7.04 (s, 1H), 6.46-6.39(m, 1H), |
| | 6.31-6.24 (m, 1H), 4.82 (s, 1H), 4.60 (t, J = 4.0 Hz, 1H), 3.50 (q, J = 8.0, 2H), 3.30- |
| | 3.22(m, 1H), 3.00-2.99(m, 2H), 2.32 (q, J = 4.0 Hz, 2H). |
| 237 | MS m/z = 463.0 [M + H]⁻; ¹H NMR (400 MHz, DMSO-d₆) δ 11.53 (s, 1H), 8.30 (d, |
| | J = 4.8 Hz, 1H), 7.35(d, J = 8.0 Hz, 2H), 7.29-7.22(m, 2H), 7.19-7.09 (m, 3 H), 6.58 |
| | (d, J = 4.8 Hz, 1H), 6.42-6.38 (m, 1H), 6.30-6.22 (m, 1H), 4.88 (d, J = 11.6 Hz, |
| | 1H), 4.64-4.57 (m, 1H), 3.51-3.47(m, 2H), 3.19-3.10 (m, 1H), 2.98-2.88 (m, |
| | 2H), 2.37-2.27 (m, 5H). |
| 239 | MS m/z = 479.0 [M + H]⁻; ¹H NMR (DMSO-d₆, 400 MHz) δ 11.32 (s, 1H), 8.37(d, J = |
| | 1.6 Hz, 1H), 7.64 (d, J = 7.6 Hz, 1H), 7.36-7.30 (m, 3 H), 7.21 (d, J = 8.4 Hz, 2H), 7.02 |
| | (s, 1H), 6.40 (d, J = 16 Hz, 1H), 6.29-6.22 (m, 1H), 4.81-4.77 (m, 1H), 4.59 (t, |
| | J = 5.2 Hz, 1H), 3.49 (dd, J = 6.4 Hz, 12 Hz, 2H), 3.19-3.11 (m, 1H), 2.79-2.77 (m, |
| | 2H), 2.36 (d, J = 2.0 Hz, 3H), 2.31 (dd, J = 6.4 Hz, 13.2 Hz, 2H). |
| 241 | MS m/z = 479.0 [M + H]⁻; ¹H NMR (400 MHz, DMSO-d₆) δ 11.54 (s, 1H), 8.37 (d, J = |
| | 4.0 Hz, 1H), 7.35 (d, J = 8.0 Hz, 2H), 7.23 (d, J = 8.0 Hz, 2H), 7.17-7.10(m, 2H), 7.04 |
| | (s, 1H), 6.40 (d, J = 16.0 Hz, 1H), 6.30-6.22(m, 1H), 4.80-4.76(m, 1H), 4.60 (t, J = |
| | 8.0 Hz, 1H), 3.49 (q, J = 8.0 Hz, 2H), 3.22-3.15(m, 1H), 3.00-2.91(m, 2H), 2.37 (d, |
| | J = 2.0 Hz, 3H), 2.34-2.29 (m, 2H). |
| 242 | MS m/z = 475.0 [M + H]⁻; ¹H NMR (400 MHz, DMSO-d₆) δ 11.50 (s, 1H), 7.34 (d, |
| | J = 8.0 Hz, 2H), 7.22 (d, J = 8.4 Hz, 2H), 7.18 (s, 1H), 7.16-7.09 (m, 2H), 6.47 (s, |
| | 1H), 6.41-6.38 (m, 1H), 6.29-6.20 (m, 1H), 4.94-4.86 (m, 1H), 4.58 (t, J = 5.2 Hz, |
| | 1H), 3.51-3.46 (m, 2H), 3.17-3.07(m, 1H), 2.96-2.89 (m, 2H), 2.33-2.25 (m, |
| | 8H). |
| 243 | MS m/z = 493.0 [M + H]⁻; ¹H NMR (400 MHz, DMSO-d₆) δ 11.50 (s, 1H), 7.35 (d, |
| | J = 8.0 Hz, 2H), 7.22 (d, J = 8.0 Hz, 2H), 7.16-7.09 (m, 2H), 7.04 (s, 1H), 6.42- |
| | 6.38 (m, 1H), 6.29-6.22 (m, 1H), 4.82-4.74 (m, 1H), 4.58 (t, J = 5.2 Hz, 1H), 3.51- |
| | 3.46 (m, 2H), 3.19-3.09 (m, 1H), 3.00-2.86 (m, 2H), 2.35-2.27 (m, 8H). |
| 244 | MS m/z = 477.0 [M + H]⁺; ¹H NMR (400 MHz, DMSO-d₆) δ 11.31 (s, 1H), 7.63 (d, |
| | J = 7.4 Hz, 1H), 7.38-7.27 (m, 3H), 7.26-7.13 (m, 3H), 6.46 (s, 1H), 6.40 (d, J = 16.0 |
| | Hz, 1H), 6.29-6.20 (m, 1H), 4.94-4.87 (m, 1H), 4.58 (t, J = 5.3 Hz, 1H), 3.49 (dd, J = |
| | 12.0, 6.6 Hz, 2H), 3.13-3.05 (m, 1H), 2.81-2.72 (m, 2H), 2.41-2.17 (m, 8H). |

### Example 54

### Preparation of Compound 240

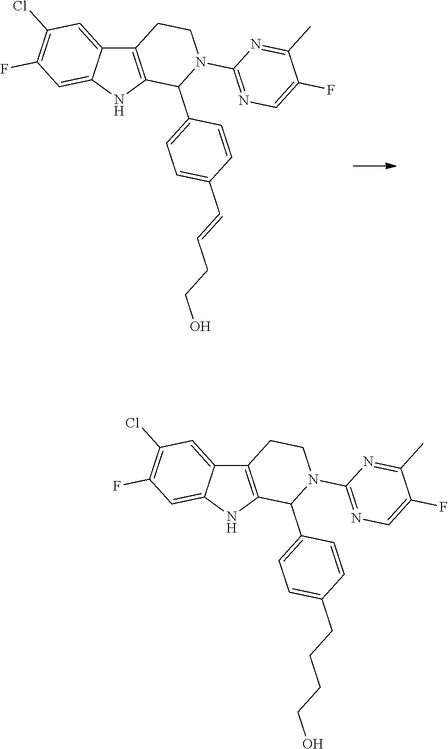 [SMILES: Cc1nc(N2CCc3c([nH]c4cc(F)c(Cl)cc34)C2c2ccc(/C=C/CCO)cc2)ncc1F ; Cc1nc(N2CCc3c([nH]c4cc(F)c(Cl)cc34)C2c2ccc(CCCCO)cc2)ncc1F]

A 100 mL 2-neck round-bottom flask equipped with a stir bar, under argon, was charged with (E)-4-[4-[6-chloro-7-fluoro-2-(5-fluoro-4-methyl-pyrimidin-2-yl)-1,3,4,9-tetrahydropyrido[3,4-b]indol-1-yl]phenyl]but-3-en-1-ol (30 mg, 0.062 mmol), ethyl acetate (0.5 mL) and platinum (IV) oxide (2 mg, 0.009 mmol). The atmosphere was exchanged with hydrogen (1 atm) and the reaction mixture allowed to stir at ambient temperature for 3 hours. Subsequently the reaction mixture was filtered through a pad of Celite, concentrated under reduced pressure and the resulting residue subjected to purification by prep-TLC (ethylacetate:petrol ether=1:3) to afford Compound 240 (25 mg, 0.052 mmol, 83% yield) as a white solid. MS m/z=481.0 [M−H]⁻; ¹H NMR (DMSO-d₆, 400 MHz) δ 11.31 (s, 1H), 8.37 (s, 1H), 7.64 (d, J=7.2 Hz, 1H), 7.30 (d, J=10 Hz, 1H), 7.18 (dd, J=8.0 Hz, 15.6 Hz, 4H), 7.03 (s, 1H), 4.81-4.77 (m, 1H), 4.37 (t, J=4.8 Hz, 1H), 3.38 (dd, J=6.0 Hz, 12 Hz, 2H), 3.19-3.12 (m, 1H), 2.79-2.77 (m, 2H), 2.54 (d, J=8.0 Hz, 2H), 2.36 (d, J=1.6 Hz, 3H), 1.58-1.49 (m, 2H), 1.44-1.37 (m, 2H).

Using the procedure described for Compound 240 above, additional compounds described herein were prepared by substituting the appropriate starting materials, suitable reagents, and reaction conditions obtaining compounds such as those selected from:

| Cpd | Data |
|---|---|
| 168 | MS m/z = 519.2, [M + H]⁺; ¹H NMR (400 MHz, DMSO-d₆) δ 11.33 (s, 1H), 8.78 (s, 1H), |
| | 7.67 (d, J = 7.3 Hz, 1H), 7.32 (d, J = 10.1 Hz, 1H), 7.20 (dd, J = 19.8, 8.2 Hz, 4H), 7.13- |
| | 6.97 (m, 2H), 4.91 (s, 1H), 4.37 (t, J = 5.1 Hz, 1H), 3.44-3.36 (m, 2H), 3.30-3.21 (m, |
| | 1H), 2.92-2.74 (m, 2H), 2.55 (d, J = 7.5 Hz, 2H), 1.60-1.47 (m, 2H), 1.45-1.35 (m, |
| | 2H). |
| 245 | MS m/z = 479.0, [M + H]⁺; ¹H NMR (400 MHz, DMSO-d₆) δ 11.30 (s, 1H), 7.63 (d, J = |
| | 7.4 Hz, 1H), 7.35-7.27 (m, 1H), 7.24-7.09 (m, 5H), 6.45 (s, 1H), 4.91 (d, J = 12.0 Hz, |
| | 1H), 4.37 (t, J = 5.1 Hz, 1H), 3.38 (dd, J = 11.8, 6.5 Hz, 2H), 3.15-3.04 (m, 1H), 2.82-2.71 |
| | (m, 2H), 2.54 (d, J = 7.5 Hz, 2H), 2.28 (s, 6H), 1.60-1.48 (m, 2H), 1.45-1.35 (m, 2H). |
| 246 | MS m/z = 481.0, [M − H]⁻; ¹H NMR (400 MHz, DMSO-d₆) δ 11.51 (s, 1H), 8.37 (d, J = |
| | 2.0 Hz, 1H), 7.22-7.10(m, 6H), 7.05 (s, 1H), 4.80-4.76(m, 1H), 4.37 (t, J = 4.0 Hz, 1H), |
| | 3.40-3.36(m, 2H), 3.22-3.15 (m, 1H), 3.01-2.91(m, 2H), 2.56-2.54 (m, 2H), 2.37 (d, J = |
| | 4.0 Hz, 3H), 1.57-1.53(m, 2H), 1.42-1.38(m, 2H). |
| 249 | MS m/z = 495.0, [M + H]⁺; ¹H NMR (400 MHz, DMSO-d₆) δ 11.29 (s, 1H), 7.63 (d, |
| | J = 7.2 Hz, 1H), 7.29 (d, J = 10 Hz, 1H), 7.20-7.14 (m, 4 H), 7.04 (s, 1H), 4.79 (d, J = 12.8 |
| | Hz, 1H), 4.38-4.36 (m, 1H), 3.38 (d, J = 6 Hz, 2H), 3.13-3.10 (m, 1H), 2.77 (s, 2H), 2.50 |
| | (s, 2H), 2.32 (s, 6H), 1.56-1.50 (m, 2H), 1.42-1.38 (m, 2H) |

### Example 55

### Preparation of Compound 223

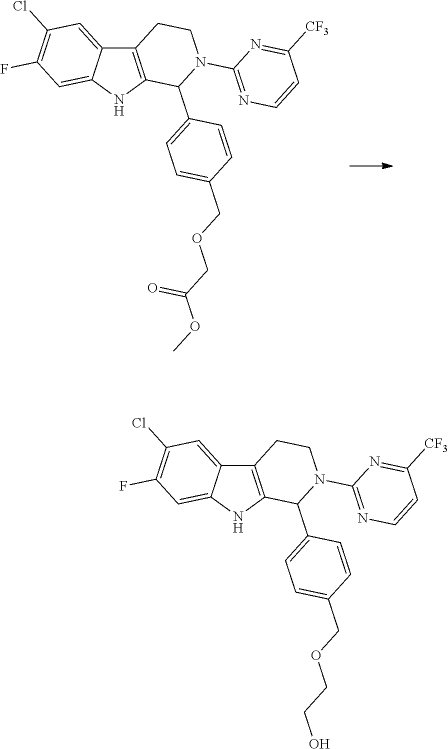 [SMILES: COC(=O)COCc1ccc(C2c3[nH]c4cc(F)c(Cl)cc4c3CCN2c2nccc(C(F)(F)F)n2)cc1 ; OCCOCc1ccc(C2c3[nH]c4cc(F)c(Cl)cc4c3CCN2c2nccc(C(F)(F)F)n2)cc1]

A 20 mL vial equipped with a septum cap and a magnetic stir bar under an argon atmosphere was charged with a solution of methyl 2-[[4-[6-chloro-7-fluoro-2-[4-(trifluoromethyl)pyrimidin-2-yl]-1,3,4,9-tetrahydropyrido[3,4-b]indol-1-yl]phenyl]methoxy]acetate (130 mg, 0.237 mmol) in tetrahydrofuran (2 mL, 24.6 mmol). The vial was placed into a water ice bath, followed by addition of lithium aluminum hydride (15 mg, 0.395 mmol) at 0° C. The mixture was allowed to warm to 25° C. and allowed to stir for 2H. Subsequently the reaction was terminated by addition of ammonium chloride The reaction mixture was concentrated under reduced pressure. The solid was purified by prep-HPLC to give Compound 223 (57 mg, 0.109 mmol, 46% yield) as white solid. MS m/z=520.9, [M+H]⁺; ¹H NMR (DMSO-d₆, 400 MHz) δ 11.33 (s, 1H), 8.79 (s, 1H), 7.68 (d, J=7.6 Hz, 1H), 7.31 (dd, J=7.5 Hz, 5H), 7.11 (d, J=5.2 Hz, 2H), 4.90 (br, 1H), 4.63 (t, J=5.4 Hz, 1H), 4.46 (s, 2H), 3.51 (dd, J=5.2 Hz, 2H), 3.43 (t, J=5.2 Hz, 2H), 3.28 (s, 1H), 2.79-2.92 (m, 2H).

### Example 56

### Preparation of Compound 247

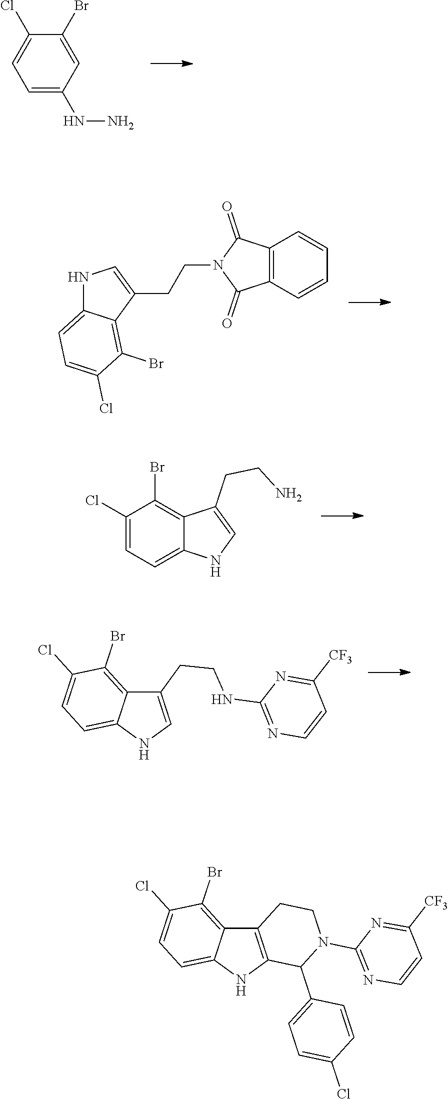 [SMILES: NNc1ccc(Cl)c(Br)c1 ; O=C1c2ccccc2C(=O)N1CCc1c[nH]c2ccc(Cl)c(Br)c12 ; NCCc1c[nH]c2ccc(Cl)c(Br)c12 ; FC(F)(F)c1ccnc(NCCc2c[nH]c3ccc(Cl)c(Br)c23)n1 ; FC(F)(F)c1ccnc(N2CCc3c([nH]c4ccc(Cl)c(Br)c34)C2c2ccc(Cl)cc2)n1]

Step 1: In a RBF, (3-bromo-4-chloro-phenyl)hydrazine hydrochloride (2.0 g, 7.8 mmol) and 2-(4,4-diethoxybutyl)isoindoline-1,3-dione (2.4 g, 8.2 mmol) in 6 mL of EtOH was heated to 60° C. for 1 h in the presence of a small amount of water (0.1 mL). After addition of 0.5 mL of concentrated HCl solution, the mixture was heated to reflux for 14 h. LCMS showed complete consumption of starting material, and desired product could be detected. The mixture was cooled to rt, filtered, the filtration was treated with saturated aq.NaHCO₃ to adjust to pH 8, extracted with ethyl acetate three times. The extracts were combined and concentrated in vacuum, then further purified by silica gel column to obtain 2-[2-(4-bromo-5-chloro-1H-indol-3-yl)ethyl]isoindoline-1,3-dione (800 mg, 1.982 mmol, 26% yield) and 2-[2-(6-bromo-5-chloro-1H-indol-3-yl)ethyl]isoindoline-1,3-dione (800 mg, 1.982 mmol, 26% yield) as a yellow solid.

Step 2: Compound 2-[2-(4-bromo-5-chloro-1H-indol-3-yl)ethyl]isoindoline-1,3-dione (140 mg, 0.3468 mmol) was then treated with hydrazine (80.0 mg, 2.00 mmol) in the presence of 8 mL of EtOH (2.00 mL, 34.3 mmol) and 2 mL of H₂O at RT for 15 h. The volatiles were removed by reduced pressure, and the residue was extracted with CH₂Cl₂ (20 mL*3). The combined organic layers were washed with H₂O, dried, filtered, and then concentrated to a residue that was purified by flash chromatography using 15% methanol, 1% NH₄OH in CH₂Cl₂ to give 2-(4-bromo-5-chloro-1H-indol-3-yl)ethanamine (75 mg, 0.27416 mmol, 79.06% yield).

Step 3: 2-(4-bromo-5-chloro-1H-indol-3-yl)ethanamine (100 mg, 0.36555 mmol) was dissolved in 1-butanol (2.00 mL, 21.8 mmol), then 2-chloro-4-(trifluoromethyl)pyrimidine (80 mg, 0.43828 mmol), triethylamine (75 mg, 0.74118 mmol) were added. The mixture was stirred at 120° C. for 15 h. LCMS showed most of the starting material was consumed, then the organic layer was concentrated via rotovap. The product was purified via flash chromatography (silica): the crude mixture was dissolved in a minimum volume of EtOAc, then added to the column and eluted with EtOAc/Hexane to give the target compound N-[2-(4-bromo-5-chloro-1H-indol-3-yl)ethyl]-4-(trifluoromethyl)pyrimidin-2-amine as a yellow solid

Step 4: A 3-neck round-bottom flask, equipped with a nitrogen inlet, was charged with N-[2-(4-bromo-5-chloro-1H-indol-3-yl)ethyl]-4-(trifluoromethyl)pyrimidin-2-amine (100 mg, 0.238 mmol), 4-chlorobenzaldehyde (40 mg, 0.285 mmol), p-toluenesulfonic acid (25 mg, 0.144 mmol) and the solids suspended in 1-butanol (2 mL). The reaction mixture was allowed to stir at 120° C. for 14 h. After being allowed to cool to ambient temperature, the reaction mixture was concentrated in vacuo. After purification by pre-HPLC, Compound 247 (100 mg, 0.184 mmol, 77% yield) was obtained as a light yellow solid. MS m/z=541.0 [M+H]⁺; ¹H NMR (DMSO-de, 400 MHz) δ 11.60 (s, 1H), 8.80 (d, J=8.0 Hz, 1H), 7.44 (d, J=8.0 Hz, 2H), 7.37-7.34 (m, 3H), 7.25 (d, J=8.0 Hz, 1H), 7.14 (d, J=8.0 Hz, 1H), 7.08 (s, 1H), 4.93-4.82 (m, 1H), 3.38-3.34 (m, 1H), 3.28-3.22 (m, 1H), 3.11-2.03 (m, 1H).

### Example 57

### Preparation of Compound 322

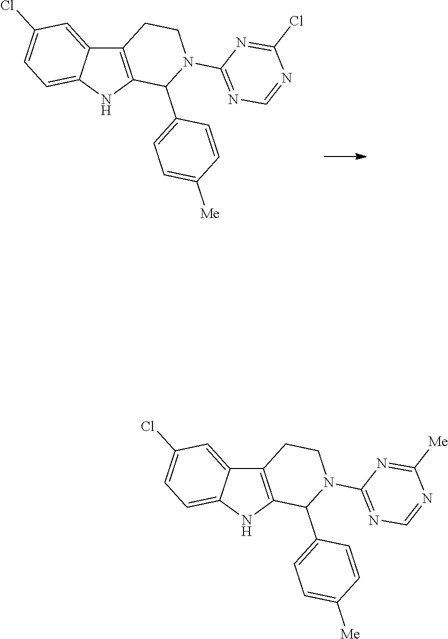 [SMILES: Cc1ccc(C2c3[nH]c4ccc(Cl)cc4c3CCN2c2ncnc(Cl)n2)cc1 ; Cc1ccc(C2c3[nH]c4ccc(Cl)cc4c3CCN2c2ncnc(C)n2)cc1]

A 3-neck round-bottom flask equipped with a stir bar was placed under a under nitrogen atmosphere and charged with 6-chloro-2-(4-chloro-1,3,5-triazin-2-yl)-1-(p-tolyl)-1,3,4,9-tetrahydropyrido[3,4-b]indole (150 mg, 0.366 mmol), ferric acetylacetonate (50 mg, 0.137 mmol) and tetrahydrofuran (3 mL, 37 mmol). The reaction mixture was placed in an acetone-dry ice bath and allowed to stir at −78° C. Then methylmagnesium chloride in tetrahydrofuran (0.3 mL, 0.9 mmol, 3 mol/L) was added dropwise and the reaction mixture was allowed to stir at 0° C. for 2 h, then allowed to stir at ambient temperature for 16 h. Sat. aq. NH₄Cl was added and the mixture extracted with ethyl acetate (3×20 mL). The combined organic phases were concentrated and the obtained residue, purified by prep-HPLC affording Compound 322 (35 mg, 0.090 mmol, 25% yield) as white solid. MS m/z=390.0 [M+H]⁺; ¹H NMR (DMSO-d₆, 400 MHz) δ 11.23 (d, J=3.2 Hz, 1H), 8.57 (d, J=23.6 Hz, 1H), 7.52 (d, J=2.0 Hz, 1H), 7.32 (d, J=8.8 Hz, 1H), 7.18-7.11 (m, 5H), 7.08 (dd, J=8.4, 2.0 Hz, 1H), 4.94-4.86 (m, 1H), 3.19-3.14 (m, 1H), 2.90-2.75 (m, 2H), 2.40 (d, J=4.0 Hz, 3H), 2.27 (s, 3H).

Using the procedure described for Compound 322 above, additional compounds described herein were prepared by substituting the appropriate starting materials, suitable reagents, and reaction conditions, obtaining compounds such as those selected from:

| Cpd | Data |
|---|---|
| 294 | MS m/z = 463.2, [M + H]⁺; ¹H NMR (500 MHz, DMSO-d₆) δ |
| | 11.05 (d, J = 3.7 Hz, 1H), 8.51 (d, J = 27.0 Hz, 1H), 8.26 (d, |
| | J = 8.9 Hz, 2H), 7.47 (d, J = 2.0 Hz, 1H), 7.25 (d, J = |
| | 8.5 Hz, 1H), 7.02 (dd, J = 8.6, 2.1 Hz, 1H), 6.83 (d, J = 13.9 |
| | Hz, 1H), 4.94 (ddd, J = 19.7, 13.4, 5.2 Hz, 1H), 3.57 (dd, J = |
| | 21.4, 5.0 Hz, 8H), 3.24-3.21 (m, 1H), 2.86 (dd, J = 15.4, 3.9 Hz, |
| | 1H), 2.70 (td, J = 15.8, 14.0, 5.5 Hz, 1H), 2.34 (d, J = 32.0 |
| | Hz, 3H). |

### Example 58

### Preparation of Compound 384

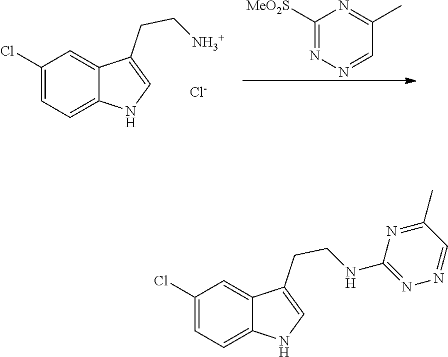 [SMILES: [NH3+]CCc1c[nH]c2ccc(Cl)cc12 ; [Cl-] ; Cc1cnnc(S(C)(=O)=O)n1 ; Cc1cnnc(NCCc2c[nH]c3ccc(Cl)cc23)n1]

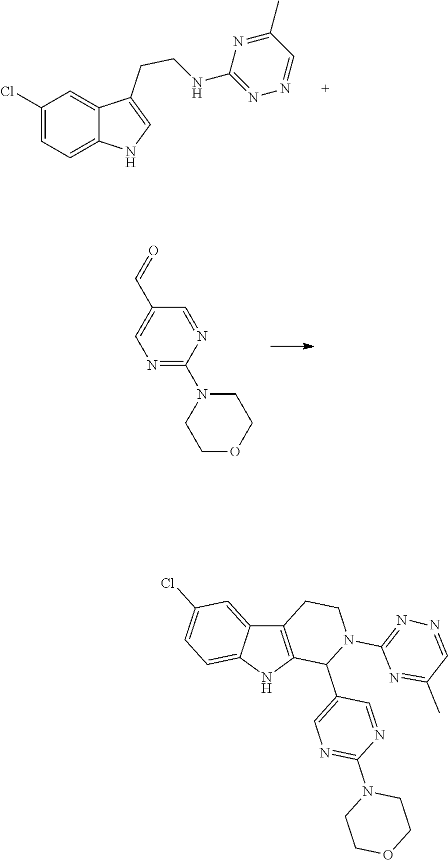 [SMILES: Cc1cnnc(NCCc2c[nH]c3ccc(Cl)cc23)n1 ; O=Cc1cnc(N2CCOCC2)nc1 ; Cc1cnnc(N2CCc3c([nH]c4ccc(Cl)cc34)C2c2cnc(N3CCOCC3)nc2)n1]

Step 1: 5-chlorotryptamine hydrochloride (340 mg, 1.5 mmol) and 5-methyl-3-methylsulfonyl-1,2,4-triazine (256 mg, 1.5 mmol) were placed in a vial and dissolved in 10 mL of dioxane. Hunig's base (0.54 mL, 3.1 mmol) was added to the mixture which was then heated to 80° C. with stirring for 12 Hours. The solution was concentrated in vacuo and the crude material was purified by silica gel chromatography to provide N-[2-(5-chloro-1H-indol-3-yl)ethyl]-5-methyl-1,2,4-triazin-3-amine (256 mg, 0.87 mmol, 57% yield) as a white solid.

Step 2: Placed N-[2-(5-chloro-1H-indol-3-yl)ethyl]-5-methyl-1,2,4-triazin-3-amine (250 mg, 0.87 mmol), 2-morpholinopyrimidine-5-carbaldehyde (176 mg, 0.91 mmol) and p-toluene sulfonic acid (76 mg, 0.43 mmol) in a vial, sealed and flushed with Ar. Dissolved in 10 mL of 2-BuOH and heated to 100° C. for 18 hours. Then cooled to RT, concentrated in vacuo and purified by silica gel chromatography to provide Compound 384 as a white solid, (120 mg, 0.26 mmol, 30% yield). MS m/z=463.3, [M+H]⁺; ¹H NMR (400 MHz, DMSO-d₆) δ 11.23-10.83 (m, 1H), 8.64 (t, J=3.7 Hz, 1H), 8.35 (t, J=3.5 Hz, 2H), 7.55 (d, J=4.6 Hz, 1H), 7.40-7.24 (m, 1H), 7.10 (t, J=6.9 Hz, 1H), 6.92 (s, 1H), 5.05 (d, J=13.3 Hz, 1H), 4.04-3.50 (m, 8H), 3.40 (t, J=11.7 Hz, 1H), 2.89 (dd, J=38.6, 14.1 Hz, 2H), 2.41 (s, 3H).

### Example 59

### Preparation of Compound 383

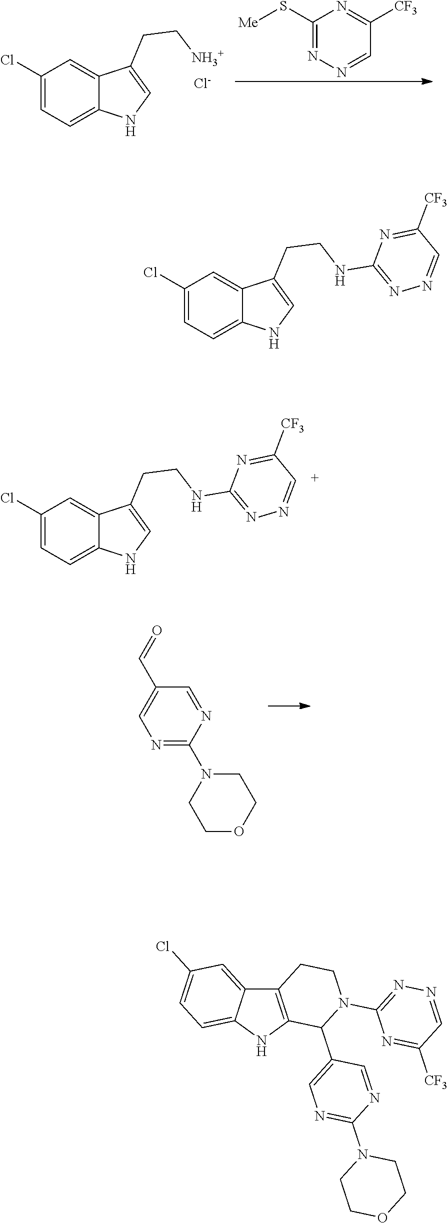

Step 1: 5-chlorotryptamine hydrochloride (2 g, 8.7 mmol) was added to vial containing 3-methylsulfanyl-5-(trifluoromethyl)-1,2,4-triazine (100 mg, 0.5 mmol %) and dissolved in 5 mL of n-BuOH, then added Hunig's base (0.22 mL, 1.3 mmol) was added to the mixture. The vial was sealed, flushed with Ar and heated to 120° C. for 18 hours. The mixture was cooled to RT and concentrated in vacuo. The crude material was purified by silica gel chromatography to provide N-[2-(5-chloro-1H-indol-3-yl)ethyl]-5-(trifluoromethyl)-1,2,4-triazin-3-amine (111 mg, 0.3 mmol, 63% yield) as a white solid.

Step 2: N-[2-(5-chloro-1H-indol-3-yl)ethyl]-5-(trifluoromethyl)-1,2,4-triazin-3-amine (111 mg, 0.32 mmol) 2-morpholinopyrimidine-5-carbaldehyde (65 mg, 0.34 mmol) and p-toluenesulfonic acid (28 mg, 0.16 mmol) in a vial, flushed with Ar and dissolved in 3 mL of 2-BuOH. Stirred at 100° C. for 18 hours, then cooled to RT, concentrated in vacuo and precipitated from methanol. Obtained Compound 383 (45 mg, 0.09 mmol, 27% yield) as a white solid. MS m/z=517.1 [M+H]⁺; ¹H NMR (400 MHz, DMSO-d₆) δ 11.11 (s, 1H), 9.23 (t, J=3.6 Hz, 1H), 8.36 (d, J=3.9 Hz, 2H), 7.58 (d, J=4.4 Hz, 1H), 7.36-7.17 (m, 1H), 7.10 (d, J=8.6 Hz, 1H), 6.79 (br s, 1H), 3.88-35.09, (br s, 1H). 49 (m, 9H), 3.09-2.77 (m, 2H).

Using the procedure described for Compound 383 above, additional compounds described herein were prepared by substituting the appropriate starting materials, suitable reagents, and reaction conditions, obtaining compounds such as those selected from:

| Cpd | Data |
|---|---|
| 403 | MS m/z = 480.0 [M + H]⁺. |

### Example 60

### Preparation of Compound 538

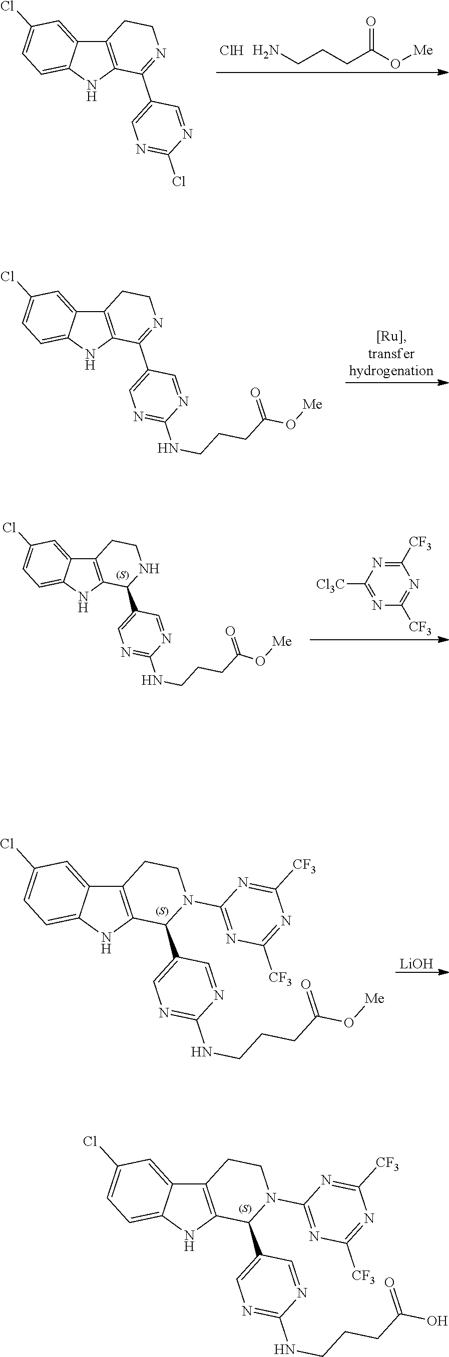

Step 1. Into a 50 mL RBF, was added: 6-chloro-1-(2-chloropyrimidin-5-yl)-4,9-dihydro-3H-pyrido[3,4-b]indole (500 mg, 1.58 mmol) in acetonitrile (10 mL) and methyl 4-aminobutanoate hydrochloride (400 mg, 2.6 mmol) and Hunig's base (0.9 mL, 5 mmol). The mixture was stirred at 50° C. for 5 h, diluted with ethyl acetate (50 mL), washed with water (20 mL*3) and brine (20 mL*2), dried over anhydrous sodium sulfate, then filtered and concentrated in vacuo. The residue was purified by flash chromatography (eluent: DCM/NH₃ in MeOH=90/10) to give methyl 4-[[5-(6-chloro-4,9-dihydro-3H-pyrido[3,4-b]indol-1-yl)pyrimidin-2-yl]amino]butanoate (450 mg, 1.13 mmol, 71.7% yield) as a white solid material.

Step 2. Into a 50 mL RBF, equipped with a N₂ inlet, was added: methyl 4-[[5-(6-chloro-4,9-dihydro-3H-pyrido[3,4-b]indol-1-yl)pyrimidin-2-yl]amino]butanoate (450 mg, 1.13 mmol), a formic acid triethylamine complex 5:2 (1.0 mL, 2.4 mmol) and acetonitrile (25 mL) at 25° C. RuCl[(R,R)-TsDPEN](mesitylene) (14 mg, 0.02 mmol) was added and the mixture was stirred for 16 h. The mixture was quenched with Sat. aqueous solution of NaHCO₃ and extracted with ethyl acetate, washed with brine, dried over anhydrous sodium sulfate and filtered, then concentrated in vacuo. The residue was purified by flash chromatography (eluent: DCM/NH₃ in MeOH=92/8) to give methyl 4-[[5-[(1S)-6-chloro-2,3,4,9-tetrahydro-1H-pyrido[3,4-b]indol-1-yl]pyrimidin-2-yl]amino]butanoate (330 mg, 0.82 mmol, 73% yield) as a brown solid material.

Step 3. Into a 50 mL RBF, was added: a solution of methyl 4-[[5-[(1S)-6-chloro-2,3,4,9-tetrahydro-1H-pyrido[3,4-b]indol-1-yl]pyrimidin-2-yl]amino]butanoate (330 mg, 0.83 mmol) in acetonitrile (20 mL), 2-(trichloromethyl)-4,6-bis(trifluoromethyl)-1,3,5-triazine (331 mg, 0.99 mmol) and DMAP (100 mg, 0.82 mmol). The mixture was stirred at RT for 3 h. The mixture was concentrated in vacuo and the residue was purified by flash chromatography (eluent: PE/EA=75/25) to give methyl 4-[[5-[(1S)-2-[4,6-bis(trifluoromethyl)-1,3,5-triazin-2-yl]-6-chloro-1,3,4,9-tetrahydropyrido[3,4-b]indol-1-yl]pyrimidin-2-yl]amino]butanoate (350 mg, 0.57 mmol, 69% yield) as a white solid material.

Step 4. Into a 25 mL RBF, was added: methyl 4-[[5-[(1S)-2-[4,6-bis(trifluoromethyl)-1,3,5-triazin-2-yl]-6-chloro-1,3,4,9-tetrahydropyrido[3,4-b]indol-1-yl]pyrimidin-2-yl]amino]butanoate (200 mg, 0.33 mmol), water (2 mL) THE (2 mL) and Lithium hydroxide monohydrate (34 mg, 0.81 mmol) at RT. The mixture was stirred for 3 h. To the mixture was added 1N HCl to adjust to pH=6-7 and extracted with ethyl acetate (10 mL*3). The combined organic phases were washed with brine, dried over anhydrous sodium sulfate, filtered and concentrated in vacuo. The residue was purified by Prep-HPLC to give Compound 538 (120 mg, 0.20 mmol, 61% yield) as a white solid material. MS m/z=600.8 [M+H]⁺; ¹H NMR (DMSO-d₆) δ 11.13 (s, 1H), 8.20 (s, 2H), 7.57 (d, J=2.4 Hz, 1H), 7.48 (t, J=5.6 Hz, 1H), 7.34 (d, J=8.8 Hz, 1H), 7.10 (dd, J=8.8, 2.0 Hz, 1H), 6.81 (s, 1H), 4.95 (dd, J=13.2, 5.2 Hz, 1H), 3.49-3.56 (m, 1H), 3.24 (q, J=13.0, 6.8 Hz, 2H), 3.02 (dd, J=15.2, 3.2 Hz, 1H), 2.80-2.88 (m, 1H), 2.23 (t, J=7.2 Hz, 2H), 1.66-1.73 (m, 2H).

Using the procedure described for Compound 538 above, additional compounds described herein were prepared by substituting the appropriate starting materials, suitable reagents, and reaction conditions, obtaining compounds such as those selected from:

| Cpd | Data |
|---|---|
| 537 | MS m/z = 586.9, [M + H]⁺; ¹H NMR (400 MHz, DMSO) δ 11.14 |
| | (s, 1H), 8.23 (s, 2H), 7.57 (d, J = 2.0 Hz, 1H), 7.45 (t, J = 5.6 |
| | Hz, 1H), 7.34 (d, J = 8.8 Hz, 1H), 7.10 (dd, J = 8.8, 2.4 Hz, 1H), |
| | 6.82 (s, 1H), 4.96 (dd, J = 13.4, 4.8 Hz, 1H), 3.49-3.57 (m, 1H), |
| | 3.43 (q, J = 12.6, 6.8 Hz, 2H), 3.03 (dd, J = 15.8, 3.2 Hz, 1H), |
| | 2.80-2.88 (m, 1H), 2.46 (t, J = 7.0 Hz, 2H). |
| 539 | MS m/z = 616.9, [M + H]⁺; ¹H NMR (400 MHz, DMSO) δ |
| | 11.16 (s, 1H), 8.21 (s, 2H), 7.57-7.59 (m, 2H), 7.34 (d, J = 8.4 Hz, |
| | 1H), 7.10 (dd, J = 9.0, 2.0 Hz, 1H), 6.82 (s, 1H), 4.96 (dd, J = |
| | 13.2, 4.8 Hz, 1H), 3.90 (s, 2H), 3.50-3.55 (m, 3H), 3.39 (dd, J = |
| | 11.4, 5.6 Hz, 2H), 3.00-3.05 (m, 1H), 2.80-2.88 (m, 1H). |

### BIOLOGICAL EXAMPLES

The following in vitro biological examples demonstrate the usefulness of the compounds of the present description as DHODH inhibitors.

To describe in more detail and assist in understanding the present description, the following biological examples are not to be construed as limiting the scope of the present description but are offered to illustrate the scope thereof. Such variations of the present description that may be now known or later developed, which would be within the purview of one skilled in the art to ascertain, are considered to fall within the scope of the present description and as hereinafter claimed.

### Example 61

### MOLM-13 Cell Proliferation Assay

The following assay was conducted to measure proliferation of MOLM-13 cells in the presence of each compound studied. This assay was included as an indicator of DHODH inhibition, as growth of MOLM-13 cells is known to depend on synthesis of pyrimidines using the de novo pathway, which can be blocked by inhibition of DHODH.
1. MOLM-13 cells were cultured in RPMI 1640 medium+10% Fetal Bovine Serum (FBS). Only low passage cells (<20) were used for the compound tests
2. On day 1, Log-phase growing Molm-13 Cells were counted and diluted to 10⁵ cells/mL in RPMI 1640 medium+10% FBS
3. 100 μL of diluted MOLM-13 cells in 96 well white plates were seeded, for a final density of 10⁴ cells/well.
4. 0.5 μL of pre-diluted 200× compounds or DMSO control was added to each well. The final DMSO concentration was 0.5%.
5. After 72 Hours of culture at 37° C., 5% CO₂, the plates were taken out and cooled to RT, then 50 μL CellTiter-Glo® (Promega) was added, and the composition was mixed at RT for 10 minutes.
6. The plates were then read by ViewLux (Speed-fast, Gain-High, Bining-1X, 5 seconds).

As shown in Table 1, the IC₅₀ (nM) for inhibition of proliferation was calculated for each compound (Cpd).

TABLE 1

| Cpd | IC₅₀ |
|---|---|
| 1 | 31.8 |
| 2 | 60.6 |
| 3 | 29.6 |
| 4 | 59.7 |
| 5 | 67.9 |
| 6 | 37.6 |
| 7 | 0.6 |
| 8 | 34.8 |
| 9 | 35.9 |
| 10 | 4.1 |
| 11 | 35.4 |
| 12 | 35.8 |
| 13 | 12.4 |
| 14 | 30.2 |
| 15 | 32.6 |
| 16 | 1.8 |
| 17 | 18.4 |
| 18 | 13.5 |
| 19 | 26.3 |
| 20 | 12.9 |
| 21 | 9.8 |
| 22 | 57.8 |
| 23 | 45.4 |
| 24 | 17.6 |
| 25 | 51.4 |
| 26 | 18.9 |
| 27 | 17.6 |
| 28 | 4.4 |
| 29 | 39.1 |
| 30 | 48.6 |
| 31 | 24.1 |
| 32 | 49.2 |
| 33 | 120.9 |
| 34 | 32.3 |
| 35 | 43.2 |
| 36 | 23.4 |
| 37 | 89.6 |
| 38 | 24.3 |
| 39 | 10.3 |
| 40 | 22.4 |
| 41 | 19.6 |
| 42 | 20.3 |
| 43 | 6.0 |
| 44 | 14.7 |
| 45 | 32.2 |
| 46 | 6.5 |
| 47 | 139.6 |
| 48 | 222.0 |
| 49 | 21.2 |
| 50 | 24.7 |
| 51 | 21.6 |
| 52 | 25.7 |
| 53 | 7.7 |
| 54 | 19.0 |
| 55 | 13.1 |
| 56 | 25.7 |
| 57 | 19.9 |
| 58 | 89.8 |
| 59 | 38.7 |
| 60 | 9.3 |
| 61 | 35.2 |
| 62 | 24.7 |
| 63 | 13.2 |
| 64 | 67.5 |
| 65 | 40.8 |
| 66 | 9.7 |
| 67 | 19.1 |
| 68 | 8.8 |
| 69 | 10.6 |
| 70 | 6.7 |
| 71 | 19.3 |
| 72 | 3.5 |
| 73 | 4.1 |
| 74 | 5.7 |
| 75 | 12.5 |
| 76 | 2.5 |
| 77 | 7.4 |
| 78 | 46.9 |
| 79 | 20.7 |
| 80 | 31.0 |
| 81 | 40.1 |
| 82 | 86.5 |
| 83 | 259.3 |
| 84 | 65.6 |
| 85 | 162.0 |
| 86 | 113.7 |
| 87 | 7.9 |
| 88 | 2.3 |
| 89 | 5.7 |
| 90 | 29.1 |
| 91 | 89.9 |
| 92 | 34.6 |
| 93 | 9.7 |
| 94 | 22.2 |
| 95 | 4.6 |
| 96 | 1.9 |
| 97 | 15.3 |
| 98 | 41.6 |
| 99 | 14.0 |
| 100 | 6.4 |
| 101 | 110.4 |
| 102 | 19.6 |
| 103 | 12.0 |
| 104 | 30.4 |
| 105 | 23.3 |
| 106 | 7.4 |
| 107 | 44.6 |
| 108 | 1.9 |
| 109 | 5.6 |
| 110 | 2.8 |
| 111 | 8.1 |
| 112 | 3.8 |
| 113 | 2.6 |
| 114 | 1.1 |
| 115 | 9.4 |
| 116 | 6.7 |
| 117 | 2.0 |
| 118 | 1.0 |
| 119 | 2.2 |
| 120 | 3.7 |
| 121 | 24.6 |
| 122 | 8.2 |
| 123 | 12.1 |
| 124 | 8.6 |
| 125 | 3.2 |
| 126 | 21.3 |
| 127 | 26.8 |
| 128 | 0.9 |
| 129 | 7.6 |
| 130 | 5.8 |
| 131 | 9.2 |
| 132 | 2.1 |
| 133 | 11.2 |
| 134 | 21.7 |
| 135 | 10.2 |
| 136 | 7.5 |
| 137 | 2.4 |
| 138 | 3.5 |
| 139 | 3.6 |
| 140 | 1.0 |
| 141 | 8.7 |
| 142 | 4.3 |
| 143 | 1.2 |
| 144 | 0.7 |
| 145 | 17.2 |
| 146 | 11.4 |
| 147 | 1.3 |
| 148 | 0.9 |
| 149 | 8.9 |
| 150 | 8.9 |
| 151 | 4.5 |
| 152 | 3.7 |
| 153 | 1.1 |
| 154 | 0.9 |
| 155 | 0.5 |
| 156 | 4.0 |
| 157 | 0.6 |
| 158 | 9.8 |
| 159 | 27.1 |
| 160 | 3.5 |
| 161 | 1.2 |
| 162 | 2.4 |
| 163 | 10.9 |
| 164 | 4.0 |
| 165 | 16.7 |
| 166 | 7.1 |
| 167 | 1.1 |
| 168 | 4.1 |
| 169 | 2.2 |
| 170 | 10.6 |
| 171 | 5.0 |
| 172 | 1.5 |
| 173 | 8.6 |
| 174 | 7.8 |
| 175 | 4.7 |
| 176 | 58.1 |
| 177 | 3.9 |
| 178 | 3.4 |
| 179 | 23.2 |
| 180 | 11.3 |
| 181 | 176.6 |
| 182 | 1.3 |
| 183 | 4.5 |
| 184 | 8.0 |
| 185 | 16.2 |
| 186 | 3.0 |
| 187 | 20.3 |
| 188 | 0.6 |
| 189 | 0.4 |
| 190 | 420.2 |
| 191 | 20.7 |
| 192 | 2.0 |
| 193 | 3.8 |
| 194 | 11.4 |
| 195 | 1.0 |
| 196 | 1.8 |
| 197 | 1.1 |
| 198 | 1.2 |
| 199 | 5.4 |
| 200 | 2.1 |
| 201 | 7.9 |
| 202 | 16.2 |
| 203 | 1.6 |
| 204 | 39.8 |
| 205 | 0.7 |
| 206 | 0.4 |
| 207 | 2.2 |
| 208 | 2.2 |
| 209 | 0.8 |
| 210 | 1.9 |
| 211 | 4.9 |
| 212 | 71.0 |
| 213 | 1.0 |
| 214 | 0.5 |
| 215 | 7.9 |
| 216 | 4.8 |
| 217 | 2.2 |
| 218 | 6.1 |
| 219 | 23.3 |
| 220 | 1.1 |
| 221 | 4.2 |
| 222 | 3.5 |
| 223 | 2.0 |
| 224 | 3.9 |
| 225 | 18.3 |
| 226 | 25.4 |
| 227 | 16.4 |
| 228 | 4.4 |
| 229 | 1.9 |
| 230 | 8.3 |
| 231 | 1.1 |
| 232 | 0.5 |
| 233 | 0.4 |
| 234 | 0.8 |
| 235 | 1.5 |
| 236 | 1.8 |
| 237 | 4.7 |
| 238 | 0.9 |
| 239 | 2.5 |
| 240 | 4.1 |
| 241 | 3.4 |
| 242 | 3.2 |
| 243 | 1.1 |
| 244 | 2.8 |
| 245 | 3.6 |
| 246 | 2.8 |
| 247 | 1.8 |
| 248 | 0.5 |
| 249 | 2.2 |
| 250 | 86.1 |
| 251 | 2.0 |
| 252 | 2.0 |
| 253 | 1.9 |
| 254 | 2.2 |
| 255 | 2.2 |
| 256 | 3.6 |
| 257 | 1.3 |
| 258 | 28.3 |
| 259 | 1.0 |
| 260 | 6.3 |
| 261 | 1.2 |
| 262 | 1.3 |
| 263 | 2.5 |
| 264 | 0.9 |
| 265 | 3.0 |
| 266 | 1.3 |
| 267 | 2.7 |
| 268 | 0.2 |
| 269 | 98.2 |
| 270 | 0.3 |
| 271 | 3.7 |
| 272 | 7.9 |
| 273 | 7.5 |
| 274 | 58.5 |
| 275 | 7.3 |
| 276 | 0.6 |
| 277 | 5.4 |
| 278 | 1.7 |
| 279 | 5.1 |
| 280 | 4.1 |
| 281 | 1.4 |
| 282 | 23.2 |
| 283 | 0.6 |
| 284 | 12.9 |
| 285 | 2.8 |
| 286 | 4.1 |
| 287 | 2.9 |
| 288 | 4.6 |
| 289 | 27.1 |
| 290 | 19.7 |
| 291 | 4.4 |
| 292 | 12.1 |
| 293 | 12.8 |
| 294 | 20.8 |
| 295 | 14.7 |
| 296 | 16.2 |
| 297 | 78.0 |
| 298 | 1.2 |
| 299 | 6.8 |
| 300 | 20.5 |
| 301 | 23.9 |
| 302 | 7.0 |
| 303 | 2.5 |
| 304 | 93.9 |
| 305 | 1.0 |
| 306 | 20.0 |
| 307 | 99.2 |
| 308 | 3.8 |
| 309 | 5.6 |
| 310 | 1.1 |
| 311 | 2.1 |
| 312 | 7.4 |
| 313 | 18.1 |
| 314 | 22.2 |
| 315 | 11.7 |
| 316 | 3.8 |
| 317 | 8.2 |
| 318 | 4.3 |
| 319 | 82.1 |
| 320 | 11.7 |
| 321 | 7.0 |
| 322 | 25.9 |
| 323 | 3.6 |
| 324 | 13.4 |
| 325 | 167.0 |
| 326 | 29.7 |
| 327 | 31.9 |
| 328 | 2.9 |
| 329 | 35.4 |
| 330 | 11.3 |
| 331 | 101.8 |
| 332 | 11.8 |
| 333 | 12.6 |
| 334 | 13.0 |
| 335 | 7.6 |
| 336 | 52.2 |
| 337 | 8.2 |
| 338 | 35.0 |
| 339 | 13.5 |
| 340 | 1.0 |
| 341 | 6.8 |
| 342 | 1.0 |
| 343 | 3.3 |
| 344 | 9.5 |
| 345 | 43.2 |
| 346 | 5.2 |
| 347 | 1.1 |
| 348 | 1.2 |
| 349 | 3.0 |
| 350 | 4.8 |
| 351 | 2.1 |
| 352 | 8.9 |
| 353 | 5.7 |
| 354 | 2.2 |
| 355 | 11.7 |
| 356 | 4.6 |
| 357 | 0.6 |
| 358 | 0.8 |
| 359 | 8.0 |
| 360 | 0.9 |
| 361 | 1.8 |
| 362 | 4.3 |
| 363 | 3.2 |
| 364 | 10.1 |
| 365 | 8.0 |
| 366 | 10.0 |
| 367 | 4.7 |
| 368 | 7.9 |
| 369 | 4.5 |
| 370 | 8.1 |
| 371 | 8.8 |
| 372 | 1.6 |
| 373 | 4.6 |
| 374 | 1.6 |
| 375 | 6.7 |
| 376 | 33.2 |
| 377 | 41.6 |
| 378 | 7.0 |
| 379 | 4.5 |
| 380 | 26.3 |
| 381 | 0.7 |
| 382 | 13.4 |
| 383 | 1.6 |
| 384 | 25.6 |
| 385 | 2.2 |
| 386 | 0.3 |
| 387 | 0.2 |
| 388 | 0.3 |
| 389 | 0.8 |
| 390 | 3.5 |
| 391 | 5.7 |
| 392 | 3.1 |
| 393 | 12.7 |
| 394 | 4.9 |
| 395 | 4.4 |
| 396 | 2.4 |
| 397 | 2.4 |
| 398 | 10.6 |
| 399 | 11.2 |
| 400 | 2.3 |
| 401 | 3.2 |
| 402 | 4.7 |
| 403 | 0.7 |
| 404 | 0.4 |
| 405 | 0.2 |
| 406 | 0.3 |
| 407 | 0.3 |
| 408 | 1.2 |
| 409 | 0.2 |
| 410 | 0.3 |
| 411 | 3.7 |
| 412 | 1.0 |
| 413 | 0.8 |
| 414 | 38.0 |
| 415 | 50.6 |
| 416 | 2.7 |
| 417 | 1.9 |
| 418 | 2.0 |
| 419 | 0.5 |
| 420 | 0.4 |
| 421 | 1.7 |
| 422 | 7.1 |
| 423 | 6.1 |
| 424 | 2.0 |
| 425 | 1.2 |
| 426 | 65.0 |
| 427 | 12.0 |
| 428 | 5.2 |
| 429 | 1.6 |
| 430 | 1.8 |
| 431 | 2.6 |
| 432 | 1.6 |
| 433 | 1.1 |
| 434 | 1.2 |
| 435 | 1.1 |
| 436 | 0.6 |
| 437 | 1.8 |
| 438 | 0.6 |
| 439 | 1.9 |
| 440 | 2.1 |
| 441 | 5.7 |
| 442 | 3.4 |
| 443 | 3.1 |
| 444 | 5.8 |
| 445 | 5.2 |
| 446 | 2.7 |
| 447 | 4.8 |
| 448 | 19.0 |
| 449 | 15.2 |
| 450 | 29.8 |
| 451 | 8.0 |
| 452 | 5.1 |
| 453 | 4.9 |
| 454 | 0.7 |
| 455 | 5.2 |
| 456 | 17.1 |
| 457 | 5.8 |
| 458 | 4.6 |
| 459 | 3.0 |
| 460 | 1.3 |
| 461 | 0.8 |
| 462 | 1.3 |
| 463 | 1.1 |
| 464 | 0.2 |
| 465 | 0.6 |
| 466 | 0.9 |
| 467 | 0.7 |
| 468 | 5.2 |
| 469 | 4.0 |
| 470 | 3.1 |
| 471 | 2.5 |
| 472 | 4.1 |
| 473 | 1.0 |
| 474 | 2.1 |
| 475 | 116.5 |
| 476 | 7.6 |
| 477 | 1.1 |
| 478 | 9.5 |
| 479 | 66.1 |
| 480 | 3.2 |
| 481 | 0.6 |
| 482 | 0.8 |
| 483 | 8.5 |
| 484 | 2.6 |
| 485 | 0.6 |
| 486 | 0.4 |
| 487 | 0.2 |
| 488 | 5.3 |
| 489 | 1.6 |
| 490 | 2.8 |
| 491 | 2.2 |
| 492 | 3.2 |
| 493 | 25.0 |
| 494 | 12.0 |
| 495 | 8.8 |
| 496 | 2.8 |
| 497 | 0.2 |
| 498 | 0.7 |
| 499 | 0.3 |
| 500 | 0.8 |
| 501 | 1.4 |
| 502 | 2.1 |
| 503 | 7.3 |
| 504 | 30.6 |
| 505 | 1.5 |
| 506 | 5.7 |
| 507 | 0.6 |
| 508 | 0.8 |
| 509 | 8.1 |
| 510 | 0.1 |
| 511 | 3.8 |
| 512 | 1.6 |
| 513 | 11.2 |
| 514 | 0.6 |
| 515 | 3.9 |
| 516 | 1.4 |
| 517 | 3.2 |
| 518 | 0.8 |
| 519 | 0.3 |
| 520 | 1.7 |
| 521 | 3.3 |
| 522 | 0.7 |
| 523 | 0.6 |
| 524 | 0.3 |
| 525 | 1.3 |
| 526 | 0.5 |
| 527 | 0.6 |
| 528 | 1.2 |
| 529 | 4.1 |
| 530 | 10.9 |
| 531 | 5.0 |
| 532 | 8.1 |
| 533 | 4.7 |
| 534 | 0.8 |
| 535 | 0.7 |
| 536 | 1.2 |
| 537 | 4.0 |
| 538 | 0.4 |
| 539 | 12.0 |

### Example 62—MOLM-13 Uridine Rescue Assay

Several compounds were also tested in a Uridine rescue assay. In the Uridine rescue assay, using the procedure in Example 1 above, an additional plate of MOLM-13 cell cultures was prepared, to which 100 μM of Uridine was added in step 3 of the process. The IC₅₀ (nM) results of the assay are shown in Table 2, below.

TABLE 2

| Cpd | IC₅₀ |
|---|---|
| 72 | 18750 |
| 108 | 8270 |
| 157 | 14890 |
| 197 | 17480 |
| 213 | 7600 |
| 220 | 6700 |
| 231 | 19270 |
| 233 | 12410 |
| 248 | 8550 |
| 253 | 6560 |
| 254 | 6910 |
| 255 | 15620 |
| 264 | 20880 |
| 265 | 10740 |
| 271 | 22690 |
| 272 | 7680 |
| 273 | 29670 |
| 278 | 4810 |
| 282 | 11520 |
| 284 | 12160 |
| 288 | 11420 |
| 293 | 10560 |
| 294 | 12430 |
| 305 | 11580 |
| 310 | 14450 |
| 311 | 8470 |
| 316 | 10680 |
| 320 | 9810 |
| 324 | 9720 |
| 328 | 10540 |
| 330 | 11530 |
| 340 | 15240 |
| 343 | 19750 |
| 347 | 2770 |
| 348 | 2000 |
| 350 | 14890 |
| 352 | 6410 |
| 355 | 5660 |
| 356 | 6960 |
| 357 | 9300 |
| 358 | 12280 |
| 359 | 5670 |
| 361 | 22.090 |
| 362 | 6.810 |
| 363 | 10.29 |
| 369 | 5.020 |
| 372 | 16.390 |
| 374 | 8.840 |
| 381 | 4.290 |
| 387 | 7.660 |
| 388 | 6.640 |
| 398 | 0.720 |
| 405 | 5.880 |
| 406 | 8.350 |
| 407 | 14.700 |
| 408 | 10.990 |
| 409 | 12.560 |
| 410 | 14.200 |
| 418 | 14.880 |
| 419 | 5.640 |
| 420 | 3.120 |
| 460 | 1.160 |
| 462 | 13.60 |

The results of the uridine rescue assay above for all compounds tested in the presence of uridine rescued cellular proliferation in the MOLM assay compared to the absence of uridine (see Table 1). These results indicate that compounds described herein function as DHODH inhibitors in the de novo pyrimidine synthesis pathway.

Although certain aspects have been described in detail above, those having ordinary skill in the art will clearly understand that many modifications are possible without departing from the teachings thereof, and are intended to be encompassed within the claims as described herein.

1. A compound of Formula (I): 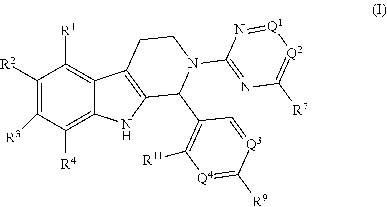
or a form thereof, wherein:
R¹ is hydrogen, deuterium, amino, nitro or fluoro;
R² is halo, C₁₋₄alkyl, C₂₋₄alkenyl, nitro, C₁₋₄alkoxy, (C₁₋₄alkyl)₂amino, pyrrolyl, pyrazolyl, 3-methyl-pyrazolyl, 4-methyl-pyrazolyl, 2H-1,2,3-triazol-2-yl, or 4-methyl-2H-1,2,3-triazolyl-2-yl;
R³ is hydrogen, deuterium, amino or fluoro;
R⁴ is hydrogen, amino or hydroxy;
Q¹ is CR⁵ or N;
Q² is CR⁶ or N;
wherein Q¹ and Q² are not simultaneously N;
R⁵ and R⁷ are independently selected from hydrogen, deuterium, halo, cyano, C₁₋₄alkyl, halo-C₁₋₄alkyl, hydroxy-C₁₋₄alkyl, C₁₋₄alkyl-thio, C₁₋₄alkoxy, amino, (C₁₋₄alkyl)amino, (C₁₋₄alkyl)₂amino, C₁₋₄alkoxycarbonyl, phenyl and heterocyclyl, wherein heterocyclyl is a saturated or partially unsaturated 3-7 membered monocyclic ring system radical having one, two, or three heteroatom ring members independently selected from N, O, or S; and, wherein phenyl and heterocyclyl are optionally substituted with one, two, or three R¹² substituents;
R⁶ is hydrogen, deuterium, halo, C₁₋₆alkyl, halo-C₁₋₄alkyl, phenyl or morpholinyl;
wherein, when Q² is N, then R⁵ and R⁶ are other than halo; and,
wherein R³, R⁶ and R⁷ are not all simultaneously hydrogen;
Q¹ is CR⁸ or N;
R⁸ is hydrogen or deuterium;
R⁹ is hydrogen, deuterium, halo, cyano, hydroxy, C₁₋₄alkyl, halo-C₁₋₄alkyl, C₁₋₄alkenyl, C₁₋₄alkylcarbonyl, amino, (C₁₋₄alkyl)amino, (C₁₋₄alkyl)₂amino, (C₁₋₄alkyl)₂amino(C₁₋₄alkyl)amino, C₁₋₄alkylthio, C₁₋₄alkyl-sulfonyl, amino-sulfonyl, C₁₋₄alkoxy, C₁₋₄alkoxyC₁₋₄alkoxy, (C₁₋₄alkoxy)carbonyl, (C₁₋₄alkoxy)carbonylC₁₋₄alkyl, hydroxyC₁₋₄alkyl, hydroxyC₂₋₄alkenyl, hydroxyC₁₋₄alkylamino, hydroxyC₁₋₄alkoxyC₁₋₄alkyl, carboxyl-C₁₋₄alkyl, carboxyl_C₁₋₄alkyl_amino, carboxyl_C₁₋₄alkoxy_C₁₋₄alkylamino, C₃₋₆cycloalkyl, heteroaryl or heterocyclyl, wherein heteroaryl is an aromatic 5-6 membered monocyclic ring having one, two, or three heteroatom ring members independently selected from N, O, or S; wherein heterocyclyl is a saturated or partially unsaturated 3-7 membered monocyclic, 6-10 membered bicyclic, 7-8 membered bicyclic, or 13-16 membered polycyclic ring system having one, two, or three heteroatom ring members independently selected from N, O, or S; and, wherein each heteroaryl or heterocyclyl is optionally substituted with one, two, or three R¹² substituents;
Q⁴ is CR¹⁰ or N;
R¹⁰ is hydrogen, deuterium, fluoro or hydroxy;
R¹¹ is hydrogen, deuterium, fluoro or hydroxy; and,
wherein R⁸, R⁹, R¹⁰ and R¹¹ are not simultaneously hydrogen; and,
R¹² is halo, hydroxy, C₁₋₆alkyl or C₃₋₆cycloalkyl; and,
wherein the form of the compound is selected from the group consisting of salt, hydrate, solvate, racemate, enantiomer, diastereomer, stereoisomer, and tautomer thereof.

2. The compound of claim 1, wherein R² is pyrrolyl, pyrazolyl, 3-methyl-pyrazolyl, 4-methyl-pyrazolyl, 2H-1,2,3-triazol-2-yl, or 4-methyl-2H-1,2,3-triazolyl-2-yl.

3. The compound of claim 1, wherein R² is halo, C₁₋₄alkyl, C₂₋₄alkenyl, nitro, C₁₋₄alkoxy or (C₁₋₄alkyl)₂amino.

4. The compound of claim 1, wherein R² is chloro, fluoro, bromo, methyl, ethyl, ethenyl, nitro, methoxy, dimethyl-amino, 1H-pyrrol-1-yl, 1H-pyrazol-1-yl, 3-methyl-1H-pyrazol-1-yl, 4-methyl-1H-pyrazol-1-yl, 2H-1,2,3-triazol-2-yl, or 4-methyl-2H-1,2,3-triazol-2-yl.

5. The compound of claim 1, wherein R⁵ and R⁷ are independently selected from hydrogen, halo, cyano, C₁₋₄alkyl, halo-C₁₋₄alkyl, hydroxy-C₁₋₄alkyl, C₁₋₄alkyl-thio, C₁₋₄alkoxy, amino, (C₁₋₄alkyl)₂amino, C₁₋₄alkoxycarbonyl, phenyl, or heterocyclyl,
wherein heterocyclyl is selected from morpholinyl, piperazinyl and azetidinyl, and
wherein heterocyclyl is optionally substituted with one, two, or three R¹² substituents.

6. The compound of claim 1, wherein R⁵ and R⁷ are independently selected from hydrogen, fluoro, chloro, bromo, cyano, methyl, ethyl, isopropyl, fluoro-methyl, difluoro-methyl, trifluoromethyl, trichloromethyl, hydroxy-methyl, methyl-thio, methoxy, butoxy, amino, N,N-dimethyl-amino, N-methyl-N-butyl-amino, N,N-dibutyl-amino, methoxy-carbonyl, butoxy-carbonyl, phenyl, morpholinyl, 3,3-difluoro-azetidin-1-yl, 1-methylpiperazin-4-yl, or phenyl.

7. The compound of claim 1, wherein R⁶ is hydrogen, chloro, fluoro, bromo, methyl, trifluoromethyl, or phenyl.

8. The compound of claim 1, wherein R⁹ is hydrogen, deuterium, halo, cyano, C₁₋₄ alkyl, halo-C₁₋₄alkyl, C₂₋₄alkenyl, C₁₋₄alkylcarbonyl, amino, (C₁₋₄alkyl)amino, (C₁₋₄alkyl)₂amino, (C₁₋₄alkyl)₂amino(C₁₋₄alkyl)amino, C₁₋₄alkylthio, C₁₋₄alkyl-sulfonyl, amino_sulfonyl, C₁₋₄alkoxy, C₁₋₄alkoxy-C₁₋₄alkoxy, (C₁₋₄alkoxy)carbonyl, hydroxyC₁₋₄alkyl, hydroxyC₂₋₄alkenyl, hydroxyC₁₋₄alkylamino, (C₁₋₄alkoxy)carbonylC₁₋₄alkyl, hydroxyC₁₋₄alkoxyC₁₋₄alkyl, carboxyl-C₁₋₄alkyl, carboxyl-C₁₋₄alkylamino, carboxyl-C₁₋₄alkoxy-C₁₋₄alkylamino, C₃₋₆cycloalkyl, heteroaryl or heterocyclyl, wherein heteroaryl is an unsaturated 5-6 membered monocyclic ring having one, two, or three heteroatom ring members independently selected from N, O, or S, wherein heterocyclyl is a saturated or partially unsaturated 7-8 membered bicyclic ring system having one, two, or three heteroatom ring members independently selected from N, O, or S, and, wherein each heterocyclyl or heteroaryl is optionally substituted with one, two, or three R¹² substituents.

9. The compound of claim 1, wherein R⁹ is hydrogen, fluoro, chloro, bromo, cyano, methyl, isopropyl, trifluoromethyl, ethenyl, methylcarbonyl, amino, N-methylamino, N,N-dimethylamino, N—[(N,N-dimethylamino)ethyl]amino, methyl-thio, methyl-sulfonyl, amino-sulfonyl, methoxy, methoxy-ethoxy, methoxycarbonyl, hydroxymethyl, hydroxyethyl, hydroxypropyl, hydroxybutyl, 1-hydroxy-but-3-en-4-yl, N-(hydroxyethyl)amino, methoxy-carbonyl-ethyl, hydroxy-ethoxy-methyl, carboxy-ethyl, carboxy-propyl, N-(carboxy-ethyl)amino, N-(carboxy-propyl)amino, N-(carboxy-methoxy-ethyl)amino, cyclopropyl, 1H-imidazolyl, 1H-pyrazolyl, 1H-1,2,4-triazolyl, morpholinyl, piperazinyl, 4-methyl-piperazinyl, 3,3-dimethyl-piperazinyl, azetidinyl, 2-oxa-5-azabicyclo[2.2.1]heptanyl, 3-oxa-8-azabicyclo[3.2.1]octanyl, (1S,4S)-2-oxa-5-azabicyclo[2.2.1]heptanyl, (1R,4R)-2-oxa-5-azabicyclo[2.2.1]heptanyl, (3R,5S)-3,5-dimethylpiperazinyl, (3R)-3-methylpiperazinyl, (3S)-3-methylpiperazinyl, 4,7-diazaspiro[2.5]octanyl, or 2-oxa-6-azaspiro[3.3]heptanyl.

10. A compound selected from the group consisting of:
6-chloro-2-(5-chloropyrimidin-2-yl)-1-(4-fluorophenyl)-2,3,4,9-tetrahydro-1H-pyrido[3,4-b]indole;
6-chloro-1-(4-fluorophenyl)-2-(5-methylpyrimidin-2-yl)-2,3,4,9-tetrahydro-1H-pyrido[3,4-b]indole;
6-chloro-1-(4-fluorophenyl)-2-(5-fluoropyrimidin-2-yl)-2,3,4,9-tetrahydro-1H-pyrido[3,4-b]indole;
6-chloro-2-(5-chloropyrimidin-2-yl)-7-fluoro-1-(4-fluorophenyl)-2,3,4,9-tetrahydro-1H-pyrido[3,4-b]indole;
6-chloro-7-fluoro-1-(4-fluorophenyl)-2-(5-fluoropyrimidin-2-yl)-2,3,4,9-tetrahydro-1H-pyrido[3,4-b]indole;
6-chloro-7-fluoro-2-(5-fluoropyrimidin-2-yl)-1-(4-methylphenyl)-2,3,4,9-tetrahydro-1H-pyrido[3,4-b]indole;
6-chloro-2-(4,6-dichloropyrimidin-2-yl)-1-(4-methylphenyl)-2,3,4,9-tetrahydro-1H-pyrido[3,4-b]indole;
6-chloro-7-fluoro-2-(5-fluoropyrimidin-2-yl)-1-(6-methoxypyridin-3-yl)-2,3,4,9-tetrahydro-1H-pyrido[3,4-b]indole;
6-chloro-7-fluoro-1-(4-fluorophenyl)-2-(4-methylpyrimidin-2-yl)-2,3,4,9-tetrahydro-1H-pyrido[3,4-b]indole;
6-chloro-2-(4-chloropyrimidin-2-yl)-1-(4-methylphenyl)-2,3,4,9-tetrahydro-1H-pyrido[3,4-b]indole;
6-chloro-1-(4-chlorophenyl)-7-fluoro-2-(5-fluoropyrimidin-2-yl)-2,3,4,9-tetrahydro-1H-pyrido[3,4-b]indole;
6-chloro-1-(2,4-difluorophenyl)-7-fluoro-2-(5-fluoropyrimidin-2-yl)-2,3,4,9-tetrahydro-1H-pyrido[3,4-b]indole
6-chloro-2-(4-chloropyrimidin-2-yl)-1-(4-fluorophenyl)-2,3,4,9-tetrahydro-1H-pyrido[3,4-b]indole;
5-[6-chloro-7-fluoro-2-(5-fluoropyrimidin-2-yl)-2,3,4,9-tetrahydro-1H-pyrido[3,4-b]indol-1-yl]-N,N-dimethylpyrimidin-2-amine;
6-chloro-2-(4-chloropyrimidin-2-yl)-7-fluoro-1-(4-fluorophenyl)-2,3,4,9-tetrahydro-1H-pyrido[3,4-b]indole;
6-chloro-2-(4,6-dichloropyrimidin-2-yl)-7-fluoro-1-(4-methylphenyl)-2,3,4,9-tetrahydro-1H-pyrido[3,4-b]indole;
6-chloro-2-[6-chloro-7-fluoro-1-(4-methylphenyl)-1,3,4,9-tetrahydro-2H-pyrido[3,4-b]indol-2-yl]-N,N-dimethylpyrimidin-4-amine;
4-[6-chloro-2-(4-chloropyrimidin-2-yl)-7-fluoro-2,3,4,9-tetrahydro-1H-pyrido[3,4-b]indol-1-yl]benzonitrile;
6-chloro-2-(4,6-dimethylpyrimidin-2-yl)-7-fluoro-1-(4-fluorophenyl)-2,3,4,9-tetrahydro-1H-pyrido[3,4-b]indole;
6-chloro-5-fluoro-2-(5-fluoropyrimidin-2-yl)-1-(4-methylphenyl)-2,3,4,9-tetrahydro-1H-pyrido[3,4-b]indole;
6-chloro-2-(5-chloropyrimidin-2-yl)-5-fluoro-1-(4-methylphenyl)-2,3,4,9-tetrahydro-1H-pyrido[3,4-b]indole;
6-chloro-2-(4-ethylpyrimidin-2-yl)-7-fluoro-1-(4-fluorophenyl)-2,3,4,9-tetrahydro-1H-pyrido[3,4-b]indole;
6-chloro-7-fluoro-1-(4-fluorophenyl)-2-[4-(propan-2-yl)pyrimidin-2-yl]-2,3,4,9-tetrahydro-1H-pyrido[3,4-b]indole;
6-chloro-7-fluoro-1-(4-fluorophenyl)-2-[4-(trifluoromethyl)pyrimidin-2-yl]-2,3,4,9-tetrahydro-1H-pyrido[3,4-b]indole;
2-[6-chloro-7-fluoro-1-(4-fluorophenyl)-1,3,4,9-tetrahydro-2H-pyrido[3,4-b]indol-2-yl]pyrimidine-4-carbonitrile;
6-chloro-1-(4-chlorophenyl)-5-fluoro-2-(5-fluoropyrimidin-2-yl)-2,3,4,9-tetrahydro-1H-pyrido[3,4-b]indole;
6-chloro-1-(4-chlorophenyl)-2-(5-chloropyrimidin-2-yl)-5-fluoro-2,3,4,9-tetrahydro-1H-pyrido[3,4-b]indole;
6-chloro-2-(4-chloropyrimidin-2-yl)-5-fluoro-1-(4-methylphenyl)-2,3,4,9-tetrahydro-1H-pyrido[3,4-b]indole;
6-chloro-1-(4-chlorophenyl)-2-(5-chloropyrimidin-2-yl)-7-fluoro-2,3,4,9-tetrahydro-1H-pyrido[3,4-b]indole;
6-chloro-7-fluoro-1-(2-fluorophenyl)-2-(5-fluoropyrimidin-2-yl)-2,3,4,9-tetrahydro-1H-pyrido[3,4-b]indole;
6-chloro-1-(4-cyclopropylphenyl)-7-fluoro-2-(5-fluoropyrimidin-2-yl)-2,3,4,9-tetrahydro-1H-pyrido[3,4-b]indole;
4-[6-chloro-7-fluoro-2-(5-fluoropyrimidin-2-yl)-2,3,4,9-tetrahydro-1H-pyrido[3,4-b]indol-1-yl]benzonitrile;
4-[6-chloro-8-fluoro-2-(5-fluoropyrimidin-2-yl)-2,3,4,9-tetrahydro-1H-pyrido[3,4-b]indol-1-yl]benzonitrile;
5-[6-chloro-7-fluoro-2-(5-fluoropyrimidin-2-yl)-2,3,4,9-tetrahydro-1H-pyrido[3,4-b]indol-1-yl]-N,N-dimethylpyridin-2-amine;
6-chloro-7-fluoro-2-(5-fluoropyrimidin-2-yl)-1-[4-(trifluoromethyl)phenyl]-2,3,4,9-tetrahydro-1H-pyrido[3,4-b]indole;
6-chloro-1-(2,3-difluorophenyl)-7-fluoro-2-(5-fluoropyrimidin-2-yl)-2,3,4,9-tetrahydro-1H-pyrido[3,4-b]indole;
6-chloro-1-(3,4-difluorophenyl)-7-fluoro-2-(5-fluoropyrimidin-2-yl)-2,3,4,9-tetrahydro-1H-pyrido[3,4-b]indole;
6-chloro-1-(4-chlorophenyl)-7-fluoro-2-(4-methylpyrimidin-2-yl)-2,3,4,9-tetrahydro-1H-pyrido[3,4-b]indole;
6-chloro-1-(4-chlorophenyl)-2-(4-chloropyrimidin-2-yl)-7-fluoro-2,3,4,9-tetrahydro-1H-pyrido[3,4-b]indole;
2-[6-chloro-1-(4-chlorophenyl)-7-fluoro-1,3,4,9-tetrahydro-2H-pyrido[3,4-b]indol-2-yl]pyrimidine-4-carbonitrile;
6-chloro-2-(5-chloropyrimidin-2-yl)-7-fluoro-1-(4-methylphenyl)-2,3,4,9-tetrahydro-1H-pyrido[3,4-b]indole;
6-chloro-7-fluoro-1-(4-methylphenyl)-2-(4-methylpyrimidin-2-yl)-2,3,4,9-tetrahydro-1H-pyrido[3,4-b]indole;
6-chloro-2-(4-chloropyrimidin-2-yl)-7-fluoro-1-(4-methylphenyl)-2,3,4,9-tetrahydro-1H-pyrido[3,4-b]indole;
2-[6-chloro-7-fluoro-1-(4-methylphenyl)-1,3,4,9-tetrahydro-2H-pyrido[3,4-b]indol-2-yl]pyrimidine-4-carbonitrile;
6-chloro-1-(4-cyclopropylphenyl)-7-fluoro-2-(pyrimidin-2-yl)-2,3,4,9-tetrahydro-1H-pyrido[3,4-b]indole;
6-chloro-2-(4-chloro-6-methylpyrimidin-2-yl)-7-fluoro-1-(4-fluorophenyl)-2,3,4,9-tetrahydro-1H-pyrido[3,4-b]indole;
6-chloro-7-fluoro-1-(6-fluoropyridin-3-yl)-2-(5-fluoropyrimidin-2-yl)-2,3,4,9-tetrahydro-1H-pyrido[3,4-b]indole;
5-[6-chloro-7-fluoro-2-(5-fluoropyrimidin-2-yl)-2,3,4,9-tetrahydro-1H-pyrido[3,4-b]indol-1-yl]pyridin-2-ol;
6-chloro-2-(5-chloropyrimidin-2-yl)-5-fluoro-1-(4-fluorophenyl)-2,3,4,9-tetrahydro-1H-pyrido[3,4-b]indole;
6-chloro-7-fluoro-2-(5-fluoropyrimidin-2-yl)-1-[4-(propan-2-yl)phenyl]-2,3,4,9-tetrahydro-1H-pyrido[3,4-b]indole;
6-chloro-5-fluoro-1-(4-fluorophenyl)-2-(5-fluoropyrimidin-2-yl)-2,3,4,9-tetrahydro-1H-pyrido[3,4-b]indole;
4-[6-chloro-2-(5-chloropyrimidin-2-yl)-5-fluoro-2,3,4,9-tetrahydro-1H-pyrido[3,4-b]indol-1-yl]benzonitrile;
6-chloro-2-(4-chloropyrimidin-2-yl)-7-fluoro-1-[4-(propan-2-yl)phenyl]-2,3,4,9-tetrahydro-1H-pyrido[3,4-b]indole;
6-chloro-7-fluoro-2-(4-methylpyrimidin-2-yl)-1-[4-(propan-2-yl)phenyl]-2,3,4,9-tetrahydro-1H-pyrido[3,4-b]indole;
2-{6-chloro-7-fluoro-1-[4-(propan-2-yl)phenyl]-1,3,4,9-tetrahydro-2H-pyrido[3,4-b]indol-2-yl}pyrimidine-4-carbonitrile;
4-[6-chloro-5-fluoro-2-(5-fluoropyrimidin-2-yl)-2,3,4,9-tetrahydro-1H-pyrido[3,4-b]indol-1-yl]benzonitrile;
6-chloro-2-(5-chloropyrimidin-2-yl)-7-fluoro-1-[4-(propan-2-yl)phenyl]-2,3,4,9-tetrahydro-1H-pyrido[3,4-b]indole;
6-chloro-7-fluoro-1-(3-fluorophenyl)-2-(5-fluoropyrimidin-2-yl)-2,3,4,9-tetrahydro-1H-pyrido[3,4-b]indole;
4-[6-chloro-2-(5-chloropyrimidin-2-yl)-7-fluoro-2,3,4,9-tetrahydro-1H-pyrido[3,4-b]indol-1-yl]benzonitrile;
6-chloro-5-fluoro-1-(4-fluorophenyl)-2-[4-(trifluoromethyl)pyrimidin-2-yl]-2,3,4,9-tetrahydro-1H-pyrido[3,4-b]indole;
4-[6-chloro-7-fluoro-2-(4-methylpyrimidin-2-yl)-2,3,4,9-tetrahydro-1H-pyrido[3,4-b]indol-1-yl]benzonitrile;
6-chloro-2-(5-chloropyrimidin-2-yl)-5-fluoro-1-[4-(trifluoromethyl)phenyl]-2,3,4,9-tetrahydro-1H-pyrido[3,4-b]indole;
6-chloro-1-(2,3-difluorophenyl)-7-fluoro-2-(4-methylpyrimidin-2-yl)-2,3,4,9-tetrahydro-1H-pyrido[3,4-b]indole;
2-[6-chloro-1-(4-cyanophenyl)-7-fluoro-1,3,4,9-tetrahydro-2H-pyrido[3,4-b]indol-2-yl]pyrimidine-4-carbonitrile;
{4-[6-chloro-2-(5-fluoropyrimidin-2-yl)-2,3,4,9-tetrahydro-1H-pyrido[3,4-b]indol-1-yl]phenyl}methanol;
6-chloro-2-(4,6-dimethylpyrimidin-2-yl)-7-fluoro-1-(4-methylphenyl)-2,3,4,9-tetrahydro-1H-pyrido[3,4-b]indole;
6-chloro-5-fluoro-2-(5-fluoropyrimidin-2-yl)-1-[4-(trifluoromethyl)phenyl]-2,3,4,9-tetrahydro-1H-pyrido[3,4-b]indole;
2-[6-chloro-1-(2,3-difluorophenyl)-7-fluoro-1,3,4,9-tetrahydro-2H-pyrido[3,4-b]indol-2-yl]pyrimidine-4-carbonitrile;
4-[2-(4-bromopyrimidin-2-yl)-6-chloro-7-fluoro-2,3,4,9-tetrahydro-1H-pyrido[3,4-b]indol-1-yl]benzonitrile;
6-chloro-1-(4-chlorophenyl)-5-fluoro-2-(5-fluoro-4-methylpyrimidin-2-yl)-2,3,4,9-tetrahydro-1H-pyrido[3,4-b]indole;
6-chloro-1-(4-ethenylphenyl)-2-(5-fluoropyrimidin-2-yl)-2,3,4,9-tetrahydro-1H-pyrido[3,4-b]indole;
6-chloro-7-fluoro-1-(4-methylphenyl)-2-[4-(trifluoromethyl)pyrimidin-2-yl]-2,3,4,9-tetrahydro-1H-pyrido[3,4-b]indole;
6-chloro-1-(4-chlorophenyl)-5-fluoro-2-[4-(trifluoromethyl)pyrimidin-2-yl]-2,3,4,9-tetrahydro-1H-pyrido[3,4-b]indole;
6-chloro-5-fluoro-1-[4-(trifluoromethyl)phenyl]-2-[4-(trifluoromethyl)pyrimidin-2-yl]-2,3,4,9-tetrahydro-1H-pyrido[3,4-b]indole;
6-chloro-7-fluoro-1-[4-(trifluoromethyl)phenyl]-2-[4-(trifluoromethyl)pyrimidin-2-yl]-2,3,4,9-tetrahydro-1H-pyrido[3,4-b]indole;
6-chloro-5-fluoro-1-(4-methylphenyl)-2-[4-(trifluoromethyl)pyrimidin-2-yl]-2,3,4,9-tetrahydro-1H-pyrido[3,4-b]indole;
6-chloro-2-(4-chloropyrimidin-2-yl)-1-(2,3-difluorophenyl)-7-fluoro-2,3,4,9-tetrahydro-1H-pyrido[3,4-b]indole;
{4-[6-chloro-7-fluoro-2-(5-fluoropyrimidin-2-yl)-2,3,4,9-tetrahydro-1H-pyrido[3,4-b]indol-1-yl]phenyl}methanol;
6-chloro-1-(4-ethenylphenyl)-7-fluoro-2-(5-fluoropyrimidin-2-yl)-2,3,4,9-tetrahydro-1H-pyrido[3,4-b]indole;
6-chloro-2-(5-chloropyrimidin-2-yl)-7-fluoro-1-[4-(trifluoromethyl)phenyl]-2,3,4,9-tetrahydro-1H-pyrido[3,4-b]indole;
6-chloro-7-fluoro-2-(4-methylpyrimidin-2-yl)-1-[4-(trifluoromethyl)phenyl]-2,3,4,9-tetrahydro-1H-pyrido[3,4-b]indole;
5,6-difluoro-2-(5-fluoropyrimidin-2-yl)-1-(4-methylphenyl)-2,3,4,9-tetrahydro-1H-pyrido[3,4-b]indole;
5,6-difluoro-1-(4-fluorophenyl)-2-(5-fluoropyrimidin-2-yl)-2,3,4,9-tetrahydro-1H-pyrido[3,4-b]indole;
2-(5-chloropyrimidin-2-yl)-5,6-difluoro-1-(4-methylphenyl)-2,3,4,9-tetrahydro-1H-pyrido[3,4-b]indole;
2-(5-chloropyrimidin-2-yl)-5,6-difluoro-1-(4-fluorophenyl)-2,3,4,9-tetrahydro-1H-pyrido[3,4-b]indole;
2-{4-[6-chloro-7-fluoro-2-(5-fluoropyrimidin-2-yl)-2,3,4,9-tetrahydro-1H-pyrido[3,4-b]indol-1-yl]phenyl}ethan-1-ol;
methyl 2-[6-chloro-1-(4-chlorophenyl)-5-fluoro-1,3,4,9-tetrahydro-2H-pyrido[3,4-b]indol-2-yl]pyrimidine-4-carboxylate;
4-{6-chloro-7-fluoro-2-[4-methyl-6-(trifluoromethyl)pyrimidin-2-yl]-2,3,4,9-tetrahydro-1H-pyrido[3,4-b]indol-1-yl}benzonitrile;
4-{6-chloro-5-fluoro-2-[4-(trifluoromethyl)pyrimidin-2-yl]-2,3,4,9-tetrahydro-1H-pyrido[3,4-b]indol-1-yl}benzonitrile;
6-chloro-5-fluoro-1-(4-fluorophenyl)-2-(4-methylpyrimidin-2-yl)-2,3,4,9-tetrahydro-1H-pyrido[3,4-b]indole;
6-chloro-7-fluoro-2-(5-fluoropyrimidin-2-yl)-1-(2-methylpyrimidin-5-yl)-2,3,4,9-tetrahydro-1H-pyrido[3,4-b]indole;
6-chloro-7-fluoro-1-(2-methylpyrimidin-5-yl)-2-[4-(trifluoromethyl)pyrimidin-2-yl]-2,3,4,9-tetrahydro-1H-pyrido[3,4-b]indole;
4-[6-chloro-2-(4,6-dimethylpyrimidin-2-yl)-7-fluoro-2,3,4,9-tetrahydro-1H-pyrido[3,4-b]indol-1-yl]benzonitrile;
5-[6-chloro-7-fluoro-2-(4-methylpyrimidin-2-yl)-2,3,4,9-tetrahydro-1H-pyrido[3,4-b]indol-1-yl]-N,N-dimethylpyrimidin-2-amine;
5-{6-chloro-7-fluoro-2-[4-(trifluoromethyl)pyrimidin-2-yl]-2,3,4,9-tetrahydro-1H-pyrido[3,4-b]indol-1-yl}-N,N-dimethylpyrimidin-2-amine;
6-chloro-7-fluoro-1-(4-methylphenyl)-2-[4-methyl-6-(trifluoromethyl)pyrimidin-2-yl]-2,3,4,9-tetrahydro-1H-pyrido[3,4-b]indole;
6-chloro-7-fluoro-1-(4-methylphenyl)-2-[5-(trifluoromethyl)pyrimidin-2-yl]-2,3,4,9-tetrahydro-1H-pyrido[3,4-b]indole;
6-chloro-7-fluoro-1-(4-fluorophenyl)-2-[5-(trifluoromethyl)pyrimidin-2-yl]-2,3,4,9-tetrahydro-1H-pyrido[3,4-b]indole;
6-chloro-7-fluoro-1-[4-(propan-2-yl)phenyl]-2-[5-(trifluoromethyl)pyrimidin-2-yl]-2,3,4,9-tetrahydro-1H-pyrido[3,4-b]indole;
4-{6-chloro-7-fluoro-2-[4-(trifluoromethyl)pyrimidin-2-yl]-2,3,4,9-tetrahydro-1H-pyrido[3,4-b]indol-1-yl}benzonitrile;
6-chloro-2-(5-chloropyrimidin-2-yl)-7-fluoro-1-(2-methylpyrimidin-5-yl)-2,3,4,9-tetrahydro-1H-pyrido[3,4-b]indole;
5-[6-chloro-2-(5-chloropyrimidin-2-yl)-7-fluoro-2,3,4,9-tetrahydro-1H-pyrido[3,4-b]indol-1-yl]-N,N-dimethylpyrimidin-2-amine;
6-chloro-7-fluoro-1-[4-(methanesulfonyl)phenyl]-2-[4-(trifluoromethyl)pyrimidin-2-yl]-2,3,4,9-tetrahydro-1H-pyrido[3,4-b]indole;
methyl 2-[6-chloro-1-(4-cyanophenyl)-5-fluoro-1,3,4,9-tetrahydro-2H-pyrido[3,4-b]indol-2-yl]pyrimidine-4-carboxylate;
6-chloro-7-fluoro-2-(5-fluoro-4-methylpyrimidin-2-yl)-1-(4-fluorophenyl)-2,3,4,9-tetrahydro-1H-pyrido[3,4-b]indole;
6-chloro-7-fluoro-2-(5-fluoro-4,6-dimethylpyrimidin-2-yl)-1-(4-methylphenyl)-2,3,4,9-tetrahydro-1H-pyrido[3,4-b]indole;
6-chloro-7-fluoro-2-(5-fluoropyrimidin-2-yl)-1-[4-(methanesulfonyl)phenyl]-2,3,4,9-tetrahydro-1H-pyrido[3,4-b]indole;
6-chloro-1-(2,3-difluorophenyl)-7-fluoro-2-[4-(trifluoromethyl)pyrimidin-2-yl]-2,3,4,9-tetrahydro-1H-pyrido[3,4-b]indole;
6-chloro-5-fluoro-2-(5-fluoro-4-methylpyrimidin-2-yl)-1-(4-methylphenyl)-2,3,4,9-tetrahydro-1H-pyrido[3,4-b]indole;
2-[6-chloro-5-fluoro-1-(4-methylphenyl)-1,3,4,9-tetrahydro-2H-pyrido[3,4-b]indol-2-yl]-6-methylpyrimidine-4-carbonitrile;
2-[6-chloro-5-fluoro-1-(4-fluorophenyl)-1,3,4,9-tetrahydro-2H-pyrido[3,4-b]indol-2-yl]-6-methylpyrimidine-4-carbonitrile;
6-chloro-1-(2,3-difluorophenyl)-7-fluoro-2-(5-fluoro-4,6-dimethylpyrimidin-2-yl)-2,3,4,9-tetrahydro-1H-pyrido[3,4-b]indole;
6-chloro-1-(2,3-difluorophenyl)-5-fluoro-2-(5-fluoro-4-methylpyrimidin-2-yl)-2,3,4,9-tetrahydro-1H-pyrido[3,4-b]indole;
6-chloro-1-(2,3-difluoro-4-methylphenyl)-5-fluoro-2-(5-fluoro-4-methylpyrimidin-2-yl)-2,3,4,9-tetrahydro-1H-pyrido[3,4-b]indole
4-{6-chloro-7-fluoro-2-[4-(trifluoromethyl)pyrimidin-2-yl]-2,3,4,9-tetrahydro-1H-pyrido[3,4-b]indol-1-yl}benzene-1-sulfonamide;
6-chloro-5-fluoro-2-(5-fluoro-4,6-dimethylpyrimidin-2-yl)-1-(4-methylphenyl)-2,3,4,9-tetrahydro-1H-pyrido[3,4-b]indole;
6-chloro-1-(2,3-difluorophenyl)-5-fluoro-2-(5-fluoro-4,6-dimethylpyrimidin-2-yl)-2,3,4,9-tetrahydro-1H-pyrido[3,4-b]indole;
6-chloro-1-(2,3-difluoro-4-methylphenyl)-5-fluoro-2-(5-fluoro-4,6-dimethylpyrimidin-2-yl)-2,3,4,9-tetrahydro-1H-pyrido[3,4-b]indole;
6-chloro-1-(2,3-difluorophenyl)-2-[4-(trifluoromethyl)pyrimidin-2-yl]-2,3,4,9-tetrahydro-1H-pyrido[3,4-b]indole;
6-chloro-1-(2,3-difluoro-4-methoxyphenyl)-2-[4-(trifluoromethyl)pyrimidin-2-yl]-2,3,4,9-tetrahydro-1H-pyrido[3,4-b]indole;
2-{6-chloro-7-fluoro-1-[4-(trifluoromethyl)phenyl]-1,3,4,9-tetrahydro-2H-pyrido[3,4-b]indol-2-yl}pyrimidine-4-carbonitrile;
5-{6-chloro-7-fluoro-2-[4-(trifluoromethyl)pyrimidin-2-yl]-2,3,4,9-tetrahydro-1H-pyrido[3,4-b]indol-1-yl}-N,N-dimethylpyridin-2-amine;
6-chloro-7-fluoro-2-(5-fluoro-4-methylpyrimidin-2-yl)-1-(4-methylphenyl)-2,3,4,9-tetrahydro-1H-pyrido[3,4-b]indole;
6-chloro-1-(2,3-difluorophenyl)-7-fluoro-2-(5-fluoro-4-methylpyrimidin-2-yl)-2,3,4,9-tetrahydro-1H-pyrido[3,4-b]indole;
6-chloro-1-(2,3-difluoro-4-methylphenyl)-7-fluoro-2-(5-fluoro-4-methylpyrimidin-2-yl)-2,3,4,9-tetrahydro-1H-pyrido[3,4-b]indole;
5-[6-chloro-7-fluoro-2-(4-methylpyrimidin-2-yl)-2,3,4,9-tetrahydro-1H-pyrido[3,4-b]indol-1-yl]-N,N-dimethylpyridin-2-amine;
4-[6-chloro-7-fluoro-2-(5-fluoropyrimidin-2-yl)-2,3,4,9-tetrahydro-1H-pyrido[3,4-b]indol-1-yl]benzene-1-sulfonamide;
6-chloro-1-(2,3-difluoro-4-methylphenyl)-2-[4-(trifluoromethyl)pyrimidin-2-yl]-2,3,4,9-tetrahydro-1H-pyrido[3,4-b]indole;
6-chloro-1-(2,3-difluorophenyl)-2-(4,6-dimethylpyrimidin-2-yl)-7-fluoro-2,3,4,9-tetrahydro-1H-pyrido[3,4-b]indole;
6-chloro-2-(5-chloropyrimidin-2-yl)-1-(2,3-difluoro-4-methylphenyl)-7-fluoro-2,3,4,9-tetrahydro-1H-pyrido[3,4-b]indole;
6-chloro-2-(5-chloropyrimidin-2-yl)-1-(2,3-difluorophenyl)-7-fluoro-2,3,4,9-tetrahydro-1H-pyrido[3,4-b]indole;
6-chloro-1-(2,3-difluoro-4-methylphenyl)-2-(4,6-dimethylpyrimidin-2-yl)-7-fluoro-2,3,4,9-tetrahydro-1H-pyrido[3,4-b]indole;
6-chloro-1-(2,3-difluorophenyl)-7-fluoro-2-[5-(trifluoromethyl)pyrimidin-2-yl]-2,3,4,9-tetrahydro-1H-pyrido[3,4-b]indole;
6-chloro-7-fluoro-1-(4-methylphenyl)-2-(5-methylpyrimidin-2-yl)-2,3,4,9-tetrahydro-1H-pyrido[3,4-b]indole;
6-chloro-5-fluoro-1-(4-methylphenyl)-2-(5-methylpyrimidin-2-yl)-2,3,4,9-tetrahydro-1H-pyrido[3,4-b]indole;
6-chloro-1-(2,3-difluoro-4-methylphenyl)-7-fluoro-2-(5-methylpyrimidin-2-yl)-2,3,4,9-tetrahydro-1H-pyrido[3,4-b]indole;
6-chloro-1-(2,3-difluoro-4-methylphenyl)-5-fluoro-2-(5-methylpyrimidin-2-yl)-2,3,4,9-tetrahydro-1H-pyrido[3,4-b]indole;
6-chloro-1-(2,3-difluoro-4-methylphenyl)-7-fluoro-2-(5-fluoropyrimidin-2-yl)-2,3,4,9-tetrahydro-1H-pyrido[3,4-b]indole;
6-chloro-1-(2,3-difluoro-4-methylphenyl)-7-fluoro-2-(4-methylpyrimidin-2-yl)-2,3,4,9-tetrahydro-1H-pyrido[3,4-b]indole;
6-chloro-1-(2,3-difluoro-4-methylphenyl)-7-fluoro-2-[4-(trifluoromethyl)pyrimidin-2-yl]-2,3,4,9-tetrahydro-1H-pyrido[3,4-b]indole;
2-[6-chloro-1-(2,3-difluoro-4-methylphenyl)-7-fluoro-1,3,4,9-tetrahydro-2H-pyrido[3,4-b]indol-2-yl]pyrimidine-4-carbonitrile;
6-chloro-1-(2,3-difluoro-4-methylphenyl)-7-fluoro-2-[5-(trifluoromethyl)pyrimidin-2-yl]-2,3,4,9-tetrahydro-1H-pyrido[3,4-b]indole;
6-chloro-1-(2,3-difluoro-4-methylphenyl)-7-fluoro-2-(5-fluoro-4,6-dimethylpyrimidin-2-yl)-2,3,4,9-tetrahydro-1H-pyrido[3,4-b]indole;
6-chloro-1-(4-chloro-2,3-difluorophenyl)-2-[4-(trifluoromethyl)pyrimidin-2-yl]-2,3,4,9-tetrahydro-1H-pyrido[3,4-b]indole;
6-chloro-7-fluoro-1-(5-fluoropyridin-3-yl)-2-[4-(trifluoromethyl)pyrimidin-2-yl]-2,3,4,9-tetrahydro-1H-pyrido[3,4-b]indole;
6-chloro-5-fluoro-1-(5-fluoropyridin-3-yl)-2-[4-(trifluoromethyl)pyrimidin-2-yl]-2,3,4,9-tetrahydro-1H-pyrido[3,4-b]indole;
6-chloro-1-(2,3-difluorophenyl)-7-fluoro-2-[4-methyl-6-(trifluoromethyl)pyrimidin-2-yl]-2,3,4,9-tetrahydro-1H-pyrido[3,4-b]indole;
6-chloro-1-(2,3-difluoro-4-methylphenyl)-7-fluoro-2-[4-methyl-6-(trifluoromethyl)pyrimidin-2-yl]-2,3,4,9-tetrahydro-1H-pyrido[3,4-b]indole;
6-chloro-5-fluoro-1-[4-(methanesulfonyl)phenyl]-2-[4-(trifluoromethyl)pyrimidin-2-yl]-2,3,4,9-tetrahydro-1H-pyrido[3,4-b]indole;
4-{6-chloro-5-fluoro-2-[4-(trifluoromethyl)pyrimidin-2-yl]-2,3,4,9-tetrahydro-1H-pyrido[3,4-b]indol-1-yl}benzene-1-sulfonamide;
2-[6-chloro-7-fluoro-1-(4-methylphenyl)-1,3,4,9-tetrahydro-2H-pyrido[3,4-b]indol-2-yl]-6-methylpyrimidine-4-carbonitrile;
2-[6-chloro-1-(2,3-difluorophenyl)-7-fluoro-1,3,4,9-tetrahydro-2H-pyrido[3,4-b]indol-2-yl]-6-methylpyrimidine-4-carbonitrile;
2-[6-chloro-1-(2,3-difluoro-4-methylphenyl)-7-fluoro-1,3,4,9-tetrahydro-2H-pyrido[3,4-b]indol-2-yl]-6-methylpyrimidine-4-carbonitrile;
2-[4,6-bis(trifluoromethyl)pyrimidin-2-yl]-6-chloro-7-fluoro-1-(4-methylphenyl)-2,3,4,9-tetrahydro-1H-pyrido[3,4-b]indole;
2-[4,6-bis(trifluoromethyl)pyrimidin-2-yl]-6-chloro-1-(2,3-difluoro-4-methylphenyl)-7-fluoro-2,3,4,9-tetrahydro-1H-pyrido[3,4-b]indole;
3-(4-{6-chloro-7-fluoro-2-[4-(trifluoromethyl)pyrimidin-2-yl]-2,3,4,9-tetrahydro-1H-pyrido[3,4-b]indol-1-yl}phenyl)propan-1-ol;
2-[4,6-bis(trifluoromethyl)pyrimidin-2-yl]-6-chloro-1-(2,3-difluorophenyl)-7-fluoro-2,3,4,9-tetrahydro-1H-pyrido[3,4-b]indole;
5-{6-chloro-7-fluoro-2-[4-(trifluoromethyl)pyrimidin-2-yl]-2,3,4,9-tetrahydro-1H-pyrido[3,4-b]indol-1-yl}-3-fluoro-N,N-dimethylpyridin-2-amine;
3-(4-{6-chloro-7-fluoro-2-(5-fluoropyrimidin-2-yl)-2,3,4,9-tetrahydro-1H-pyrido[3,4-b]indol-1-yl]phenyl}propan-1-ol;
5-{6-chloro-5-fluoro-2-[4-(trifluoromethyl)pyrimidin-2-yl]-2,3,4,9-tetrahydro-1H-pyrido[3,4-b]indol-1-yl)-3-fluoro-N,N-dimethylpyridin-2-amine;
(1S)-6-chloro-1-(4-methylphenyl)-2-[4-(trifluoromethyl)pyrimidin-2-yl]-2,3,4,9-tetrahydro-1H-pyrido[3,4-b]indole;
2-[6-chloro-1-(2,3-difluoro-4-methylphenyl)-7-fluoro-1,3,4,9-tetrahydro-2H-pyrido[3,4-b]indol-2-yl]-5-fluoropyrimidine-4-carbonitrile;
6-chloro-7-fluoro-1-(4-methylphenyl)-2-(4,5,6-trimethylpyrimidin-2-yl)-2,3,4,9-tetrahydro-1H-pyrido[3,4-b]indole;
methyl 3-(4-(6-chloro-7-fluoro-2-[4-(trifluoromethyl)pyrimidin-2-yl]-2,3,4,9-tetrahydro-1H-pyrido[3,4-b]indol-1-yl}phenyl)propanoate;
methyl 3-{4-[6-chloro-7-fluoro-2-(5-fluoropyrimidin-2-yl)-2,3,4,9-tetrahydro-1H-pyrido[3,4-b]indol-1-yl]phenyl}propanoate;
(4-(6-chloro-7-fluoro-2-[4-(trifluoromethyl)pyrimidin-2-yl]-2,3,4,9-tetrahydro-1H-pyrido[3,4-b]indol-1-ylphenyl)methanol;
(3E)-4-(4-{6-chloro-7-fluoro-2-[4-(trifluoromethyl)pyrimidin-2-yl]-2,3,4,9-tetrahydro-1H-pyrido[3,4-b]indol-1-yl}phenyl)but-3-en-1-ol;
4-(4-{6-chloro-7-fluoro-2-[4-(trifluoromethyl)pyrimidin-2-yl]-2,3,4,9-tetrahydro-1H-pyrido[3,4-b]indol-1-yl}phenyl)butan-1-ol;
6-chloro-1-(2,3-difluoro-4-methylphenyl)-7-fluoro-2-(4,5,6-trimethylpyrimidin-2-yl)-2,3,4,9-tetrahydro-1H-pyrido[3,4-b]indole;
4-{6-chloro-7-fluoro-2-[4-(trifluoromethyl)pyrimidin-2-yl]-2,3,4,9-tetrahydro-1H-pyrido[3,4-b]indol-1-yl}-N,N-dimethylaniline;
6-chloro-1-(2,3-difluorophenyl)-7-fluoro-2-(4,5,6-trimethylpyrimidin-2-yl)-2,3,4,9-tetrahydro-1H-pyrido[3,4-b]indole;
1-(4-bromo-2,3-difluorophenyl)-6-chloro-7-fluoro-2-[4-(trifluoromethyl)pyrimidin-2-yl]-2,3,4,9-tetrahydro-1H-pyrido[3,4-b]indole;
4-{6-chloro-7-fluoro-2-[4-(trifluoromethyl)pyrimidin-2-yl]-2,3,4,9-tetrahydro-1H-pyrido[3,4-b]indol-1-yl}-2,3-difluoro-N,N-dimethylaniline;
5-fluoro-6-methyl-1-(4-methylphenyl)-2-[4-(trifluoromethyl)pyrimidin-2-yl]-2,3,4,9-tetrahydro-1H-pyrido[3,4-b]indole;
2-[6-chloro-7-fluoro-1-(4-methylphenyl)-1,3,4,9-tetrahydro-2H-pyrido[3,4-b]indol-2-yl]-5-fluoropyrimidine-4-carbonitrile;
2-[(1R)-6-chloro-1-(2,3-difluoro-4-methylphenyl)-1,3,4,9-tetrahydro-2H-pyrido[3,4-b]indol-2-yl]pyrimidine-4-carbonitrile;
5-{6-chloro-5-fluoro-2-[4-(trifluoromethyl)pyrimidin-2-yl]-2,3,4,9-tetrahydro-1H-pyrido[3,4-b]indol-1-yl}-N,N-dimethylpyridin-2-amine;
5-{6-chloro-5-fluoro-2-[4-(trifluoromethyl)pyrimidin-2-yl]-2,3,4,9-tetrahydro-1H-pyrido[3,4-b]indol-1-yl}-N,N-dimethylpyrimidin-2-amine;
(1R)-6-chloro-1-(2,3-difluoro-4-methylphenyl)-2-(5-fluoropyrimidin-2-yl)-2,3,4,9-tetrahydro-1H-pyrido[3,4-b]indole;
(1R)-6-chloro-1-(2,3-difluoro-4-methylphenyl)-2-[4-(trifluoromethyl)pyrimidin-2-yl]-2,3,4,9-tetrahydro-1H-pyrido[3,4-b]indole;
(1R)-6-chloro-1-(4-chlorophenyl)-5-fluoro-2-[4-(trifluoromethyl)pyrimidin-2-yl]-2,3,4,9-tetrahydro-1H-pyrido[3,4-b]indole;
(1S)-6-chloro-1-(4-chlorophenyl)-5-fluoro-2-[4-(trifluoromethyl)pyrimidin-2-yl]-2,3,4,9-tetrahydro-1H-pyrido[3,4-b]indole;
2-[6-chloro-1-(2,3-difluorophenyl)-7-fluoro-1,3,4,9-tetrahydro-2H-pyrido[3,4-b]indol-2-yl]-5-fluoropyrimidine-4-carbonitrile;
6-chloro-1-(2,6-difluoropyridin-3-yl)-7-fluoro-2-[4-(trifluoromethyl)pyrimidin-2-yl]-2,3,4,9-tetrahydro-1H-pyrido[3,4-b]indole;
(1R)-6-chloro-1-(2,3-difluoro-4-methylphenyl)-2-(4-methylpyrimidin-2-yl)-2,3,4,9-tetrahydro-1H-pyrido[3,4-b]indole;
6-chloro-1-(2,6-difluoropyridin-3-yl)-5-fluoro-2-[4-(trifluoromethyl)pyrimidin-2-yl]-2,3,4,9-tetrahydro-1H-pyrido[3,4-b]indole;
(1R)-6-chloro-1-(2,3-difluoro-4-methylphenyl)-2-(5-fluoro-4-methylpyrimidin-2-yl)-2,3,4,9-tetrahydro-1H-pyrido[3,4-b]indole;
4-{2-[4,6-bis(trifluoromethyl)pyrimidin-2-yl]-6-chloro-7-fluoro-2,3,4,9-tetrahydro-1H-pyrido[3,4-b]indol-1-yl}benzonitrile;
(1S)-6-chloro-1-(2,3-difluoro-4-methylphenyl)-5-fluoro-2-(5-fluoro-4,6-dimethylpyrimidin-2-yl)-2,3,4,9-tetrahydro-1H-pyrido[3,4-b]indole;
(1R)-6-chloro-1-(2,3-difluoro-4-methylphenyl)-5-fluoro-2-(5-fluoro-4,6-dimethylpyrimidin-2-yl)-2,3,4,9-tetrahydro-1H-pyrido[3,4-b]indole;
6-chloro-5-fluoro-1-(pyrimidin-5-yl)-2-[4-(trifluoromethyl)pyrimidin-2-yl]-2,3,4,9-tetrahydro-1H-pyrido[3,4-b]indole;
1-(2,3-difluoro-4-methylphenyl)-5-fluoro-6-methyl-2-[4-(trifluoromethyl)pyrimidin-2-yl]-2,3,4,9-tetrahydro-1H-pyrido[3,4-b]indole;
6-chloro-5-fluoro-1-[2-(1H-imidazol-1-yl)pyrimidin-5-yl]-2-[4-(trifluoromethyl)pyrimidin-2-yl]-2,3,4,9-tetrahydro-1H-pyrido[3,4-b]indole;
5-[6-chloro-5-fluoro-2-(5-fluoropyrimidin-2-yl)-2,3,4,9-tetrahydro-1H-pyrido[3,4-b]indol-1-yl]-N,N-dimethylpyridin-2-amine;
6-chloro-2-(5-chloropyrimidin-2-yl)-1-(2,3-difluoro-4-methylphenyl)-5-fluoro-2,3,4,9-tetrahydro-1H-pyrido[3,4-b]indole;
6-chloro-1-(2,3-difluoro-4-methylphenyl)-5-fluoro-2-(4-methylpyrimidin-2-yl)-2,3,4,9-tetrahydro-1H-pyrido[3,4-b]indole;
6-chloro-1-(2,3-difluoro-4-methylphenyl)-5-fluoro-2-[4-(trifluoromethyl)pyrimidin-2-yl]-2,3,4,9-tetrahydro-1H-pyrido[3,4-b]indole;
2-[6-chloro-1-(2,3-difluoro-4-methylphenyl)-5-fluoro-1,3,4,9-tetrahydro-2H-pyrido[3,4-b]indol-2-yl]pyrimidine-4-carbonitrile;
5-(6-chloro-5-fluoro-2-[4-(trifluoromethyl)pyrimidin-2-yl]-2,3,4,9-tetrahydro-1H-pyrido[3,4-b]indol-1-yl)-N-methylpyrimidin-2-amine;
6-chloro-5-fluoro-1-[2-(morpholin-4-yl)pyrimidin-5-yl]-2-[4-(trifluoromethyl)pyrimidin-2-yl]-2,3,4,9-tetrahydro-1H-pyrido[3,4-b]indole;
5-[6-chloro-5-fluoro-2-(5-fluoropyrimidin-2-yl)-2,3,4,9-tetrahydro-1H-pyrido[3,4-b]indol-1-yl]-N,N-dimethylpyrimidin-2-amine;
6-chloro-5-fluoro-1-(pyridin-3-yl)-2-[4-(trifluoromethyl)pyrimidin-2-yl]-2,3,4,9-tetrahydro-1H-pyrido[3,4-b]indole;
6-chloro-1-(2,3-difluoro-4-methylphenyl)-2-(4-methylpyrimidin-2-yl)-2,3,4,9-tetrahydro-1H-pyrido[3,4-b]indole;
(1R)-6-chloro-1-(2,3-difluoro-4-methylphenyl)-2-[4-methyl-6-(trifluoromethyl)pyrimidin-2-yl]-2,3,4,9-tetrahydro-1H-pyrido[3,4-b]indole;
6-chloro-1-(2,3-difluoro-4-methylphenyl)-2-(4,6-dimethylpyrimidin-2-yl)-5-fluoro-2,3,4,9-tetrahydro-1H-pyrido[3,4-b]indole;
6-chloro-1-(2,3-difluoro-4-methylphenyl)-5-fluoro-2-[4-methyl-6-(trifluoromethyl)pyrimidin-2-yl]-2,3,4,9-tetrahydro-1H-pyrido[3,4-b]indole;
6-chloro-1-(4-methylphenyl)-2-[4-(trifluoromethyl)pyrimidin-2-yl]-2,3,4,9-tetrahydro-1H-pyrido[3,4-b]indol-8-ol;
5-(6-chloro-7-fluoro-2-[4-(trifluoromethyl)pyrimidin-2-yl]-2,3,4,9-tetrahydro-1H-pyrido[3,4-b]indol-1-yl)-6-fluoro-N,N-dimethylpyridin-2-amine;
2-[4,6-bis(trifluoromethyl)pyrimidin-2-yl]-6-chloro-1-(2,3-difluoro-4-methylphenyl)-5-fluoro-2,3,4,9-tetrahydro-1H-pyrido[3,4-b]indole;
5-{6-chloro-5-fluoro-2-[4-(trifluoromethyl)pyrimidin-2-yl]-2,3,4,9-tetrahydro-1H-pyrido[3,4-b]indol-1-yl}-6-fluoro-N,N-dimethylpyridin-2-amine;
6-chloro-5-fluoro-2-(5-fluoropyrimidin-2-yl)-1-[2-(morpholin-4-yl)pyrimidin-5-yl]-2,3,4,9-tetrahydro-1H-pyrido[3,4-b]indole;
5-[6-chloro-5-fluoro-2-(5-fluoropyrimidin-2-yl)-2,3,4,9-tetrahydro-1H-pyrido[3,4-b]indol-1-yl]pyrimidin-2-amine;
6-chloro-1-(2,3-difluoro-4-methylphenyl)-2-[4-methyl-6-(trifluoromethyl)pyrimidin-2-yl]-2,3,4,9-tetrahydro-1H-pyrido[3,4-b]indole;
2-[6-chloro-1-(2,3-difluoro-4-methylphenyl)-5-fluoro-1,3,4,9-tetrahydro-2H-pyrido[3,4-b]indol-2-yl]-5-fluoropyrimidine-4-carbonitrile;
2-[6-chloro-7-fluoro-1-(4-methylphenyl)-1,3,4,9-tetrahydro-2H-pyrido[3,4-b]indol-2-yl]pyrimidine-4,6-dicarbonitrile;
2-[6-chloro-1-(2,3-difluoro-4-methylphenyl)-7-fluoro-1,3,4,9-tetrahydro-2H-pyrido[3,4-b]indol-2-yl]pyrimidine-4,6-dicarbonitrile;
2-[6-chloro-5-fluoro-1-(4-methylphenyl)-1,3,4,9-tetrahydro-2H-pyrido[3,4-b]indol-2-yl]-5-fluoropyrimidine-4-carbonitrile;
2-chloro-5-(6-chloro-2-[4-(trifluoromethyl)pyrimidin-2-yl]-2,3,4,9-tetrahydro-1H-pyrido[3,4-b]indol-1-yl)phenol;
2-chloro-5-[6-chloro-2-(5-fluoropyrimidin-2-yl)-2,3,4,9-tetrahydro-1H-pyrido[3,4-b]indol-1-yl]phenol;
6-chloro-1-(2,3-difluoro-4-methylphenyl)-2-[4-(trifluoromethyl)pyrimidin-2-yl]-2,3,4,9-tetrahydro-1H-pyrido[3,4-b]indol-8-ol;
5-chloro-2-[6-chloro-2-(5-fluoropyrimidin-2-yl)-2,3,4,9-tetrahydro-1H-pyrido[3,4-b]indol-1-yl]phenol;
5-chloro-2-(6-chloro-2-[4-(trifluoromethyl)pyrimidin-2-yl]-2,3,4,9-tetrahydro-1H-pyrido[3,4-b]indol-1-yl)phenol;
2-[(4-{6-chloro-7-fluoro-2-[4-(trifluoromethyl)pyrimidin-2-yl]-2,3,4,9-tetrahydro-1H-pyrido[3,4-b]indol-1-yl}phenyl)methoxy]ethan-1-ol;
2-(6-chloro-7-fluoro-1-{4-[(1E)-4-hydroxybut-1-en-1-yl]phenyl}-1,3,4,9-tetrahydro-2H-pyrido[3,4-b]indol-2-yl)pyrimidine-4-carbonitrile;
5-{6-chloro-5-fluoro-2-[4-(trifluoromethyl)pyrimidin-2-yl]-2,3,4,9-tetrahydro-1H-pyrido[3,4-b]indol-1-yl}pyrimidin-2-amine;
6-chloro-5-fluoro-1-(2-fluoropyridin-3-yl)-2-[4-(trifluoromethyl)pyrimidin-2-yl]-2,3,4,9-tetrahydro-1H-pyrido[3,4-b]indole;
2-[6-chloro-1-(2,3-difluorophenyl)-7-fluoro-1,3,4,9-tetrahydro-2H-pyrido[3,4-b]indol-2-yl]pyrimidine-4,6-dicarbonitrile;
2-[6-chloro-5-fluoro-1-(4-methylphenyl)-1,3,4,9-tetrahydro-2H-pyrido[3,4-b]indol-2-yl]pyrimidine-4,6-dicarbonitrile;
2-[6-chloro-1-(2,3-difluoro-4-methylphenyl)-5-fluoro-1,3,4,9-tetrahydro-2H-pyrido[3,4-b]indol-2-yl]pyrimidine-4,6-dicarbonitrile;
6-chloro-5-fluoro-1-[2-(1H-1,2,4-triazol-1-yl)pyrimidin-5-yl]-2-[4-(trifluoromethyl)pyrimidin-2-yl]-2,3,4,9-tetrahydro-1H-pyrido[3,4-b]indole;
6-chloro-1-(4-methylphenyl)-2-[4-(trifluoromethyl)pyrimidin-2-yl]-2,3,4,9-tetrahydro-1H-pyrido[3,4-b]indol-8-amine;
2-[6-chloro-1-(2,3-difluoro-4-methylphenyl)-5-fluoro-1,3,4,9-tetrahydro-2H-pyrido[3,4-b]indol-2-yl]-6-methylpyrimidine-4-carbonitrile;
(1S)-6-chloro-1-(2,3-difluoro-4-methylphenyl)-2-[4-(trifluoromethyl)pyrimidin-2-yl]-2,3,4,9-tetrahydro-1H-pyrido[3,4-b]indole;
(1S)-6-chloro-1-(2,3-difluoro-4-methylphenyl)-2-(4-methylpyrimidin-2-yl)-2,3,4,9-tetrahydro-1H-pyrido[3,4-b]indole;
6-chloro-1-(2,3-difluoro-4-methylphenyl)-2-(5-fluoro-4-methylpyrimidin-2-yl)-2,3,4,9-tetrahydro-1H-pyrido[3,4-b]indole;
2-(6-chloro-5-fluoro-1-{4-[(1E)-4-hydroxybut-1-en-1-yl]phenyl}-1,3,4,9-tetrahydro-2H-pyrido[3,4-b]indol-2-yl)pyrimidine-4-carbonitrile;
(3E)-4-{4-[6-chloro-5-fluoro-2-(4-methylpyrimidin-2-yl)-2,3,4,9-tetrahydro-1H-pyrido[3,4-b]indol-1-yl]phenyl}but-3-en-1-ol;
(1S)-6-chloro-1-(2,3-difluoro-4-methylphenyl)-2-(5-fluoropyrimidin-2-yl)-2,3,4,9-tetrahydro-1H-pyrido[3,4-b]indole;
(3E)-4-{4-[6-chloro-7-fluoro-2-(5-fluoro-4-methylpyrimidin-2-yl)-2,3,4,9-tetrahydro-1H-pyrido[3,4-b]indol-1-yl]phenyl}but-3-en-1-ol;
4-{4-[6-chloro-7-fluoro-2-(5-fluoro-4-methylpyrimidin-2-yl)-2,3,4,9-tetrahydro-1H-pyrido[3,4-b]indol-1-yl]phenyl}butan-1-ol;
(3E)-4-(4-[6-chloro-5-fluoro-2-(5-fluoro-4-methylpyrimidin-2-yl)-2,3,4,9-tetrahydro-1H-pyrido[3,4-b]indol-1-yl]phenyl)but-3-en-1-ol;
(3E)-4-{4-[6-chloro-2-(4,6-dimethylpyrimidin-2-yl)-5-fluoro-2,3,4,9-tetrahydro-1H-pyrido[3,4-b]indol-1-yl]phenyl}but-3-en-1-ol;
(3E)-4-(4-[6-chloro-5-fluoro-2-(5-fluoro-4,6-dimethylpyrimidin-2-yl)-2,3,4,9-tetrahydro-1H-pyrido[3,4-b]indol-1-yl]phenyl but-3-en-1-ol;
(3E)-4-{4-[6-chloro-2-(4,6-dimethylpyrimidin-2-yl)-7-fluoro-2,3,4,9-tetrahydro-1H-pyrido[3,4-b]indol-1-yl]phenyl}but-3-en-1-ol;
4-{4-[6-chloro-2-(4,6-dimethylpyrimidin-2-yl)-7-fluoro-2,3,4,9-tetrahydro-1H-pyrido[3,4-b]indol-1-yl]phenyl}butan-1-ol;
4-{4-[6-chloro-5-fluoro-2-(5-fluoro-4-methylpyrimidin-2-yl)-2,3,4,9-tetrahydro-1H-pyrido[3,4-b]indol-1-yl]phenyl}butan-1-ol;
5-bromo-6-chloro-1-(4-chlorophenyl)-2-[4-(trifluoromethyl)pyrimidin-2-yl]-2,3,4,9-tetrahydro-1H-pyrido[3,4-b]indole;
6-chloro-1-(2,3-difluoro-4-methylphenyl)-2-[4-(trifluoromethyl)pyrimidin-2-yl]-2,3,4,9-tetrahydro-1H-pyrido[3,4-b]indol-8-amine;
4-{4-[6-chloro-7-fluoro-2-(5-fluoro-4,6-dimethylpyrimidin-2-yl)-2,3,4,9-tetrahydro-1H-pyrido[3,4-b]indol-1-yl]phenyl}butan-1-ol;
6-chloro-1-(4-chlorophenyl)-2-[4-(trifluoromethyl)pyrimidin-2-yl]-2,3,4,9-tetrahydro-1H-pyrido[3,4-b]indol-7-ol;
6-chloro-1-[2-(morpholin-4-yl)pyrimidin-5-yl]-2-[4-(trifluoromethyl)pyrimidin-2-yl]-2,3,4,9-tetrahydro-1H-pyrido[3,4-b]indole;
2-{6-chloro-2-[4-(trifluoromethyl)pyrimidin-2-yl]-2,3,4,9-tetrahydro-1H-pyrido[3,4-b]indol-1-yl}-6-fluorophenol;
6-chloro-1-(2,3-difluoro-4-methylphenyl)-2-(4,6-dimethyl-1,3,5-triazin-2-yl)-2,3,4,9-tetrahydro-1H-pyrido[3,4-b]indole;
6-chloro-1-(2-fluoro-4-methylphenyl)-2-[4-(trifluoromethyl)pyrimidin-2-yl]-2,3,4,9-tetrahydro-1H-pyrido[3,4-b]indole;
4-(6-chloro-2-[4-(trifluoromethyl)pyrimidin-2-yl]-2,3,4,9-tetrahydro-1H-pyrido[3,4-b]indol-1-yl)-3-fluorobenzonitrile;
3-{6-chloro-2-[4-(trifluoromethyl)pyrimidin-2-yl]-2,3,4,9-tetrahydro-1H-pyrido[3,4-b]indol-1-yl}-2-fluorophenol;
2-{6-chloro-2-[4-(trifluoromethyl)pyrimidin-2-yl]-2,3,4,9-tetrahydro-1H-pyrido[3,4-b]indol-1-yl}-5-methylphenol;
5-{6-chloro-2-[4-(trifluoromethyl)pyrimidin-2-yl]-2,3,4,9-tetrahydro-1H-pyrido[3,4-b]indol-1-yl}-2-methylphenol;
6-chloro-1-(2-fluoro-3-methylphenyl)-2-[4-(trifluoromethyl)pyrimidin-2-yl]-2,3,4,9-tetrahydro-1H-pyrido[3,4-b]indole;
6-chloro-1-[4-(1H-imidazol-1-yl)phenyl]-2-[4-(trifluoromethyl)pyrimidin-2-yl]-2,3,4,9-tetrahydro-1H-pyrido[3,4-b]indole;
6-chloro-1-[4-(1H-pyrazol-1-yl)phenyl]-2-[4-(trifluoromethyl)pyrimidin-2-yl]-2,3,4,9-tetrahydro-1H-pyrido[3,4-b]indole;
6-chloro-1-[4-(morpholin-4-yl)phenyl]-2-[4-(trifluoromethyl)pyrimidin-2-yl]-2,3,4,9-tetrahydro-1H-pyrido[3,4-b]indole;
6-chloro-2-[4-(trifluoromethyl)pyrimidin-2-yl]-1-(2,3,4-trifluorophenyl)-2,3,4,9-tetrahydro-1H-pyrido[3,4-b]indole;
6-bromo-1-(4-methylphenyl)-2-[4-(trifluoromethyl)pyrimidin-2-yl]-2,3,4,9-tetrahydro-1H-pyrido[3,4-b]indole;
1-(4-methylphenyl)-6-nitro-2-[4-(trifluoromethyl)pyrimidin-2-yl]-2,3,4,9-tetrahydro-1H-pyrido[3,4-b]indole;
6-chloro-1-(2,3-difluoro-4-methylphenyl)-5-nitro-2-[4-(trifluoromethyl)pyrimidin-2-yl]-2,3,4,9-tetrahydro-1H-pyrido[3,4-b]indole;
6-chloro-1-(4-chloro-2-fluorophenyl)-2-[4-(trifluoromethyl)pyrimidin-2-yl]-2,3,4,9-tetrahydro-1H-pyrido[3,4-b]indole;
1-(4-bromo-2-fluorophenyl)-6-chloro-2-[4-(trifluoromethyl)pyrimidin-2-yl]-2,3,4,9-tetrahydro-1H-pyrido[3,4-b]indole;
6-chloro-1-[4-(4H-1,2,4-triazol-4-yl)phenyl]-2-[4-(trifluoromethyl)pyrimidin-2-yl]-2,3,4,9-tetrahydro-1H-pyrido[3,4-b]indole;
1-(4-bromo-2,3-difluorophenyl)-6-chloro-2-[4-(trifluoromethyl)pyrimidin-2-yl]-2,3,4,9-tetrahydro-1H-pyrido[3,4-b]indole;
1-(2,3-difluoro-4-methylphenyl)-6-methyl-2-[4-(trifluoromethyl)pyrimidin-2-yl]-2,3,4,9-tetrahydro-1H-pyrido[3,4-b]indole;
1-(2,3-difluoro-4-methylphenyl)-6-methyl-2-[4-(trifluoromethyl)pyrimidin-2-yl]-2,3,4,9-tetrahydro-1H-pyrido[3,4-b]indol-8-amine;
6-chloro-1-(2,3-difluoro-4-methylphenyl)-2-[4-(trifluoromethyl)pyrimidin-2-yl]-2,3,4,9-tetrahydro-1H-pyrido[3,4-b]indol-5-amine;
6-chloro-1-[2-fluoro-4-(1H-imidazol-1-yl)phenyl]-2-(pyrimidin-2-yl)-2,3,4,9-tetrahydro-1H-pyrido[3,4-b]indole;
6-chloro-1-[2-fluoro-4-(1H-imidazol-1-yl)phenyl]-2-[4-(trifluoromethyl)pyrimidin-2-yl]-2,3,4,9-tetrahydro-1H-pyrido[3,4-b]indole;
1-(2,3-difluoro-4-methylphenyl)-6-methyl-2-[4-(trifluoromethyl)pyrimidin-2-yl]-2,3,4,9-tetrahydro-1H-pyrido[3,4-b]indol-8-ol;
6-chloro-1-[2-fluoro-4-(morpholin-4-yl)phenyl]-2-[4-(trifluoromethyl)pyrimidin-2-yl]-2,3,4,9-tetrahydro-1H-pyrido[3,4-b]indole;
4-{6-chloro-2-[4-(trifluoromethyl)pyrimidin-2-yl]-2,3,4,9-tetrahydro-1H-pyrido[3,4-b]indol-1-yl}-2,3-difluorobenzonitrile;
6-chloro-1-[2,3-difluoro-4-(morpholin-4-yl)phenyl]-2-[4-(trifluoromethyl)pyrimidin-2-yl]-2,3,4,9-tetrahydro-1H-pyrido[3,4-b]indole;
butyl 2-[6-chloro-1-(4-methylphenyl)-1,3,4,9-tetrahydro-2H-pyrido[3,4-b]indol-2-yl]pyrimidine-4-carboxylate;
butyl 2-[6-chloro-1-(2,3-difluoro-4-methylphenyl)-1,3,4,9-tetrahydro-2H-pyrido[3,4-b]indol-2-yl]pyrimidine-4-carboxylate;
6-chloro-1-(2,3-difluoro-4-methylphenyl)-2-[4-(trifluoromethyl)pyrimidin-2-yl]-2,3,4,9-tetrahydro-1H-pyrido[3,4-b]indol-7-amine;
1-(4-bromo-2,3-difluorophenyl)-6-chloro-2-(4,6-dimethyl-1,3,5-triazin-2-yl)-2,3,4,9-tetrahydro-1H-pyrido[3,4-b]indole;
6-chloro-1-[2,3-difluoro-4-(morpholin-4-yl)phenyl]-2-(4,6-dimethyl-1,3,5-triazin-2-yl)-2,3,4,9-tetrahydro-1H-pyrido[3,4-b]indole;
6-chloro-1-[2,3-difluoro-4-(1H-imidazol-1-yl)phenyl]-2-[4-(trifluoromethyl)pyrimidin-2-yl]-2,3,4,9-tetrahydro-1H-pyrido[3,4-b]indole;
6-chloro-2-(4,6-dimethylpyrimidin-2-yl)-1-(4-methylphenyl)-2,3,4,9-tetrahydro-1H-pyrido[3,4-b]indole;
1-(4-methylphenyl)-6-(2H-1,2,3-triazol-2-yl)-2-[4-(trifluoromethyl)pyrimidin-2-yl]-2,3,4,9-tetrahydro-1H-pyrido[3,4-b]indole;
1-[2-(morpholin-4-yl)pyrimidin-5-yl]-6-(2H-1,2,3-triazol-2-yl)-2-[4-(trifluoromethyl)pyrimidin-2-yl]-2,3,4,9-tetrahydro-1H-pyrido[3,4-b]indole;
6-chloro-2-(4-chloro-6-methyl-1,3,5-triazin-2-yl)-1-[2-(morpholin-4-yl)pyrimidin-5-yl]-2,3,4,9-tetrahydro-1H-pyrido[3,4-b]indole;
4-{6-chloro-1-[2-(morpholin-4-yl)pyrimidin-5-yl]-1,3,4,9-tetrahydro-2H-pyrido[3,4-b]indol-2-yl}-N,N,6-trimethyl-1,3,5-triazin-2-amine;
6-ethenyl-1-(4-methylphenyl)-2-[4-(trifluoromethyl)pyrimidin-2-yl]-2,3,4,9-tetrahydro-1H-pyrido[3,4-b]indole;
6-chloro-2-(4,6-dimethyl-1,3,5-triazin-2-yl)-1-(4-methylphenyl)-2,3,4,9-tetrahydro-1H-pyrido[3,4-b]indol-8-amine;
6-chloro-2-(4,6-dimethyl-1,3,5-triazin-2-yl)-1-[2-(morpholin-4-yl)pyrimidin-5-yl]-2,3,4,9-tetrahydro-1H-pyrido[3,4-b]indole;
6-chloro-2-(4-methyl-1,3,5-triazin-2-yl)-1-[2-(morpholin-4-yl)pyrimidin-5-yl]-2,3,4,9-tetrahydro-1H-pyrido[3,4-b]indole;
6-chloro-2-(4-methoxy-6-methyl-1,3,5-triazin-2-yl)-1-[2-(morpholin-4-yl)pyrimidin-5-yl]-2,3,4,9-tetrahydro-1H-pyrido[3,4-b]indole;
6-chloro-2-[4-methyl-6-(morpholin-4-yl)-1,3,5-triazin-2-yl]-1-[2-(morpholin-4-yl)pyrimidin-5-yl]-2,3,4,9-tetrahydro-1H-pyrido[3,4-b]indole;
4-(6-chloro-1-[2-(morpholin-4-yl)pyrimidin-5-yl]-1,3,4,9-tetrahydro-2H-pyrido[3,4-b]indol-2-yl)-N,N-dimethyl-1,3,5-triazin-2-amine;
6-bromo-1-[2-(morpholin-4-yl)pyrimidin-5-yl]-2-[4-(trifluoromethyl)pyrimidin-2-yl]-2,3,4,9-tetrahydro-1H-pyrido[3,4-b]indole;
6-bromo-2-(4,6-dimethyl-1,3,5-triazin-2-yl)-1-[2-(morpholin-4-yl)pyrimidin-5-yl]-2,3,4,9-tetrahydro-1H-pyrido[3,4-b]indole;
2-(4,6-dimethyl-1,3,5-triazin-2-yl)-1-[2-(morpholin-4-yl)pyrimidin-5-yl]-6-(2H-1,2,3-triazol-2-yl)-2,3,4,9-tetrahydro-1H-pyrido[3,4-b]indole;
1-[2-(morpholin-4-yl)pyrimidin-5-yl]-6-(1H-pyrazol-1-yl)-2-[4-(trifluoromethyl)pyrimidin-2-yl]-2,3,4,9-tetrahydro-1H-pyrido[3,4-b]indole;
6-methoxy-1-[2-(morpholin-4-yl)pyrimidin-5-yl]-2-[4-(trifluoromethyl)pyrimidin-2-yl]-2,3,4,9-tetrahydro-1H-pyrido[3,4-b]indole;
6-chloro-1-[6-(morpholin-4-yl)pyridin-3-yl]-2-[4-(trifluoromethyl)pyrimidin-2-yl]-2,3,4,9-tetrahydro-1H-pyrido[3,4-b]indole;
6-chloro-1-(2,3-difluoro-4-methylphenyl)-2-(4,6-dimethyl-1,3,5-triazin-2-yl)-2,3,4,9-tetrahydro-1H-pyrido[3,4-b]indol-8-amine;
1-(2,3-difluoro-4-methylphenyl)-6-nitro-2-[4-(trifluoromethyl)pyrimidin-2-yl]-2,3,4,9-tetrahydro-1H-pyrido[3,4-b]indole;
2-[6-chloro-1-(2,3-difluoro-4-methylphenyl)-1,3,4,9-tetrahydro-2H-pyrido[3,4-b]indol-2-yl]-N,N-dimethylpyrimidin-4-amine;
2-[6-chloro-1-(4-methylphenyl)-1,3,4,9-tetrahydro-2H-pyrido[3,4-b]indol-2-yl]-N,N-dimethylpyrimidin-4-amine;
6-chloro-2-(4,6-dimethylpyrimidin-2-yl)-1-[2-(morpholin-4-yl)pyrimidin-5-yl]-2,3,4,9-tetrahydro-1H-pyrido[3,4-b]indole;
6-chloro-2-(4,6-dimethyl-1,3,5-triazin-2-yl)-1-[2-(morpholin-4-yl)pyrimidin-5-yl]-2,3,4,9-tetrahydro-1H-pyrido[3,4-b]indol-8-amine;
6-chloro-1-[2-(morpholin-4-yl)pyrimidin-5-yl]-2-[4-(trifluoromethyl)pyrimidin-2-yl]-2,3,4,9-tetrahydro-1H-pyrido[3,4-b]indol-8-amine;
6-chloro-1-[2-(morpholin-4-yl)pyrimidin-5-yl]-2-[4-(trifluoromethyl)pyrimidin-2-yl]-2,3,4,9-tetrahydro-1H-pyrido[3,4-b]indol-7-amine;
6-chloro-2-(4-methylpyrimidin-2-yl)-1-[2-(morpholin-4-yl)pyrimidin-5-yl]-2,3,4,9-tetrahydro-1H-pyrido[3,4-b]indole;
2-(4,6-dimethyl-1,3,5-triazin-2-yl)-1-[2-(morpholin-4-yl)pyrimidin-5-yl]-6-nitro-2,3,4,9-tetrahydro-1H-pyrido[3,4-b]indole;
1-[2-(morpholin-4-yl)pyrimidin-5-yl]-6-(1H-pyrrol-1-yl)-2-[4-(trifluoromethyl)pyrimidin-2-yl]-2,3,4,9-tetrahydro-1H-pyrido[3,4-b]indole;
N,N-dibutyl-4-{6-chloro-1-[2-(morpholin-4-yl)pyrimidin-5-yl]-1,3,4,9-tetrahydro-2H-pyrido[3,4-b]indol-2-yl}-6-methyl-1,3,5-triazin-2-amine;
1-[6-(morpholin-4-yl)pyridin-3-yl]-6-(2H-1,2,3-triazol-2-yl)-2-[4-(trifluoromethyl)pyrimidin-2-yl]-2,3,4,9-tetrahydro-1H-pyrido[3,4-b]indole;
N-butyl-4-{6-chloro-1-[2-(morpholin-4-yl)pyrimidin-5-yl]-1,3,4,9-tetrahydro-2H-pyrido[3,4-b]indol-2-yl}-N,6-dimethyl-1,3,5-triazin-2-amine;
2-(4-butoxy-6-methyl-1,3,5-triazin-2-yl)-6-chloro-1-[2-(morpholin-4-yl)pyrimidin-5-yl]-2,3,4,9-tetrahydro-1H-pyrido[3,4-b]indole;
6-chloro-1-(2,6-dichloropyridin-3-yl)-2-[4-(trifluoromethyl)pyrimidin-2-yl]-2,3,4,9-tetrahydro-1H-pyrido[3,4-b]indole;
6-chloro-2-(4,6-dimethyl-1,3,5-triazin-2-yl)-1-[2-(morpholin-4-yl)pyrimidin-5-yl]-2,3,4,9-tetrahydro-1H-pyrido[3,4-b]indol-7-amine;
6-chloro-2-(4,6-dimethyl-1,3,5-triazin-2-yl)-1-[6-(morpholin-4-yl)pyridin-3-yl]-2,3,4,9-tetrahydro-1H-pyrido[3,4-b]indol-8-amine;
6-chloro-1-(4-methylphenyl)-2-(4-methyl-1,3,5-triazin-2-yl)-2,3,4,9-tetrahydro-1H-pyrido[3,4-b]indole;
1-[2-(morpholin-4-yl)pyrimidin-5-yl]-6-nitro-2-[4-(trifluoromethyl)pyrimidin-2-yl]-2,3,4,9-tetrahydro-1H-pyrido[3,4-b]indole;
6-chloro-2-(4,6-dimethyl-1,3,5-triazin-2-yl)-1-[6-(morpholin-4-yl)pyridin-3-yl]-2,3,4,9-tetrahydro-1H-pyrido[3,4-b]indole;
6-chloro-2-(4,6-dimethyl-1,3,5-triazin-2-yl)-1-[2-(morpholin-4-yl)pyrimidin-5-yl]-5-nitro-2,3,4,9-tetrahydro-1H-pyrido[3,4-b]indole;
6-chloro-1-[2-(morpholin-4-yl)pyrimidin-5-yl]-5-nitro-2-[4-(trifluoromethyl)pyrimidin-2-yl]-2,3,4,9-tetrahydro-1H-pyrido[3,4-b]indole;
6-chloro-2-(4,6-dimethyl-1,3,5-triazin-2-yl)-1-[2-(morpholin-4-yl)pyrimidin-5-yl]-2,3,4,9-tetrahydro-1H-pyrido[3,4-b]indol-5-amine;
6-chloro-1-[2-(morpholin-4-yl)pyrimidin-5-yl]-2-[4-(trifluoromethyl)pyrimidin-2-yl]-2,3,4,9-tetrahydro-1H-pyrido[3,4-b]indol-5-amine;
N,N-dimethyl-1-[2-(morpholin-4-yl)pyrimidin-5-yl]-2-[4-(trifluoromethyl)pyrimidin-2-yl]-2,3,4,9-tetrahydro-1H-pyrido[3,4-b]indol-6-amine;
1-(2,3-difluoro-4-methylphenyl)-N,N-dimethyl-2-[4-(trifluoromethyl)pyrimidin-2-yl]-2,3,4,9-tetrahydro-1H-pyrido[3,4-b]indol-6-amine;
2-(4,6-dimethyl-1,3,5-triazin-2-yl)-N,N-dimethyl-1-[2-(morpholin-4-yl)pyrimidin-5-yl]-2,3,4,9-tetrahydro-1H-pyrido[3,4-b]indol-6-amine;
6-chloro-1-(4-methylphenyl)-2-(4-methylpyrimidin-2-yl)-2,3,4,9-tetrahydro-1H-pyrido[3,4-b]indole;
2-(4,6-dimethyl-1,3,5-triazin-2-yl)-1-[2-(morpholin-4-yl)pyrimidin-5-yl]-6-nitro-2,3,4,9-tetrahydro-1H-pyrido[3,4-b]indol-8-ol;
6-chloro-2-[4-(difluoromethyl)pyrimidin-2-yl]-1-[2-(morpholin-4-yl)pyrimidin-5-yl]-2,3,4,9-tetrahydro-1H-pyrido[3,4-b]indole;
6-chloro-1-[2-(2-oxa-5-azabicyclo[2.2.1]heptan-5-yl)pyrimidin-5-yl]-2-[4-(trifluoromethyl)pyrimidin-2-yl]-2,3,4,9-tetrahydro-1H-pyrido[3,4-b]indole;
6-chloro-2-(4,6-dimethyl-1,3,5-triazin-2-yl)-1-[2-(2-oxa-5-azabicyclo[2.2.1]heptan-5-yl)pyrimidin-5-yl]-2,3,4,9-tetrahydro-1H-pyrido[3,4-b]indole;
6-chloro-1-[2-(3-oxa-8-azabicyclo[3.2.1]octan-8-yl)pyrimidin-5-yl]-2-[4-(trifluoromethyl)pyrimidin-2-yl]-2,3,4,9-tetrahydro-1H-pyrido[3,4-b]indole;
6-chloro-2-(4,6-dimethyl-1,3,5-triazin-2-yl)-1-[2-(3-oxa-8-azabicyclo[3.2.1]octan-8-yl)pyrimidin-5-yl]-2,3,4,9-tetrahydro-1H-pyrido[3,4-b]indole;
6-chloro-2-(4,6-dimethyl-1,3,5-triazin-2-yl)-1-[6-(morpholin-4-yl)pyridin-3-yl]-2,3,4,9-tetrahydro-1H-pyrido[3,4-b]indol-8-ol;
2-[4,6-bis(trifluoromethyl)pyrimidin-2-yl]-6-chloro-1-(2,3-difluoro-4-methylphenyl)-2,3,4,9-tetrahydro-1H-pyrido[3,4-b]indole;
6-chloro-1-(2-fluorophenyl)-2-[4-(trifluoromethyl)pyrimidin-2-yl]-2,3,4,9-tetrahydro-1H-pyrido[3,4-b]indole;
6-chloro-1-(2,3-difluoro-4-methylphenyl)-2-(4,6-dimethylpyrimidin-2-yl)-2,3,4,9-tetrahydro-1H-pyrido[3,4-b]indole;
6-chloro-1-(4-methylphenyl)-2-[4-(trifluoromethyl)pyrimidin-2-yl]-2,3,4,9-tetrahydro-1H-pyrido[3,4-b]indole;
1-[2-(azetidin-1-yl)pyrimidin-5-yl]-6-chloro-2-[4-(trifluoromethyl)pyrimidin-2-yl]-2,3,4,9-tetrahydro-1H-pyrido[3,4-b]indole;
1-[2-(azetidin-1-yl)pyrimidin-5-yl]-6-chloro-2-(4,6-dimethyl-1,3,5-triazin-2-yl)-2,3,4,9-tetrahydro-1H-pyrido[3,4-b]indole;
6-chloro-2-(4,6-dimethyl-1,3,5-triazin-2-yl)-1-[2-(methylsulfanyl)pyrimidin-5-yl]-2,3,4,9-tetrahydro-1H-pyrido[3,4-b]indole;
(1S)-6-chloro-1-(2,3-difluoro-4-methylphenyl)-2-(4,6-dimethyl-1,3,5-triazin-2-yl)-2,3,4,9-tetrahydro-1H-pyrido[3,4-b]indole;
6-chloro-1-(2,3-difluoro-4-methylphenyl)-2-(4,6-dimethyl-1,3,5-triazin-2-yl)-2,3,4,9-tetrahydro-1H-pyrido[3,4-b]indol-8-ol;
6-chloro-1-(4-chlorophenyl)-2-[4-(trifluoromethyl)pyrimidin-2-yl]-2,3,4,9-tetrahydro-1H-pyrido[3,4-b]indole;
6-chloro-2-(4,6-dimethylpyrimidin-2-yl)-1-[6-(morpholin-4-yl)pyridin-3-yl]-2,3,4,9-tetrahydro-1H-pyrido[3,4-b]indole;
6-chloro-2-(4-methylpyrimidin-2-yl)-1-[6-(morpholin-4-yl)pyridin-3-yl]-2,3,4,9-tetrahydro-1H-pyrido[3,4-b]indol-8-amine;
6-chloro-2-(4-methylpyrimidin-2-yl)-1-[6-(morpholin-4-yl)pyridin-3-yl]-2,3,4,9-tetrahydro-1H-pyrido[3,4-b]indole;
2-(6-chloro-2-[4-(trifluoromethyl)pyrimidin-2-yl]-2,3,4,9-tetrahydro-1H-pyrido[3,4-b]indol-1-yl)phenol;
6-chloro-1-(3-fluoro-4-methylphenyl)-2-[4-(trifluoromethyl)pyrimidin-2-yl]-2,3,4,9-tetrahydro-1H-pyrido[3,4-b]indole;
6-chloro-2-(4,6-dimethyl-1,3,5-triazin-2-yl)-1-(4-methylphenyl)-2,3,4,9-tetrahydro-1H-pyrido[3,4-b]indole;
2-{6-chloro-1-[6-(morpholin-4-yl)pyridin-3-yl]-1,3,4,9-tetrahydro-2H-pyrido[3,4-b]indol-2-yl}pyrimidine-4-carbonitrile;
2-[4,6-bis(trifluoromethyl)pyrimidin-2-yl]-6-chloro-1-[6-(morpholin-4-yl)pyridin-3-yl]-2,3,4,9-tetrahydro-1H-pyrido[3,4-b]indole;
2-[4,6-bis(trifluoromethyl)pyrimidin-2-yl]-6-chloro-1-[2-(morpholin-4-yl)pyrimidin-5-yl]-2,3,4,9-tetrahydro-1H-pyrido[3,4-b]indole;
2-{6-chloro-1-[2-(morpholin-4-yl)pyrimidin-5-yl]-1,3,4,9-tetrahydro-2H-pyrido[3,4-b]indol-2-yl}pyrimidine-4-carbonitrile;
2-[4,6-bis(trifluoromethyl)pyrimidin-2-yl]-6-chloro-1-(4-methylphenyl)-2,3,4,9-tetrahydro-1H-pyrido[3,4-b]indole;
6-chloro-1-(4-methylphenyl)-2-[4-methyl-6-(trifluoromethyl)pyrimidin-2-yl]-2,3,4,9-tetrahydro-1H-pyrido[3,4-b]indole;
2-{6-chloro-1-[2-(morpholin-4-yl)pyrimidin-5-yl]-1,3,4,9-tetrahydro-2H-pyrido[3,4-b]indol-2-yl}-6-methylpyrimidine-4-carbonitrile;
2-[6-chloro-1-(2,3-difluoro-4-methylphenyl)-1,3,4,9-tetrahydro-2H-pyrido[3,4-b]indol-2-yl]pyrimidine-4-carbonitrile;
6-chloro-2-(5-fluoropyrimidin-2-yl)-1-[2-(morpholin-4-yl)pyrimidin-5-yl]-2,3,4,9-tetrahydro-1H-pyrido[3,4-b]indole;
6-chloro-2-(5-fluoro-4-methylpyrimidin-2-yl)-1-[6-(morpholin-4-yl)pyridin-3-yl]-2,3,4,9-tetrahydro-1H-pyrido[3,4-b]indole;
6-chloro-2-(5-fluoro-4-methylpyrimidin-2-yl)-1-[2-(morpholin-4-yl)pyrimidin-5-yl]-2,3,4,9-tetrahydro-1H-pyrido[3,4-b]indole;
6-chloro-2-(5-fluoro-4,6-dimethylpyrimidin-2-yl)-1-[6-(morpholin-4-yl)pyridin-3-yl]-2,3,4,9-tetrahydro-1H-pyrido[3,4-b]indole;
6-chloro-2-(5-fluoro-4,6-dimethylpyrimidin-2-yl)-1-[2-(morpholin-4-yl)pyrimidin-5-yl]-2,3,4,9-tetrahydro-1H-pyrido[3,4-b]indole;
6-chloro-1-[2-(4-methylpiperazin-1-yl)pyrimidin-5-yl]-2-[4-(trifluoromethyl)pyrimidin-2-yl]-2,3,4,9-tetrahydro-1H-pyrido[3,4-b]indole;
6-chloro-2-(4,6-dimethyl-1,3,5-triazin-2-yl)-1-[2-(morpholin-4-yl)pyrimidin-5-yl]-2,3,4,9-tetrahydro-1H-pyrido[3,4-b]indol-8-ol;
6-chloro-1-(3-fluorophenyl)-2-[4-(trifluoromethyl)pyrimidin-2-yl]-2,3,4,9-tetrahydro-1H-pyrido[3,4-b]indole;
6-chloro-2-[4-methyl-6-(trifluoromethyl)pyrimidin-2-yl]-1-[6-(morpholin-4-yl)pyridin-3-yl]-2,3,4,9-tetrahydro-1H-pyrido[3,4-b]indole;
6-chloro-1-{6-[(1R,4R)-2-oxa-5-azabicyclo[2.2.1]heptan-5-yl]pyridin-3-yl}-2-[4-(trifluoromethyl)pyrimidin-2-yl]-2,3,4,9-tetrahydro-1H-pyrido[3,4-b]indole;
6-chloro-2-[4-methyl-6-(trifluoromethyl)pyrimidin-2-yl]-1-[2-(morpholin-4-yl)pyrimidin-5-yl]-2,3,4,9-tetrahydro-1H-pyrido[3,4-b]indole;
6-chloro-1-{6-[(1S,4S)-2-oxa-5-azabicyclo[2.2.1]heptan-5-yl]pyridin-3-yl}-2-[4-(trifluoromethyl)pyrimidin-2-yl]-2,3,4,9-tetrahydro-1H-pyrido[3,4-b]indole;
6-chloro-2-(4,6-dimethyl-1,3,5-triazin-2-yl)-1-{6-[(1R,4R)-2-oxa-5-azabicyclo[2.2.1]heptan-5-yl]pyridin-3-yl}-2,3,4,9-tetrahydro-1H-pyrido[3,4-b]indole;
6-chloro-2-(4,6-dimethyl-1,3,5-triazin-2-yl)-1-{6-[(1S,4S)-2-oxa-5-azabicyclo[2.2.1]heptan-5-yl]pyridin-3-yl}-2,3,4,9-tetrahydro-1H-pyrido[3,4-b]indole;
2-[6-chloro-1-(4-methylphenyl)-1,3,4,9-tetrahydro-2H-pyrido[3,4-b]indol-2-yl]pyrimidine-4-carbonitrile;
2-[6-chloro-1-(4-methylphenyl)-1,3,4,9-tetrahydro-2H-pyrido[3,4-b]indol-2-yl]-6-methylpyrimidine-4-carbonitrile;
6-chloro-2-(4,6-dimethyl-1,3,5-triazin-2-yl)-1-[2-(4-methylpiperazin-1-yl)pyrimidin-5-yl]-2,3,4,9-tetrahydro-1H-pyrido[3,4-b]indole;
2-[4,6-bis(trifluoromethyl)pyrimidin-2-yl]-6-chloro-1-[2-(4-methylpiperazin-1-yl)pyrimidin-5-yl]-2,3,4,9-tetrahydro-1H-pyrido[3,4-b]indole;
6-chloro-2-(5-fluoropyrimidin-2-yl)-1-[6-(morpholin-4-yl)pyridin-3-yl]-2,3,4,9-tetrahydro-1H-pyrido[3,4-b]indole;
6-chloro-1-[2-(morpholin-4-yl)pyrimidin-5-yl]-2-[5-(trifluoromethyl)-1,2,4-triazin-3-yl]-2,3,4,9-tetrahydro-1H-pyrido[3,4-b]indole;
6-chloro-2-(5-methyl-1,2,4-triazin-3-yl)-1-[2-(morpholin-4-yl)pyrimidin-5-yl]-2,3,4,9-tetrahydro-1H-pyrido[3,4-b]indole;
2-[(1S)-6-chloro-1-(2,3-difluoro-4-methylphenyl)-1,3,4,9-tetrahydro-2H-pyrido[3,4-b]indol-2-yl]-5-fluoropyrimidine-4-carbonitrile;
(1S)-2-[4,6-bis(trifluoromethyl)-1,3,5-triazin-2-yl]-6-chloro-1-(4-methylphenyl)-2,3,4,9-tetrahydro-1H-pyrido[3,4-b]indole;
(1S)-2-[4,6-bis(trifluoromethyl)-1,3,5-triazin-2-yl]-6-chloro-1-(2,3-difluoro-4-methylphenyl)-2,3,4,9-tetrahydro-1H-pyrido[3,4-b]indole;
2-[4,6-bis(trifluoromethyl)-1,3,5-triazin-2-yl]-6-chloro-1-[2-(morpholin-4-yl)pyrimidin-5-yl]-2,3,4,9-tetrahydro-1H-pyrido[3,4-b]indole;
(1S)-6-chloro-1-(2,3-difluoro-4-methylphenyl)-2-[4-(methylsulfanyl)-6-(trifluoromethyl)-1,3,5-triazin-2-yl]-2,3,4,9-tetrahydro-1H-pyrido[3,4-b]indole;
4-[(1S)-6-chloro-1-(2,3-difluoro-4-methylphenyl)-1,3,4,9-tetrahydro-2H-pyrido[3,4-b]indol-2-yl]-6-(trifluoromethyl)-1,3,5-triazine-2-carbonitrile;
6-chloro-1-[2-(2-oxa-6-azaspiro[3.3]heptan-6-yl)pyrimidin-5-yl]-2-[4-(trifluoromethyl)pyrimidin-2-yl]-2,3,4,9-tetrahydro-1H-pyrido[3,4-b]indole;
2-[(1S)-6-chloro-1-(4-methylphenyl)-1,3,4,9-tetrahydro-2H-pyrido[3,4-b]indol-2-yl]-5-fluoropyrimidine-4-carbonitrile;
(1S)-6-chloro-2-(5-fluoropyrimidin-2-yl)-1-(4-methylphenyl)-2,3,4,9-tetrahydro-1H-pyrido[3,4-b]indole;
(1S)-6-chloro-2-(5-fluoro-4-methylpyrimidin-2-yl)-1-(4-methylphenyl)-2,3,4,9-tetrahydro-1H-pyrido[3,4-b]indole;
(1S)-6-chloro-2-(5-fluoro-4,6-dimethylpyrimidin-2-yl)-1-(4-methylphenyl)-2,3,4,9-tetrahydro-1H-pyrido[3,4-b]indole;
2-{(1S)-6-chloro-1-[2-(morpholin-4-yl)pyrimidin-5-yl]-1,3,4,9-tetrahydro-2H-pyrido[3,4-b]indol-2-yl}-5-fluoropyrimidine-4-carbonitrile;
2-[(1S)-6-chloro-1-(4-methylphenyl)-1,3,4,9-tetrahydro-2H-pyrido[3,4-b]indol-2-yl]pyrimidine-4,6-dicarbonitrile;
2-{(1S)-6-chloro-1-[2-(morpholin-4-yl)pyrimidin-5-yl]-1,3,4,9-tetrahydro-2H-pyrido[3,4-b]indol-2-yl}pyrimidine-4,6-dicarbonitrile;
4-[(1S)-6-chloro-1-(4-methylphenyl)-1,3,4,9-tetrahydro-2H-pyrido[3,4-b]indol-2-yl]-1,3,5-triazine-2-carbonitrile;
2-((S)-6-chloro-1-[6-(morpholin-4-yl)pyridin-3-yl]-1,3,4,9-tetrahydro-2H-pyrido[3,4-b]indol-2-yl)-5-fluoropyrimidine-4-carbonitrile;
6-chloro-1-(2,3-difluoro-4-methylphenyl)-2-(5-fluoropyrimidin-2-yl)-2,3,4,9-tetrahydro-1H-pyrido[3,4-b]indole;
(1S)-6-chloro-2-(4,6-dimethyl-1,3,5-triazin-2-yl)-1-(4-methylphenyl)-2,3,4,9-tetrahydro-1H-pyrido[3,4-b]indole;
6-chloro-1-(2,3-difluoro-4-methylphenyl)-2-[5-(trifluoromethyl)-1,2,4-triazin-3-yl]-2,3,4,9-tetrahydro-1H-pyrido[3,4-b]indole;
2-[4,6-bis(trifluoromethyl)-1,3,5-triazin-2-yl]-6-chloro-1-[6-(morpholin-4-yl)pyridin-3-yl]-2,3,4,9-tetrahydro-1H-pyrido[3,4-b]indole;
(1S)-2-[4,6-bis(trifluoromethyl)-1,3,5-triazin-2-yl]-6-chloro-1-[6-(morpholin-4-yl)pyridin-3-yl]-2,3,4,9-tetrahydro-1H-pyrido[3,4-b]indole;
(1S)-2-[4,6-bis(trifluoromethyl)-1,3,5-triazin-2-yl]-6-chloro-1-[2-(morpholin-4-yl)pyrimidin-5-yl]-2,3,4,9-tetrahydro-1H-pyrido[3,4-b]indole;
(1S)-2-[4,6-bis(trifluoromethyl)pyrimidin-2-yl]-6-chloro-1-(2,3-difluoro-4-methylphenyl)-2,3,4,9-tetrahydro-1H-pyrido[3,4-b]indole;
(1S)-6-chloro-1-[2-(morpholin-4-yl)pyrimidin-5-yl]-2-[4-(trifluoromethyl)pyrimidin-2-yl]-2,3,4,9-tetrahydro-1H-pyrido[3,4-b]indole;
(1S)-2-[4,6-bis(trifluoromethyl)pyrimidin-2-yl]-6-chloro-1-[6-(morpholin-4-yl)pyridin-3-yl]-2,3,4,9-tetrahydro-1H-pyrido[3,4-b]indole;
(1S)-2-[4,6-bis(trifluoromethyl)pyrimidin-2-yl]-6-chloro-1-[2-(morpholin-4-yl)pyrimidin-5-yl]-2,3,4,9-tetrahydro-1H-pyrido[3,4-b]indole;
4-[(1S)-6-chloro-1-(4-methylphenyl)-1,3,4,9-tetrahydro-2H-pyrido[3,4-b]indol-2-yl]-6-methyl-1,3,5-triazine-2-carbonitrile;
(1S)-6-chloro-1-(2,3-difluoro-4-methylphenyl)-2-(5-fluoro-4,6-dimethylpyrimidin-2-yl)-2,3,4,9-tetrahydro-1H-pyrido[3,4-b]indole;
2-[(1S)-6-chloro-1-(2,3-difluoro-4-methylphenyl)-1,3,4,9-tetrahydro-2H-pyrido[3,4-b]indol-2-yl]-5-fluoro-6-methylpyrimidine-4-carbonitrile;
4-((1S)-6-chloro-1-[2-(morpholin-4-yl)pyrimidin-5-yl]-1,3,4,9-tetrahydro-2H-pyrido[3,4-b]indol-2-yl)-1,3,5-triazine-2-carbonitrile;
4-{(1S)-6-chloro-1-[2-(morpholin-4-yl)pyrimidin-5-yl]-1,3,4,9-tetrahydro-2H-pyrido[3,4-b]indol-2-yl}-6-methyl-1,3,5-triazine-2-carbonitrile;
2-[(1S)-6-chloro-1-(2,3-difluoro-4-methylphenyl)-1,3,4,9-tetrahydro-2H-pyrido[3,4-b]indol-2-yl]-6-(trifluoromethyl)pyrimidine-4-carbonitrile;
2-[(1S)-6-chloro-1-(4-methylphenyl)-1,3,4,9-tetrahydro-2H-pyrido[3,4-b]indol-2-yl]-5-fluoro-6-methylpyrimidine-4-carbonitrile;
(1S)-6-chloro-1-(4-methylphenyl)-2-[4-(trifluoromethyl)-1,3,5-triazin-2-yl]-2,3,4,9-tetrahydro-1H-pyrido[3,4-b]indole;
(1S)-2-[4,6-bis(trifluoromethyl)pyrimidin-2-yl]-6-chloro-1-[2-(4-methylpiperazin-1-yl)pyrimidin-5-yl]-2,3,4,9-tetrahydro-1H-pyrido[3,4-b]indole;
(1S)-2-[4,6-bis(trifluoromethyl)-1,3,5-triazin-2-yl]-6-chloro-1-[2-(4-methylpiperazin-1-yl)pyrimidin-5-yl]-2,3,4,9-tetrahydro-1H-pyrido[3,4-b]indole;
2-((1S)-6-chloro-1-[2-(morpholin-4-yl)pyrimidin-5-yl]-1,3,4,9-tetrahydro-2H-pyrido[3,4-b]indol-2-yl)-5-fluoro-6-methylpyrimidine-4-carbonitrile;
6-chloro-1-(4-fluorophenyl)-2-[4-(trifluoromethyl)pyrimidin-2-yl]-2,3,4,9-tetrahydro-1H-pyrido[3,4-b]indole;
6-chloro-1-(2,4-difluorophenyl)-2-[4-(trifluoromethyl)pyrimidin-2-yl]-2,3,4,9-tetrahydro-1H-pyrido[3,4-b]indole;
2-{(1S)-6-chloro-1-[6-(morpholin-4-yl)pyridin-3-yl]-1,3,4,9-tetrahydro-2H-pyrido[3,4-b]indol-2-yl}-5-fluoro-6-methylpyrimidine-4-carbonitrile;
2-[(1S)-6-chloro-1-(4-methylphenyl)-1,3,4,9-tetrahydro-2H-pyrido[3,4-b]indol-2-yl]-6-(trifluoromethyl)pyrimidine-4-carbonitrile;
4-[(1S)-6-chloro-1-(4-methylphenyl)-1,3,4,9-tetrahydro-2H-pyrido[3,4-b]indol-2-yl]-6-(trifluoromethyl)-1,3,5-triazin-2-amine;
4-[(1S)-6-chloro-1-(2,3-difluoro-4-methylphenyl)-1,3,4,9-tetrahydro-2H-pyrido[3,4-b]indol-2-yl]-6-(trifluoromethyl)-1,3,5-triazin-2-amine;
6-chloro-1-[6-(morpholin-4-yl)pyridin-3-yl]-2-[4-(morpholin-4-yl)-6-(trifluoromethyl)-1,3,5-triazin-2-yl]-2,3,4,9-tetrahydro-1H-pyrido[3,4-b]indole;
6-chloro-1-[6-(morpholin-4-yl)pyridin-3-yl]-2-[5-(trifluoromethyl)-1,2,4-triazin-3-yl]-2,3,4,9-tetrahydro-1H-pyrido[3,4-b]indole;
6-chloro-1-(4-methylphenyl)-2-[5-(trifluoromethyl)-1,2,4-triazin-3-yl]-2,3,4,9-tetrahydro-1H-pyrido[3,4-b]indole;
6-chloro-1-(2,3-difluoro-4-methylphenyl)-2-[6-(trifluoromethyl)-1,2,4-triazin-3-yl]-2,3,4,9-tetrahydro-1H-pyrido[3,4-b]indole;
6-chloro-1-[2-fluoro-6-(morpholin-4-yl)pyridin-3-yl]-2-[4-(trifluoromethyl)pyrimidin-2-yl]-2,3,4,9-tetrahydro-1H-pyrido[3,4-b]indole;
2-{(1S)-6-chloro-1-[6-(morpholin-4-yl)pyridin-3-yl]-1,3,4,9-tetrahydro-2H-pyrido[3,4-b]indol-2-yl}-6-(trifluoromethyl)pyrimidine-4-carbonitrile;
2-{(1S)-6-chloro-1-[2-(morpholin-4-yl)pyrimidin-5-yl]-1,3,4,9-tetrahydro-2H-pyrido[3,4-b]indol-2-yl}-6-(trifluoromethyl)pyrimidine-4-carbonitrile;
4-[(1S)-6-chloro-1-(2,3-difluoro-4-methylphenyl)-1,3,4,9-tetrahydro-2H-pyrido[3,4-b]indol-2-yl]-N,N-dimethyl-6-(trifluoromethyl)-1,3,5-triazin-2-amine;
(1S)-6-chloro-1-(2,3-difluoro-4-methylphenyl)-2-[4-(morpholin-4-yl)-6-(trifluoromethyl)-1,3,5-triazin-2-yl]-2,3,4,9-tetrahydro-1H-pyrido[3,4-b]indole;
6-chloro-1-(4-methylphenyl)-2-[4-methyl-6-(trifluoromethyl)-1,3,5-triazin-2-yl]-2,3,4,9-tetrahydro-1H-pyrido[3,4-b]indole;
6-chloro-1-(2,3-difluoro-4-methylphenyl)-2-[4-methyl-6-(trifluoromethyl)-1,3,5-triazin-2-yl]-2,3,4,9-tetrahydro-1H-pyrido[3,4-b]indole;
6-chloro-2-[4-methyl-6-(trifluoromethyl)-1,3,5-triazin-2-yl]-1-[6-(morpholin-4-yl)pyridin-3-yl]-2,3,4,9-tetrahydro-1H-pyrido[3,4-b]indole;
6-chloro-2-[4-methyl-6-(trifluoromethyl)-1,3,5-triazin-2-yl]-1-[2-(morpholin-4-yl)pyrimidin-5-yl]-2,3,4,9-tetrahydro-1H-pyrido[3,4-b]indole;
4-[(1S)-6-chloro-1-(4-methylphenyl)-1,3,4,9-tetrahydro-2H-pyrido[3,4-b]indol-2-yl]-N,N-dimethyl-6-(trifluoromethyl)-1,3,5-triazin-2-amine;
(1S)-6-chloro-1-(4-methylphenyl)-2-[4-(morpholin-4-yl)-6-(trifluoromethyl)-1,3,5-triazin-2-yl]-2,3,4,9-tetrahydro-1H-pyrido[3,4-b]indole;
4-{(1S)-6-chloro-1-[6-(morpholin-4-yl)pyridin-3-yl]-1,3,4,9-tetrahydro-2H-pyrido[3,4-b]indol-2-yl}-N,N-dimethyl-6-(trifluoromethyl)-1,3,5-triazin-2-amine;
4-((1S)-6-chloro-1-[2-(morpholin-4-yl)pyrimidin-5-yl]-1,3,4,9-tetrahydro-2H-pyrido[3,4-b]indol-2-yl)-N,N-dimethyl-6-(trifluoromethyl)-1,3,5-triazin-2-amine;
(1S)-6-chloro-1-[2-(morpholin-4-yl)pyrimidin-5-yl]-2-[4-(morpholin-4-yl)-6-(trifluoromethyl)-1,3,5-triazin-2-yl]-2,3,4,9-tetrahydro-1H-pyrido[3,4-b]indole;
(1S)-6-chloro-1-[6-(morpholin-4-yl)pyridin-3-yl]-2-[4-(morpholin-4-yl)-6-(trifluoromethyl)-1,3,5-triazin-2-yl]-2,3,4,9-tetrahydro-1H-pyrido[3,4-b]indole;
4-{6-chloro-2-[4-(trifluoromethyl)pyrimidin-2-yl]-2,3,4,9-tetrahydro-1H-pyrido[3,4-b]indol-1-yl}benzonitrile;
4-[6-chloro-2-(4,6-dimethyl-1,3,5-triazin-2-yl)-2,3,4,9-tetrahydro-1H-pyrido[3,4-b]indol-1-yl]benzonitrile;
6-chloro-1-(4-chlorophenyl)-2-(4,6-dimethyl-1,3,5-triazin-2-yl)-2,3,4,9-tetrahydro-1H-pyrido[3,4-b]indole;
6-chloro-2-(4,6-dimethyl-1,3,5-triazin-2-yl)-1-(4-fluorophenyl)-2,3,4,9-tetrahydro-1H-pyrido[3,4-b]indole;
6-chloro-1-(2,4-difluorophenyl)-2-(4,6-dimethyl-1,3,5-triazin-2-yl)-2,3,4,9-tetrahydro-1H-pyrido[3,4-b]indole;
6-chloro-2-(4,6-dimethyl-1,3,5-triazin-2-yl)-1-(2-fluoro-4-methylphenyl)-2,3,4,9-tetrahydro-1H-pyrido[3,4-b]indole;
4-[6-chloro-2-(4,6-dimethyl-1,3,5-triazin-2-yl)-2,3,4,9-tetrahydro-1H-pyrido[3,4-b]indol-1-yl]-3-fluorobenzonitrile;
2-[4,6-bis(trifluoromethyl)pyrimidin-2-yl]-1-[6-(morpholin-4-yl)pyridin-3-yl]-6-(2H-1,2,3-triazol-2-yl)-2,3,4,9-tetrahydro-1H-pyrido[3,4-b]indole;
2-[4,6-bis(trifluoromethyl)pyrimidin-2-yl]-6-(4-methyl-2H-1,2,3-triazol-2-yl)-1-[6-(morpholin-4-yl)pyridin-3-yl]-2,3,4,9-tetrahydro-1H-pyrido[3,4-b]indole;
(1S)-6-chloro-2-[4-(3,3-difluoroazetidin-1-yl)pyrimidin-2-yl]-1-[2-(morpholin-4-yl)pyrimidin-5-yl]-2,3,4,9-tetrahydro-1H-pyrido[3,4-b]indole;
(1S)-6-chloro-2-[4-(3,3-difluoroazetidin-1-yl)-6-methyl-1,3,5-triazin-2-yl]-1-[2-(morpholin-4-yl)pyrimidin-5-yl]-2,3,4,9-tetrahydro-1H-pyrido[3,4-b]indole;
6-chloro-1-[2-(morpholin-4-yl)pyrimidin-5-yl]-2-[6-(trifluoromethyl)-1,2,4-triazin-3-yl]-2,3,4,9-tetrahydro-1H-pyrido[3,4-b]indole;
6-chloro-2-(4,6-dimethyl-1,3,5-triazin-2-yl)-1-[2-fluoro-6-(morpholin-4-yl)pyridin-3-yl]-2,3,4,9-tetrahydro-1H-pyrido[3,4-b]indole;
(1S)-6-chloro-1-[2-(morpholin-4-yl)pyrimidin-5-yl]-2-[4-(trifluoromethyl)-1,3,5-triazin-2-yl]-2,3,4,9-tetrahydro-1H-pyrido[3,4-b]indole;
(1S)-6-chloro-1-(2,3-difluoro-4-methylphenyl)-2-[4-(trifluoromethyl)-1,3,5-triazin-2-yl]-2,3,4,9-tetrahydro-1H-pyrido[3,4-b]indole;
(1S)-6-chloro-1-[6-(morpholin-4-yl)pyridin-3-yl]-2-[4-(trifluoromethyl)-1,3,5-triazin-2-yl]-2,3,4,9-tetrahydro-1H-pyrido[3,4-b]indole;
(1S)-2-(5-bromopyrimidin-2-yl)-6-chloro-1-(2,3-difluoro-4-methylphenyl)-2,3,4,9-tetrahydro-1H-pyrido[3,4-b]indole;
(1S)-6-chloro-1-(2,3-difluoro-4-methylphenyl)-2-[4-methyl-6-(trifluoromethyl)-1,3,5-triazin-2-yl]-2,3,4,9-tetrahydro-1H-pyrido[3,4-b]indole;
(1S)-6-chloro-2-[4-methyl-6-(trifluoromethyl)-1,3,5-triazin-2-yl]-1-[6-(morpholin-4-yl)pyridin-3-yl]-2,3,4,9-tetrahydro-1H-pyrido[3,4-b]indole;
(1S)-6-chloro-2-[4-methyl-6-(trifluoromethyl)-1,3,5-triazin-2-yl]-1-[2-(morpholin-4-yl)pyrimidin-5-yl]-2,3,4,9-tetrahydro-1H-pyrido[3,4-b]indole;
(1S)-6-chloro-1-(4-methylphenyl)-2-[4-methyl-6-(trifluoromethyl)-1,3,5-triazin-2-yl]-2,3,4,9-tetrahydro-1H-pyrido[3,4-b]indole;
6-chloro-1-(4-chloro-2-fluorophenyl)-2-(4,6-dimethyl-1,3,5-triazin-2-yl)-2,3,4,9-tetrahydro-1H-pyrido[3,4-b]indole;
6-chloro-1-(2,3-difluoro-4-methylphenyl)-2-(4-phenylpyrimidin-2-yl)-2,3,4,9-tetrahydro-1H-pyrido[3,4-b]indole;
(1S)-2-(5-bromopyrimidin-2-yl)-6-chloro-1-[2-(morpholin-4-yl)pyrimidin-5-yl]-2,3,4,9-tetrahydro-1H-pyrido[3,4-b]indole;
(1S)-6-chloro-1-(2,3-difluoro-4-methylphenyl)-2-(5-phenylpyrimidin-2-yl)-2,3,4,9-tetrahydro-1H-pyrido[3,4-b]indole;
(1S)-6-chloro-1-[2-(morpholin-4-yl)pyrimidin-5-yl]-2-(5-phenylpyrimidin-2-yl)-2,3,4,9-tetrahydro-1H-pyrido[3,4-b]indole;
(1S)-6-chloro-1-[6-(morpholin-4-yl)pyridin-3-yl]-2-[4-(trifluoromethyl)pyrimidin-2-yl]-2,3,4,9-tetrahydro-1H-pyrido[3,4-b]indole;
(1S)-6-chloro-1-[2-(4-methylpiperazin-1-yl)pyrimidin-5-yl]-2-[4-(methylsulfanyl)-6-(trifluoromethyl)-1,3,5-triazin-2-yl]-2,3,4,9-tetrahydro-1H-pyrido[3,4-b]indole;
6-chloro-1-[2-(morpholin-4-yl)pyrimidin-5-yl]-2-(4-phenylpyrimidin-2-yl)-2,3,4,9-tetrahydro-1H-pyrido[3,4-b]indole;
(1S)-6-chloro-1-(2,3-difluoro-4-methylphenyl)-2-[4-fluoro-6-(4-methylpiperazin-1-yl)-1,3,5-triazin-2-yl]-2,3,4,9-tetrahydro-1H-pyrido[3,4-b]indole;
(1S)-6-chloro-1-(2,3-difluoro-4-methylphenyl)-2-[4-(4-methylpiperazin-1-yl)-6-(trifluoromethyl)-1,3,5-triazin-2-yl]-2,3,4,9-tetrahydro-1H-pyrido[3,4-b]indole;
(1S)-6-chloro-2-[4-(4-methylpiperazin-1-yl)-6-(trifluoromethyl)-1,3,5-triazin-2-yl]-1-[2-(morpholin-4-yl)pyrimidin-5-yl]-2,3,4,9-tetrahydro-1H-pyrido[3,4-b]indole;
(1S)-6-chloro-1-[2-(4-methylpiperazin-1-yl)pyrimidin-5-yl]-2-[4-(4-methylpiperazin-1-yl)-6-(trifluoromethyl)-1,3,5-triazin-2-yl]-2,3,4,9-tetrahydro-1H-pyrido[3,4-b]indole;
(1S)-6-chloro-1-[6-(4-methylpiperazin-1-yl)pyridin-3-yl]-2-[4-(trifluoromethyl)pyrimidin-2-yl]-2,3,4,9-tetrahydro-1H-pyrido[3,4-b]indole;
(1S)-2-[4,6-bis(trifluoromethyl)pyrimidin-2-yl]-6-chloro-1-[6-(4-methylpiperazin-1-yl)pyridin-3-yl]-2,3,4,9-tetrahydro-1H-pyrido[3,4-b]indole;
2-[4,6-bis(trifluoromethyl)pyrimidin-2-yl]-1-[2-(morpholin-4-yl)pyrimidin-5-yl]-6-(2H-1,2,3-triazol-2-yl)-2,3,4,9-tetrahydro-1H-pyrido[3,4-b]indole;
2-[4,6-bis(trifluoromethyl)pyrimidin-2-yl]-6-(4-methyl-2H-1,2,3-triazol-2-yl)-1-[2-(morpholin-4-yl)pyrimidin-5-yl]-2,3,4,9-tetrahydro-1H-pyrido[3,4-b]indole;
(1S)-6-chloro-1-[2-(4-methylpiperazin-1-yl)pyrimidin-5-yl]-2-[4-(trifluoromethyl)pyrimidin-2-yl]-2,3,4,9-tetrahydro-1H-pyrido[3,4-b]indole;
(1S)-2-[4,6-bis(trifluoromethyl)pyrimidin-2-yl]-1-[6-(morpholin-4-yl)pyridin-3-yl]-6-(2H-1,2,3-triazol-2-yl)-2,3,4,9-tetrahydro-1H-pyrido[3,4-b]indole;
(1S)-2-[4,6-bis(trifluoromethyl)-1,3,5-triazin-2-yl]-1-[6-(morpholin-4-yl)pyridin-3-yl]-6-(2H-1,2,3-triazol-2-yl)-2,3,4,9-tetrahydro-1H-pyrido[3,4-b]indole;
(1S)-2-[4,6-bis(trifluoromethyl)pyrimidin-2-yl]-6-bromo-1-[6-(morpholin-4-yl)pyridin-3-yl]-2,3,4,9-tetrahydro-1H-pyrido[3,4-b]indole;
(1S)-6-chloro-1-[2-(4-methylpiperazin-1-yl)pyrimidin-5-yl]-2-[4-(trifluoromethyl)-1,3,5-triazin-2-yl]-2,3,4,9-tetrahydro-1H-pyrido[3,4-b]indole;
(1S)-6-chloro-1-[6-(4-methylpiperazin-1-yl)pyridin-3-yl]-2-[4-(methylsulfanyl)-6-(trifluoromethyl)-1,3,5-triazin-2-yl]-2,3,4,9-tetrahydro-1H-pyrido[3,4-b]indole;
2-[4,6-bis(trifluoromethyl)pyrimidin-2-yl]-1-(2,3-difluoro-4-methylphenyl)-6-fluoro-2,3,4,9-tetrahydro-1H-pyrido[3,4-b]indole;
(1S)-6-chloro-1-[6-(4-methylpiperazin-1-yl)pyridin-3-yl]-2-[4-(trifluoromethyl)-1,3,5-triazin-2-yl]-2,3,4,9-tetrahydro-1H-pyrido[3,4-b]indole;
2-[4,6-bis(trifluoromethyl)-1,3,5-triazin-2-yl]-1-(2,3-difluoro-4-methylphenyl)-6-fluoro-2,3,4,9-tetrahydro-1H-pyrido[3,4-b]indole;
1-(2,3-difluoro-4-methylphenyl)-6-fluoro-2-[4-(trifluoromethyl)pyrimidin-2-yl]-2,3,4,9-tetrahydro-1H-pyrido[3,4-b]indole;
1-(2,3-difluoro-4-methylphenyl)-6-fluoro-2-[4-(trifluoromethyl)-1,3,5-triazin-2-yl]-2,3,4,9-tetrahydro-1H-pyrido[3,4-b]indole;
1-(2,3-difluoro-4-methylphenyl)-6-fluoro-2-[4-(methylsulfanyl)-6-(trifluoromethyl)-1,3,5-triazin-2-yl]-2,3,4,9-tetrahydro-1H-pyrido[3,4-b]indole;
(1S)-2-[4,6-bis(trifluoromethyl)pyrimidin-2-yl]-6-chloro-1-[2-(3,3-dimethylpiperazin-1-yl)pyrimidin-5-yl]-2,3,4,9-tetrahydro-1H-pyrido[3,4-b]indole;
2-[4,6-bis(trifluoromethyl)-1,3,5-triazin-2-yl]-6-chloro-1-(2,3-difluoro-4-methylphenyl)-2,3,4,9-tetrahydro-1H-pyrido[3,4-b]indole;
(1S)-1-[6-(morpholin-4-yl)pyridin-3-yl]-6-(2H-1,2,3-triazol-2-yl)-2-[4-(trifluoromethyl)-1,3,5-triazin-2-yl]-2,3,4,9-tetrahydro-1H-pyrido[3,4-b]indole;
(1S)-2-[4,6-bis(trifluoromethyl)pyrimidin-2-yl]-6-bromo-1-[2-(4-methylpiperazin-1-yl)pyrimidin-5-yl]-2,3,4,9-tetrahydro-1H-pyrido[3,4-b]indole;
(1S)-2-[4,6-bis(trifluoromethyl)pyrimidin-2-yl]-1-[2-(4-methylpiperazin-1-yl)pyrimidin-5-yl]-6-(2H-1,2,3-triazol-2-yl)-2,3,4,9-tetrahydro-1H-pyrido[3,4-b]indole;
(1S)-2-[4,6-bis(trifluoromethyl)-1,3,5-triazin-2-yl]-1-[2-(4-methylpiperazin-1-yl)pyrimidin-5-yl]-6-(2H-1,2,3-triazol-2-yl)-2,3,4,9-tetrahydro-1H-pyrido[3,4-b]indole;
1-(2,3-difluoro-4-methylphenyl)-6-(2H-1,2,3-triazol-2-yl)-2-[4-(trifluoromethyl)pyrimidin-2-yl]-2,3,4,9-tetrahydro-1H-pyrido[3,4-b]indole;
(1S)-2-[4,6-bis(trifluoromethyl)pyrimidin-2-yl]-6-chloro-1-{2-[(3R,5S)-3,5-dimethylpiperazin-1-yl]pyrimidin-5-yl}-2,3,4,9-tetrahydro-1H-pyrido[3,4-b]indole;
methyl 4-[(1S)-6-chloro-1-(2,3-difluoro-4-methylphenyl)-1,3,4,9-tetrahydro-2H-pyrido[3,4-b]indol-2-yl]-6-(trifluoromethyl)-1,3,5-triazine-2-carboxylate;
(1S)-6-chloro-1-(2,3-difluoro-4-methylphenyl)-2-[4-(trichloromethyl)-6-(trifluoromethyl)-1,3,5-triazin-2-yl]-2,3,4,9-tetrahydro-1H-pyrido[3,4-b]indole;
(1S)-1-[2-(4-methylpiperazin-1-yl)pyrimidin-5-yl]-6-(2H-1,2,3-triazol-2-yl)-2-[4-(trifluoromethyl)-1,3,5-triazin-2-yl]-2,3,4,9-tetrahydro-1H-pyrido[3,4-b]indole;
(1S)-6-chloro-2-[4-(difluoromethyl)-6-(trifluoromethyl)-1,3,5-triazin-2-yl]-1-[2-(morpholin-4-yl)pyrimidin-5-yl]-2,3,4,9-tetrahydro-1H-pyrido[3,4-b]indole;
(1S)-6-chloro-2-[4-(difluoromethyl)-6-(trifluoromethyl)-1,3,5-triazin-2-yl]-1-[2-(4-methylpiperazin-1-yl)pyrimidin-5-yl]-2,3,4,9-tetrahydro-1H-pyrido[3,4-b]indole;
(1S)-6-chloro-1-[2-(4-methylpiperazin-1-yl)pyrimidin-5-yl]-2-[4-(morpholin-4-yl)-6-(trifluoromethyl)-1,3,5-triazin-2-yl]-2,3,4,9-tetrahydro-1H-pyrido[3,4-b]indole;
(1S)-6-chloro-1-(2,3-difluoro-4-methylphenyl)-2-[4-(difluoromethyl)-6-(trifluoromethyl)-1,3,5-triazin-2-yl]-2,3,4,9-tetrahydro-1H-pyrido[3,4-b]indole;
(1S)-2-[4,6-bis(trifluoromethyl)pyrimidin-2-yl]-6-chloro-1-{2-[(3R)-3-methylpiperazin-1-yl]pyrimidin-5-yl}-2,3,4,9-tetrahydro-1H-pyrido[3,4-b]indole;
(1S)-2-[4,6-bis(trifluoromethyl)pyrimidin-2-yl]-6-chloro-1-[2-(4,7-diazaspiro[2.5]octan-7-yl)pyrimidin-5-yl]-2,3,4,9-tetrahydro-1H-pyrido[3,4-b]indole;
1-[6-(4-methylpiperazin-1-yl)pyridin-3-yl]-6-(2H-1,2,3-triazol-2-yl)-2-[4-(trifluoromethyl)pyrimidin-2-yl]-2,3,4,9-tetrahydro-1H-pyrido[3,4-b]indole;
(1S)-2-[4,6-bis(trifluoromethyl)pyrimidin-2-yl]-1-[2-(morpholin-4-yl)pyrimidin-5-yl]-6-(2H-1,2,3-triazol-2-yl)-2,3,4,9-tetrahydro-1H-pyrido[3,4-b]indole;
(1S)-1-[2-(morpholin-4-yl)pyrimidin-5-yl]-6-(2H-1,2,3-triazol-2-yl)-2-[4-(trifluoromethyl)-1,3,5-triazin-2-yl]-2,3,4,9-tetrahydro-1H-pyrido[3,4-b]indole;
(1S)-2-[4,6-bis(trifluoromethyl)-1,3,5-triazin-2-yl]-1-[2-(morpholin-4-yl)pyrimidin-5-yl]-6-(2H-1,2,3-triazol-2-yl)-2,3,4,9-tetrahydro-1H-pyrido[3,4-b]indole;
(1S)-2-[4,6-bis(trifluoromethyl)pyrimidin-2-yl]-6-chloro-1-{2-[(3S)-3-methylpiperazin-1-yl]pyrimidin-5-yl}-2,3,4,9-tetrahydro-1H-pyrido[3,4-b]indole;
(1S)-2-[4,6-bis(trifluoromethyl)pyrimidin-2-yl]-6-bromo-1-(2,3-difluoro-4-methylphenyl)-2,3,4,9-tetrahydro-1H-pyrido[3,4-b]indole;
(1S)-2-[4,6-bis(trifluoromethyl)pyrimidin-2-yl]-1-(2,3-difluoro-4-methylphenyl)-6-(2H-1,2,3-triazol-2-yl)-2,3,4,9-tetrahydro-1H-pyrido[3,4-b]indole;
(1S)-2-[4,6-bis(trifluoromethyl)pyrimidin-2-yl]-1-(2,3-difluoro-4-methylphenyl)-6-(4-methyl-2H-1,2,3-triazol-2-yl)-2,3,4,9-tetrahydro-1H-pyrido[3,4-b]indole;
{4-[(1S)-6-chloro-1-(2,3-difluoro-4-methylphenyl)-1,3,4,9-tetrahydro-2H-pyrido[3,4-b]indol-2-yl]-6-(trifluoromethyl)-1,3,5-triazin-2-yl}methanol;
(1S)-2-[4,6-bis(trifluoromethyl)-1,3,5-triazin-2-yl]-6-chloro-1-(2-methoxypyrimidin-5-yl)-2,3,4,9-tetrahydro-1H-pyrido[3,4-b]indole;
(1S)-2-[4,6-bis(trifluoromethyl)-1,3,5-triazin-2-yl]-6-bromo-1-(2,3-difluoro-4-methylphenyl)-2,3,4,9-tetrahydro-1H-pyrido[3,4-b]indole;
(1S)-2-[4,6-bis(trifluoromethyl)-1,3,5-triazin-2-yl]-1-(2,3-difluoro-4-methylphenyl)-6-(2H-1,2,3-triazol-2-yl)-2,3,4,9-tetrahydro-1H-pyrido[3,4-b]indole;
(1S)-2-[4,6-bis(trifluoromethyl)-1,3,5-triazin-2-yl]-1-(2,3-difluoro-4-methylphenyl)-6-(4-methyl-2H-1,2,3-triazol-2-yl)-2,3,4,9-tetrahydro-1H-pyrido[3,4-b]indole;
(1S)-6-chloro-1-(2,3-difluoro-4-methylphenyl)-2-[4-(fluoromethyl)-6-(trifluoromethyl)-1,3,5-triazin-2-yl]-2,3,4,9-tetrahydro-1H-pyrido[3,4-b]indole;
(1S)-2-[4,6-bis(trifluoromethyl)-1,3,5-triazin-2-yl]-1-(2,3-difluoro-4-methylphenyl)-6-nitro-2,3,4,9-tetrahydro-1H-pyrido[3,4-b]indole;
(1S)-2-[4,6-bis(trifluoromethyl)pyrimidin-2-yl]-1-(2,3-difluoro-4-methylphenyl)-6-methoxy-2,3,4,9-tetrahydro-1H-pyrido[3,4-b]indole;
(1S)-2-[4,6-bis(trifluoromethyl)-1,3,5-triazin-2-yl]-1-(2,3-difluoro-4-methylphenyl)-6-(4-methyl-1H-pyrazol-1-yl)-2,3,4,9-tetrahydro-1H-pyrido[3,4-b]indole;
(1S)-2-[4,6-bis(trifluoromethyl)-1,3,5-triazin-2-yl]-1-(2,3-difluoro-4-methylphenyl)-6-(3-methyl-1H-pyrazol-1-yl)-2,3,4,9-tetrahydro-1H-pyrido[3,4-b]indole;
N²-(5-{(1S)-2-[4,6-bis(trifluoromethyl)-1,3,5-triazin-2-yl]-6-chloro-2,3,4,9-tetrahydro-1H-pyrido[3,4-b]indol-1-yl}pyrimidin-2-yl)-N¹,N¹-dimethylethane-1,2-diamine;
methyl 5-{(1S)-2-[4,6-bis(trifluoromethyl)-1,3,5-triazin-2-yl]-6-chloro-2,3,4,9-tetrahydro-1H-pyrido[3,4-b]indol-1-yl}pyrimidine-2-carboxylate;
1-(5-{(1S)-2-[4,6-bis(trifluoromethyl)-1,3,5-triazin-2-yl]-6-chloro-2,3,4,9-tetrahydro-1H-pyrido[3,4-b]indol-1-yl}pyrimidin-2-yl)ethan-1-one;
(1S)-2-[4,6-bis(trifluoromethyl)-1,3,5-triazin-2-yl]-6-chloro-1-[2-(2-methoxyethoxy)pyrimidin-5-yl]-2,3,4,9-tetrahydro-1H-pyrido[3,4-b]indole;
2-[(5-((1S)-2-[4,6-bis(trifluoromethyl)-1,3,5-triazin-2-yl]-6-chloro-2,3,4,9-tetrahydro-1H-pyrido[3,4-b]indol-1-yl)pyrimidin-2-yl)amino]ethan-1-ol;
2-[4,6-bis(trifluoromethyl)-1,3,5-triazin-2-yl]-6-chloro-1-[2-(2-methoxyethoxy)pyrimidin-5-yl]-2,3,4,9-tetrahydro-1H-pyrido[3,4-b]indole;
N-(5-{(1S)-2-[4,6-bis(trifluoromethyl)-1,3,5-triazin-2-yl]-6-chloro-2,3,4,9-tetrahydro-1H-pyrido[3,4-b]indol-1-yl}pyrimidin-2-yl)-p-alanine;
4-[(5-{(1S)-2-[4,6-bis(trifluoromethyl)-1,3,5-triazin-2-yl}]-6-chloro-2,3,4,9-tetrahydro-1H-pyrido[3,4-b]indol-1-yl}pyrimidin-2-yl)amino]butanoic acid; and
{2-[(5-{(1S)-2-[4,6-bis(trifluoromethyl)-1,3,5-triazin-2-yl]-6-chloro-2,3,4,9-tetrahydro-1H-pyrido[3,4-b]indol-1-yl}pyrimidin-2-yl)amino]ethoxy}acetic acid;
or a form thereof selected from the group consisting of a salt, hydrate, solvate, racemate, enantiomer, diastereomer, stereoisomer, and tautomer.

11. A compound salt selected from the group consisting of:
2-[4,6-bis(trifluoromethyl)pyrimidin-2-yl]-6-chloro-1-[6-(morpholin-4-yl)pyridin-3-yl]-2,3,4,9-tetrahydro-1H-pyrido[3,4-b]indole formate (1:1);
2-[4,6-bis(trifluoromethyl)pyrimidin-2-yl]-6-chloro-1-[2-(morpholin-4-yl)pyrimidin-5-yl]-2,3,4,9-tetrahydro-1H-pyrido[3,4-b]indole formate (1:1); and,
(1S)-6-chloro-1-[6-(4-methylpiperazin-1-yl)pyridin-3-yl]-2-[4-(trifluoromethyl)-1,3,5-triazin-2-yl]-2,3,4,9-tetrahydro-1H-pyrido[3,4-b]indole formate (1:1);
or a form thereof selected from the group consisting of a hydrate, solvate, racemate, enantiomer, diastereomer, stereoisomer, and tautomer.

12. A method of use of a compound of Formula (I), or a form thereof, of claim 1 to treat or ameliorate a disease or disorder in a subject in need thereof comprising, administering to the subject an effective amount of a compound of Formula (I), or a form thereof, to inhibit dihydroorotate dehydrogenase.

13. A pharmaceutical composition comprising a compound of Formula (I), or a form thereof, of claim 1 and a pharmaceutically acceptable excipient to treat or ameliorate a disease or disorder by inhibiting dihydroorotate dehydrogenase.

14. A method of use of a compound of Formula (I), or a form thereof, of claim 10 to treat or ameliorate a disease or disorder in a subject in need thereof comprising, administering to the subject an effective amount of a compound of Formula (I), or a form thereof, to inhibit dihydroorotate dehydrogenase.

15. A method of use of a compound of Formula (I), or a form thereof, of claim 11 to treat or ameliorate a disease or disorder in a subject in need thereof comprising, administering to the subject an effective amount of a compound of Formula (I), or a form thereof, to inhibit dihydroorotate dehydrogenase.

16. A pharmaceutical composition comprising a compound of Formula (I), or a form thereof, of claim 10 and a pharmaceutically acceptable excipient to treat or ameliorate a disease or disorder by inhibiting dihydroorotate dehydrogenase.

17. A pharmaceutical composition comprising a compound of Formula (I), or a form thereof, of claim 11 and a pharmaceutically acceptable excipient to treat or ameliorate a disease or disorder by inhibiting dihydroorotate dehydrogenase.
